SEQUENCE LISTING (12 sequences)

SEQ ID NO: 1 - protein, 60 aa, Pseudomonas aeruginosea
  NON_TER 1,60
RNGGQILVEALRRNAVDTVYCIPGESYLPVLDALYDTDGIRTVVTRHEGAASNMADAYGK

SEQ ID NO: 2 - protein, 21 aa, Artificial Sequence
  Description of Artifical Sequence Sequence comparison of SEQ ID NO 1 to SEQ ID NO 3
RGGLRVGPLAGIRVTRHEADA

SEQ ID NO: 3 - protein, 59 aa, Homo sapiens
  NON_TER 1,59
  NON_CONS (34)...(35)
RHGGENVAAVLRAHGVRFIFTLVGGHISPLLVACEKLGIRVVDTRHEVTGVFAADAMAR

SEQ ID NO: 4 - protein, 60 aa, Pseudomonas aeruginosea
  NON_TER 1,60
LTGRPGICFVTRGPGATHAANGVHTAQQDSTPMILFVGQVESAFKGREAFQEVDYVQMFS

SEQ ID NO: 5 - protein, 21 aa, Artificial Sequence
  Description of Artifical Sequence Sequence comparison of SEQ ID NO 4 to SEQ ID NO 6
LGGVTGPGTVAQPLGRAQVDF

SEQ ID NO: 6 - protein, 60 aa, Homo sapiens
  NON_TER 1,60
LSGTVGVAAVTAGPGLTNTVTAVKNAQMAQSPILLLGGAASTLLQNRGALQAVDQLSLFR

SEQ ID NO: 7 - protein, 55 aa, Pseudomonas aeruginosea
  NON_TER 1,55
GLAKWAVEIDRIERIPEIVGRAFSVATSGRPGPVVVALPEEILFGSAQVADAPEP

SEQ ID NO: 8 - protein, 18 aa, Artificial Sequence
  Description of Artifical Sequence Sequence comparison of SEQ ID NO 7 to SEQ ID NO 9
LKVRIASGPGPVVLPLVP

SEQ ID NO: 9 - protein, 55 aa, Homo sapiens
  NON_TER 1,55
  MOD_RES (22): Any Amino Acid
PLCKFCVSVPRVRDIVPTLRAXMAAAQSGTPGPVFVELPVDVLYPFFMVQKEMVP

SEQ ID NO: 10 - protein, 54 aa, Pseudomonas aeruginosea
  NON_TER 1,54
LLLENEPGALSRVVGLFSQRNYNIESLTVAPTEDPTLSRLTLTTVGHDEVIEQI

SEQ ID NO: 11 - protein, 18 aa, Artificial Sequence
  Description of Artifical Sequence Sequence comparison of SEQ ID NO 10 to SEQ ID NO 12
LLLPGLRNNADPLGHEVI

SEQ ID NO: 12 - protein, 53 aa, Homo sapiens
  NON_TER 1,53
  NON_CONS (42)..(43)
LLLLSLPGLAAGITILLTDRNLNTTFFDPAGGGDPILYQHLFIFGHPEVYNRI

(19) United States
(12) Patent Application Publication
(10) Pub. No.: US 2003/0130179 A1
(43) Pub. Date: Jul. 10, 2003

(54) Methods for identifying therapeutic targets for treating infectious disease

(76) Inventors: H. Michael Shepard, Encinitas, CA (US); David B. Lackey, San Diego, CA (US); Brian E. Cathers, San Diego, CA (US); Maria V. Sergeeva, San Diego, CA (US)

Correspondence Address: Antoinette F. Konski, McCutchen, Doyle, Brown & Enersen, LLP, 18th Floor, Three Embarcadero Center, San Francisco, CA, 94111, US

(21) Appl. No.: 09/910,345

(22) Filed: Jul. 20, 2001

Related U.S. Application Data
(60) Provisional application No. 60/219,598, filed on Jul. 20, 2000.
(60) Provisional application No. 60/244,953, filed on Nov. 1, 2000.
(60) Provisional application No. 60/276,728, filed on Mar. 16, 2001.

(51) Int. Cl. A61K 38/17; C12Q 1/70; G01N 33/53

(52) U.S. Cl. 514/12; 435/5; 435/7.1

(57) ABSTRACT

This invention provides methods and systems to identify enzymes that act as enzyme catalyzed therapeutic activators and the enzymes identified by these methods. Also provided by this invention are compounds activated by the enzymes as well as compositions containing these compounds.

### CROSS-REFERENCE TO RELATED APPLICATIONS

This application claims the benefit under 35 U.S.C. §119(e) of U.S. provisional patent applications having the serial Nos. 60/219,598; 60/244,953; and 60/276,728, filed Jul. 20, 2000; Nov. 1, 2000; and Mar. 16, 2001, respectively. The contents of these applications are hereby incorporated by reference into the present disclosure.

### TECHNICAL FIELD

The present invention relates to the field of Enzyme Catalyzed Therapeutic Activation (ECTA™) therapy and in particular, ECTA therapies targeting intrinsic and unique enzymes in pathogenic microorganisms or in host cells.

### BACKGROUND OF THE INVENTION

Throughout and within this disclosure, various publications, patents, published patent applications and references are identified by first author and date, within parentheses, patent number, publication number or by web address. If the complete bibliographic citation is not provided after the publication or reference, it is at the end of the specification, immediately preceding the claims. The disclosures of all publications, references and information provided at the web addresses are hereby incorporated by reference into this disclosure to more fully describe the state of the art to which this invention pertains.

The resistance of bacterial, fungal, and viral pathogens to drug therapy has become a health issue of global concern. Similarly, the resistance of cancer cells to chemotherapy is responsible for about 600,000 deaths each year in the United States. While there are important differences that distinguish these different diseases, there are also important unifying concepts. For this reason, the introduction to this patent application will focus on bacterial drug resistance mechanisms, and refer to common issues with other diseases as appropriate.

When antibiotics became widely available in the middle of the twentieth century (approximately 50 years ago), they were hailed as miracle drugs—magic bullets able to eliminate bacterial infection without harm to the normal cells of treated individuals. Yet with each passing decade, drug-resistant bacteria and other drug-resistant pathogens have emerged with increasing frequency. Simple bacterial and fungal infections that were once eliminated with a single drug and a simple course of therapy have become life threatening, and can be successfully treated only with drugs that display significant toxicity. Similarly, the tremendous optimism that followed initial clinical use of protease and reverse transcriptase inhibitors in the treatment of human immunodeficiency disease has now been replaced with complex cocktails of agents, and the understanding that resistant strains of virus will develop (Armstrong and Cohen (1999)).

An important reason why resistance has continued to develop at such a rapid rate, in all fields of infectious disease, is that the discovery and development of antibiotics has focused on only a few targets and a few mechanisms. By far the most common approach to discovery of anti-infectives has been the search for inhibitors of bacterial, fungal or viral enzyme functions. Antibiotics to treat the most common bacterial infections attack only a few distinct targets in the pathogen (Neu (1992)). For example, beta-lactams, penicillins, cephalosporins, monobactams, carbapenems, and penems are Class I inhibitors of bacterial cell wall synthesis. The glycopeptides vancomycin and teicoplanin are examples of Class II inhibitors of cell wall synthesis. Clindamycin, chloramphenicol, tetracyclines, and aminoglycosides are examples of known inhibitors of protein synthesis. Ciprofloxacin and ofloxacin are known inhibitors of DNA gyrase.

Each of these drugs targets an important enzyme. For difficult infections, combinations of these and other drugs are often utilized. However, the combination of drugs often works toward inhibition of separate enzyme targets. For instance, in bacterial and sometimes fungal infections, the combination of trimethoprim and sulfomethoxazole is used to simultaneously inhibit dihydrofolate reductase and dihydroopterate synthetase, respectively. Similar approaches are used for the treatment of viral infections and cancer. In anti-HIV therapy the combination of reverse transcriptase and viral protease inhibitors is commonly employed. In treatment of breast cancer, cocktails that include a fluoropyrimidine and methotrexate, inhibitors of thymidylate synthase and dihydrofolate reductase, respectively, are often used.

It can therefore be seen that inhibitors of enzyme function are favored for the development of drug treatments in cancer and infectious disease. However, this approach has led to the emergence of drug resistant strains that render the original therapeutic ineffective. Antimicrobial agents are rendered inactive by four major mechanisms (Reviewed in Schmitz and Fluit (1999)):

(I). Enzyme mediated. This is the most common inactivation scheme observed in laboratory and clinical bacterial strains. For example, beta-lactam antibiotics work by inhibiting cell wall synthesis, specifically they inhibit penicillin binding protein. In some cases though, bacteria express a beta-lactamase enzyme which hydrolyses the antibiotics so they become inactive. Bacteria that express a beta-lactamase are often beta-lactam-antibiotic resistant. Pathogens can also enzymatically modify a therapeutic so that it cannot bind to its target (as seen with aminoglycosides and chloramphenicol). In both of the cases outlined above, an enzyme encoded by the pathogen mediates resistance.

(II). Membrane permeability. Pathogens adapt and change their cell wall (the porin structures) to prevent drug entry. This can occur in response to almost any antibacterial agent.

(III). Drug efflux pumps. Pathogens adapt and change membrane transport proteins (also an enzyme family), such that they operate with increased efficiency toward the antibiotic. This is an important mechanism of resistance to tetracycline.

(IV). Target mutation. Pathogens mutate the therapeutic target thereby preventing activation of the antibiotic. Common mutations occur in the penicillin binding protein, which prevents activation of the antibiotic.

In the hospital setting, the most recent worrisome resistance traits to emerge include plasmid-mediated resistance to imipenem and to third-generation cephalosporins among nosocomial gram-negative bacteria, and the acquisition of resistance to vancomycin by enterococci. Methicillin-resistant staphylococci continue to be a problem, with about 75% of clinical strains found to be resistant to the penicillin-related drugs, and increasingly resistant to numerous other agents. The most important resistance traits seen in community-acquired organisms include beta-lactam resistance in Streptococcus pneumoniae and combined ampicillin and chloramphenicol resistance in Haemophilus influenzae. Shigellae resistant to essentially all commonly used oral agents are also a problem, particularly in developing countries (Reviewed by Murray, B. (1997)).

While there are important differences in the exact mechanisms of drug resistance between bacterial, fungal and viral pathogens, a common theme is present throughout. Most commonly, enzyme inhibitors are selected for drug development and use in the clinic. Similarly, inhibitors of enzyme function are commonly used in the treatment of cancer (McVie (1999)). In each of these cases, drug resistance is characterized by increased enzyme expression, mutation of the target enzyme (so that it no longer recognizes the inhibitor), changes in target cell permeability and the development or overexpression of efflux pumps. There is no end is in sight to the problem of drug resistance and, thus, new strategies to prevent and control resistant pathogens and tumor cells continue to be necessary.

Thus, a need exists for a novel approach to the development of anti-infective agents that overcome the drawbacks of current inhibitor-based therapeutic approaches. Various aspects of this invention satisfy this need by providing methods and systems to identify target enzymes and methods to design and assay novel therapeutic prodrugs activated by these enzymes.

### DISCLOSURE OF THE INVENTION

This invention provides a vertically integrated drug discovery program that Applicant has utilized to identify therapeutic enzyme targets and which can be used to identify prodrugs which act by a unique mechanism of action (termed “Enzyme Catalyzed Therapuetic Activation” or “ECTA”™). In one aspect, the invention provides systems and methods to identify enzyme targets in silico. In alternative aspect, the invention provides a method to design potential prodrugs activated by the enzyme targets. In a yet further aspect of this invention, in vitro and in vivo assays are provided. The assays and prodrugs also are useful to test potential therapeutics. Further provided are methods to inhibit the growth of target organisms, cells, or host cells using the prodrugs of this invention. Methods to treat or alleviate the symptoms of selected diseases are further provided using the prodrugs of this invention.

In one aspect, the in silico methods comprise selecting from a suitable database an enzyme or list of enzymes expressed by a target organism, by an infectious agent or in an infected host cell, or by or in a pathological cell. The results of this search are compared against a search of expressed enzymes in or by a suitable control The method selects for enzymes expressed in one cell type or organism but not in another. Various embodiments of this aspect are provided herein. For example, one embodiment identifies enzymes expressed by a pathogen or on in a pathogen-infected cell but not expressed in the host or uninfected host cell.

These methods identified and will identify enzymes that are targets (“target enzymes”) for a novel ECTA approach to treat a variety of diseases including bacterial, fungal, parasitic and viral infections. In contrast, conventional therapies rely on the use of inhibitors of enzymes critical for target viability and/or proliferation. Consistent with Applicant's ECTA approach, the prodrug compounds of this invention do not act as enzyme inhibitors but undergo enzyme catalyzed transformation by target enzymes resulting in the generation of cytotoxic reaction product(s). The formation of cytotoxic species is achieved by engineering unique substrates (ECTA prodrugs or compounds) which are transformed into toxins by the target enzymes.

In one aspect, the target enzymes of this invention are pathogen-specific enzymes that are only expressed by pathogens, e.g., bacterial and fungal pathogens or in virally infected cells. In cases of intracellular parasites or viruses, host cell enzymes induced by the pathogen or infectious agent, or enzymes specifically encoded by the pathogen or infectious agent, were targeted and can be targeted in further embodiments of this invention.

The pharmaceutical and agricultural industries have focused on development of inhibitors of selected target enzymes for the development of anti-infectives, insecticides and herbicides (Shaner and Singh (1997) and Papamichael (1999)). This approach has suffered from several issues: (1) the presence of salvage pathways which allow specific enzyme inhibition to be circumvented; (2) mutation of the enzyme so that it no longer binds inhibitor, but can still metabolize substrate; and (3) inhibitor-associated enzyme overexpression leading to resistance.

The use of enzyme inhibitors for treatment can often result in harmful and uncomfortable side effects. For example, protease inhibitors used in HIV treatment have been shown to affect glucose control, lipid metabolism, and body fat distribution (Mulligan (2000)).

This invention defines a new ECTA approach that targets intrinsic enzymes (“iECTA” approach) which overcomes the limitations and problems associated with prior art therapies. Applicant's approach is distinguished from prior approaches because iECTA enzymes are NOT endogenous enzymes for the host cell and are not necessarily related to drug resistance. In other words, only pathogens or pathogen-infected cells express the iECTA enzymes. The prodrug compounds which are designed to be selectively activated by the iECTA enzymes also avoid side effects by achieving alternative, more selective therapies that preferentially affect diseased cells with little or no effect on healthy tissue. To the best of Applicant's knowledge, this approach has not been described or utilized previously. Therapeutics designed and generated using iECTA technology supplement and complement present day enzyme inhibitor-based treatments.

The present method can be applied to identify target enzymes other than iECTA enzymes by searching a first suitable data structure (database) to obtain a first set of information relating to one or more enzymes associated with a target organism. In certain embodiments, the enzyme is overexpressed or selectively expressed as compared to a control counterpart. A search also is conducted on one or more other suitable data structures (databases) to obtain one or more additional sets of information relating to one or more expressed enzymes associated with one or more additional class of organisms or by the same organism growing under in a different environment or in a different host. The first set of information is compared to the one or more additional sets of information to identify enzymes in the first set of information that are not present in the one or more additional sets of information. These identified enzymes are targets for ECTA compounds.

This invention further provides ECTA prodrugs. While each prodrug is selectively activated by a specific target enzyme counterpart, there are some general features of ECTA prodrugs. FIGS. 2A and 2B describe general characteristics of ECTA prodrugs that distinguish them from conventional therapeutics. One feature of an ECTA enzyme/ECTA compound combination is the absence of irreversible inhibition or inactivation of the target enzyme by the ECTA compound, intermediates or products of the reaction. In some embodiments, it is preferred but not necessary, that the ECTA target enzyme be critical for disease progression. This property means that resistance to iECTA compounds, which could result from disease/loss of target enzyme expression, may also result in decreased pathogenicity. To the best of Applicant's knowledge, this approach has not been previously described or utilized.

Also provided is a method for the design of iECTA compounds or prodrugs which are selectively activated by yet to be identified iECTA enzymes using the methods of this invention. This invention further provides iECTA compounds or prodrugs activated by infectious agents or in host cells, e.g., the enzymes listed in FIG. 7A and 7B and their biological equivalents. As used herein, and unless specifically excluded, Applicants intend for the biological equivalents of the iECTA compounds to be included in each embodiment of the invention. A “biological equivalent” is defined infra.

The iECTA compounds are provided alone or in combination with a liquid or solid carrier. Compositions comprising at least one iECTA compound or its biological equivalent in combination with an additional therapeutic is further provided by this invention.

Also provided is an assay for an iECTA compound that selectively inhibits the growth of an infectious agent in a target cell or an infected cell. The iECTA prodrug is contacted with its target enzyme in a cell-free system under suitable conditions. Activation by the target enzyme is monitored by methods well known in the art.

An in vitro screen is further provided by this invention. The iECTA enzyme is contacted with a pathogen or host cell containing or expressing the target enzyme. In one embodiment, the host cell and the prodrug are contacted under conditions that that favor incorporation of the compound into the host cell. The pathogens or host cells are assayed for inhibition of growth or killing of the infectious agent or the host cell. Control systems and/or cells can be contacted with the prodrug and assayed.

This invention also provides a method for inhibiting the growth or proliferation of an infectious agent or a host cell by contacting the infectious agent or host cell with an effective amount of an ECTA prodrug, e.g., an iECTA prodrug.

A method for determining whether a subject will be suitably treated by an ECTA prodrug such as an iECTA prodrug is provided by this invention. As an example, an iECTA compound is delivered to an infected cell under suitable conditions such that the growth of the infectious agent or infected cell is inhibited or the agent is killed.

Various modifications of the above methods are within the scope of this invention. For example, a different and/or additional enzyme target can be assayed against the same iECTA prodrug or a different and/or additional prodrug can be assayed against the same target enzyme. Prior art therapeutics or therapeutic methods can be combined with the use of the iECTA prodrugs to enhance or modify the biological activity of the iECTA prodrug. These methods can also be modified by varying the amount of the iECTA prodrug and/or additional therapeutic or alternatively or in combination, the order of the prodrugs and/or therapies can be modified, e.g., simultaneous or sequential. The sequential order can further be modified. These methods are further modified for prophylactic use.

A kit for determining whether a pathogen or pathogen-infected cell will be suitably treated by an iECTA therapy is also provided by this invention. The kit comprises an effective amount of at least one compound of this invention and instructions for use.

As is apparent to those of skill in the art, the above iECTA methods can be modified for application in other ECTA systems. These systems are described in more detail below.

### BRIEF DESCRIPTION OF THE FIGURES

FIG. 1 shows how ECTA technology preferentially targets selected cells.

FIGS. 2A and 2B are the process for successful identification of one embodiment of this invention, the identification of iECTA target enzymes and iECTA compounds.

FIG. 2C is a flowchart for a process for identifying enzymes for designing ECTA compounds in accordance with an embodiment of the present invention.

FIG. 2D is a schematic diagram of an illustrative system capable of executing the process for identifying enzymes for designing ECTA compounds set forth in FIG. 2C in accordance with an embodiment of the present invention.

FIG. 3 shows the results of one embodiment of the method of this invention that utilizes tBLAST alignment for the identification of a “favorable reaction type” iECTA. Shown in the figure is a tBLAST alignment of Pseudomonas aeruginosea acetolactate synthase large subunit amino acid sequence with the human expressed sequence tag database (translated in all six possible reading frames). The low “Expect” (E) values indicate that it is extremely unlikely that any of these alignments could occur by chance alone. Only the ten best E values and the best alignment are shown.

FIG. 4 shows the results of one embodiment of the method of this invention that utilizes tBLAST alignment for the identification of a “favorable reaction type” iECTA. Shown in the figure is a tBLAST alignment of Pseudomonas aeruginosea acetolactate synthase small subunit amino acid sequence with the human expressed sequence tag database (translated in all six possible reading frames). E values=6.5 indicating that the alignment shown would be predicted to be found more than six times in the expresses tag database due to chance alone.

FIG. 5 is a proposed mechanism of AcLS ECTA.

FIG. 6 is a comparison of 2-oxobutyrate metabolism in humans and E. coli.

FIG. 7A is a list of the Enzyme Commission Numbers representing intrinsic and unique enzymes for the following organisms:

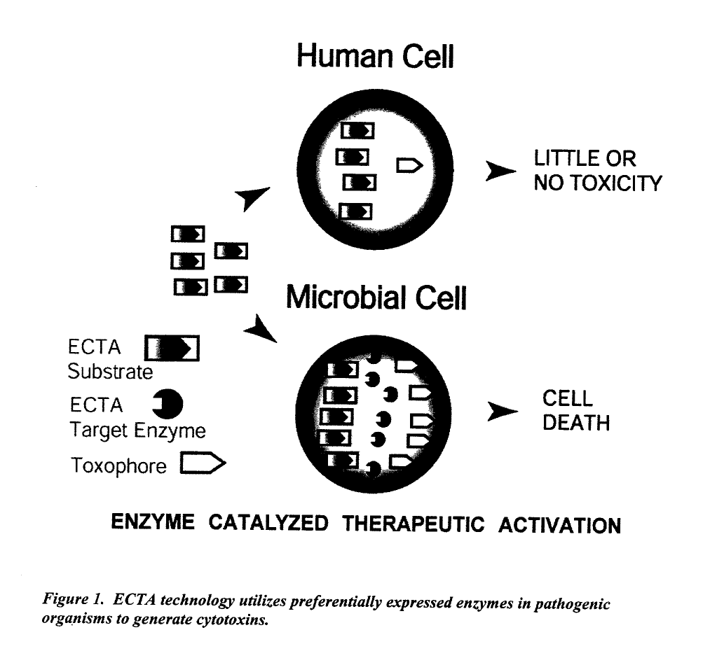

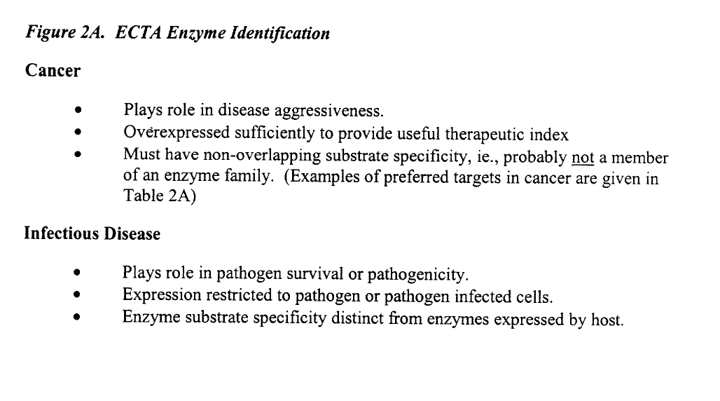

1. “Yersinia pseudotuberculosis”

2. “Yersinia pestis”

3. “Vibrio cholerae El Tor N16961”

4. “Ureaplasma urealyticum”

5. “Treponema pallidum”

6. “Streptomyces coelicolor”

7. “Streptomyces coelicolor”

8. “Streptococcus pyogenes”

9. “Streptococcus pneumonia”

10. “Streptococcus mutans”

11. “Streptococcus equi”

12. “Staphylococcus aureus”

13. “Salmonella typhimurium”

14. “Salmonella typhi”

15. “Salmonella paratyphi”

16. “Salmonella enteritidis”

17. “Salmonella dublin”

18. “Saccharomyces cerevisia”

19. “Rickettsia prowazekii”

20. “Pseudomonas aeruginosa”

21. “Porphyromonas gingivalis”

22. “Pasteurella multocida”

23. “Neurospora crassa”

24. “Neisseria meningitidis ser. B”

25. “Neisseria meningitidis ser. A”

26. “Neisseria gonorrhoeae”

27. “Mycoplasma pneumoniae”

28. “Mycoplasma genitalium”

29. “Mycobacterium tuberculosis”

30. “Mycobacterium leprae”

31. “Mycobacterium bovis”

32. “Klebsiella pneumoniae”

33. “Helicobacter pylori”

34. “Helicobacter pylori J99”

35. “Haemophilus influenzae”

36. “Haemophilus ducreyi”

37. “Escherichia coli”

38. “Enterococcus faecium (DOE)”

39. “Enterococcus faecalis”

40. “Corynebacterium diphthe”

41. “Clostridium difficile”

42. “Clostridium acetobutyli”

43. “Chlamydia trachomatis D”

44. “Chlamydia trachomatis M”

45. “Chlamydia pneumoniae AR39”

46. “Chlamydia pneumoniae CWL029”

47. “Campylobacter jejuni”

48. “Borrelia burgdorferi”

49. “Bordetella pertussis”

50. “Bordetella bronchiseptica”

51. “Bacillus subtilis”

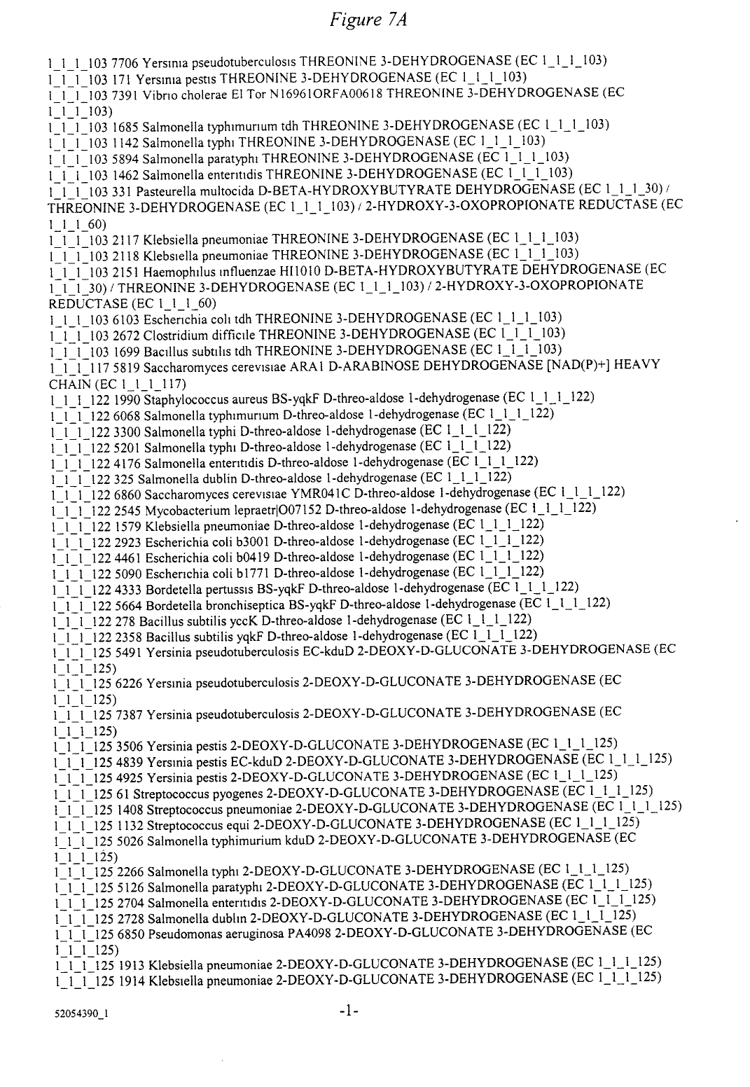

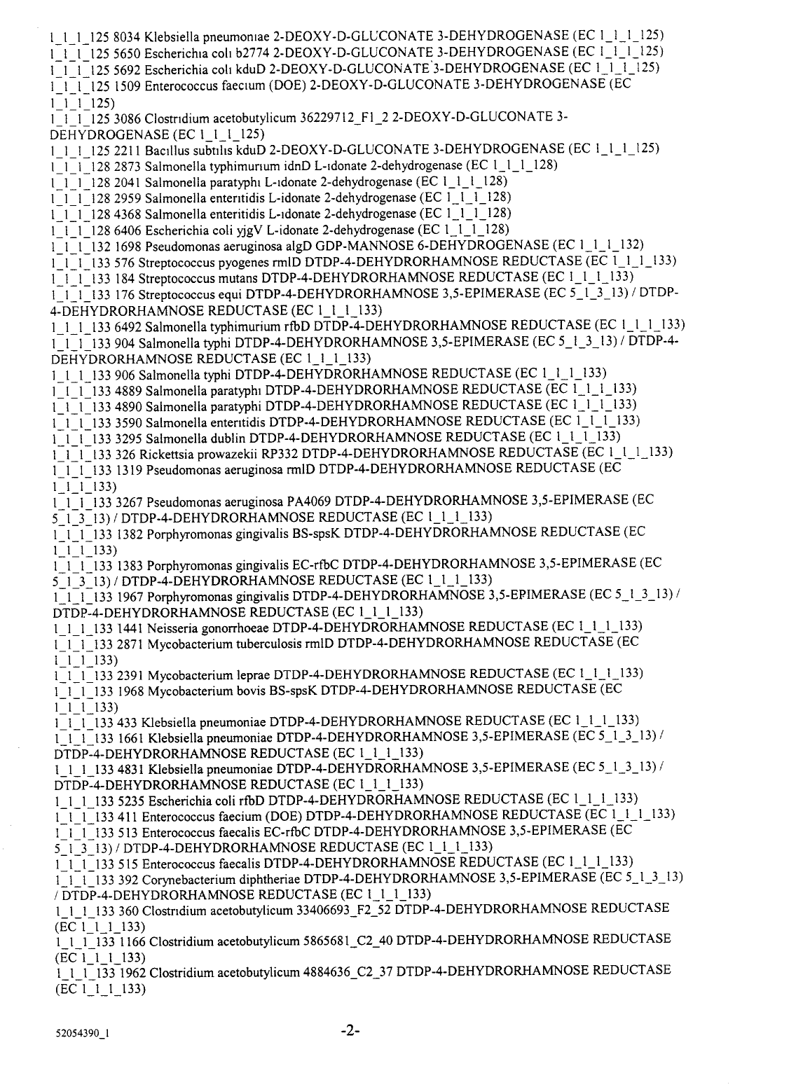

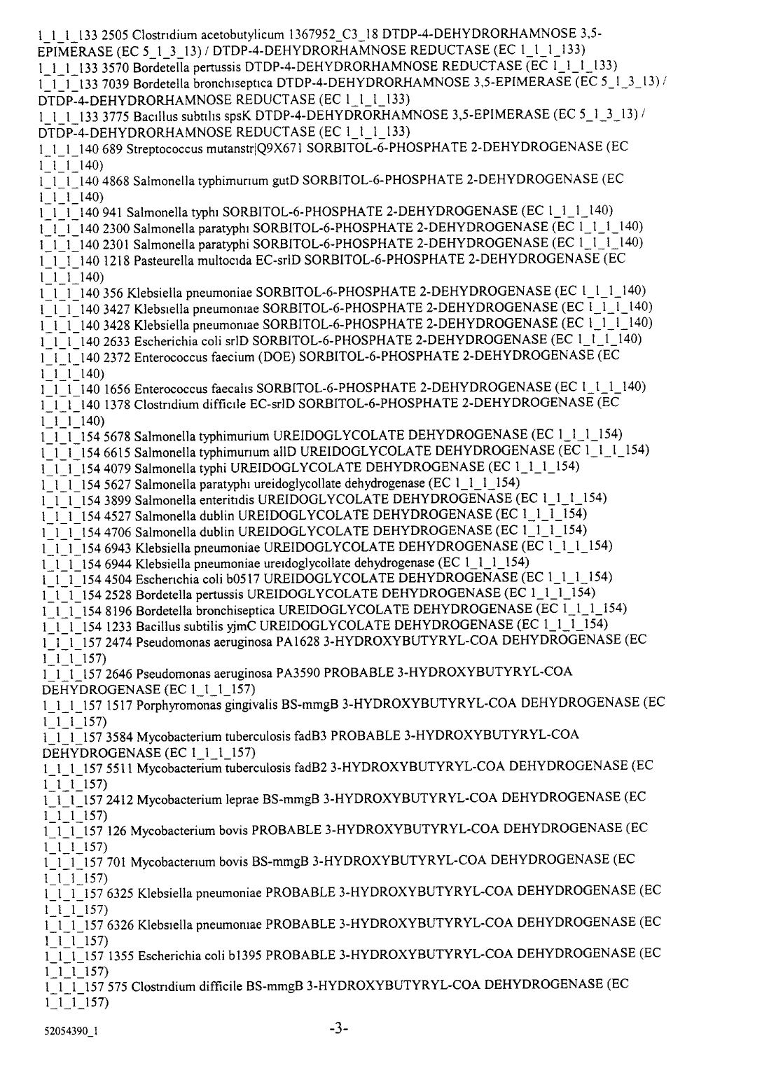

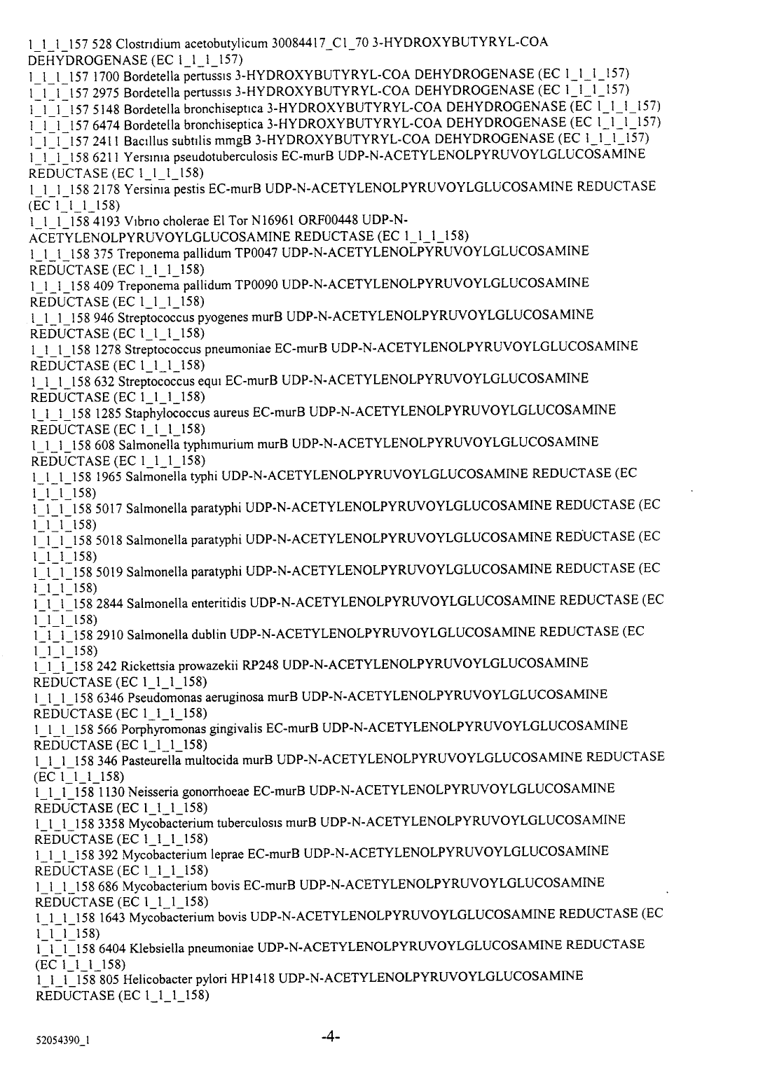

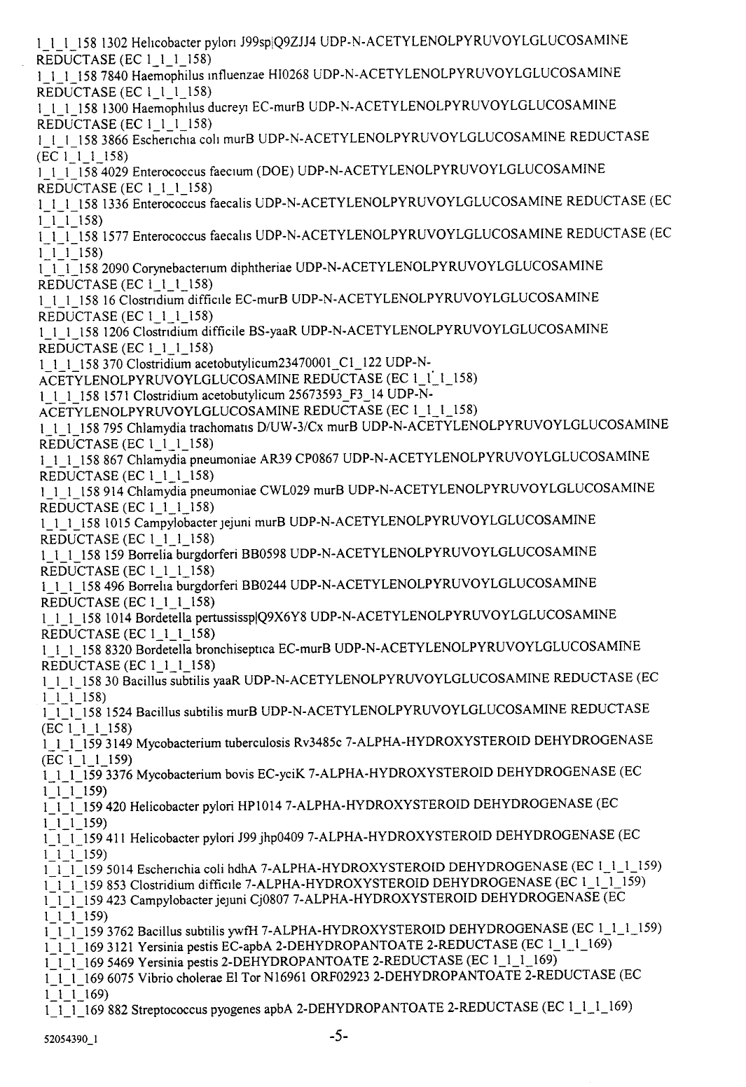

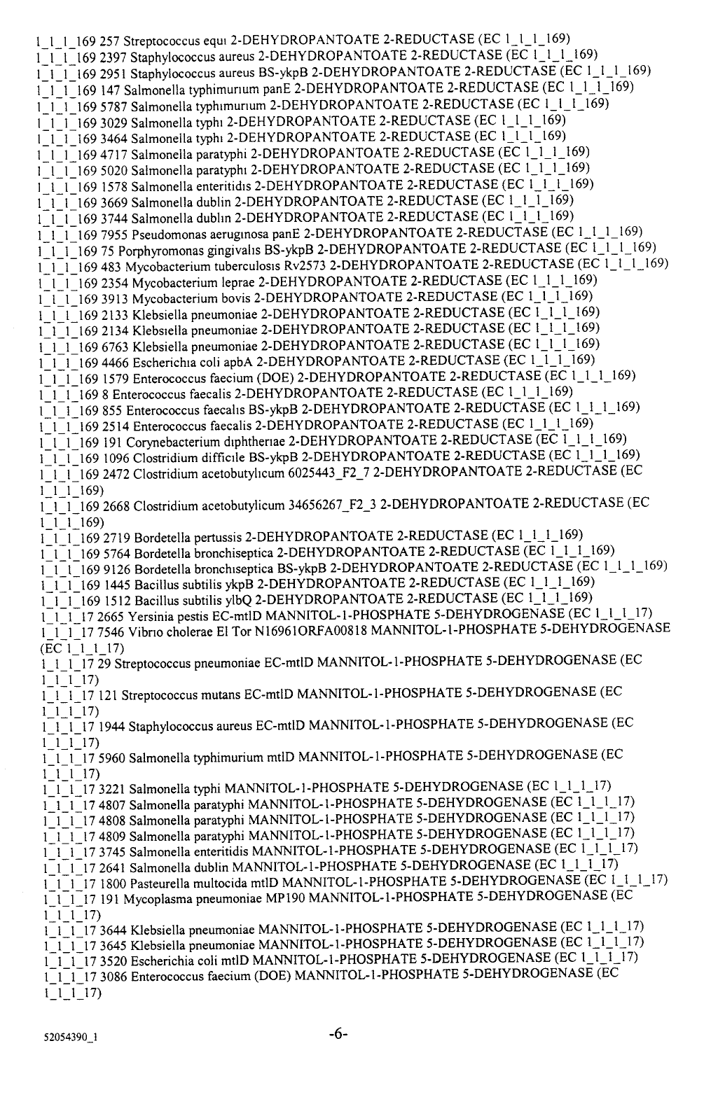

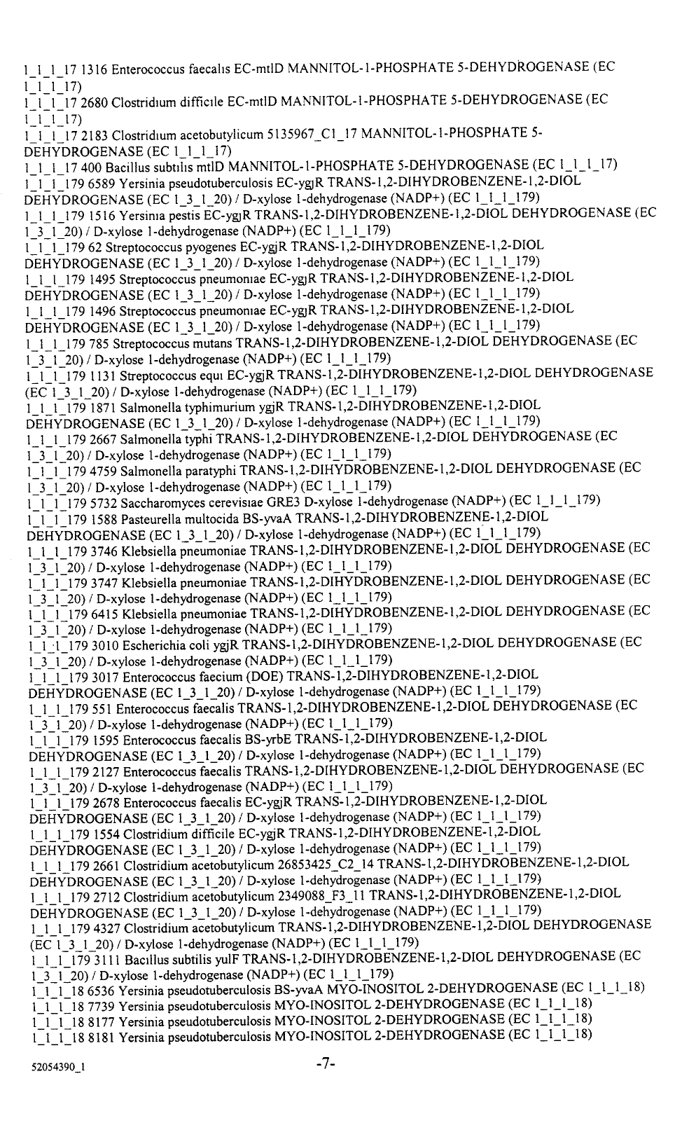

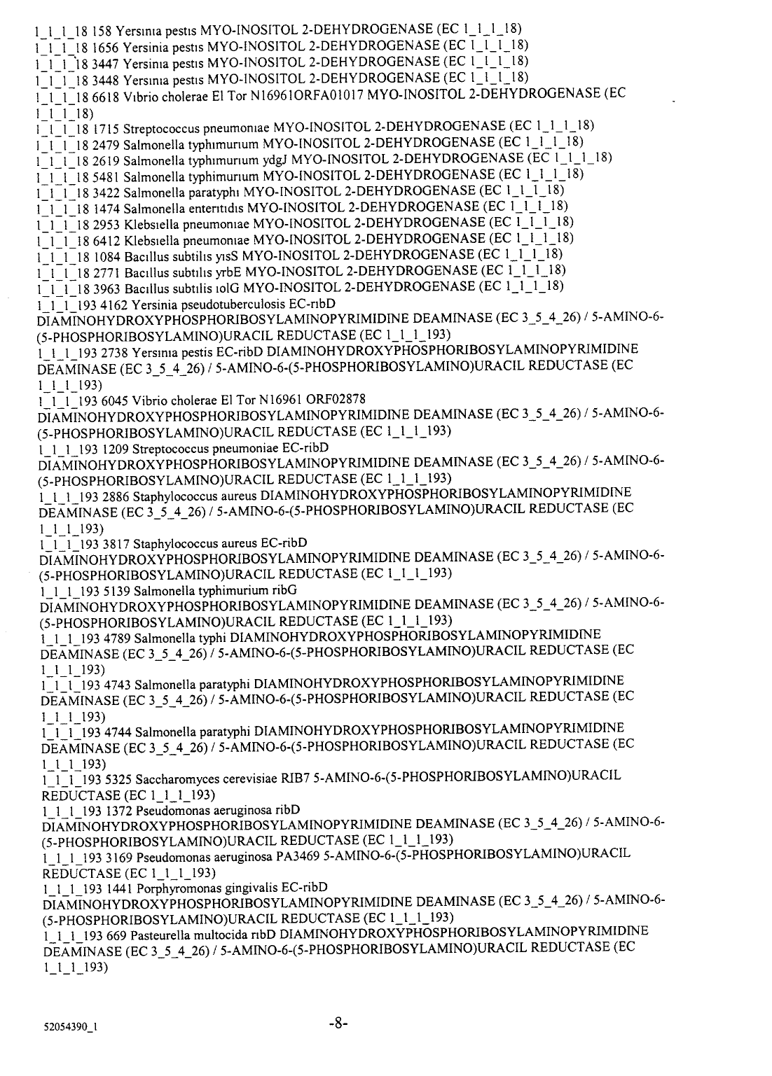

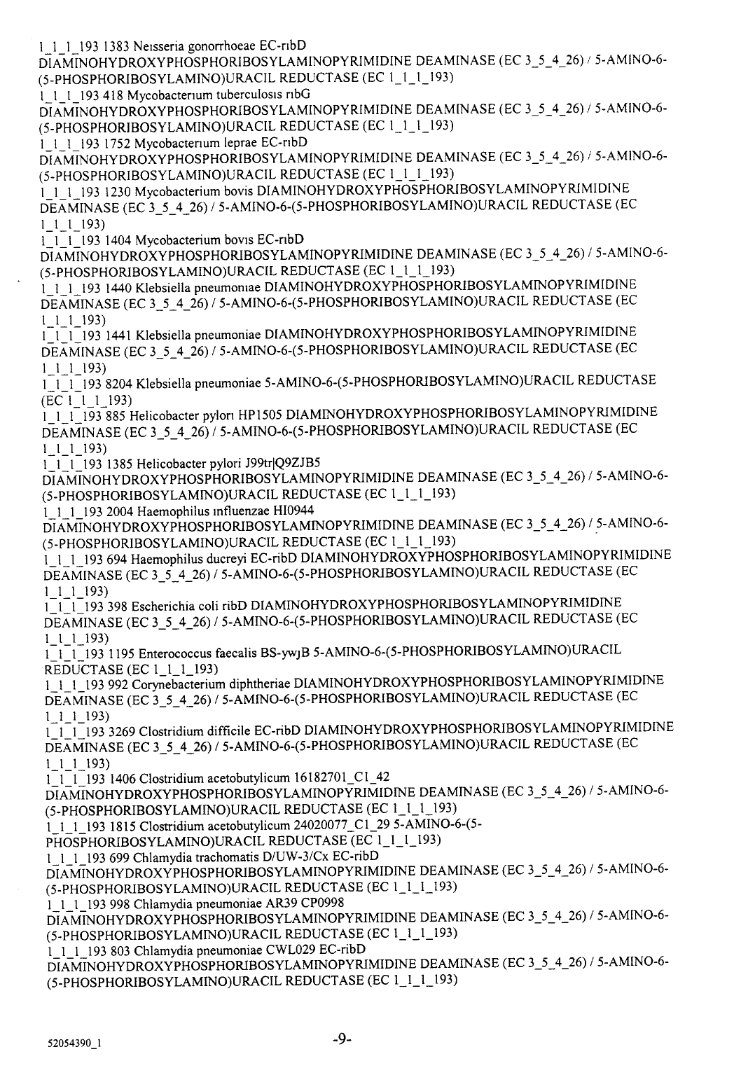

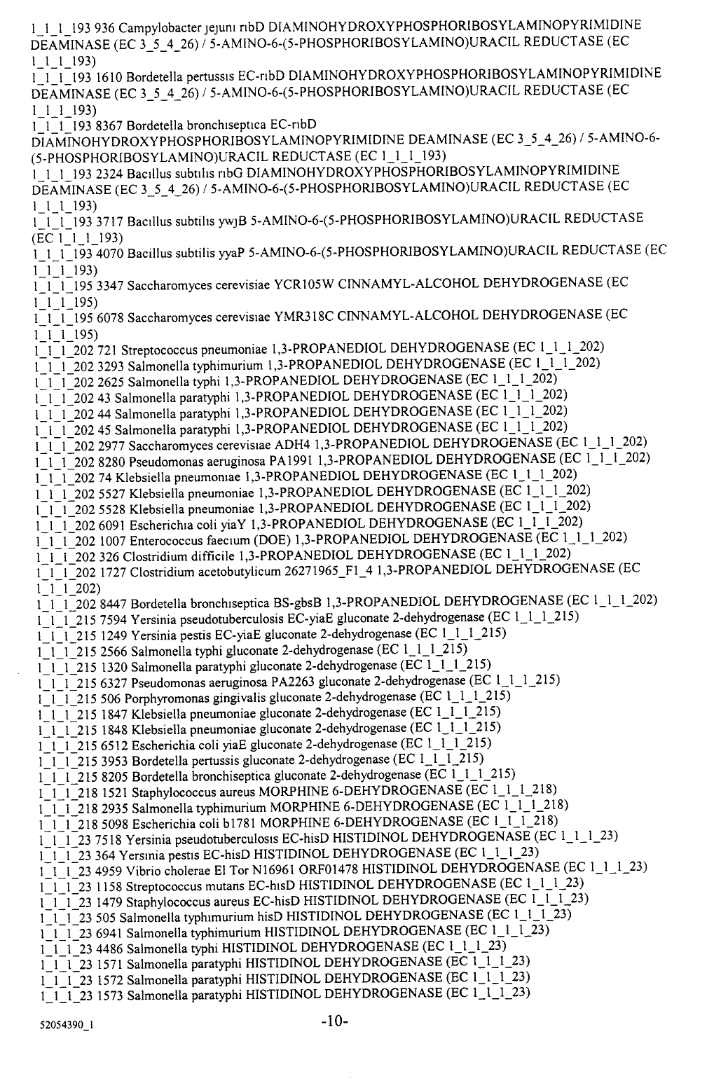

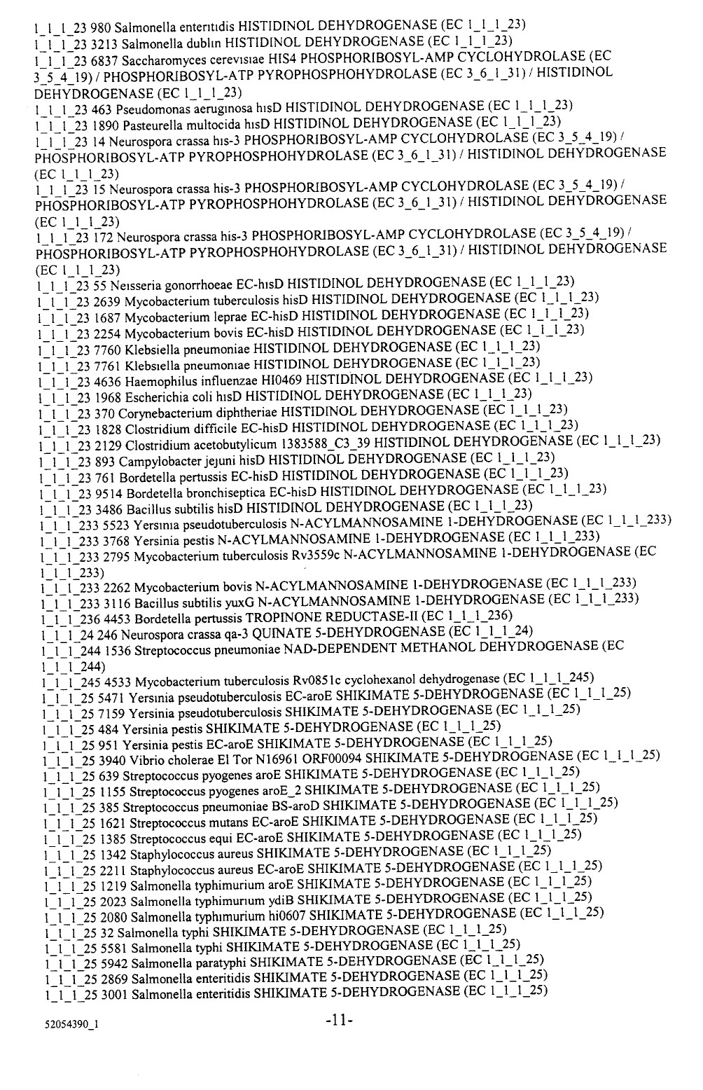

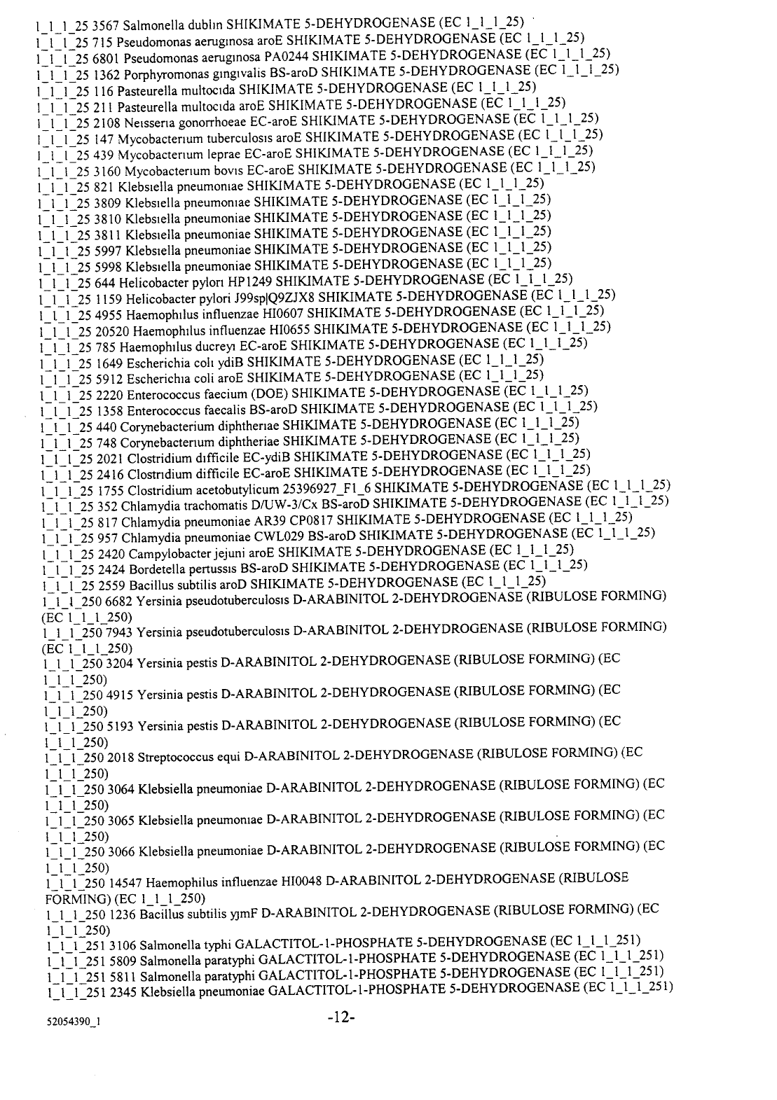

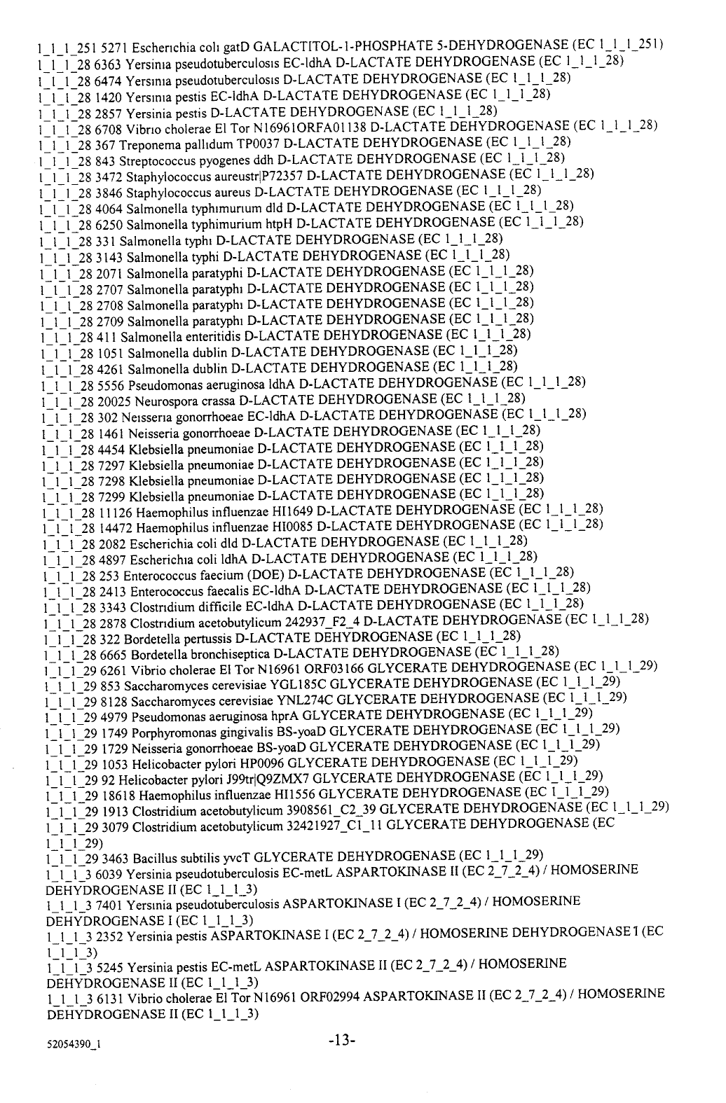

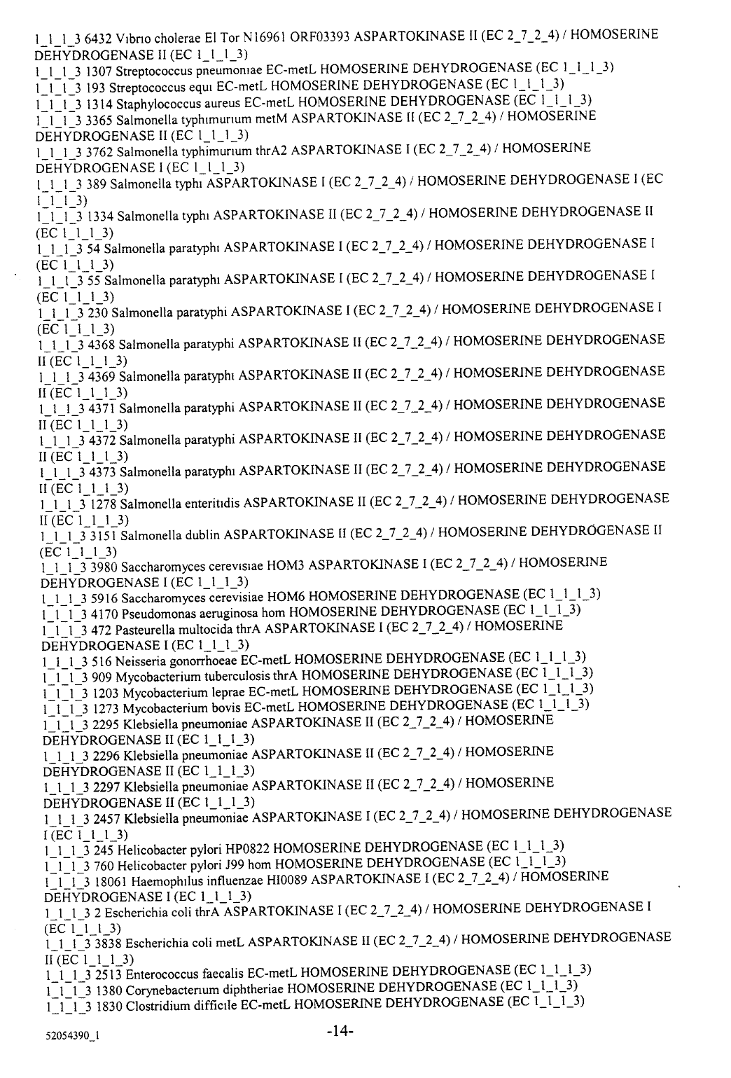

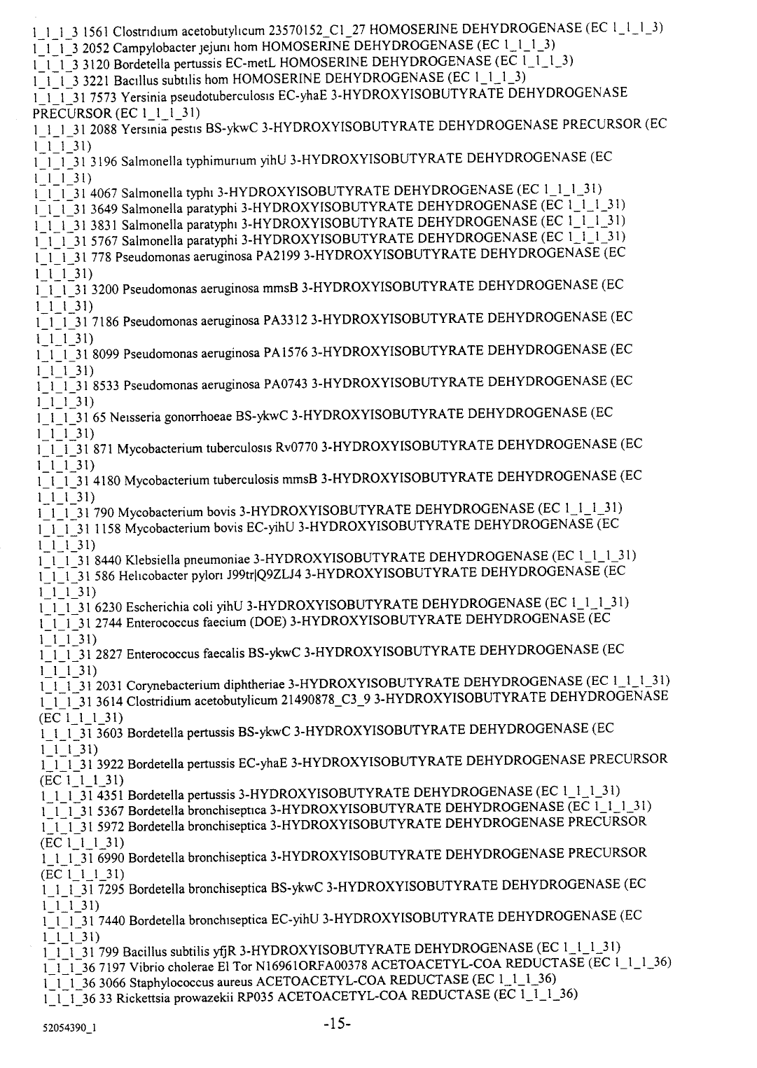

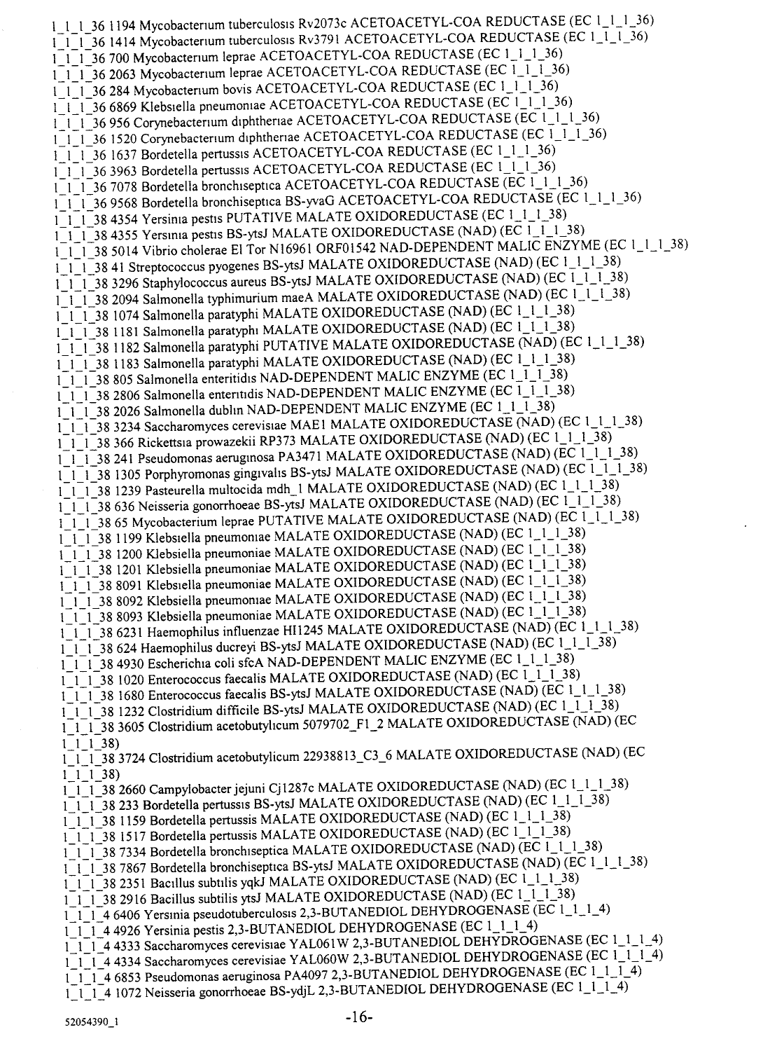

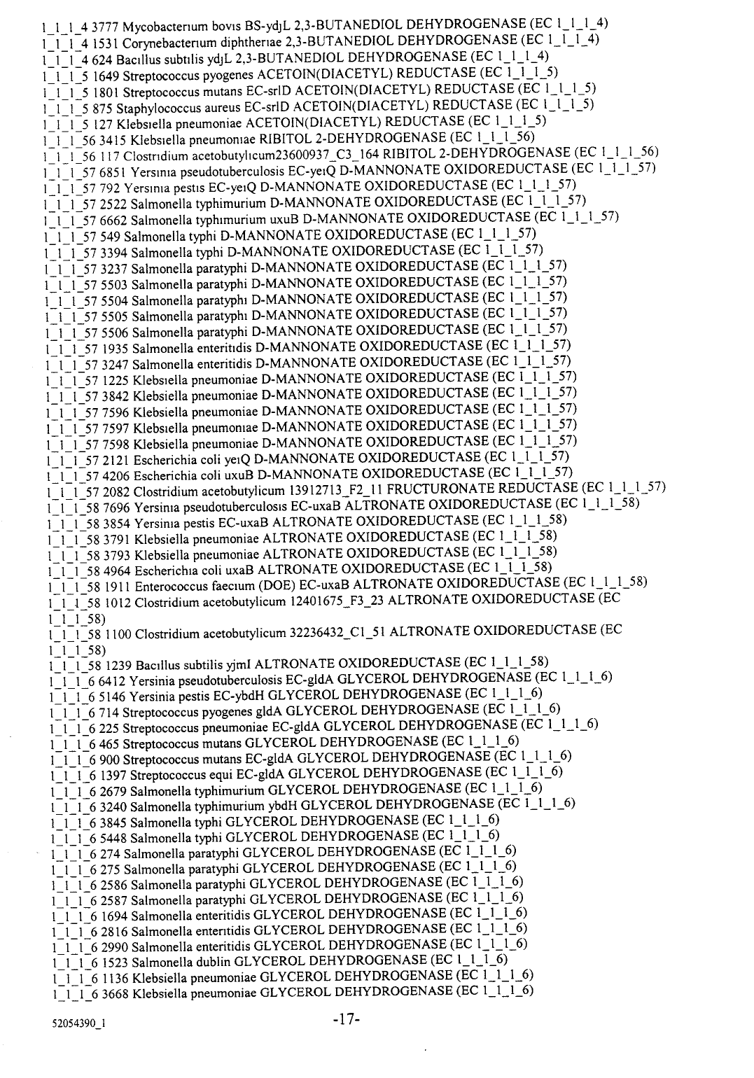

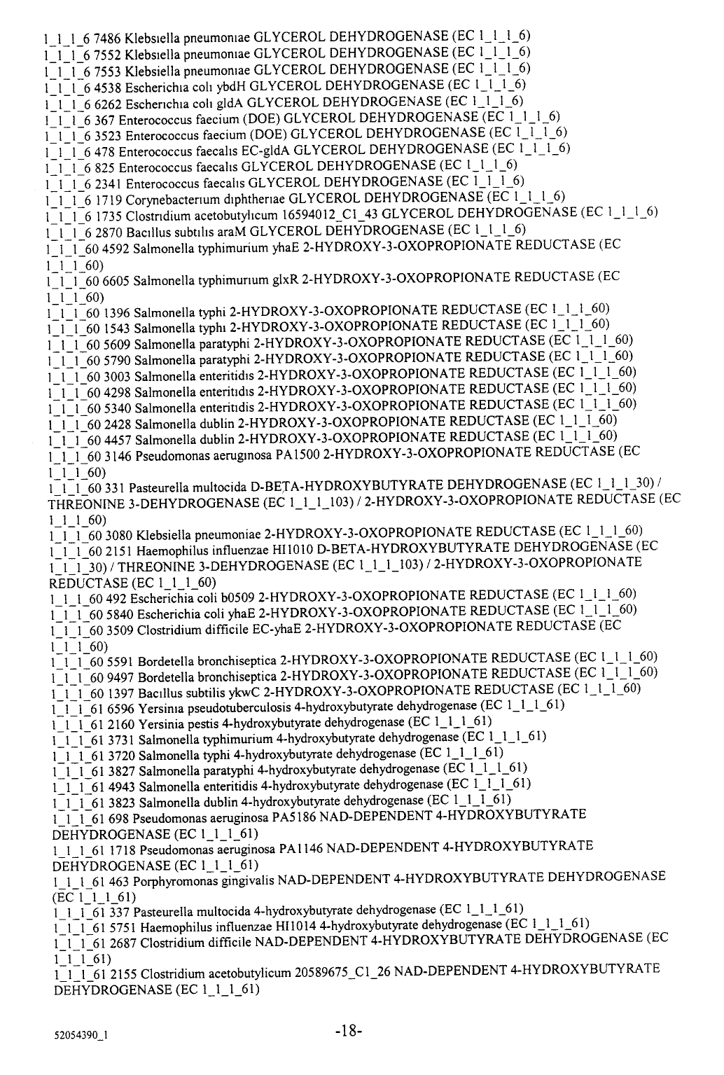

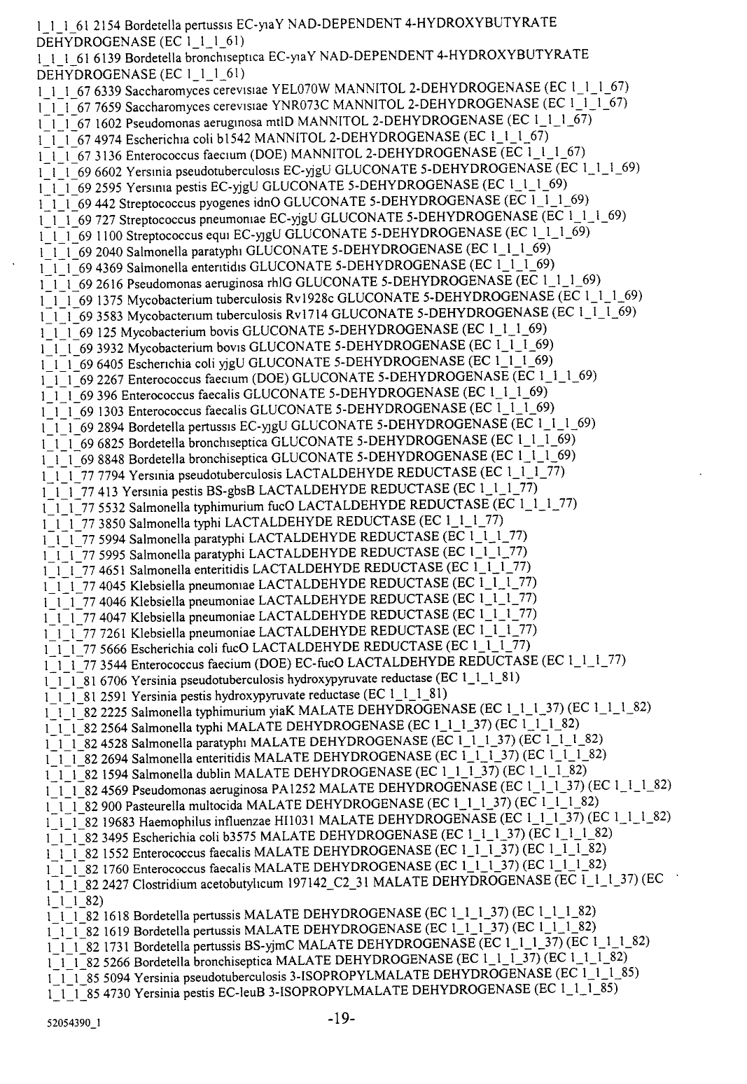

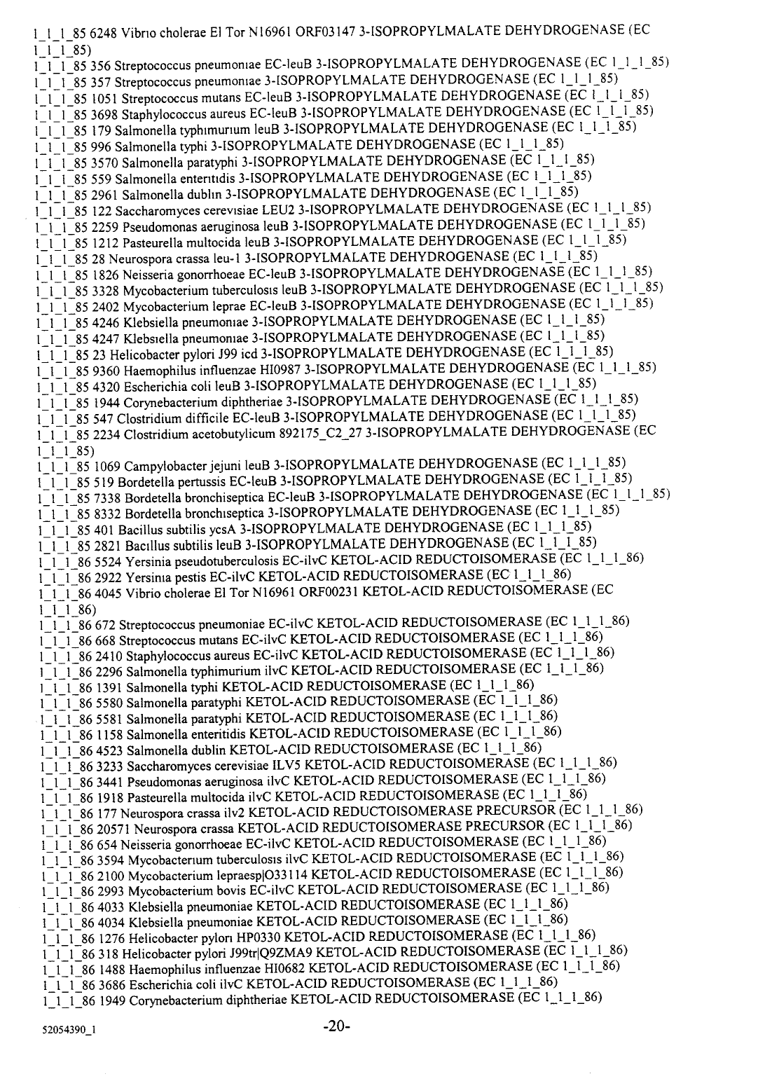

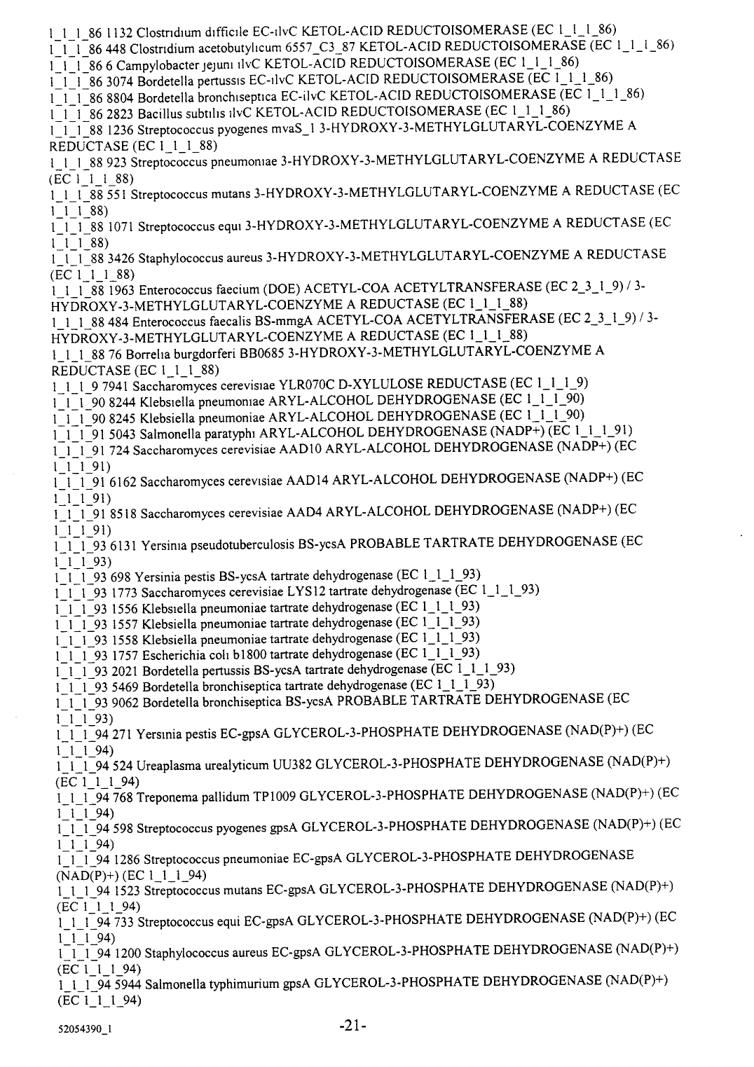

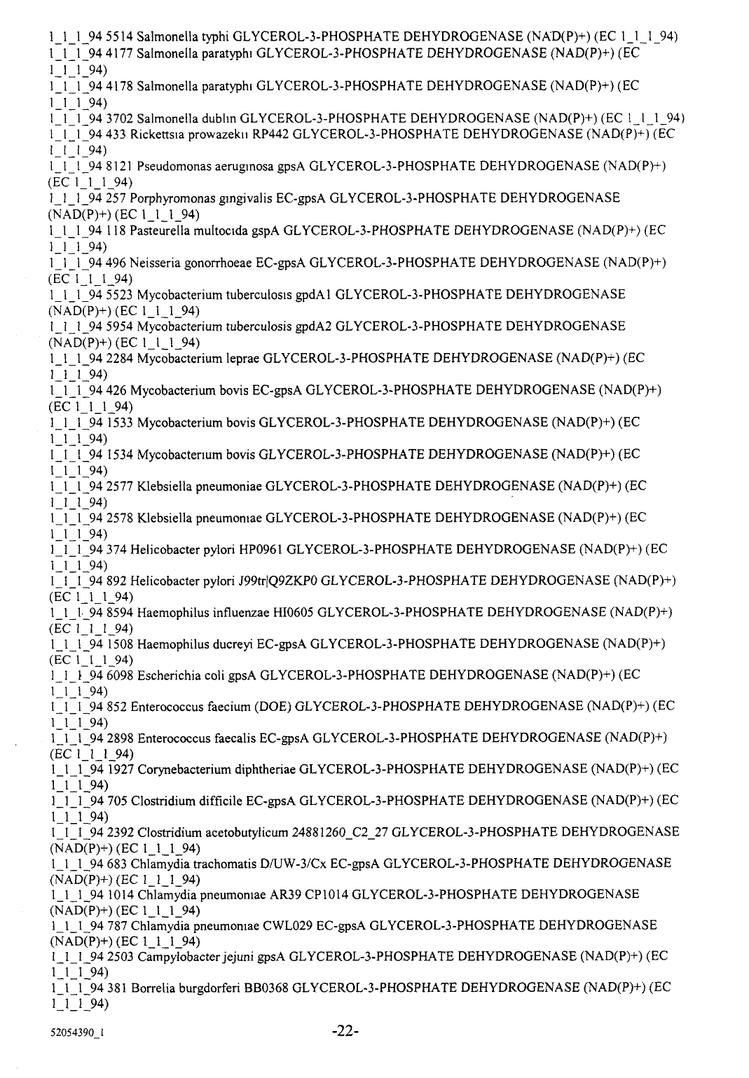

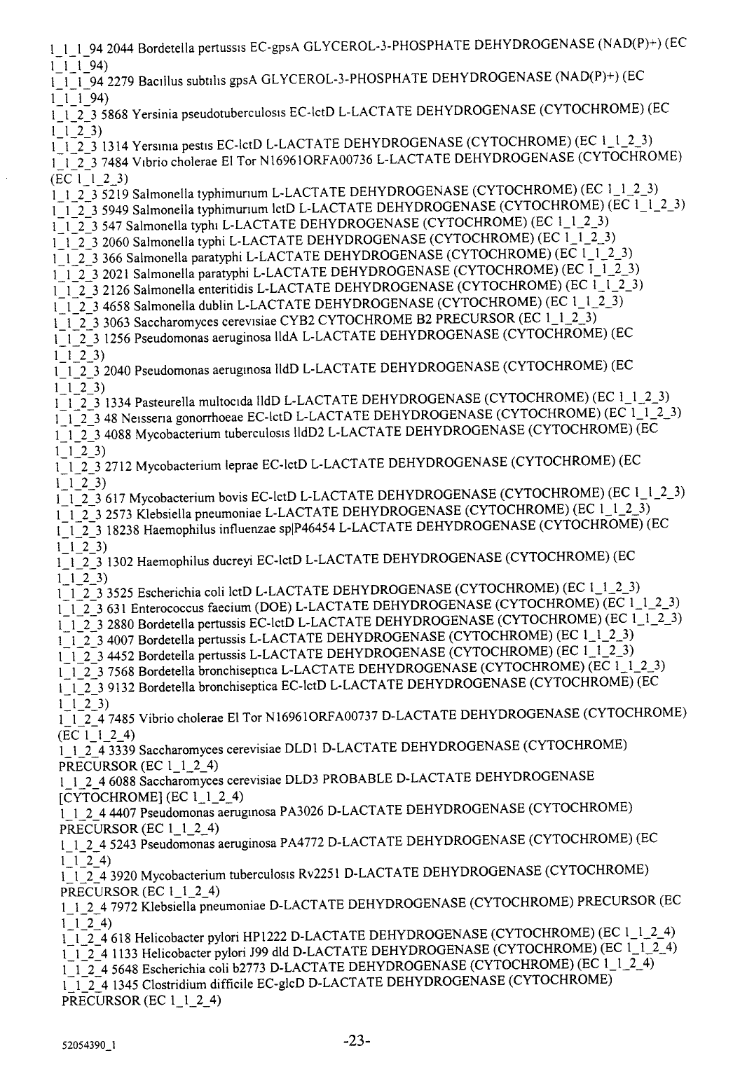

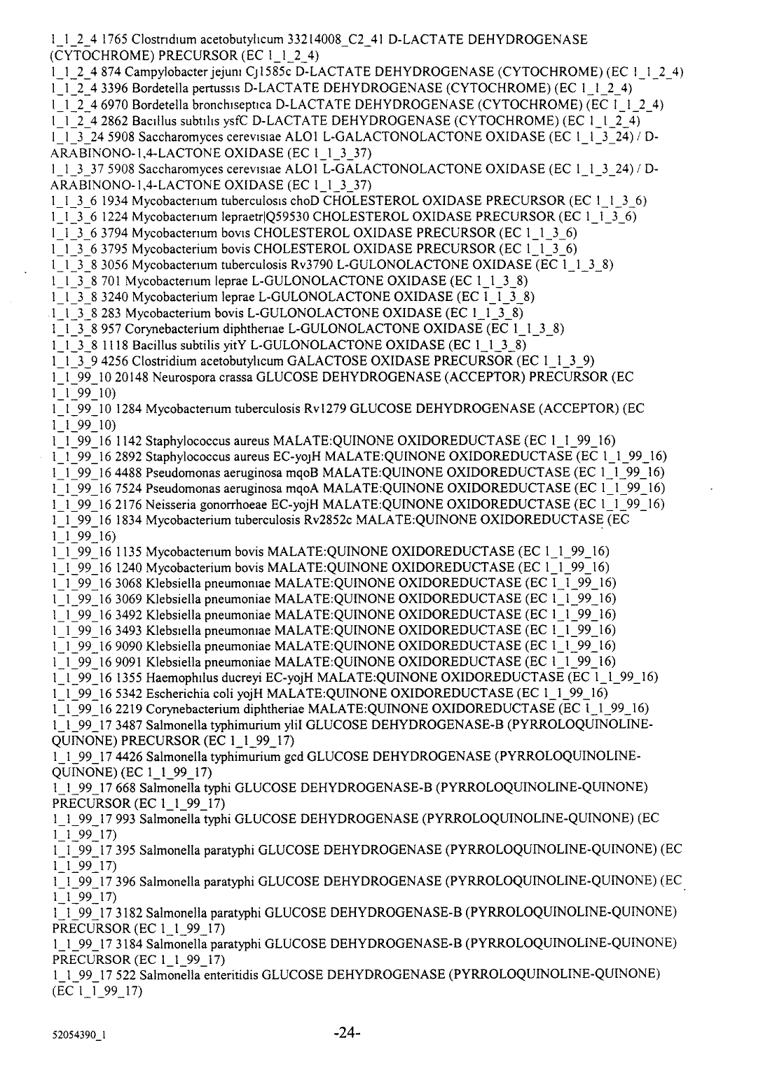

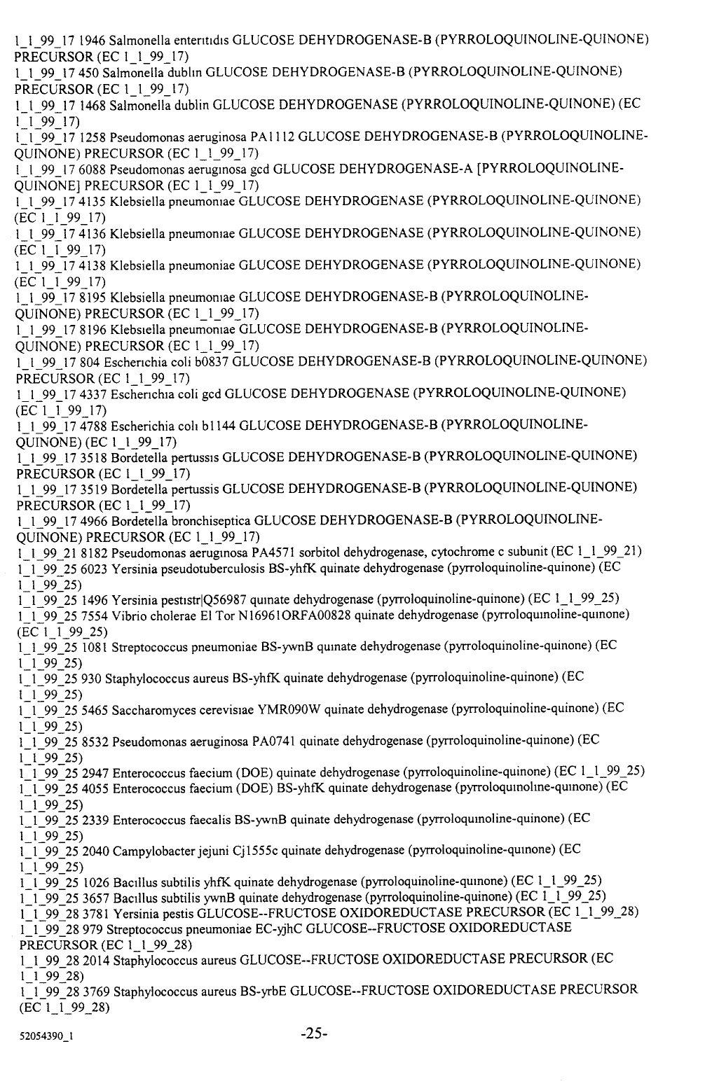

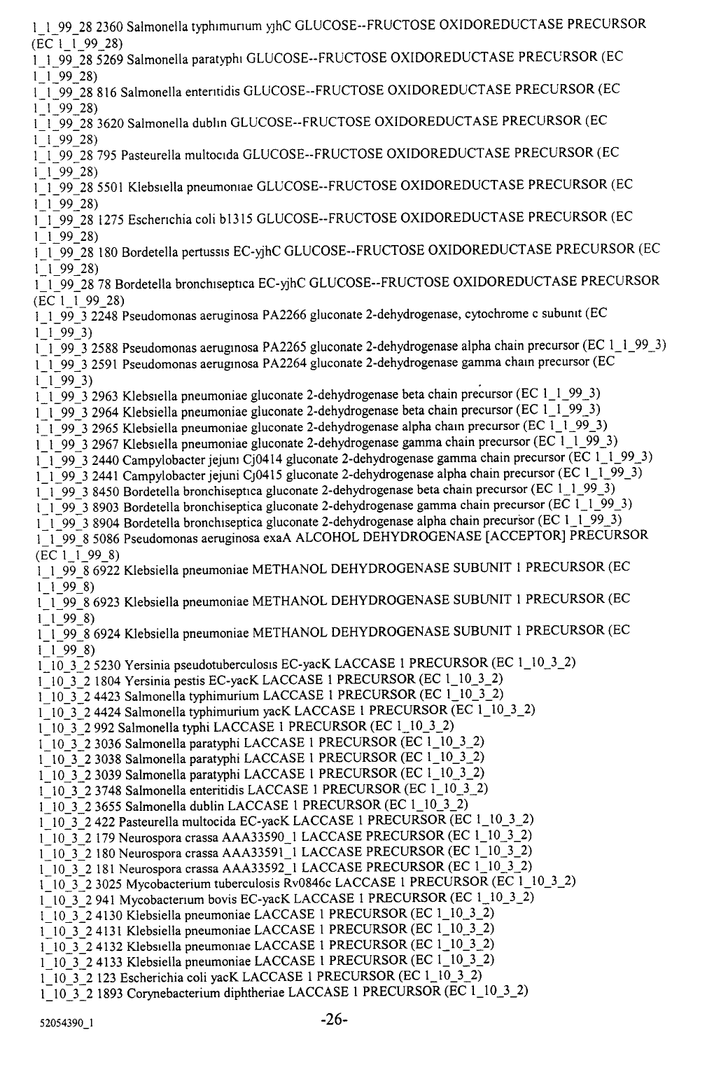

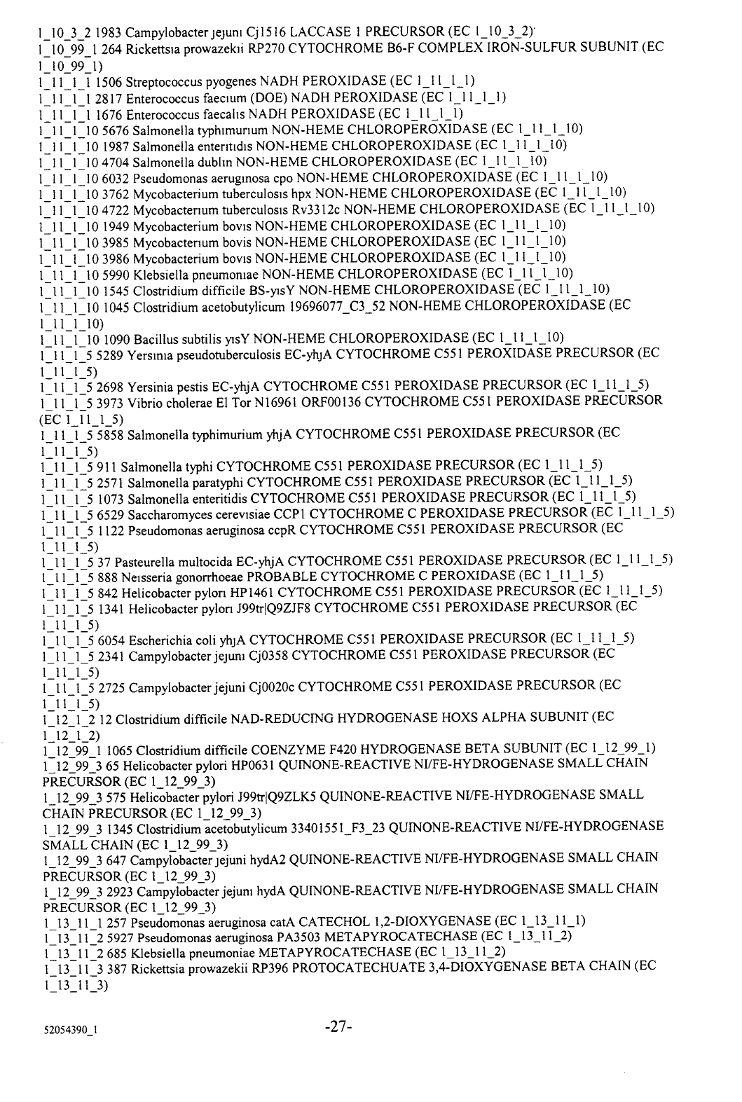

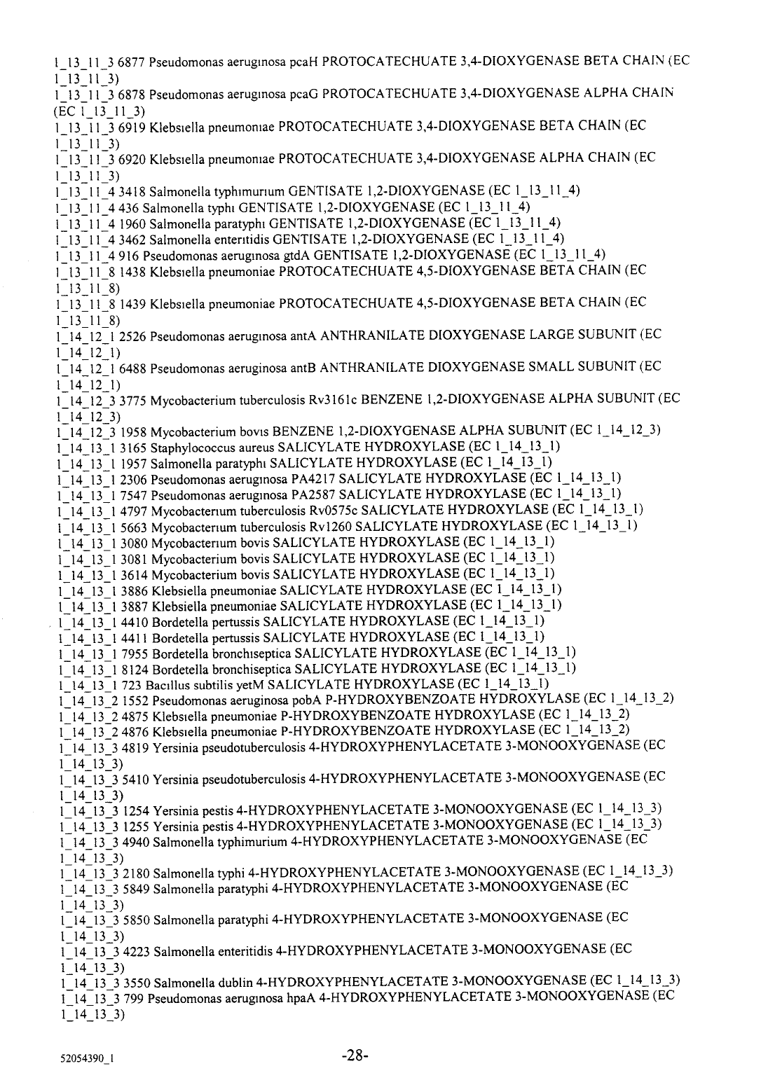

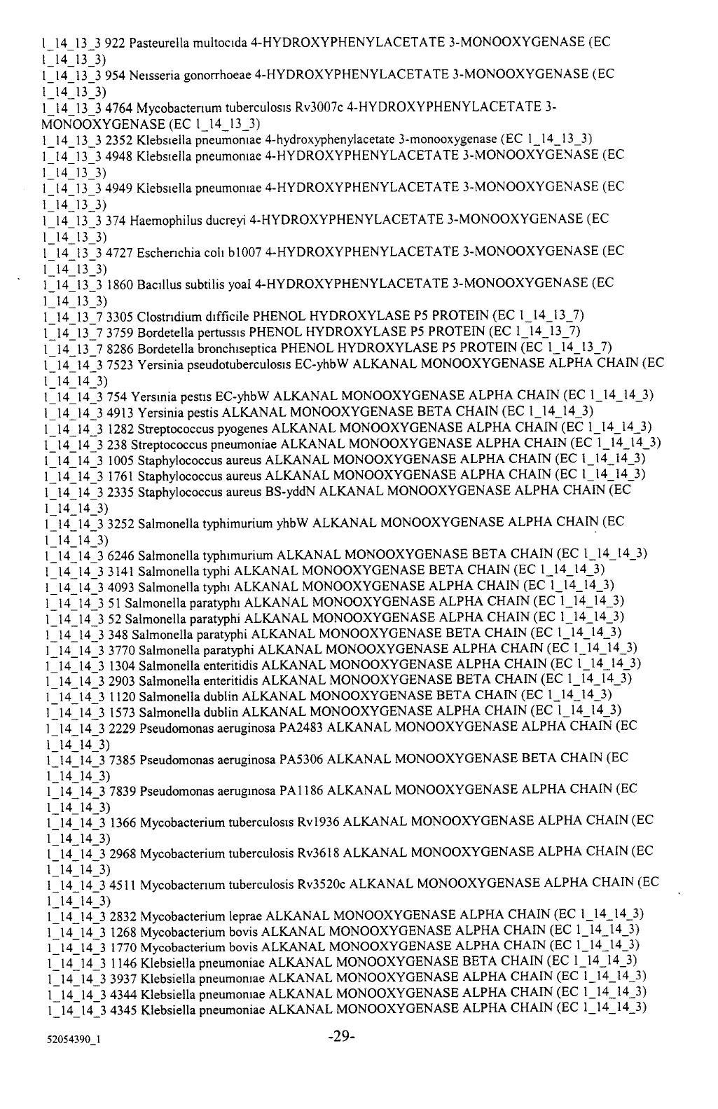

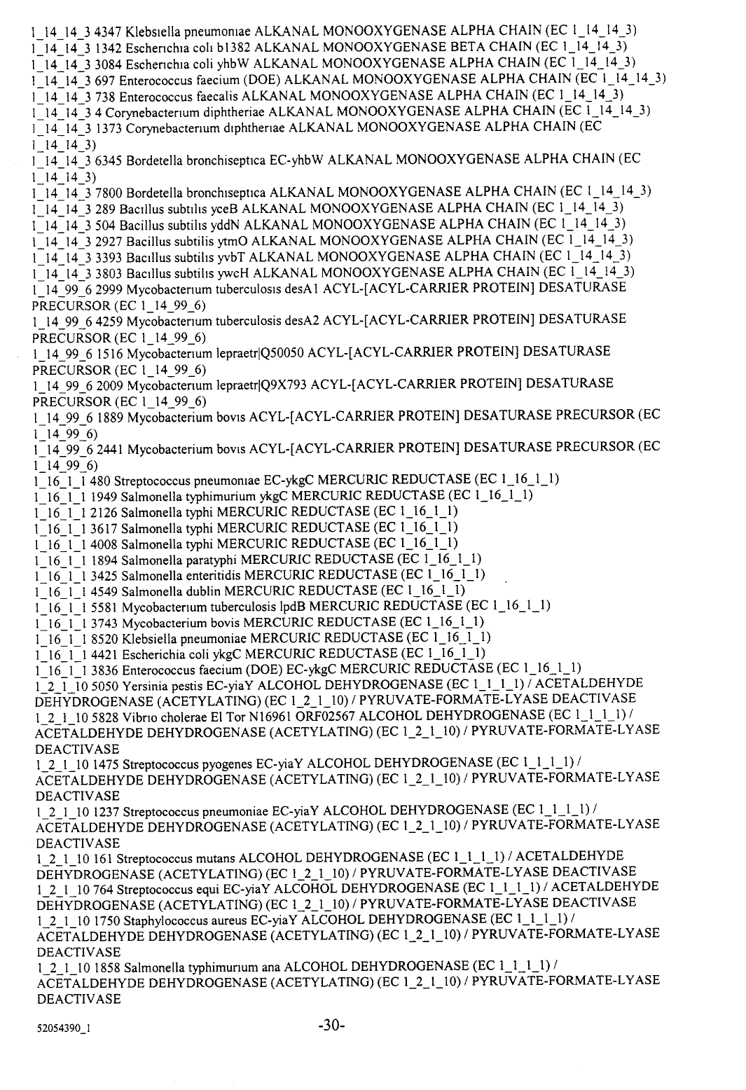

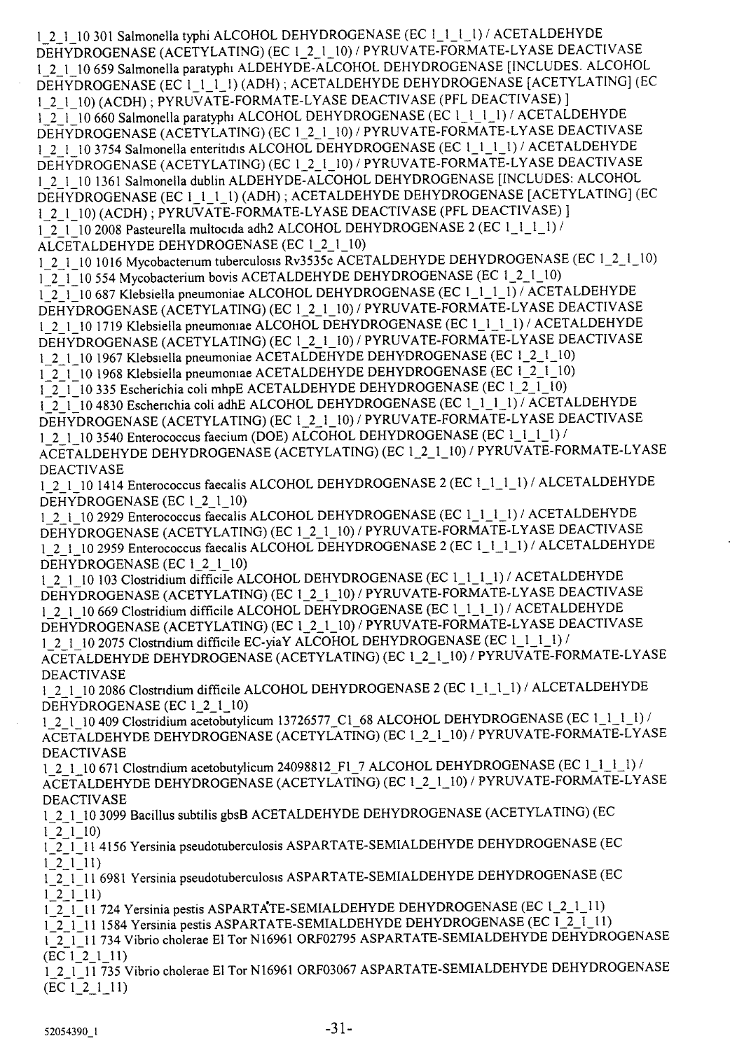

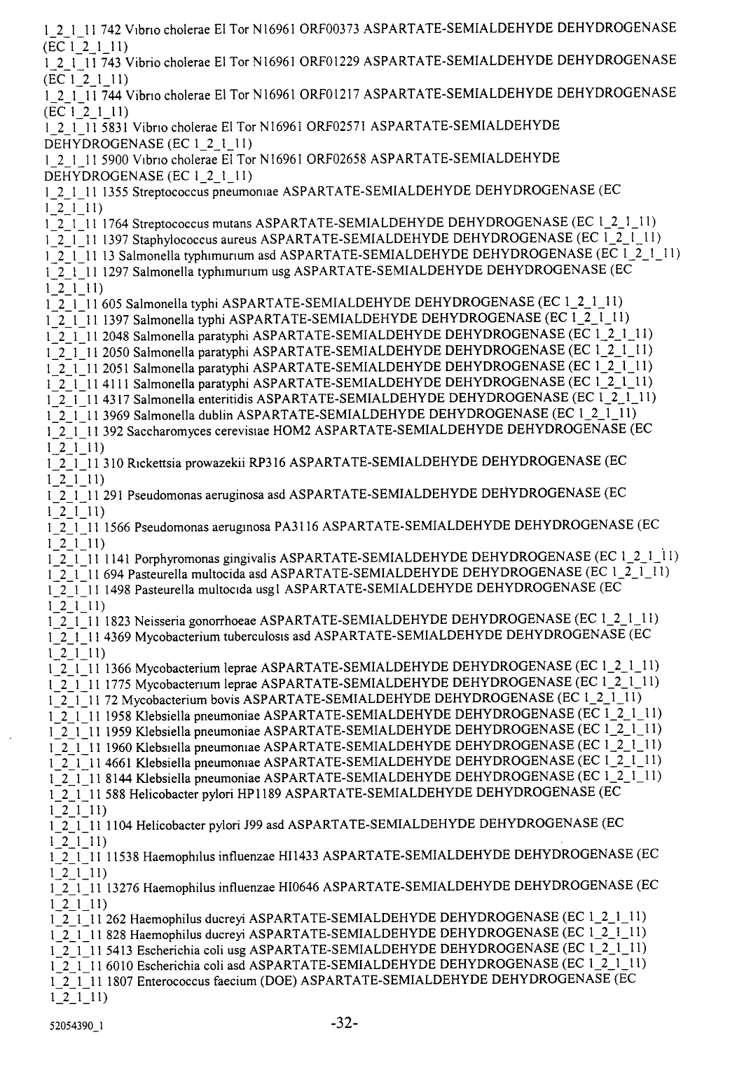

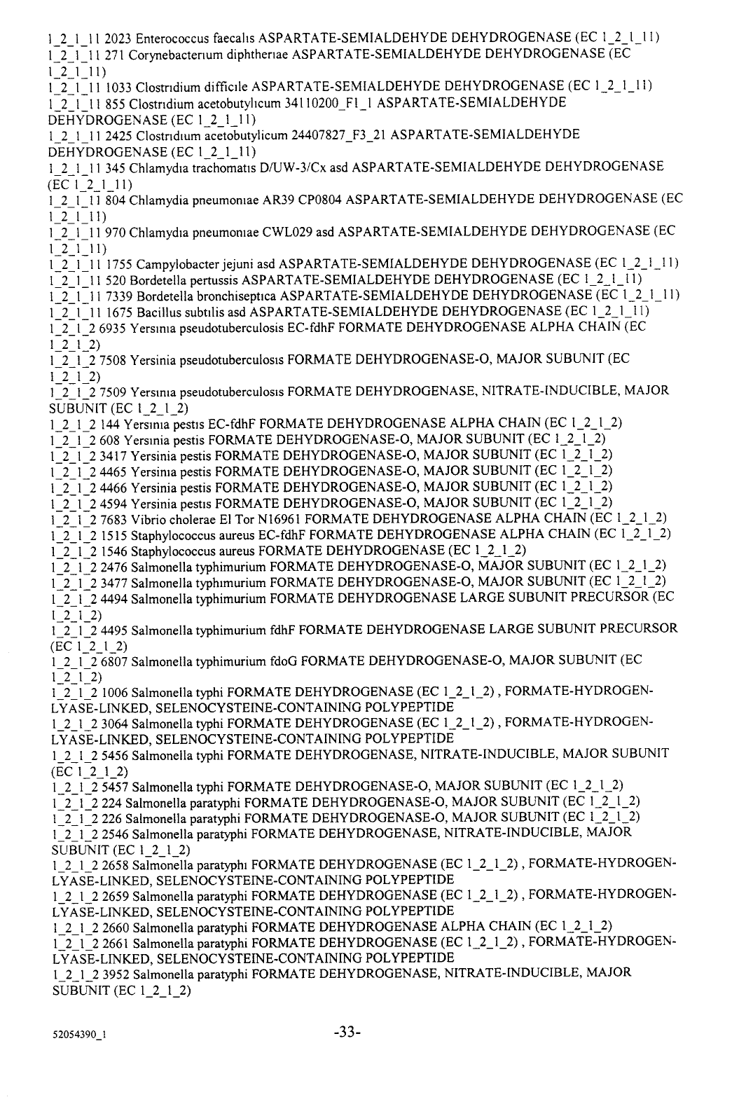

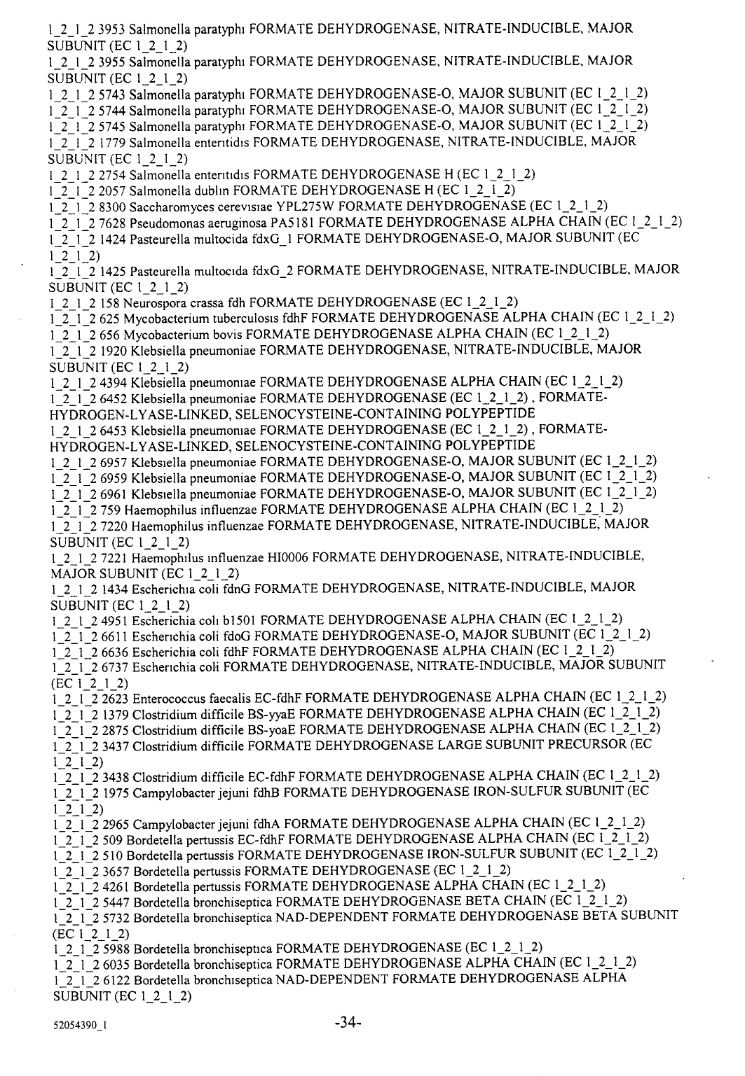

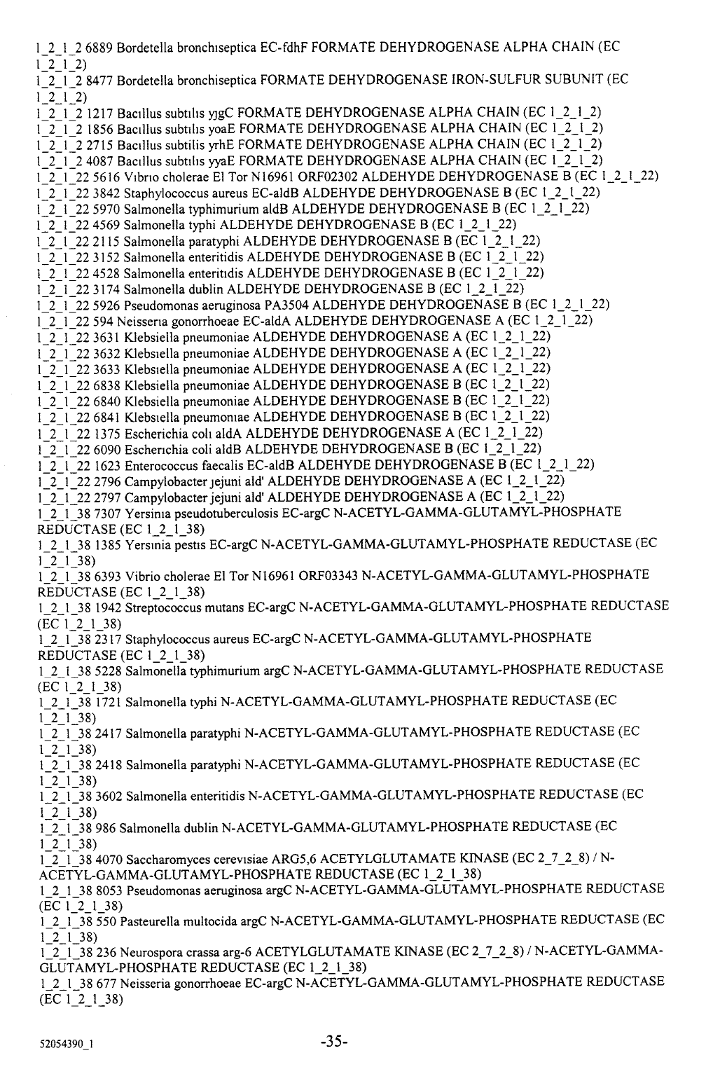

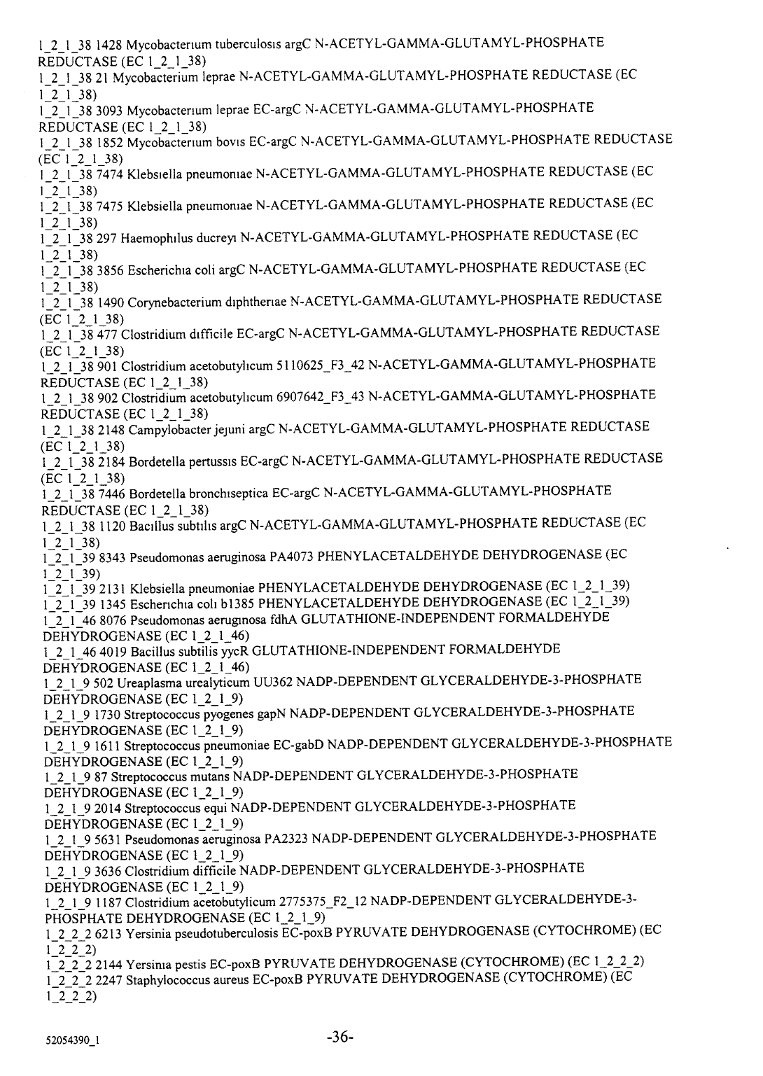

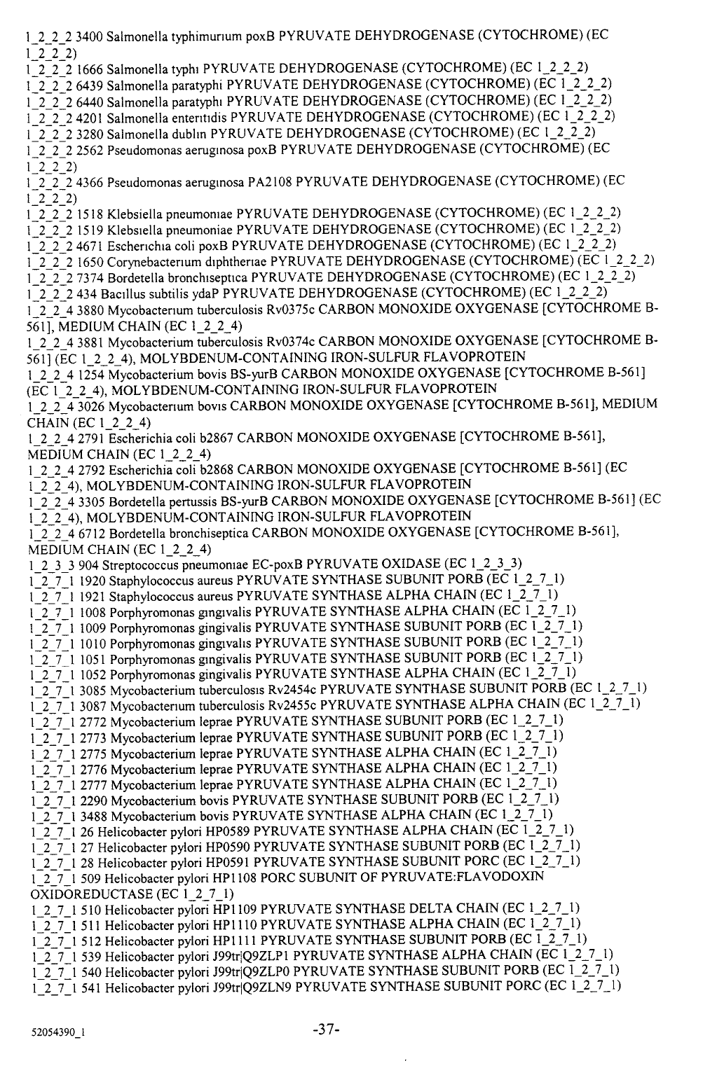

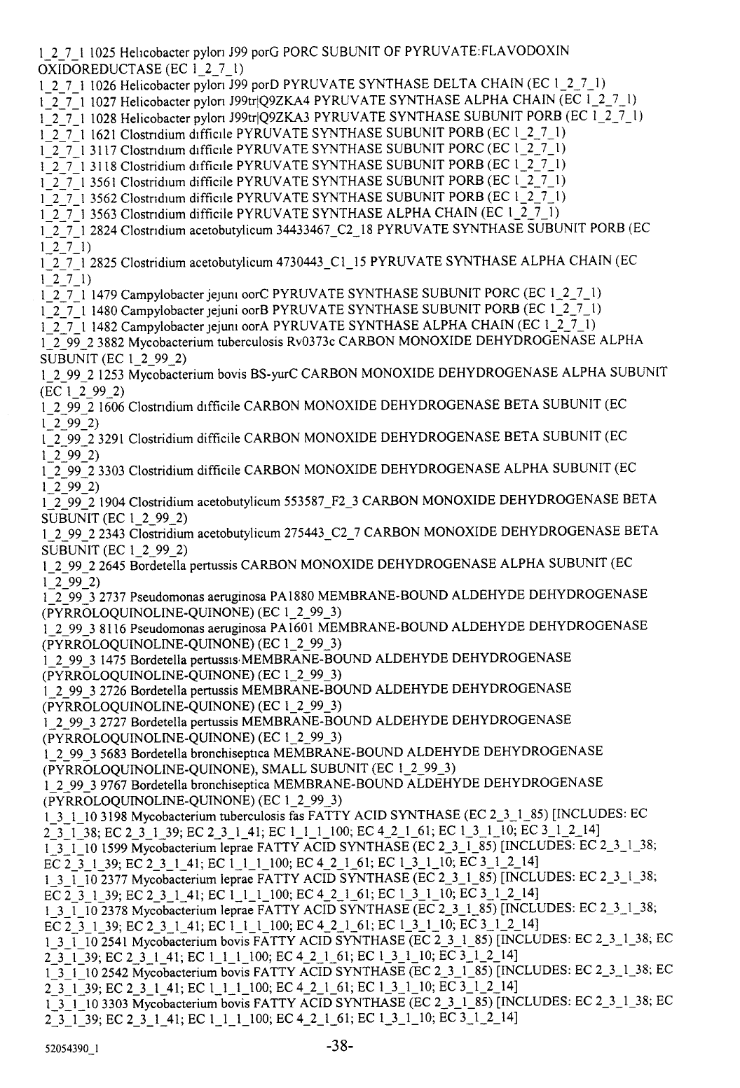

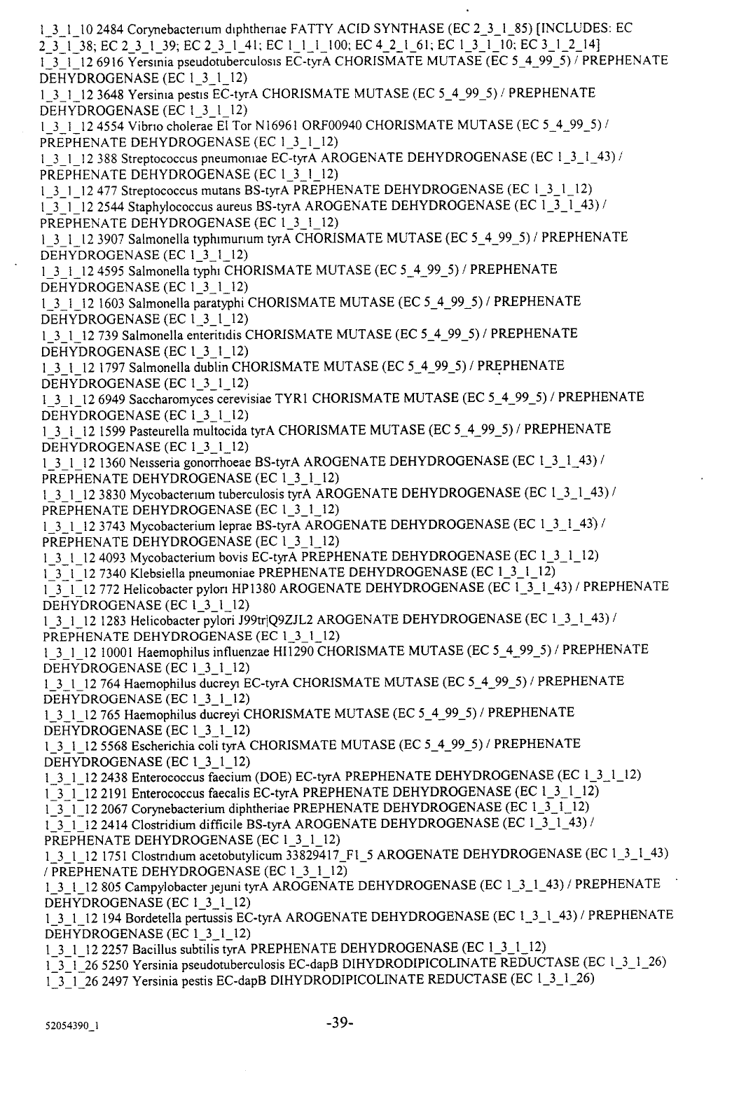

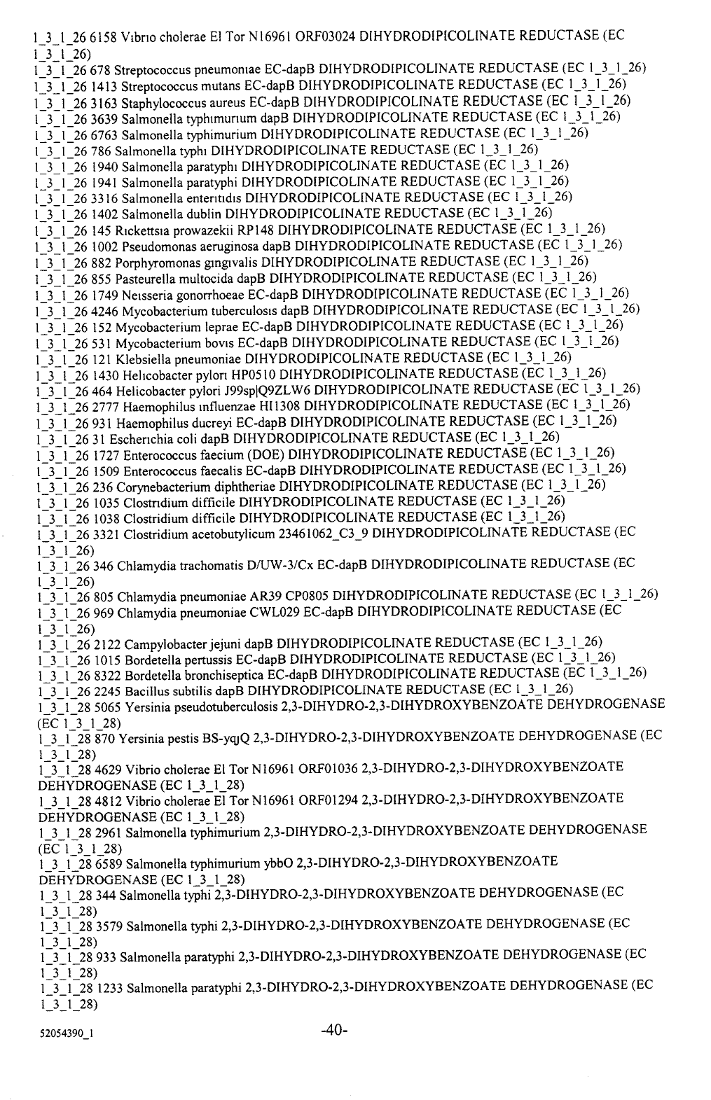

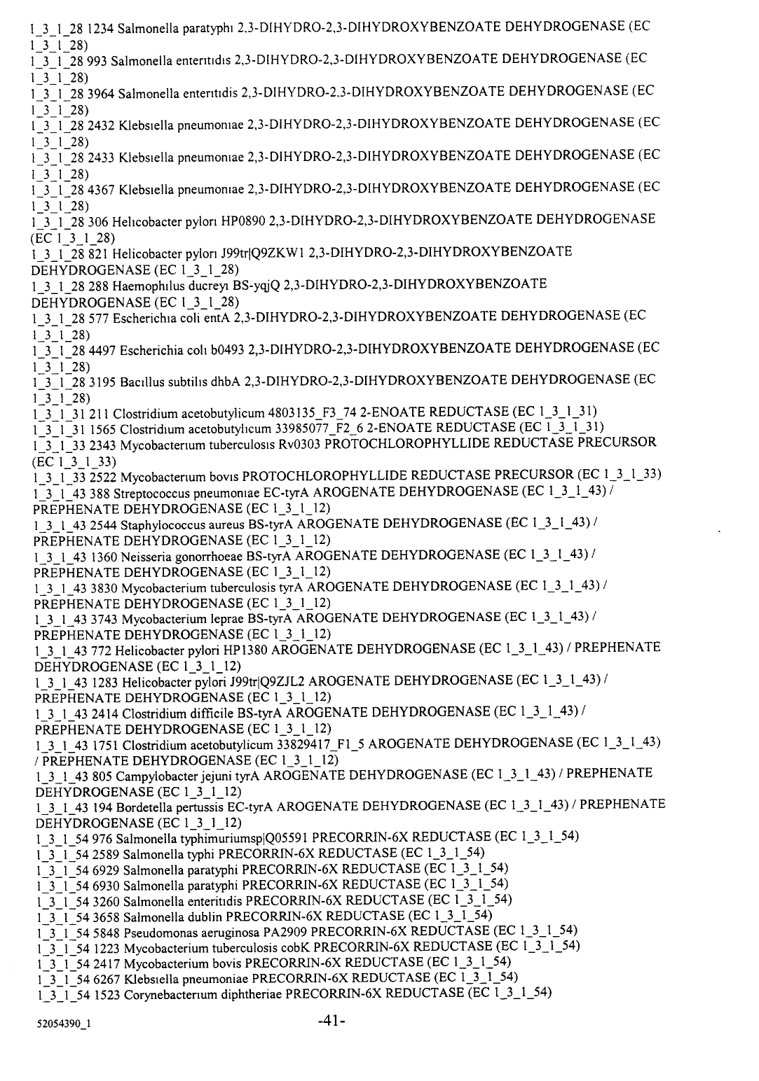

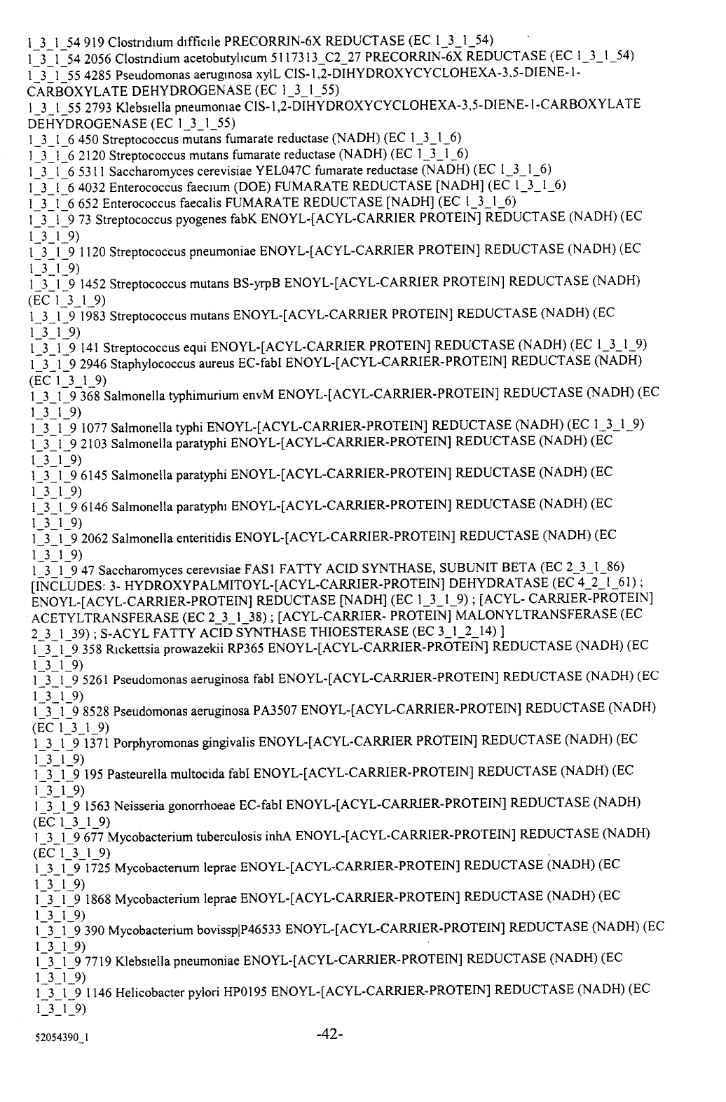

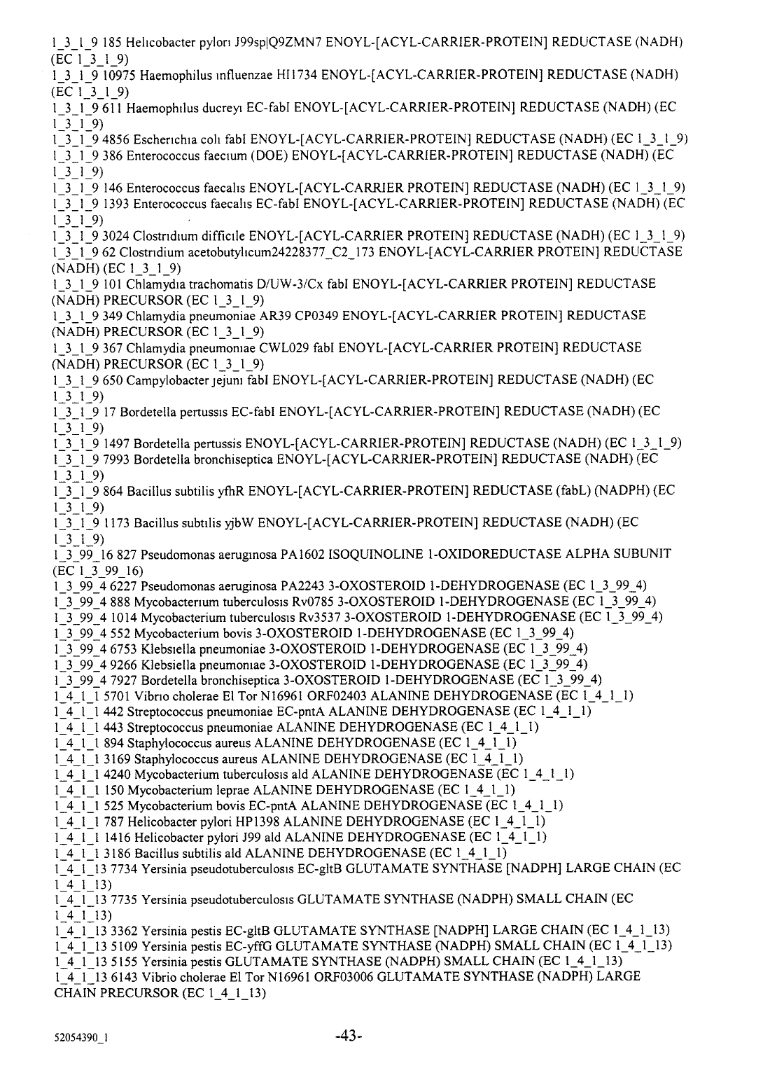

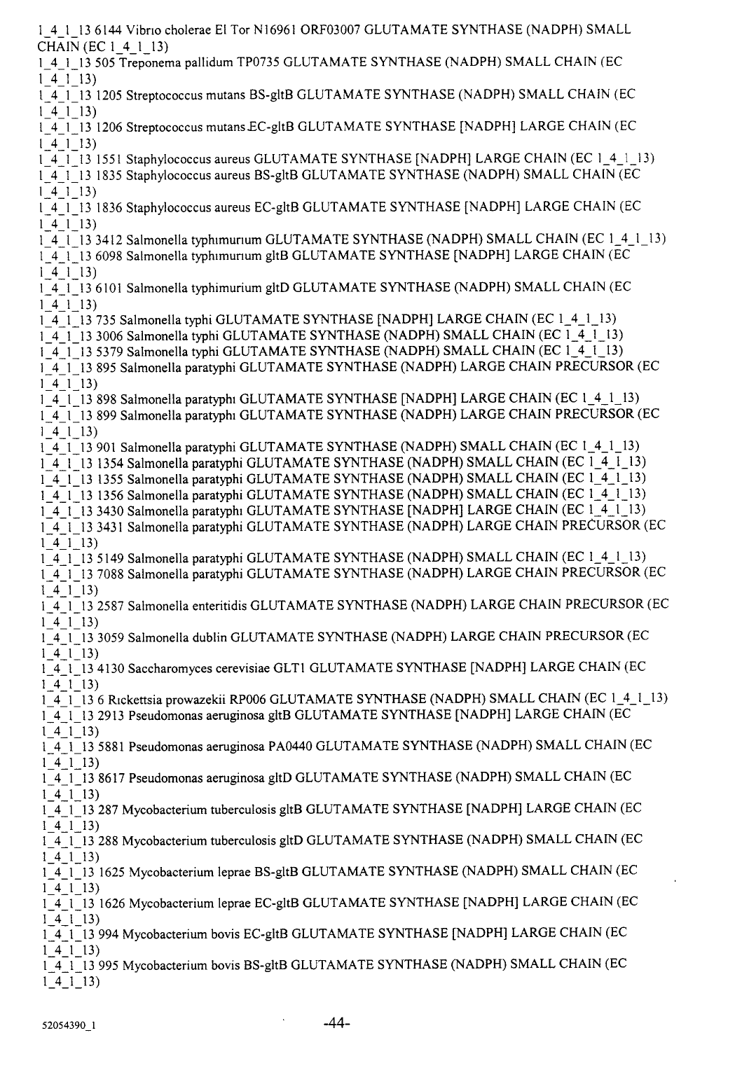

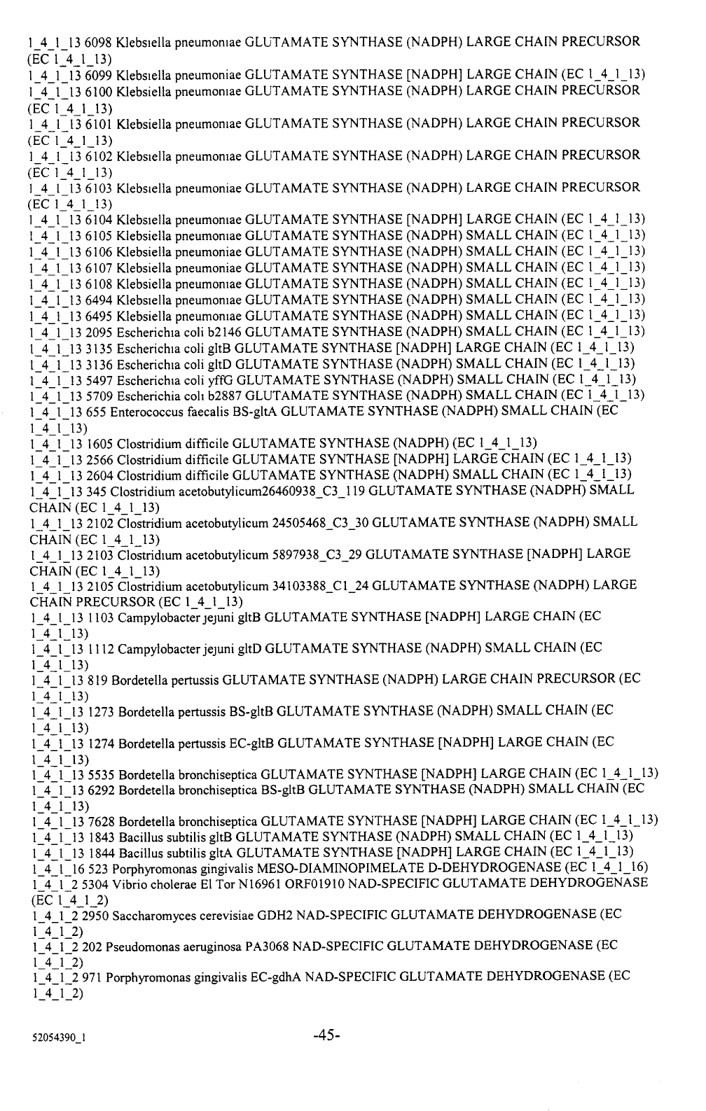

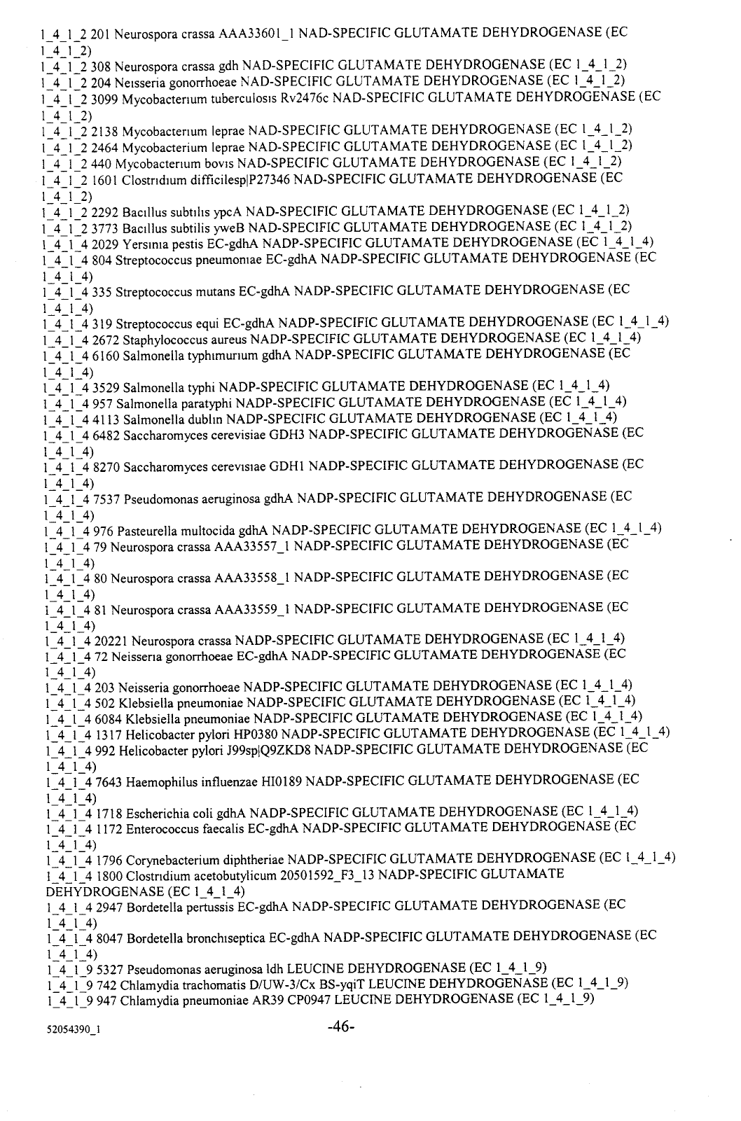

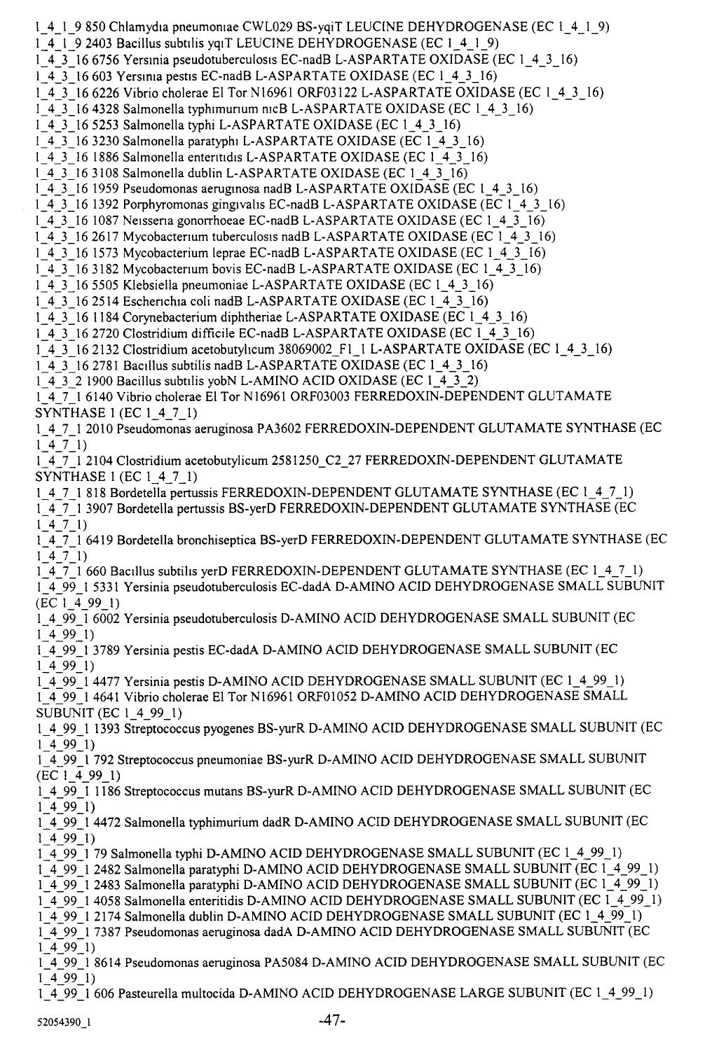

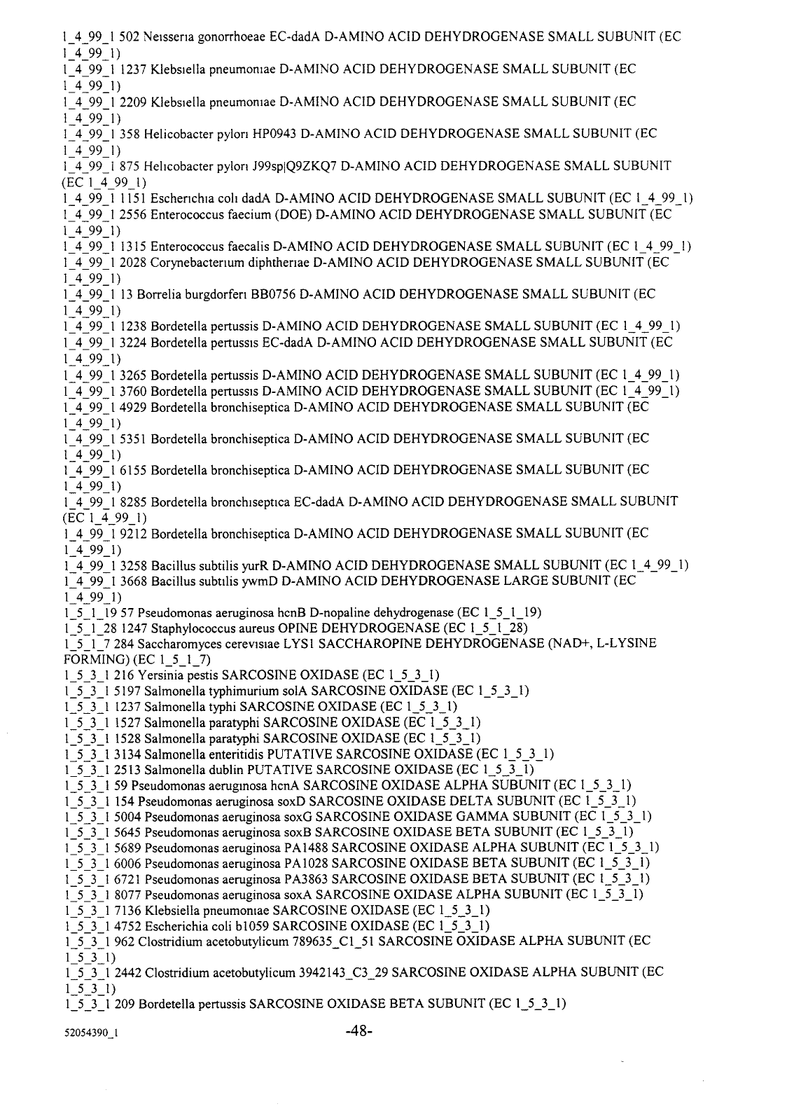

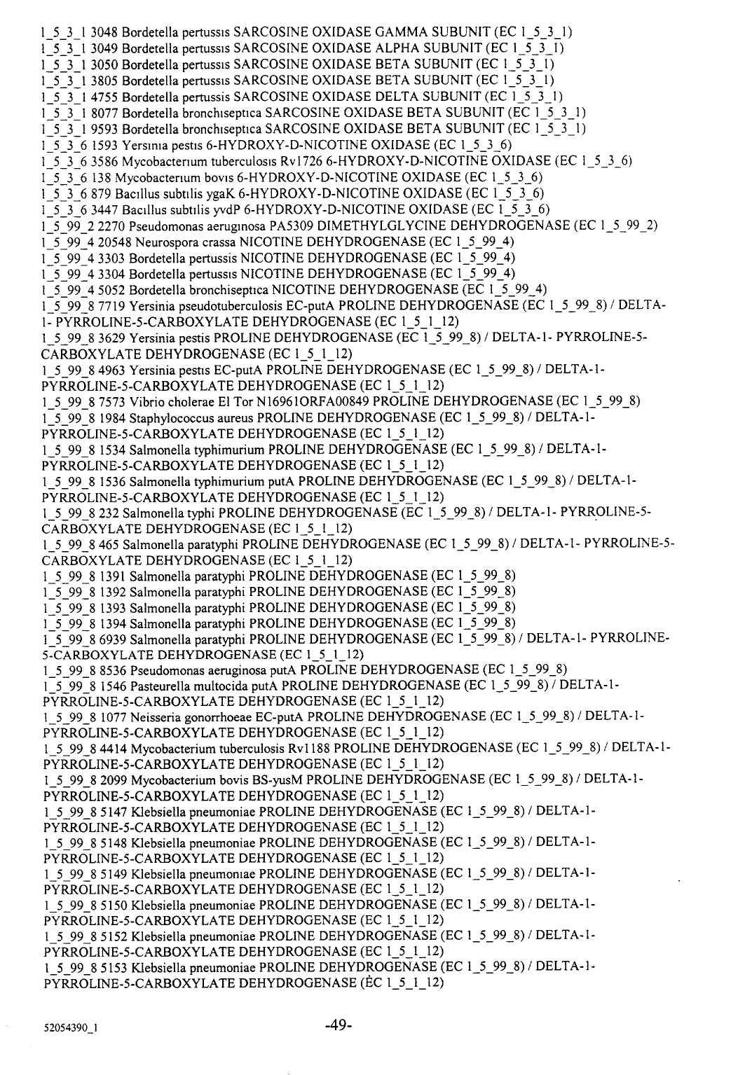

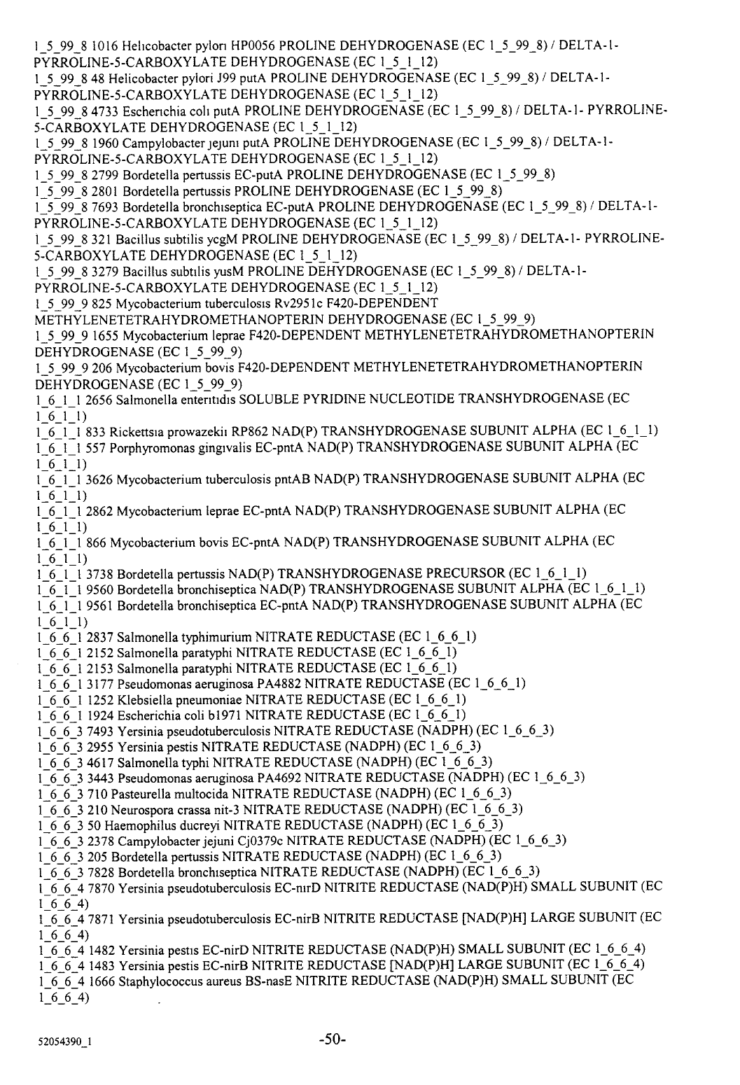

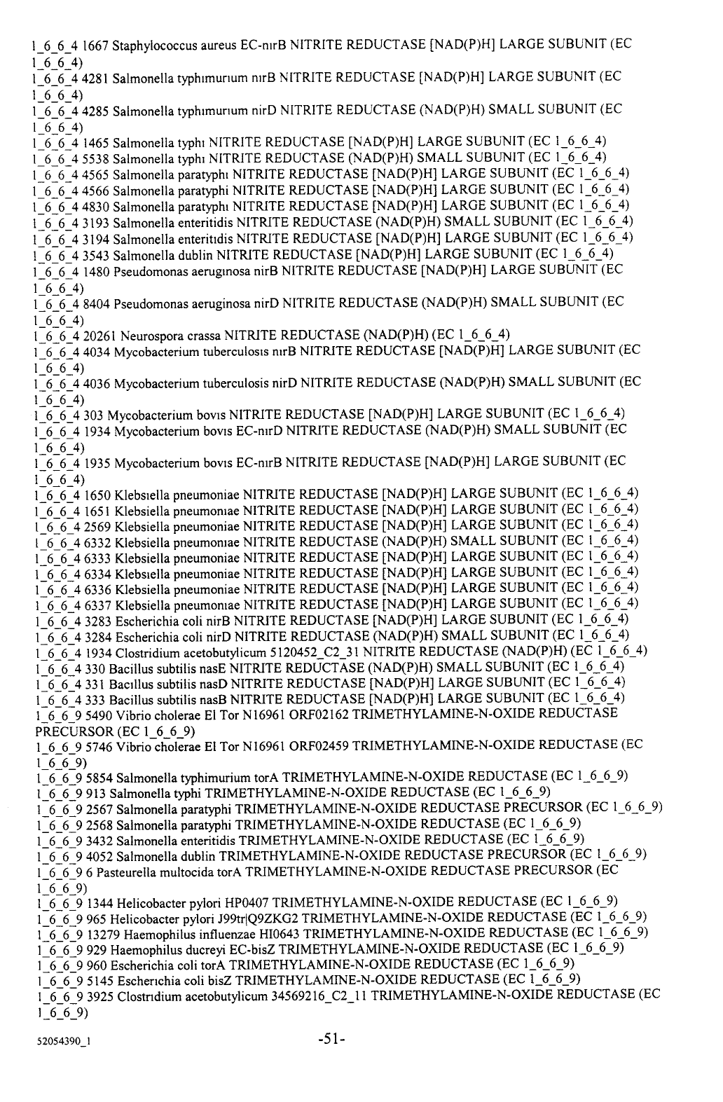

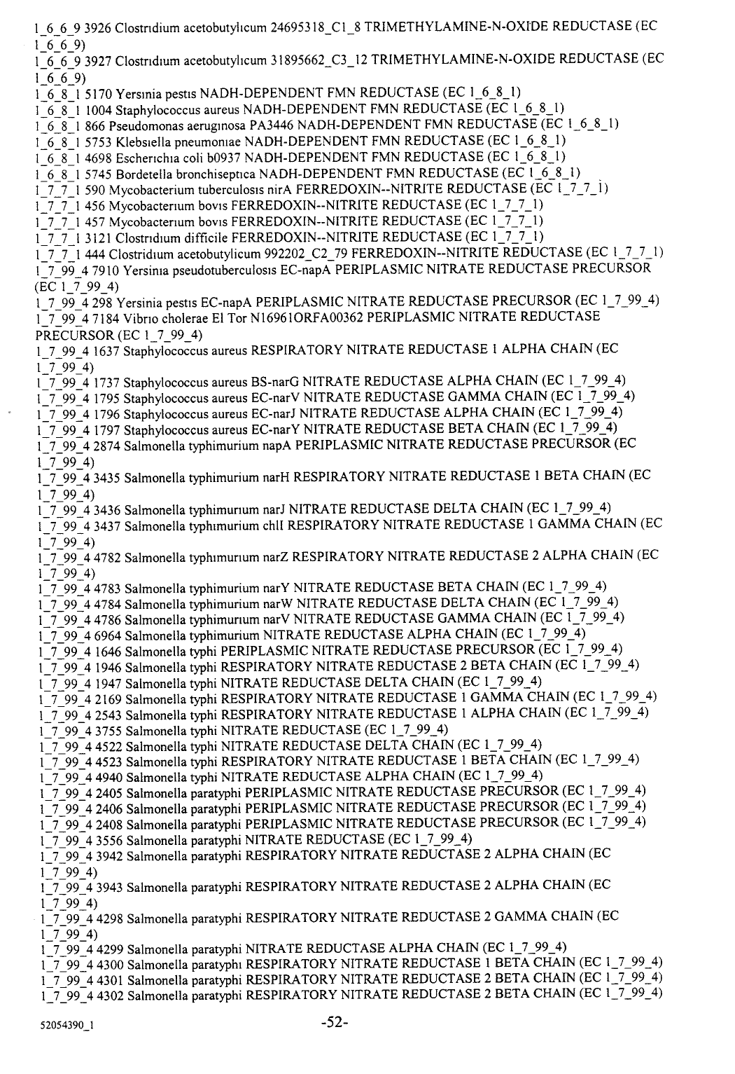

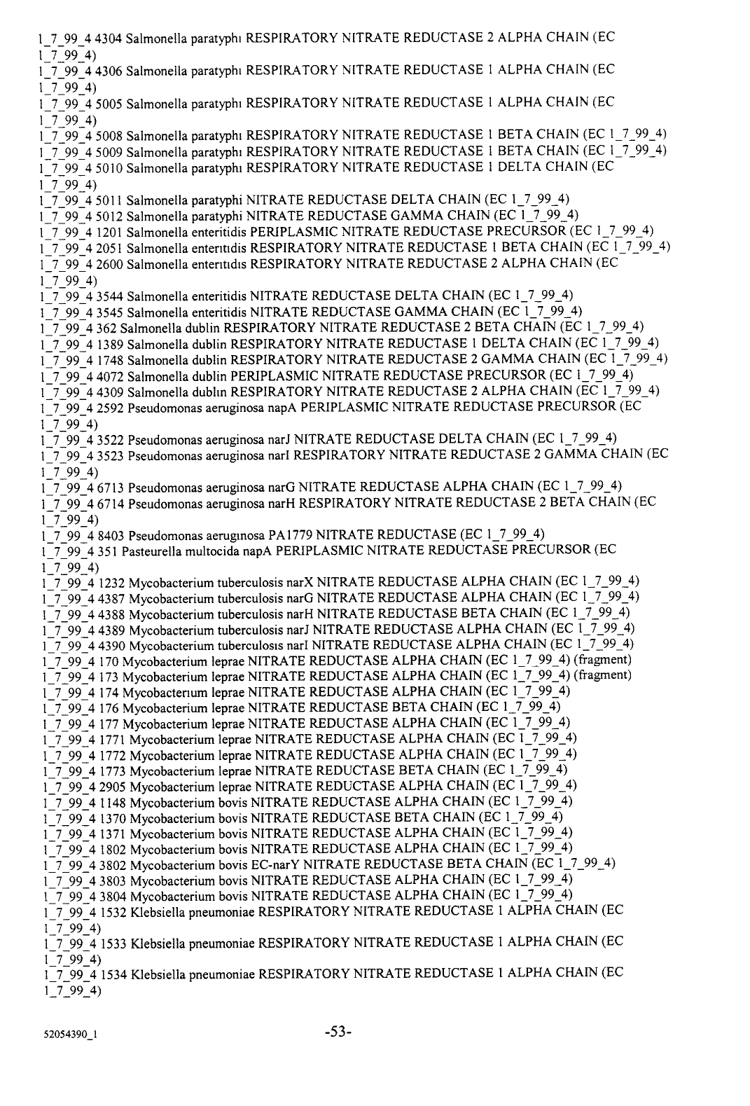

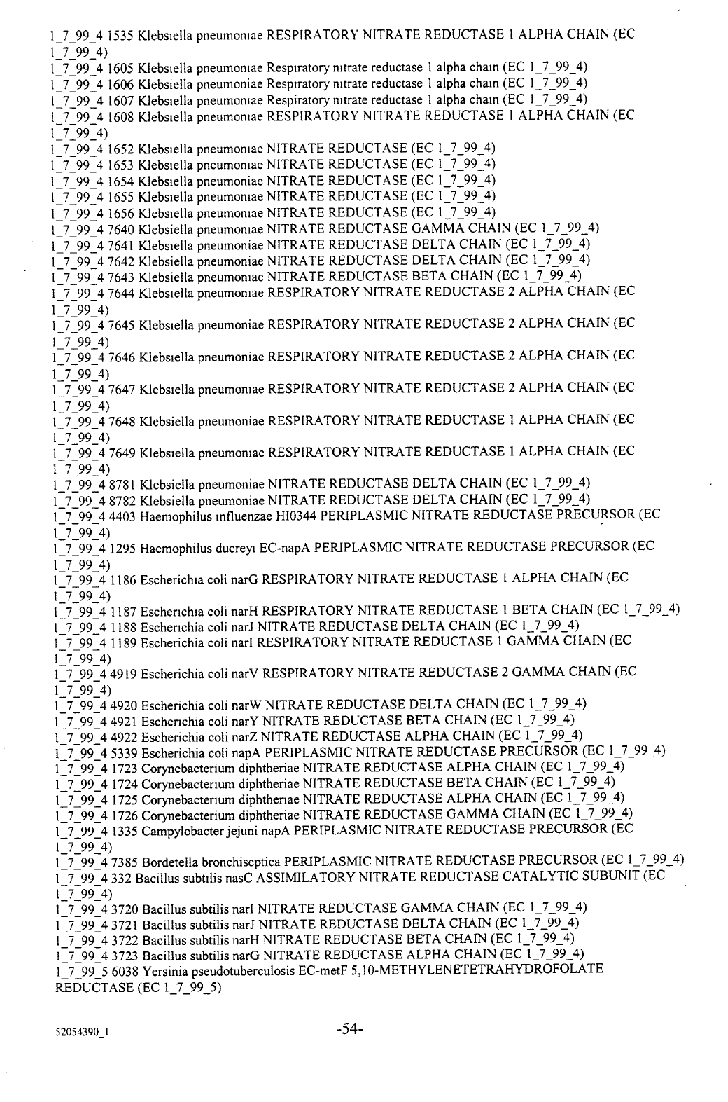

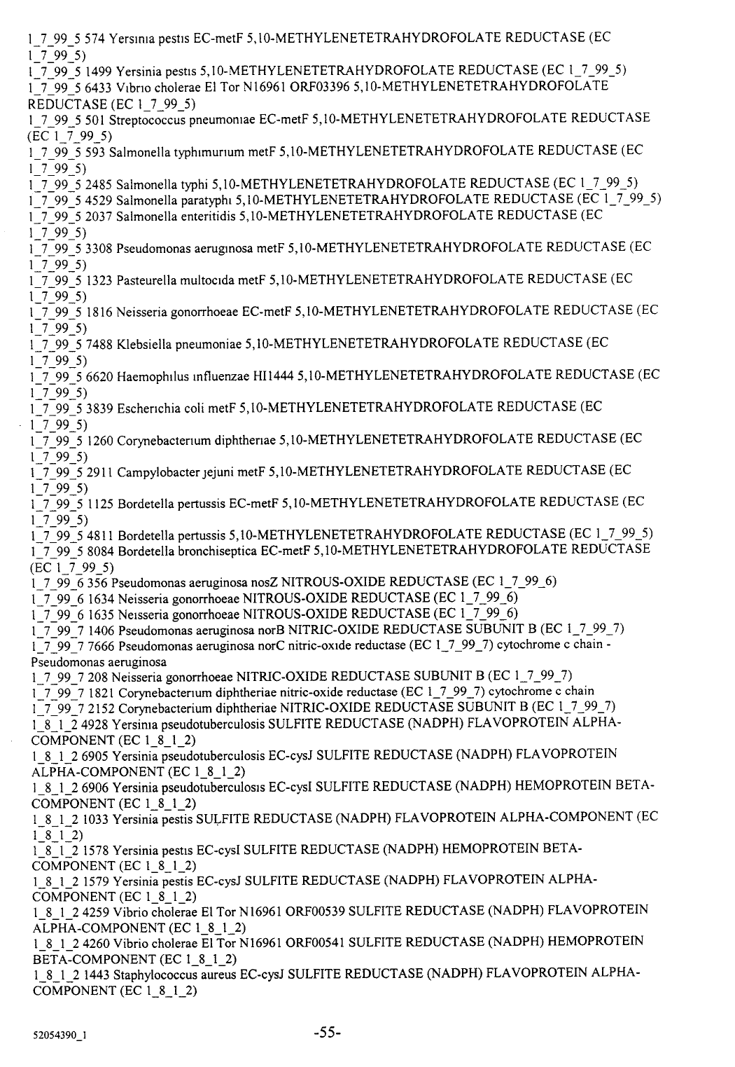

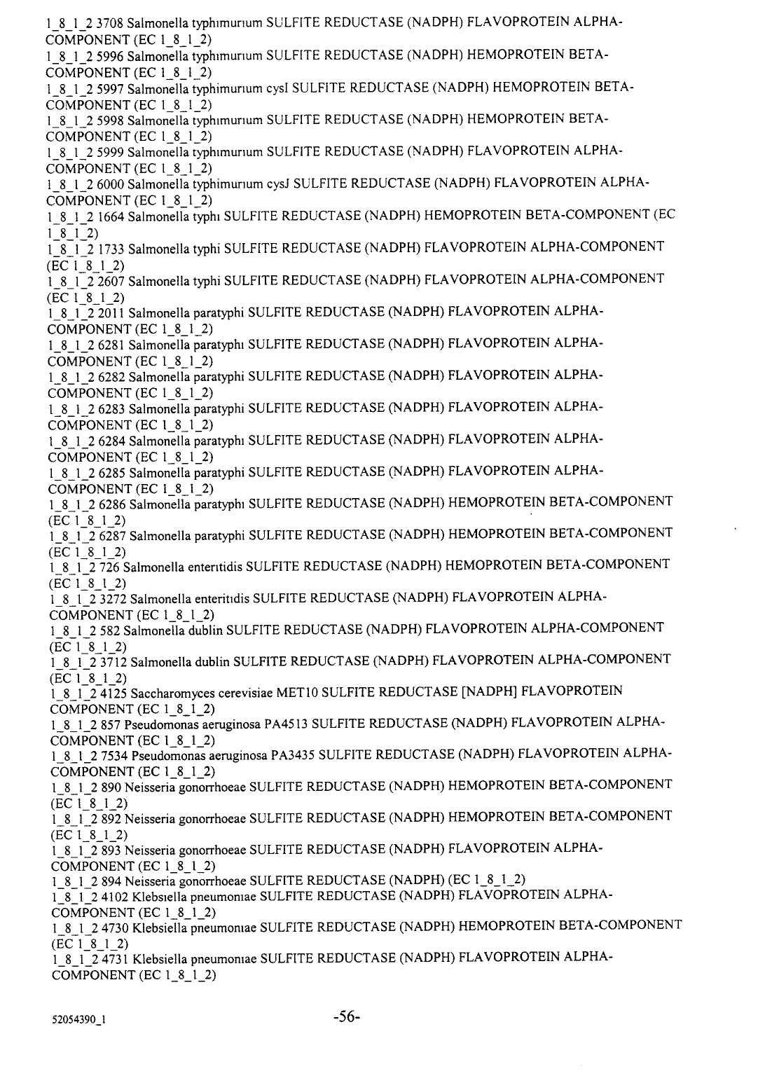

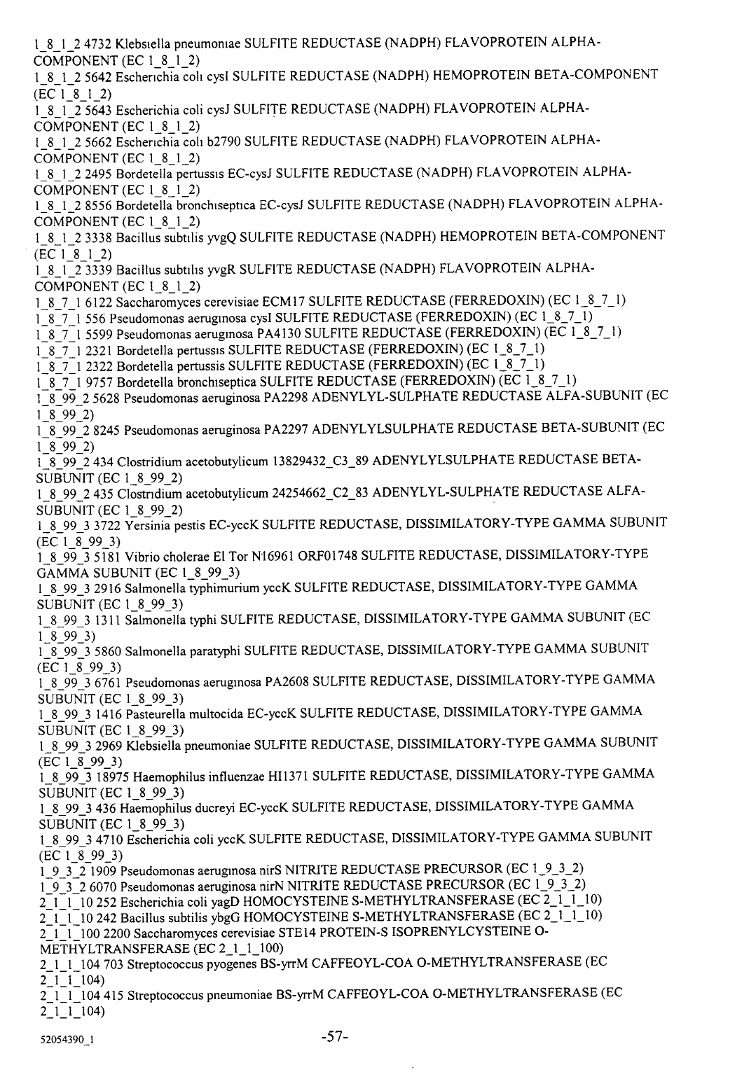

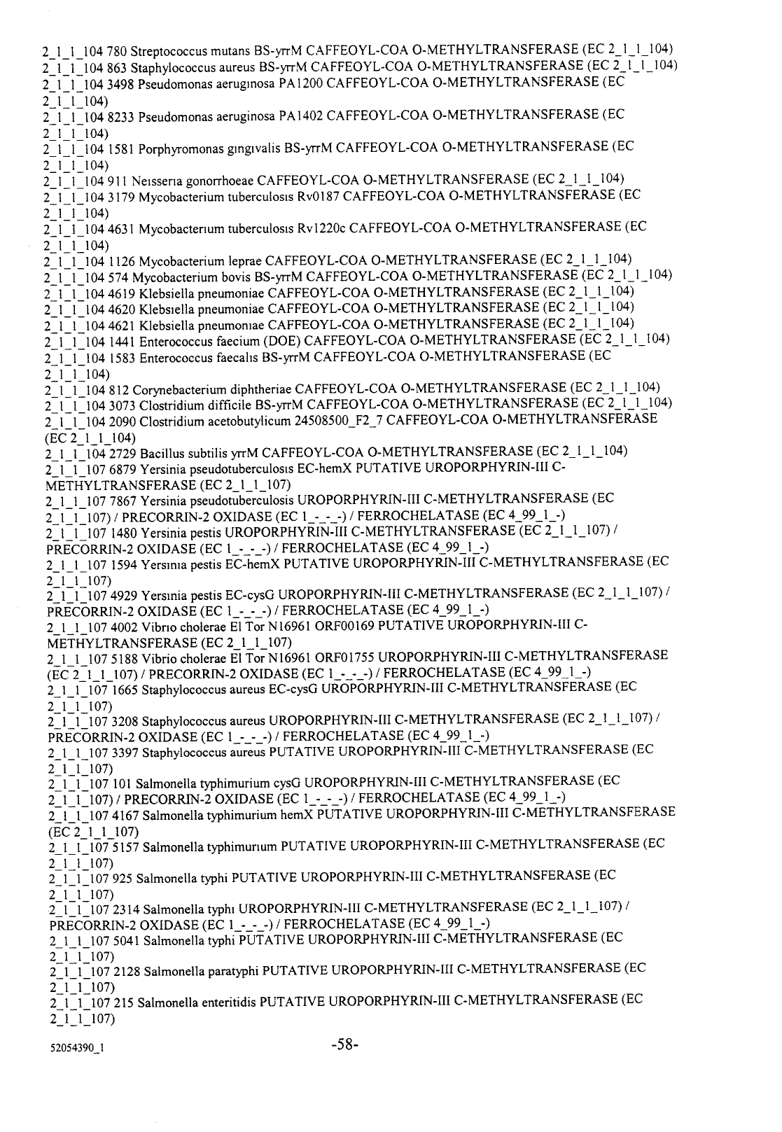

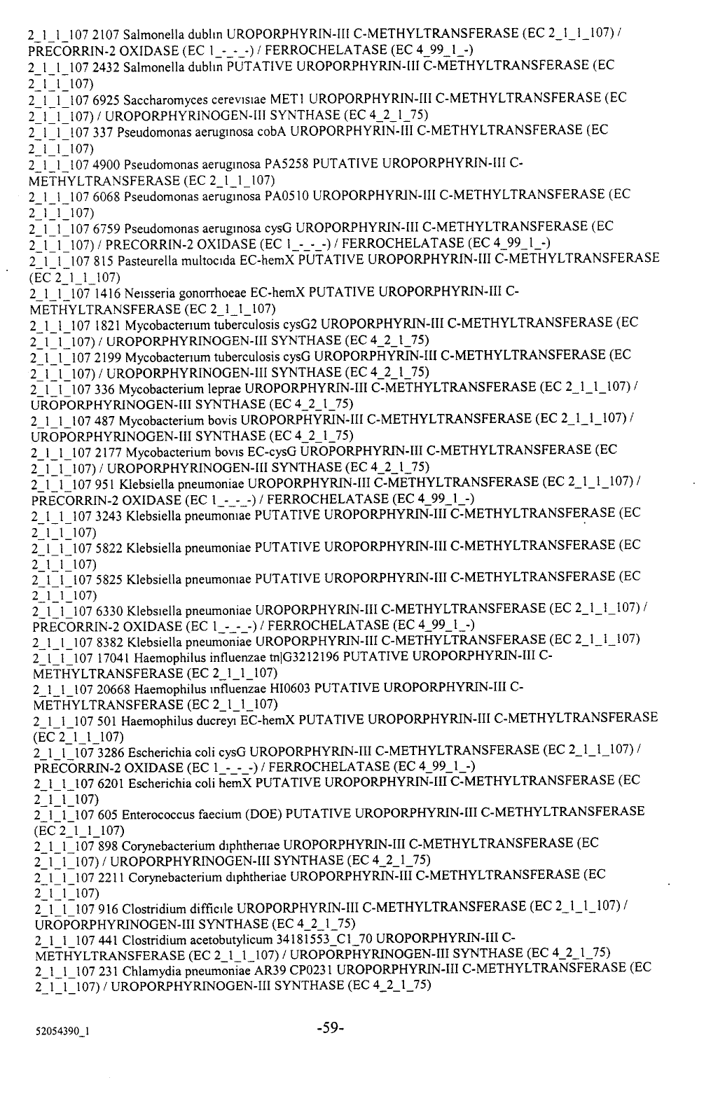

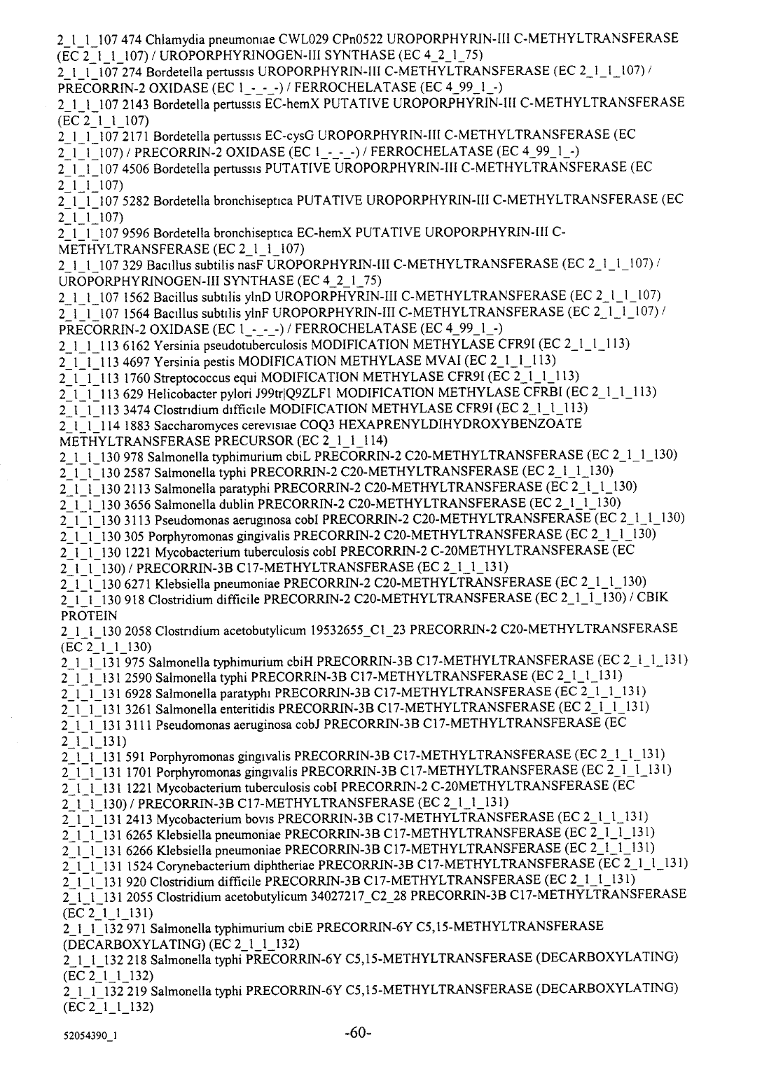

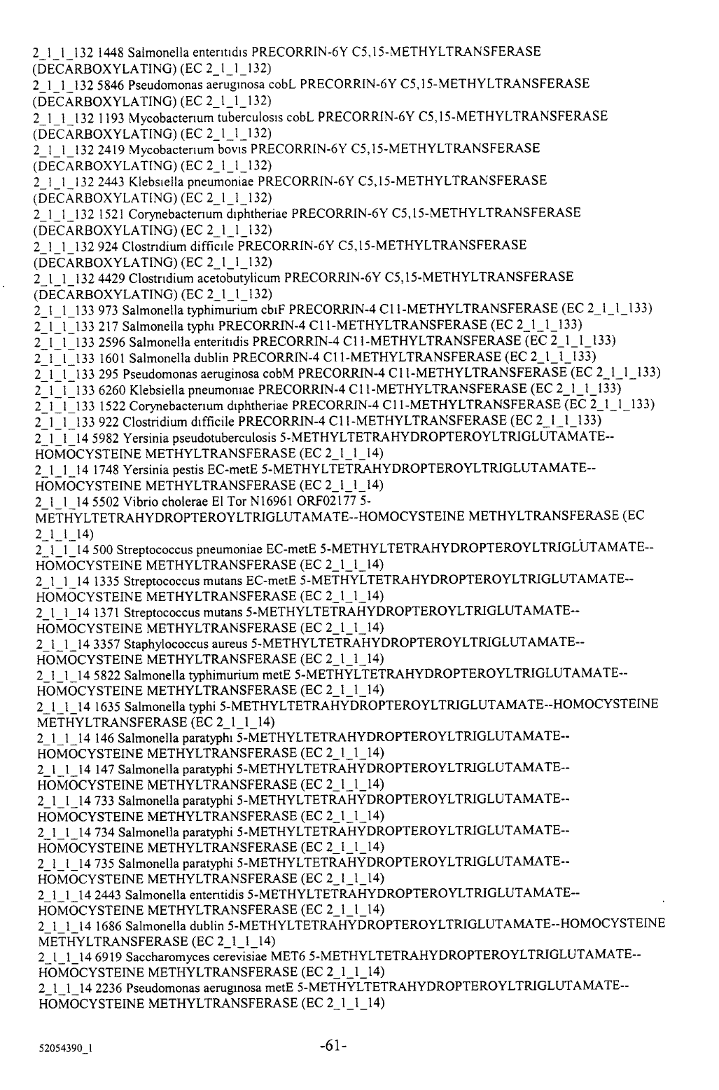

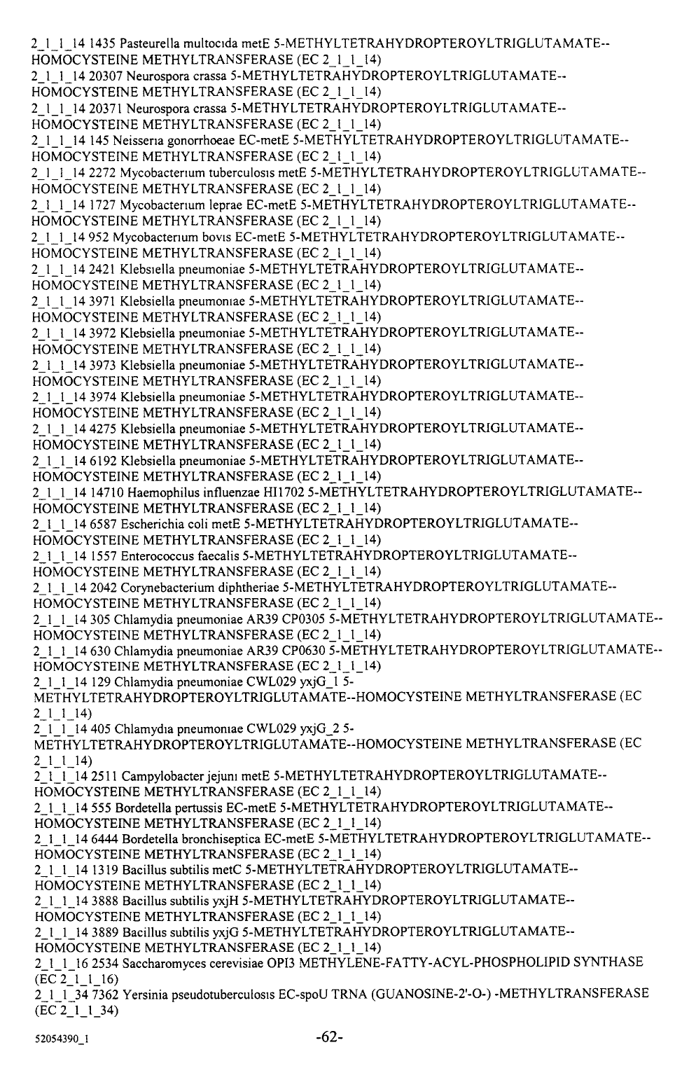

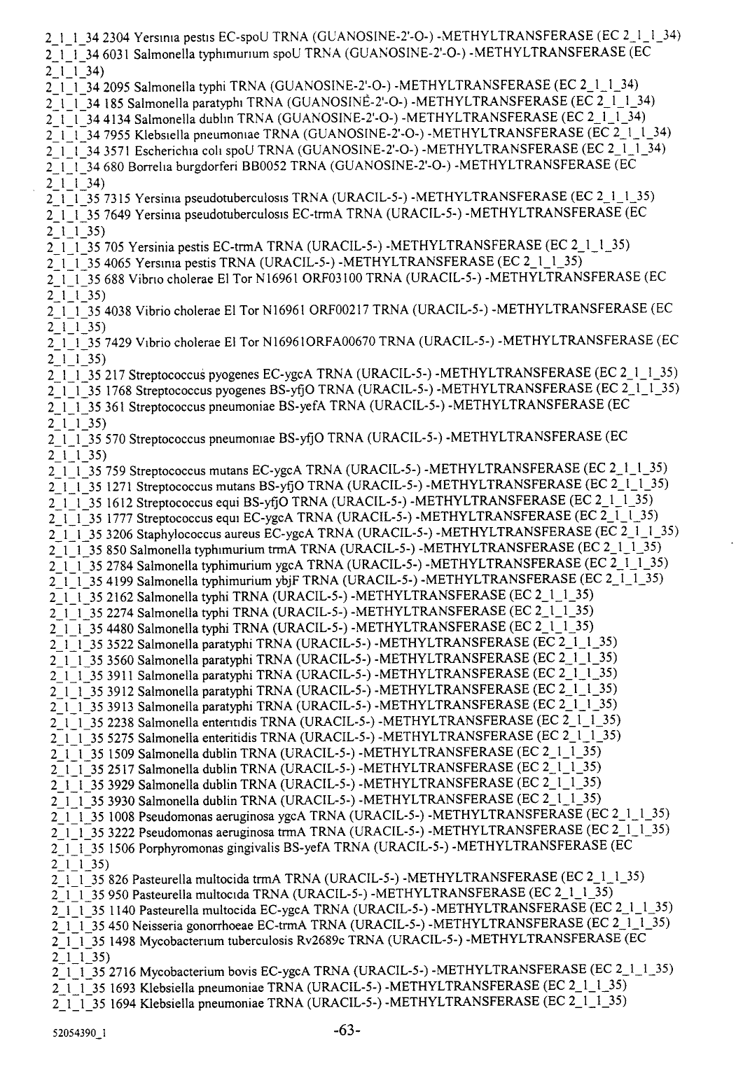

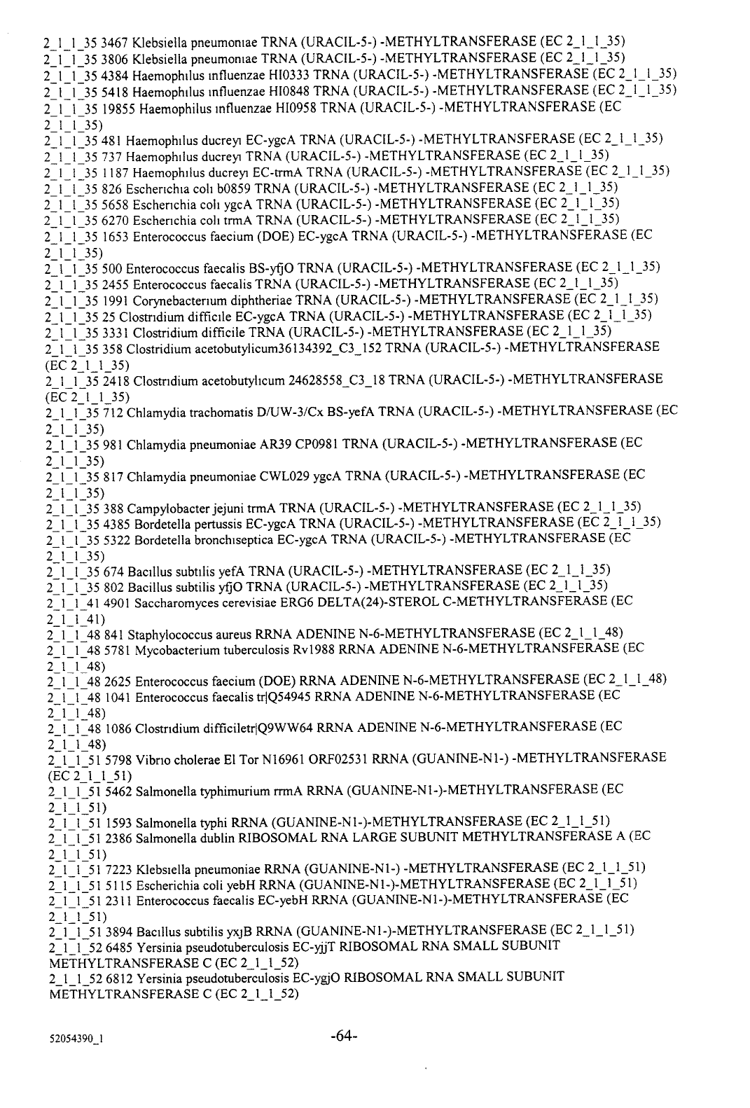

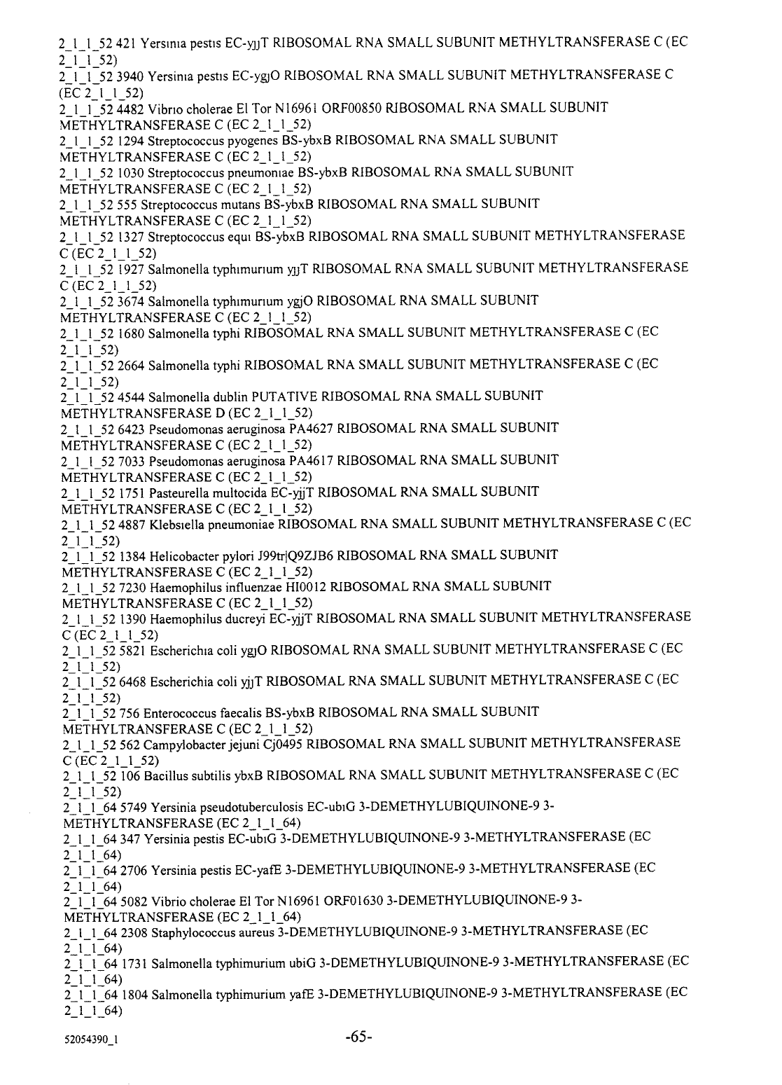

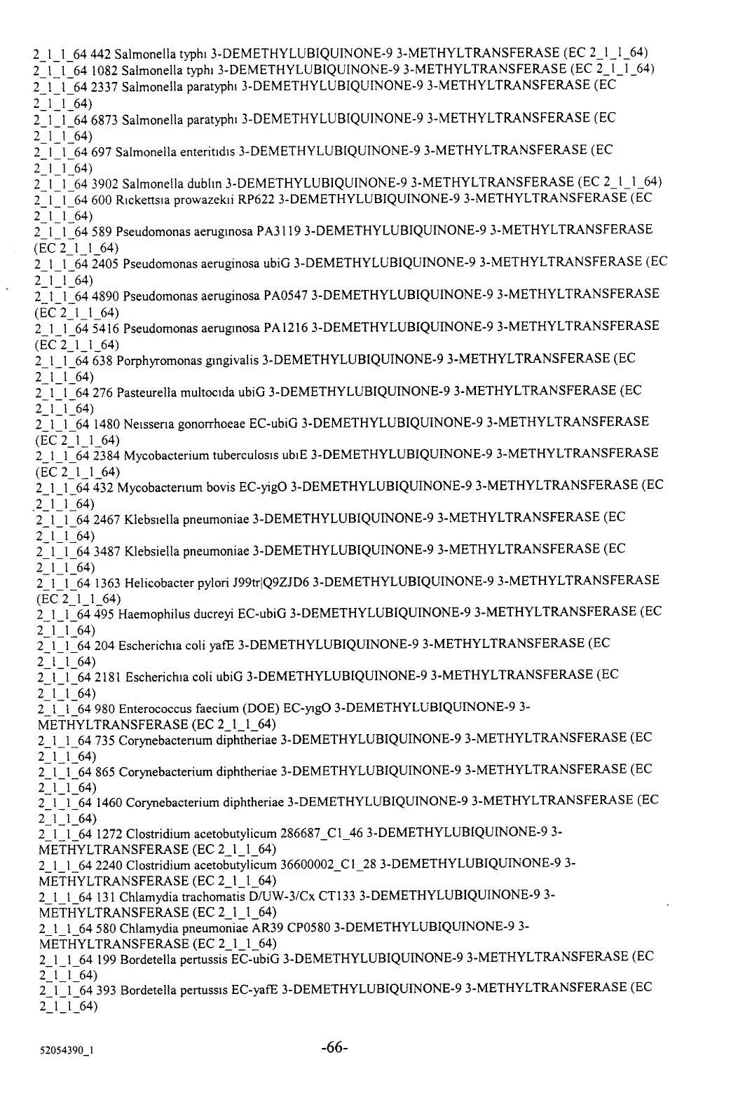

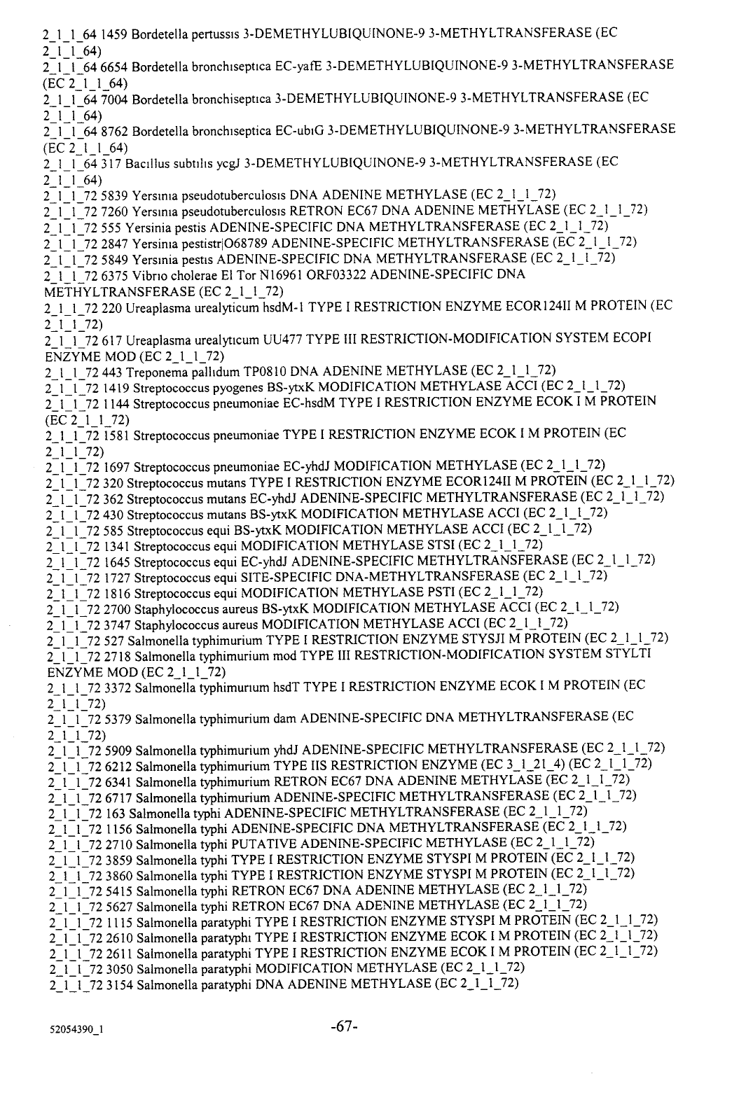

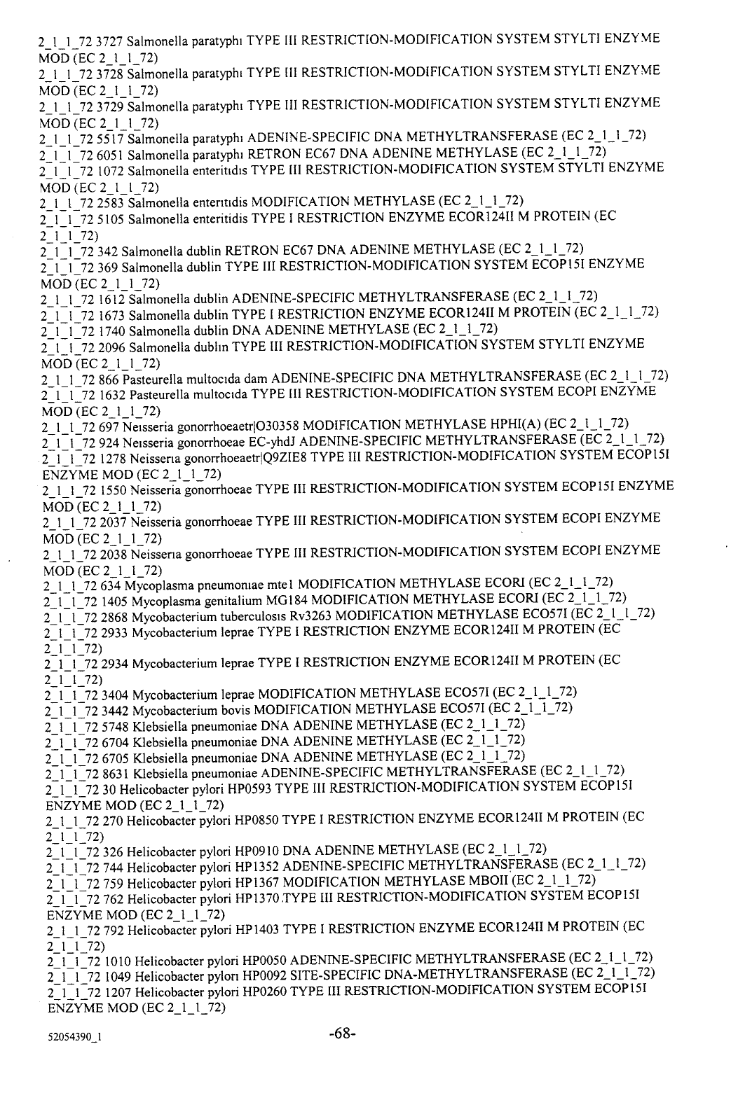

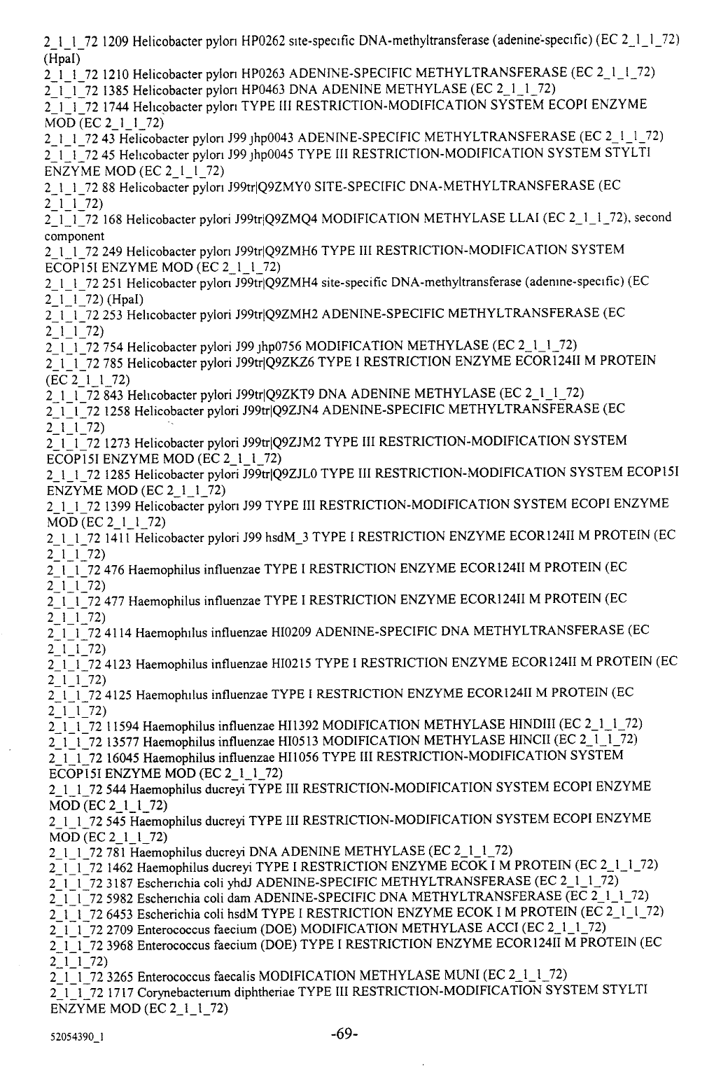

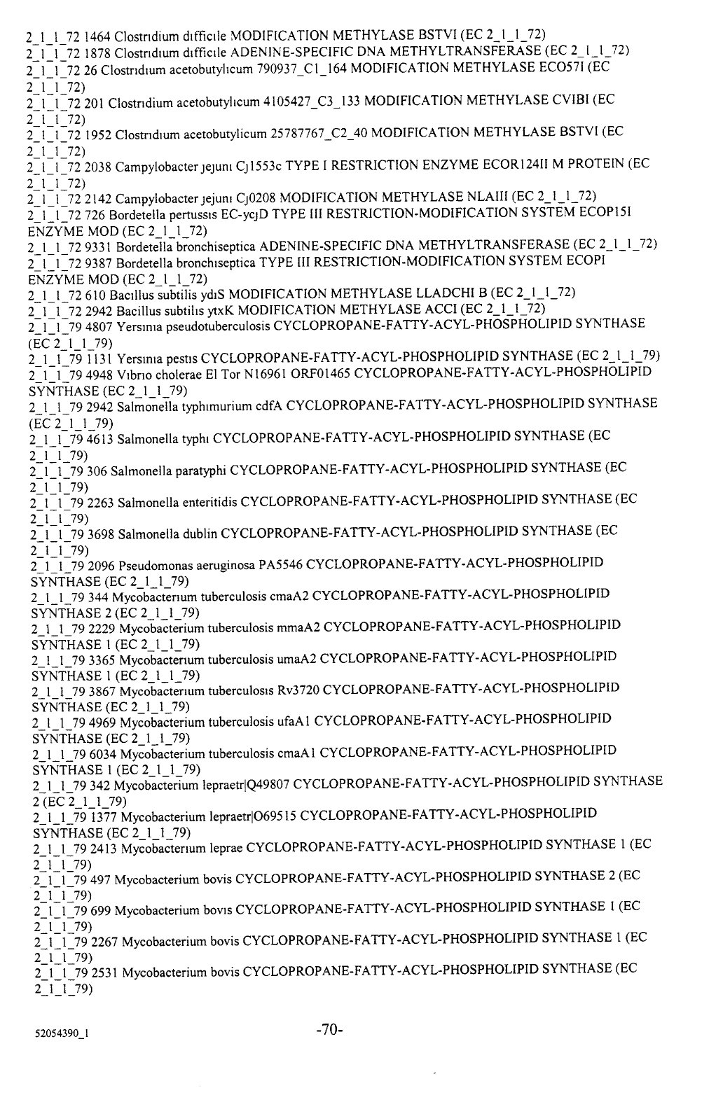

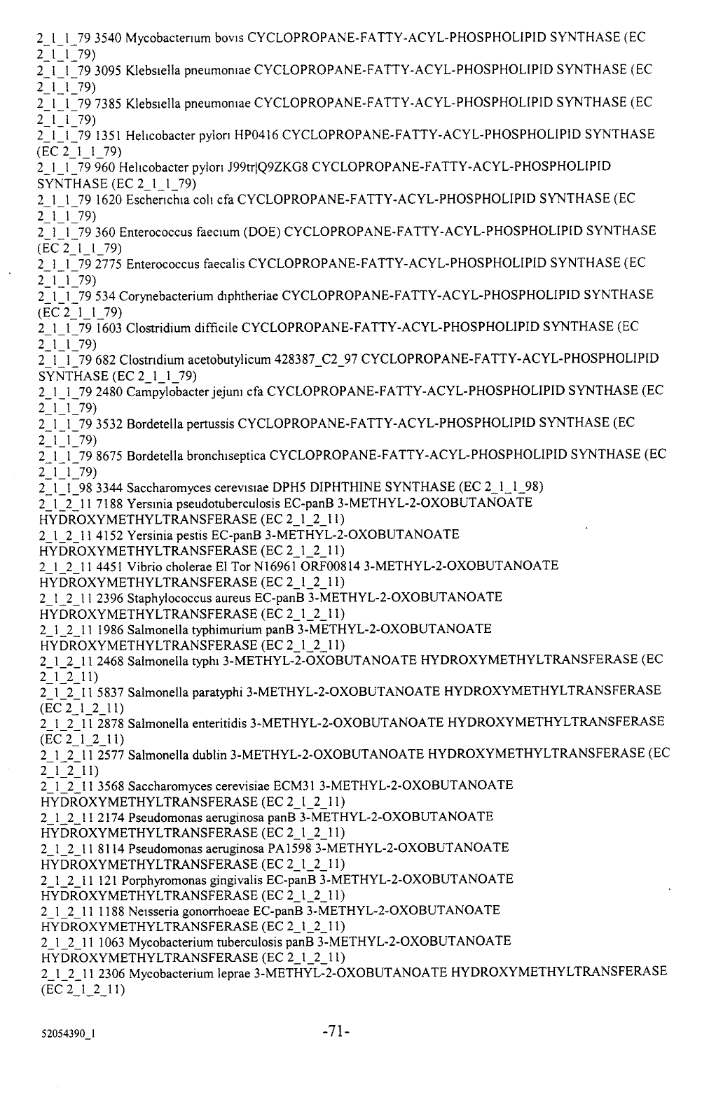

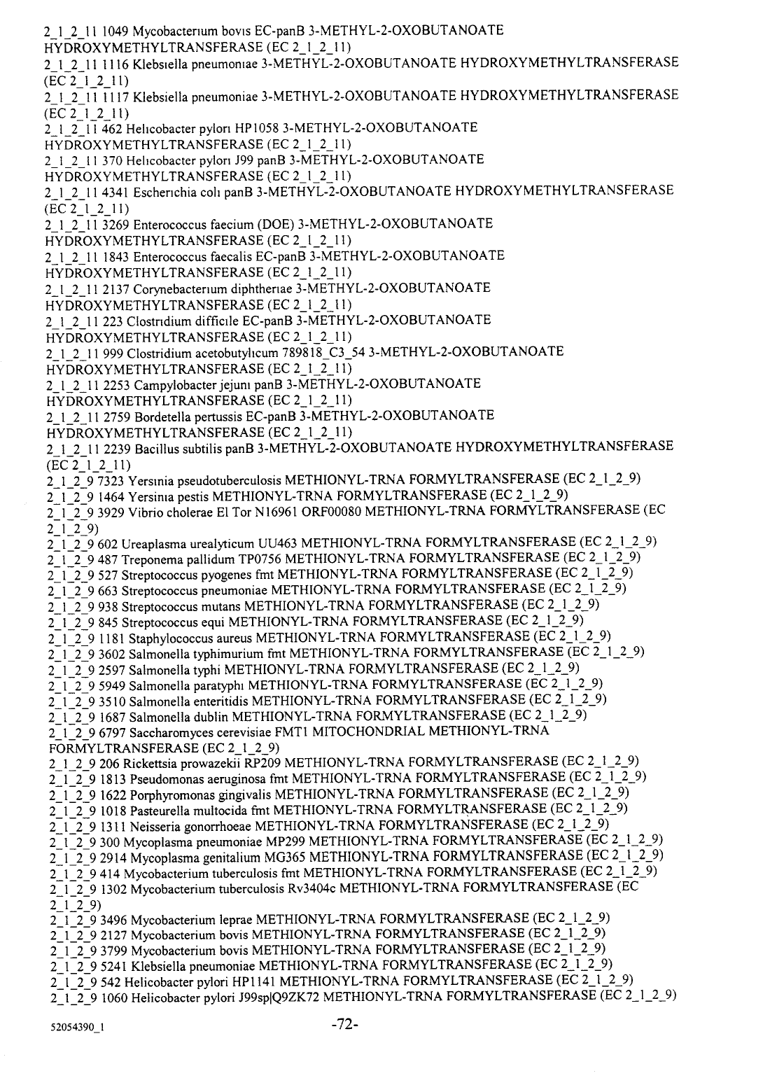

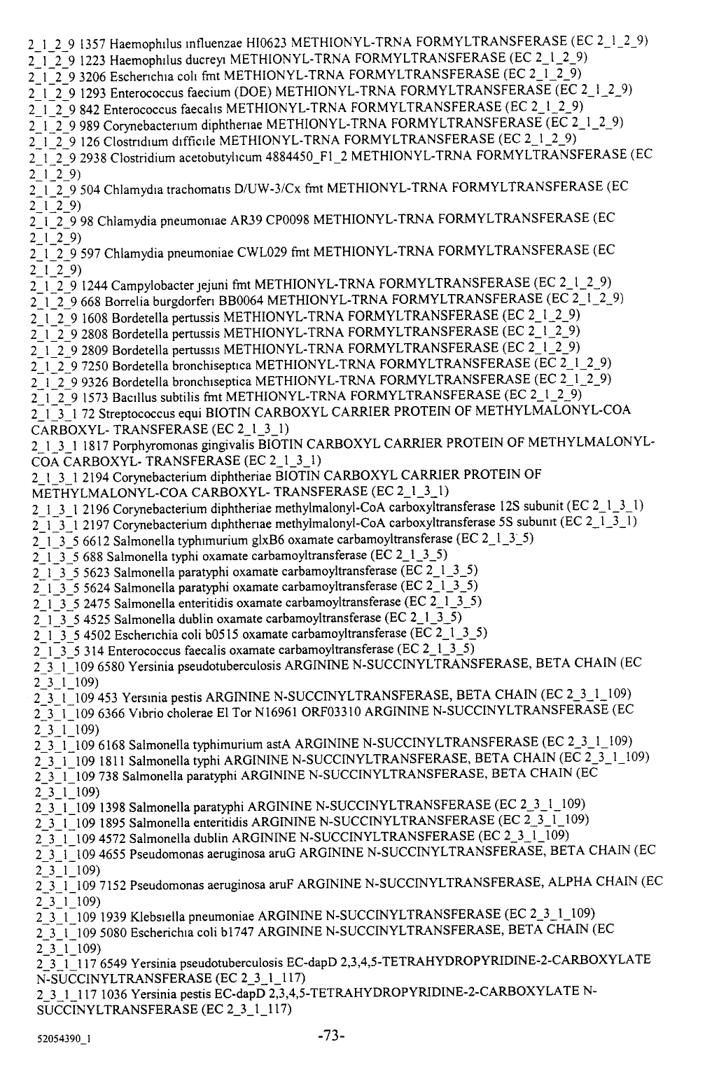

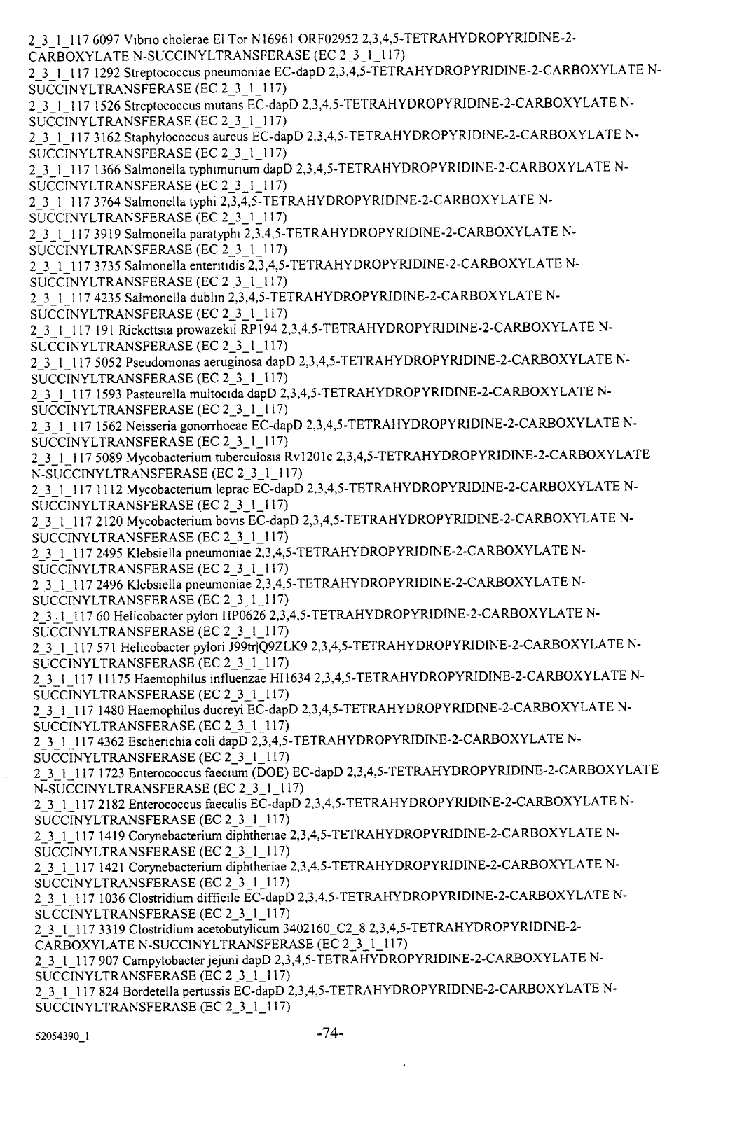

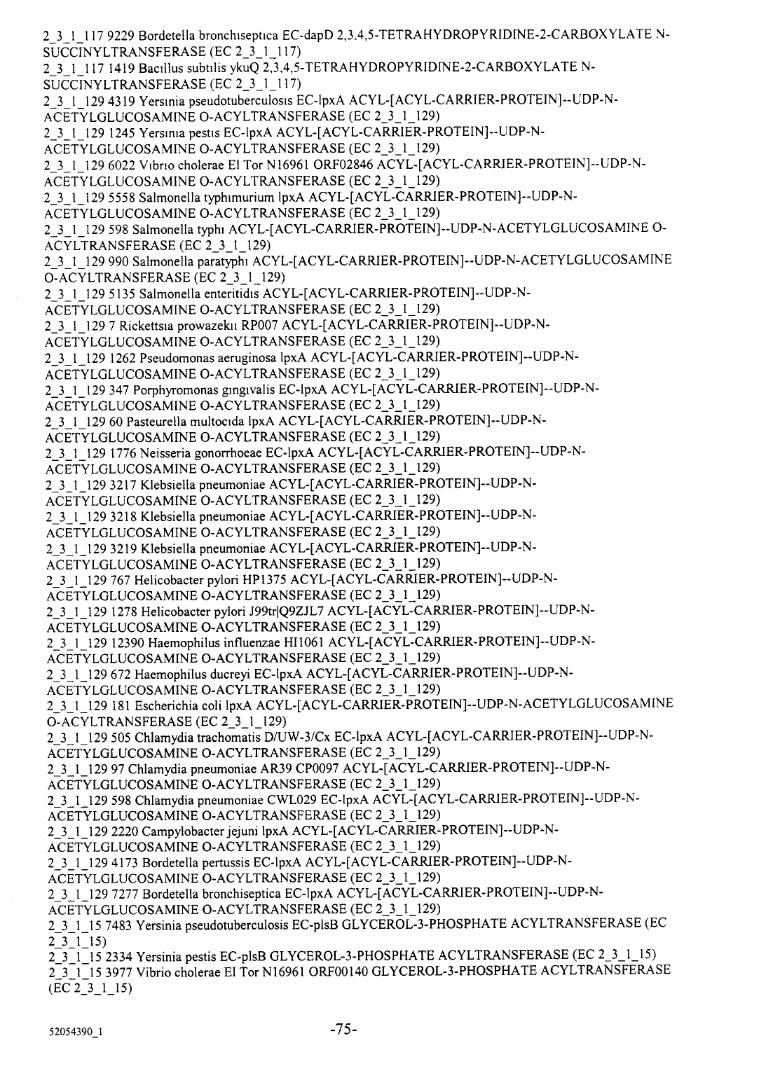

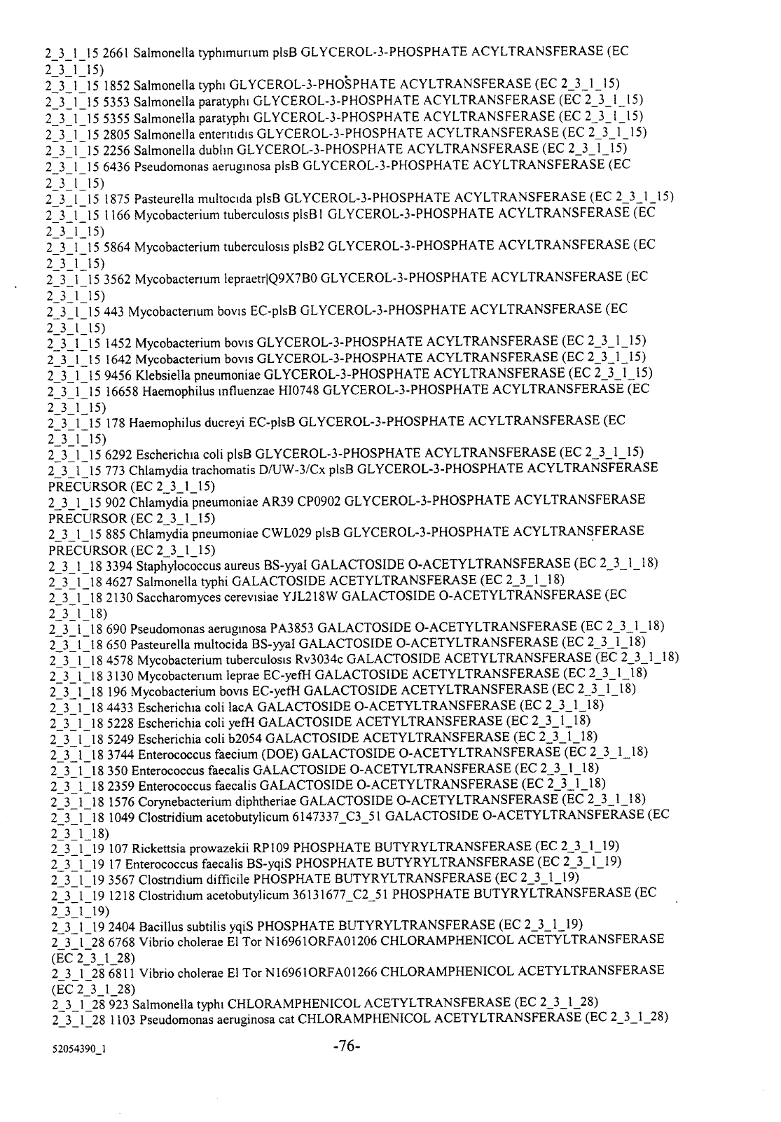

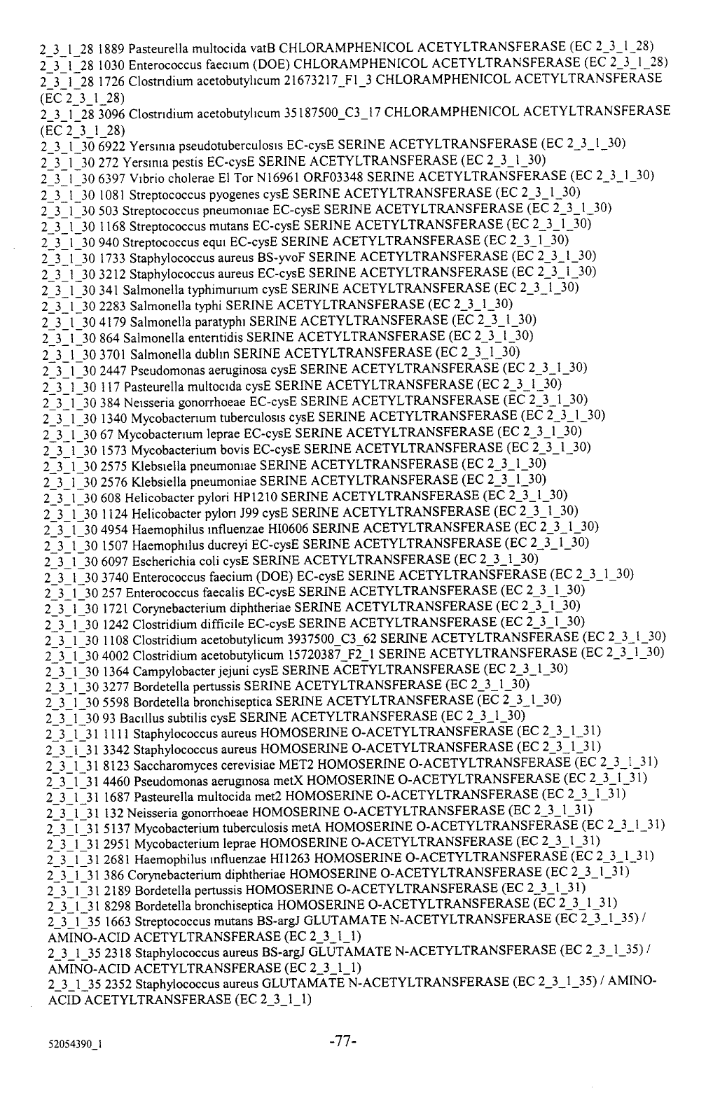

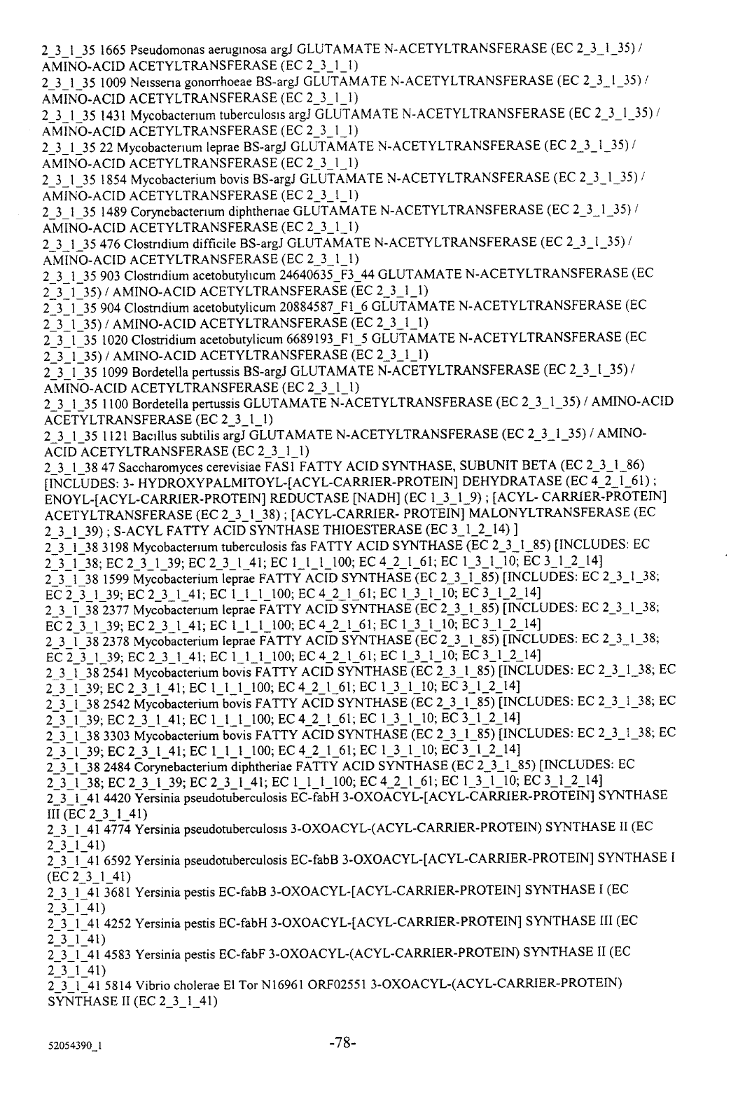

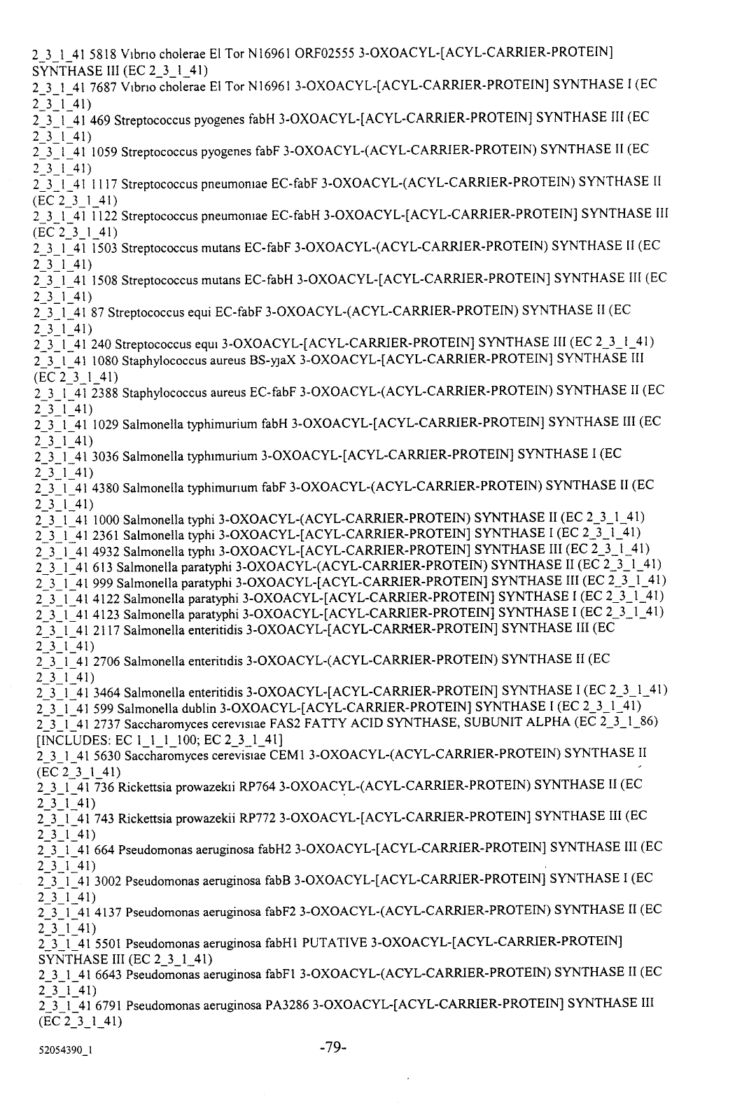

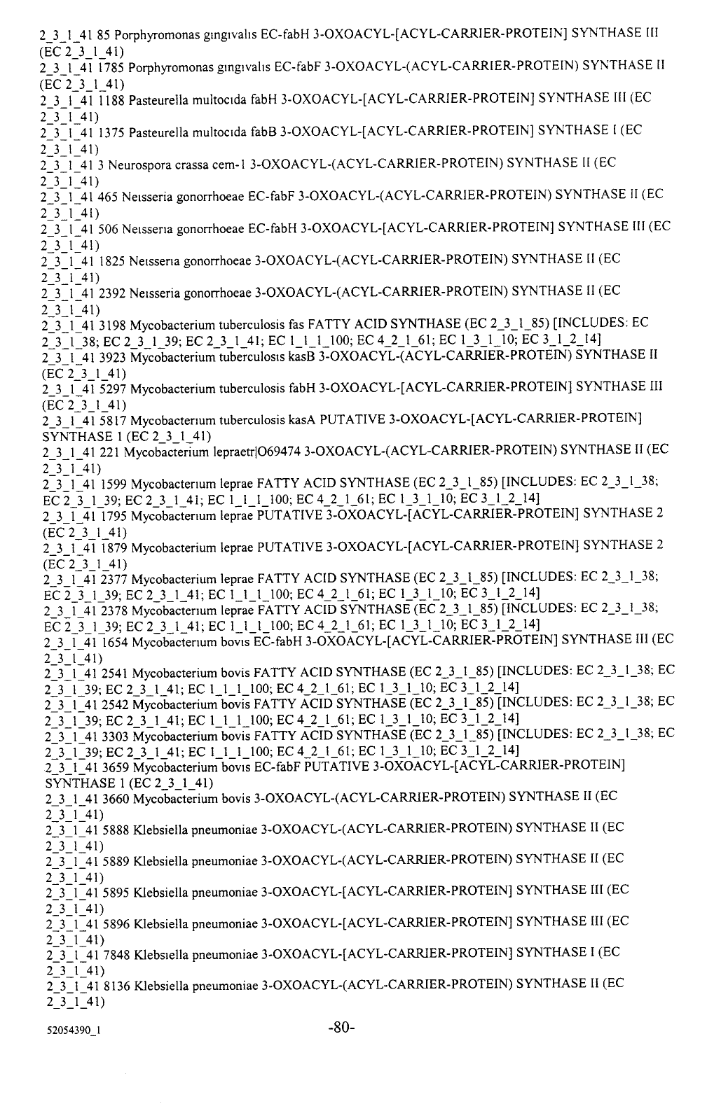

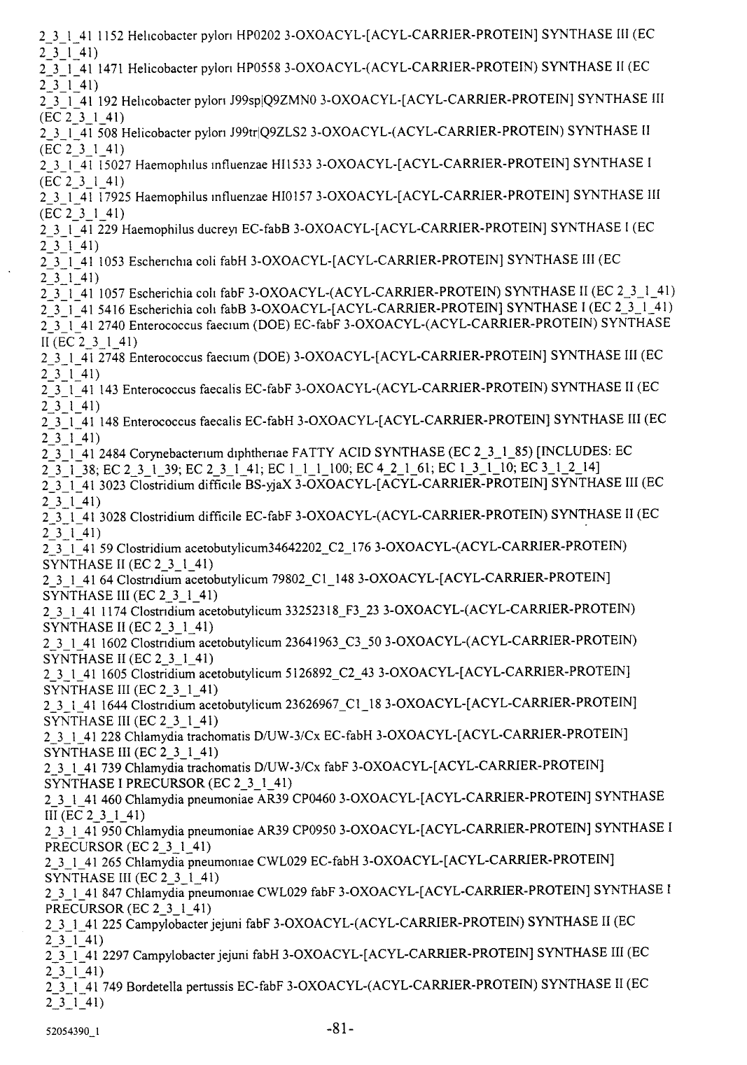

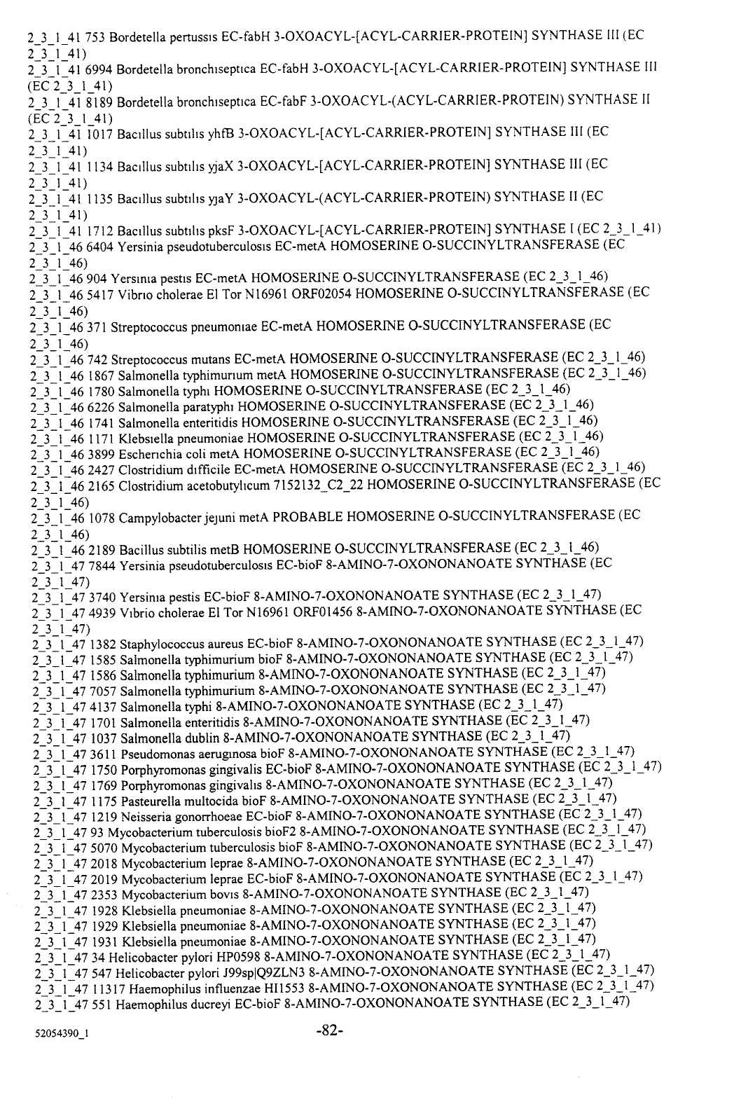

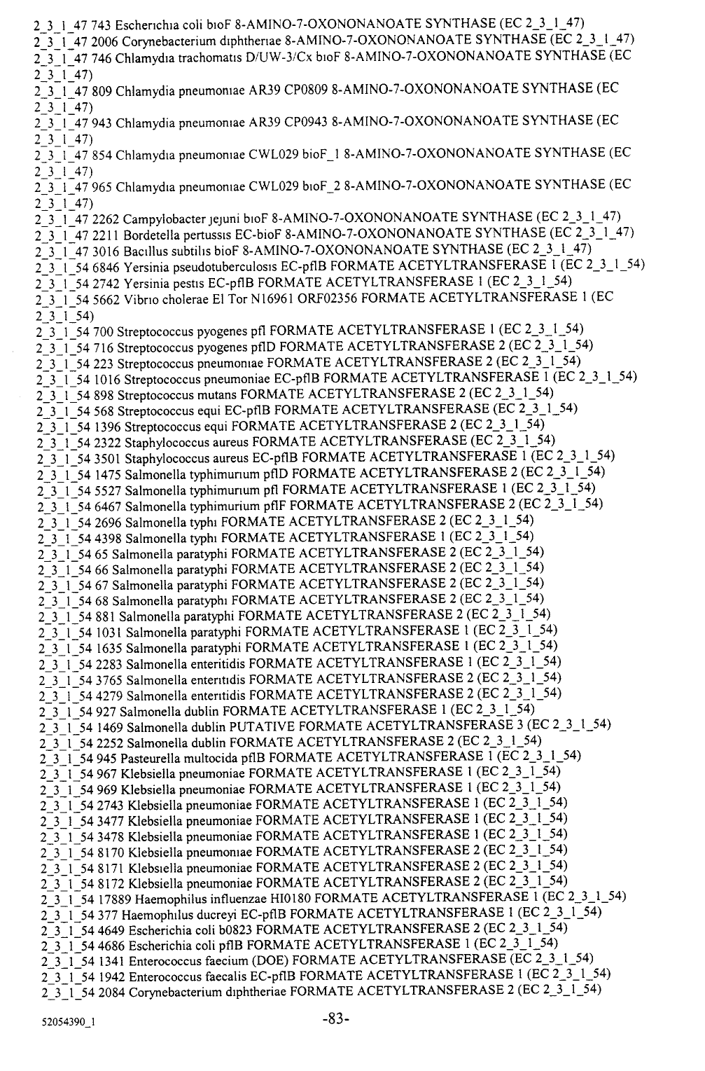

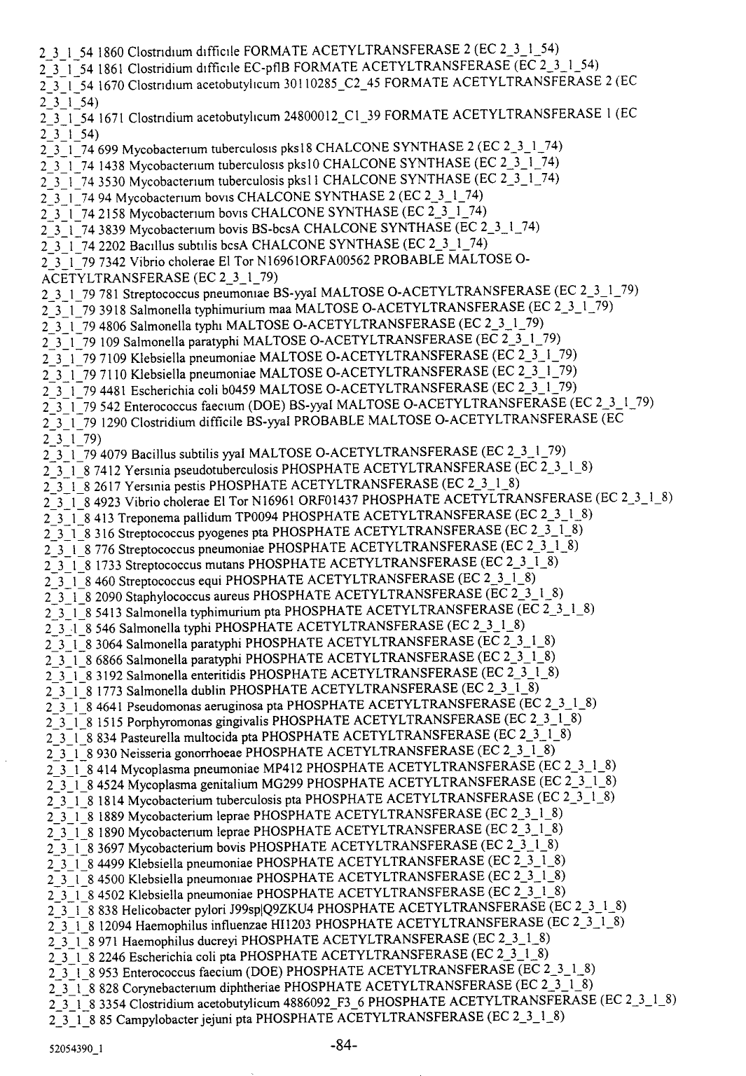

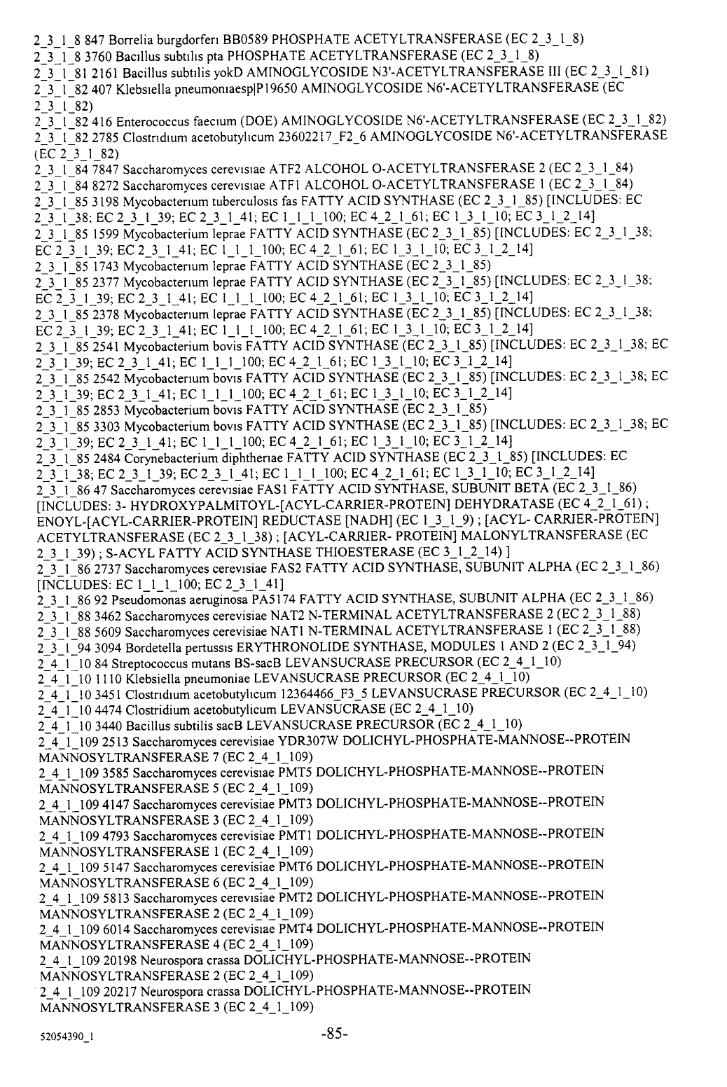

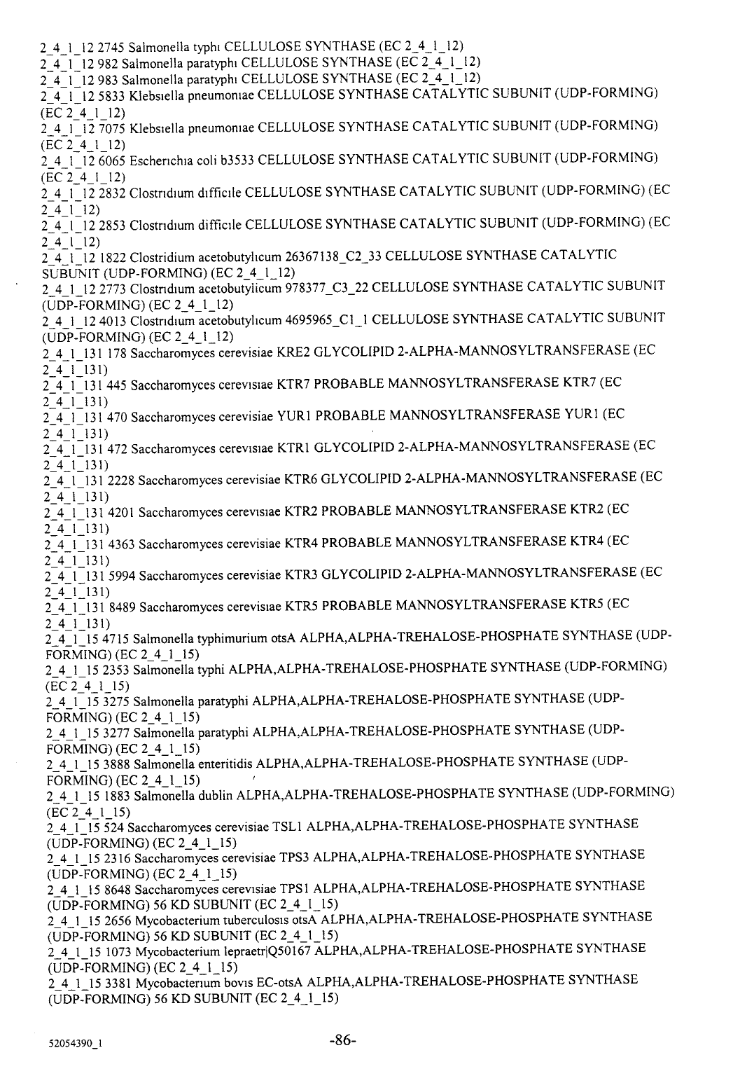

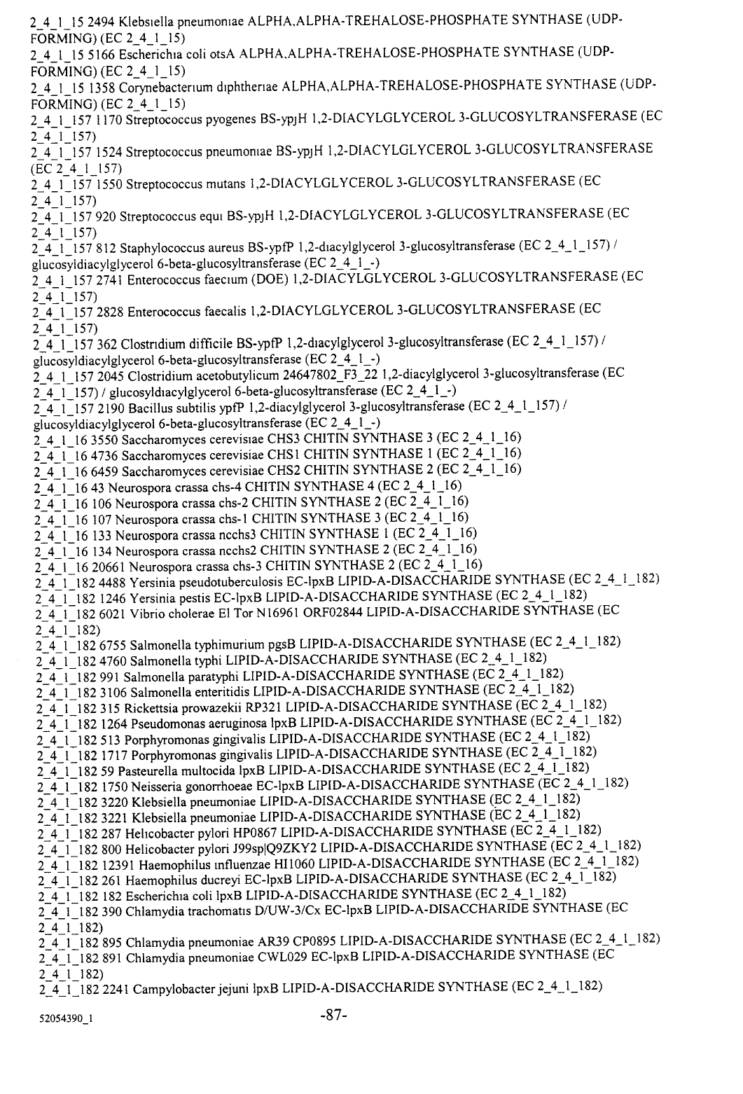

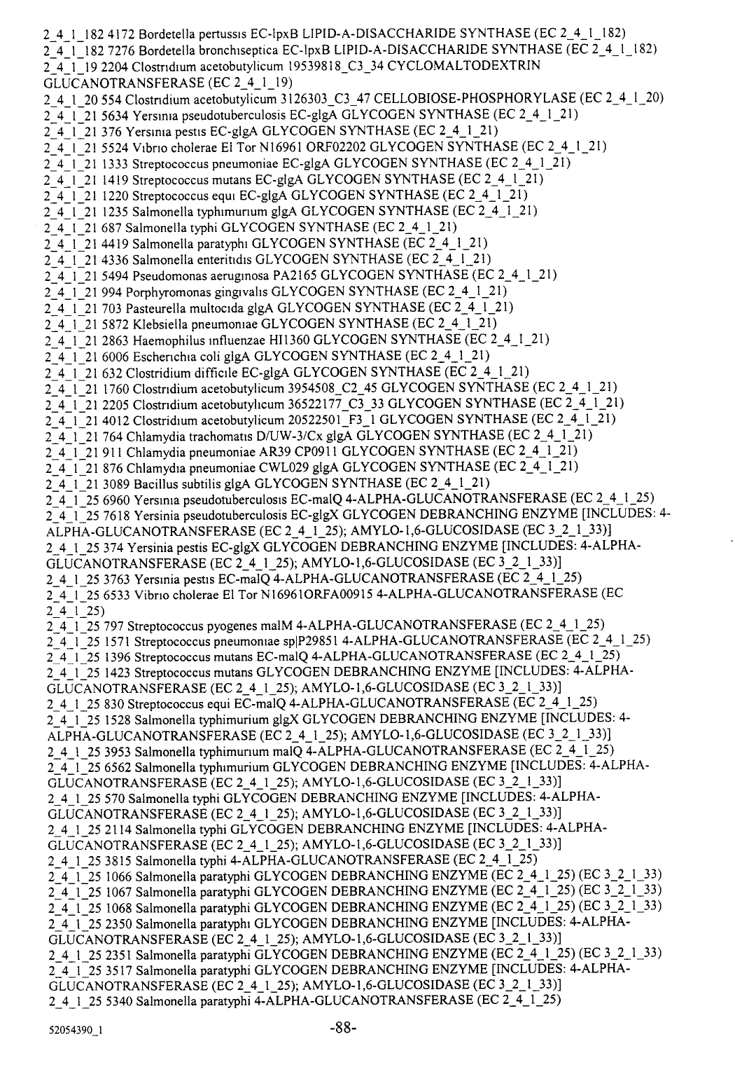

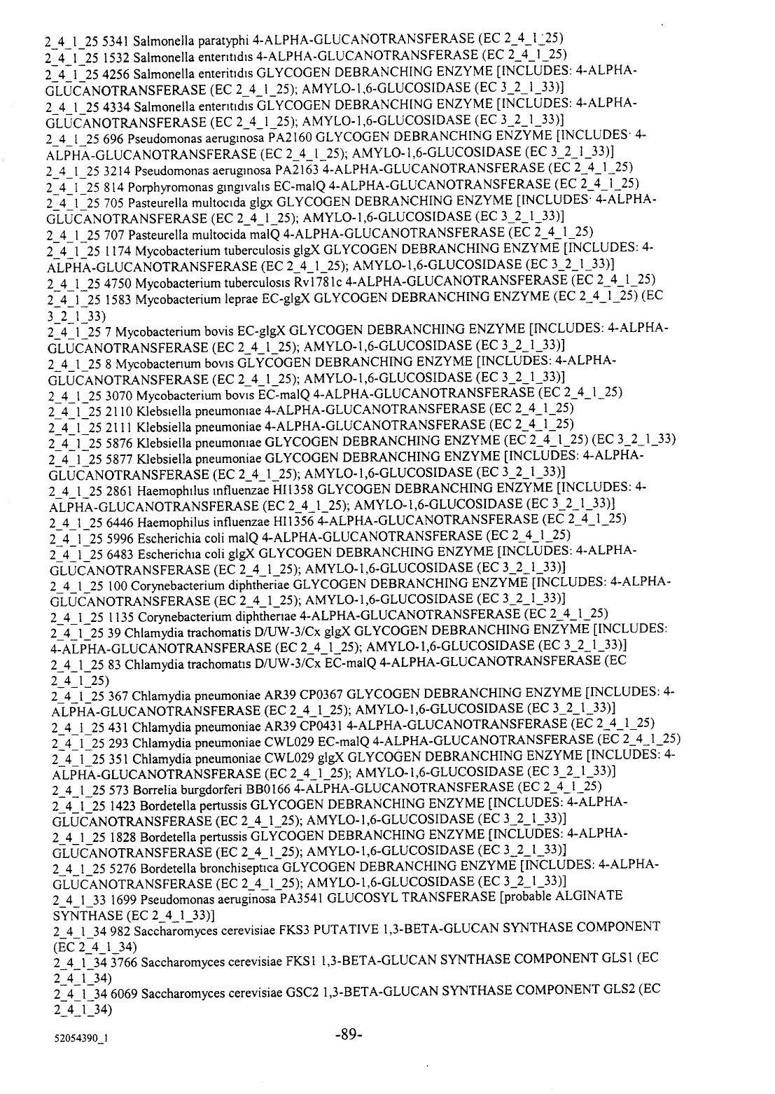

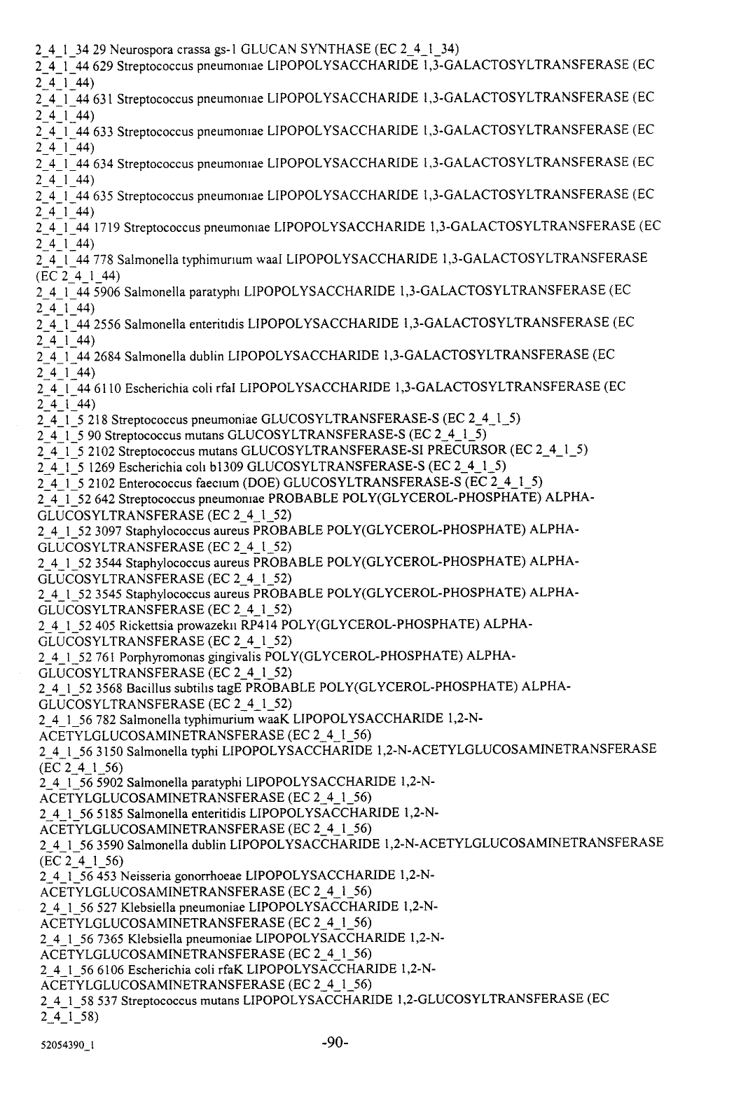

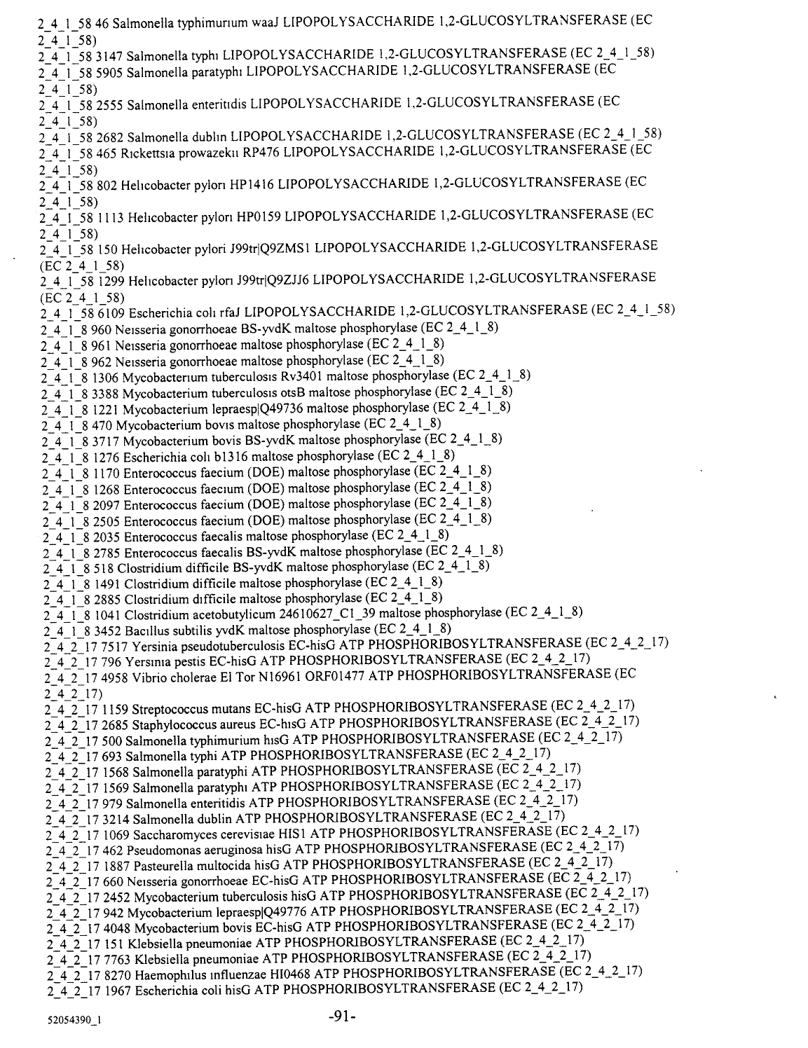

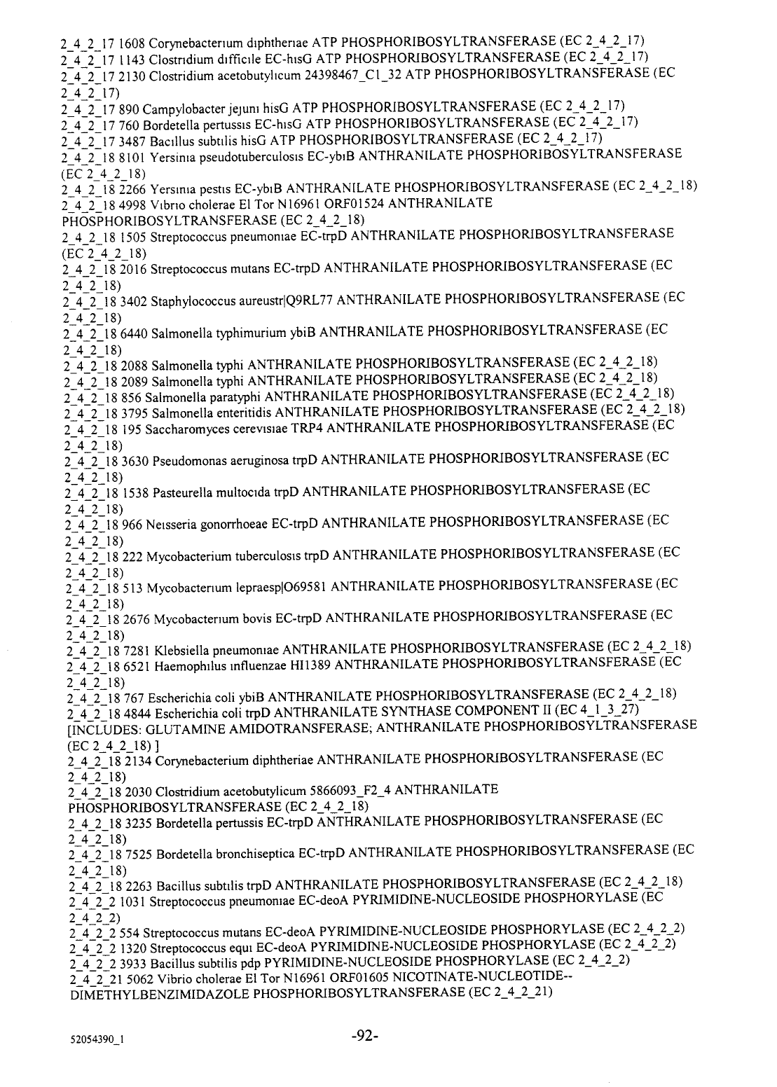

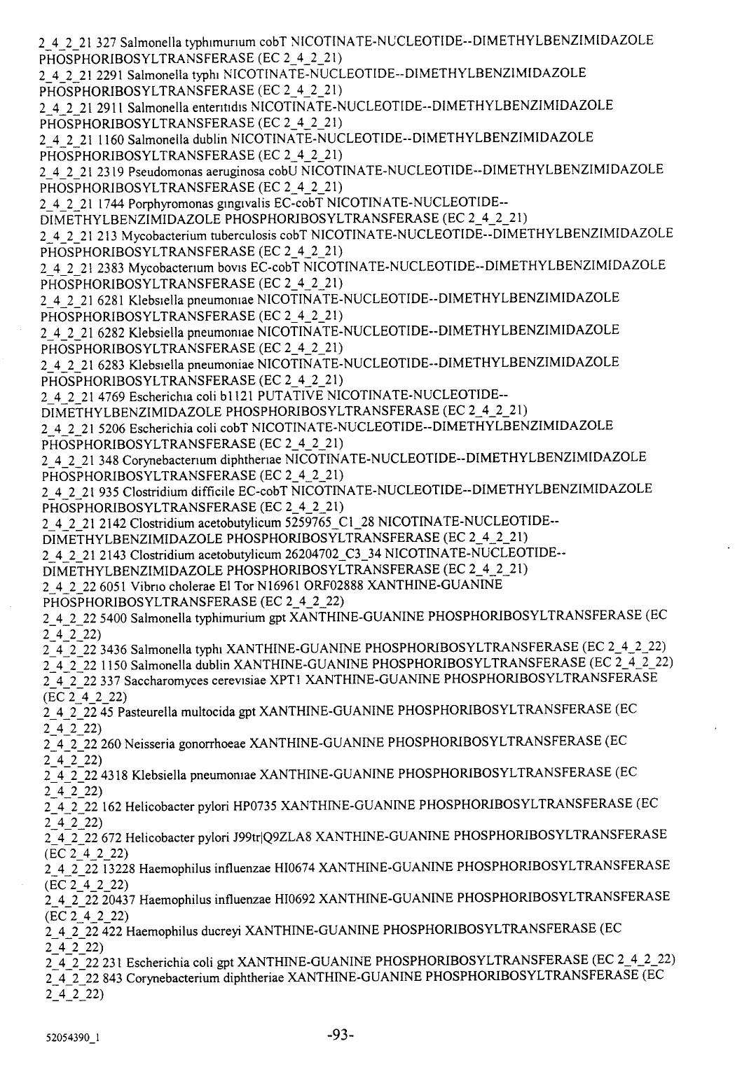

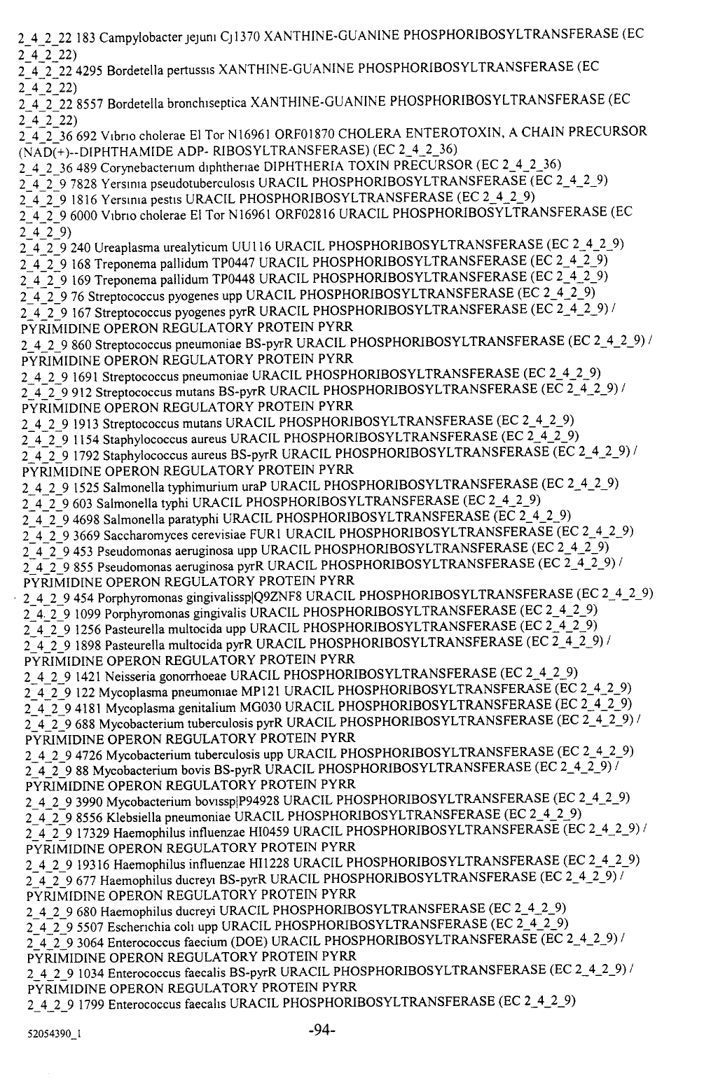

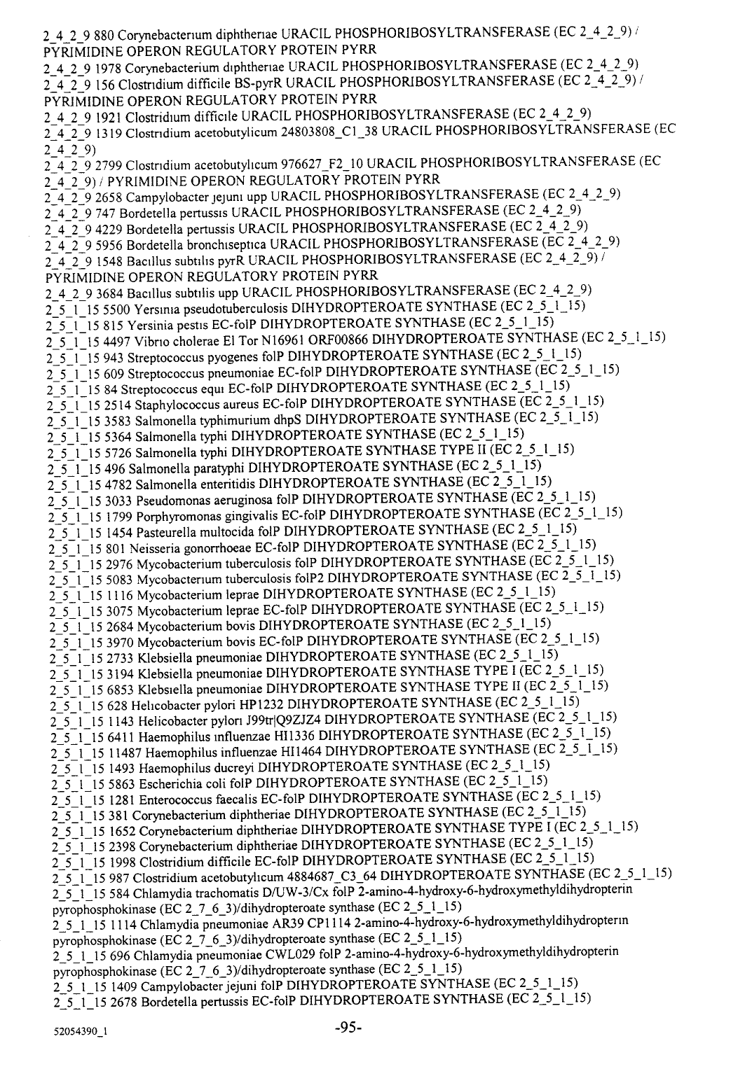

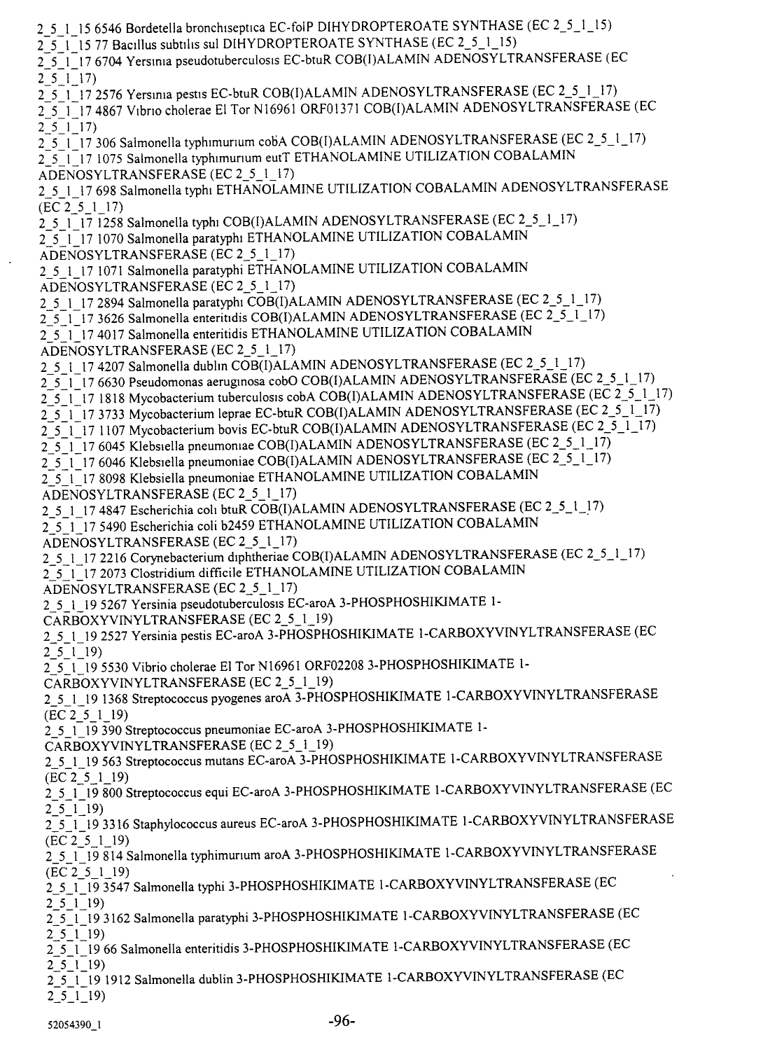

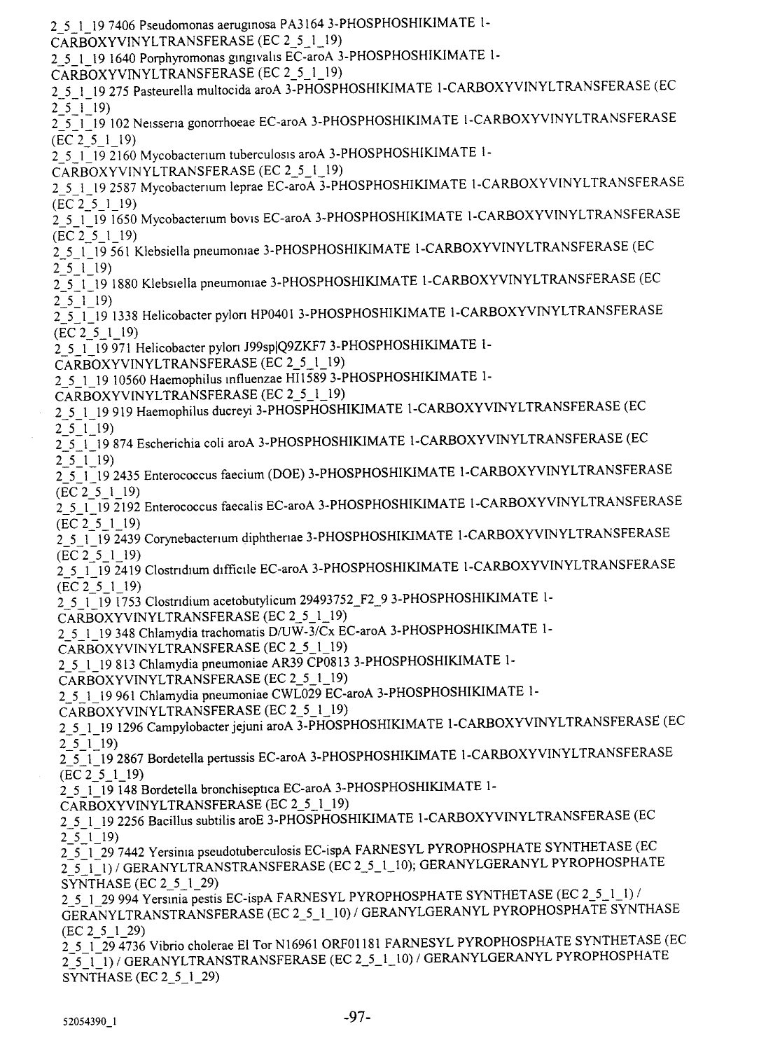

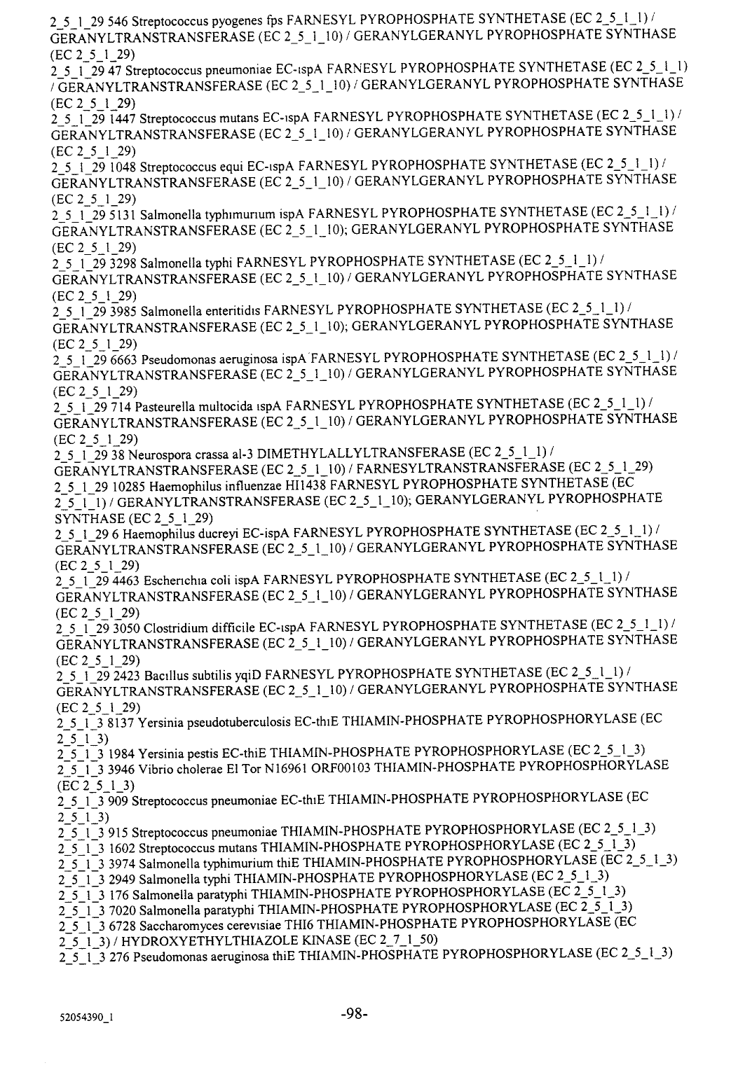

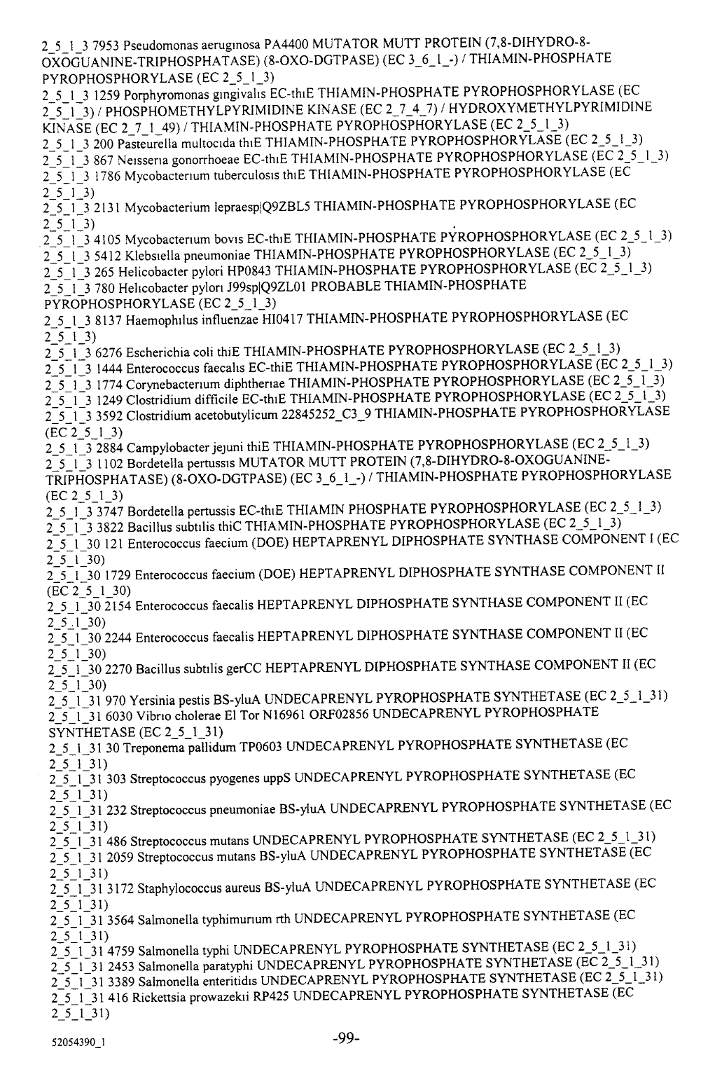

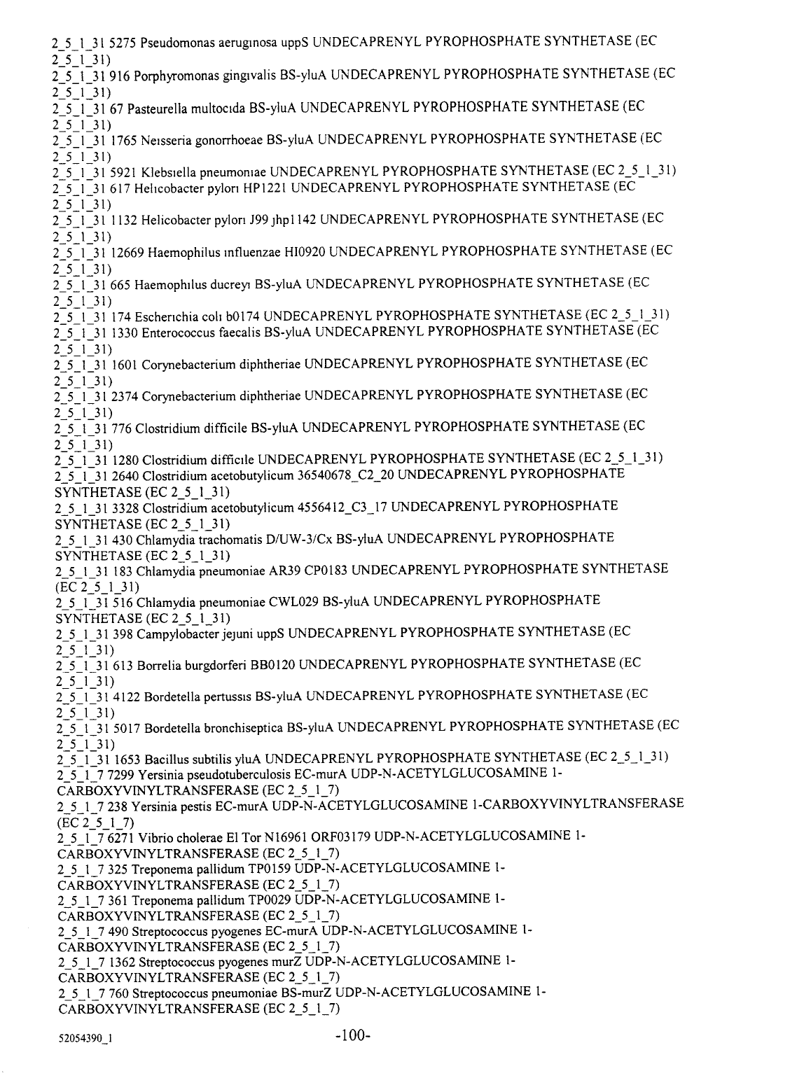

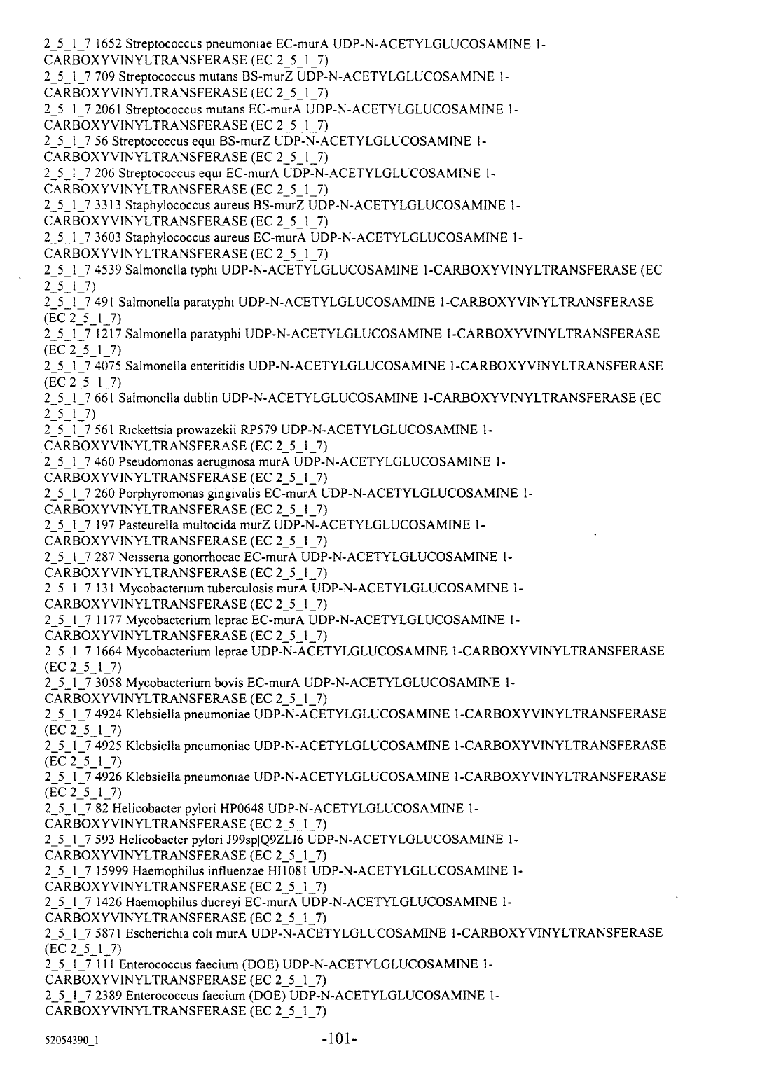

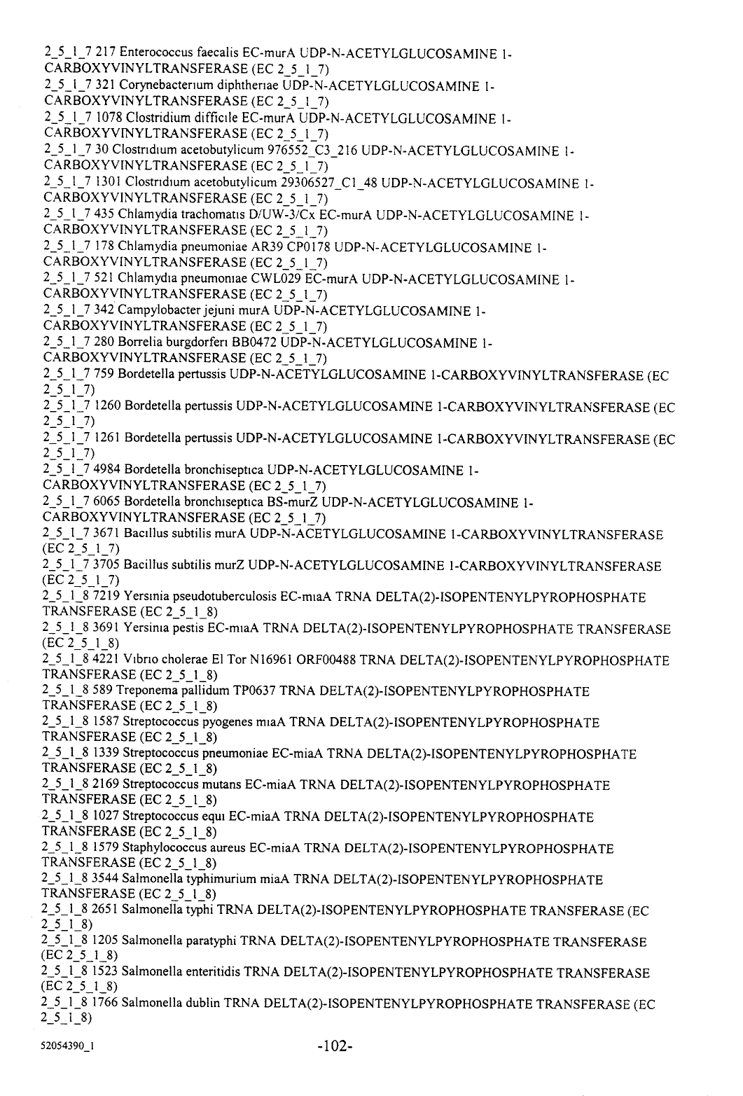

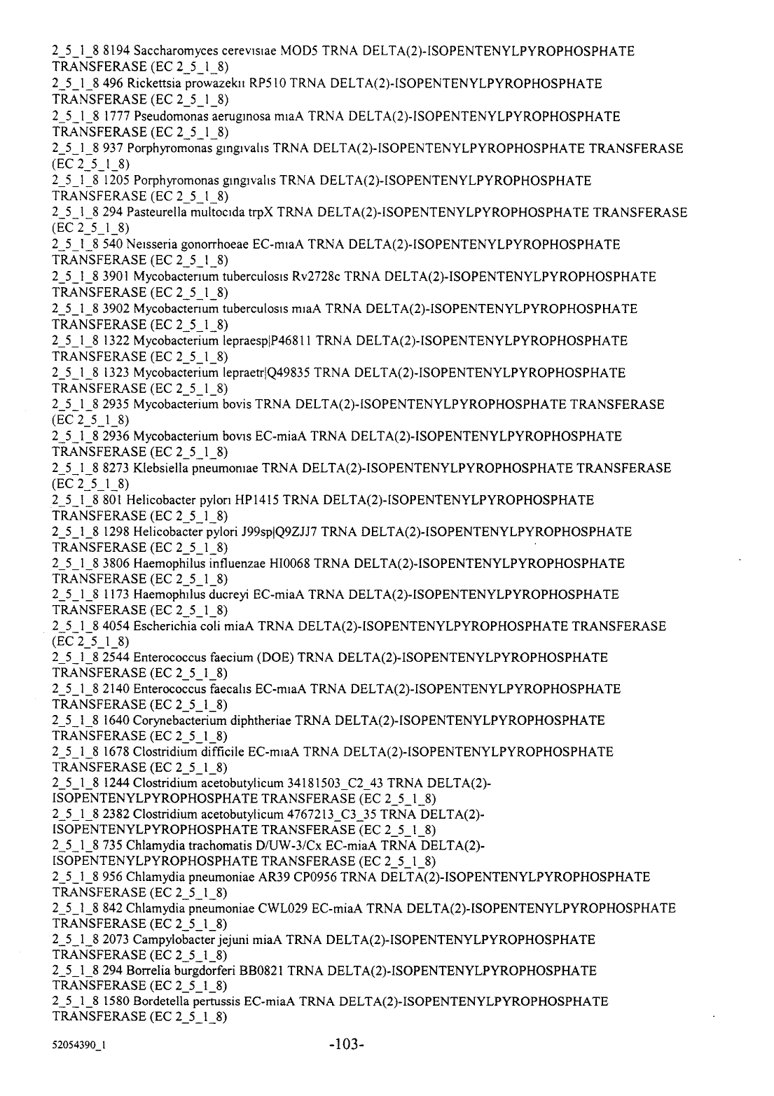

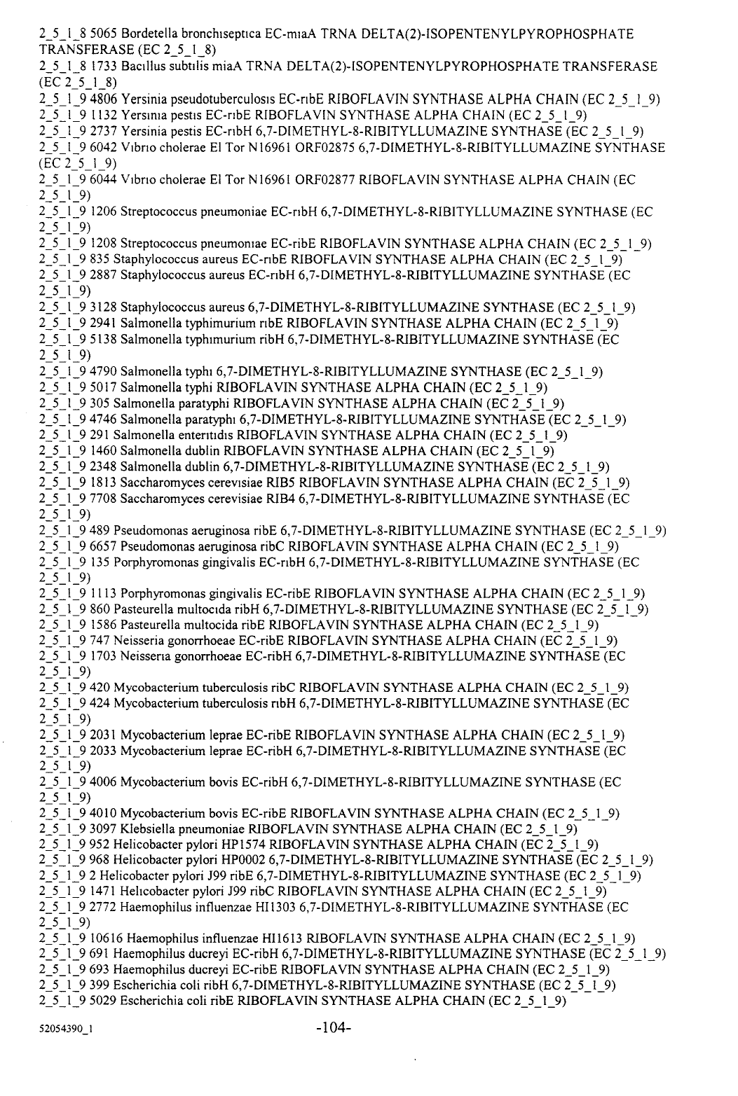

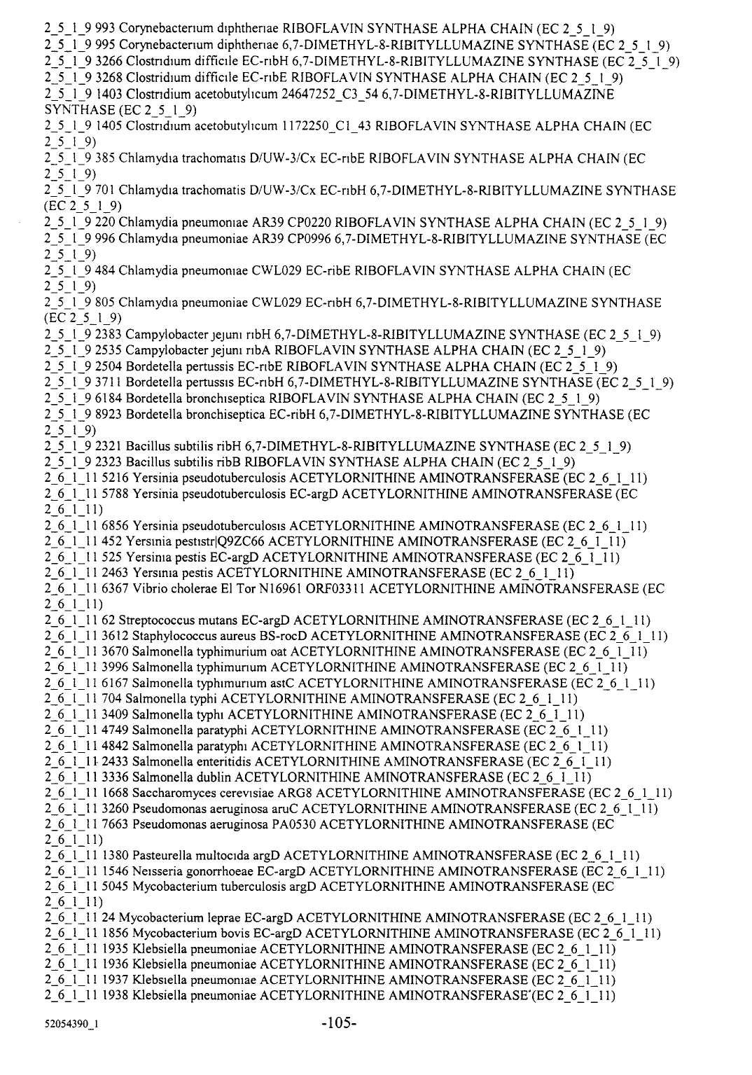

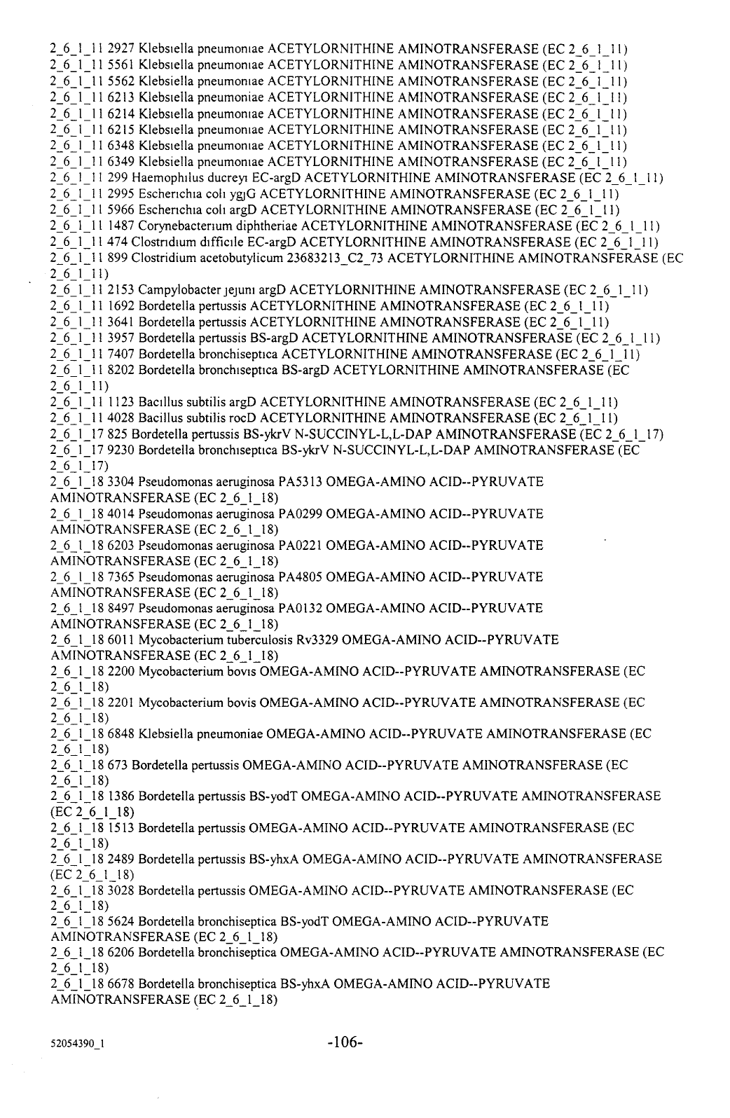

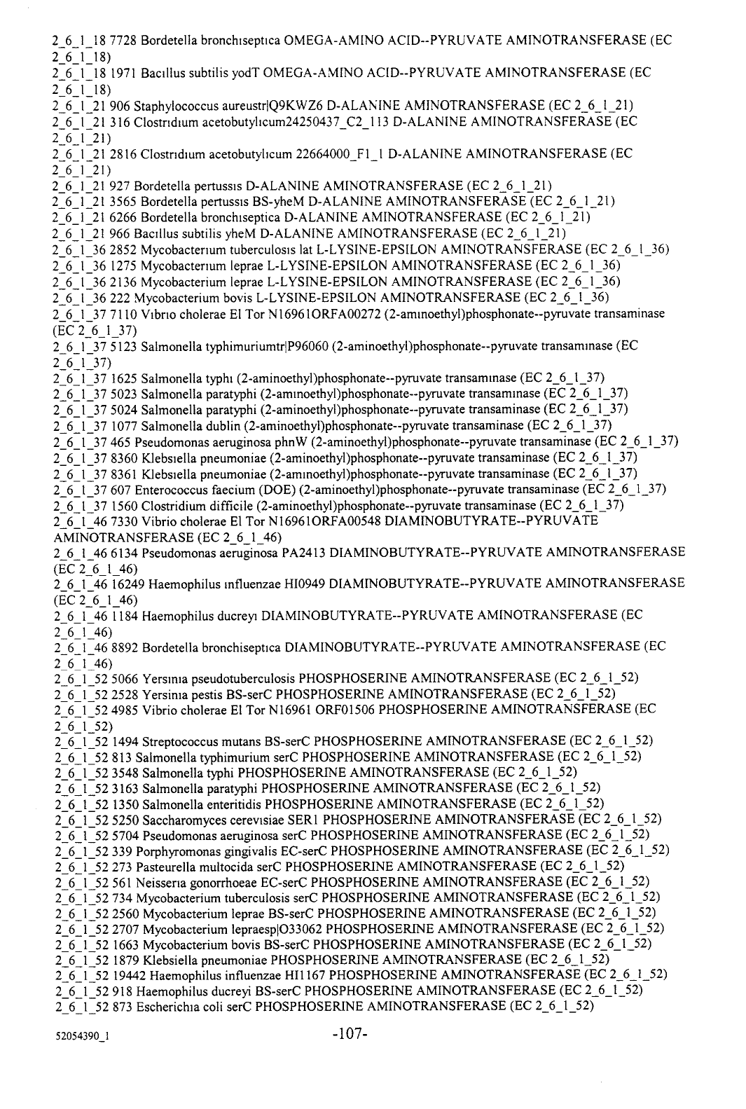

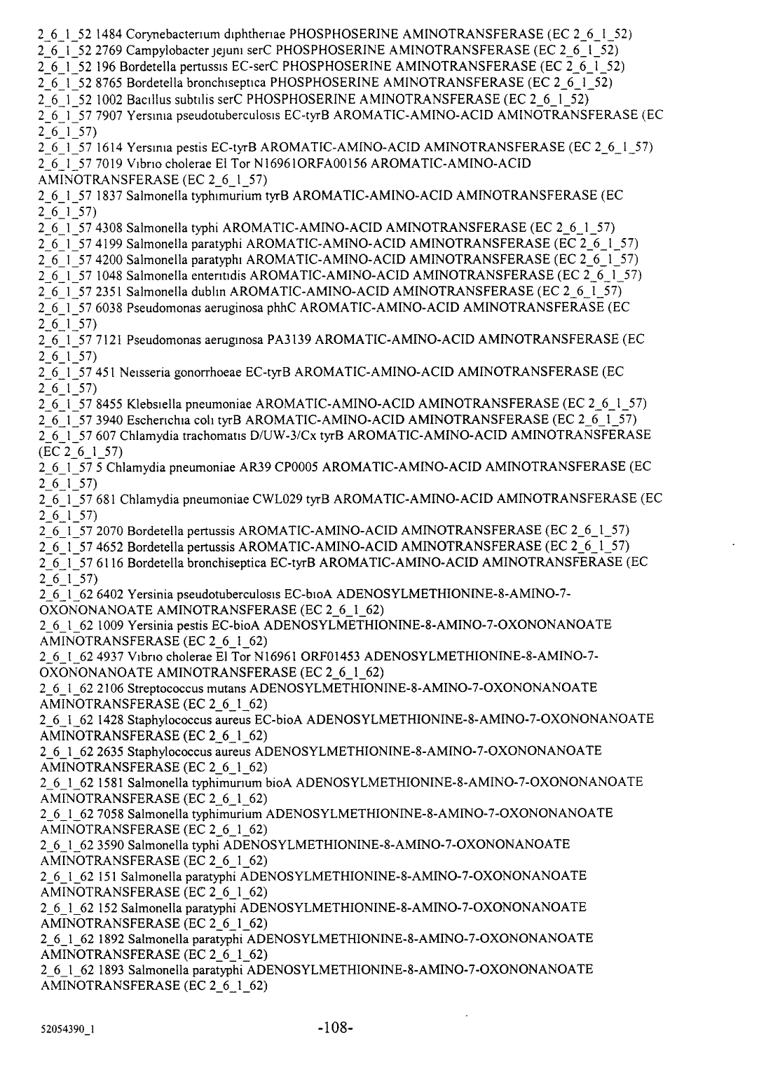

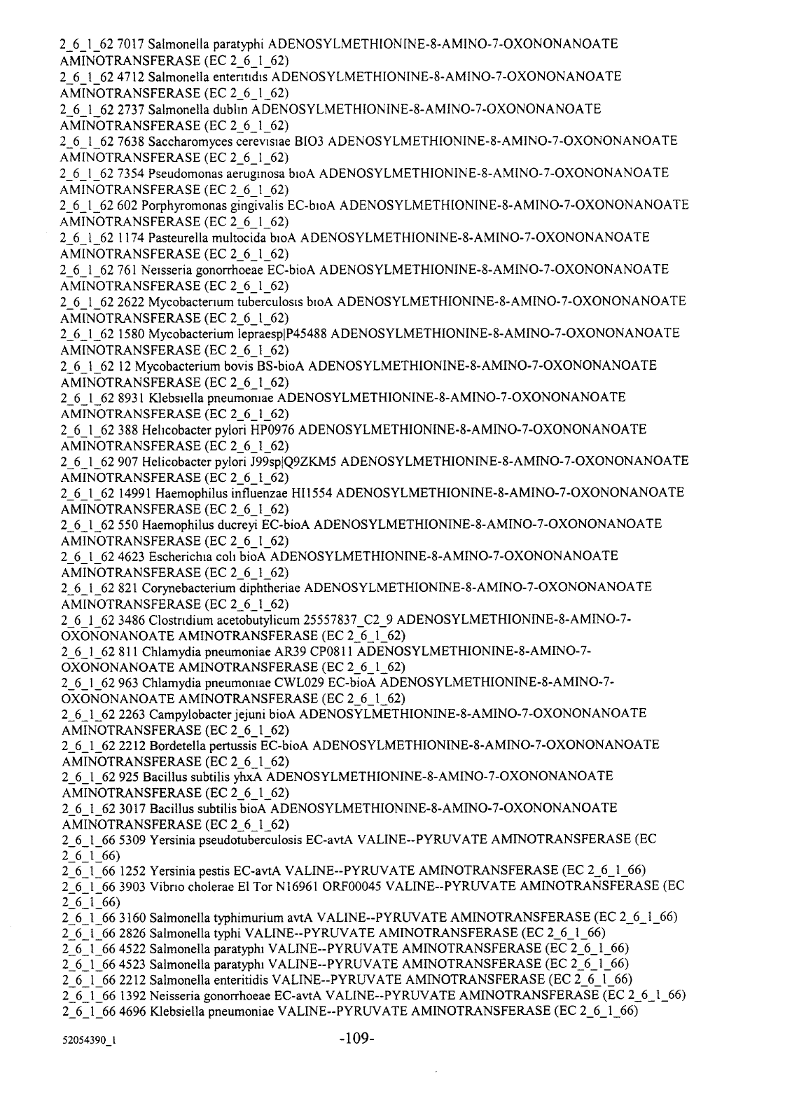

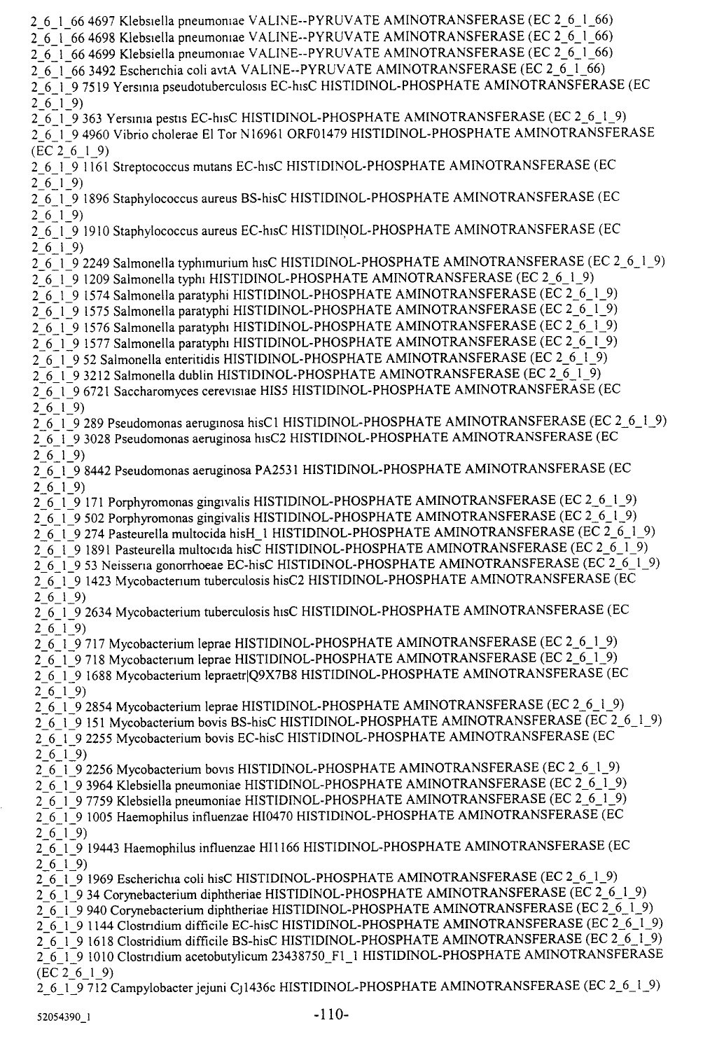

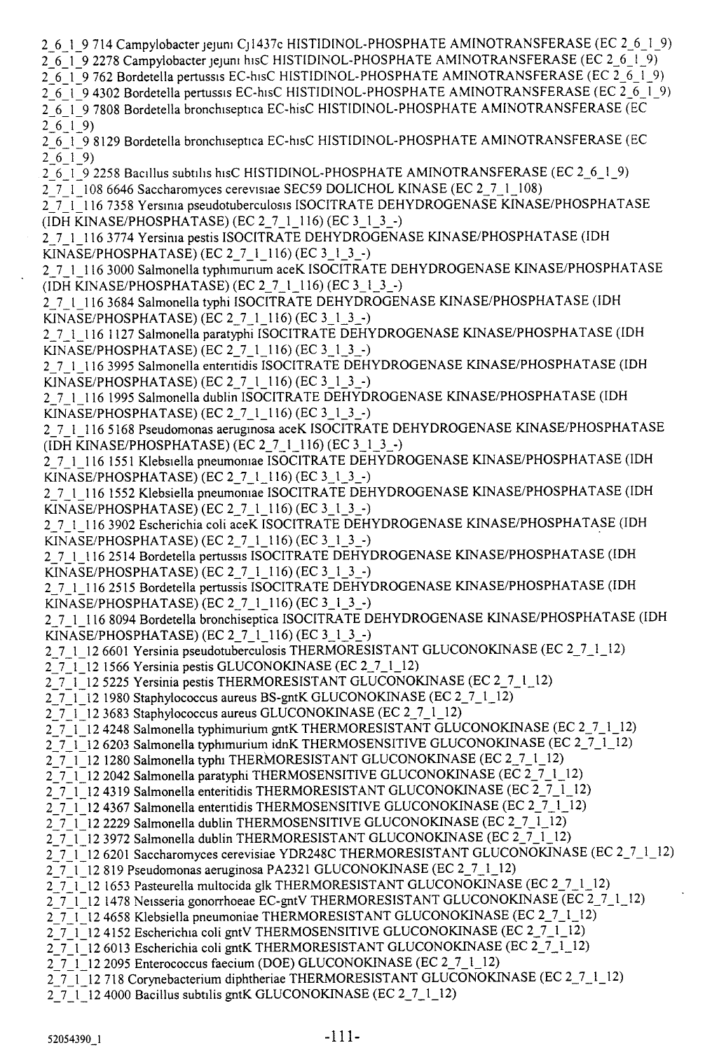

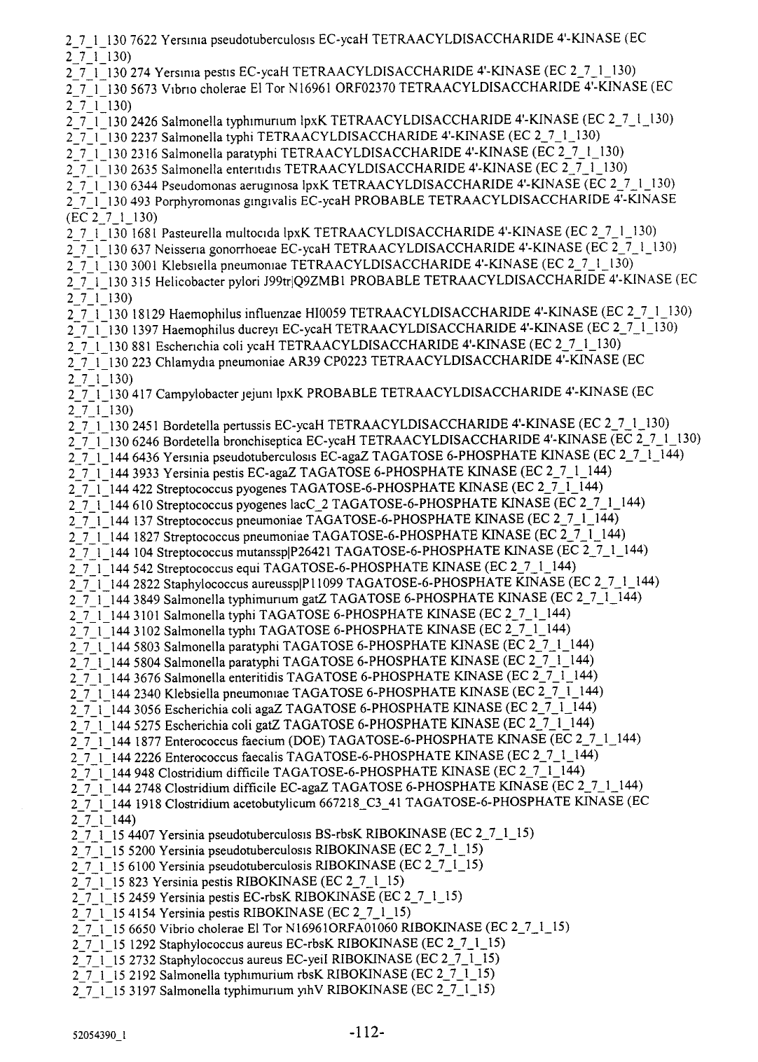

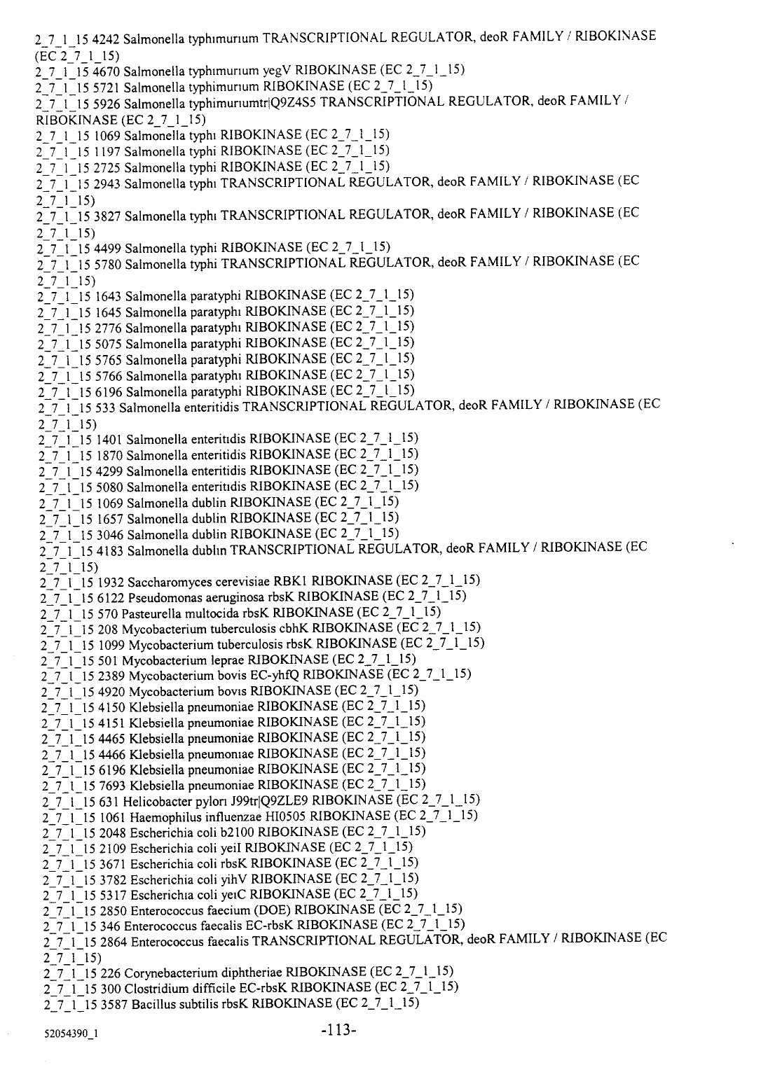

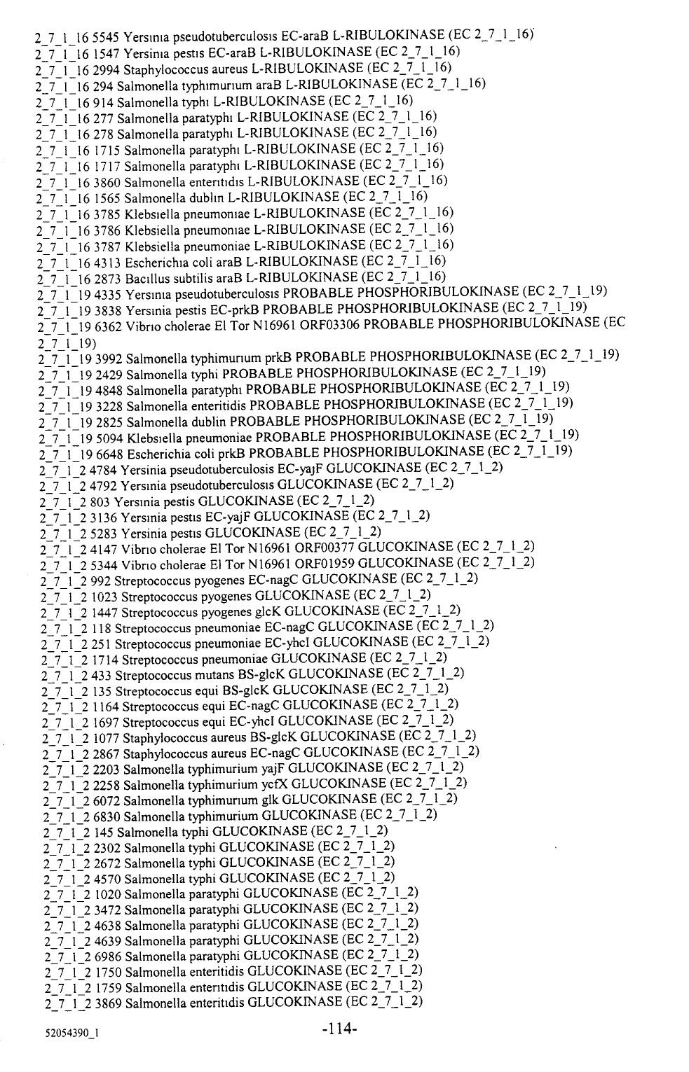

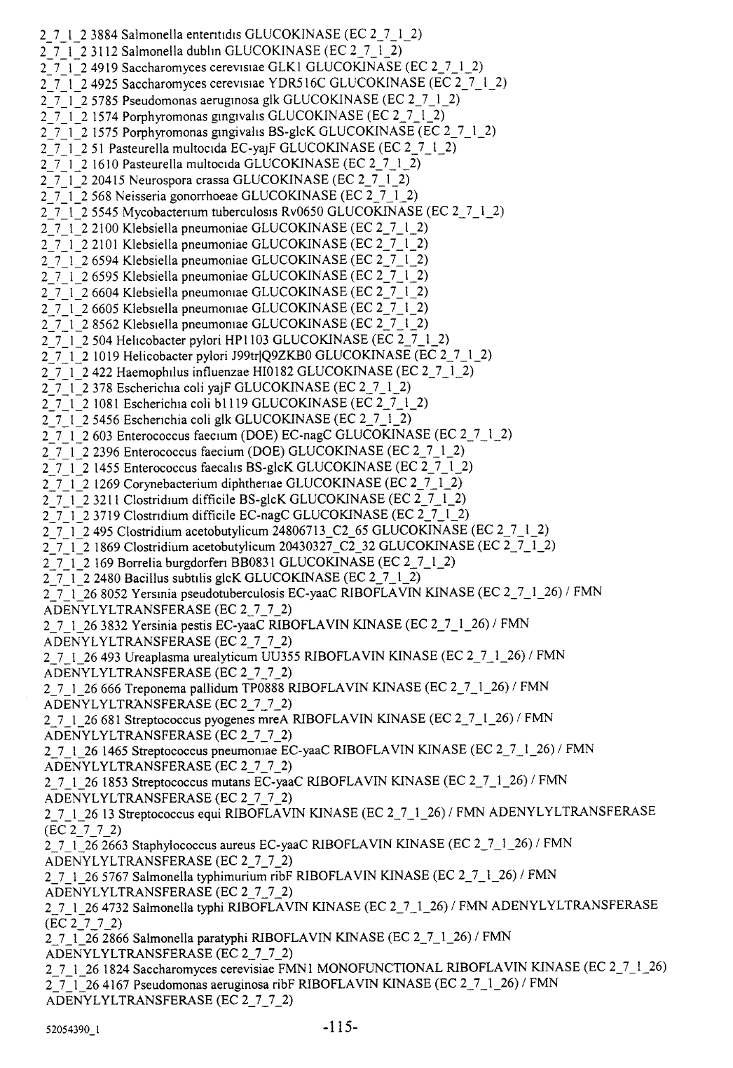

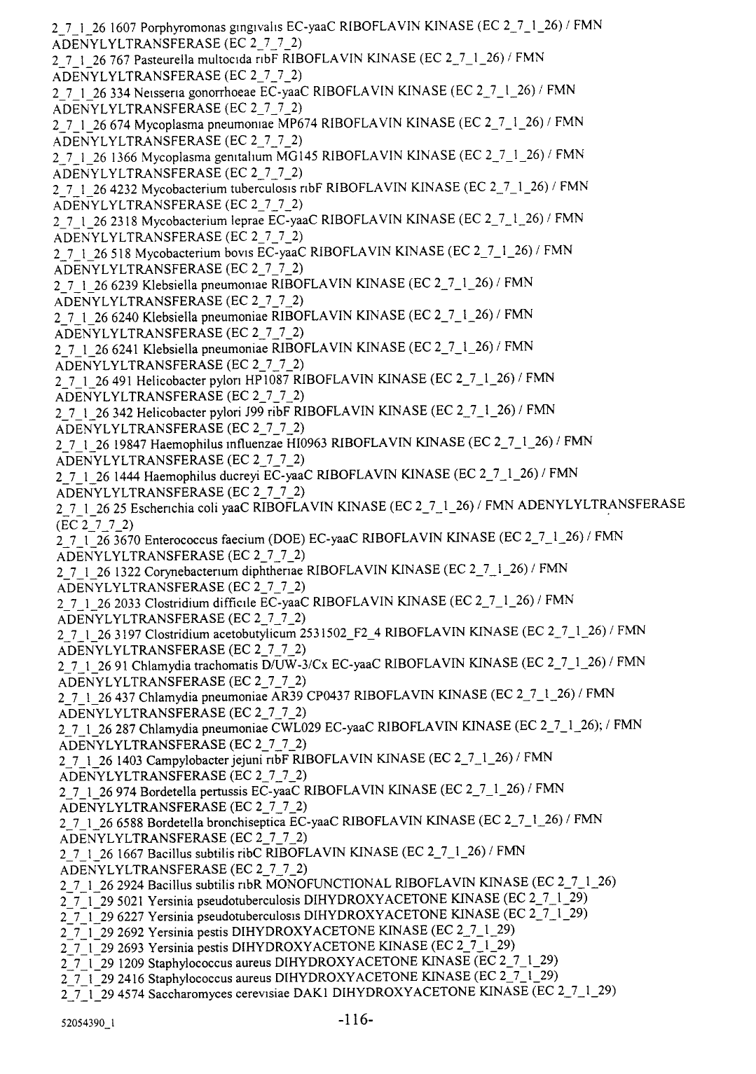

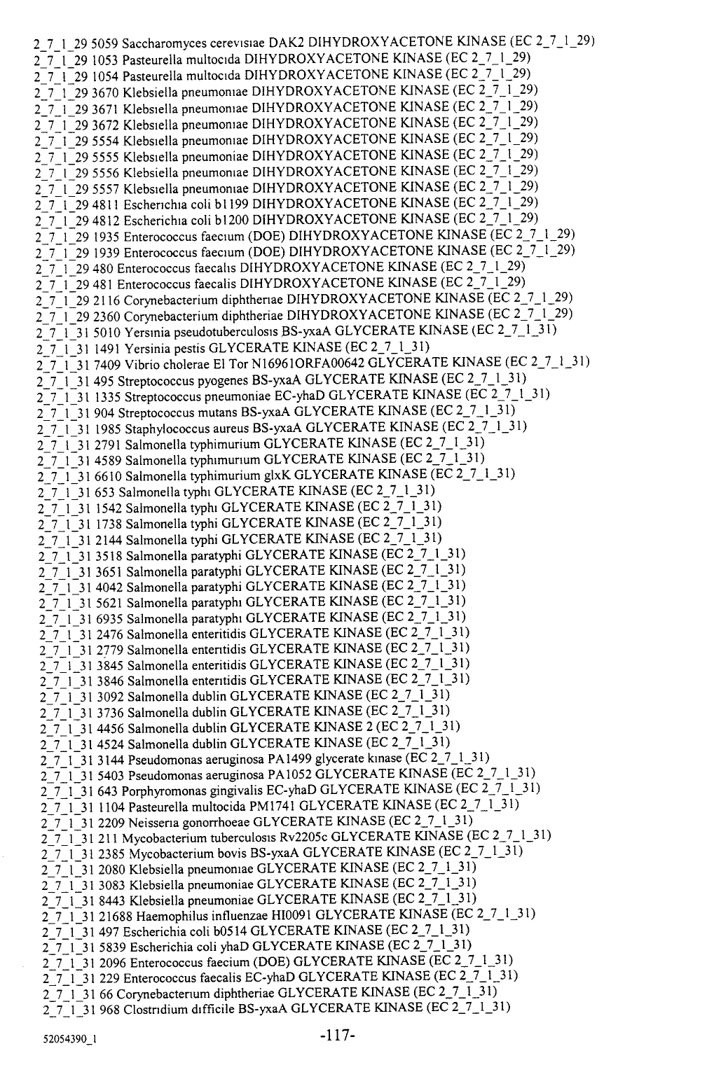

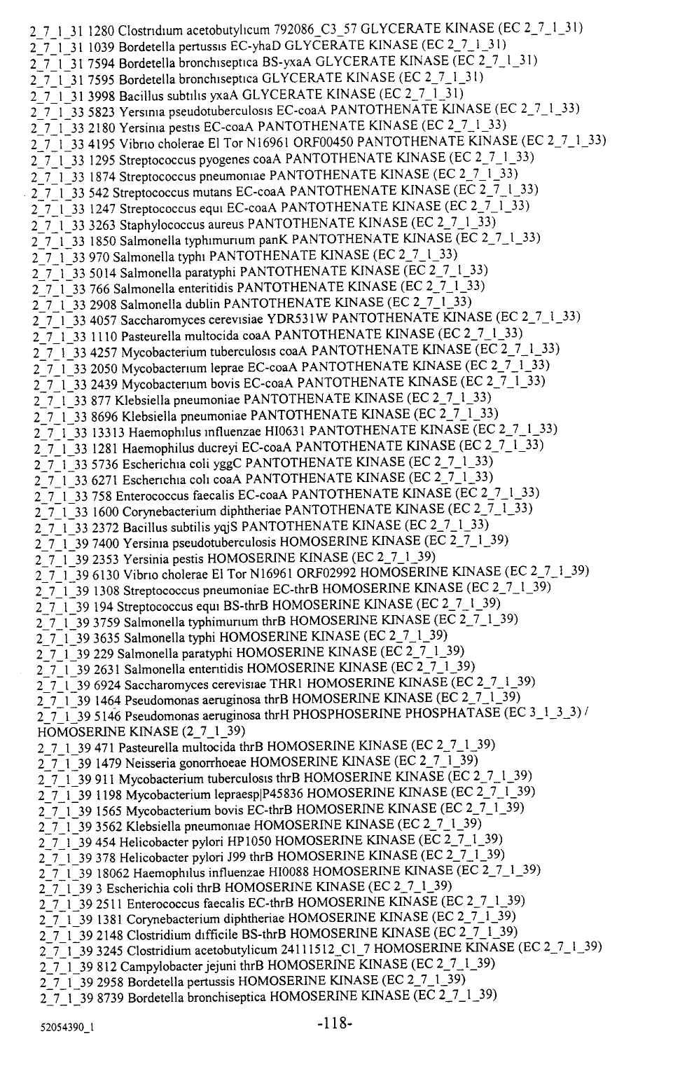

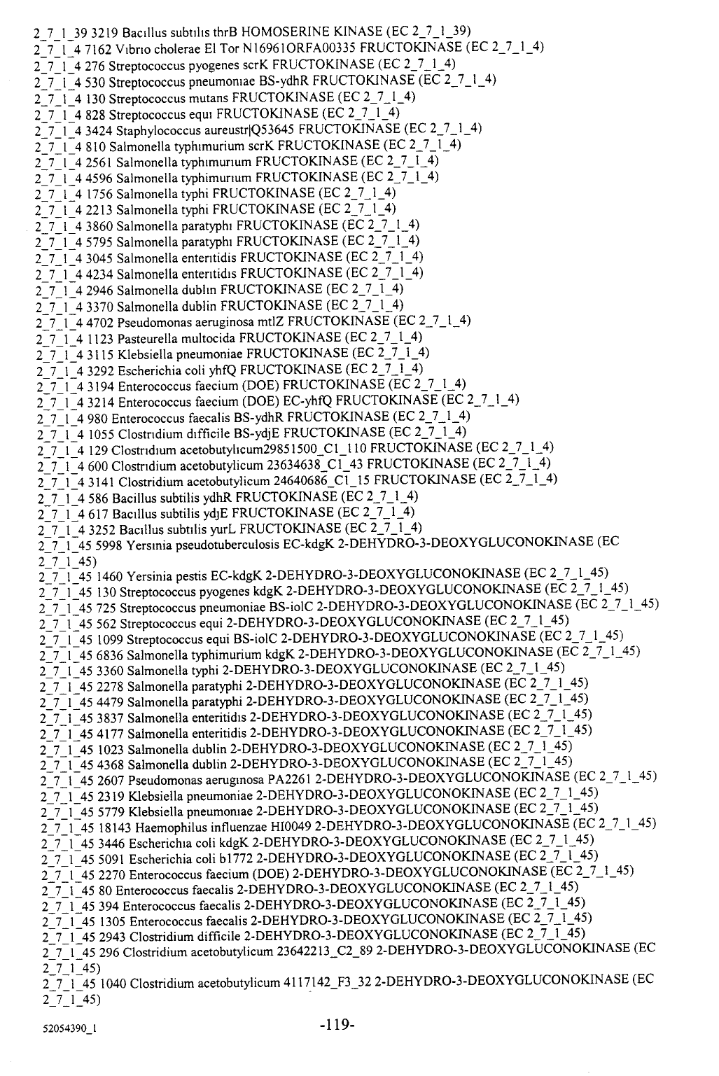

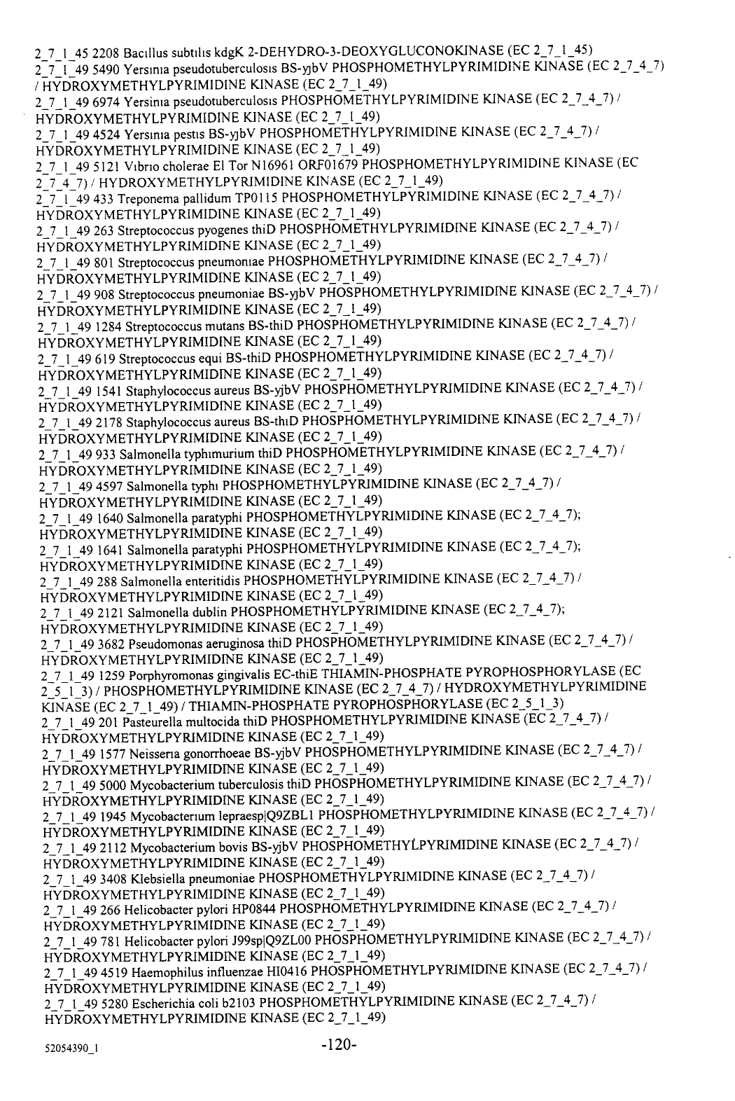

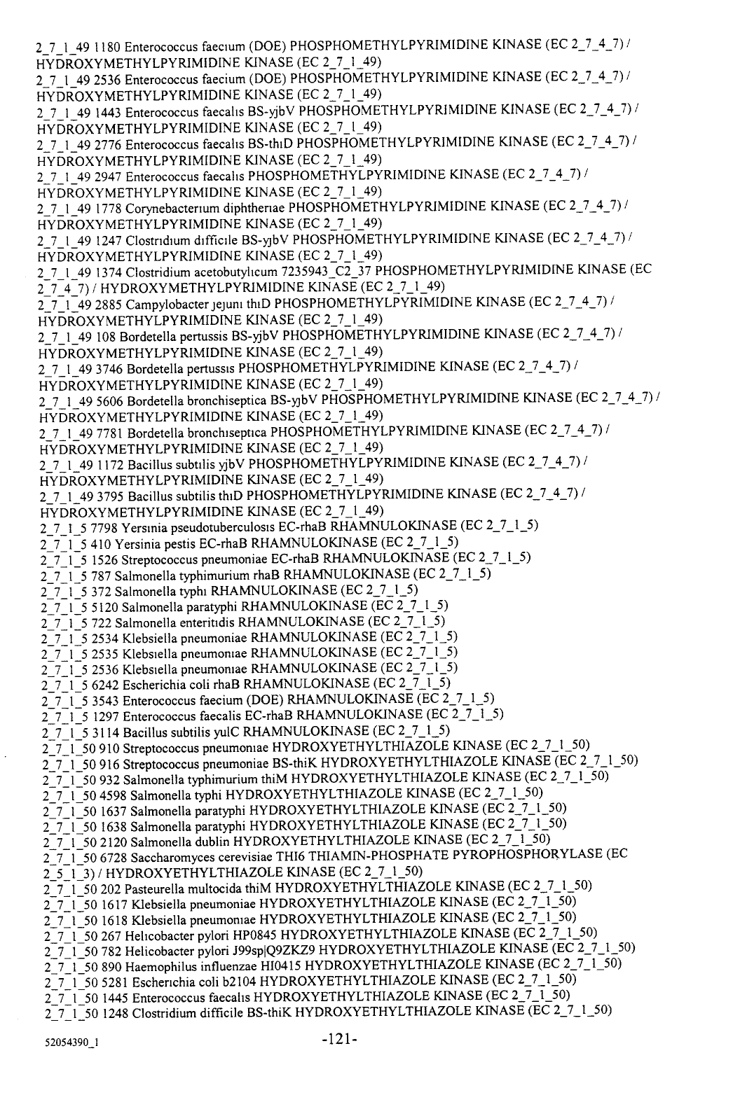

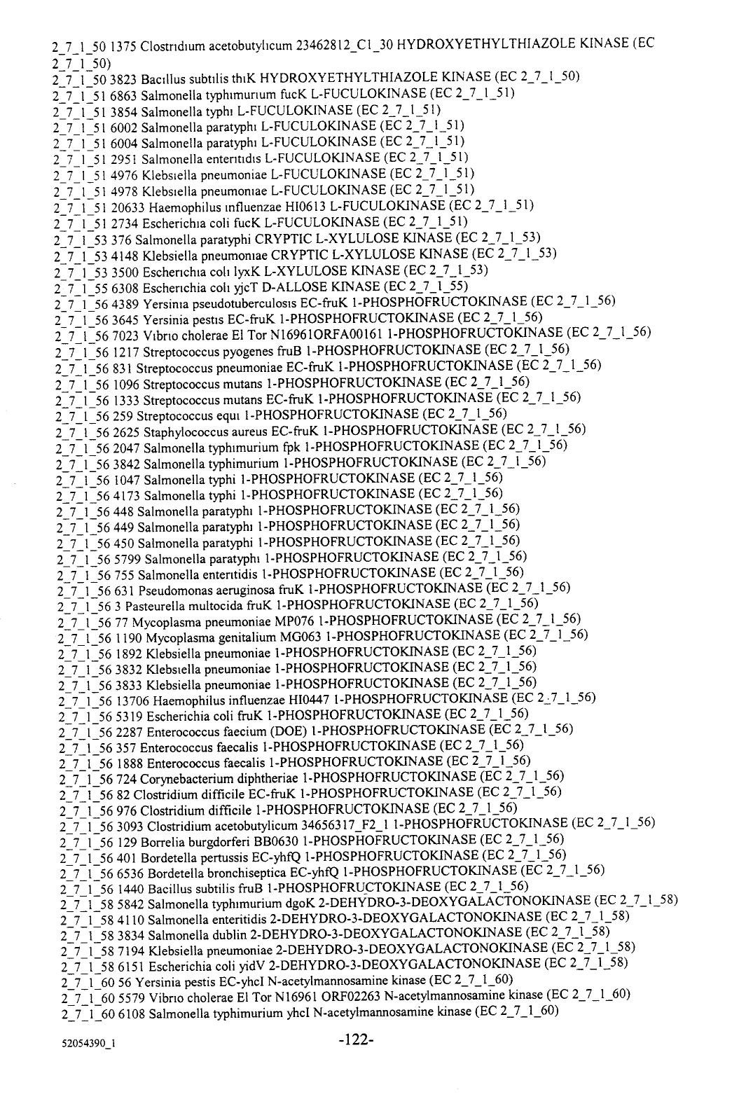

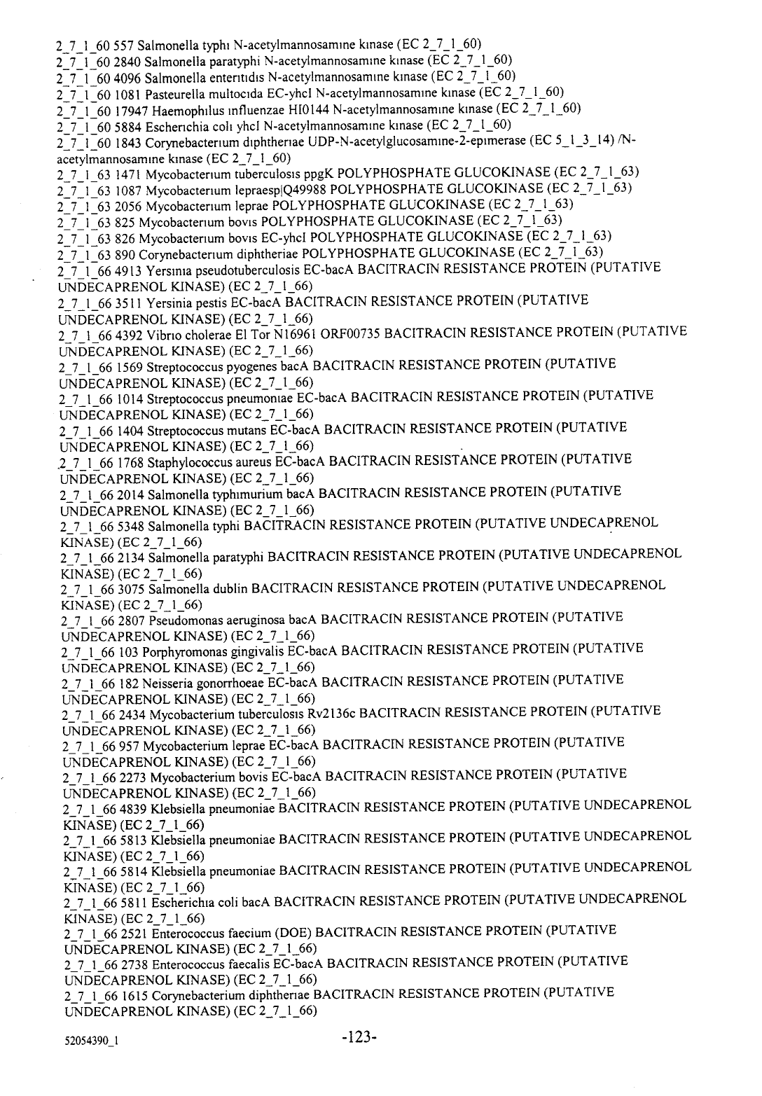

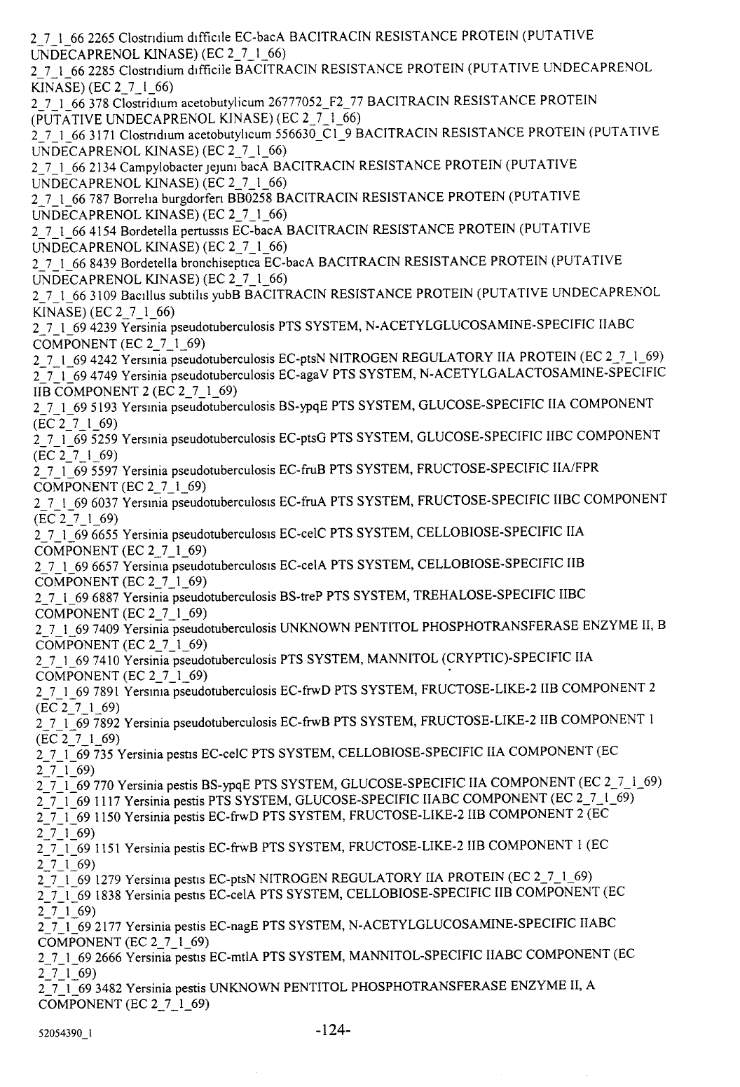

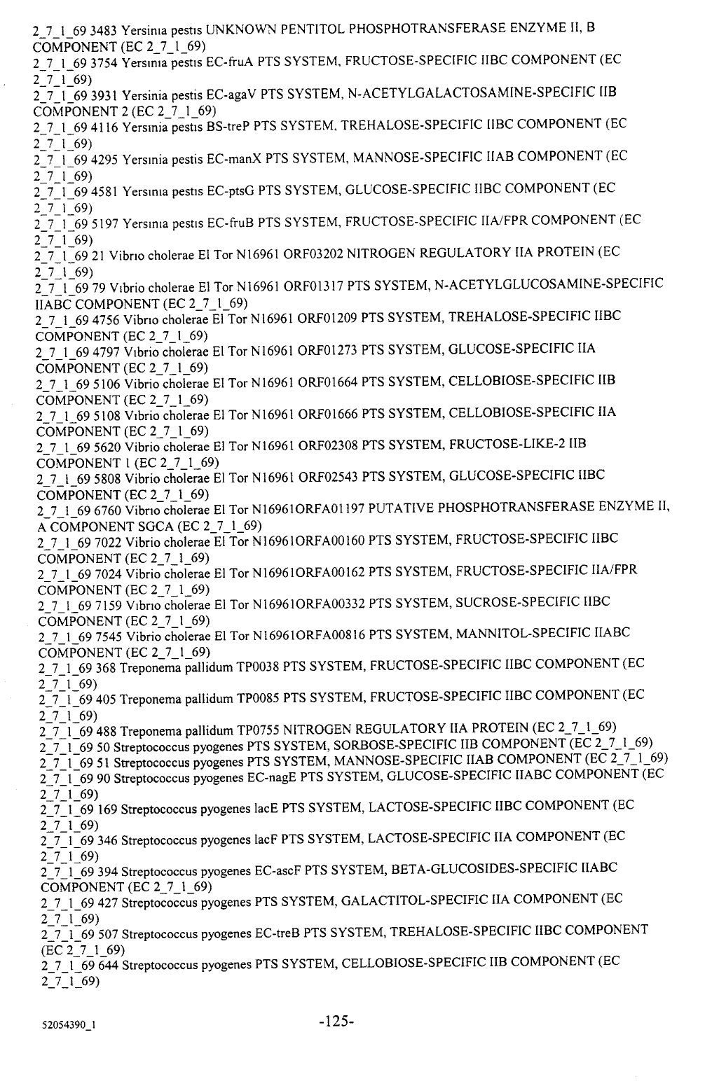

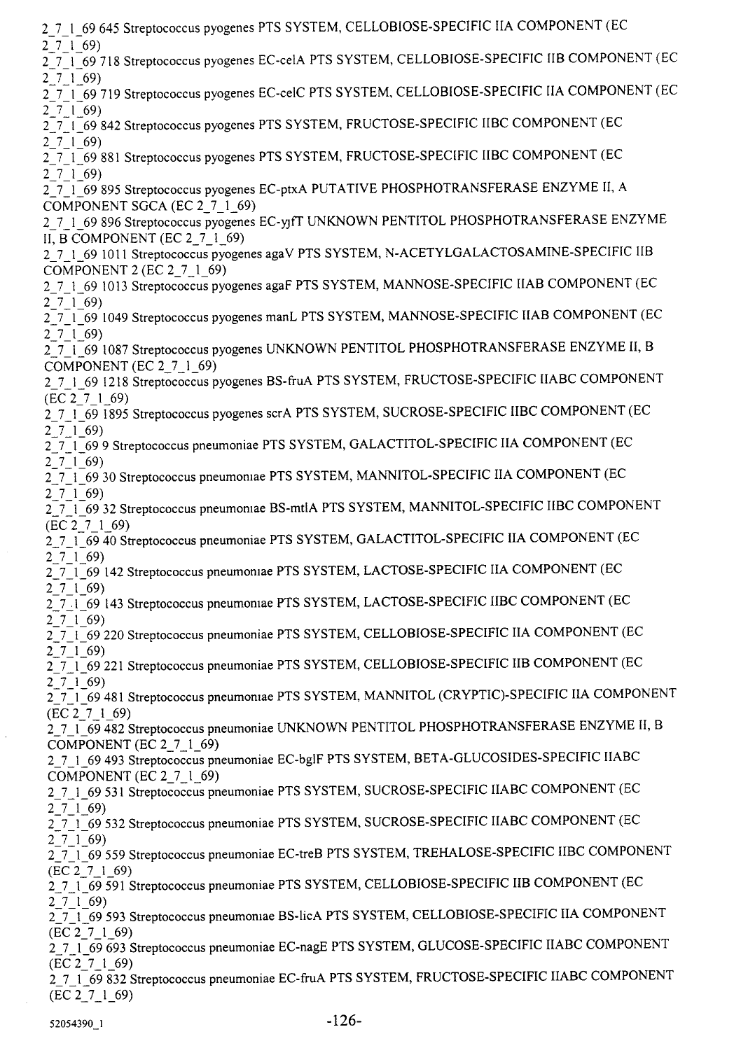

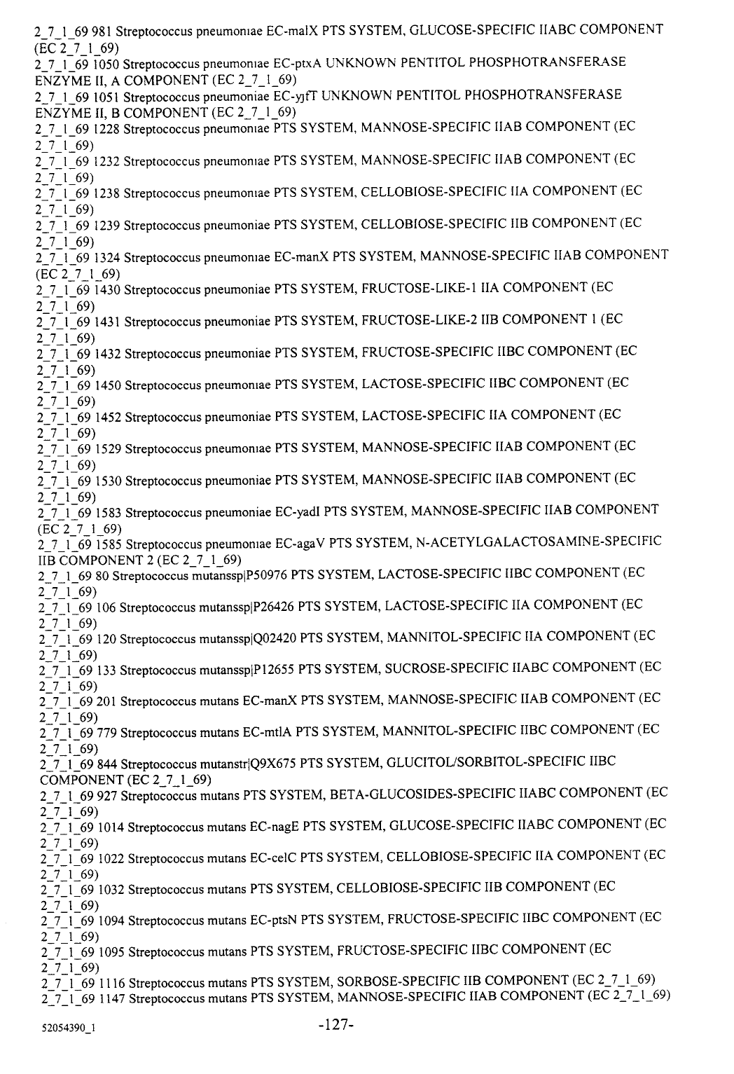

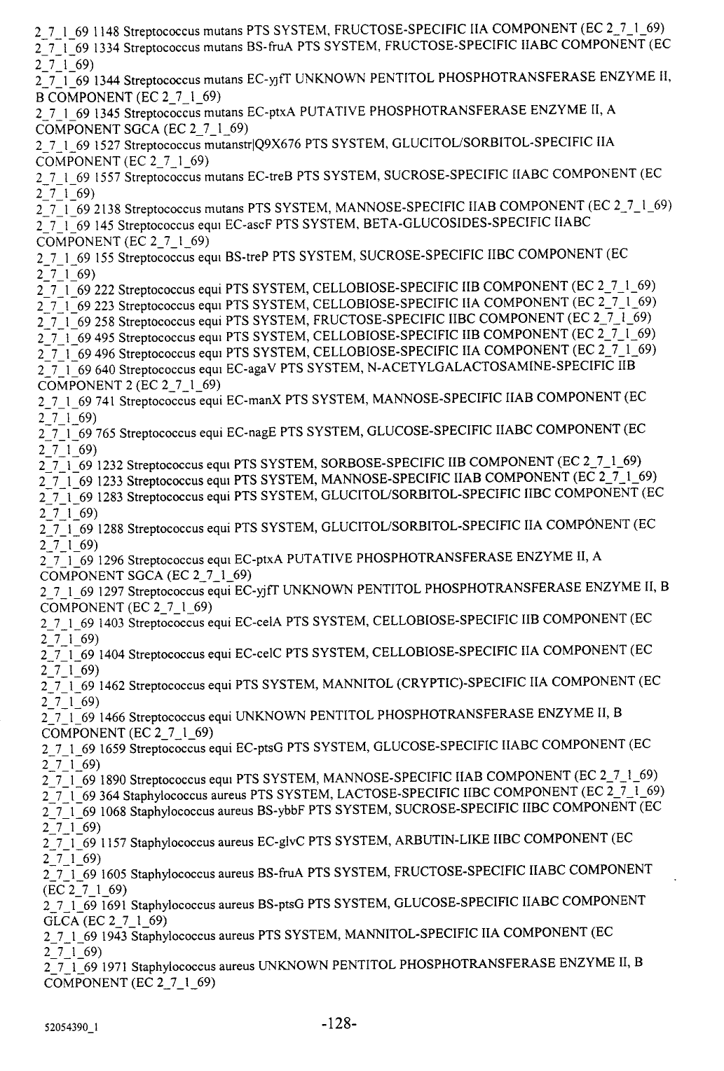

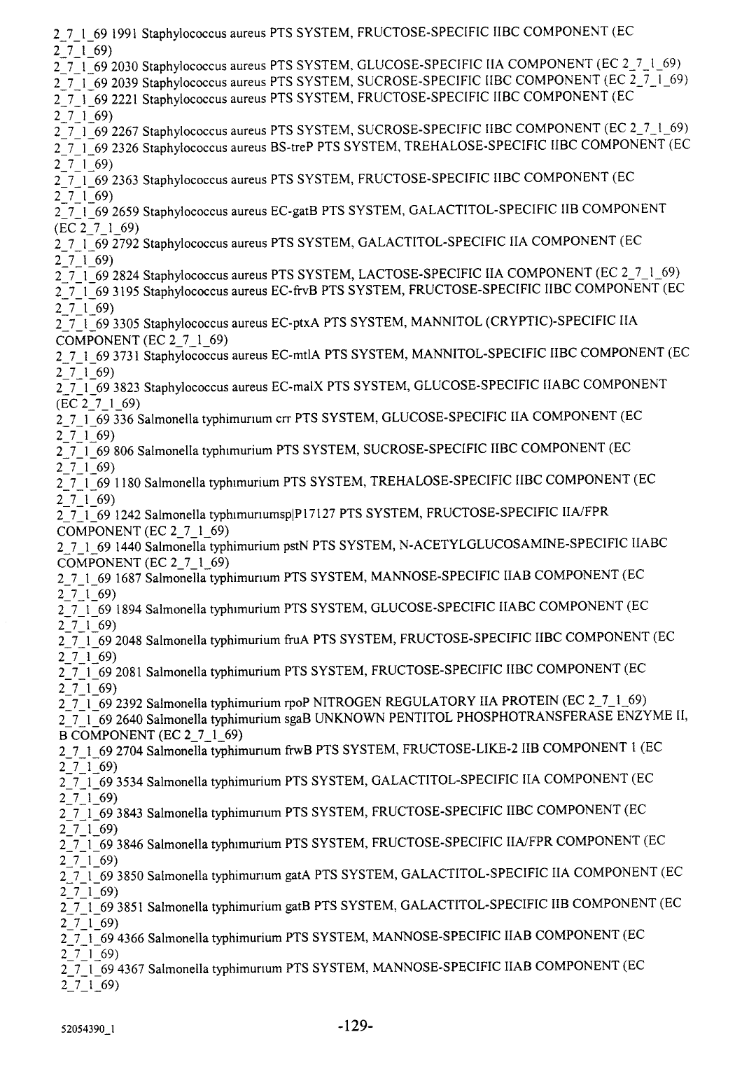

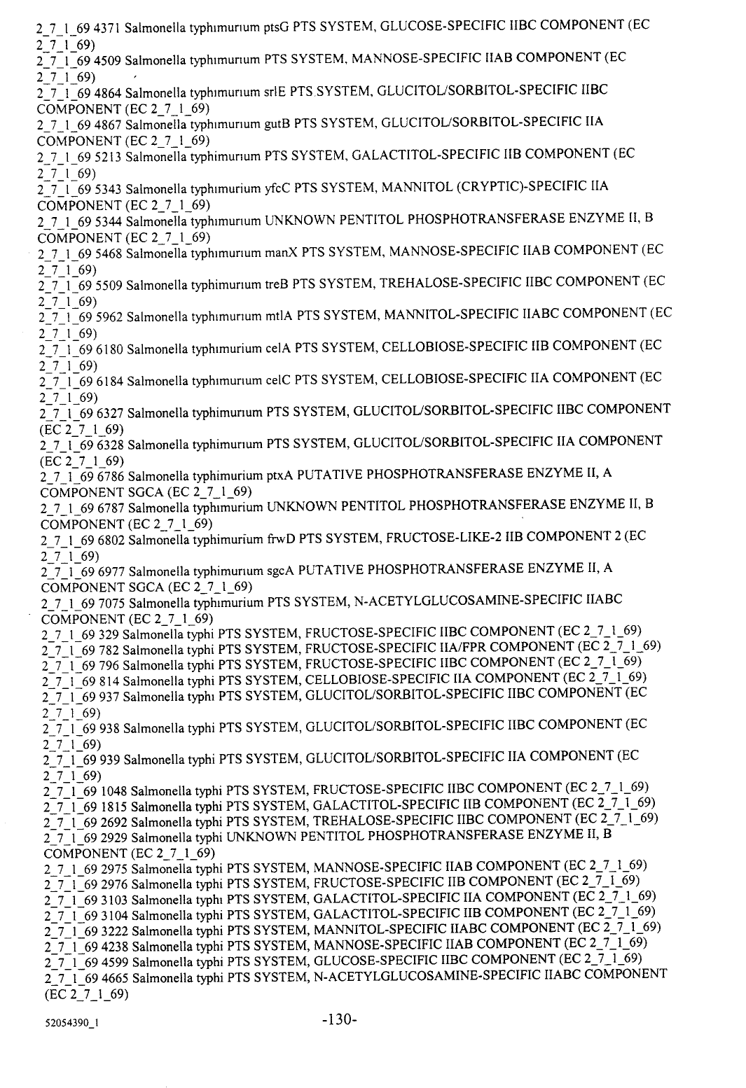

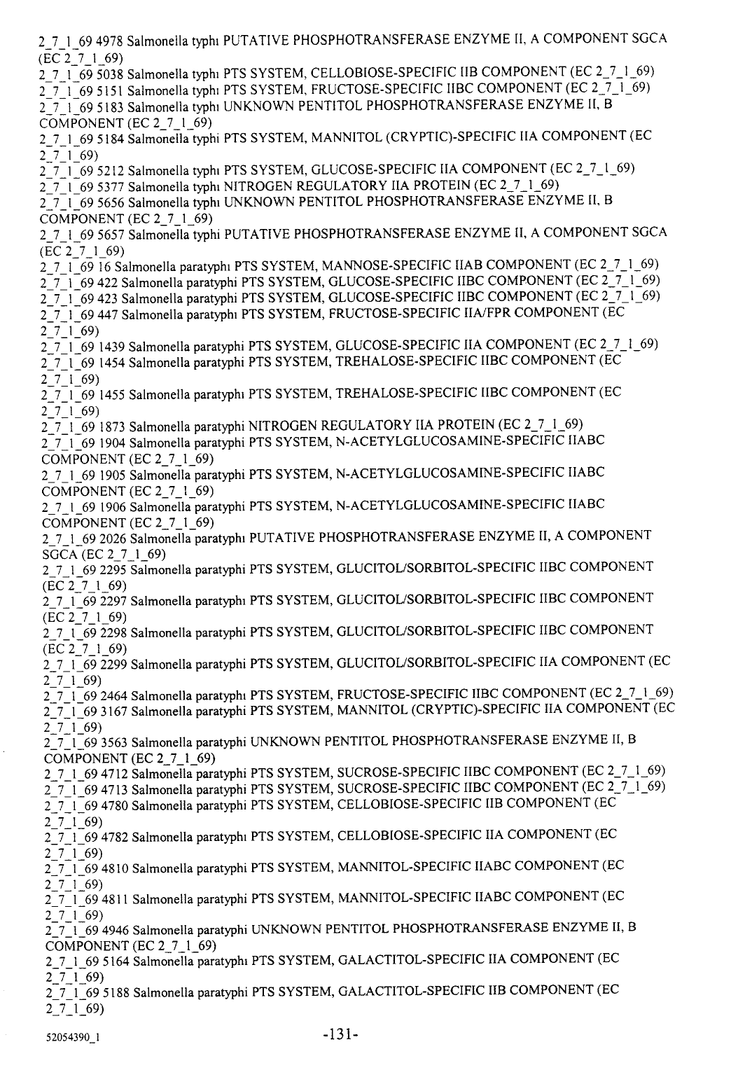

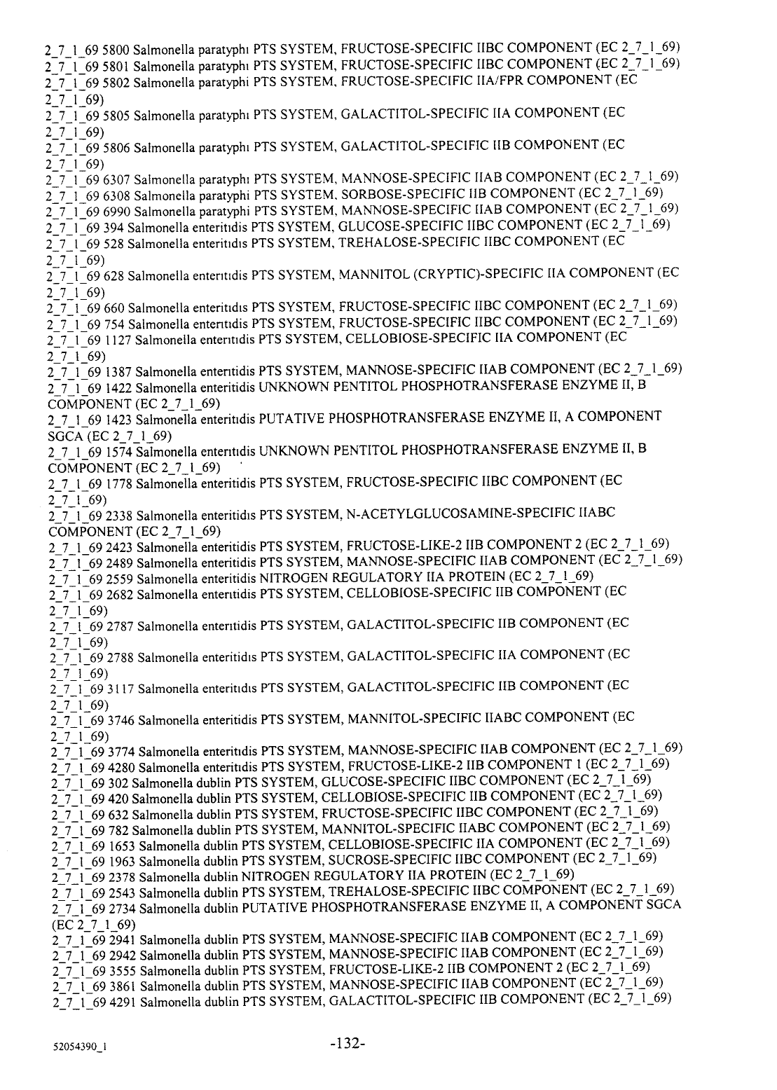

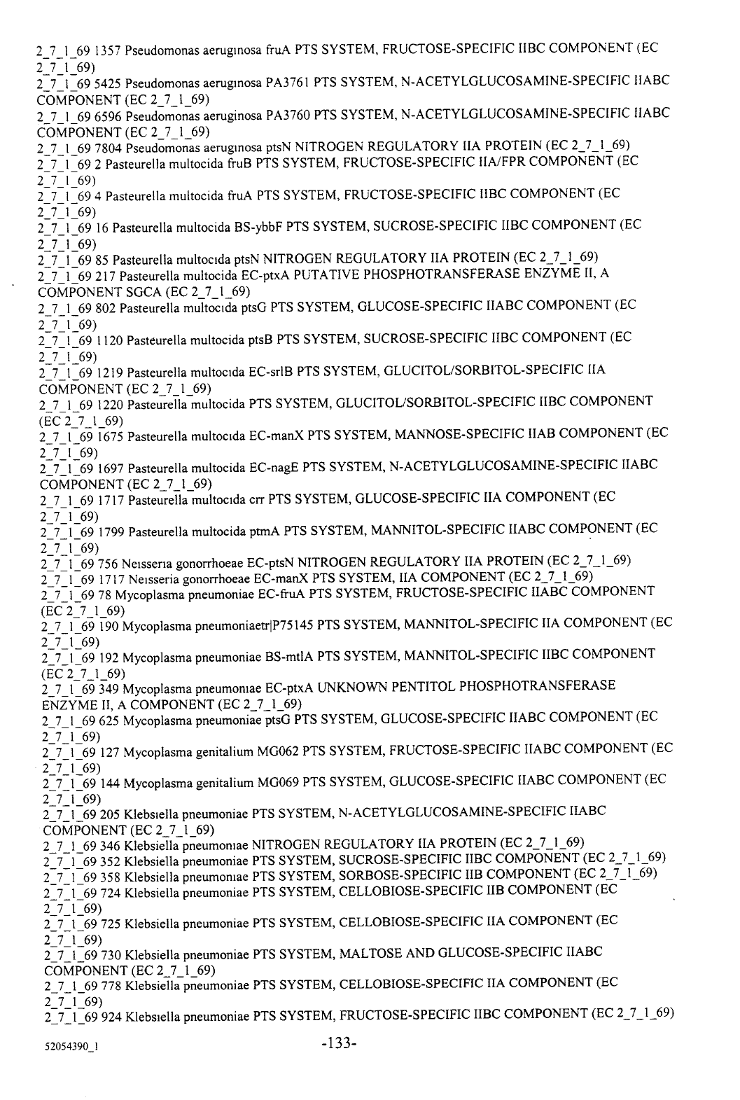

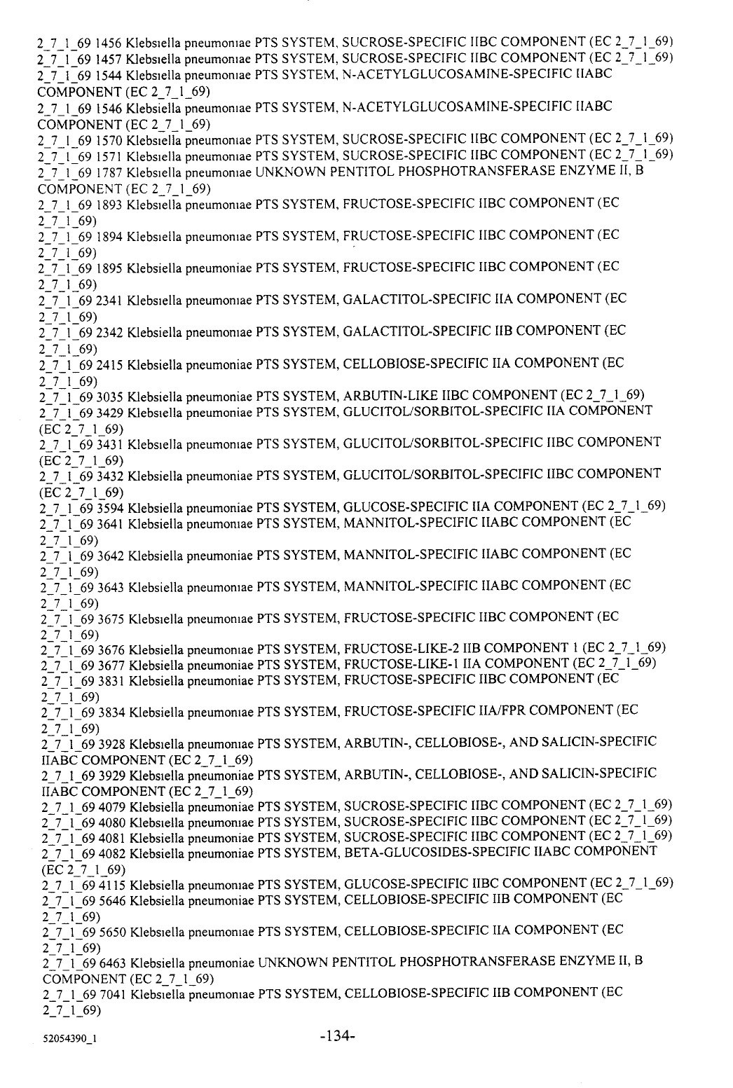

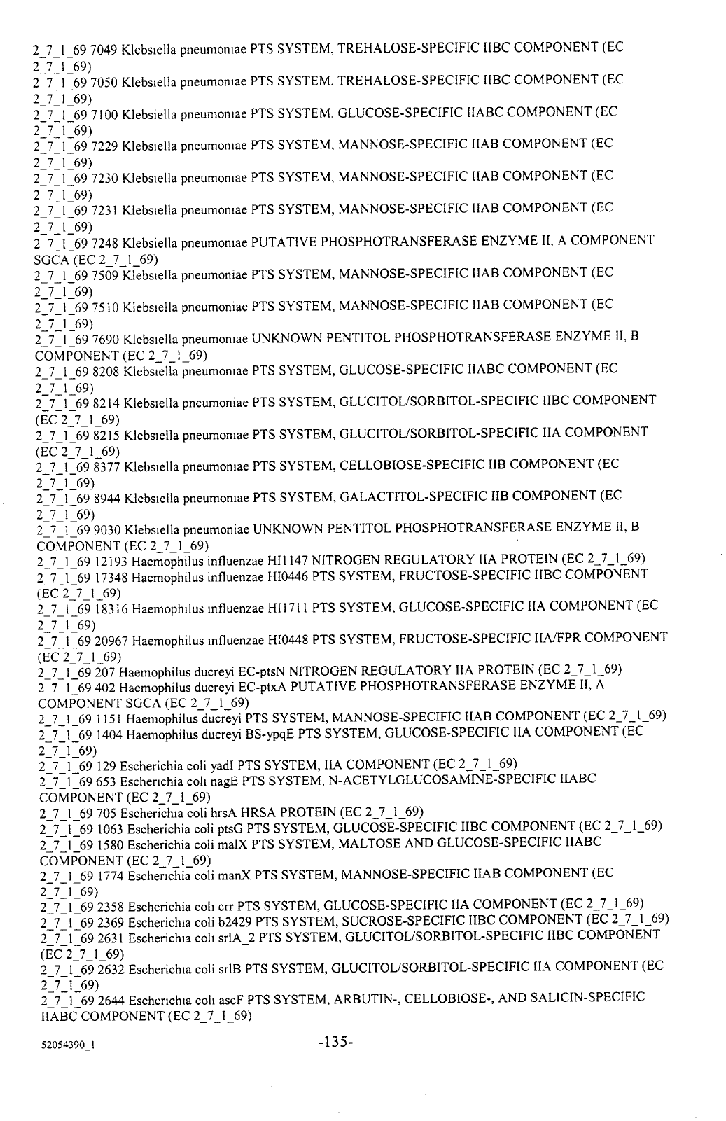

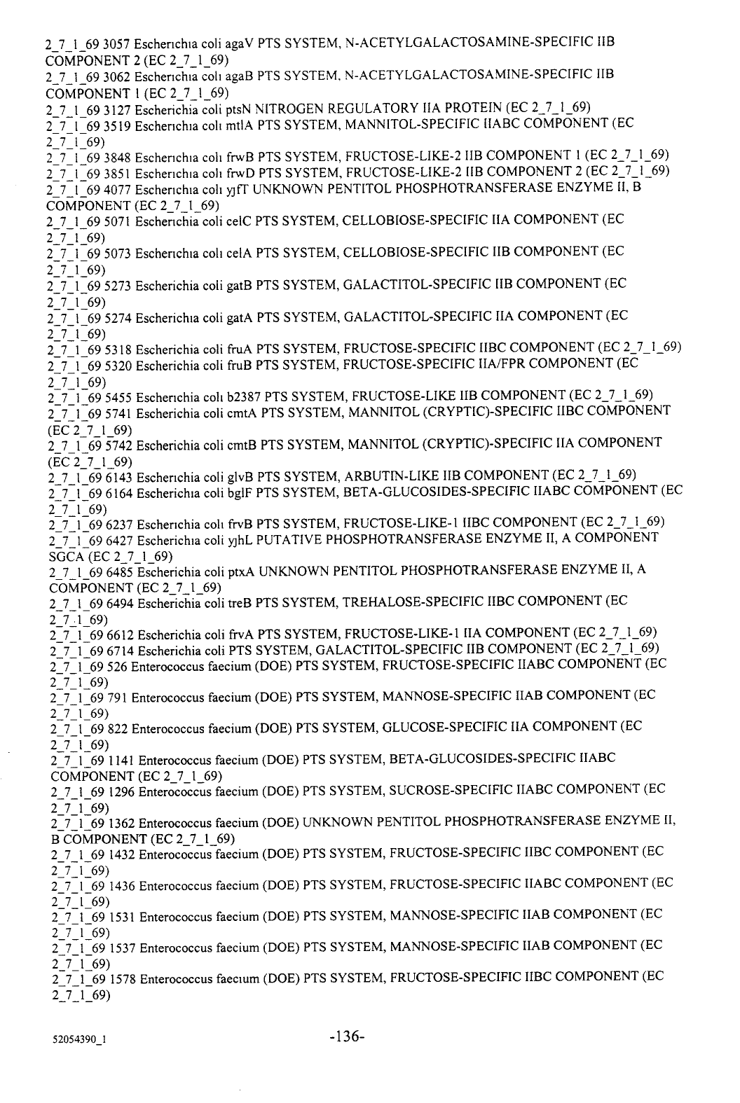

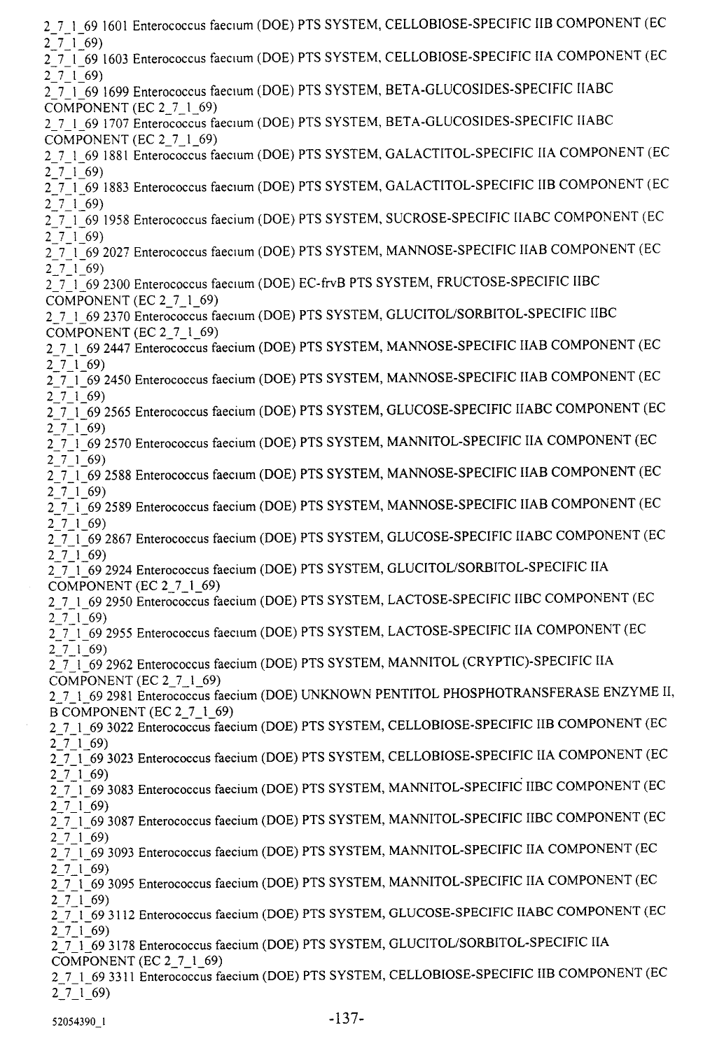

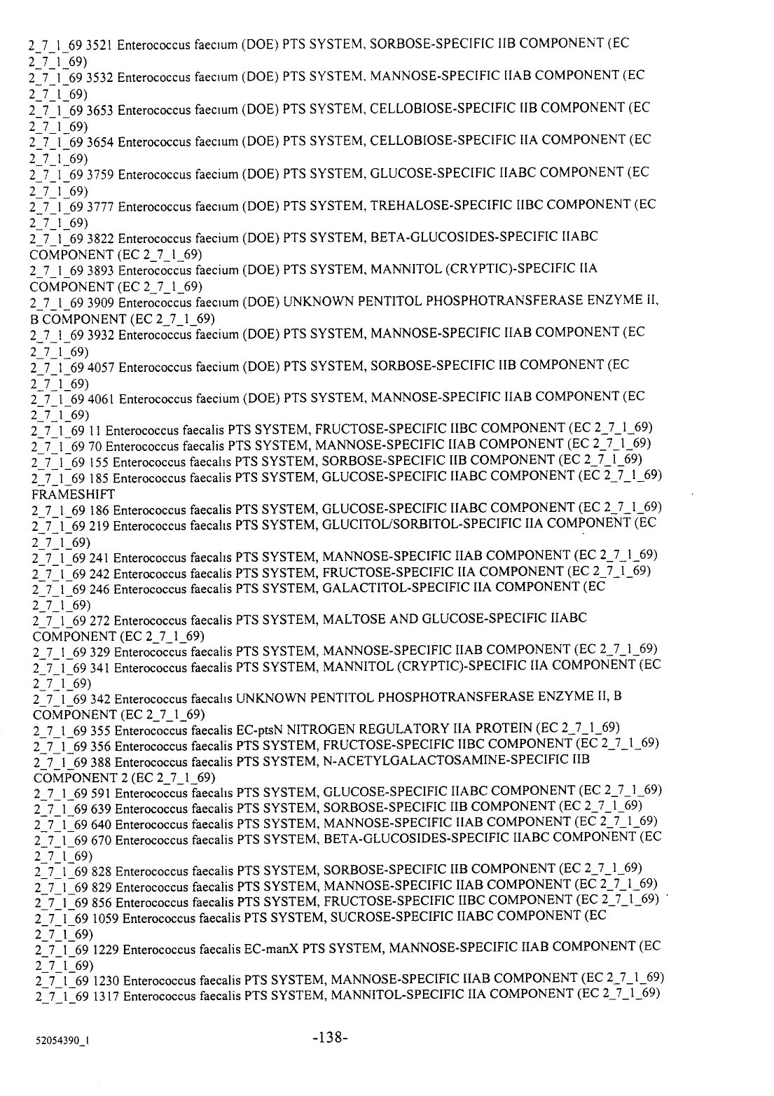

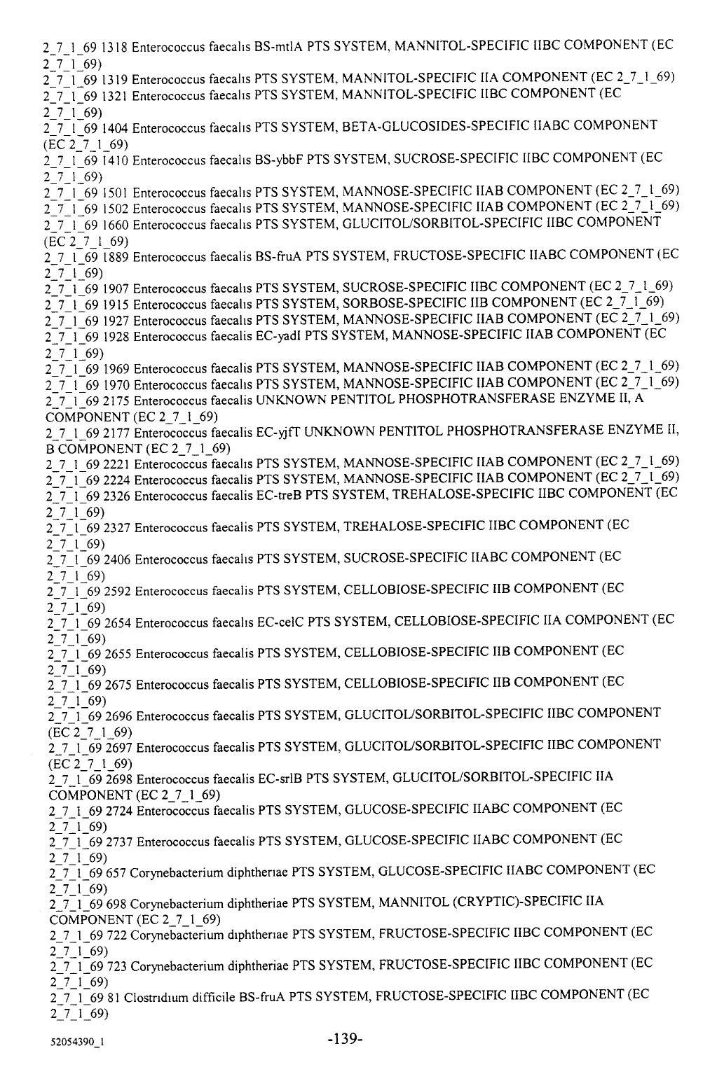

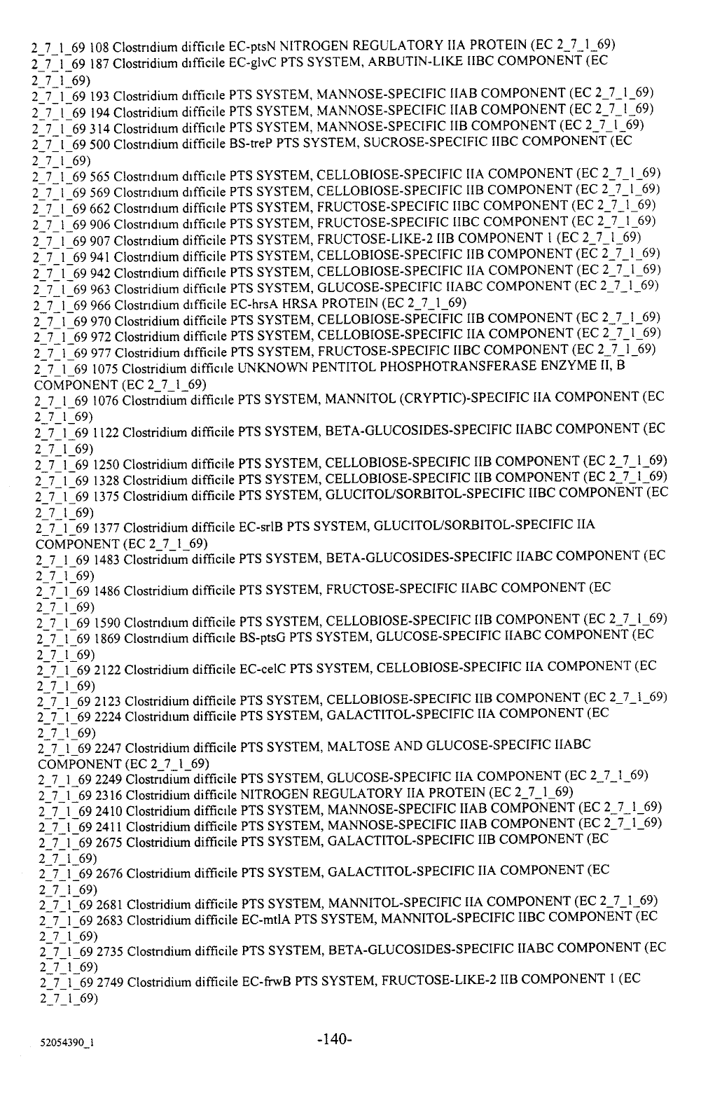

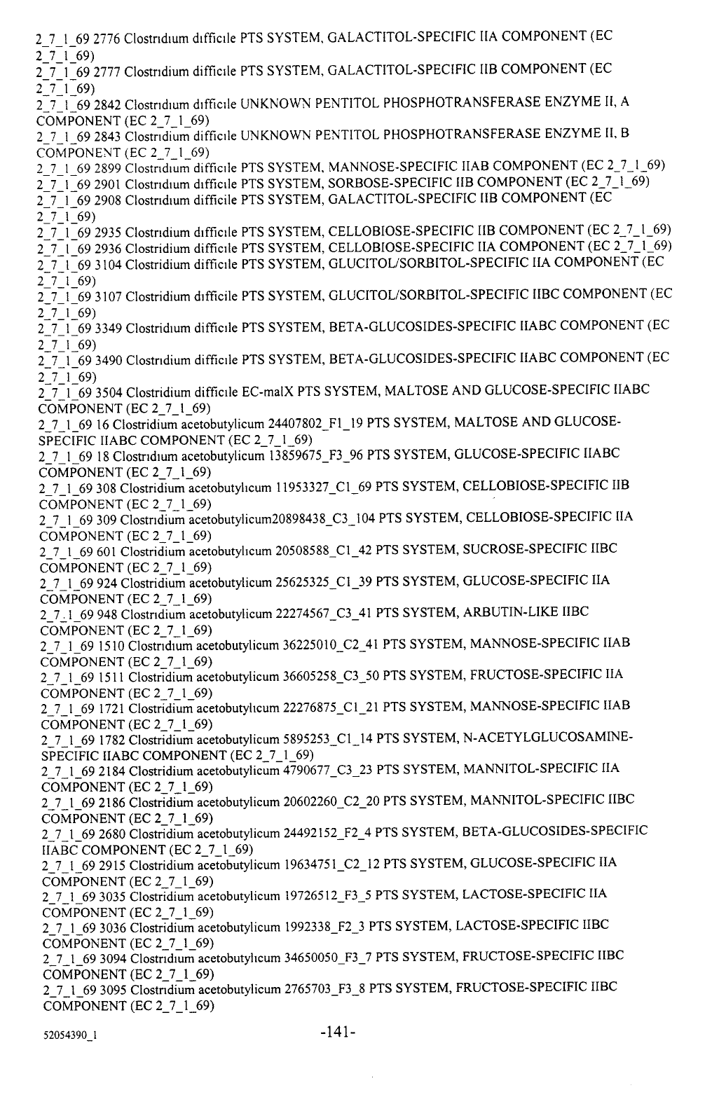

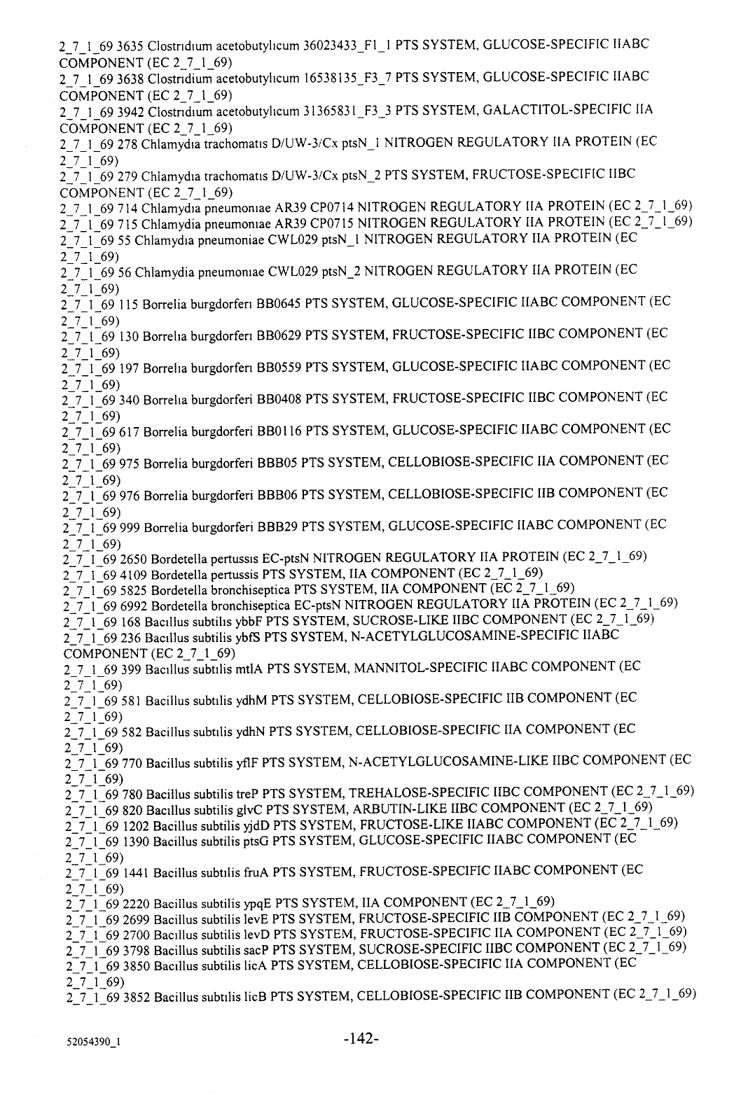

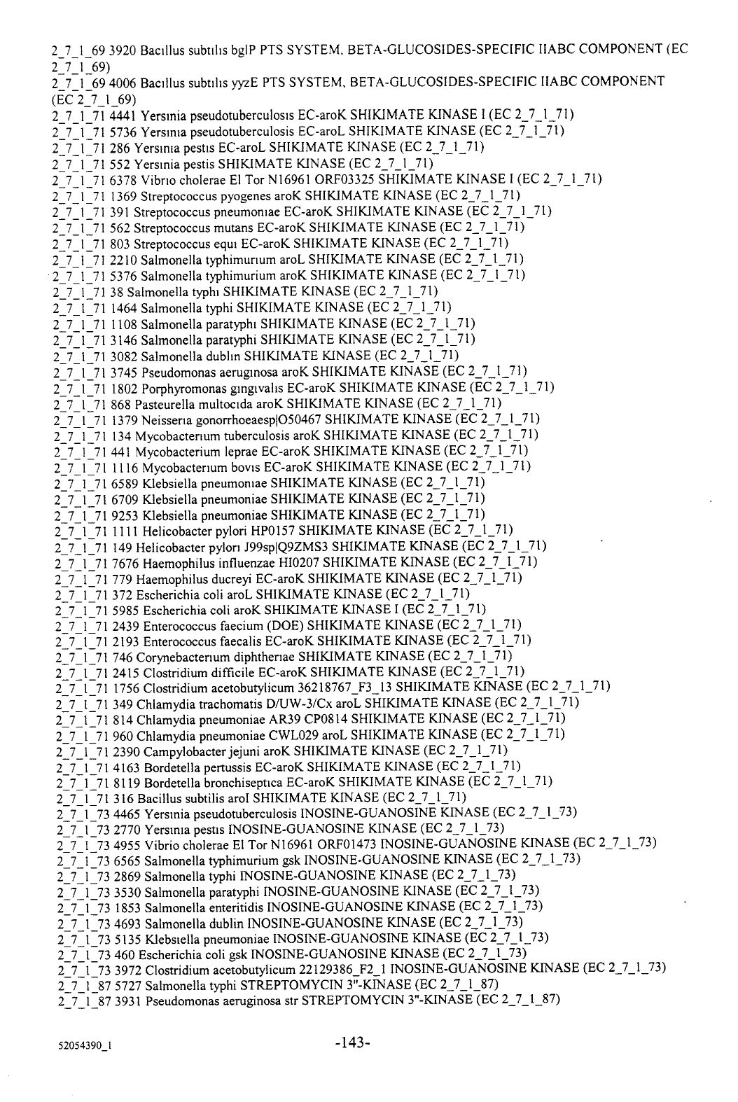

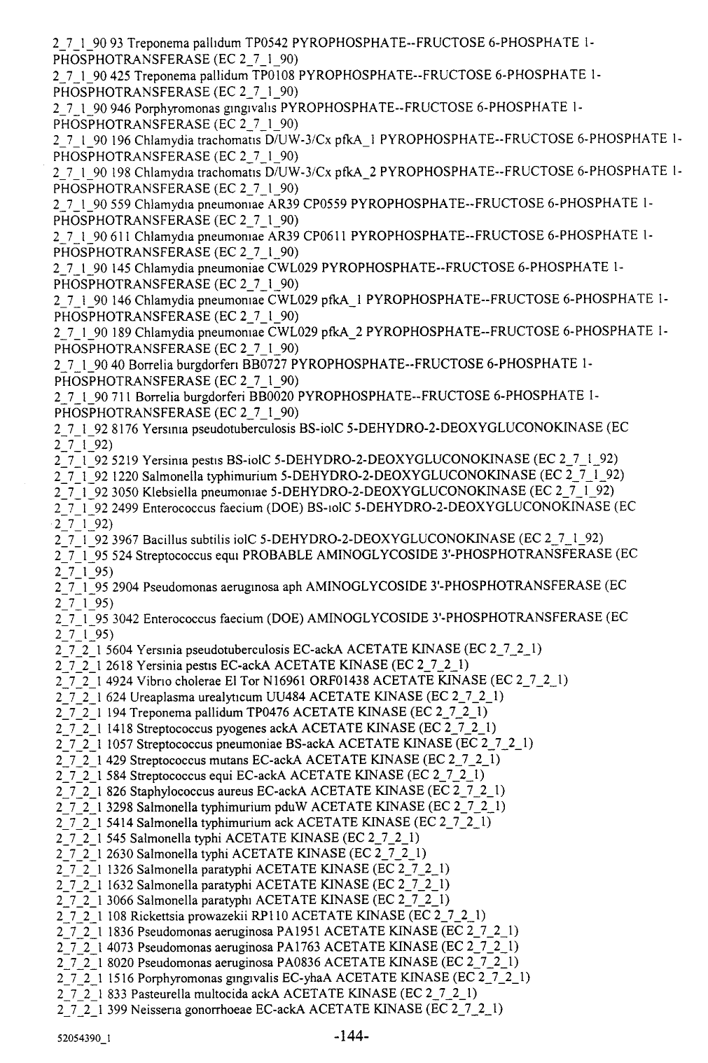

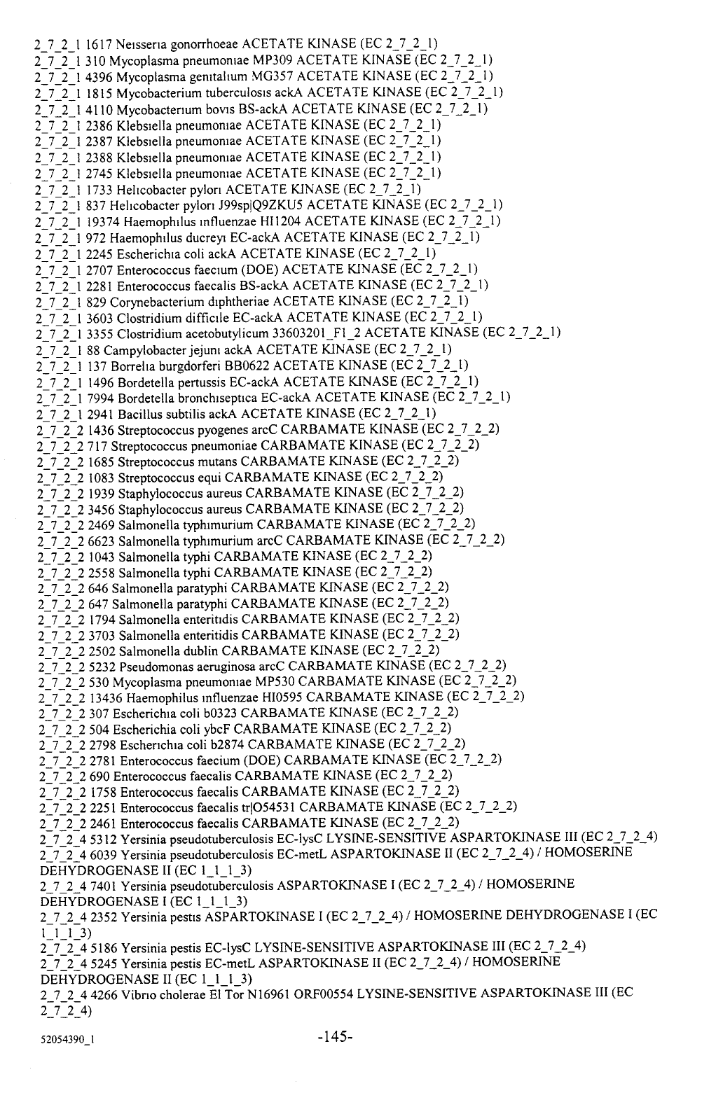

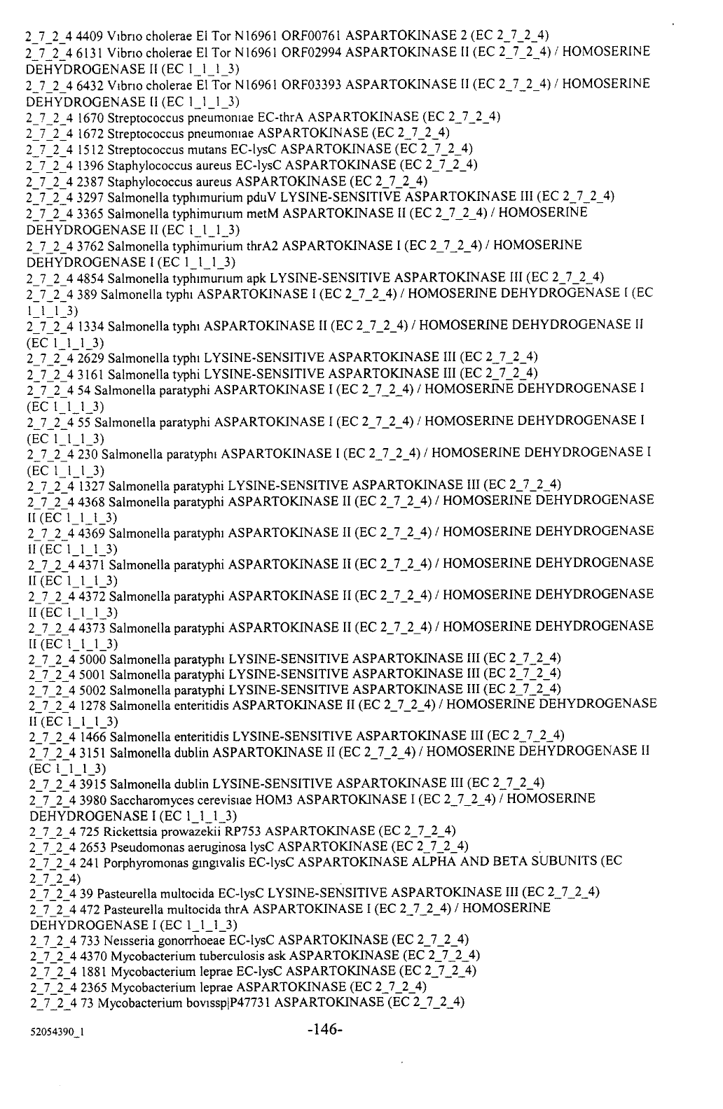

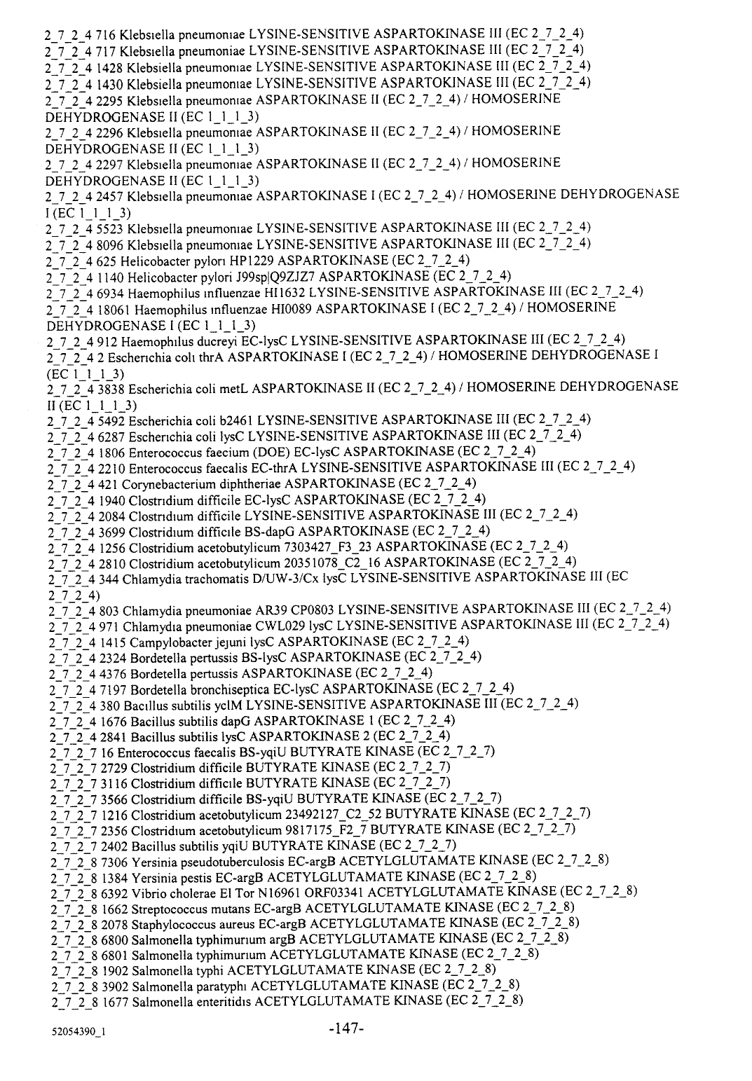

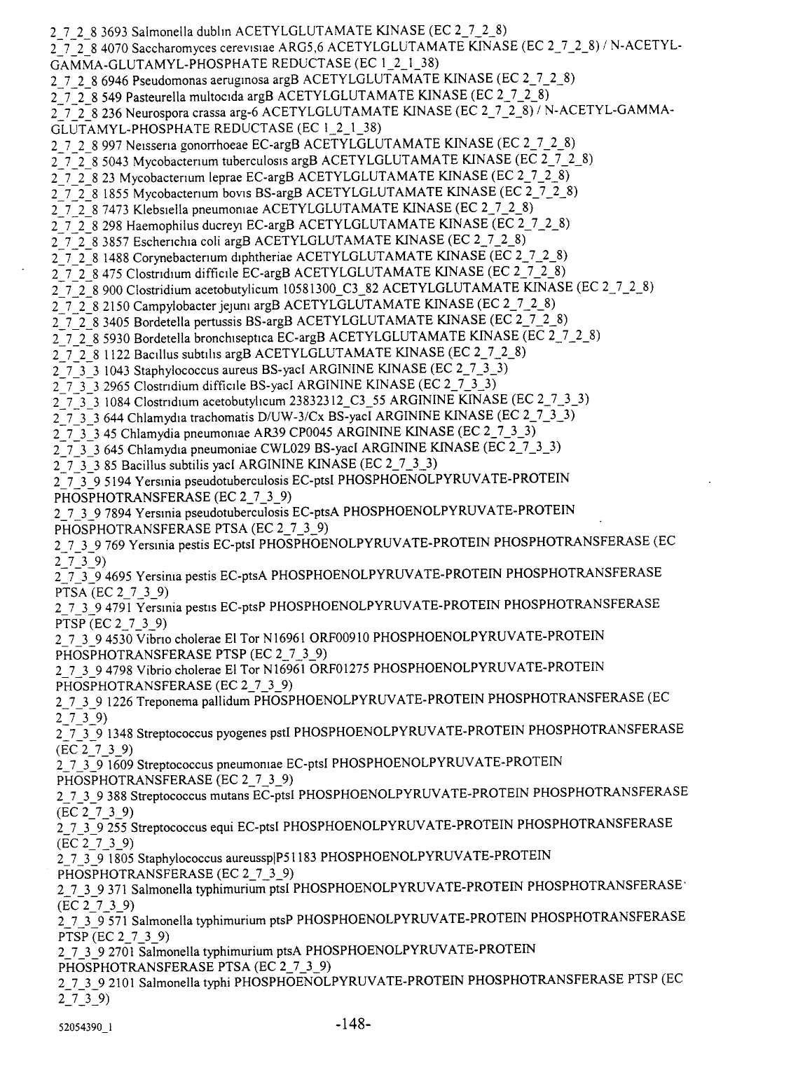

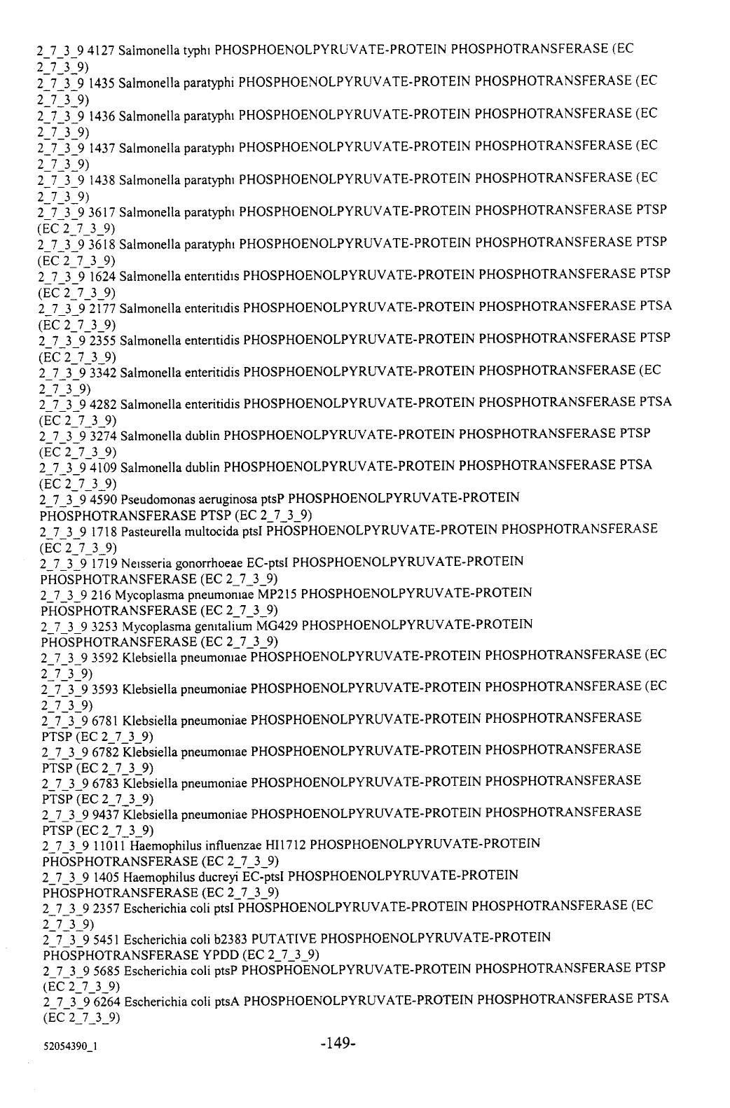

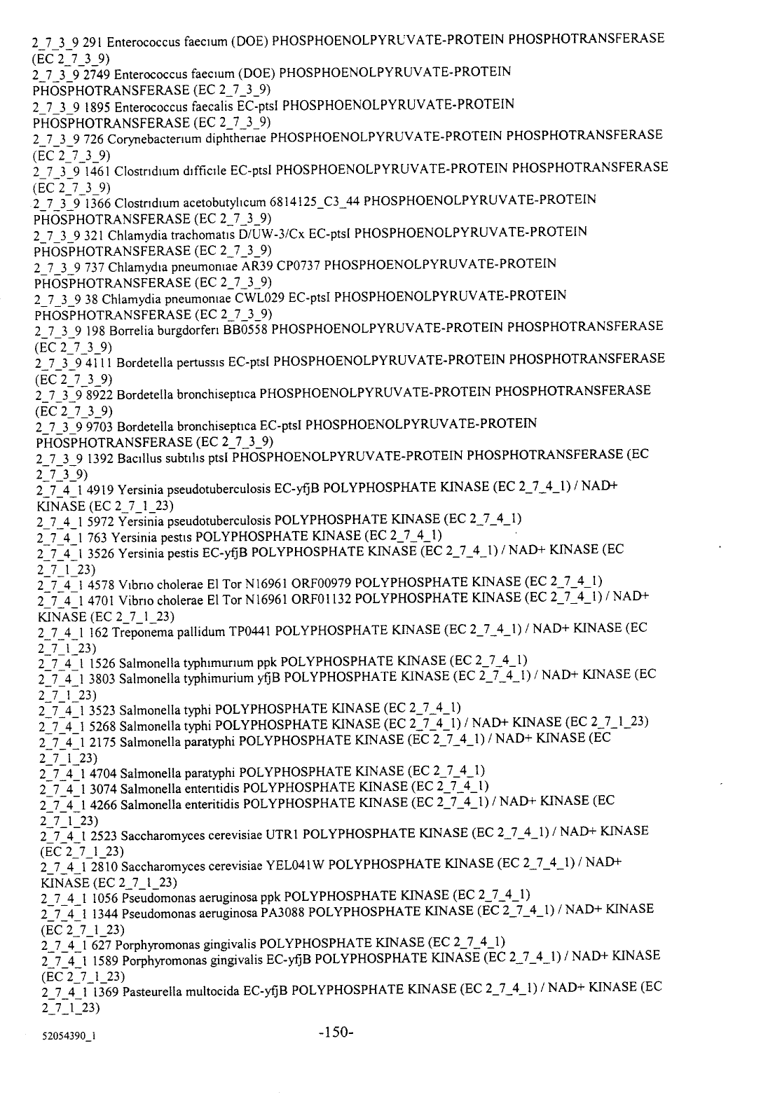

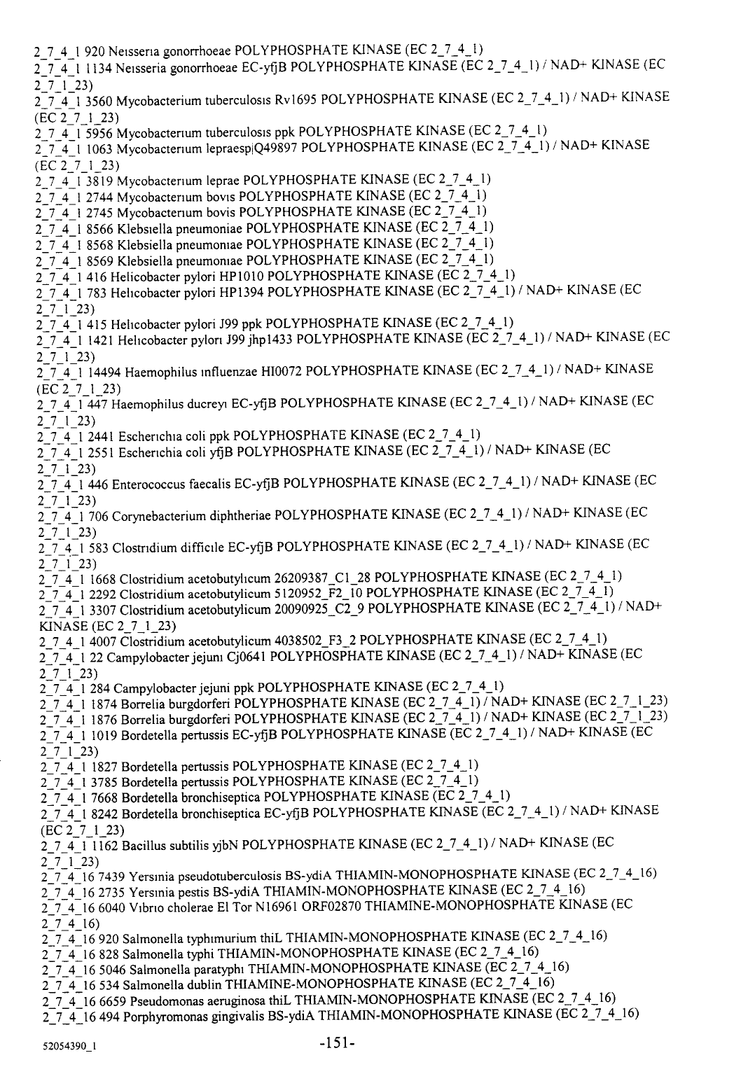

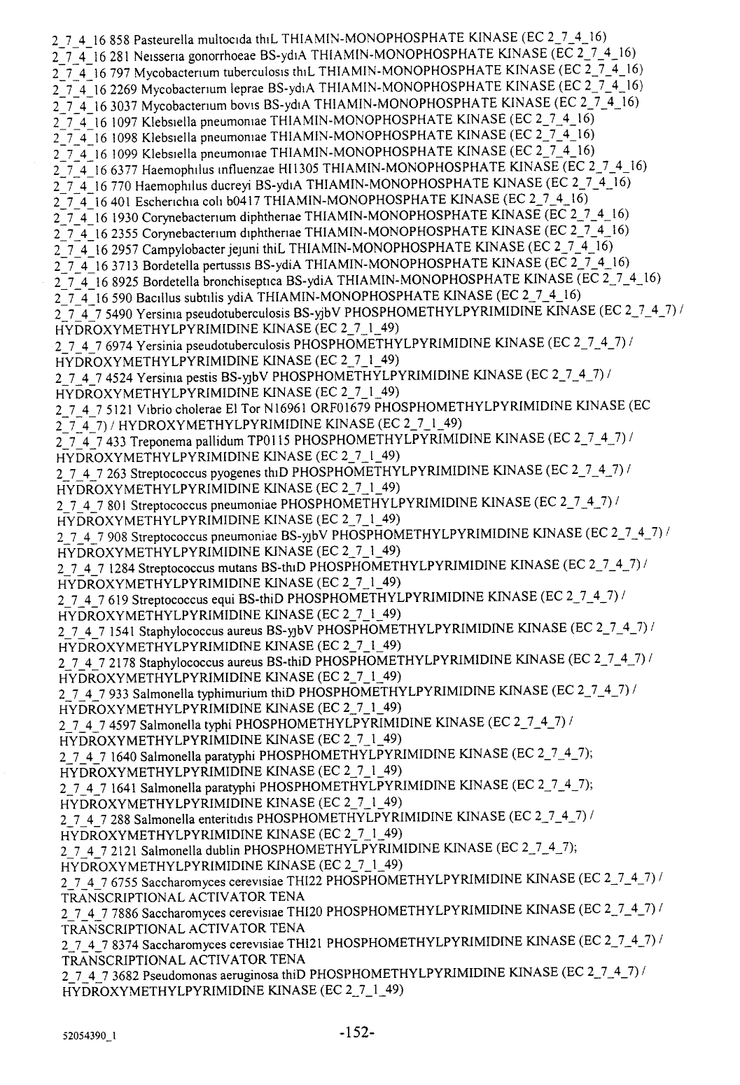

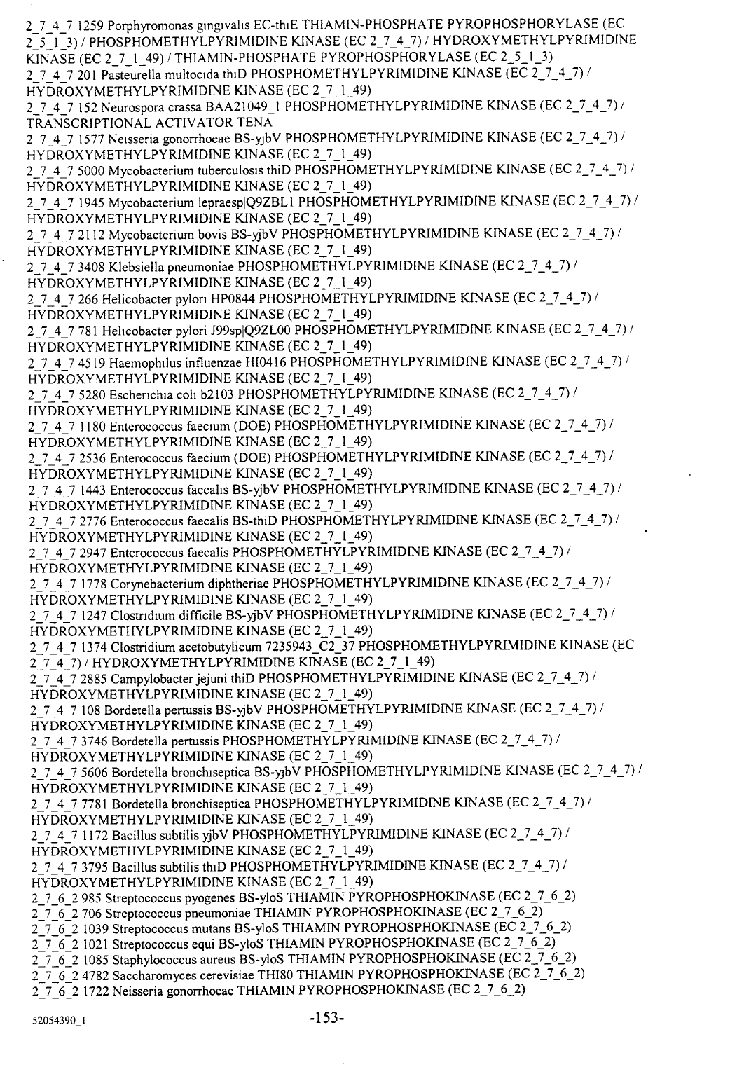

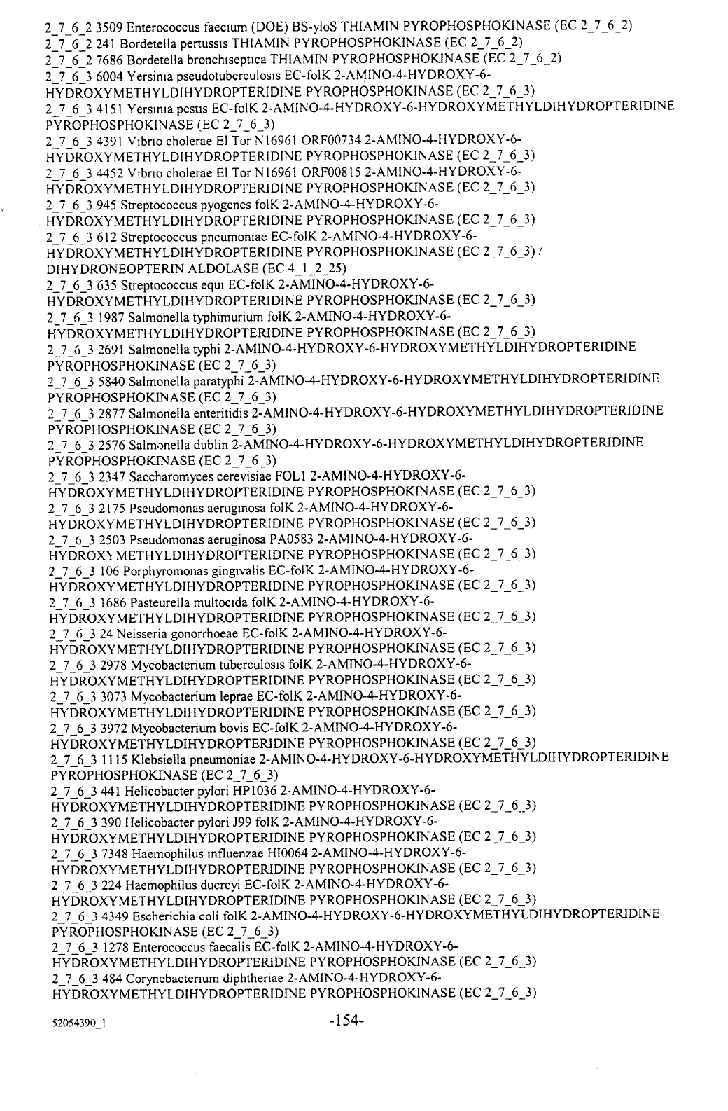

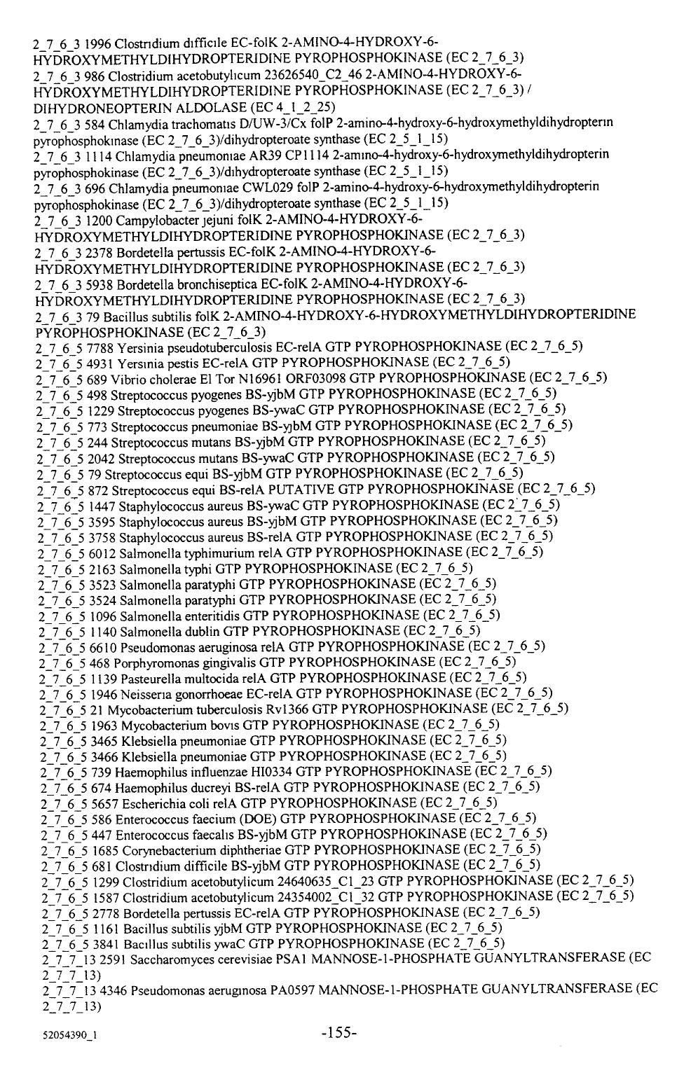

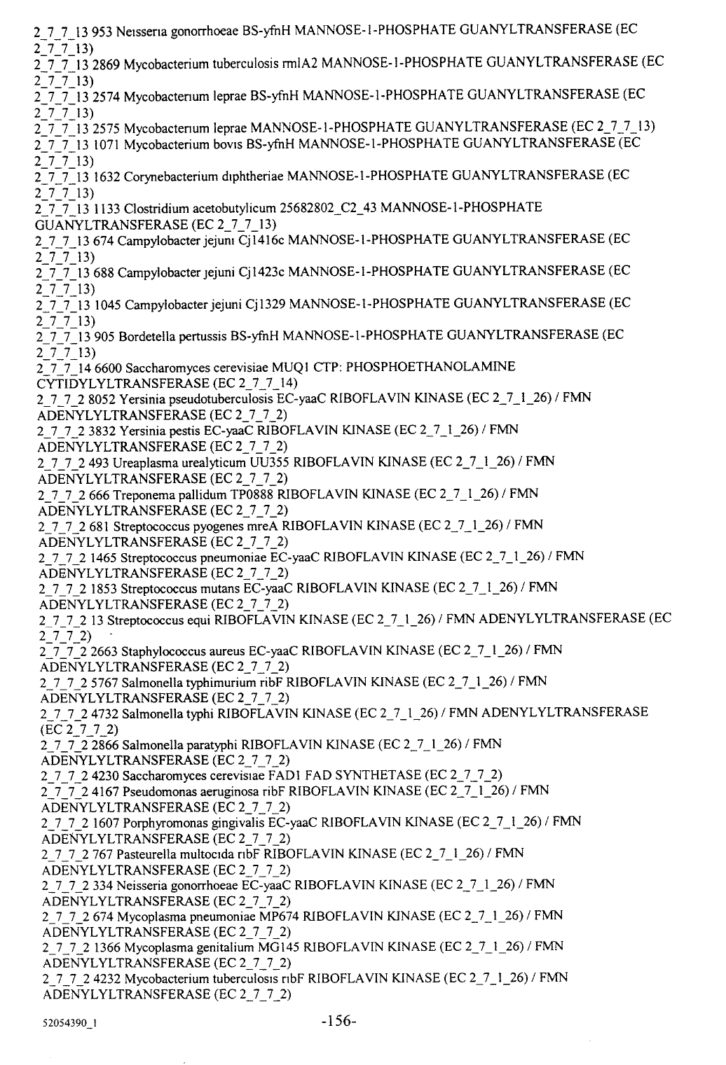

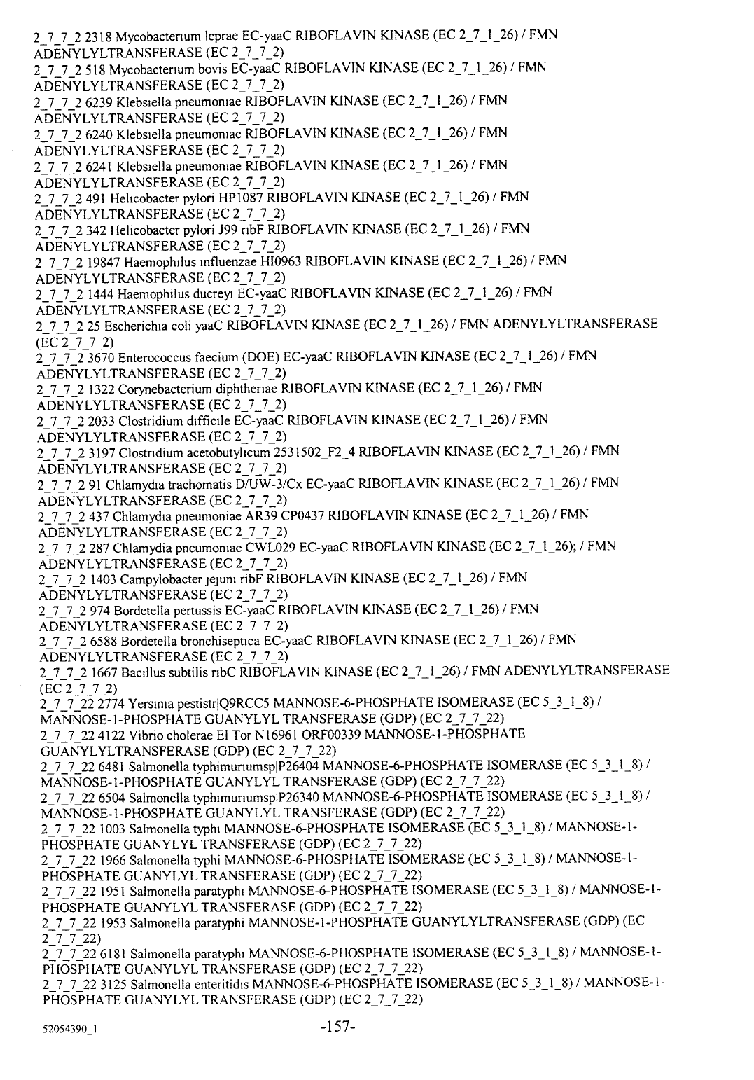

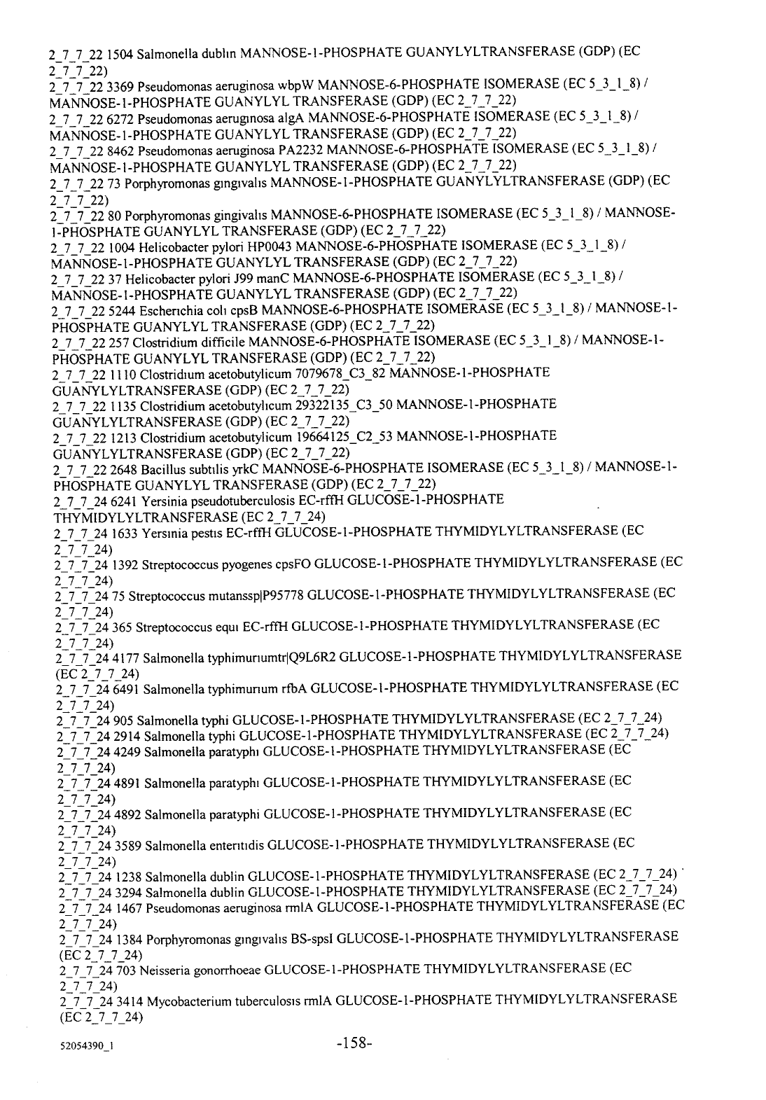

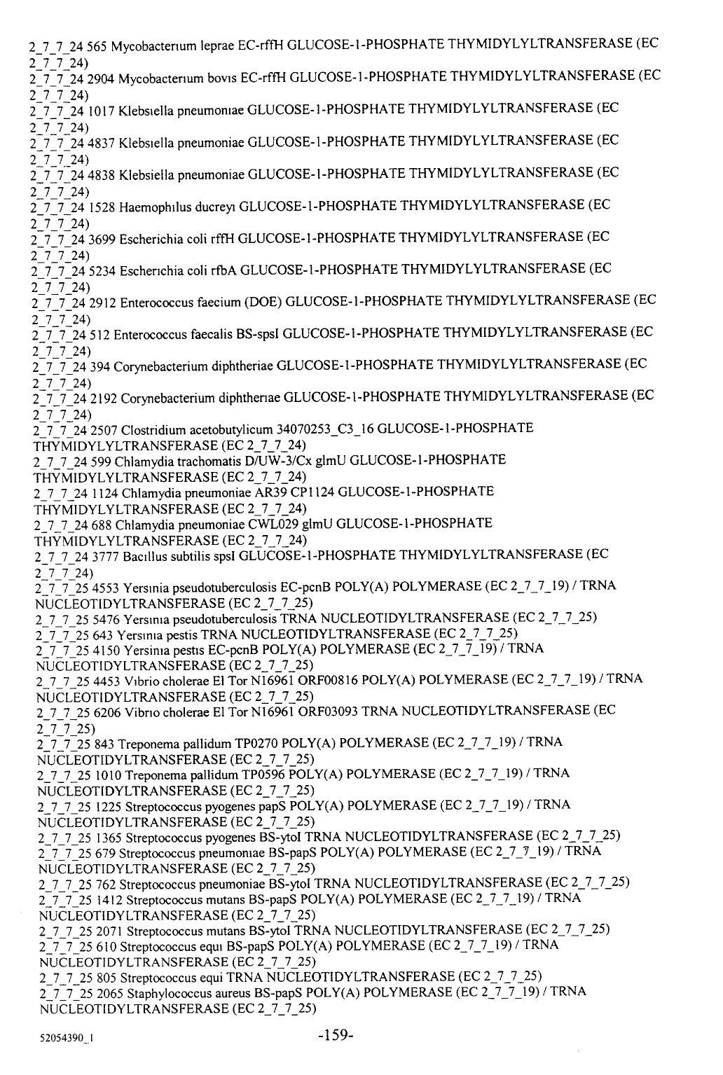

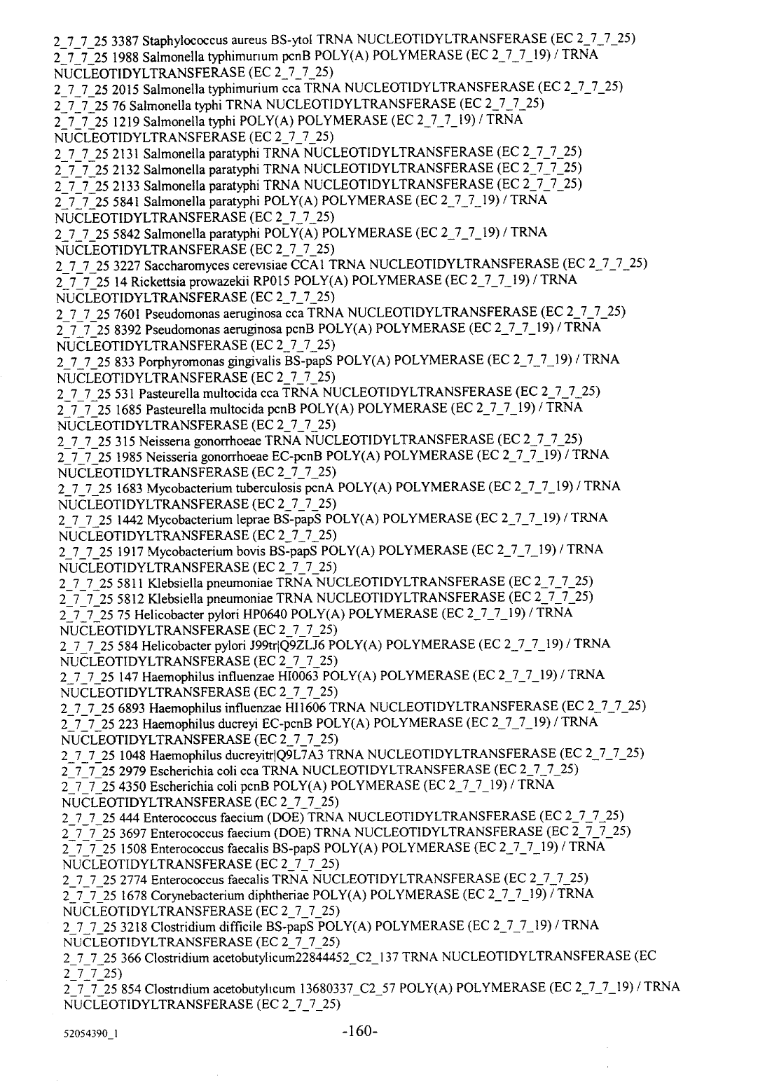

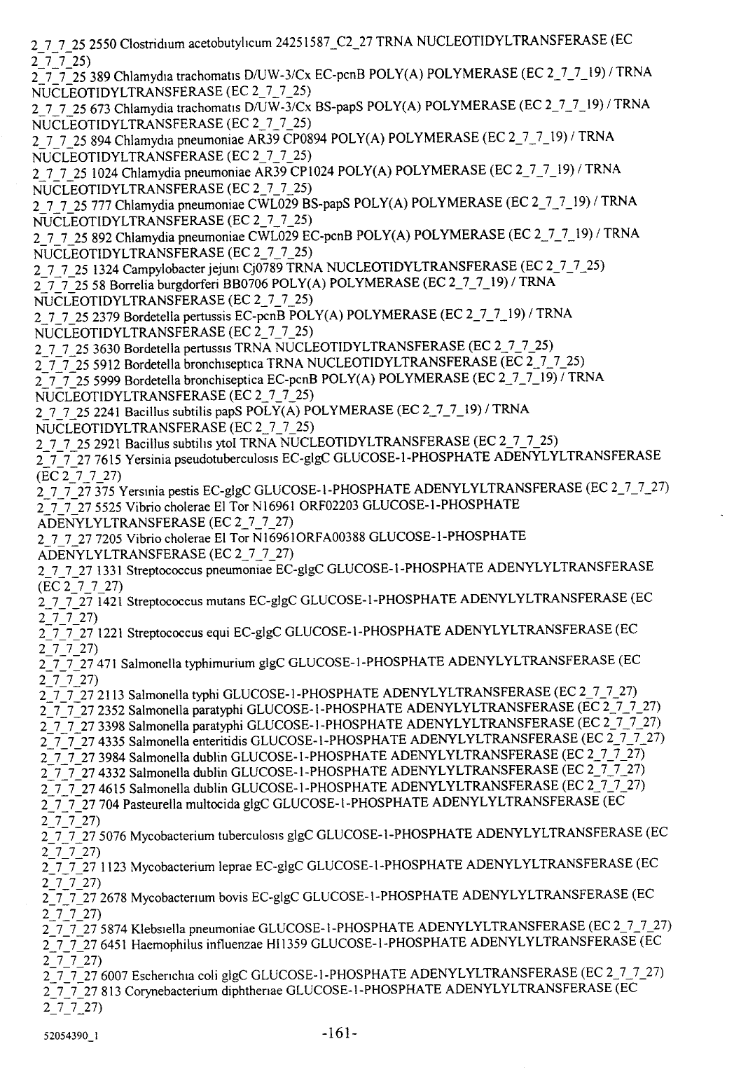

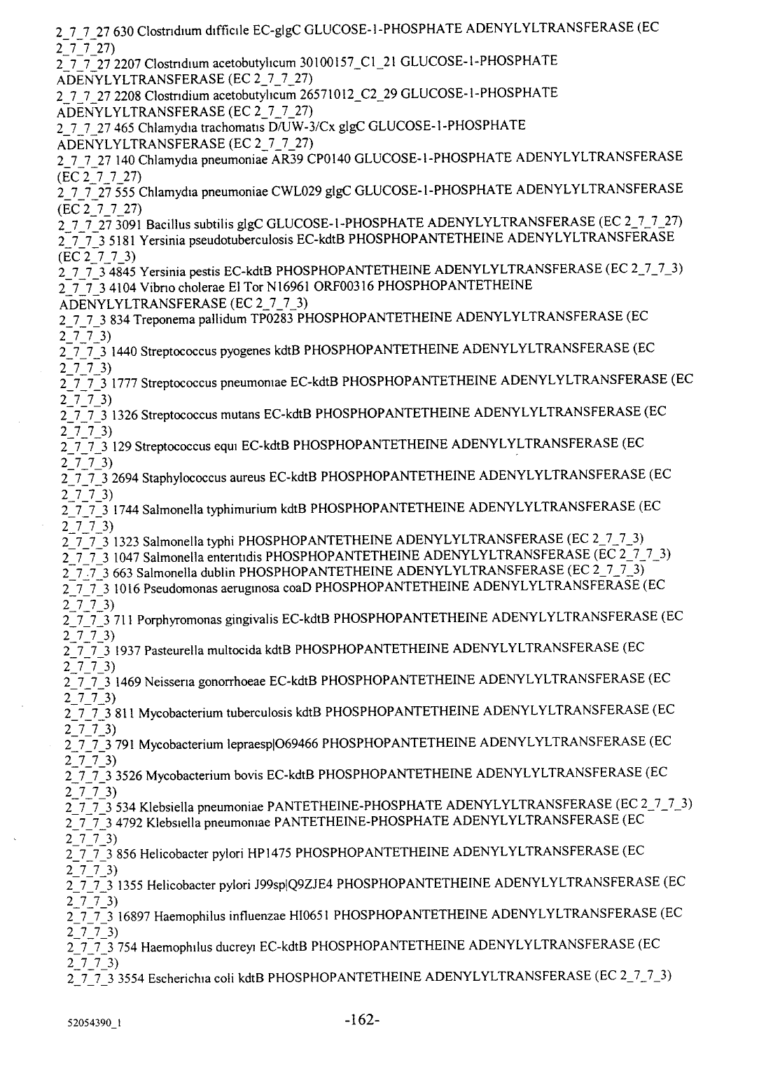

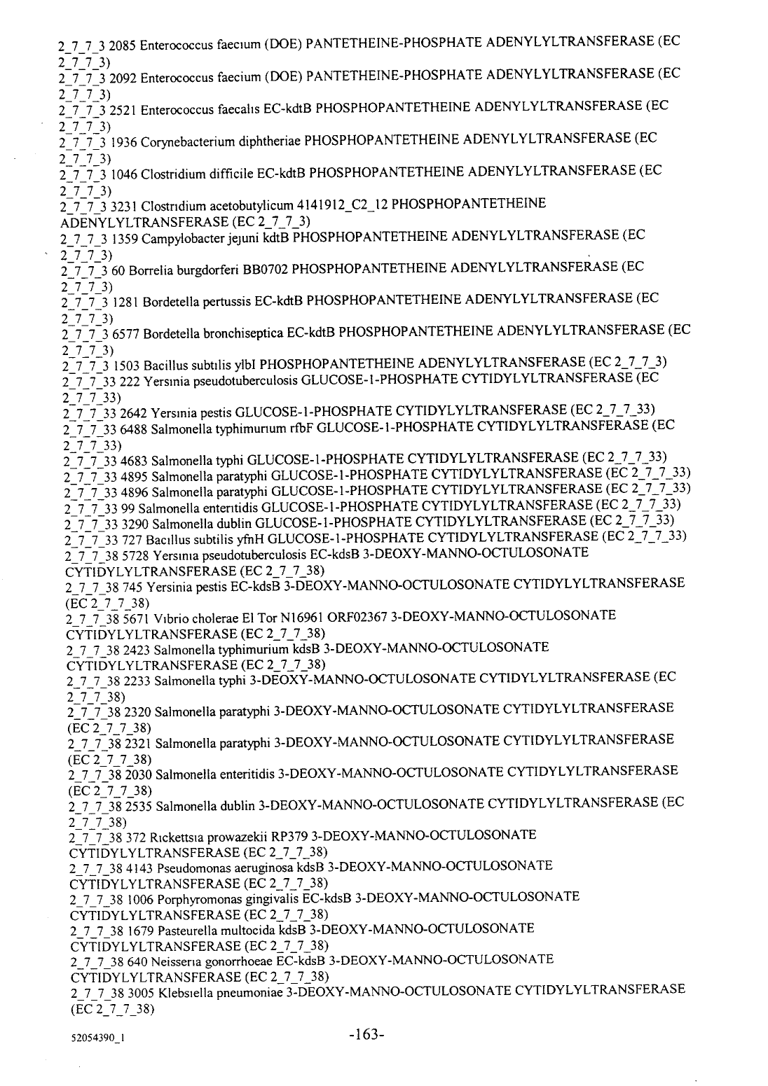

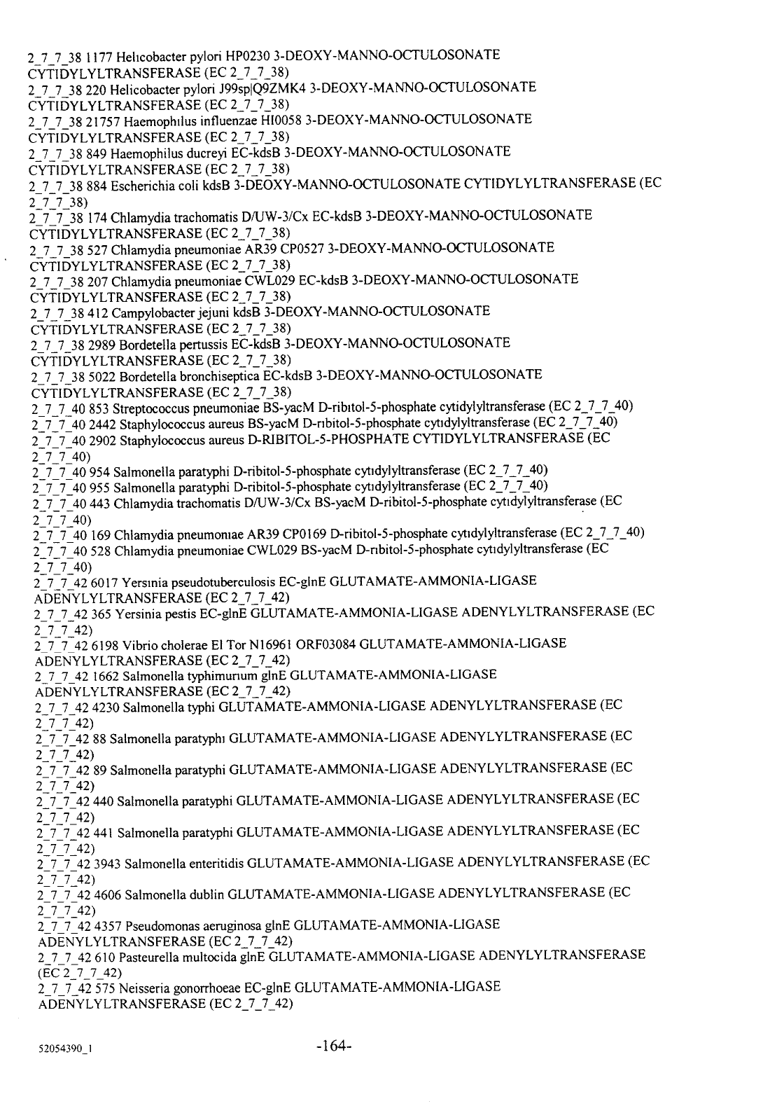

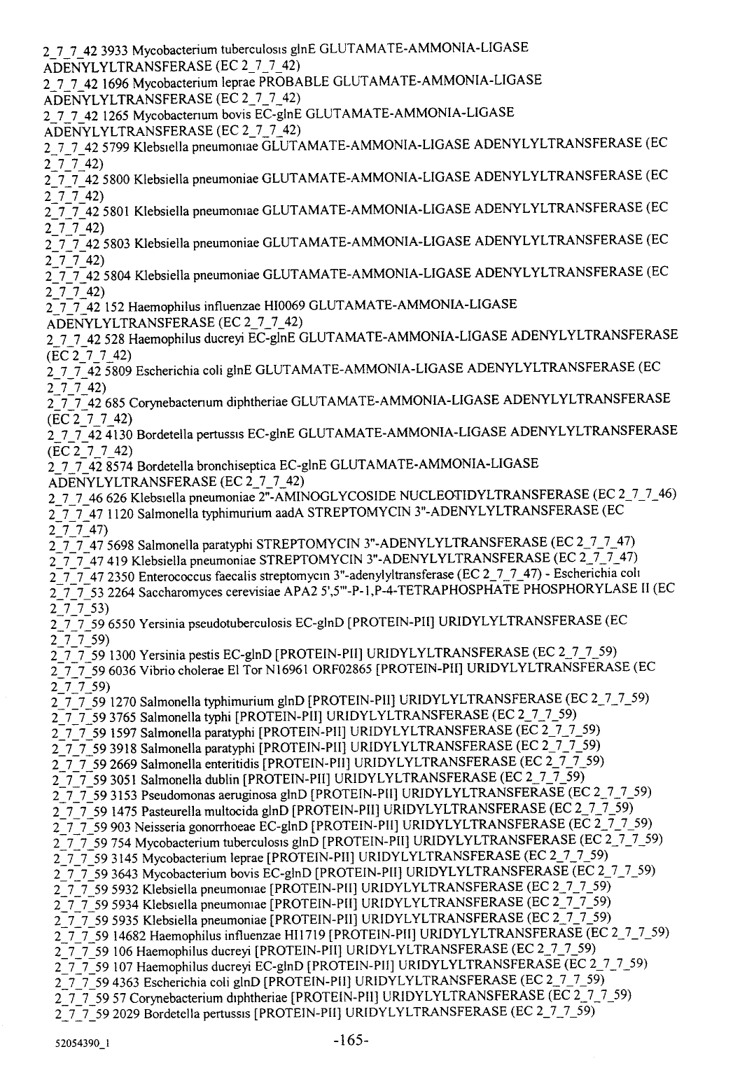

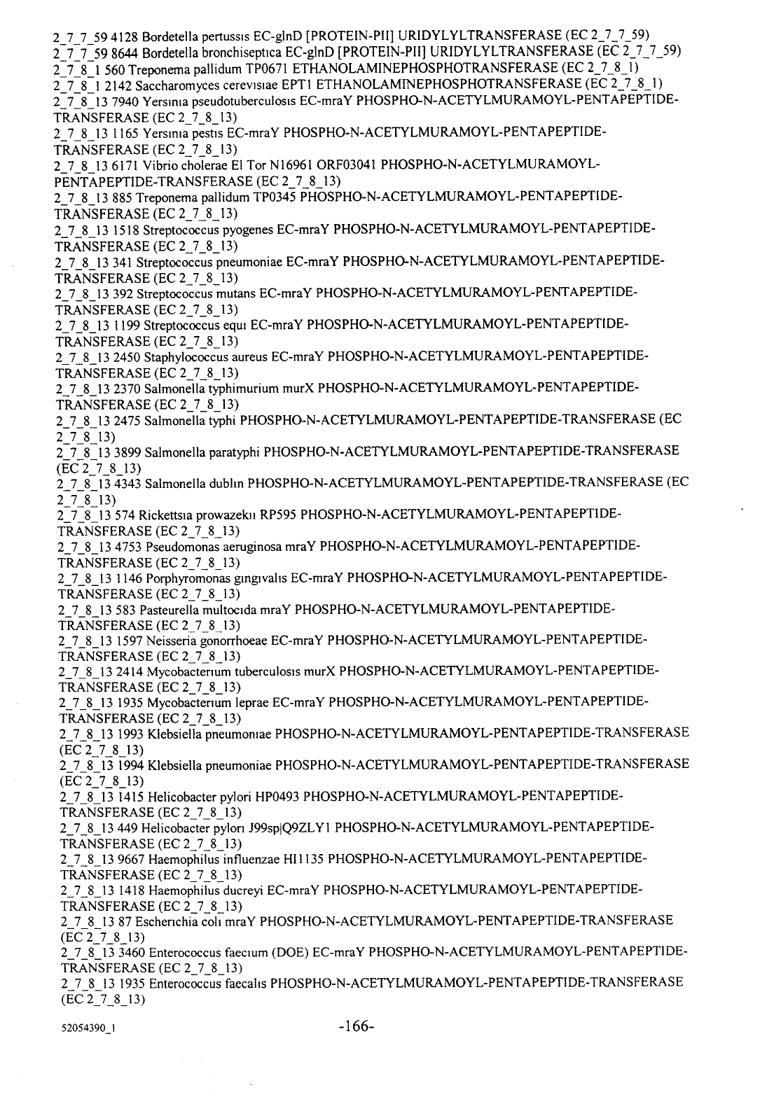

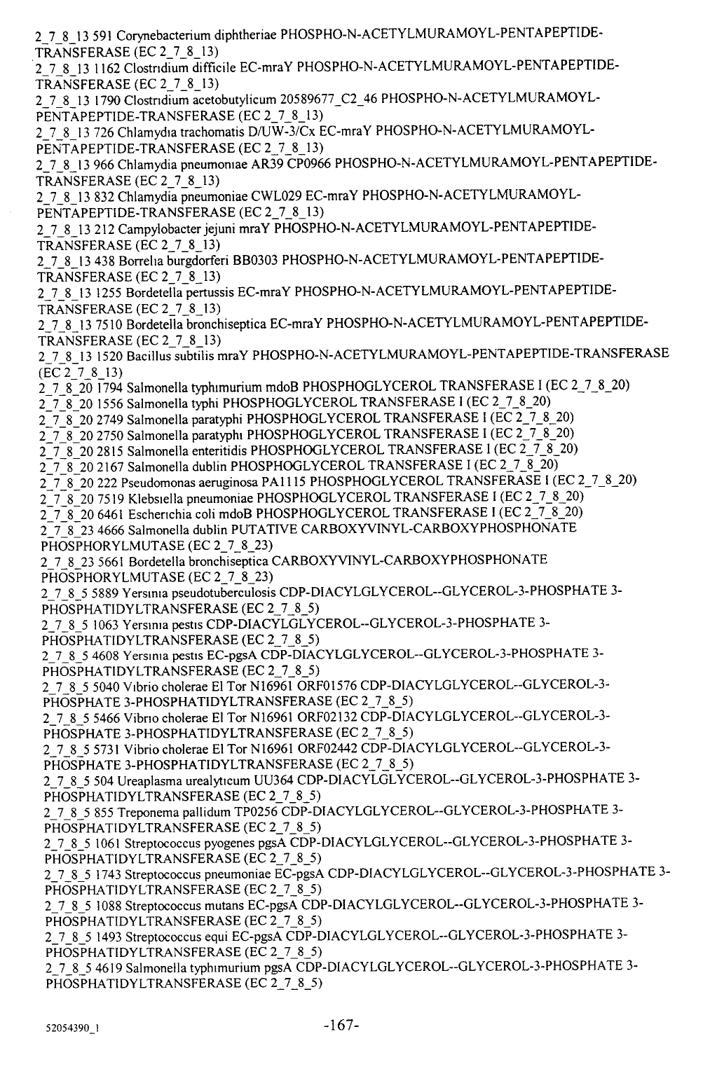

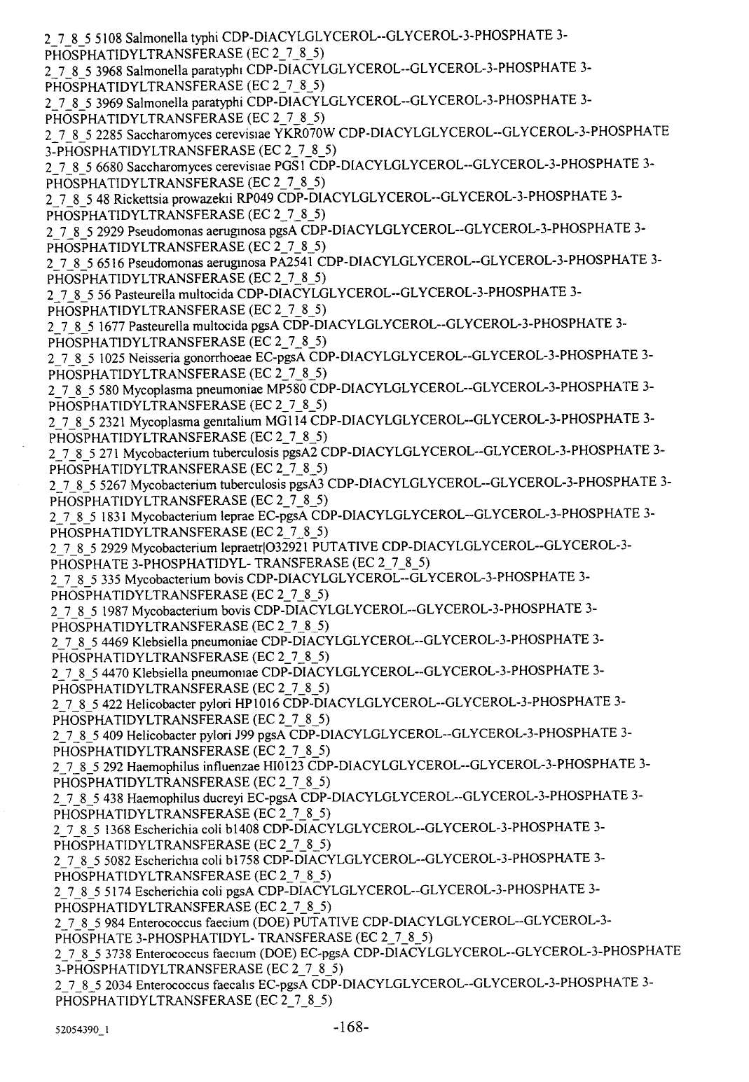

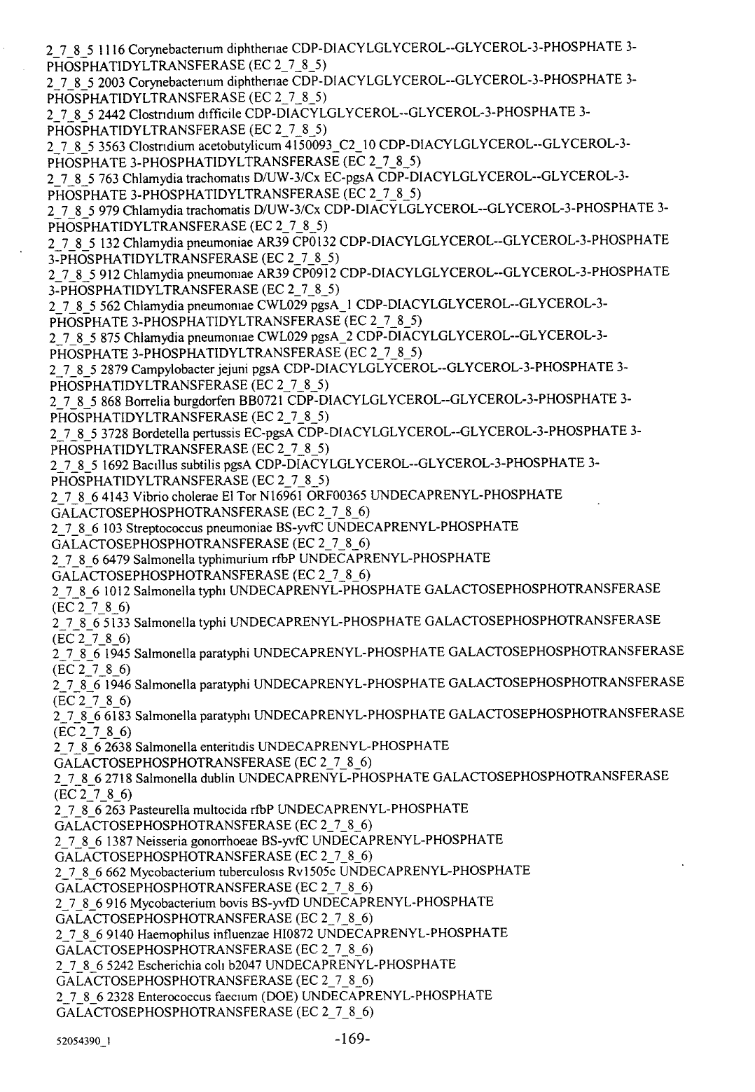

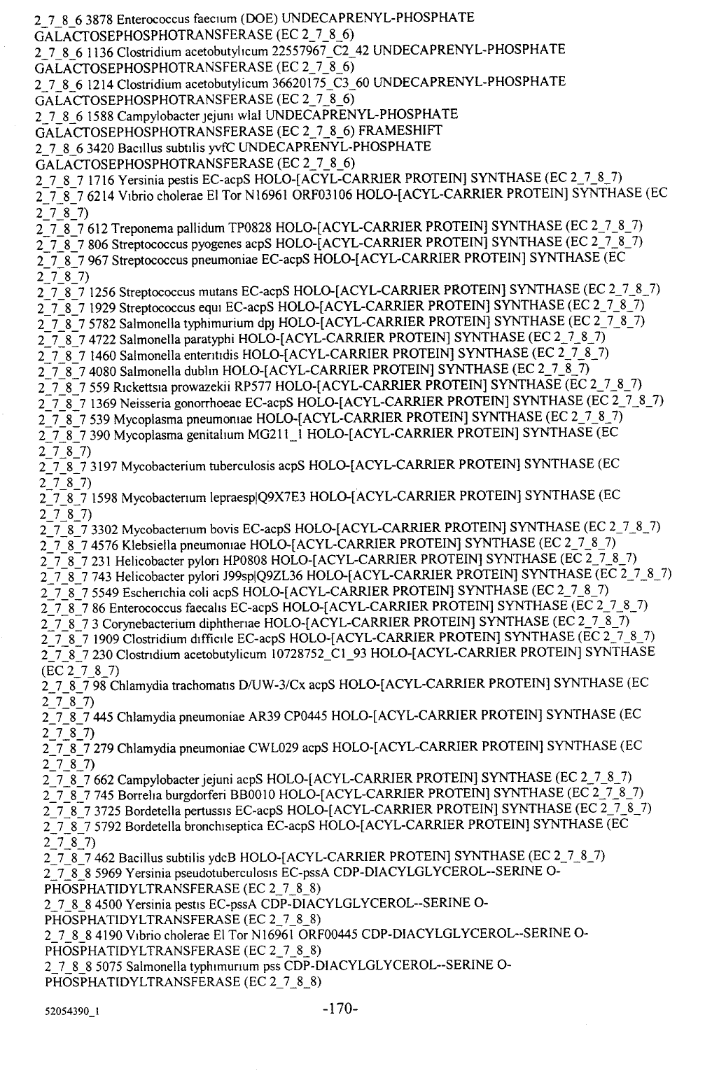

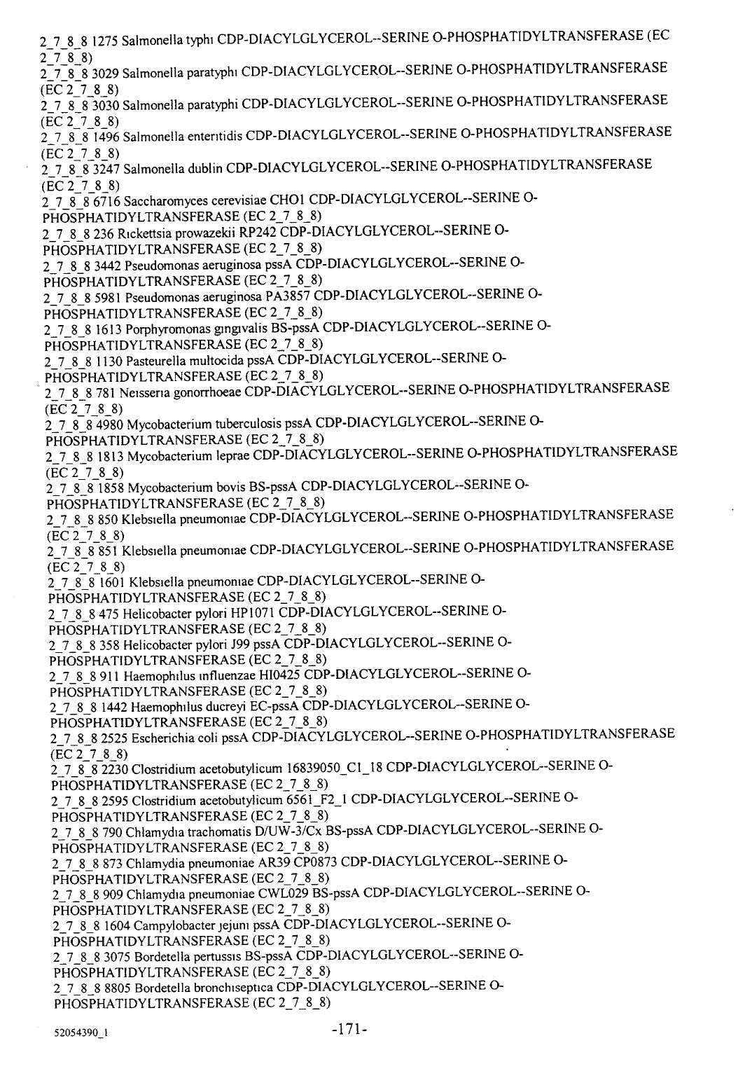

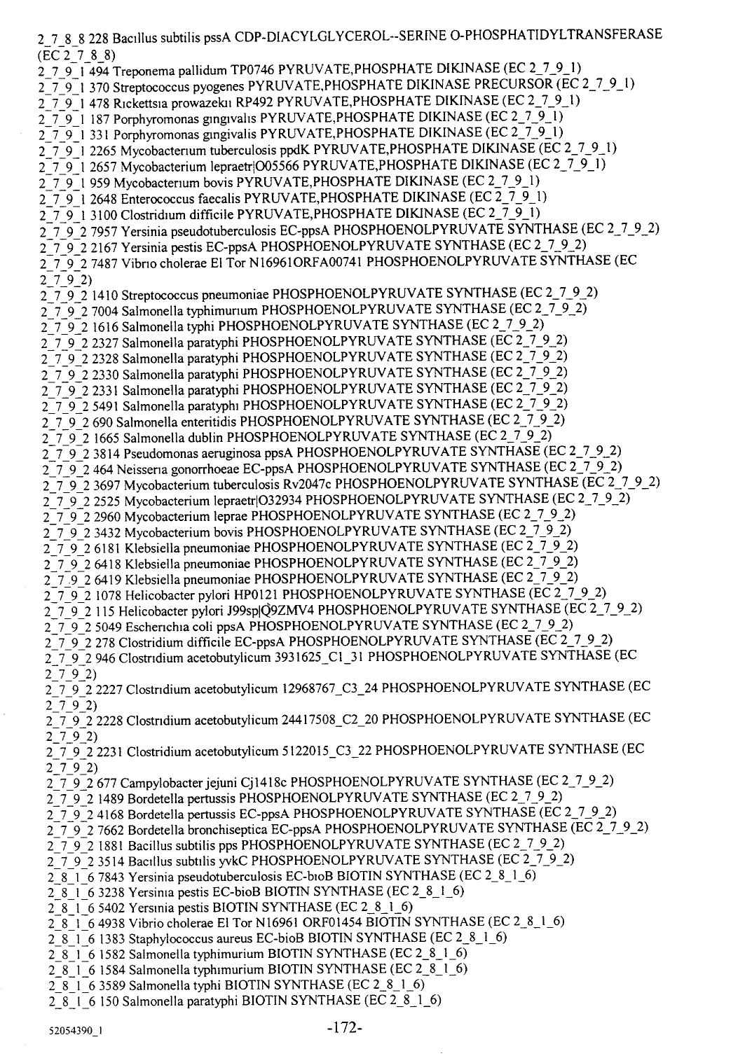

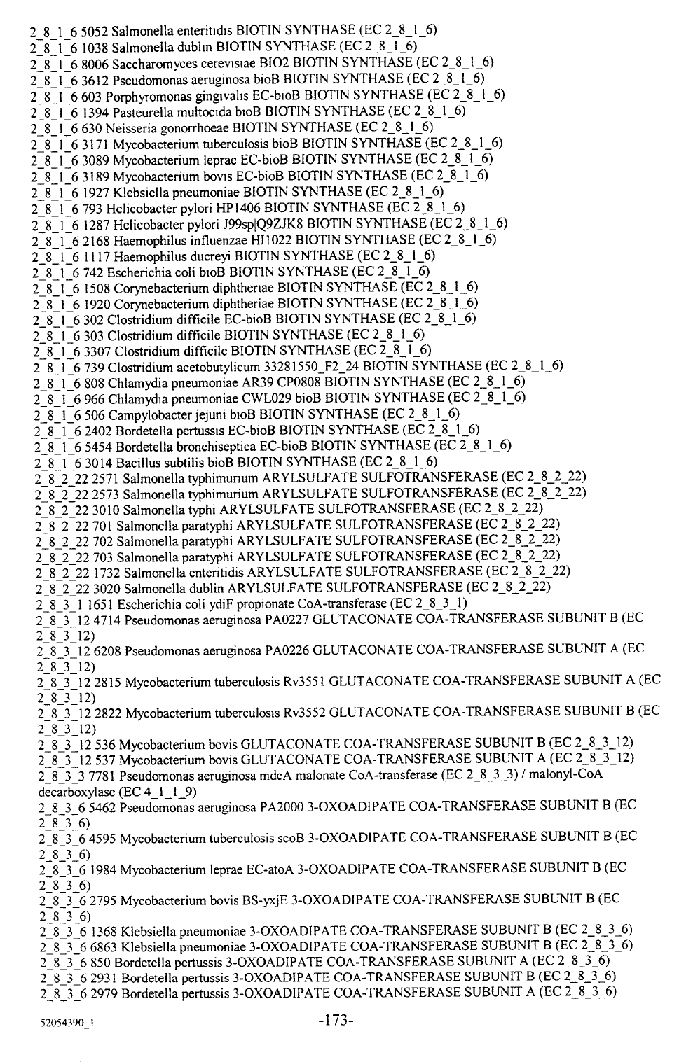

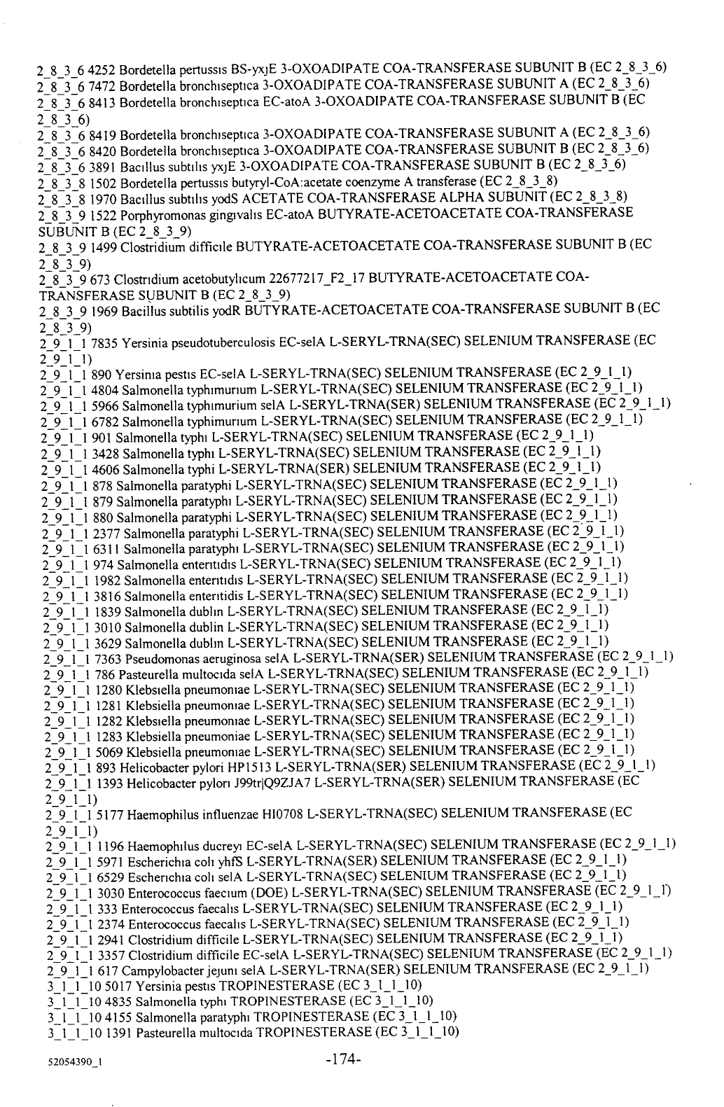

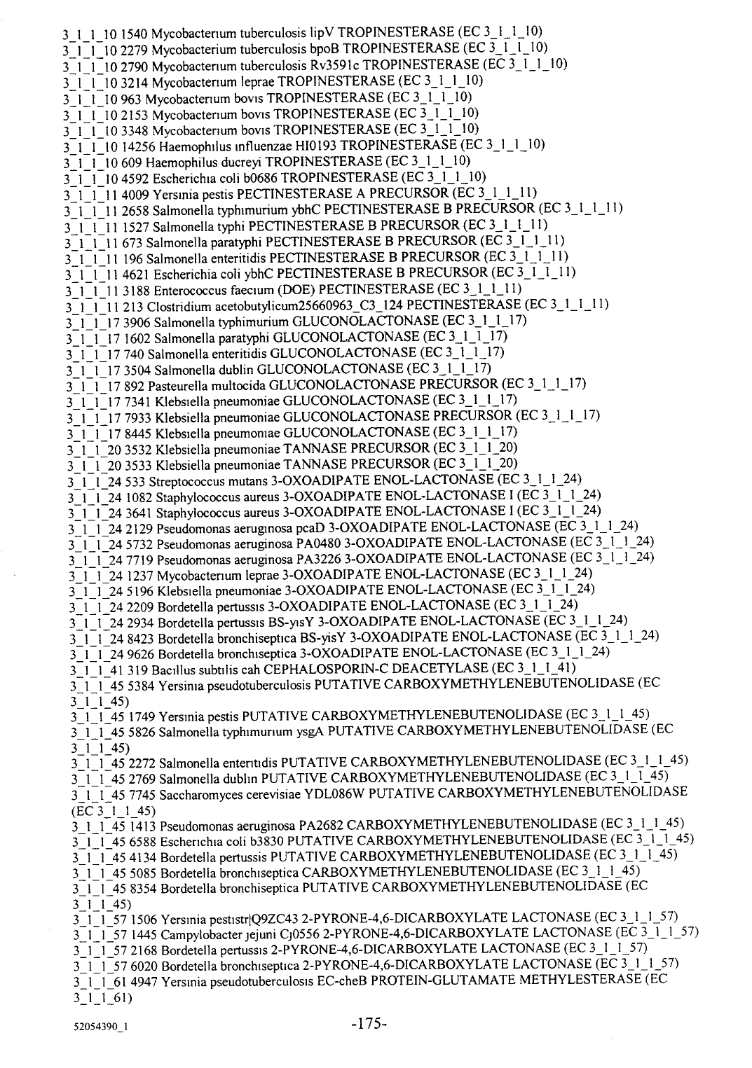

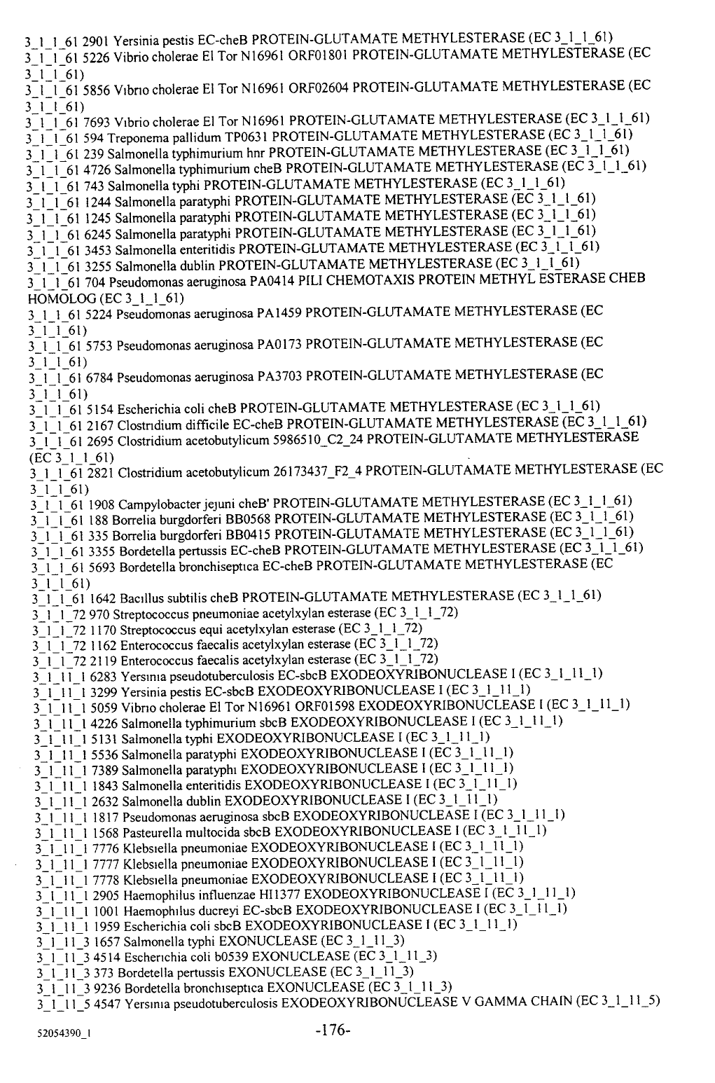

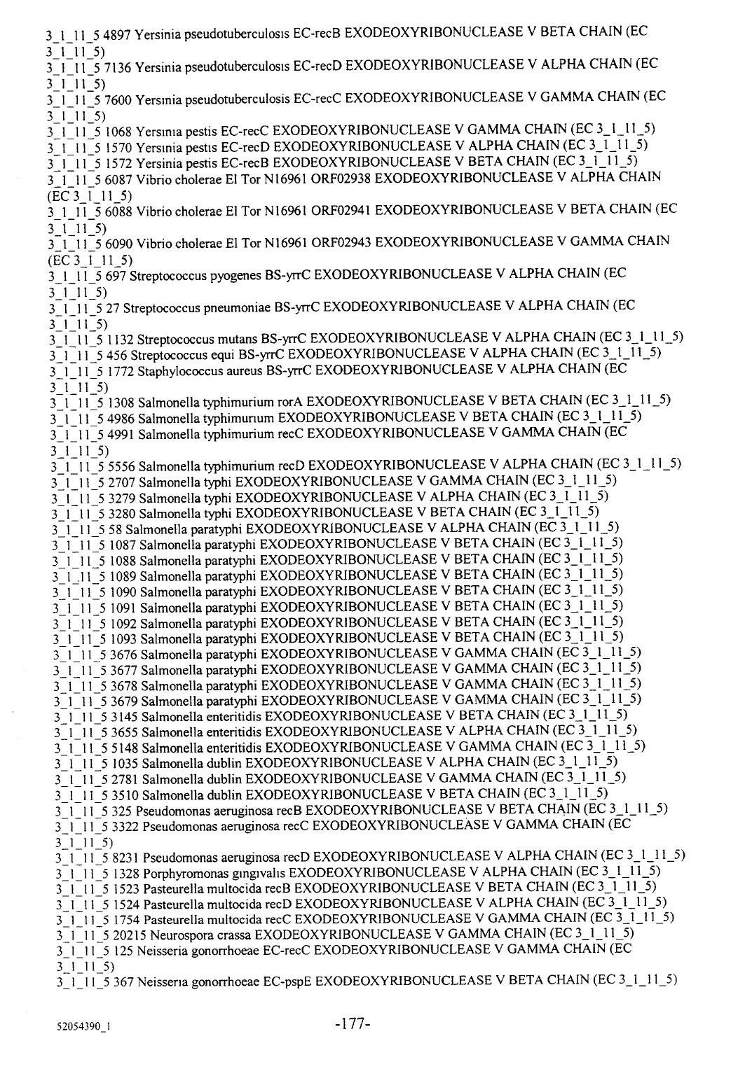

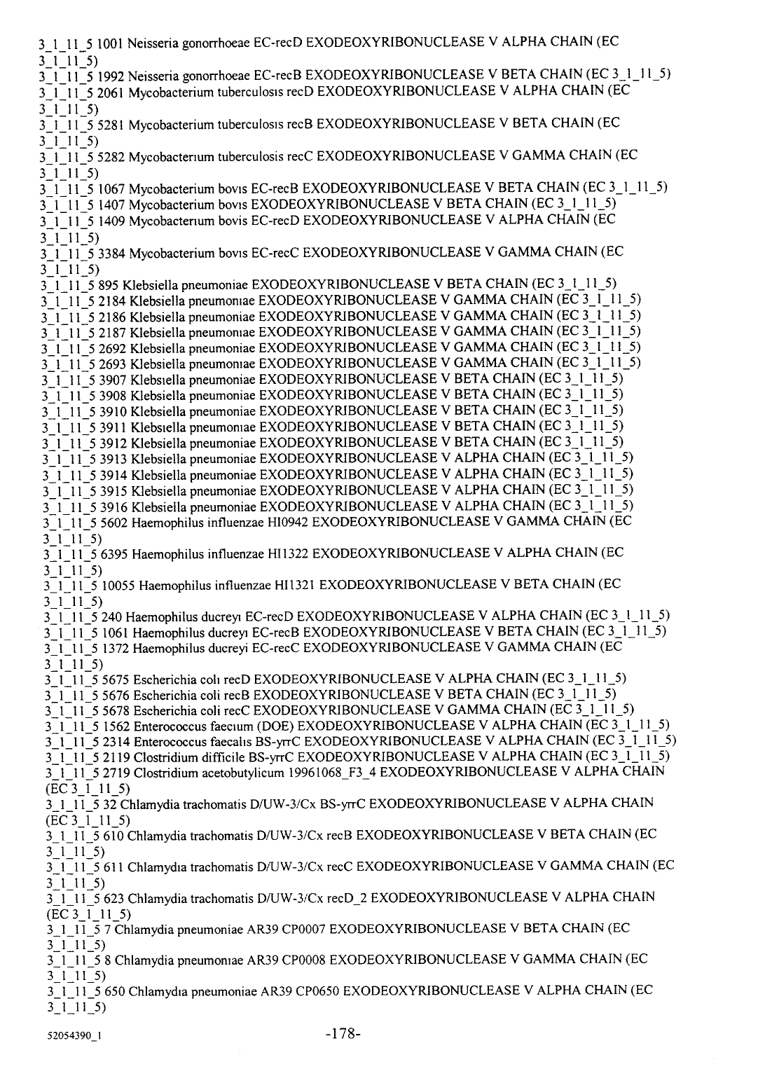

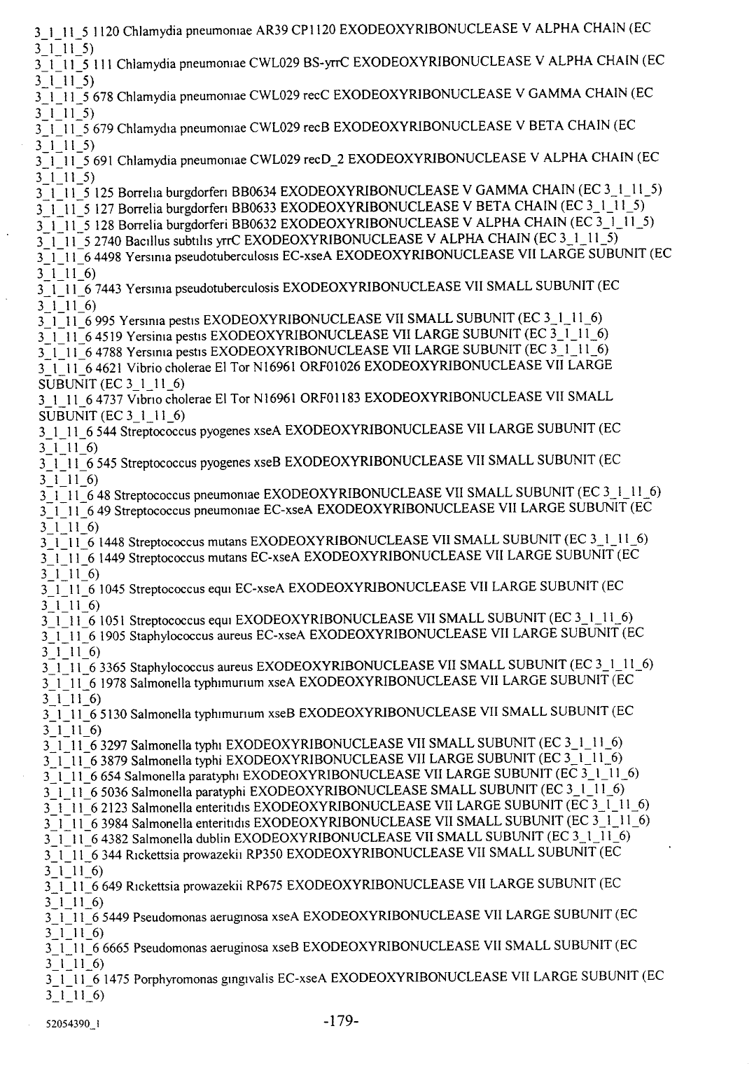

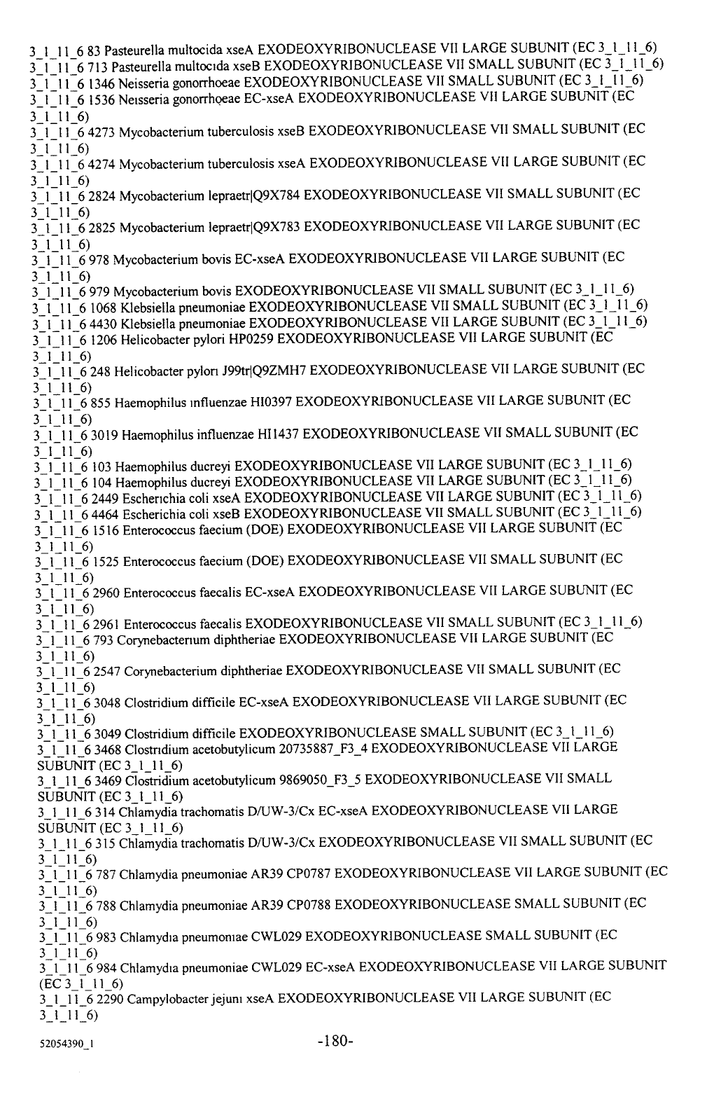

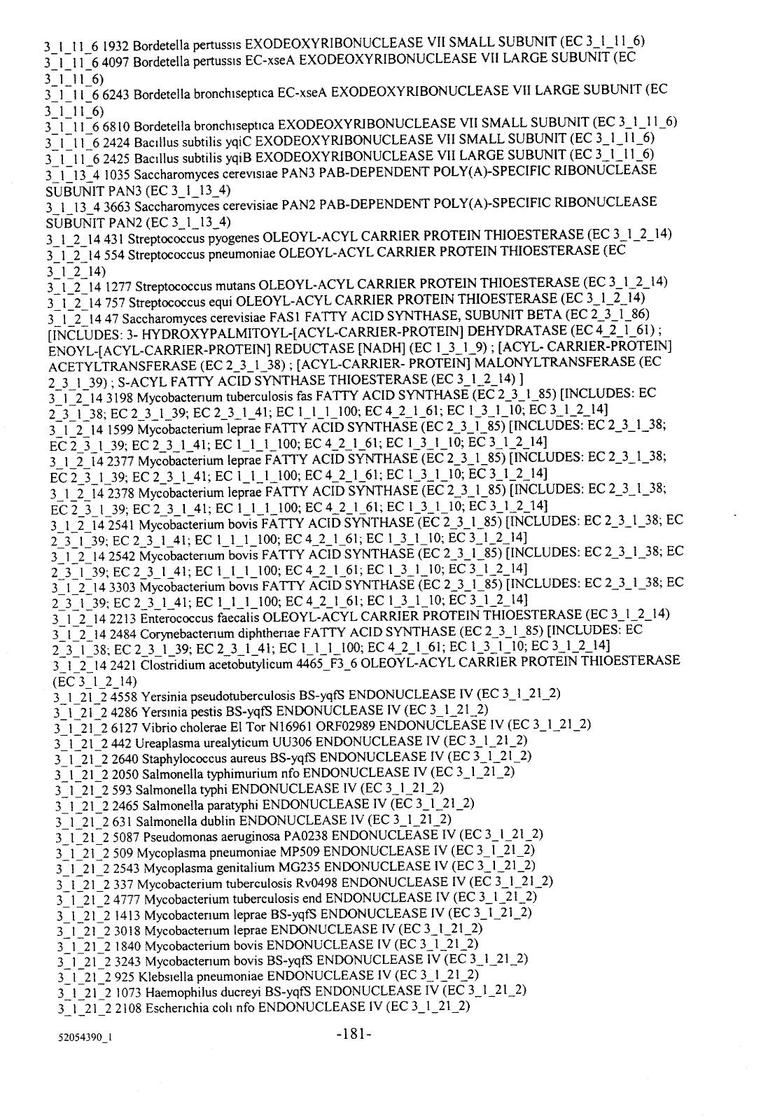

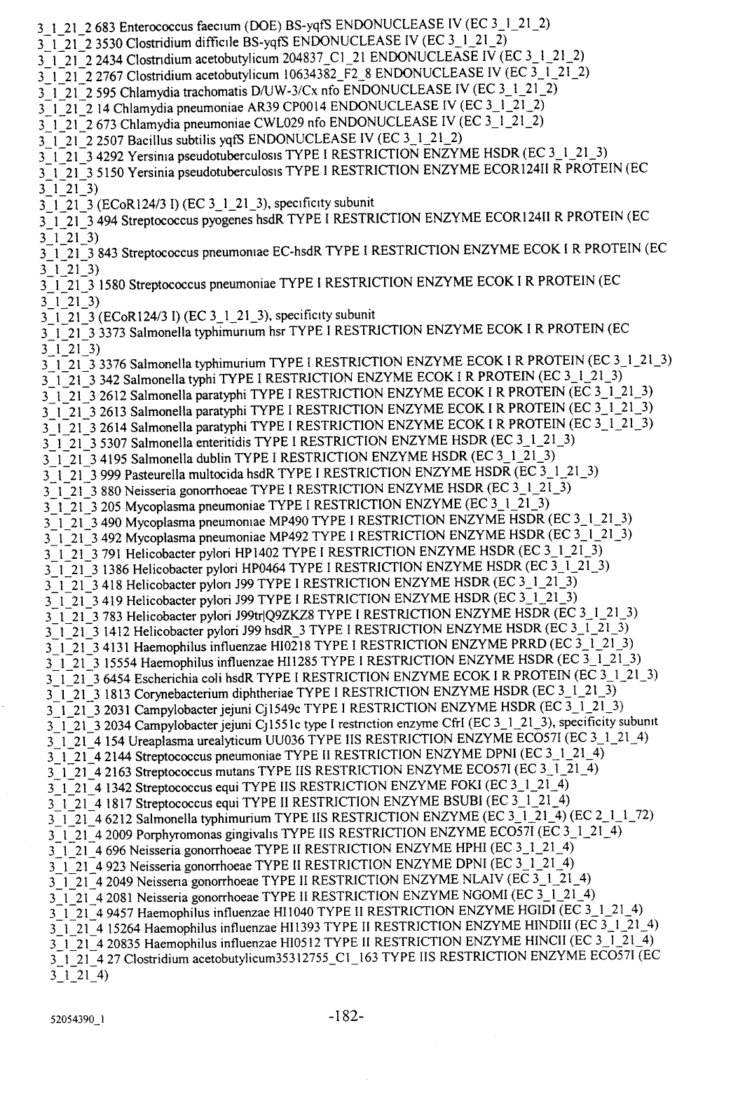

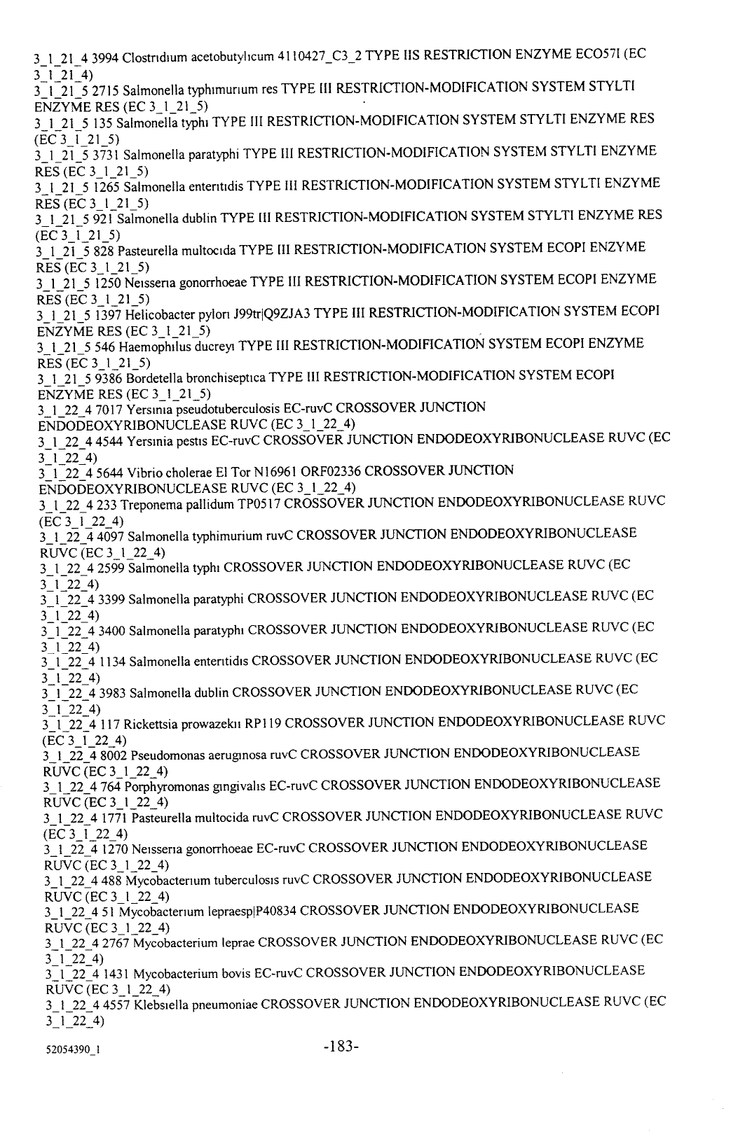

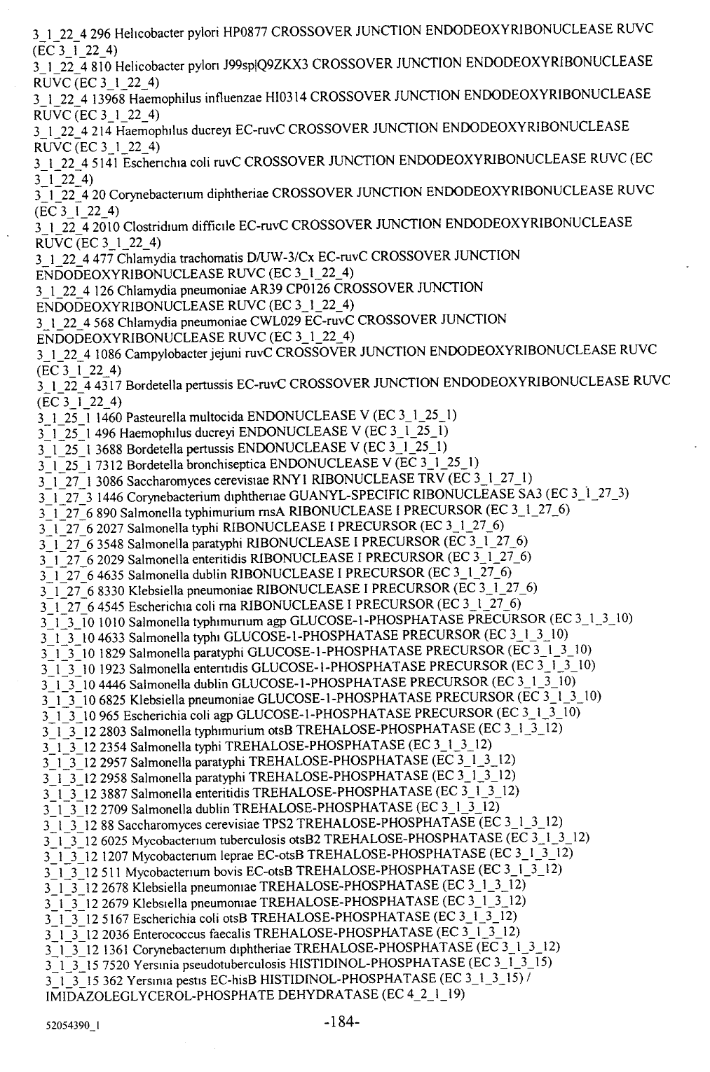

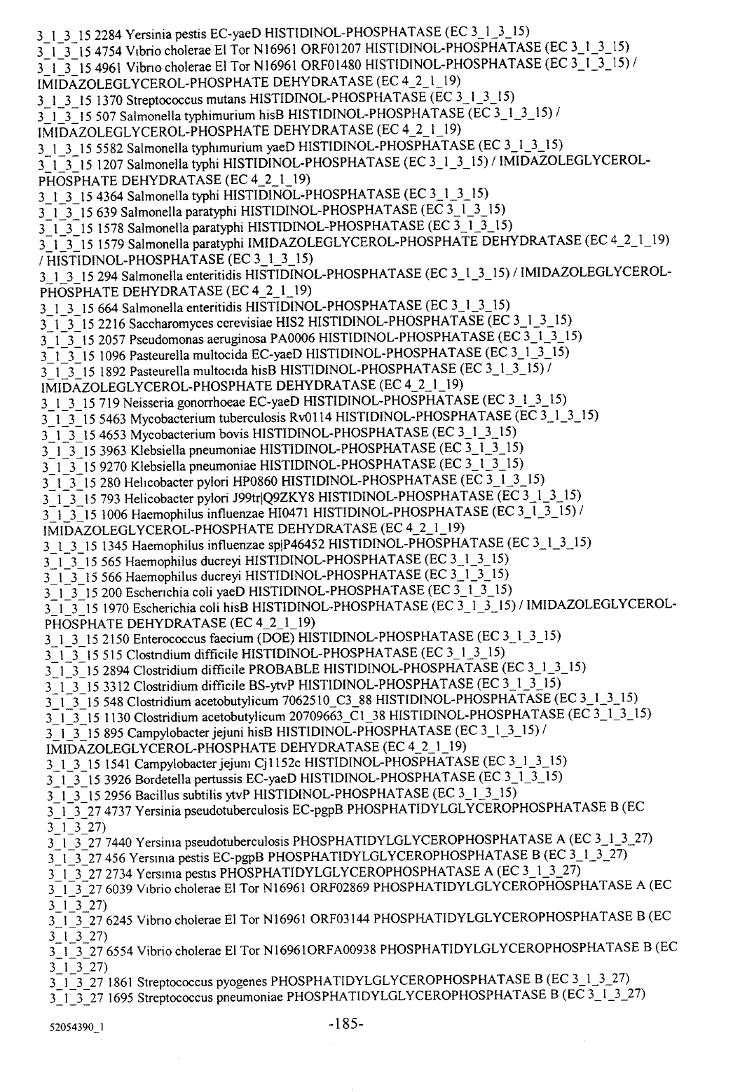

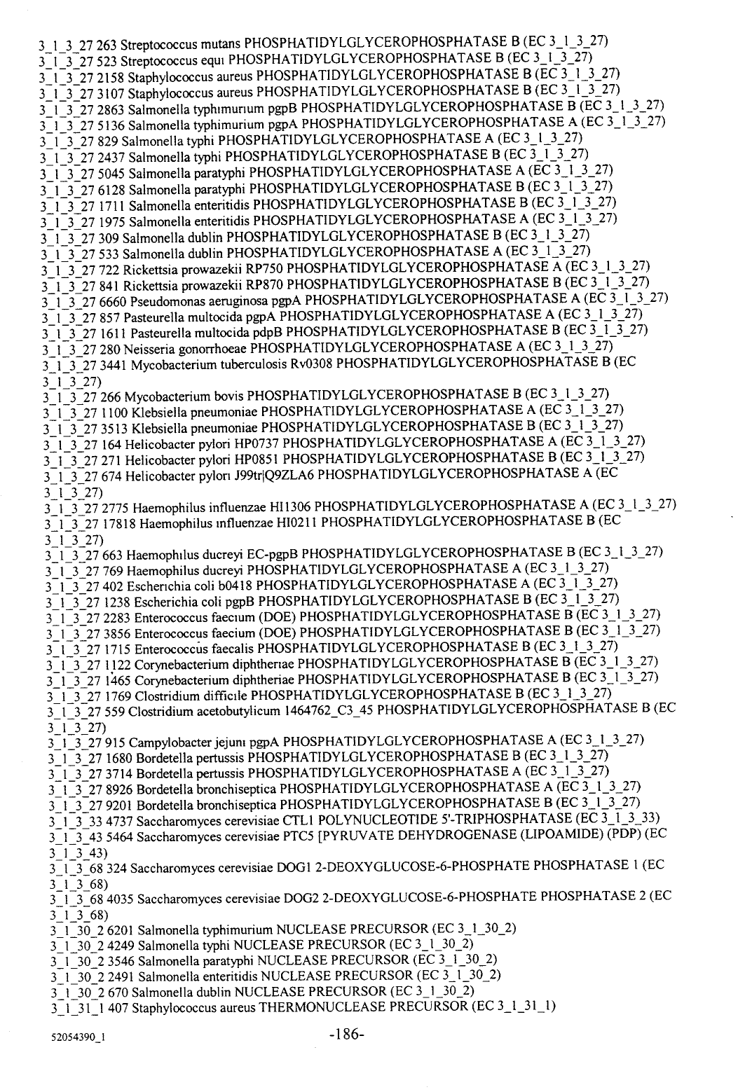

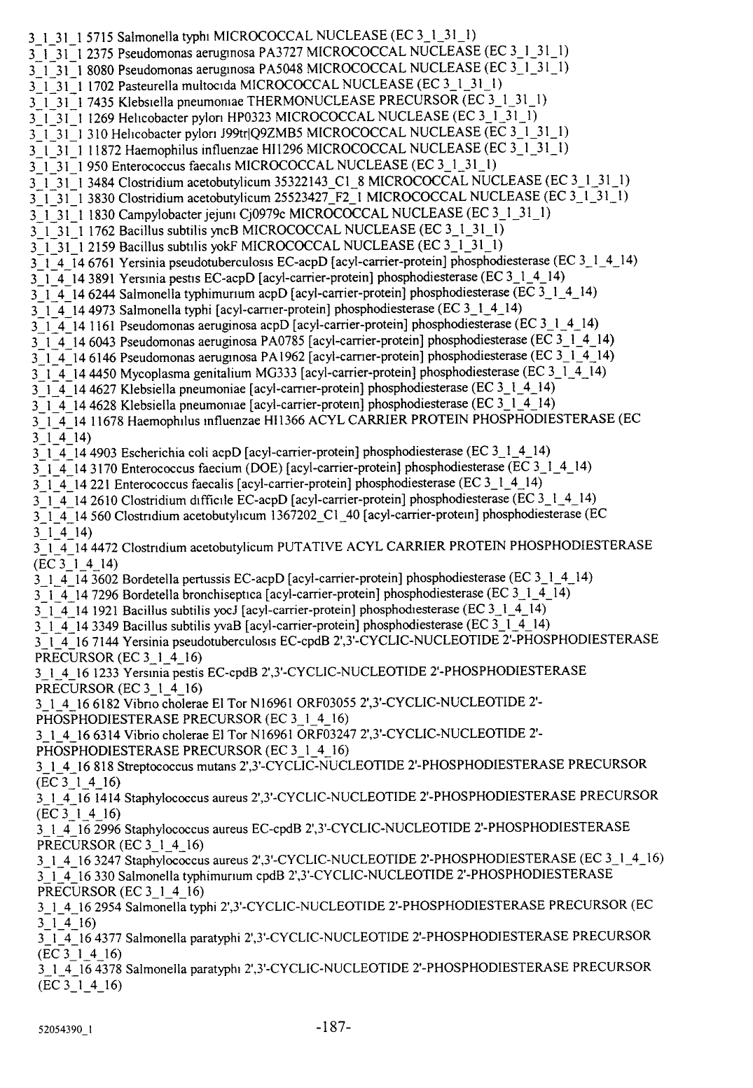

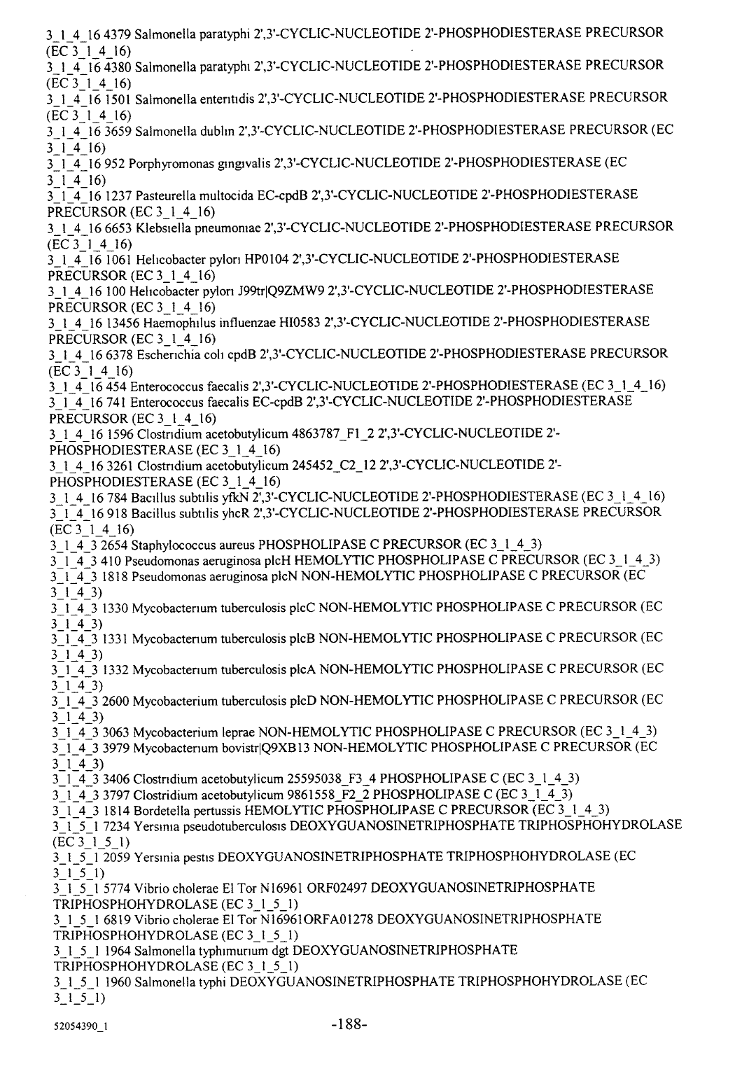

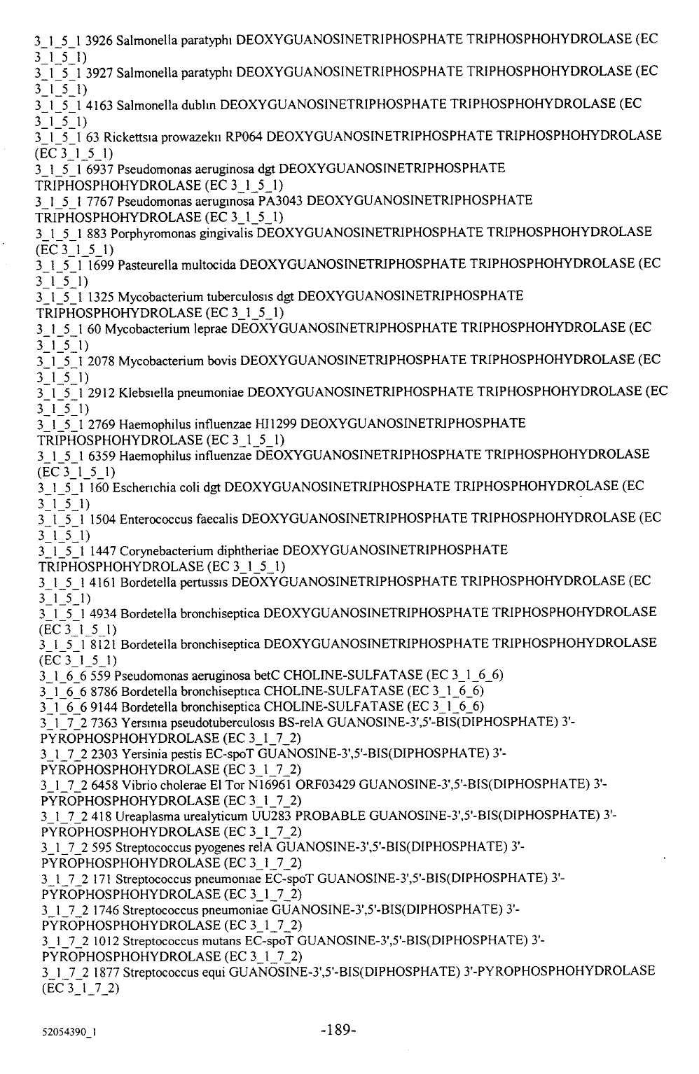

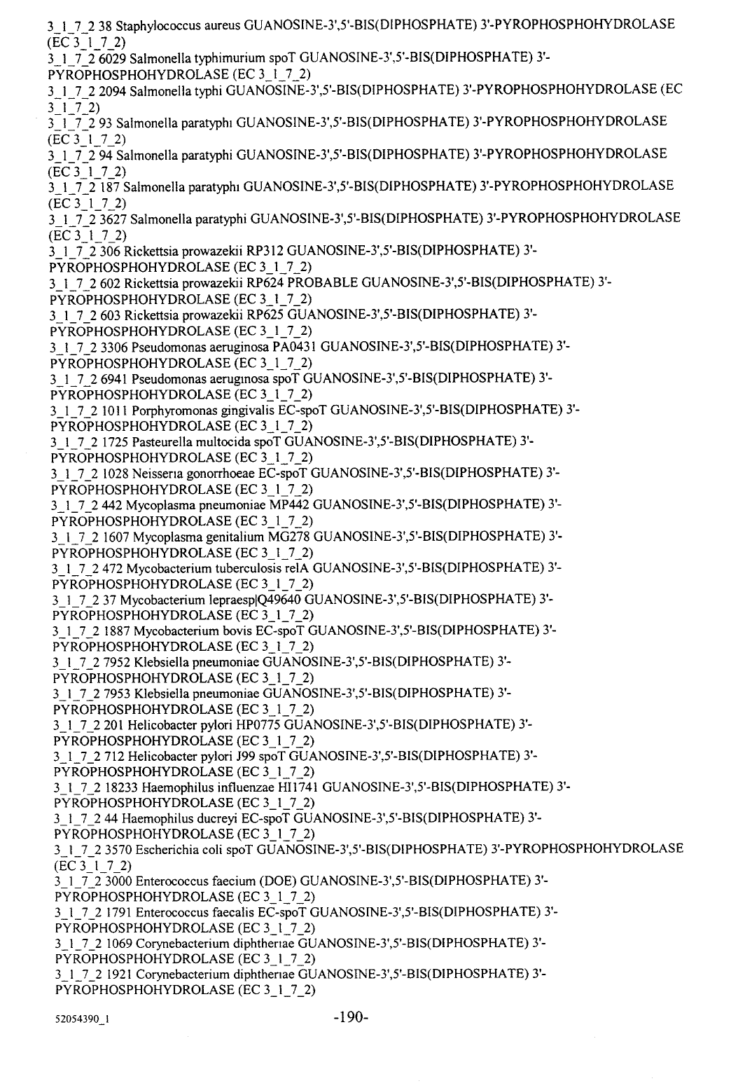

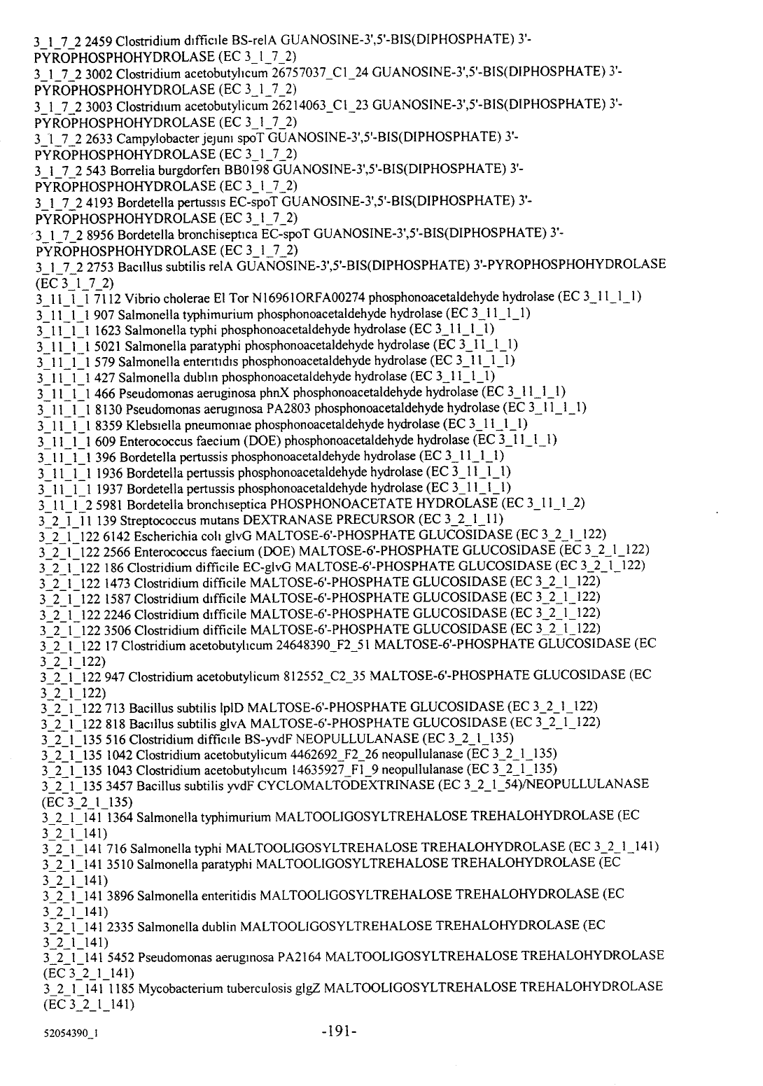

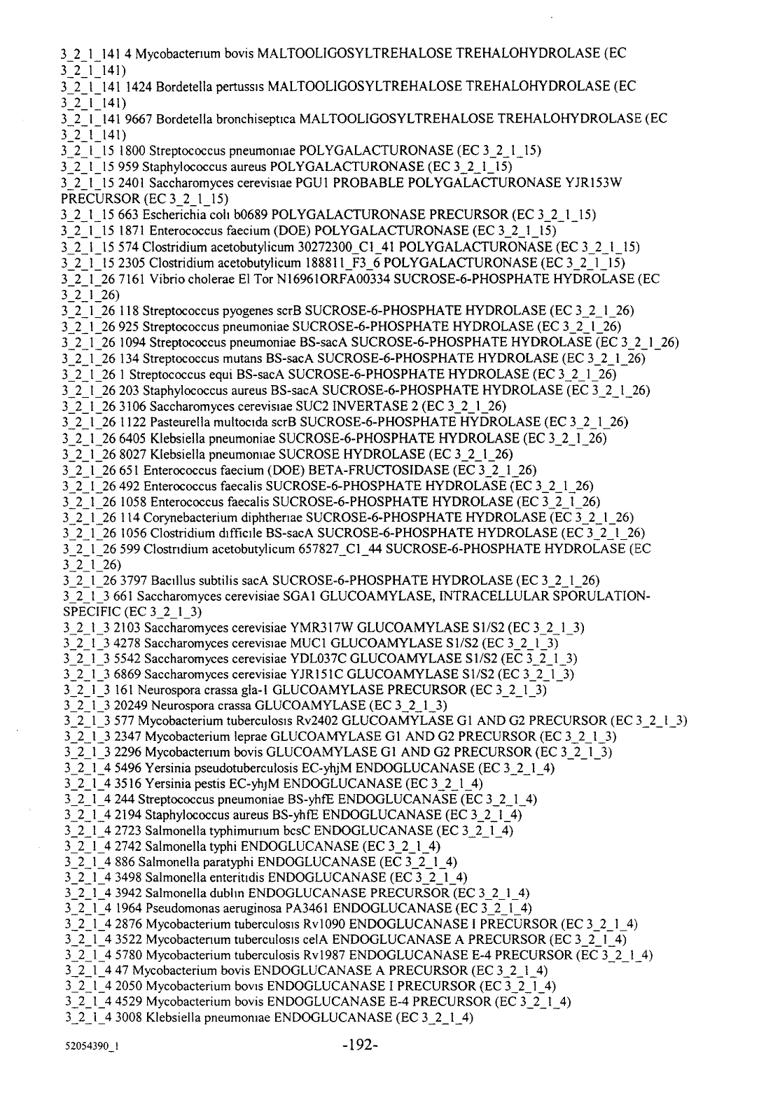

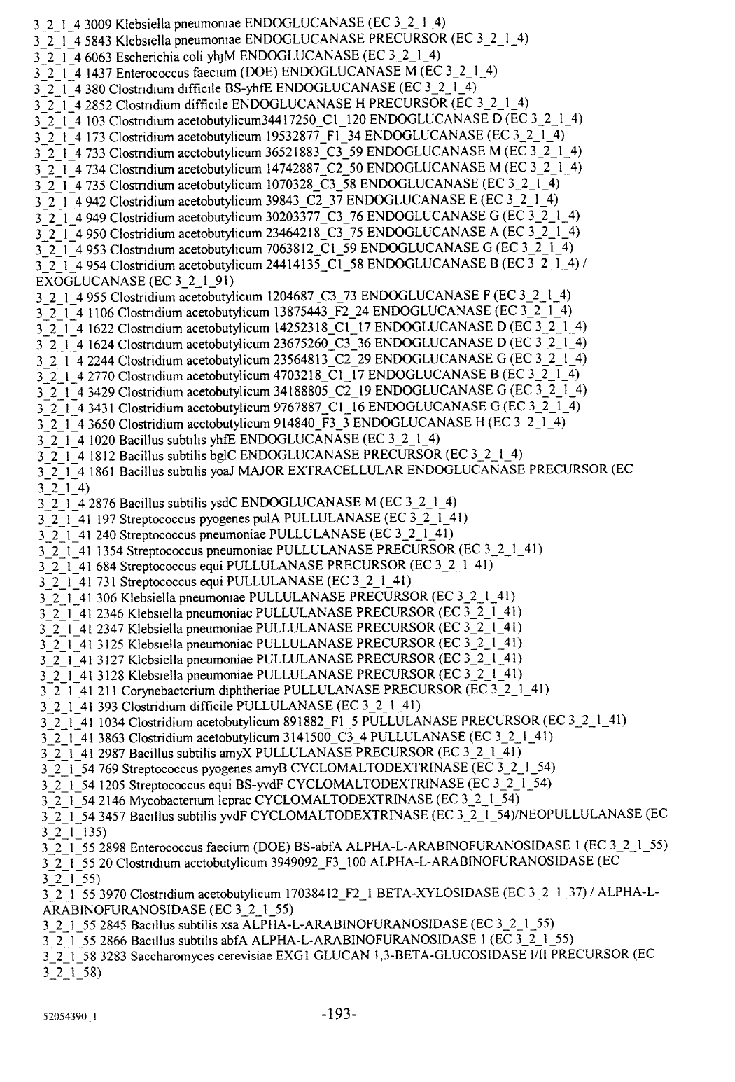

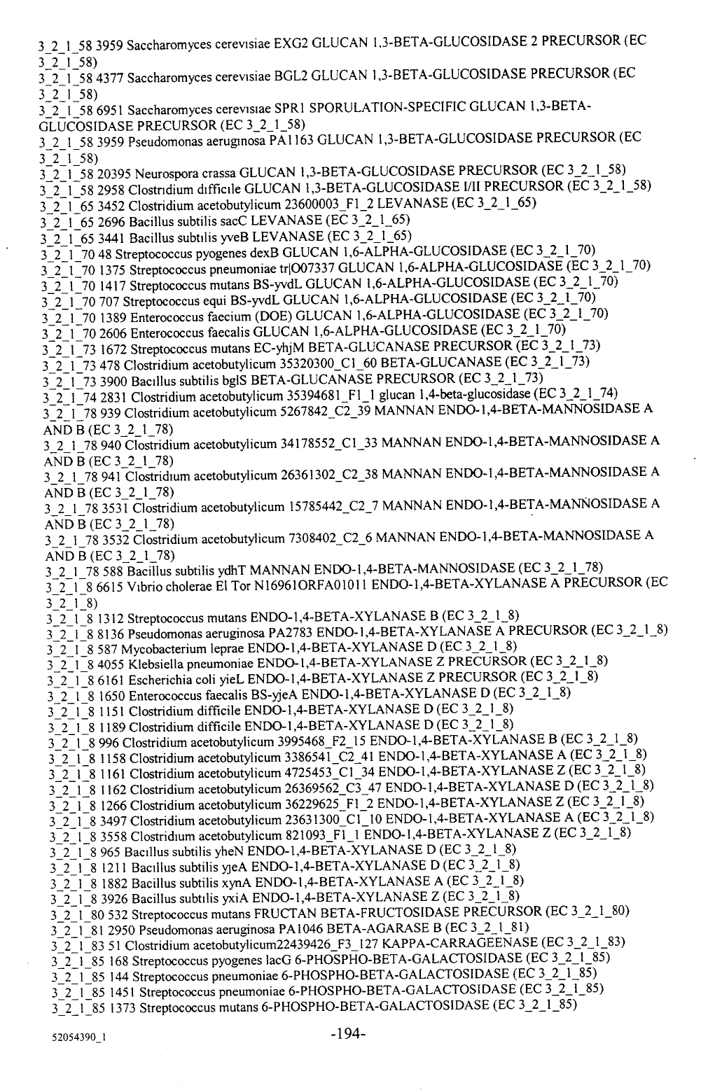

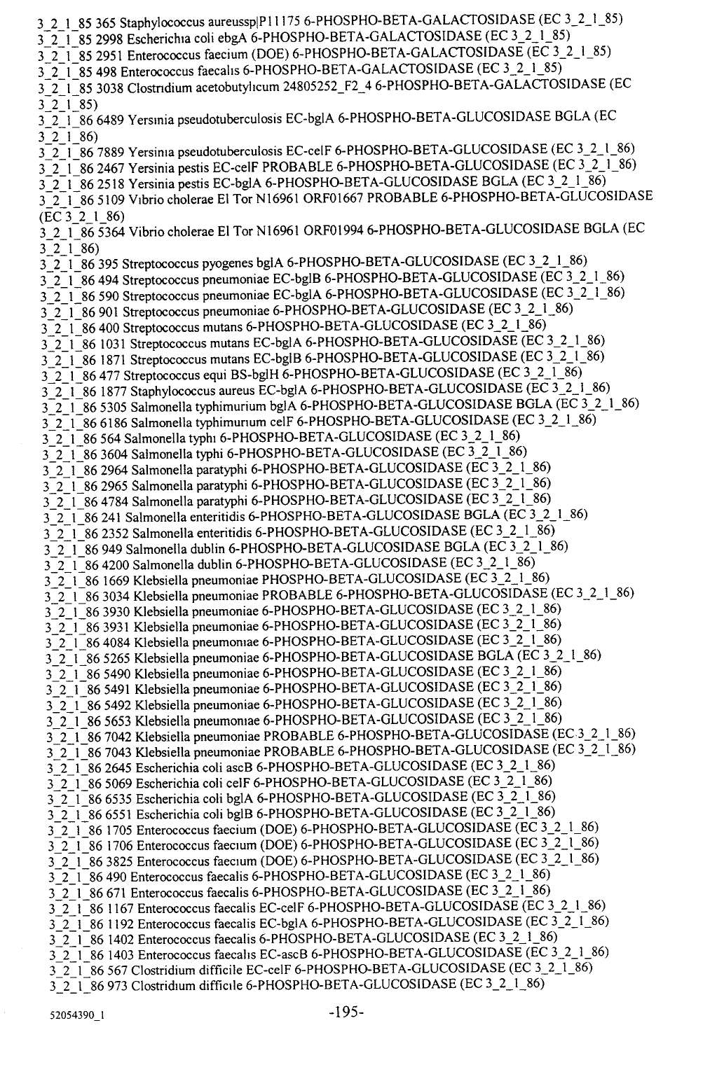

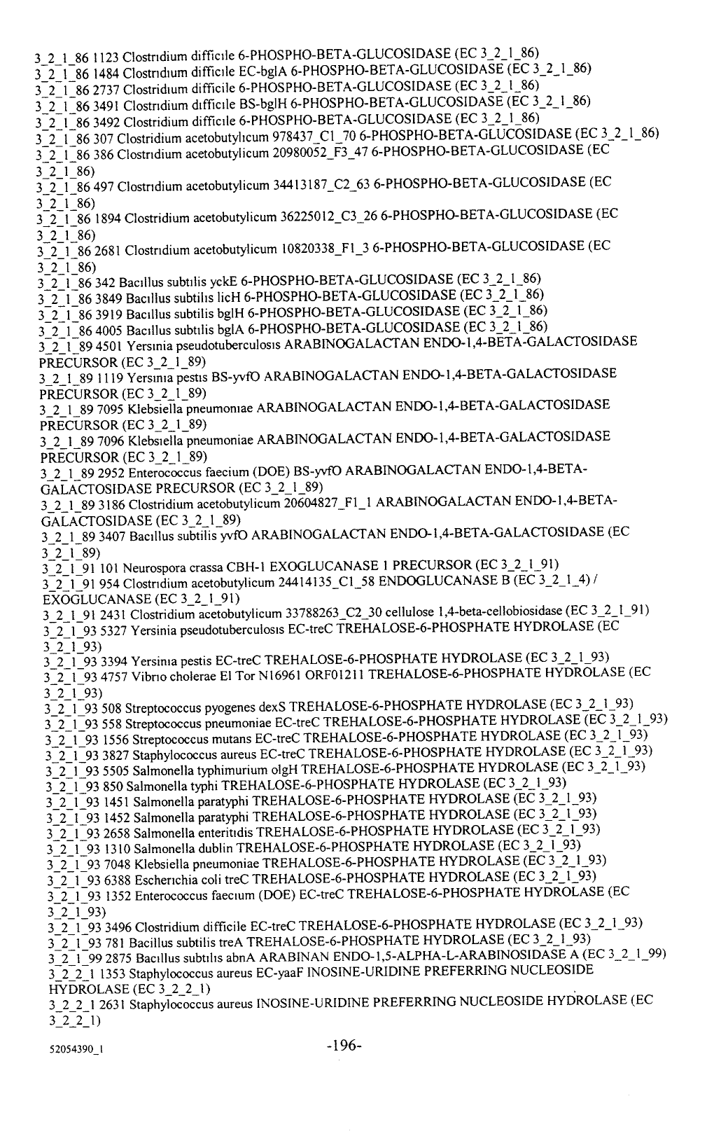

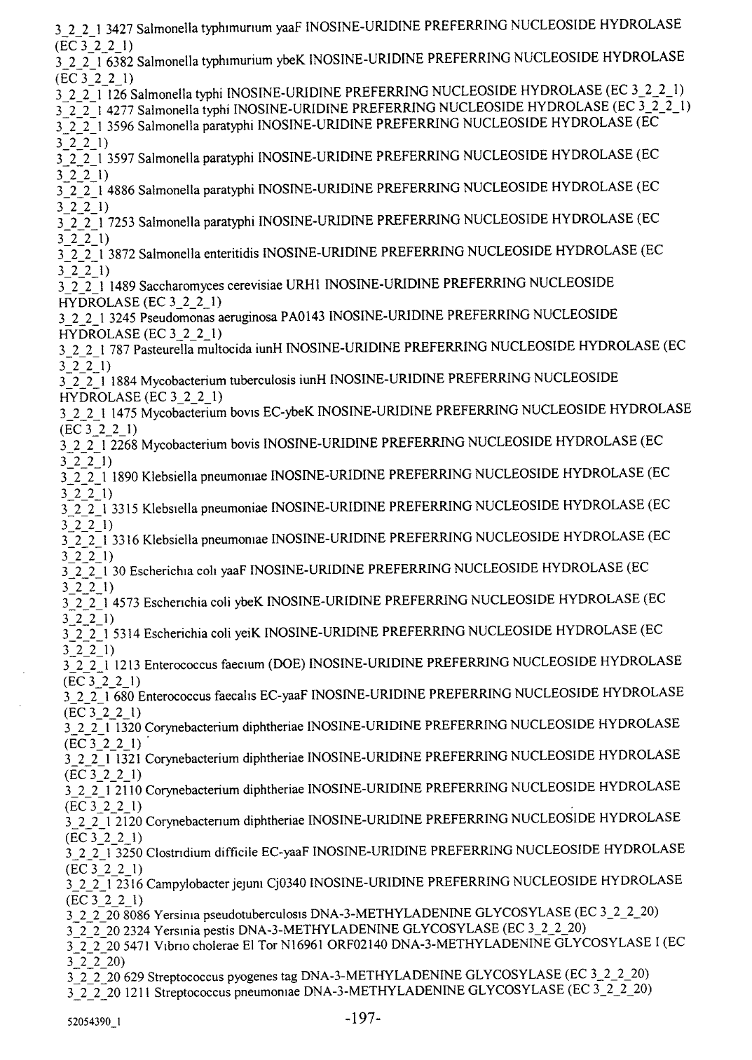

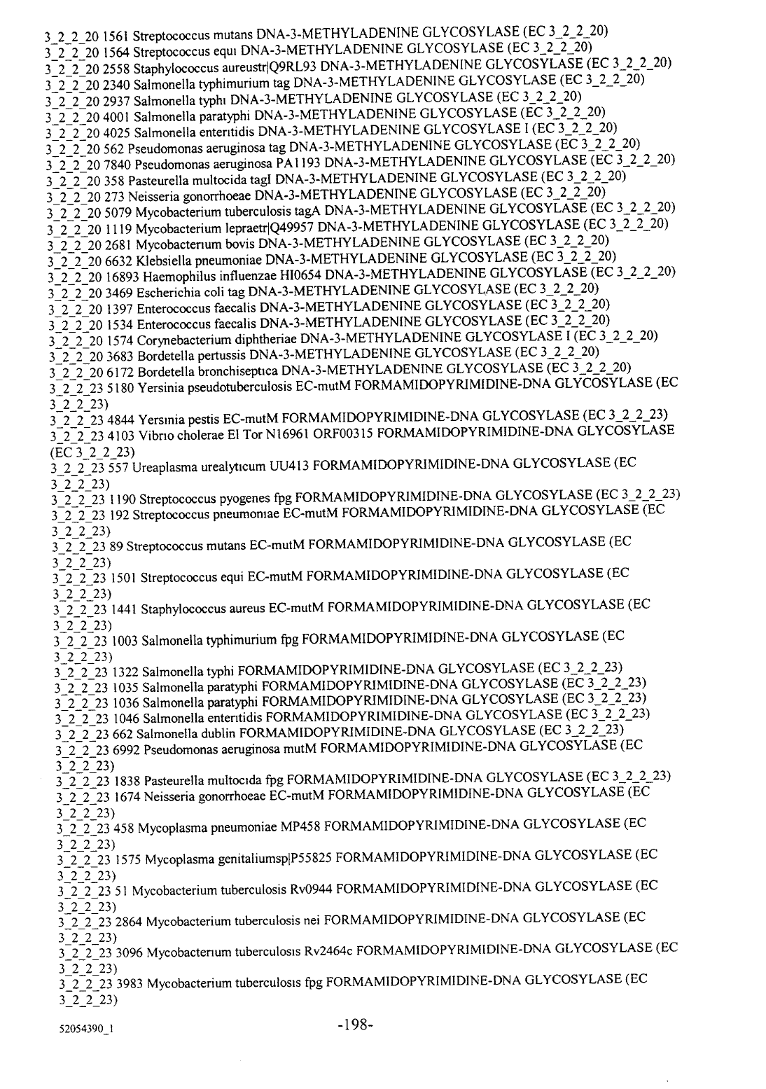

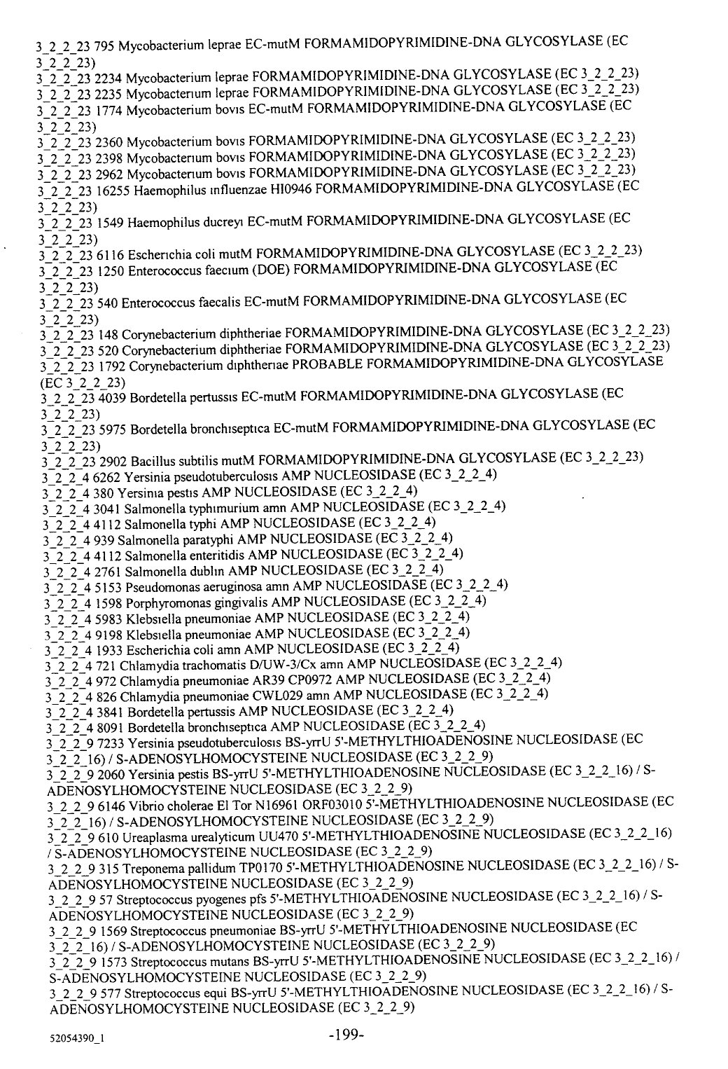

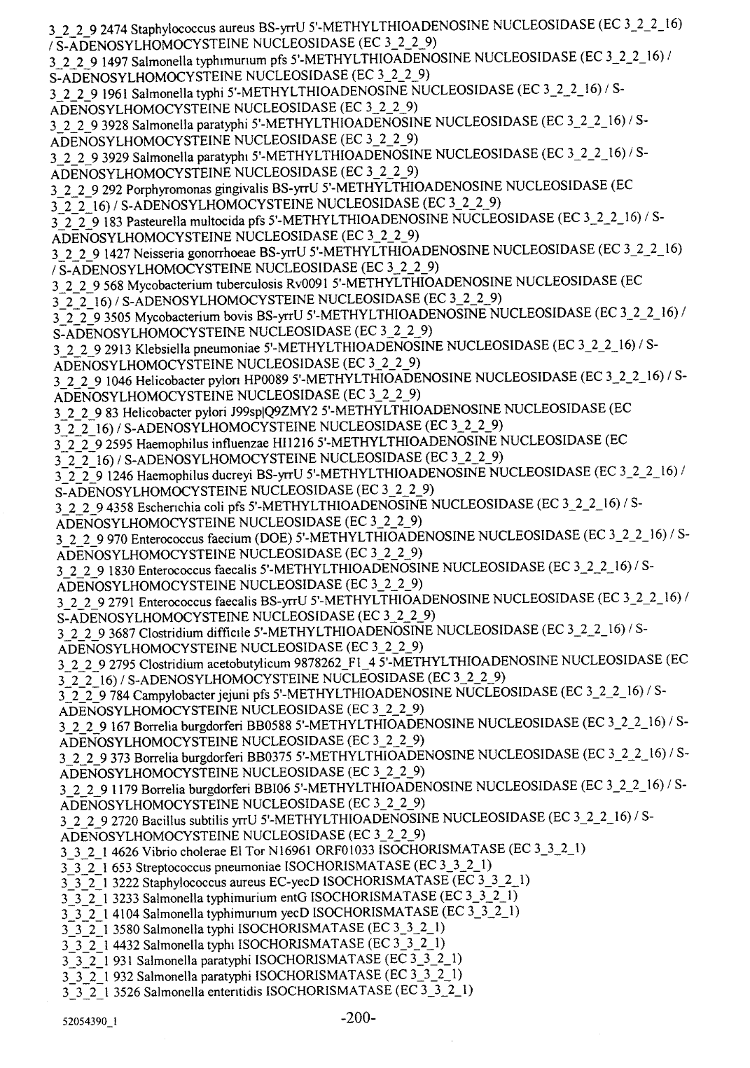

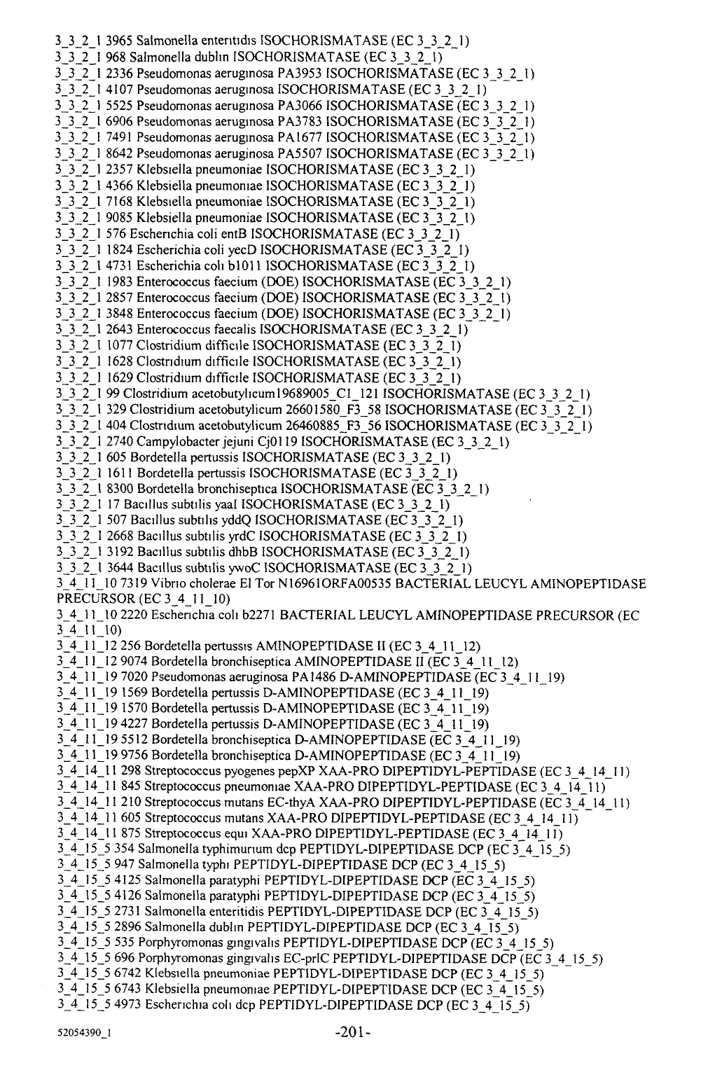

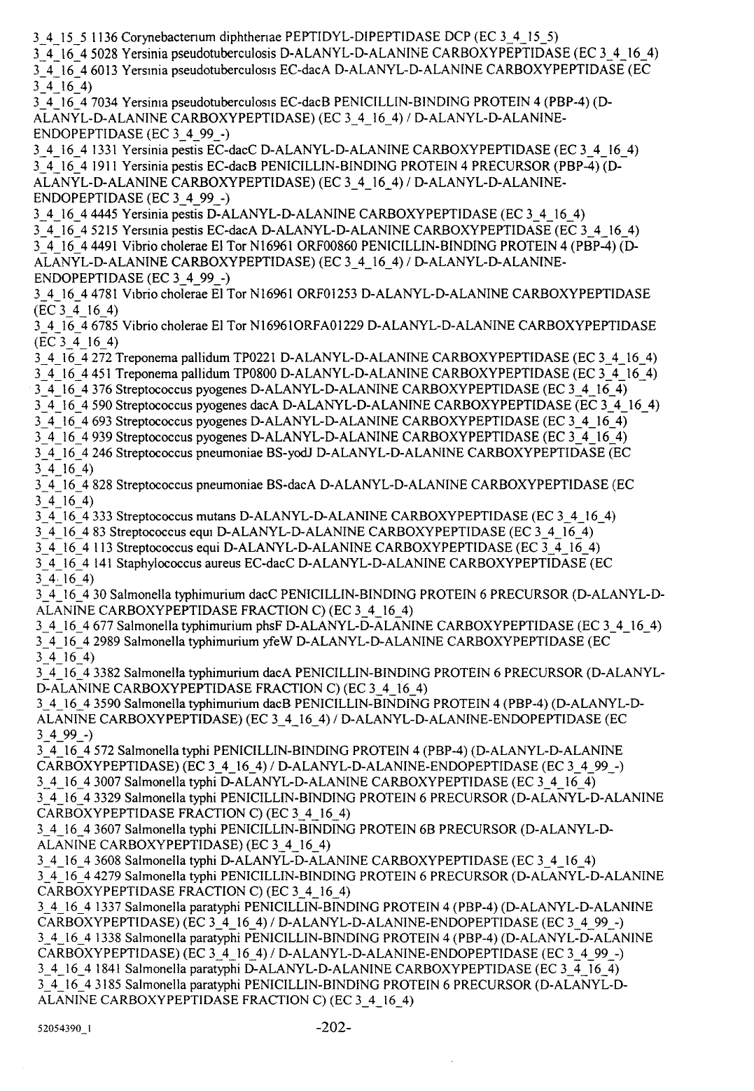

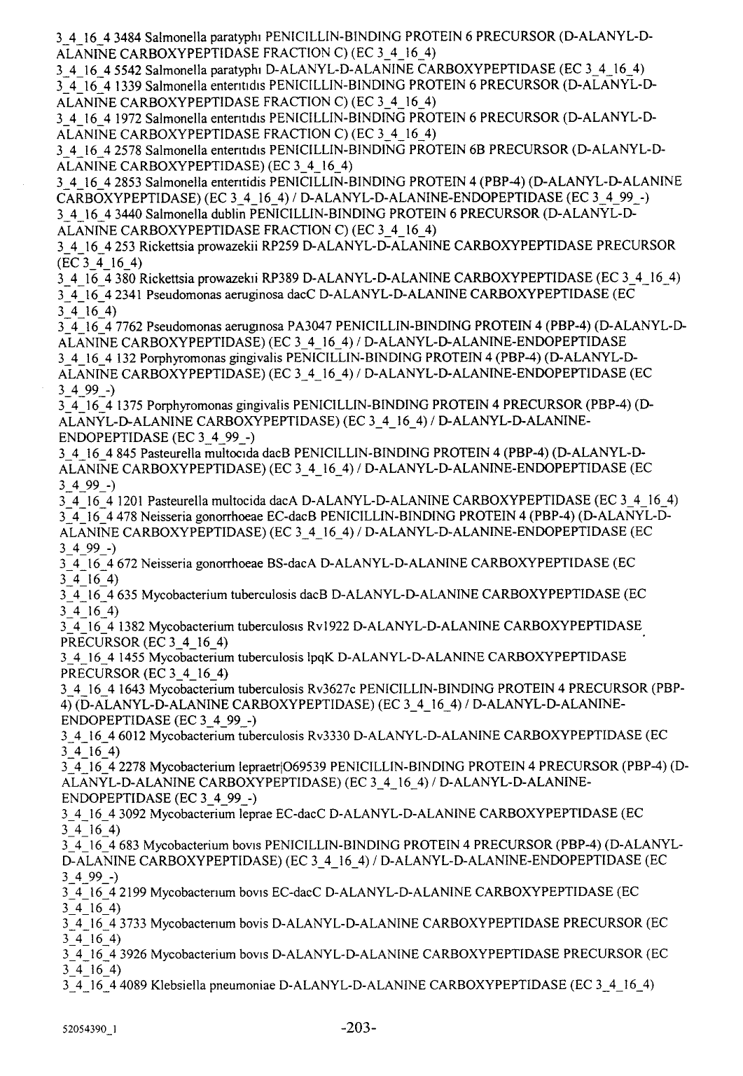

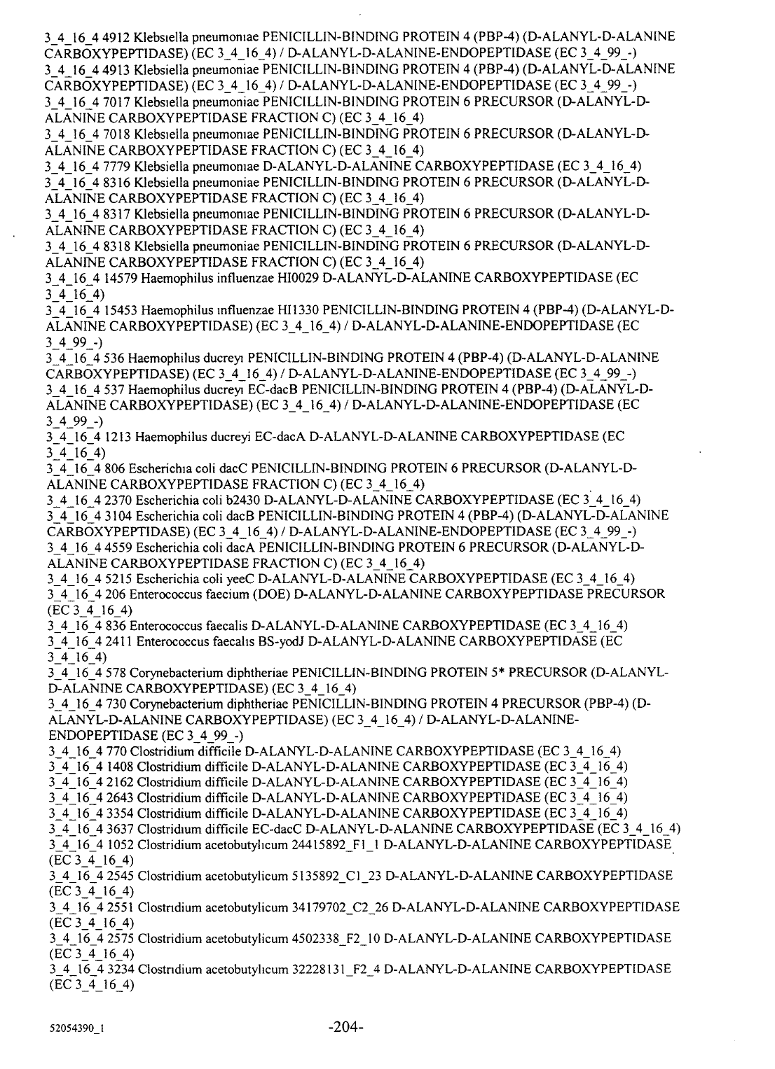

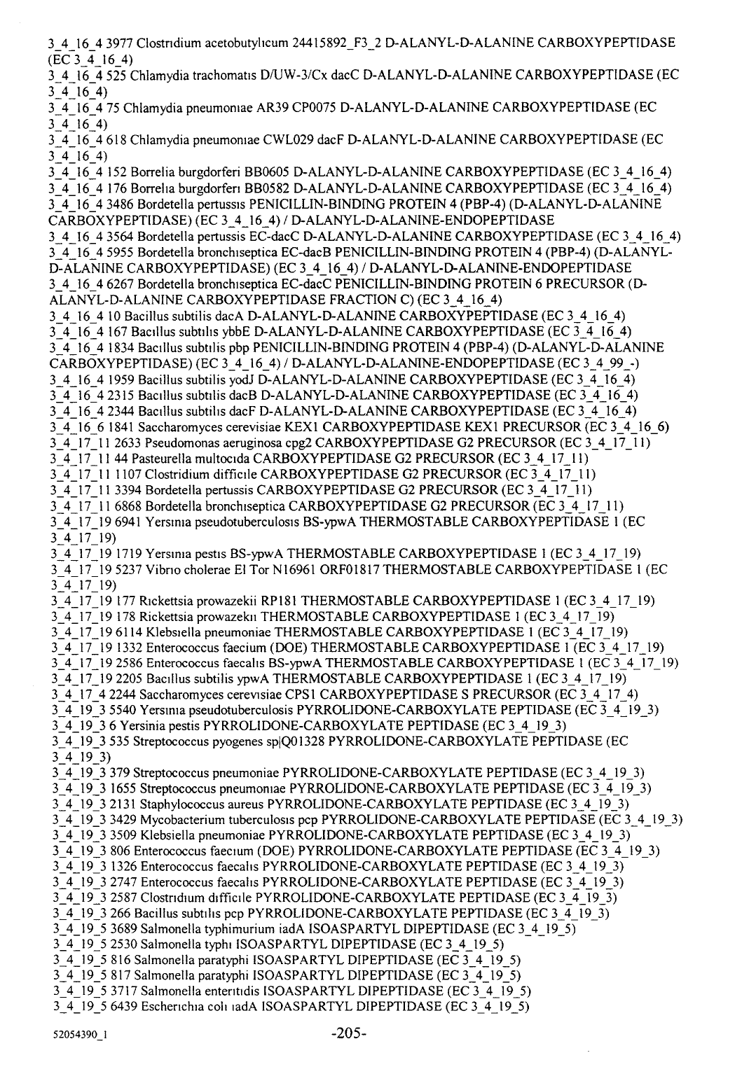

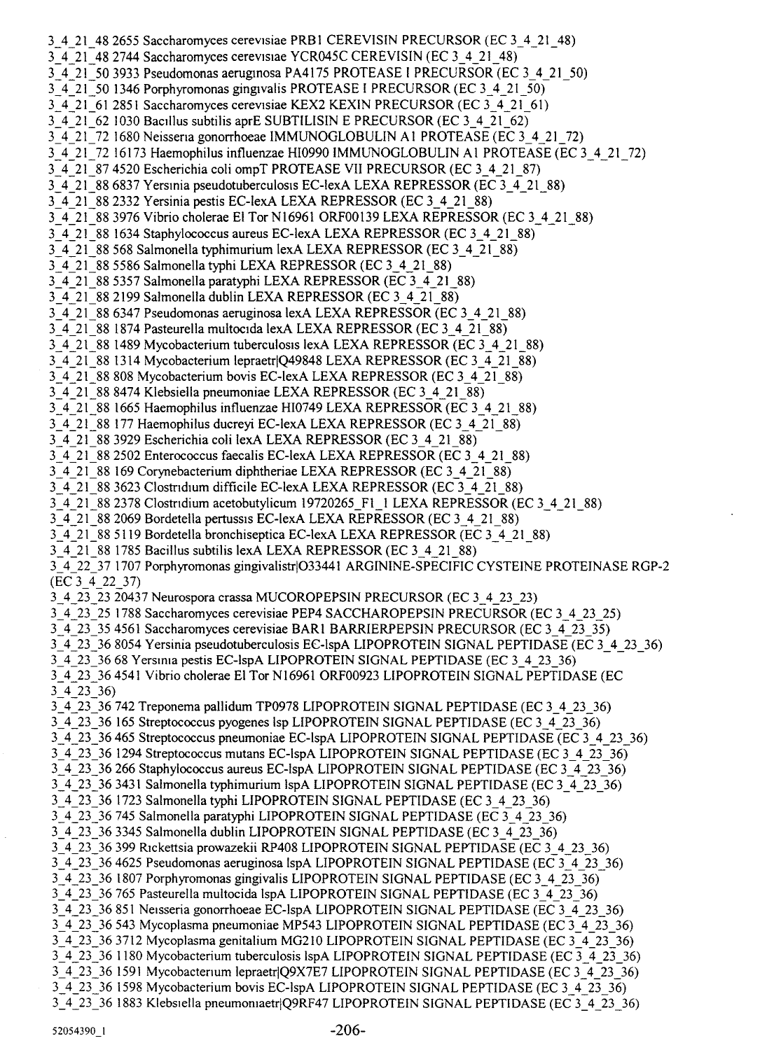

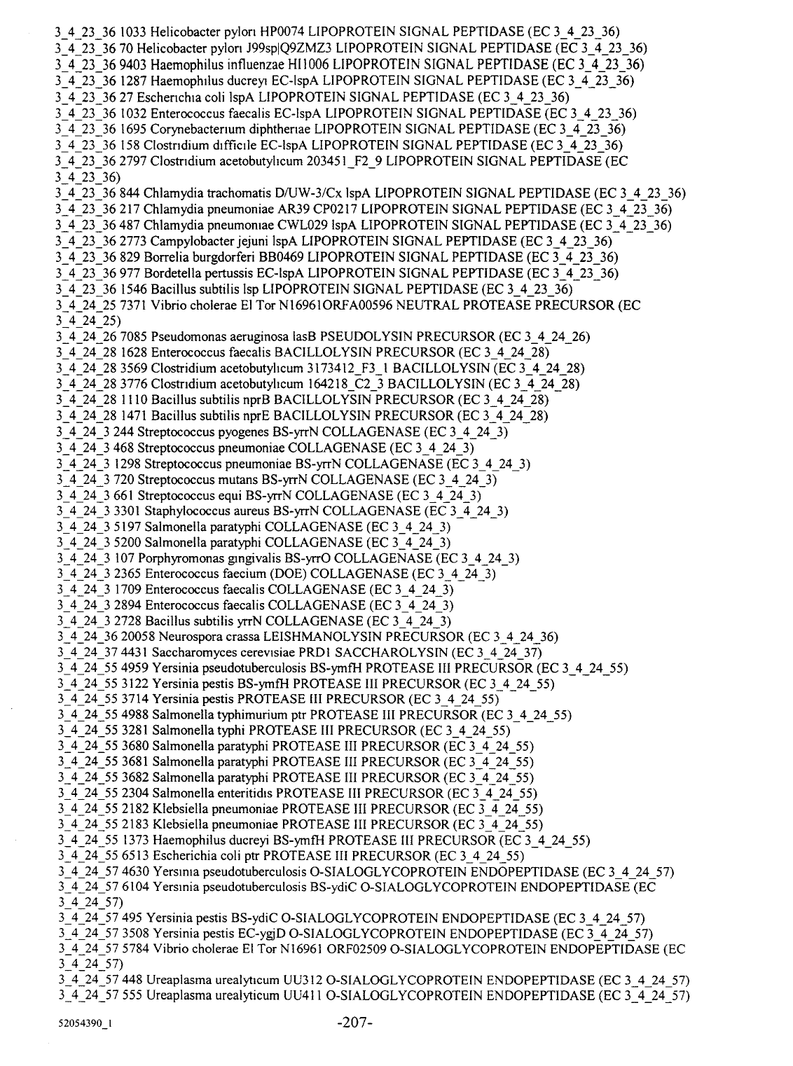

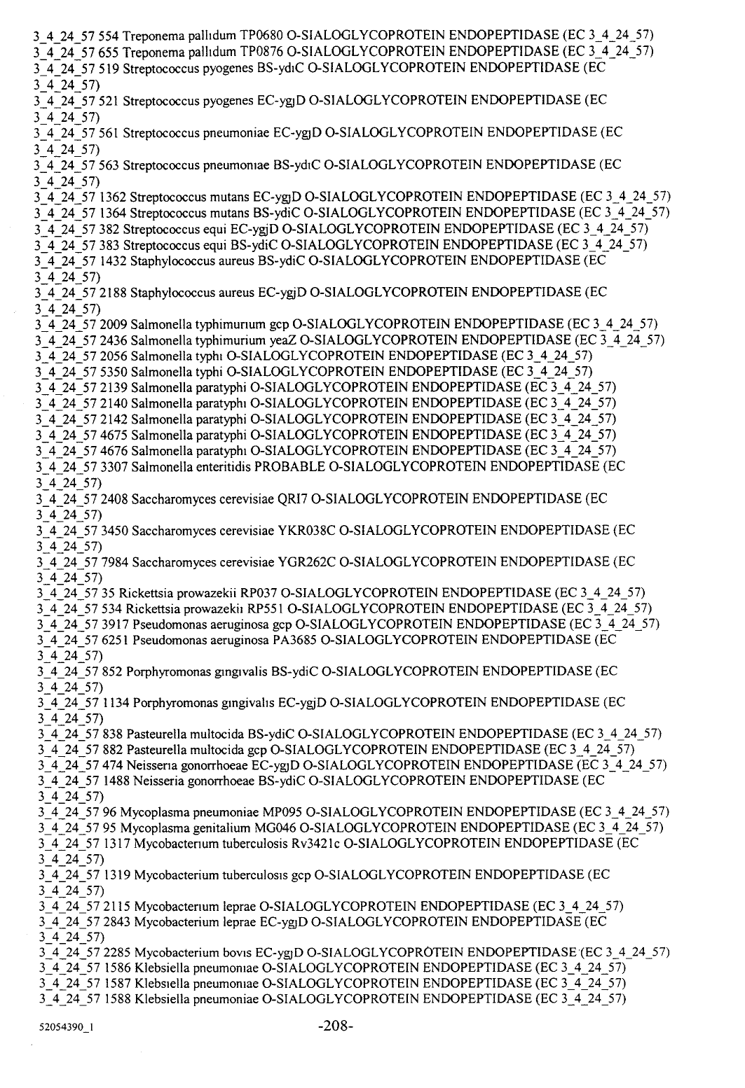

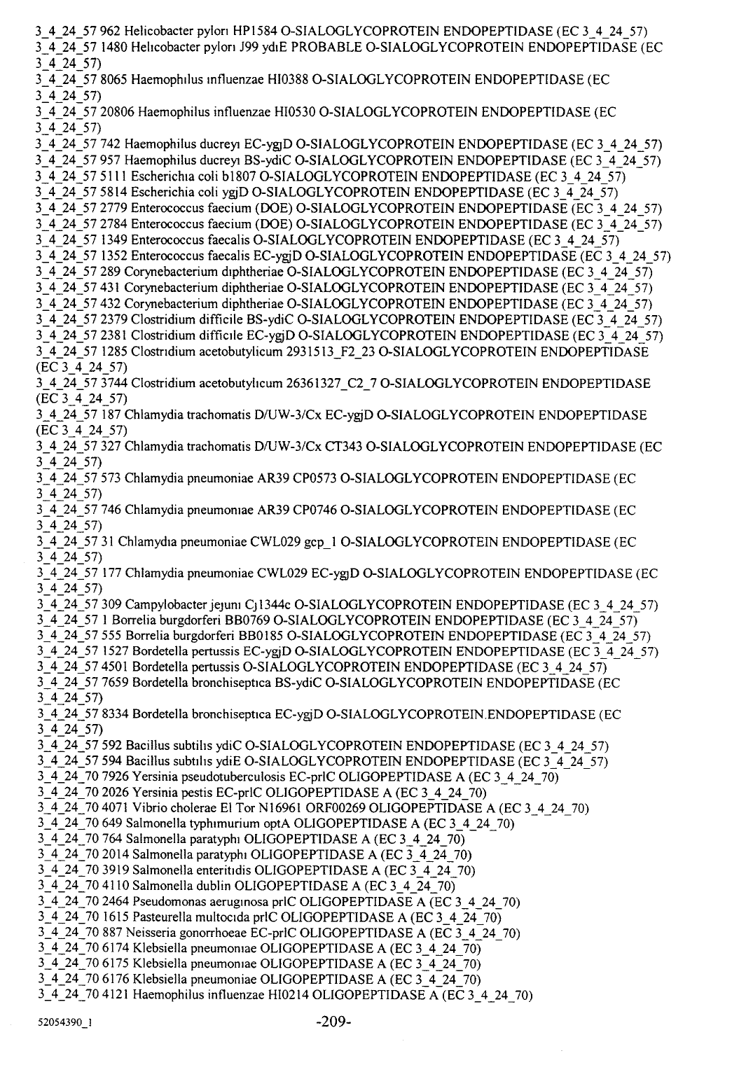

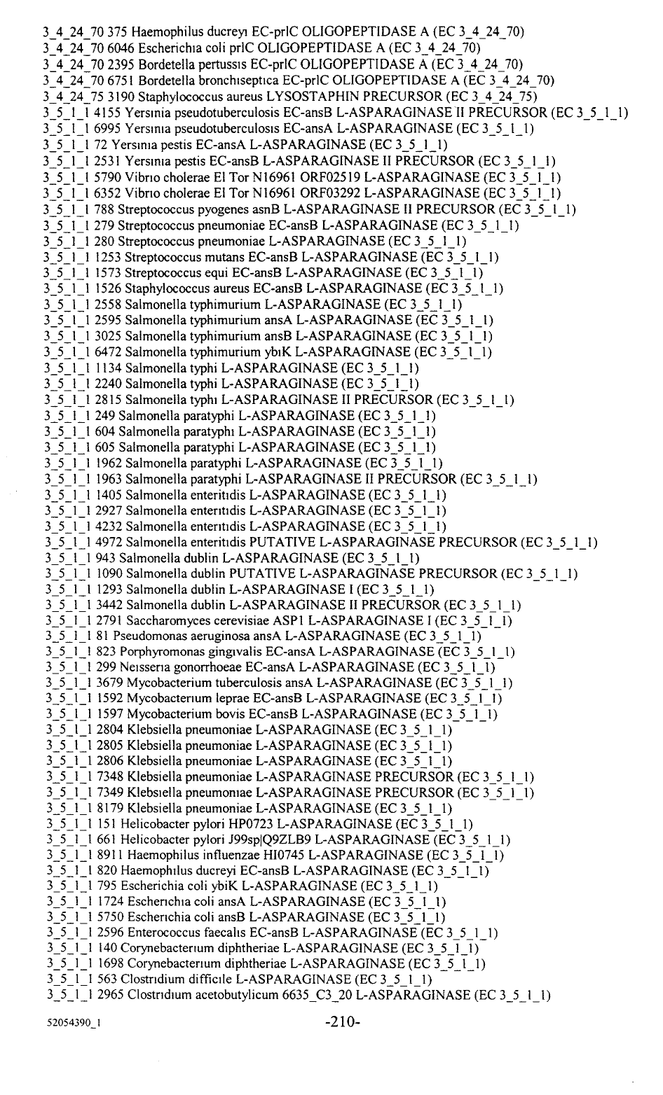

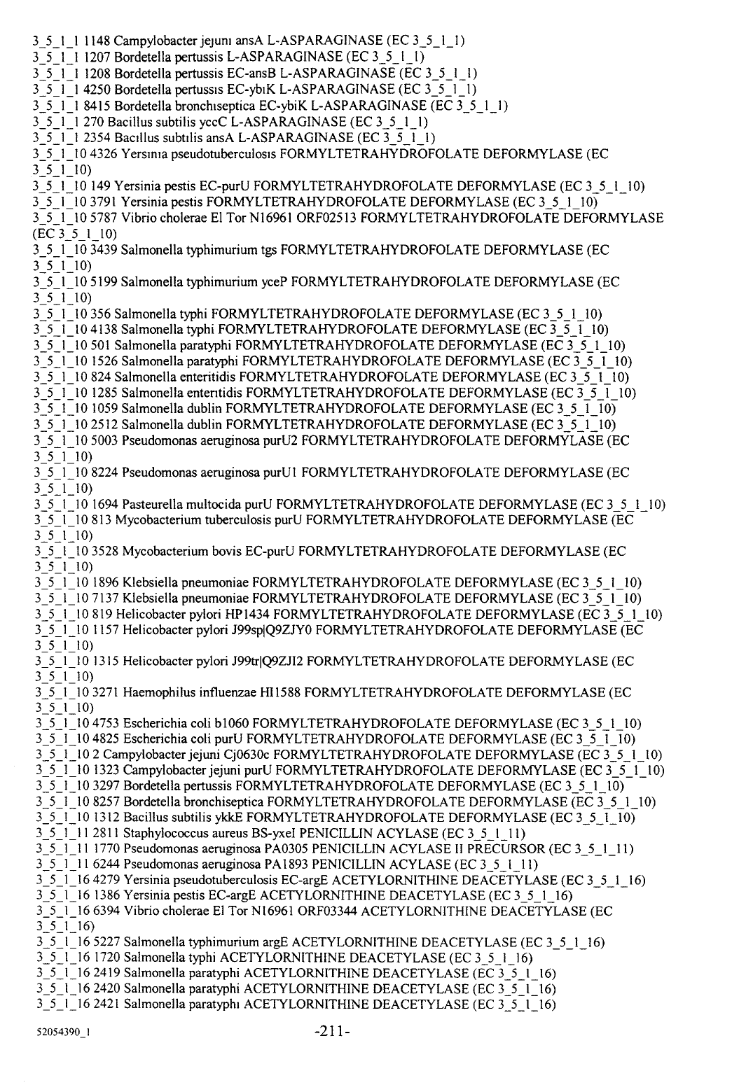

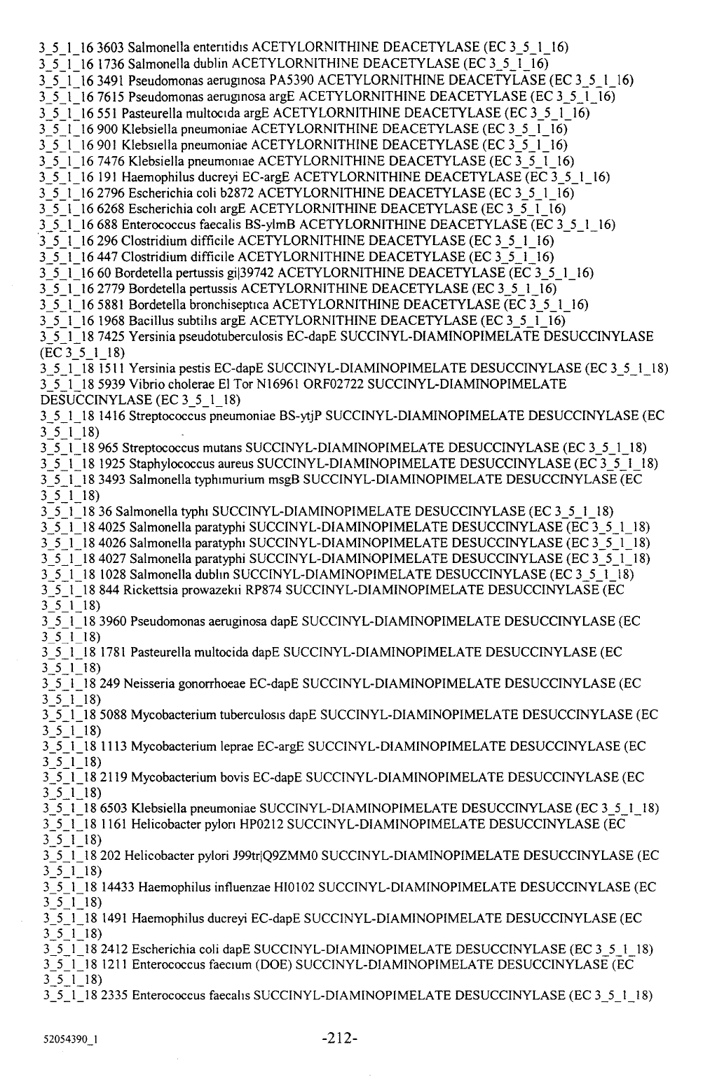

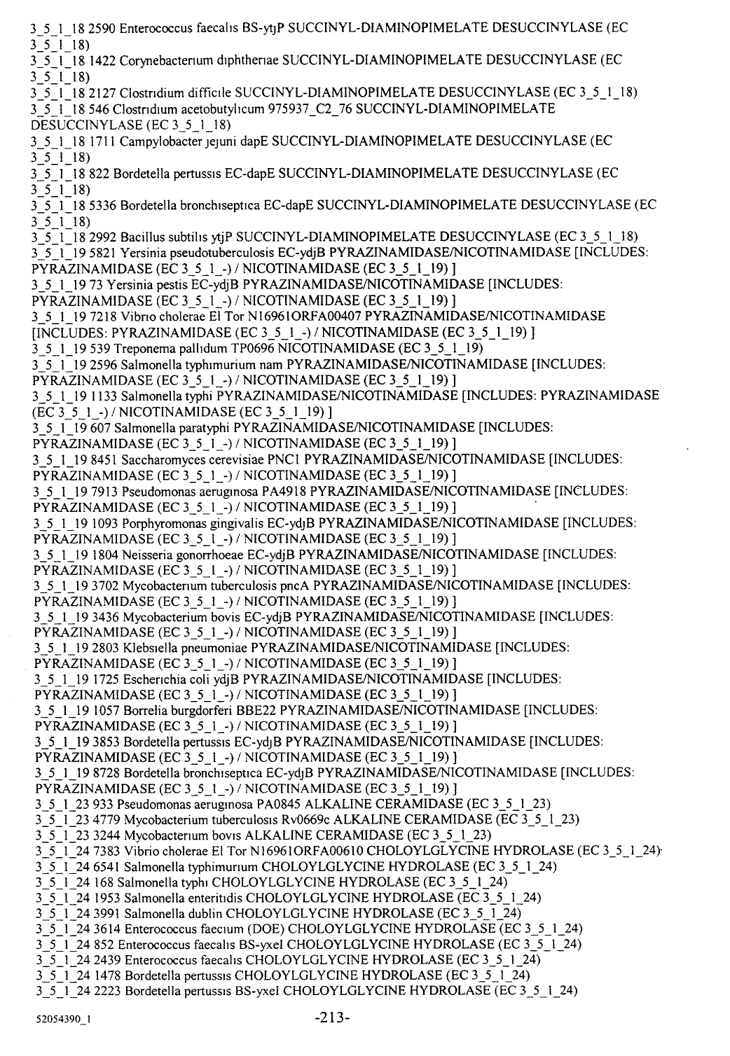

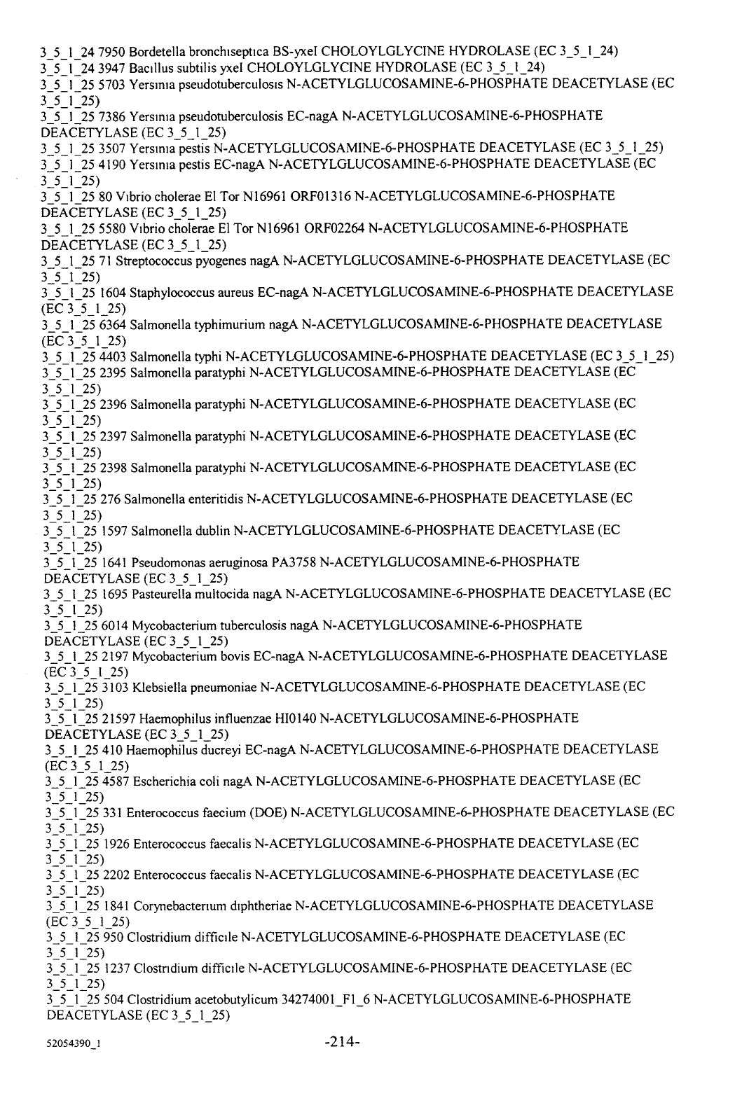

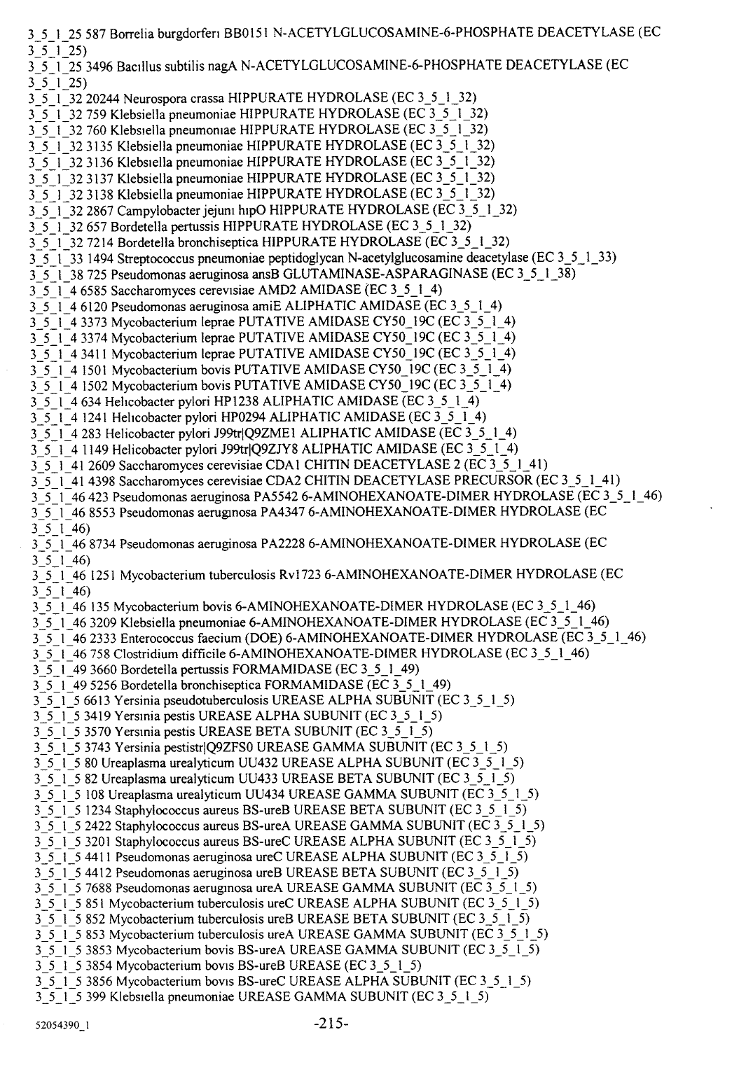

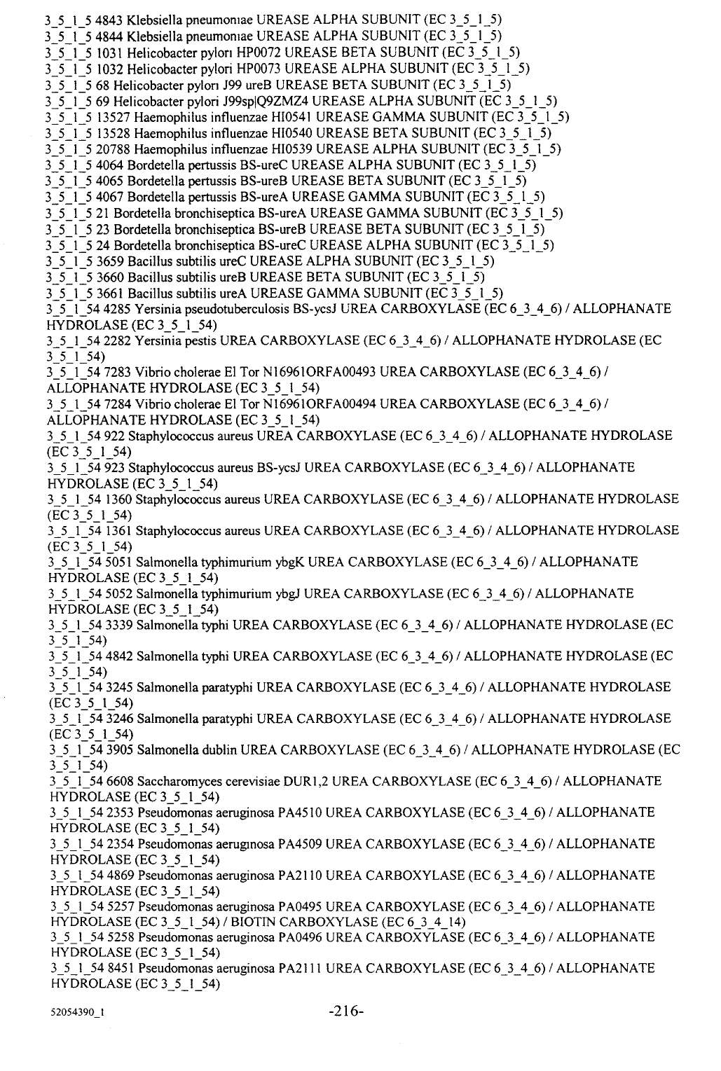

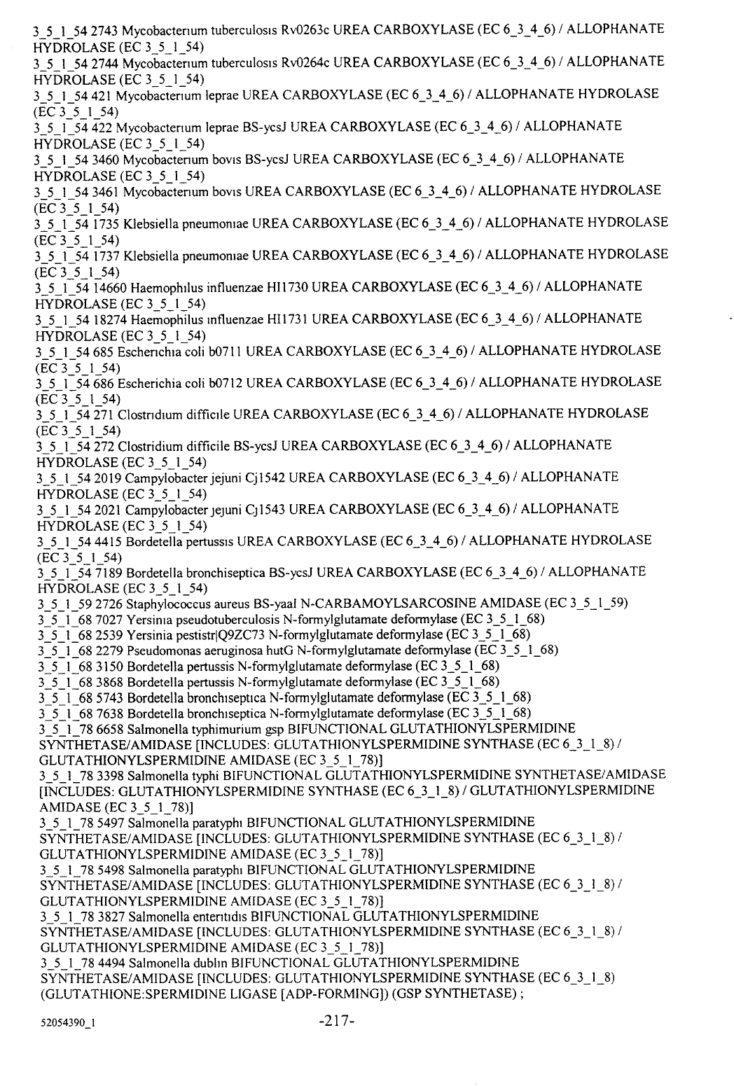

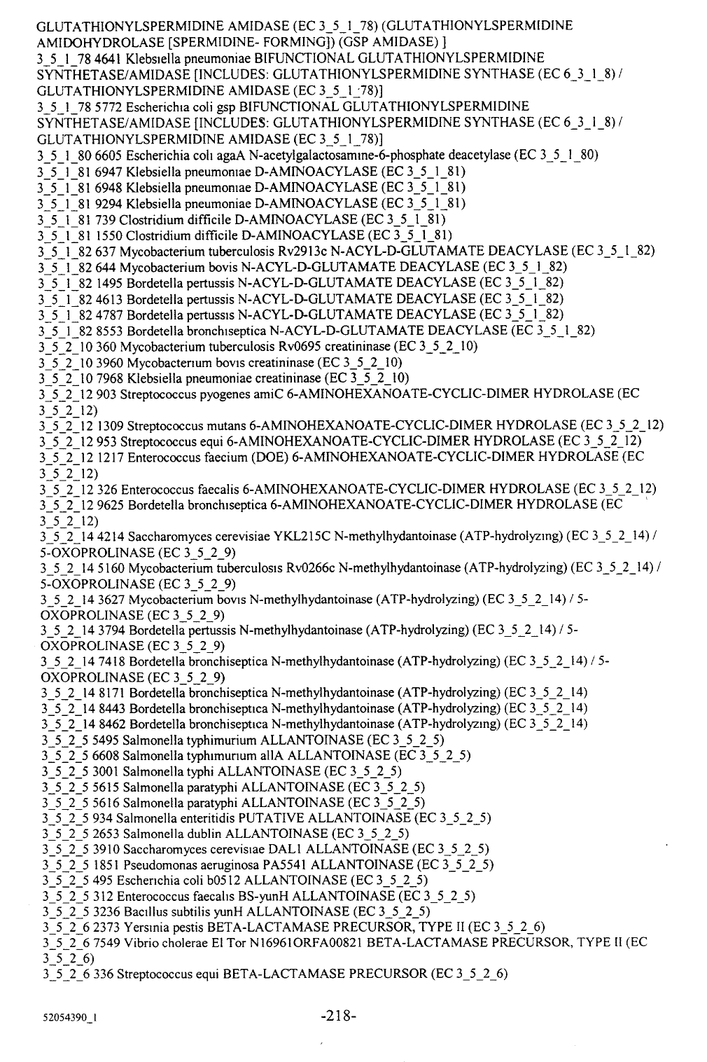

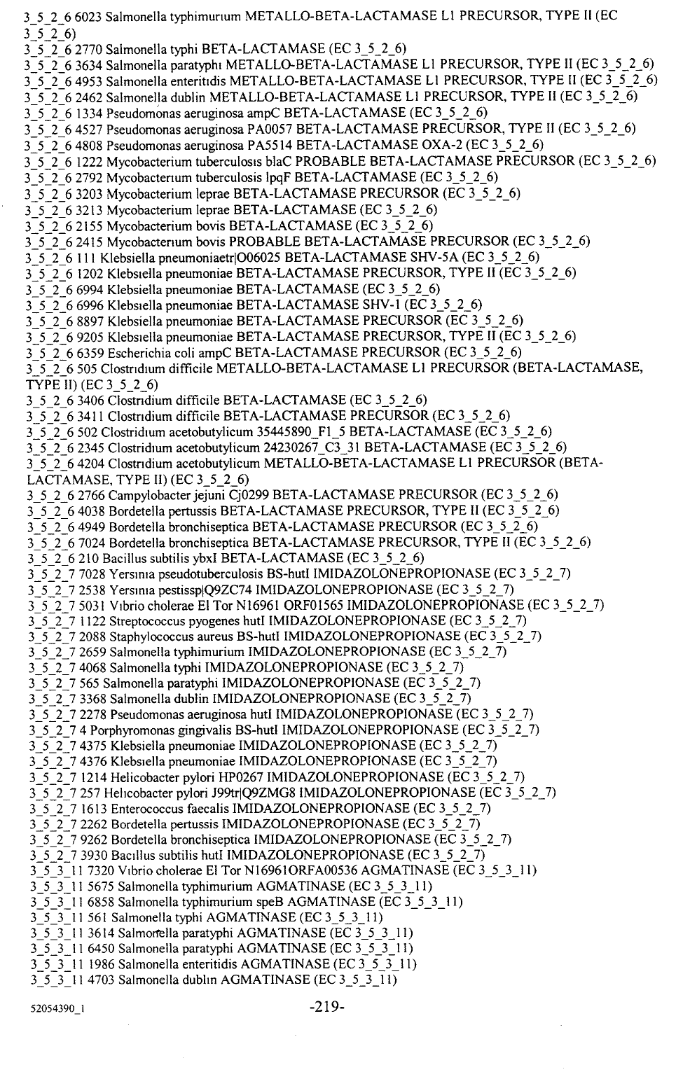

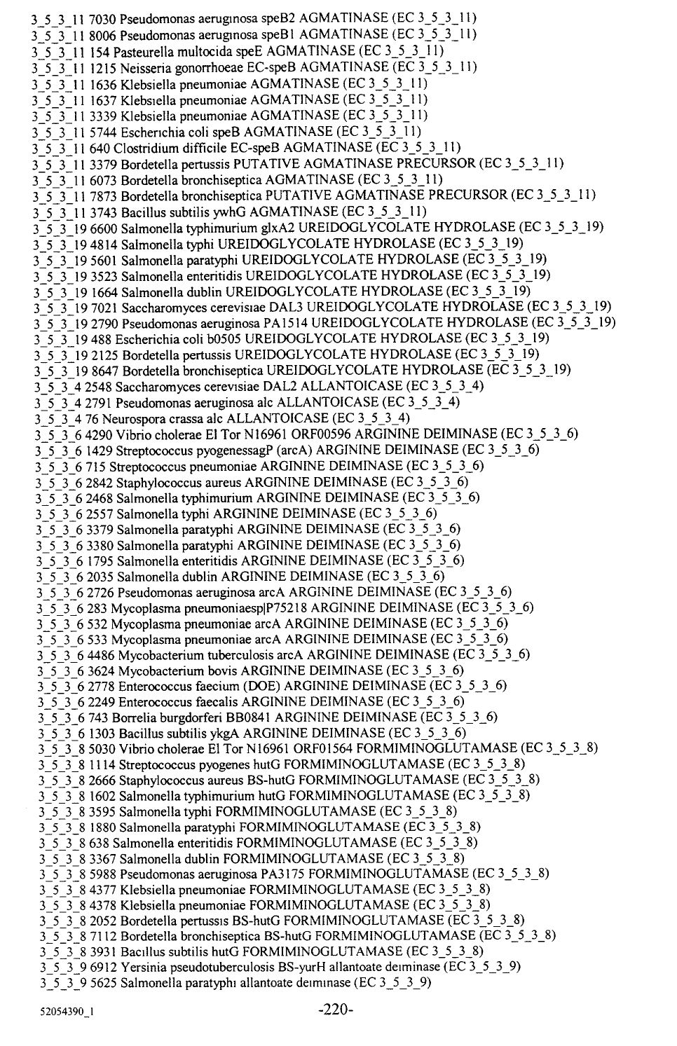

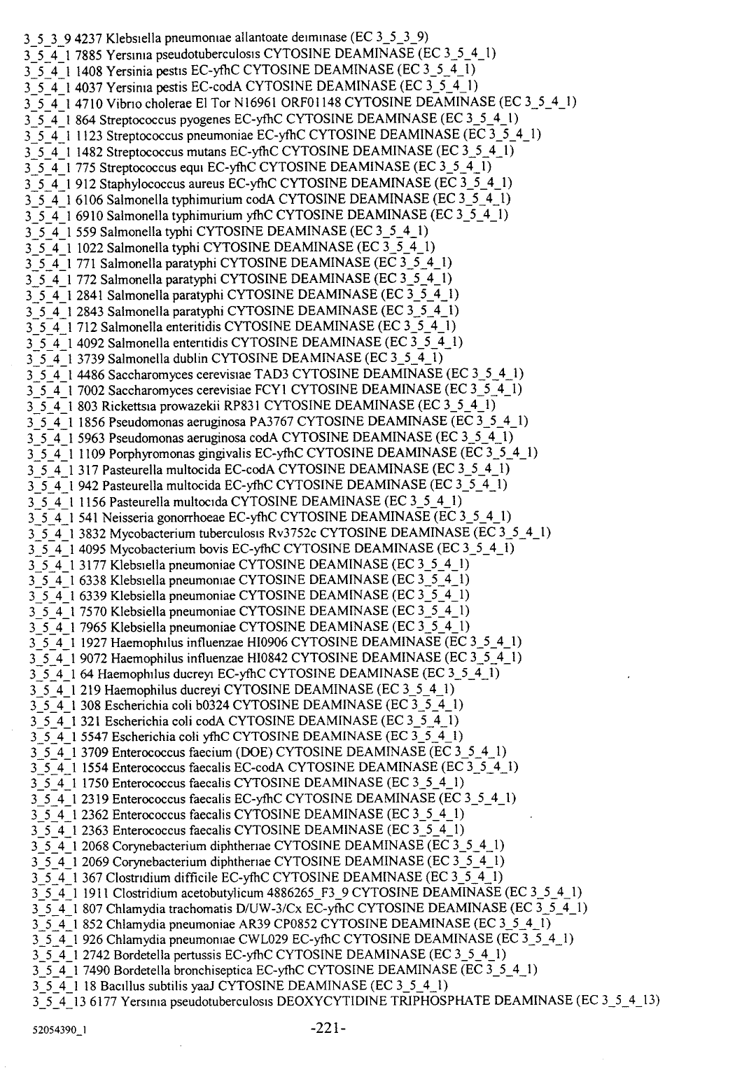

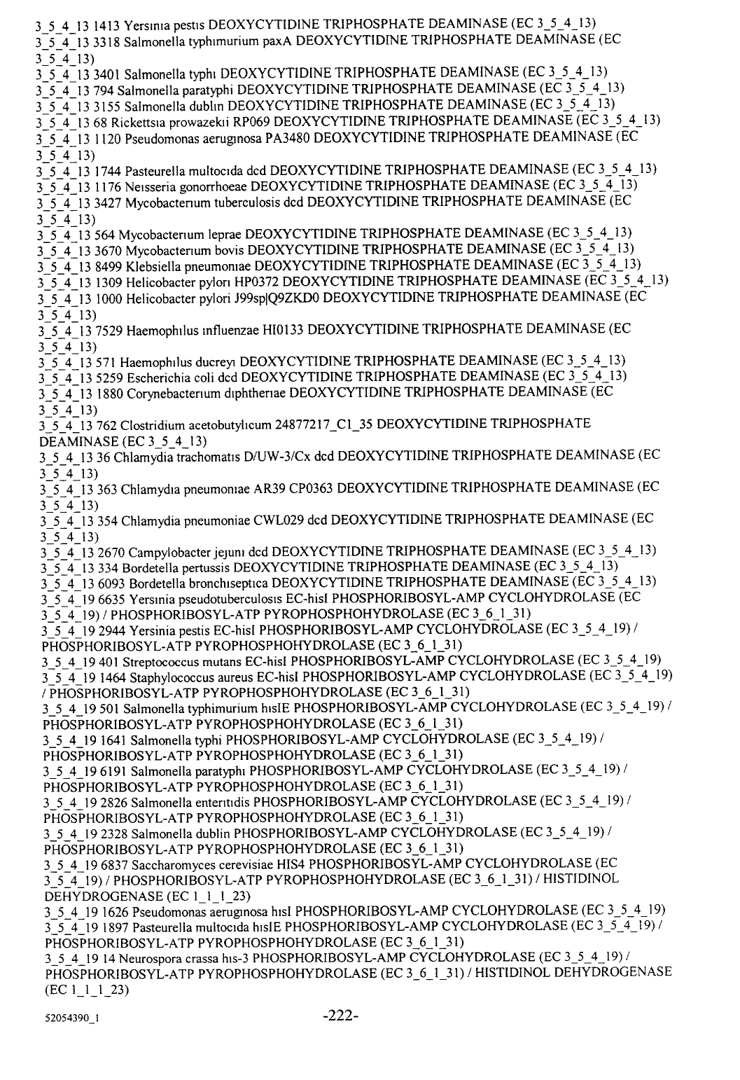

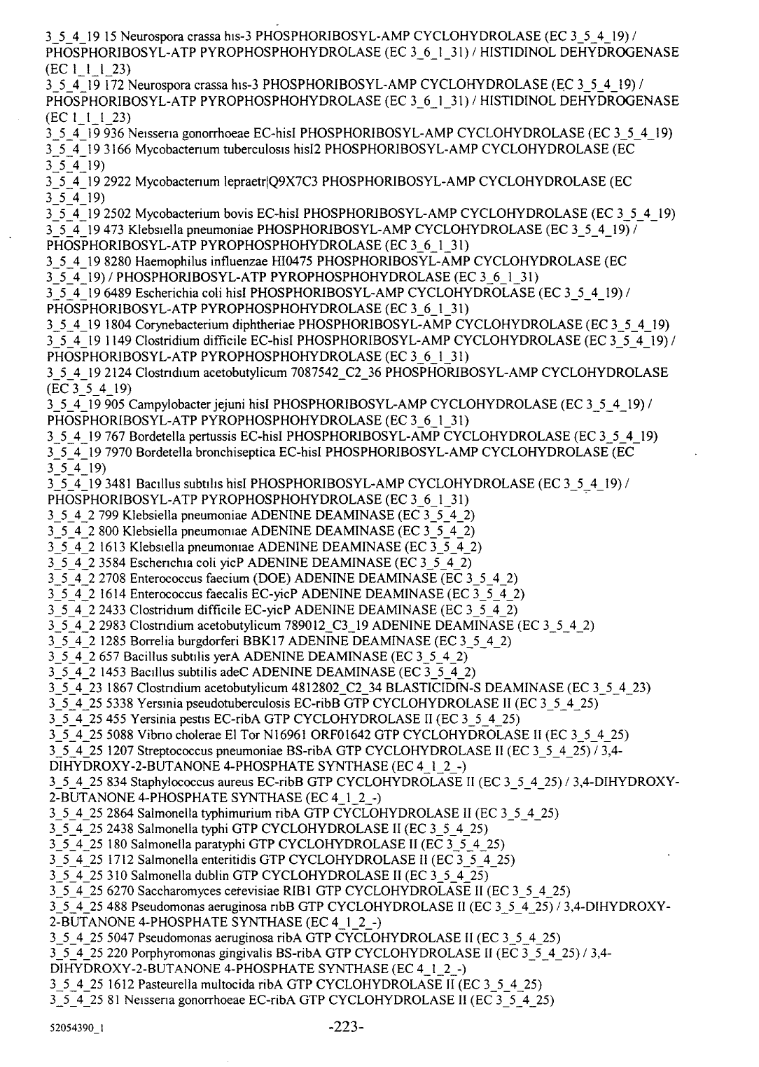

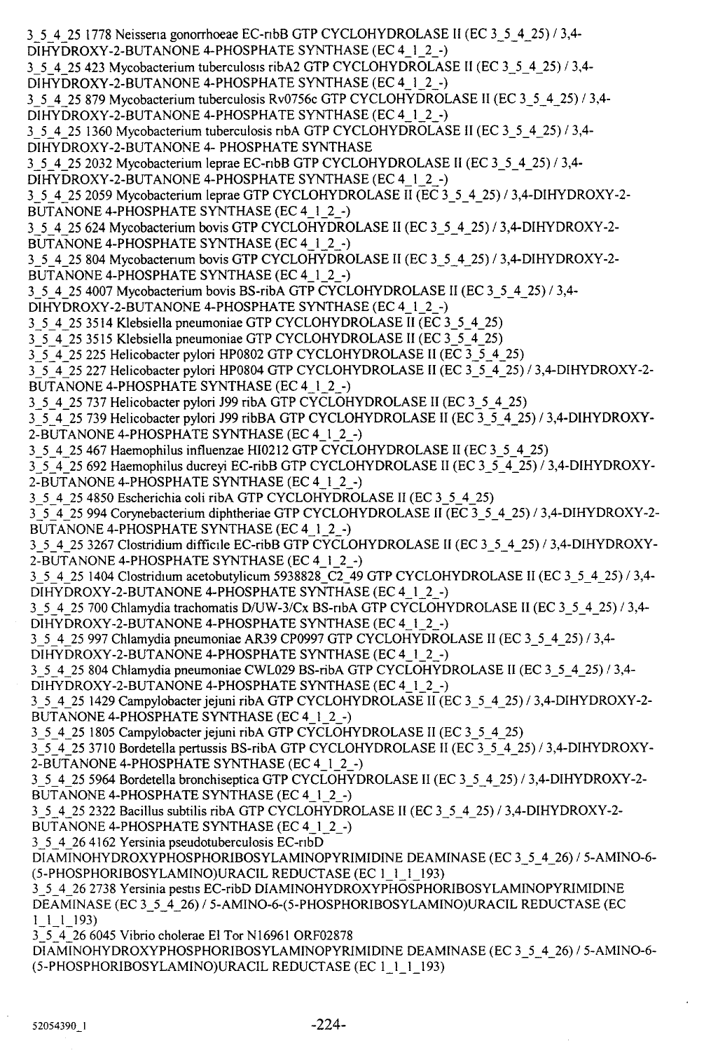

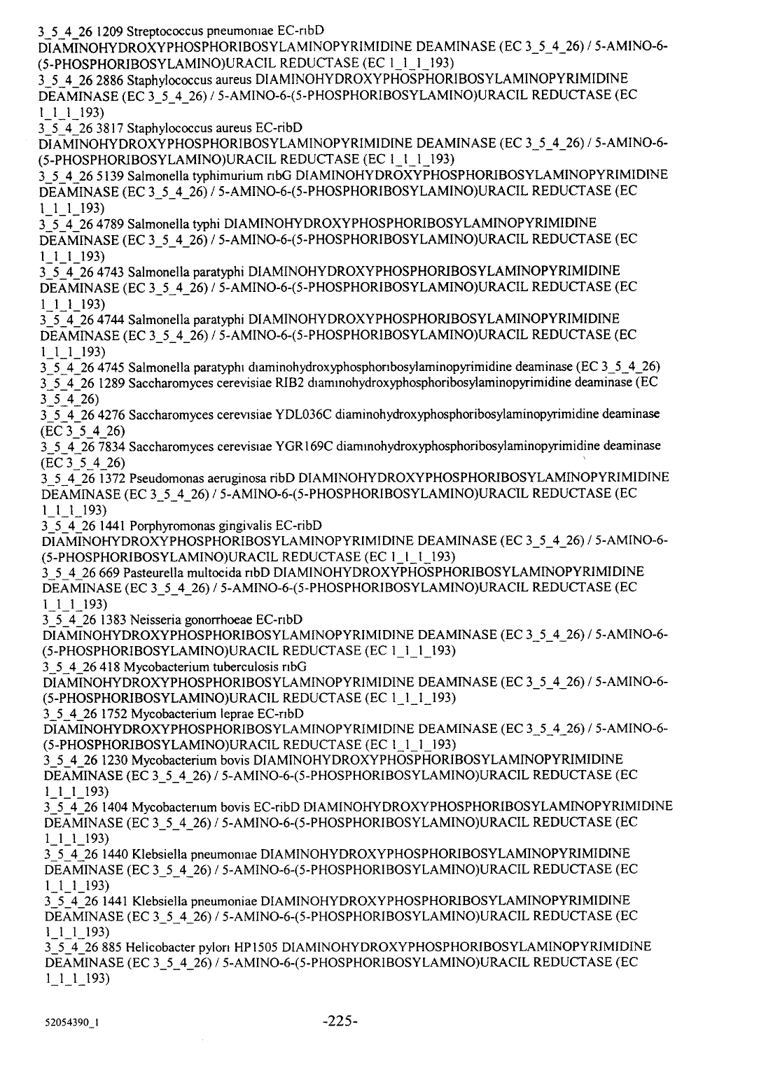

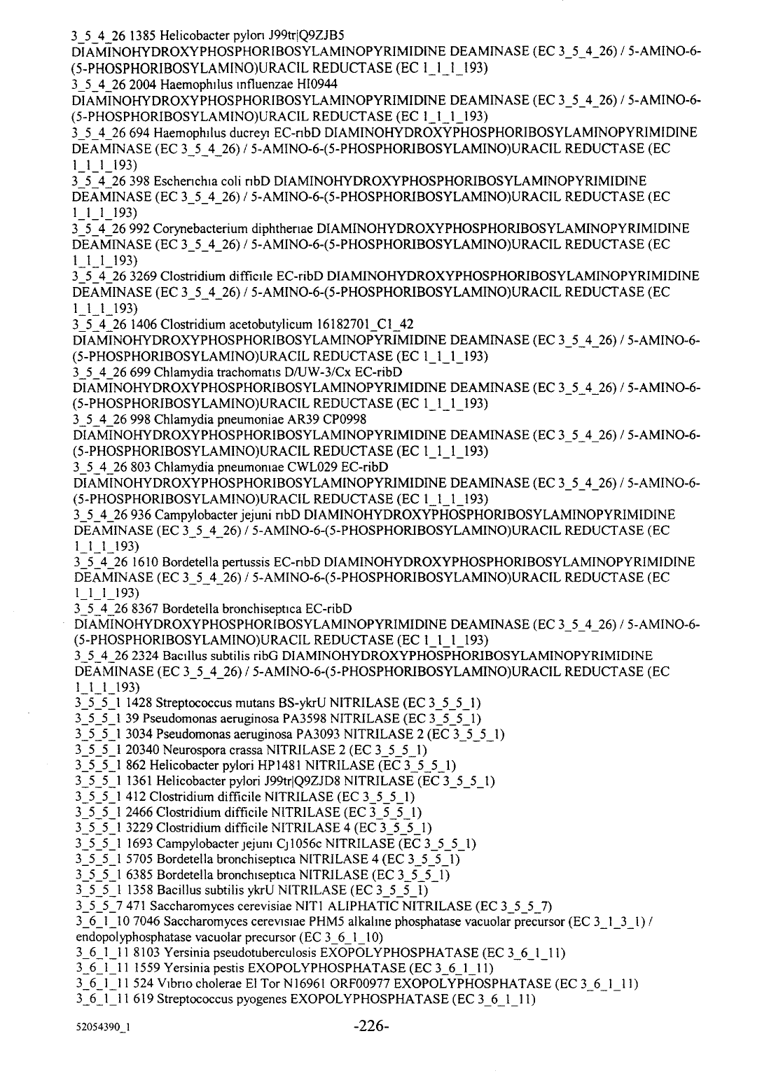

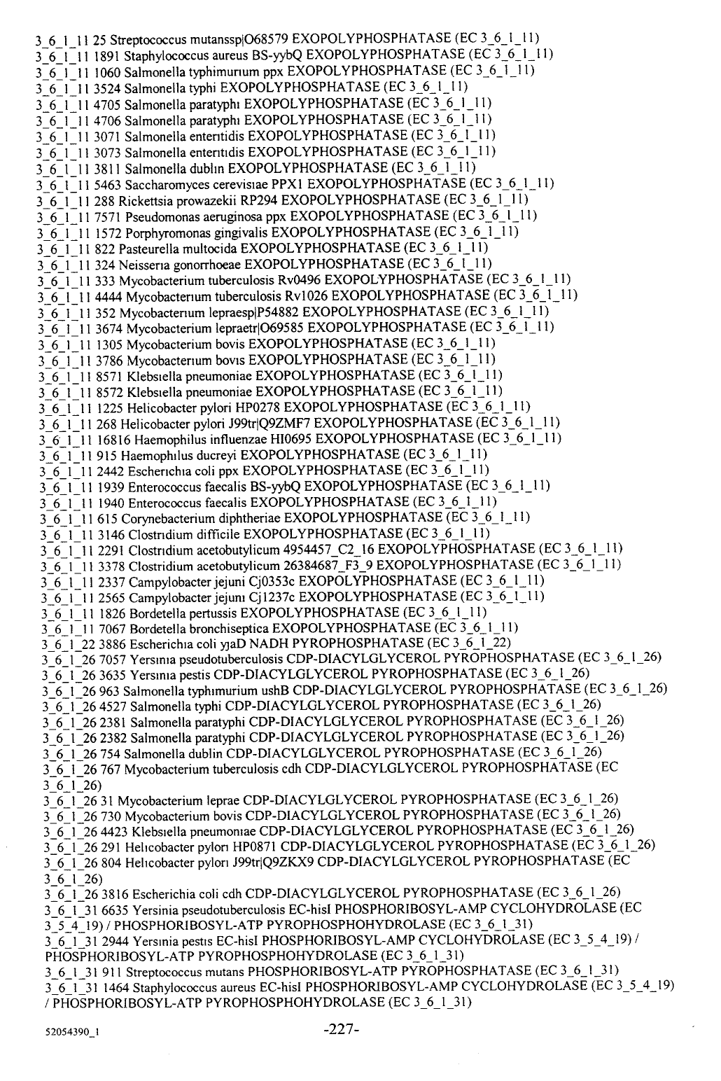

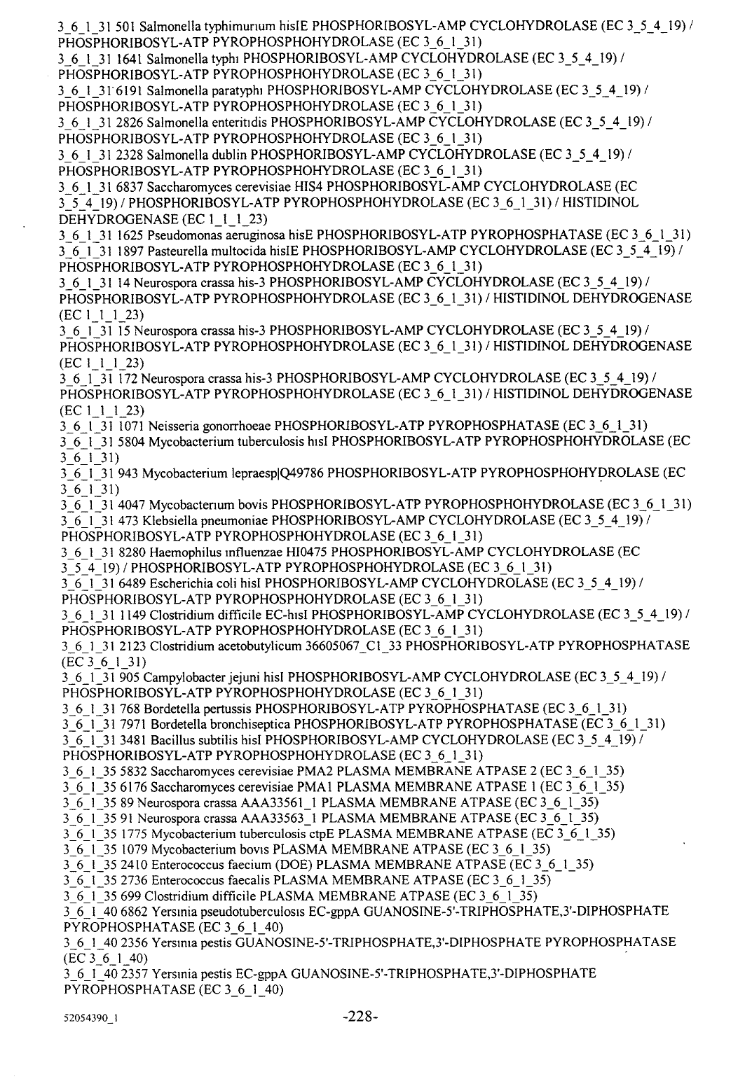

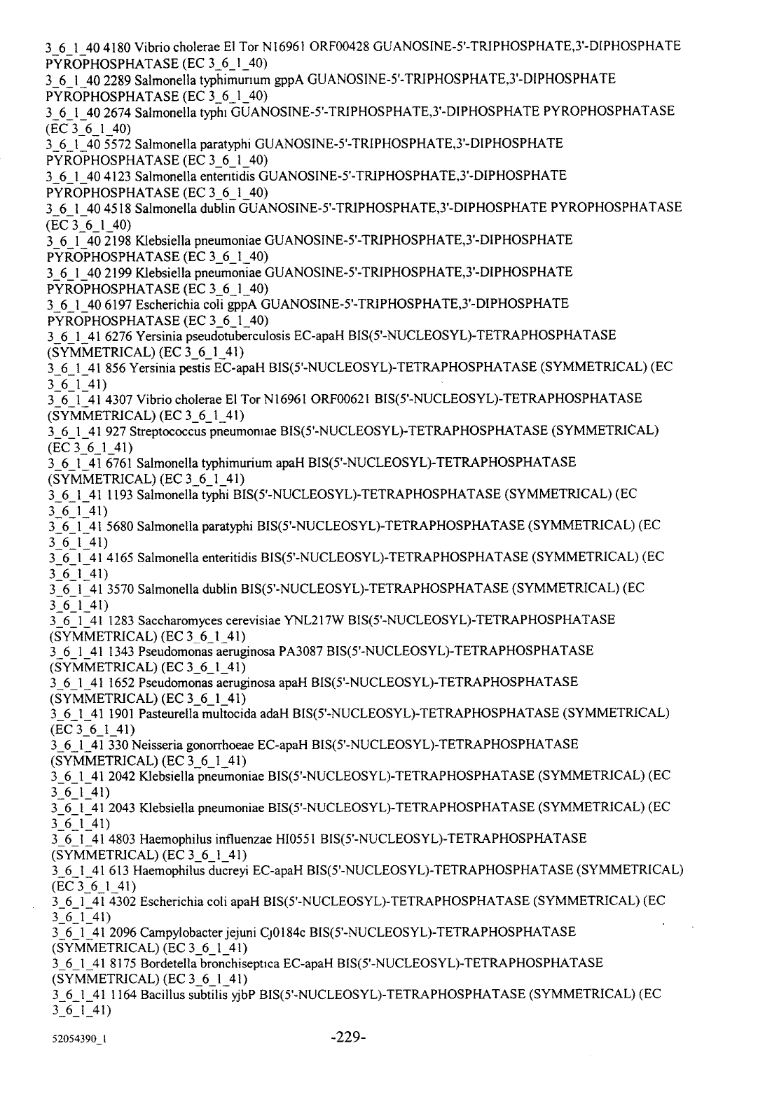

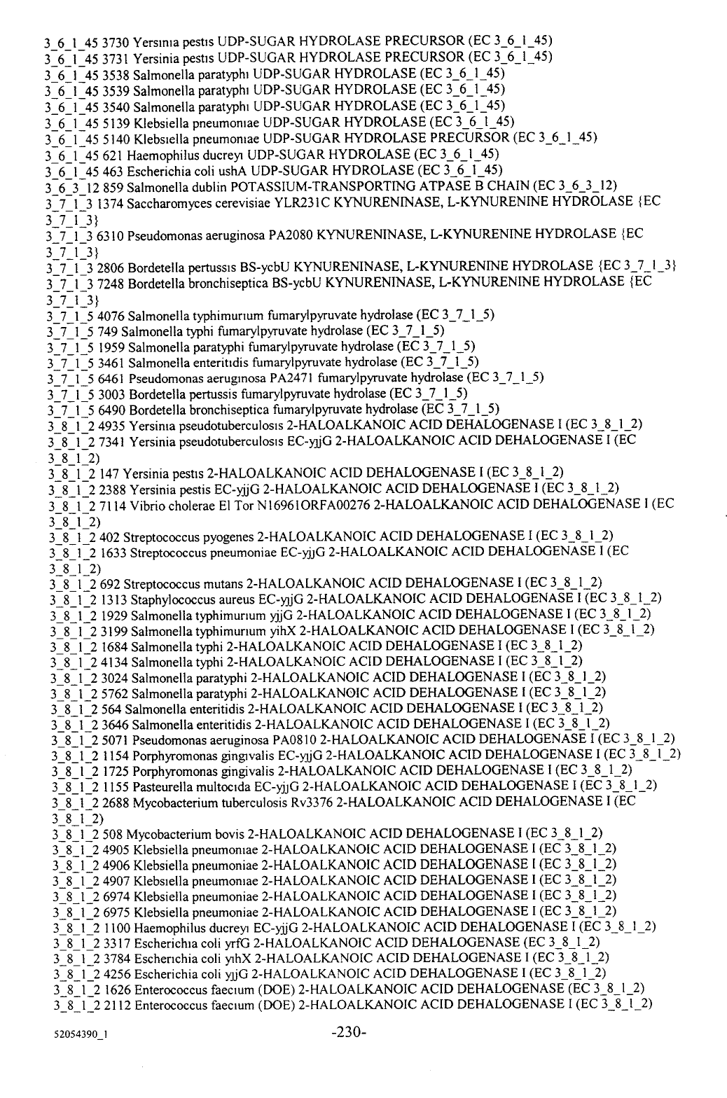

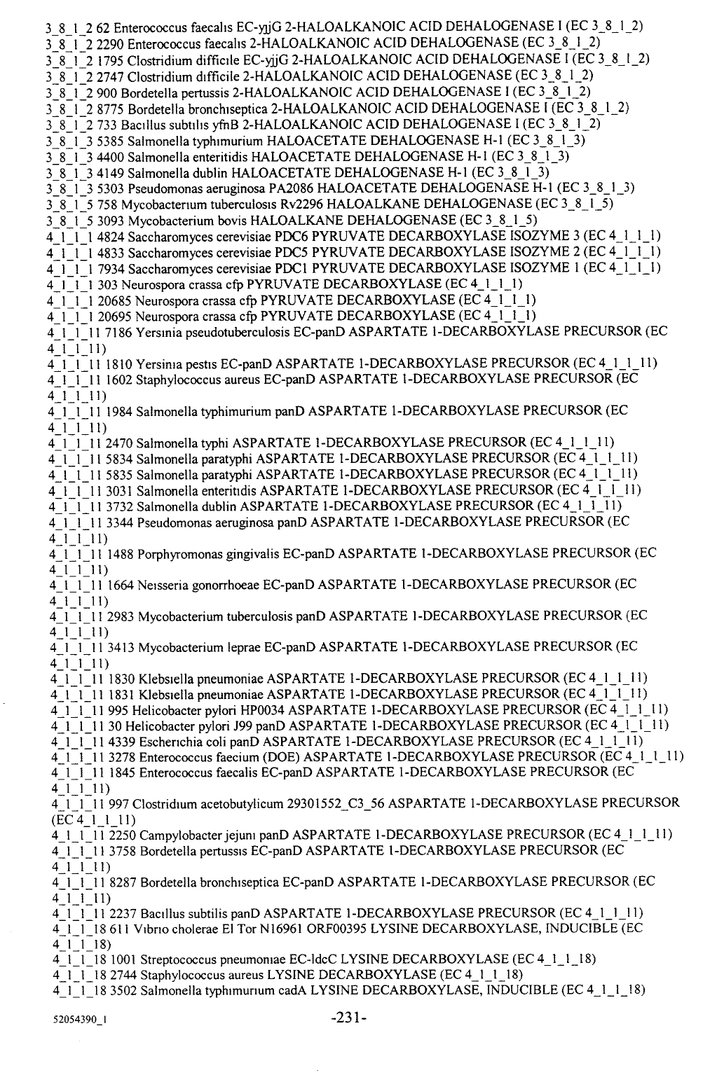

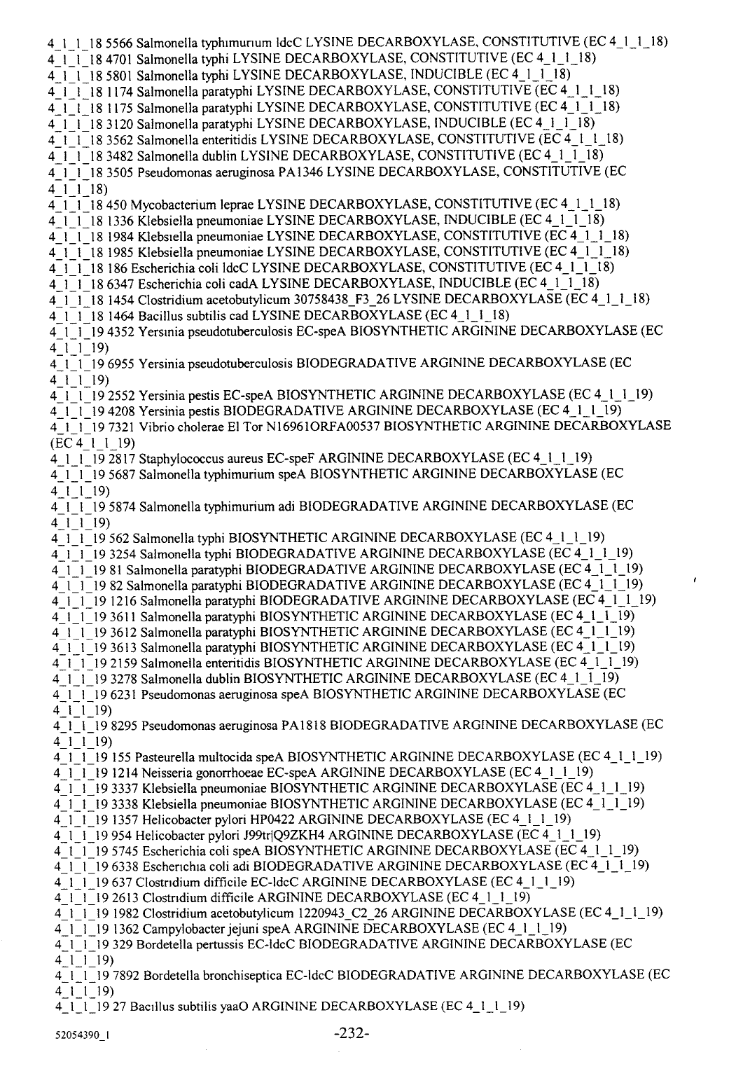

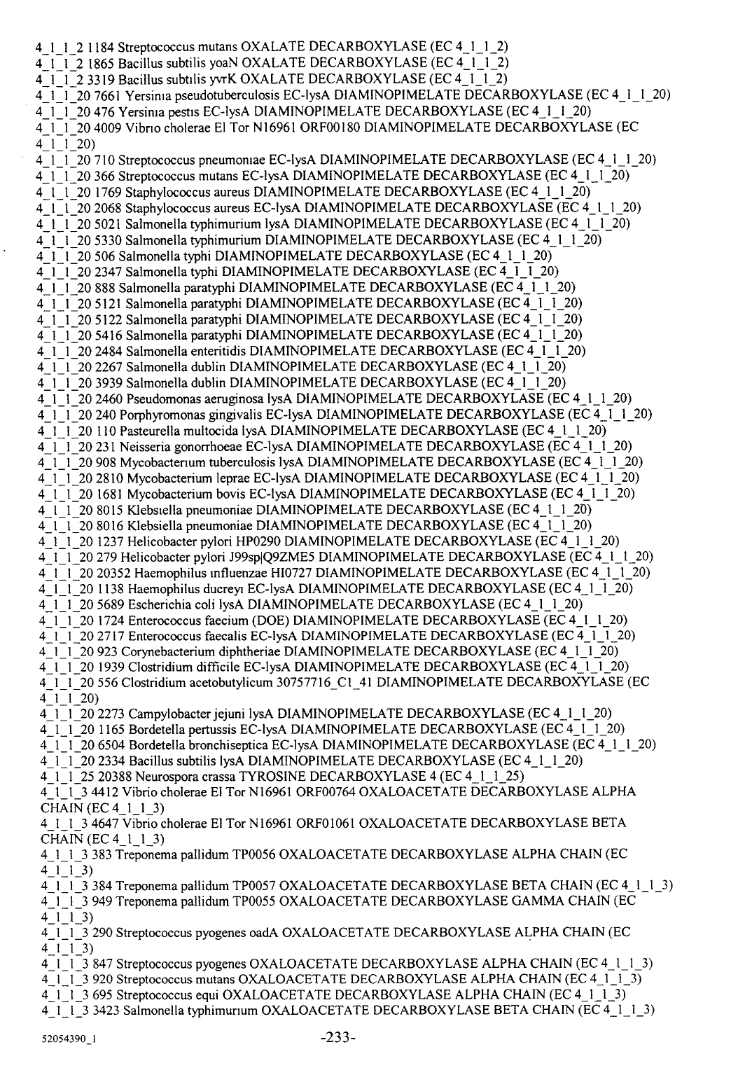

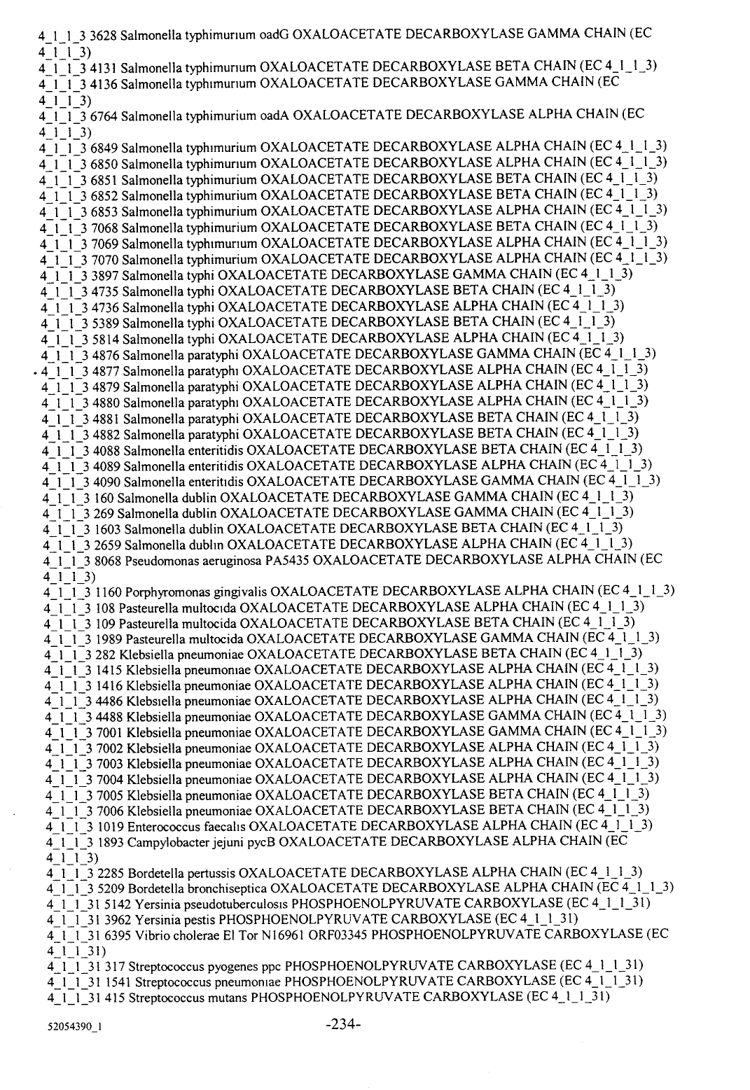

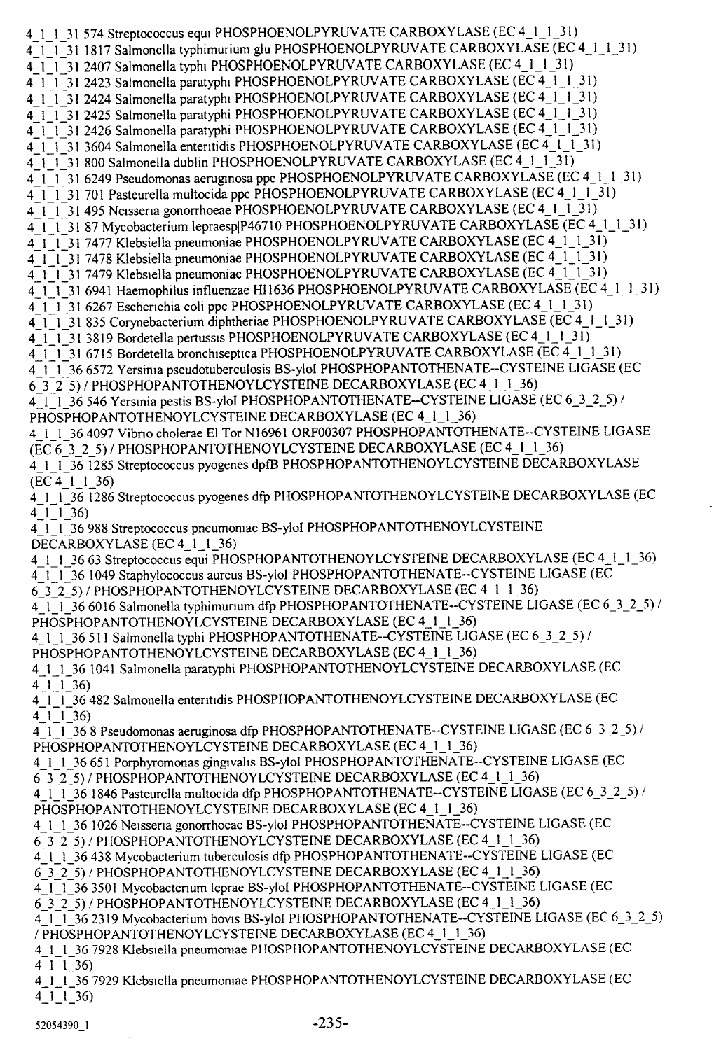

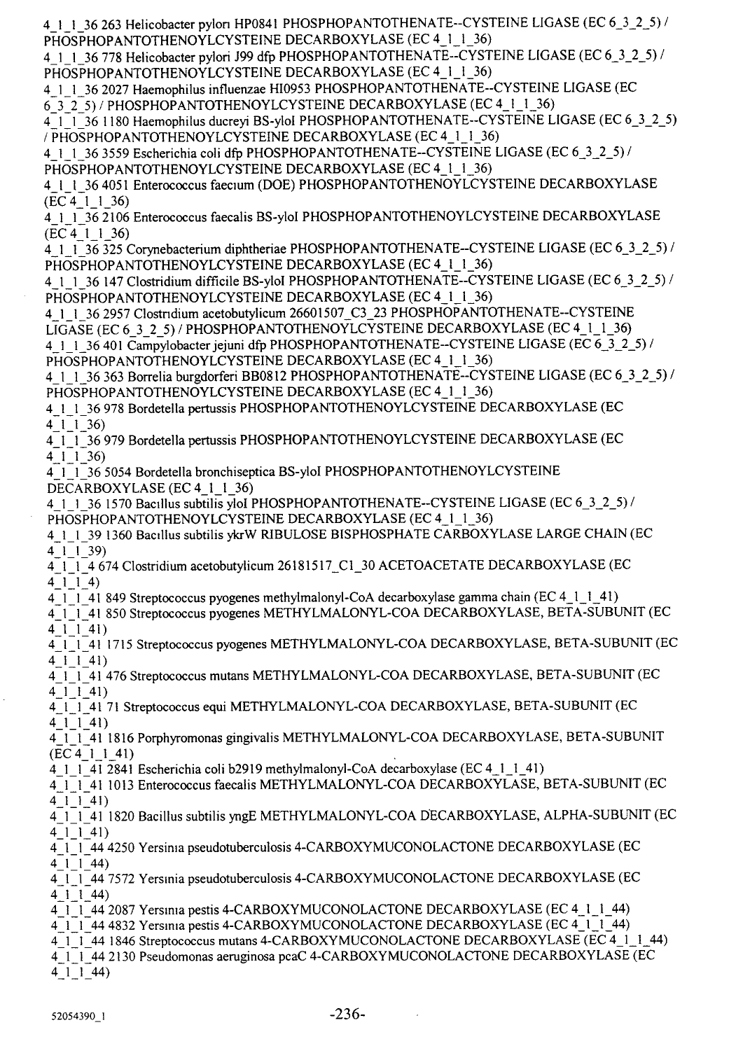

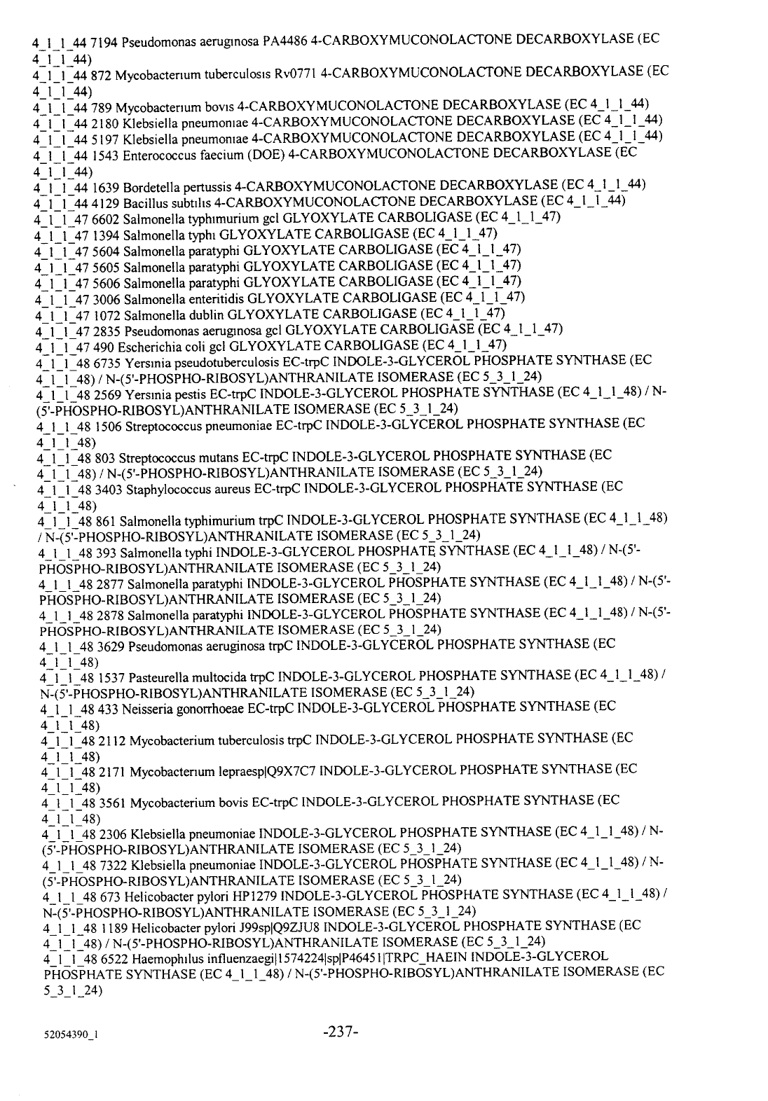

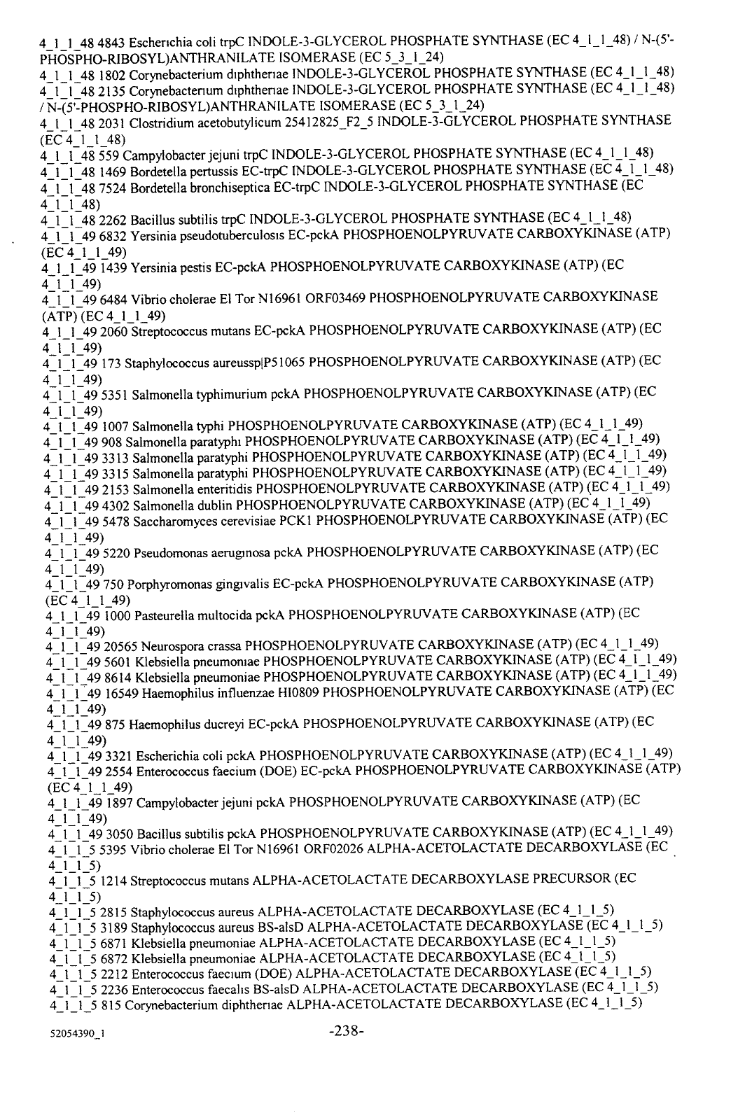

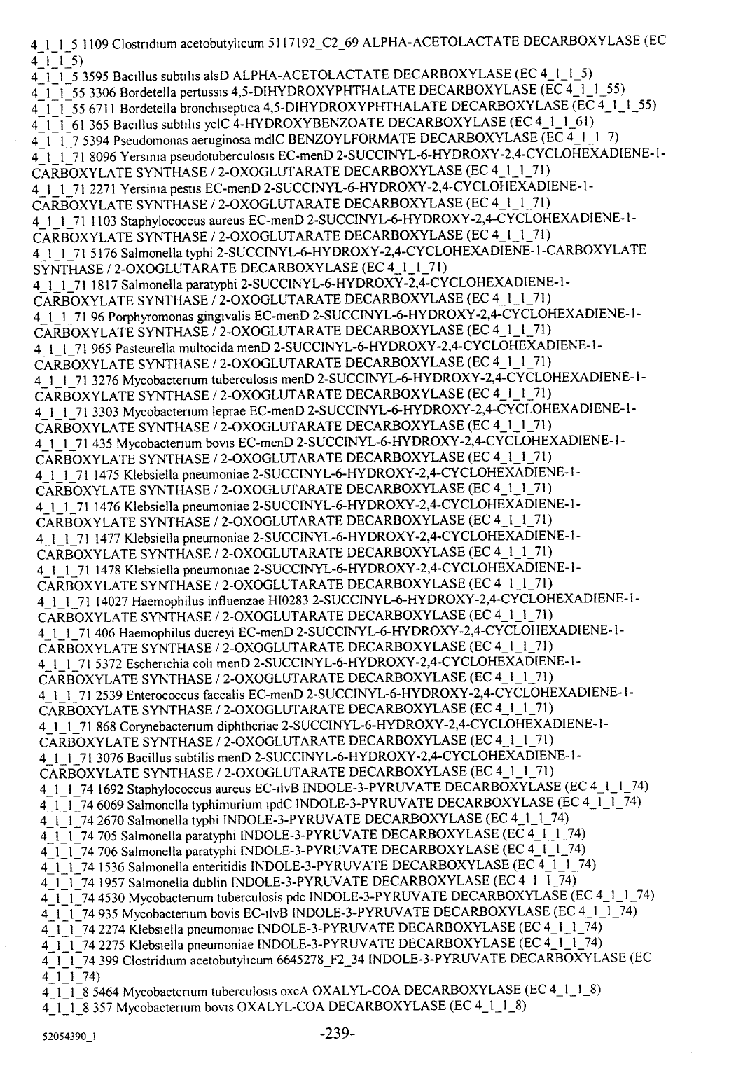

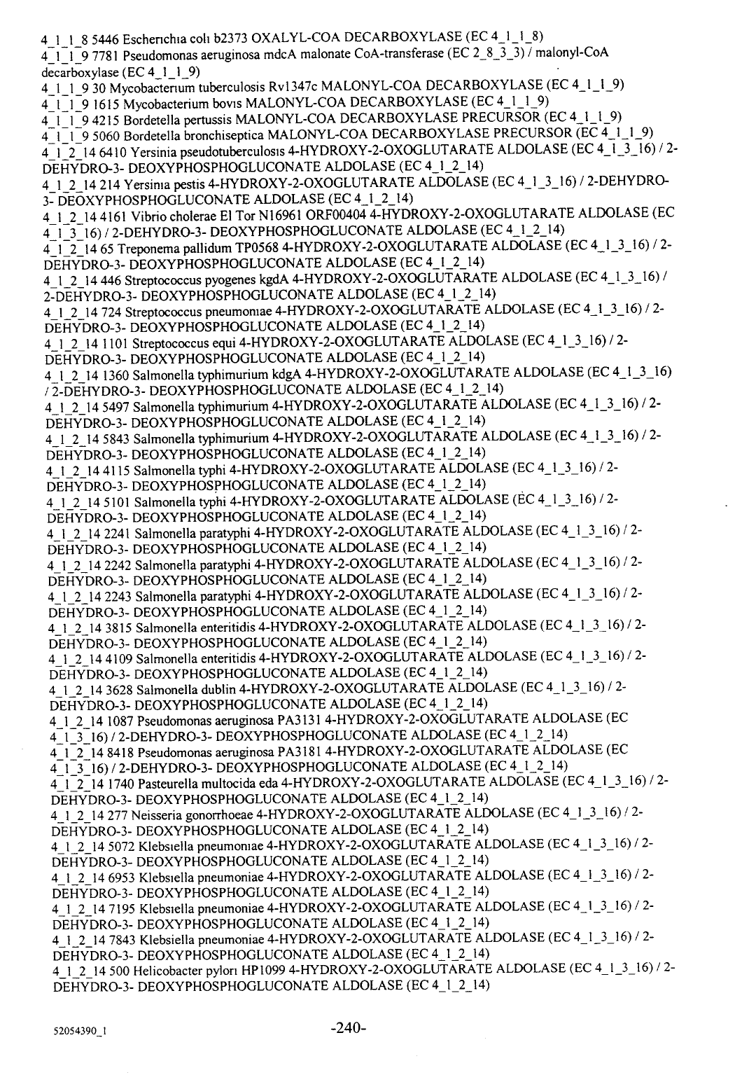

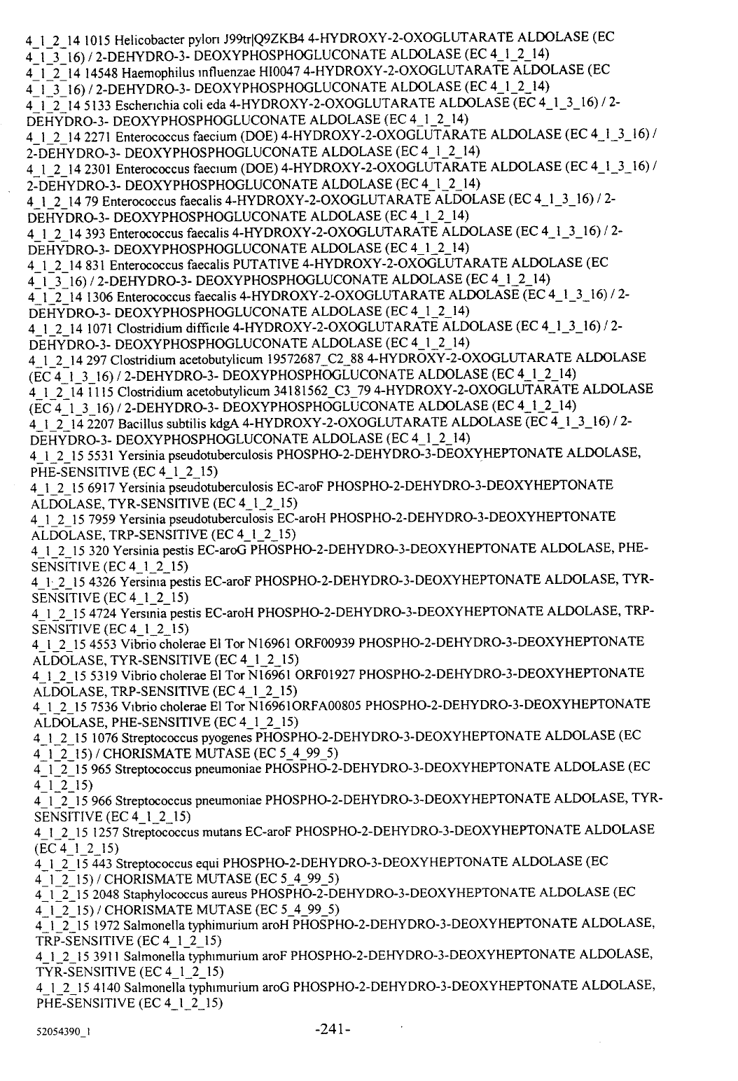

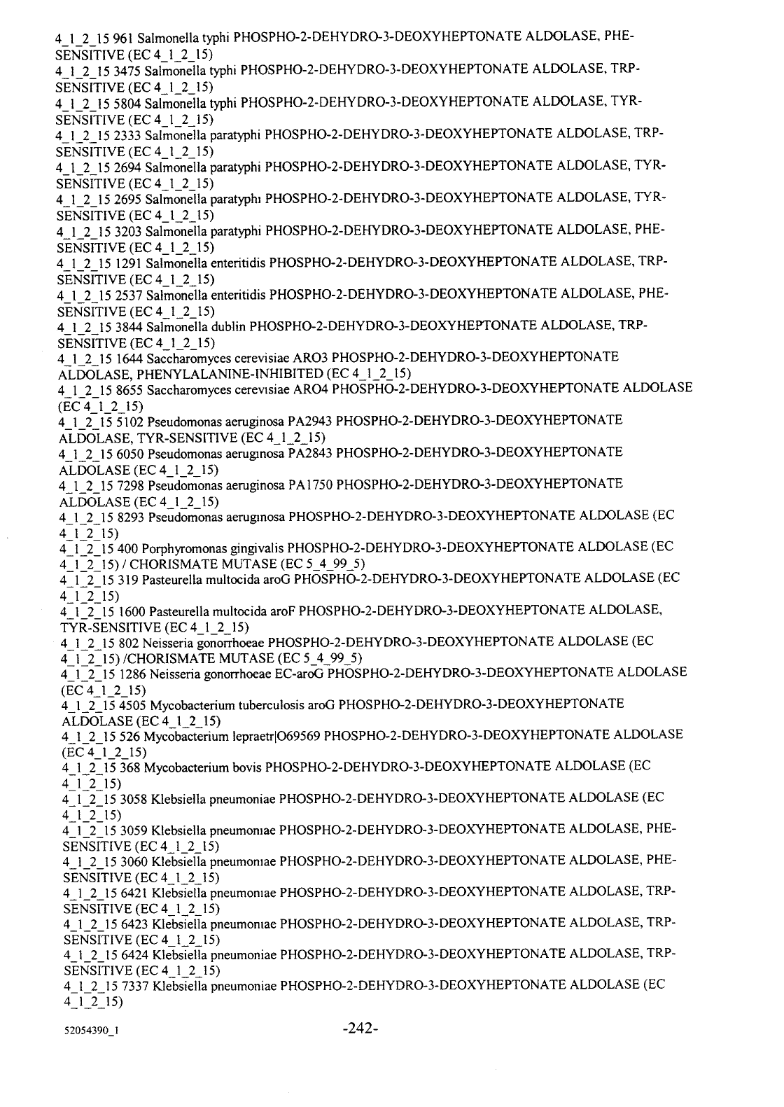

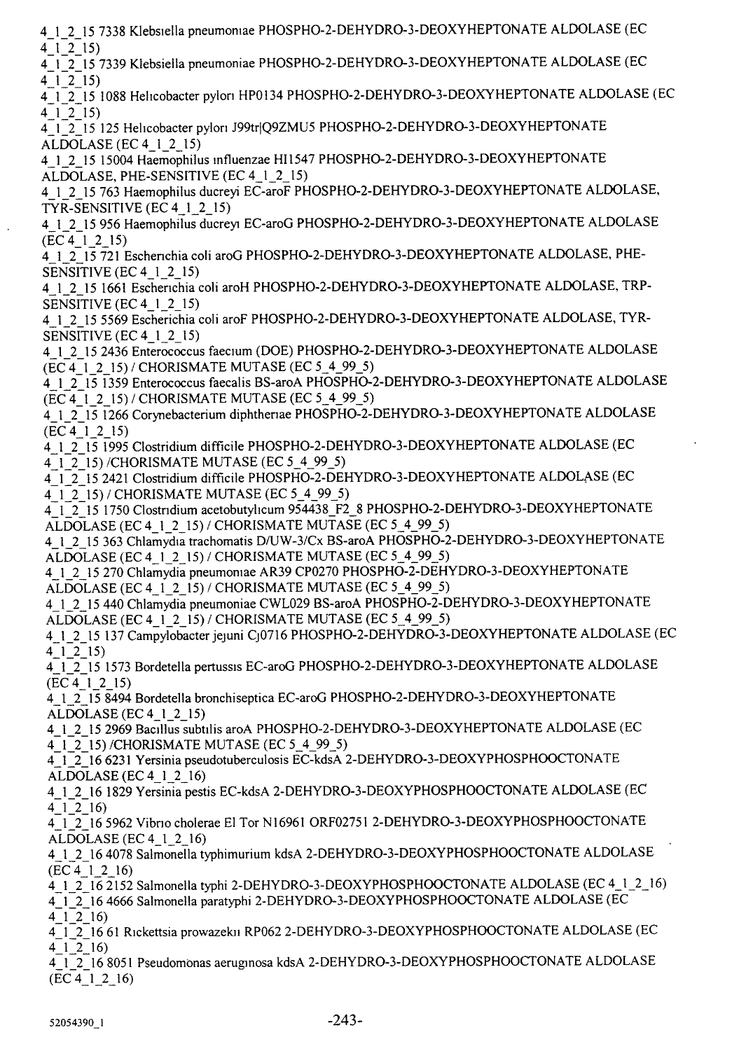

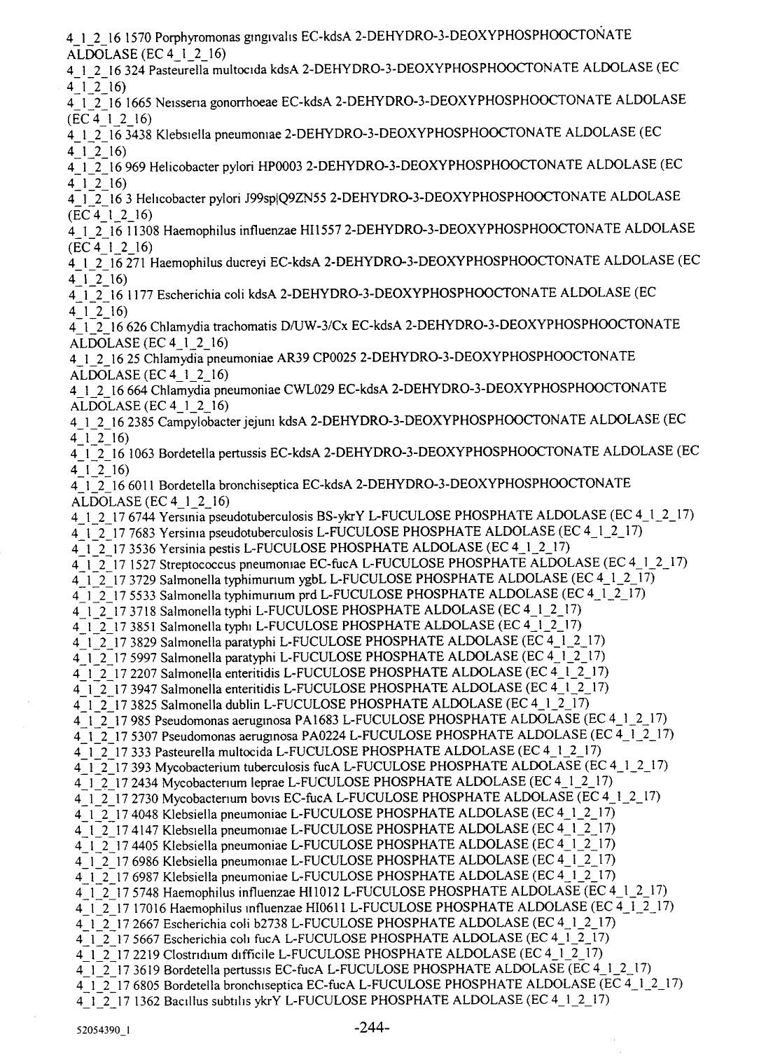

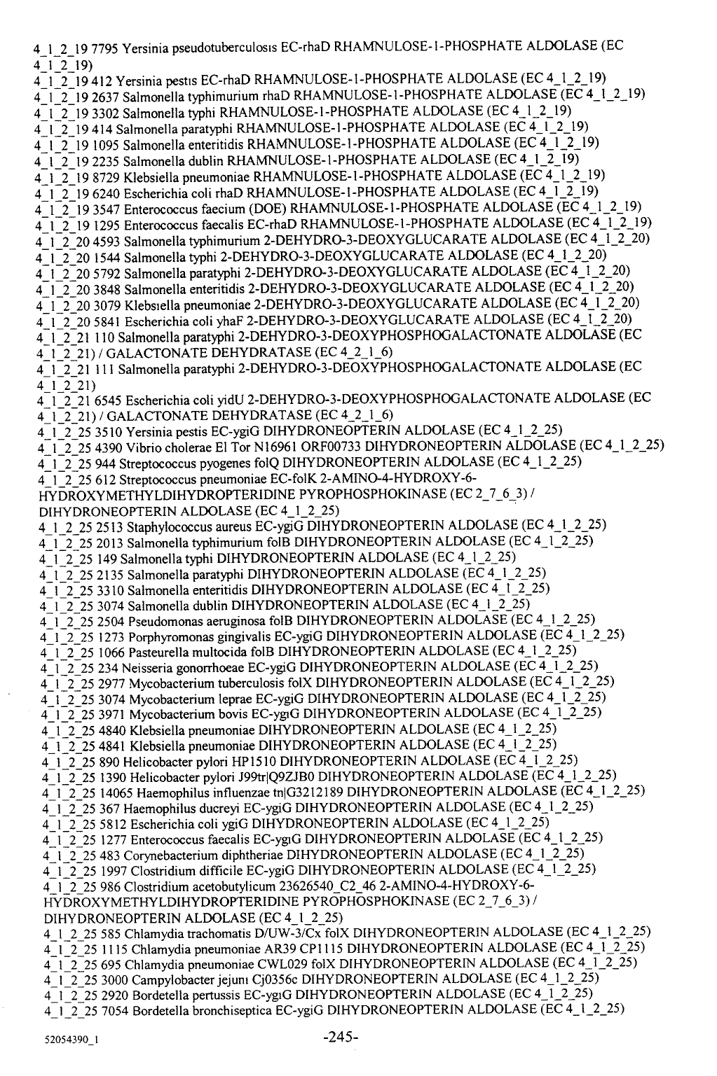

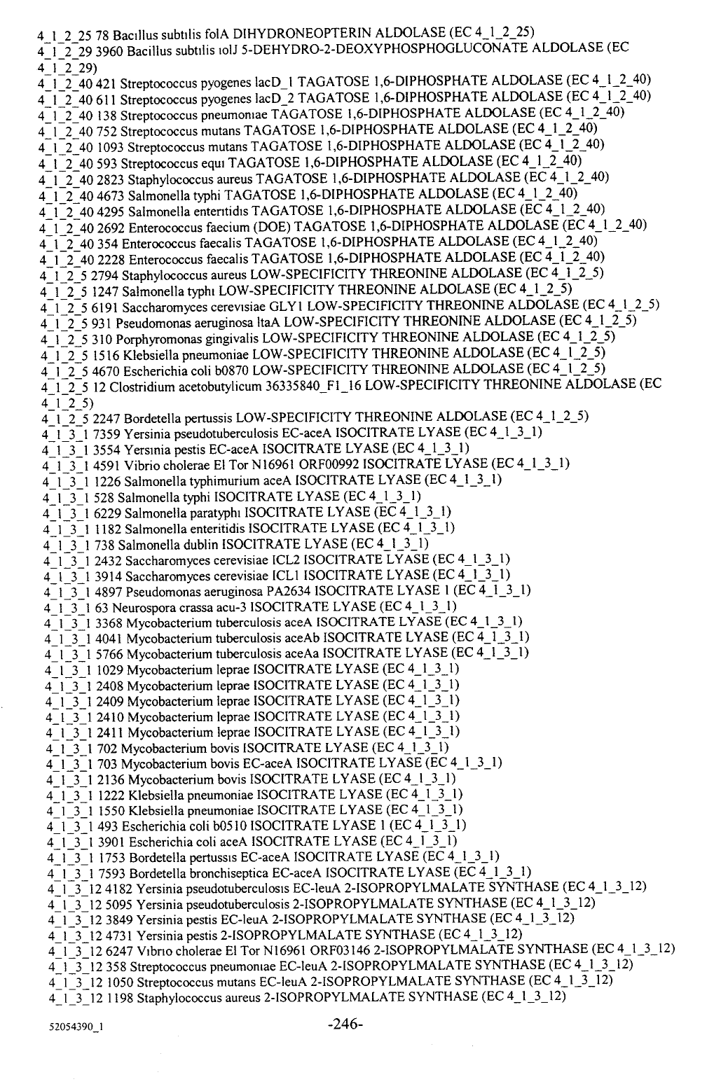

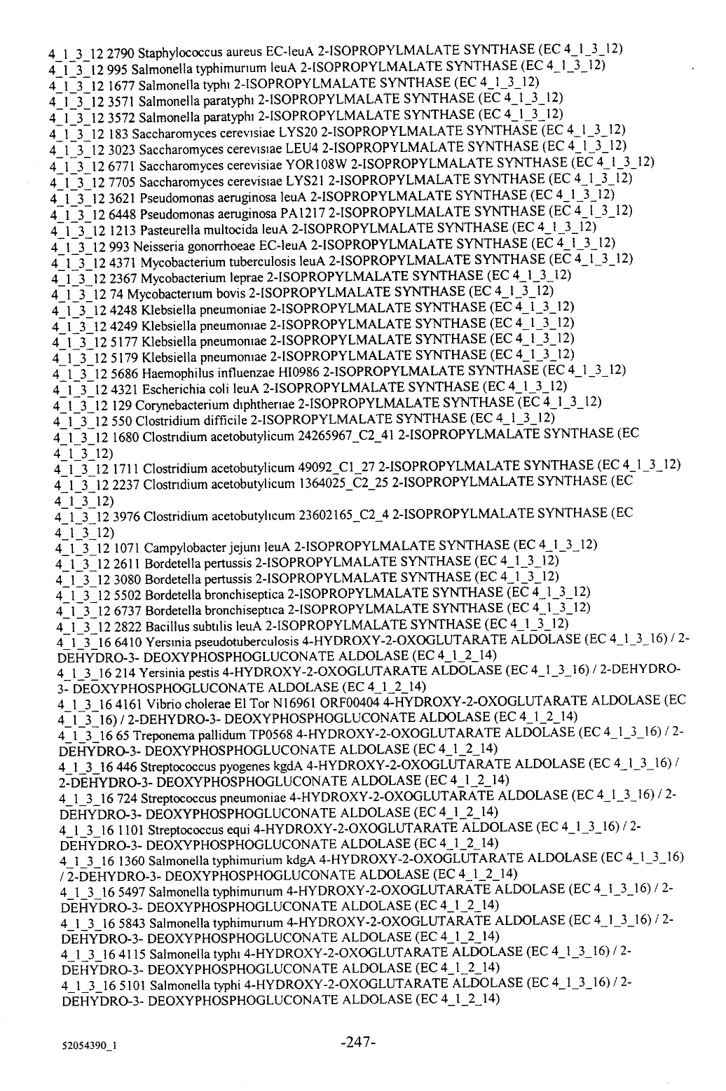

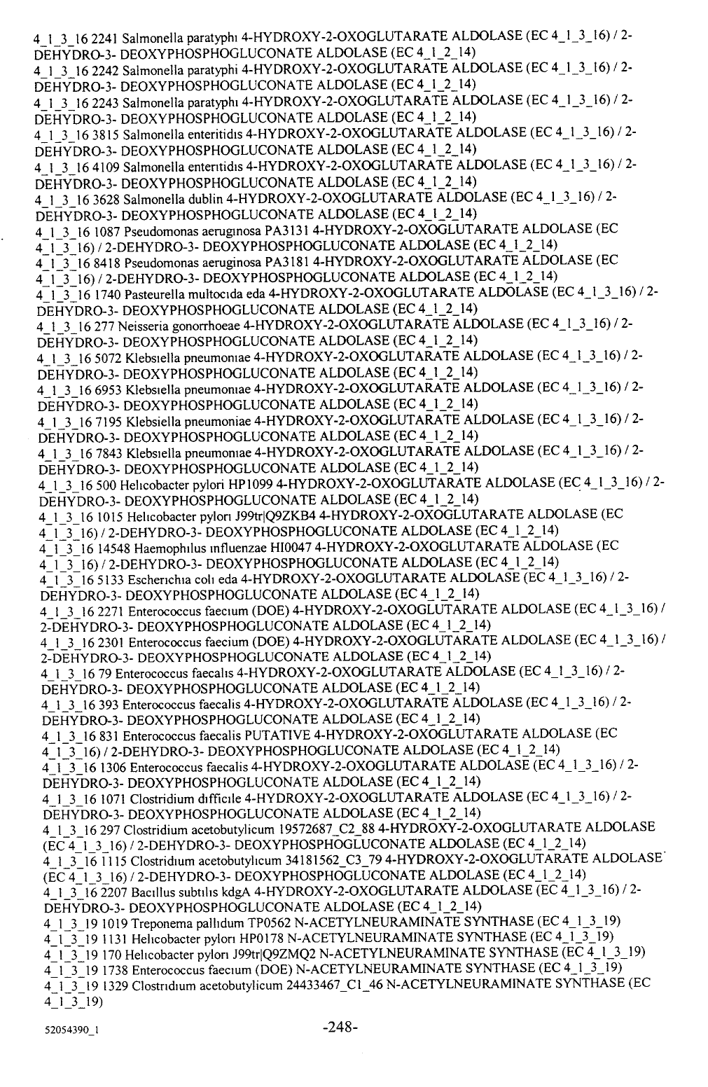

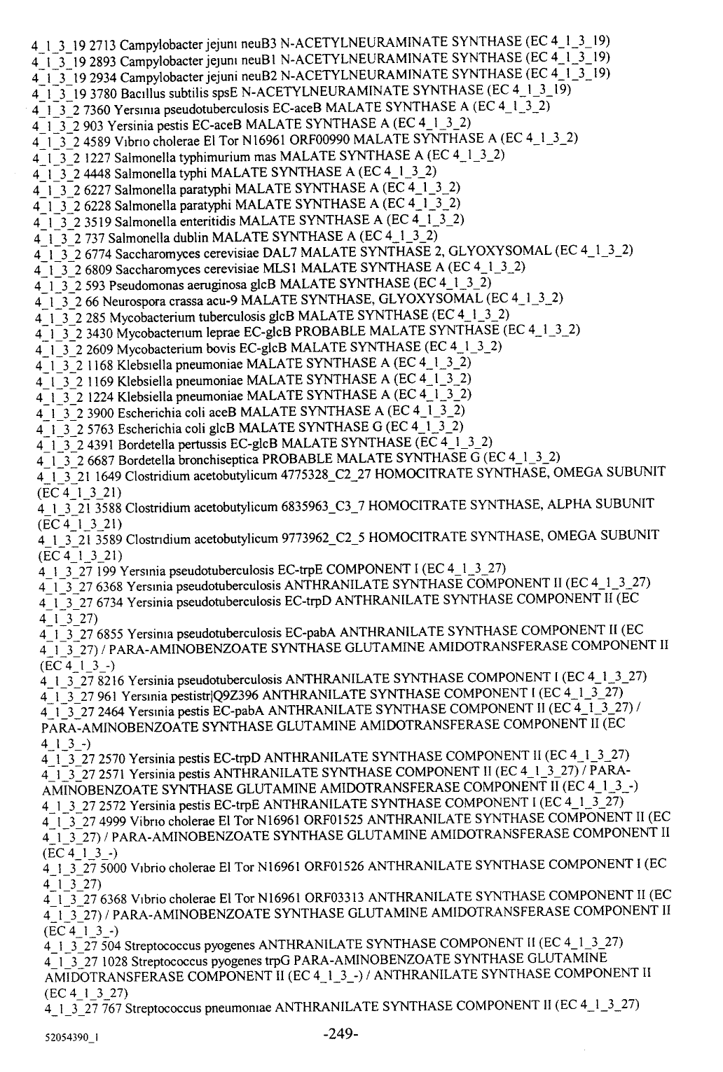

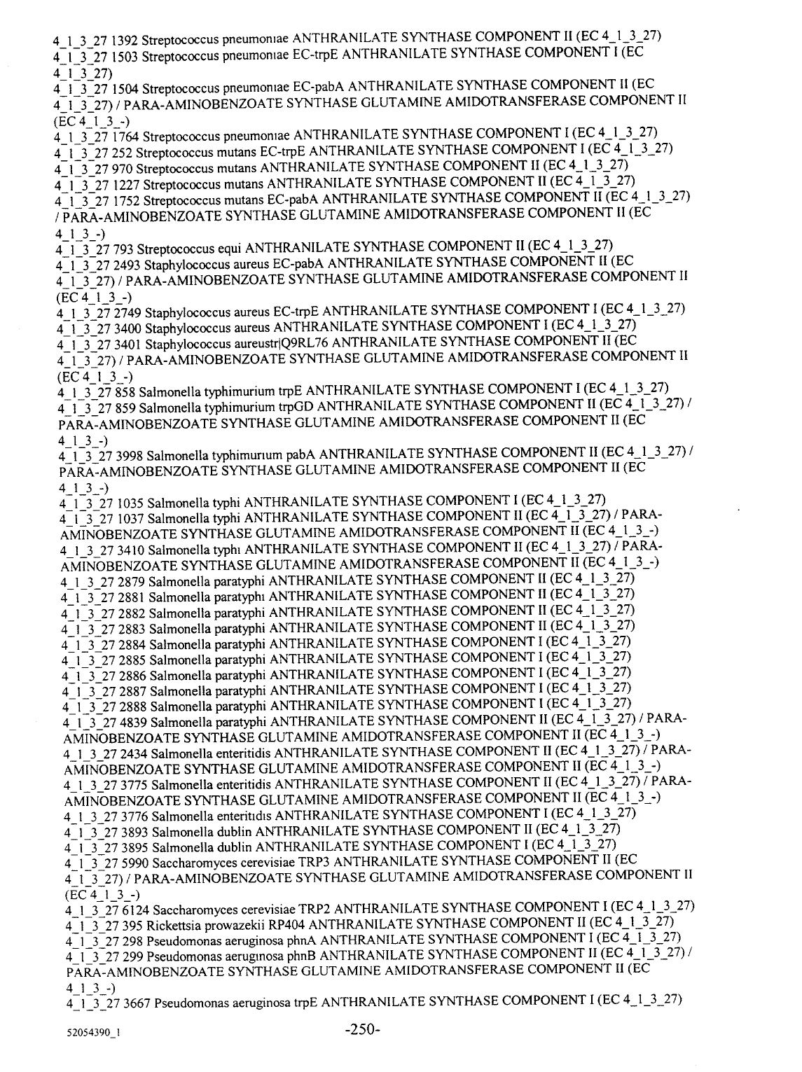

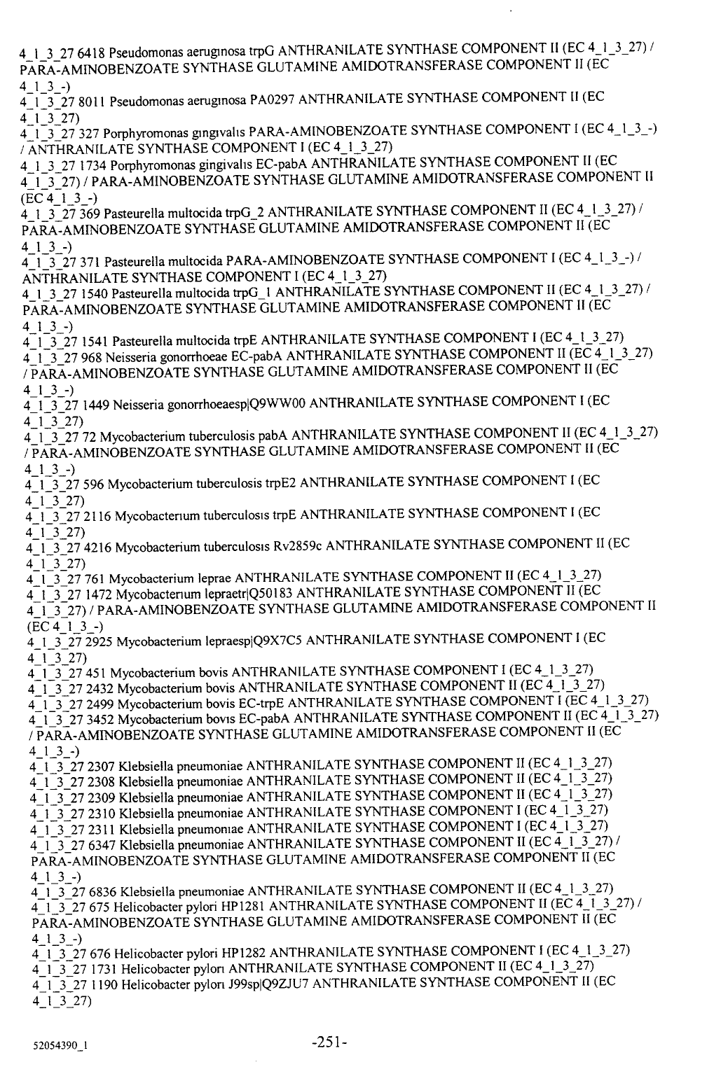

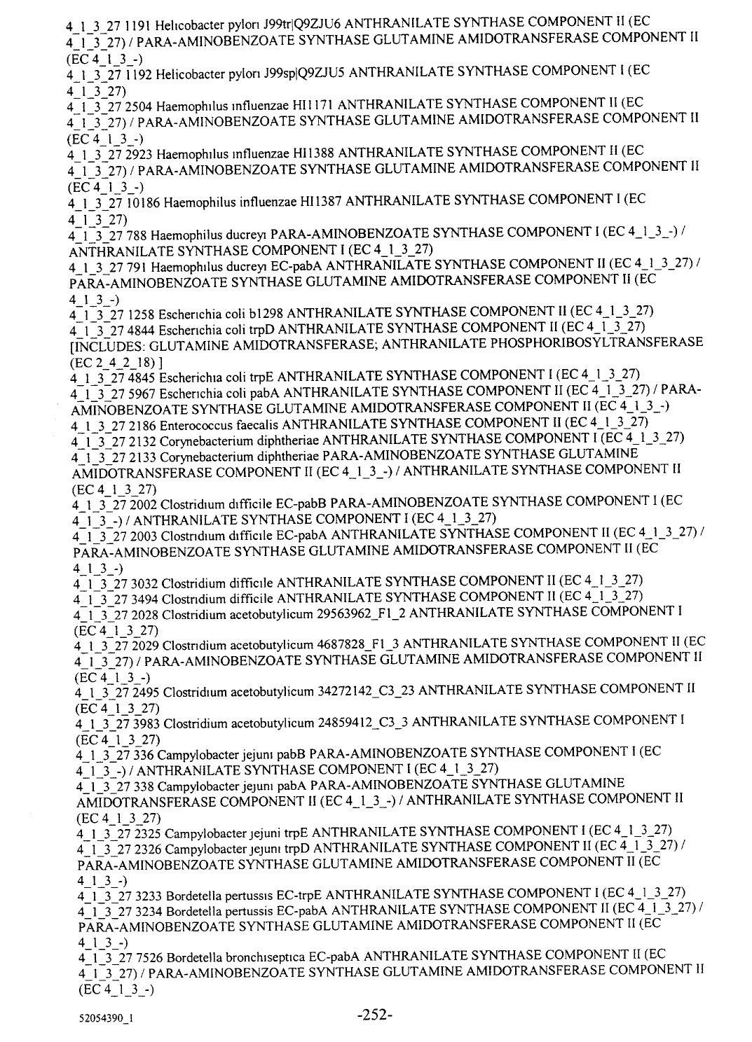

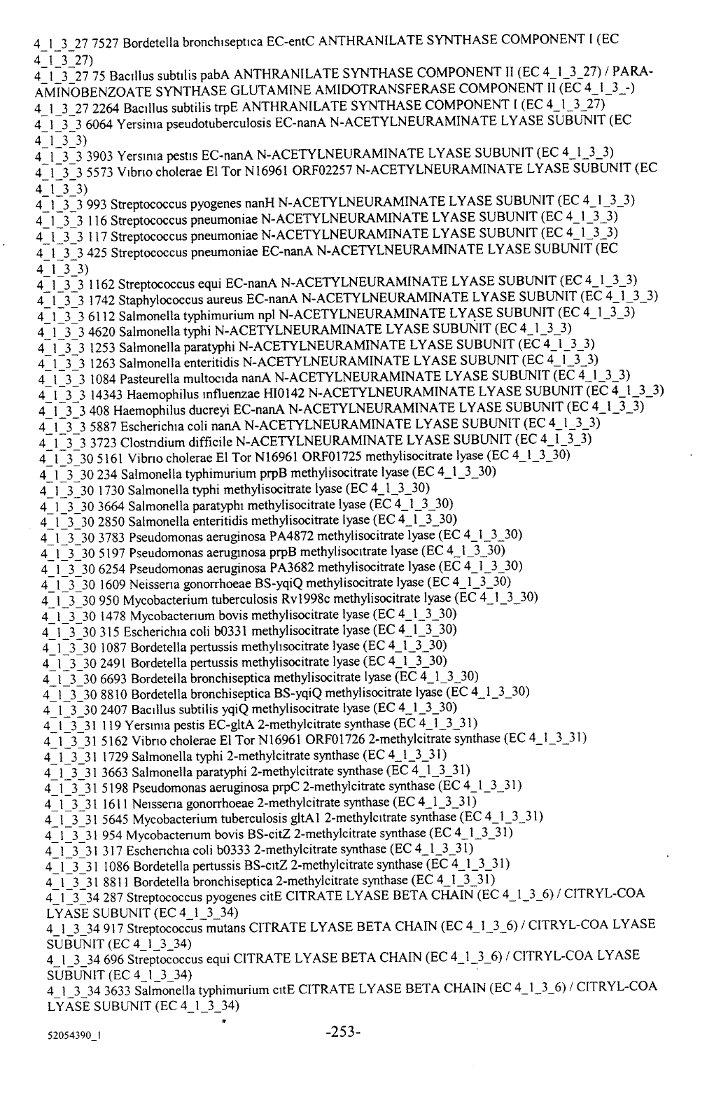

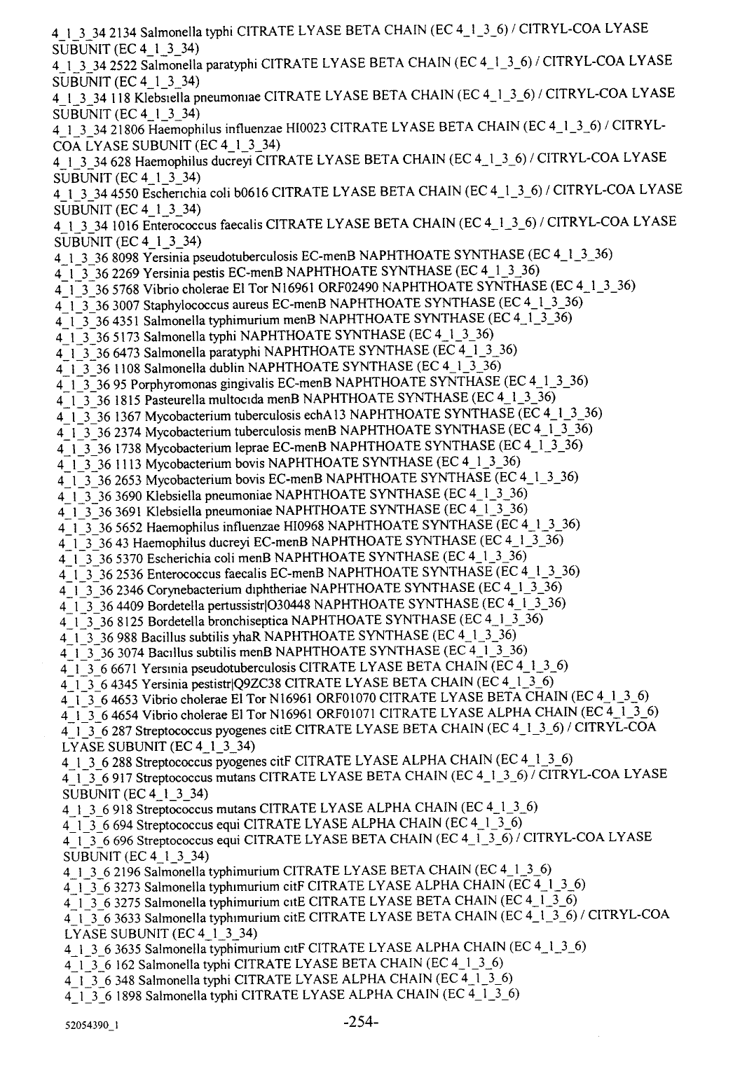

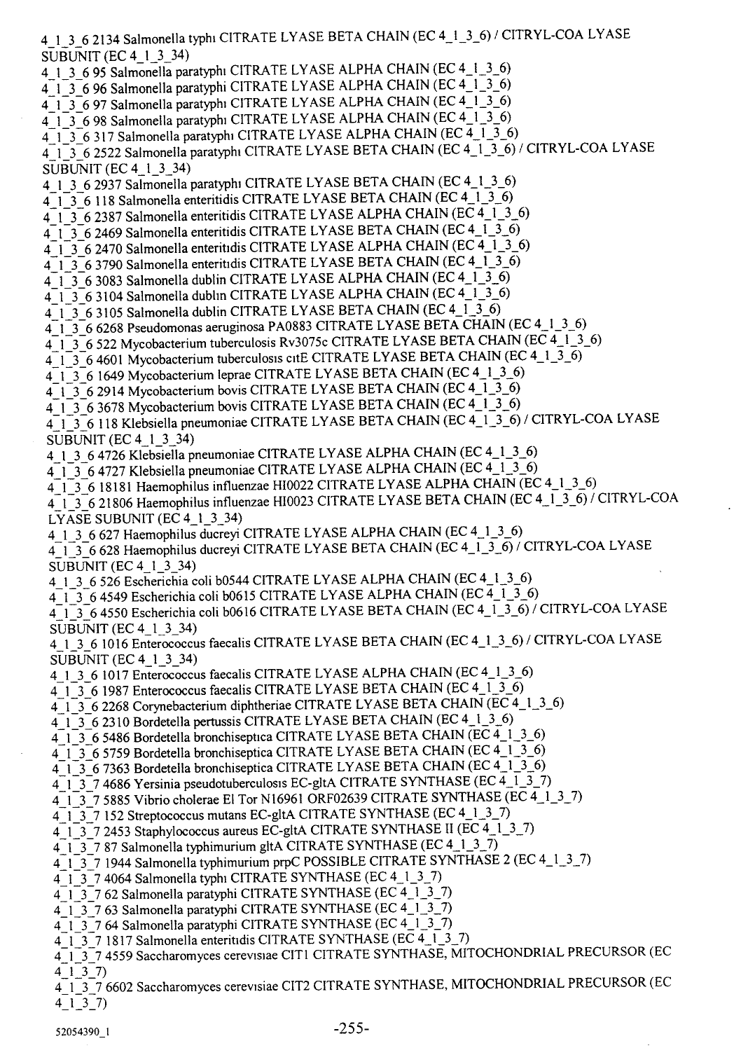

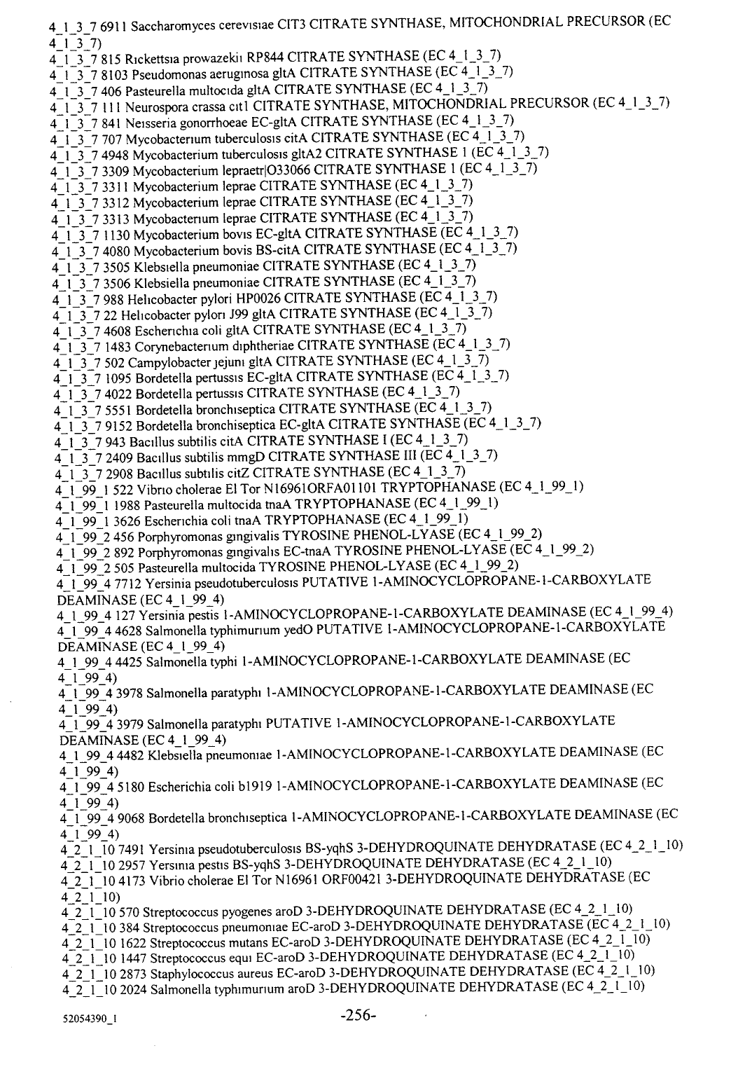

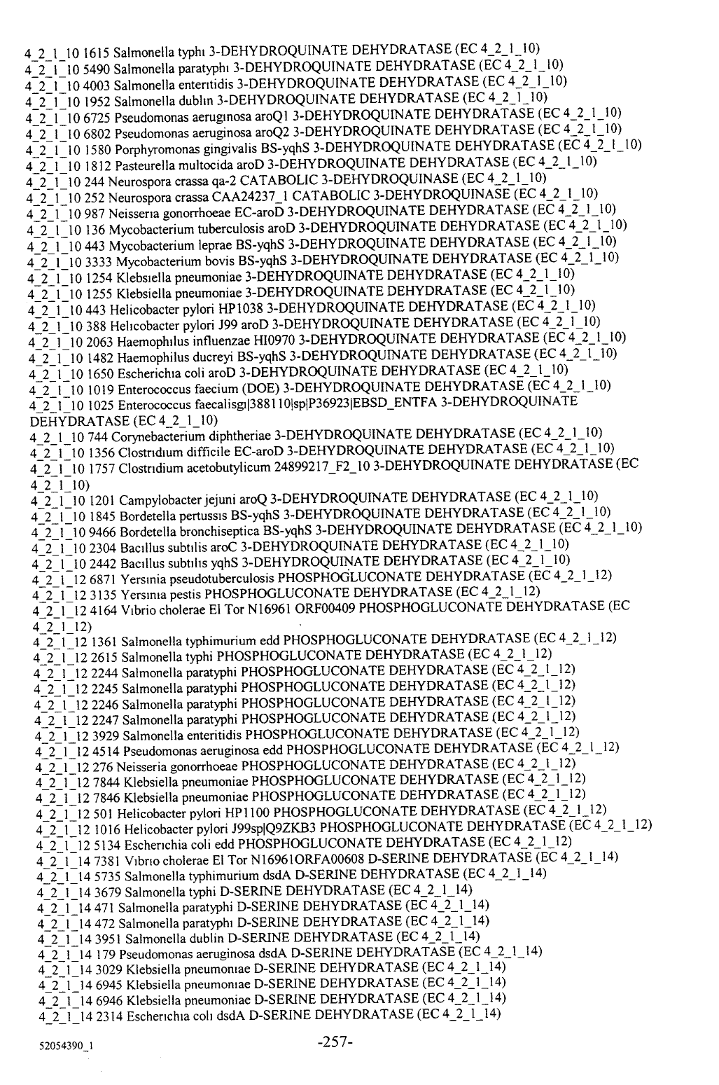

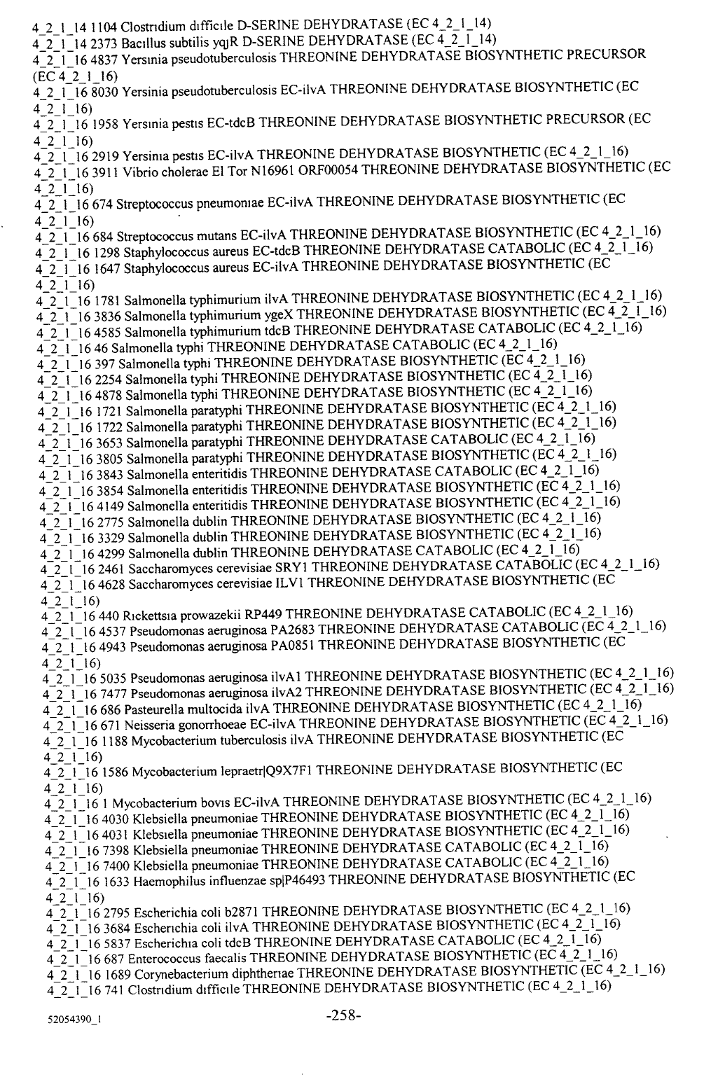

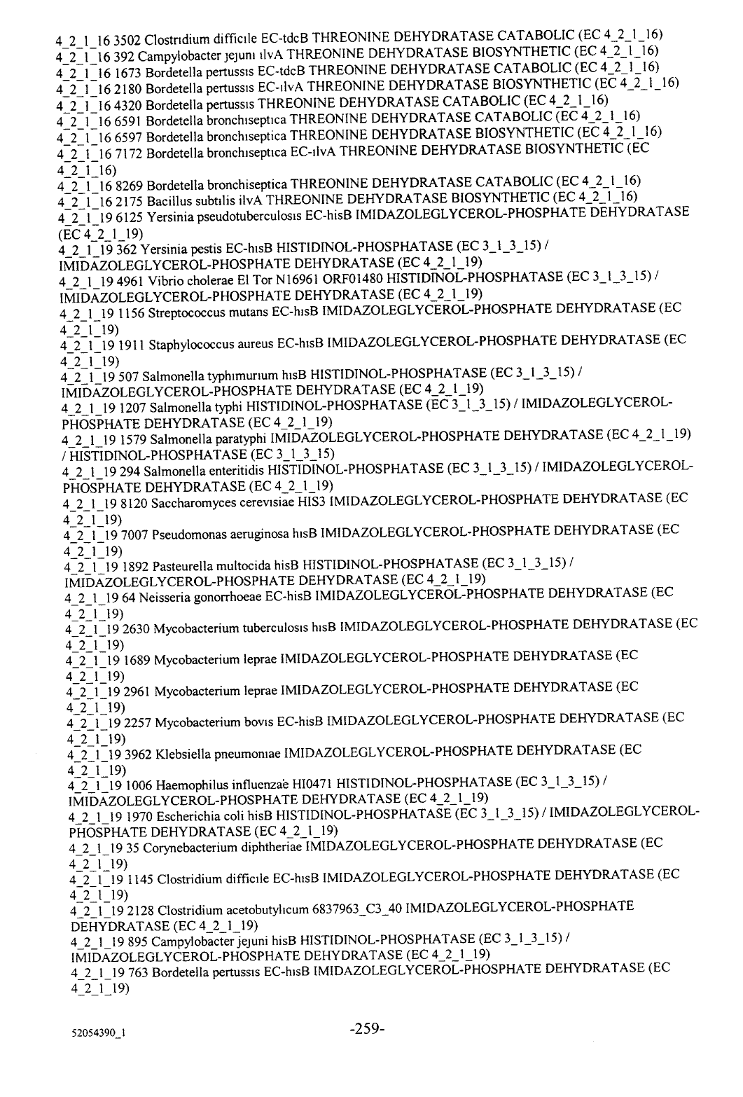

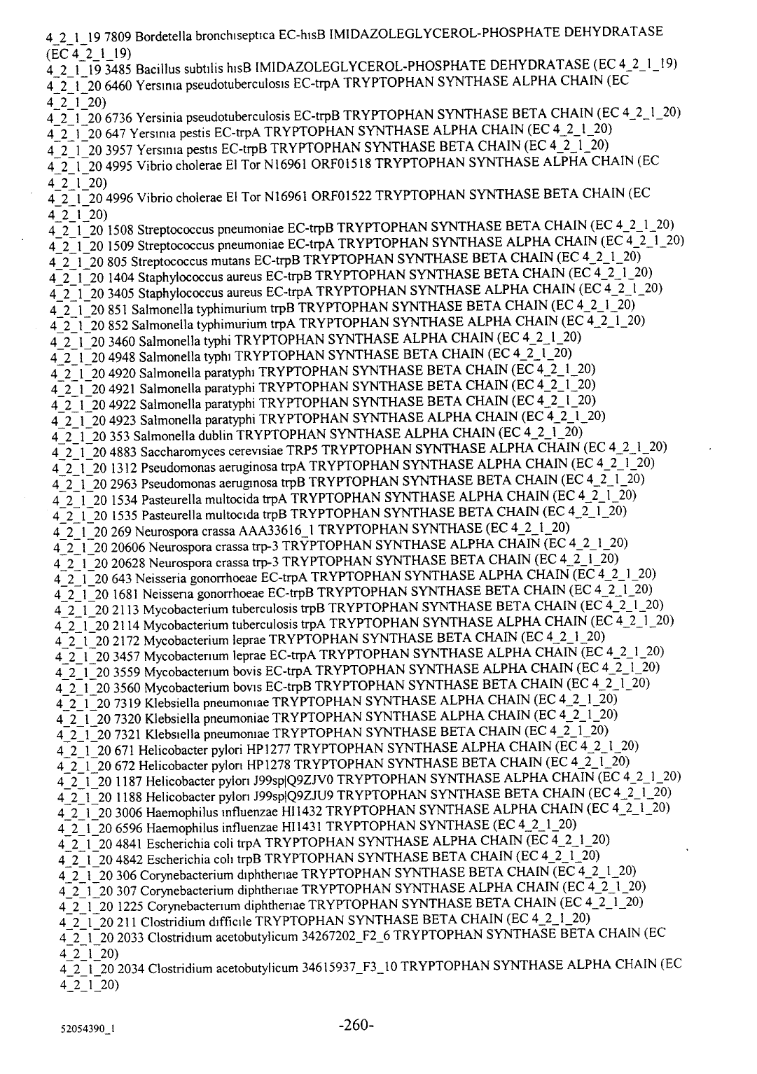

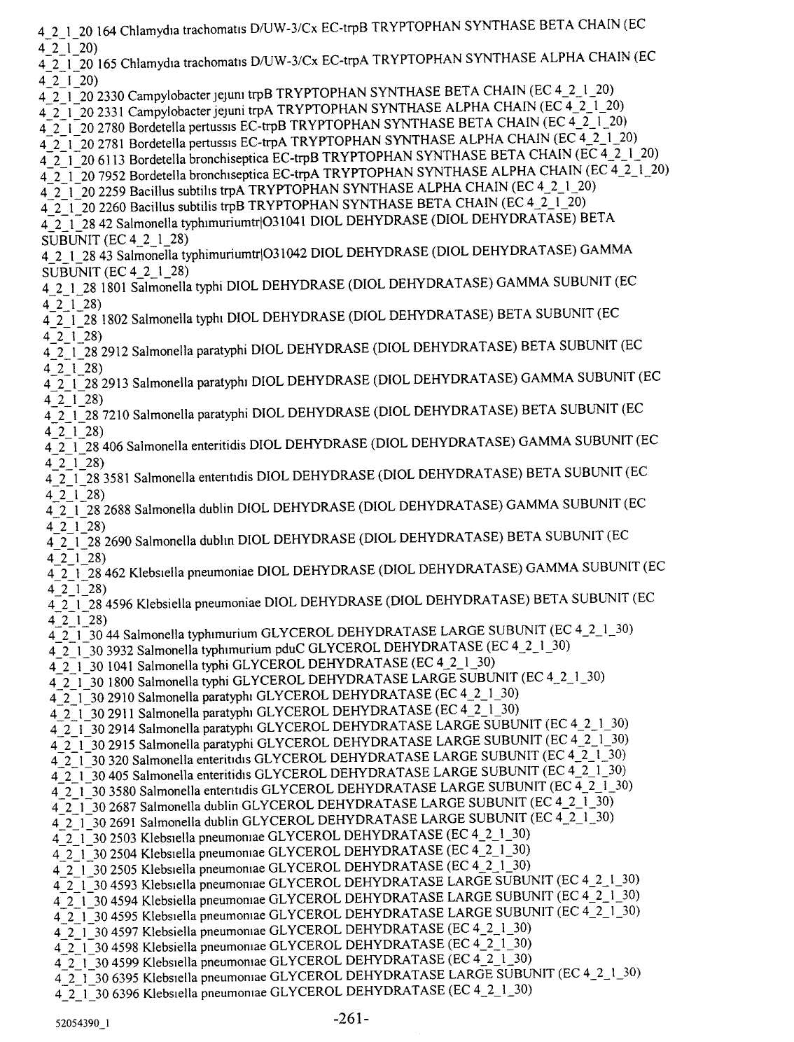

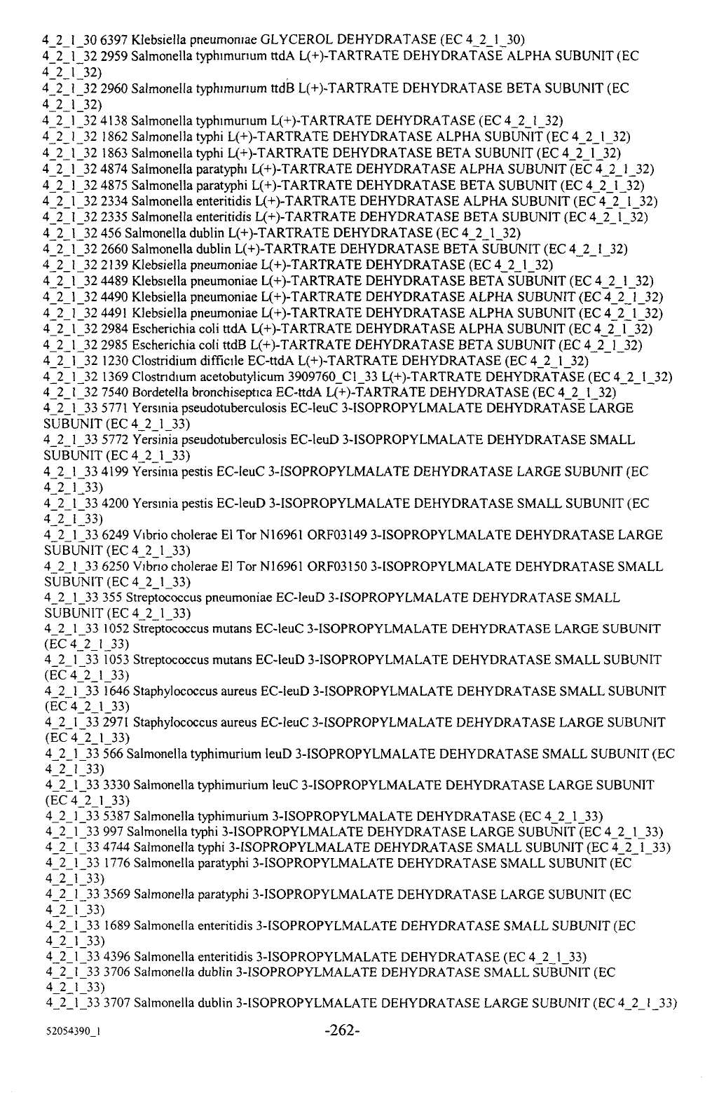

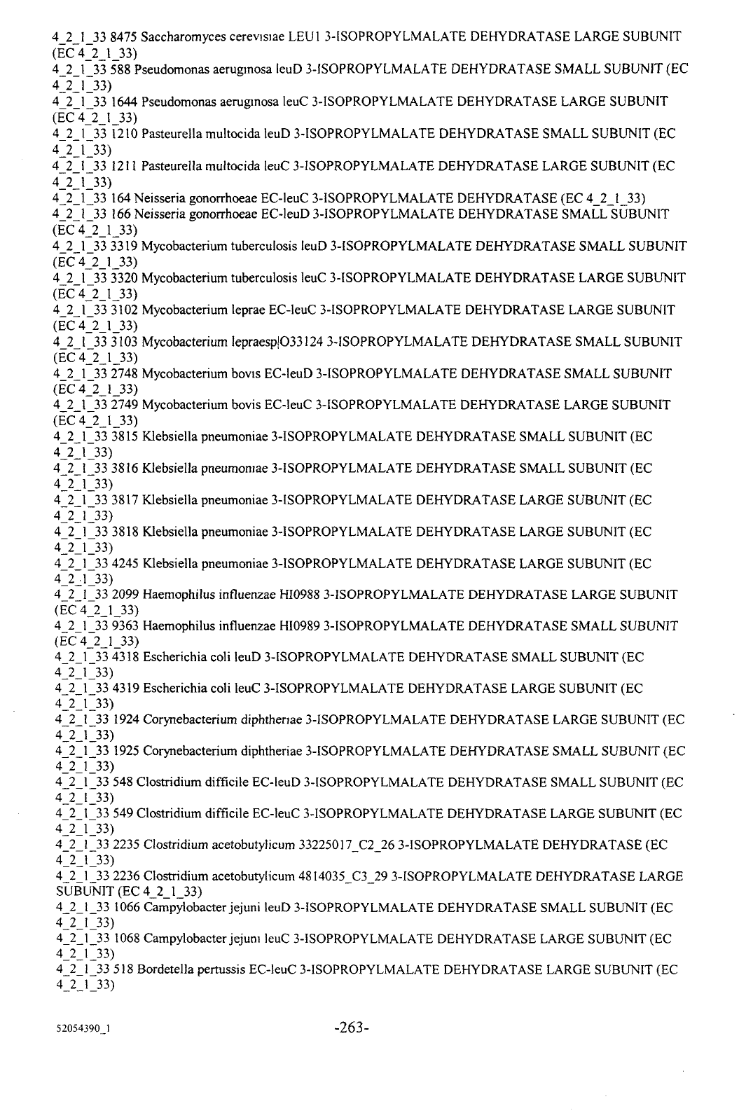

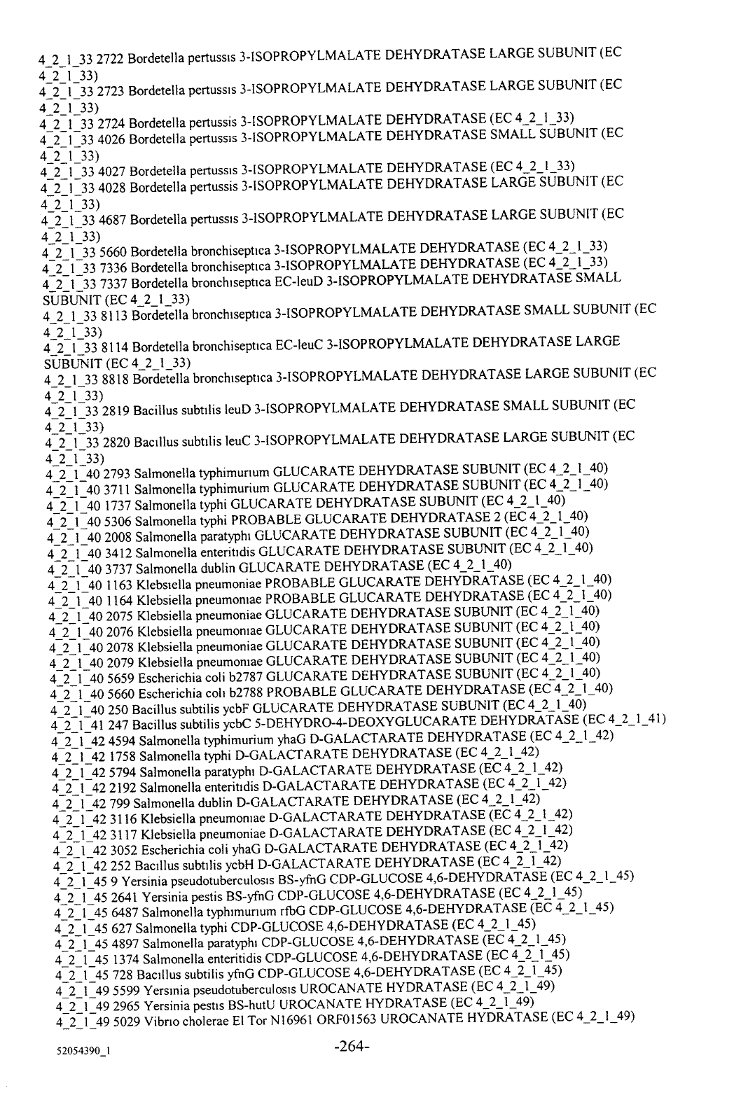

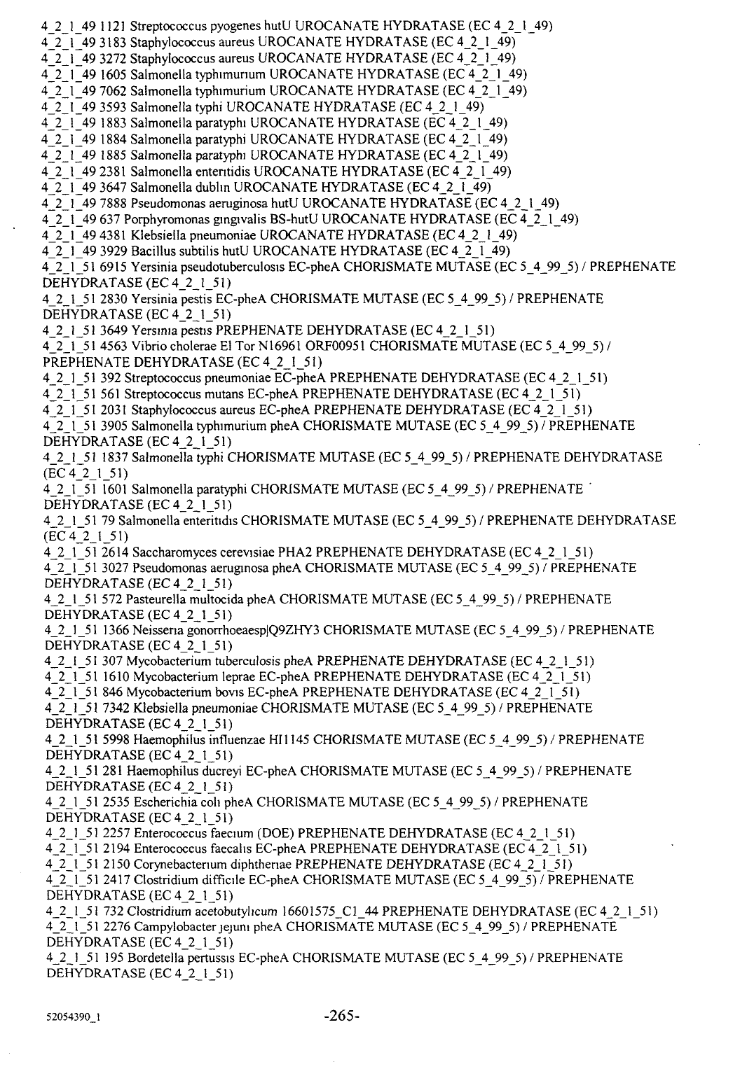

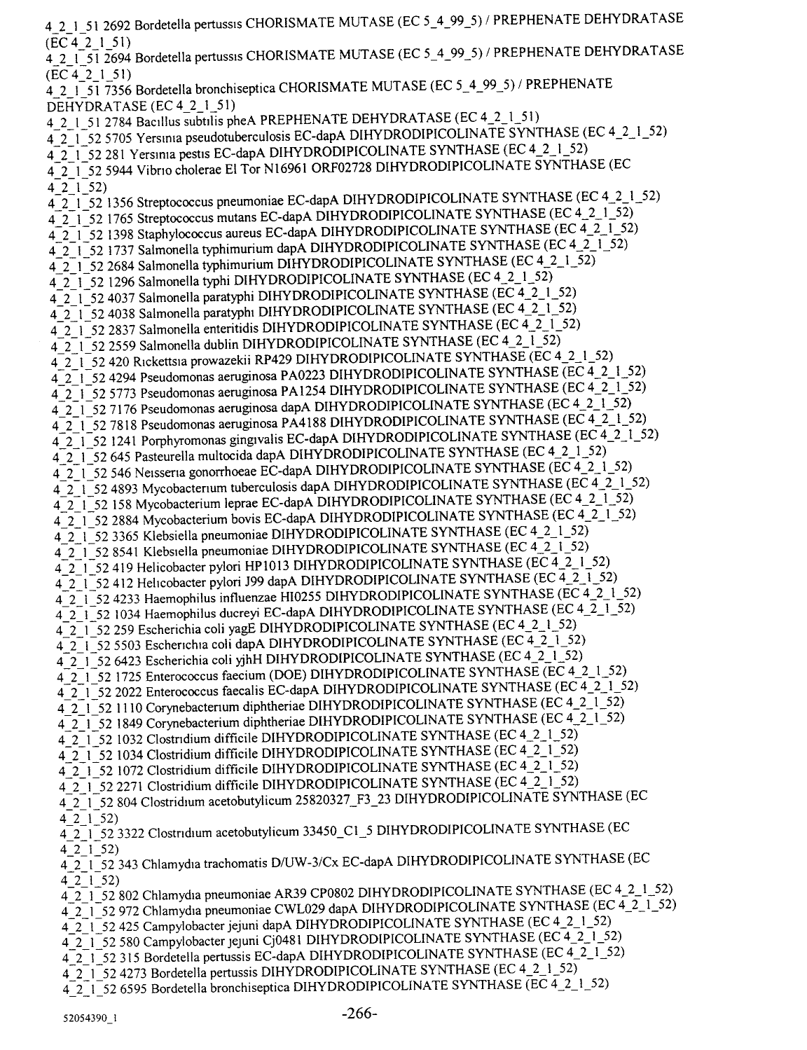

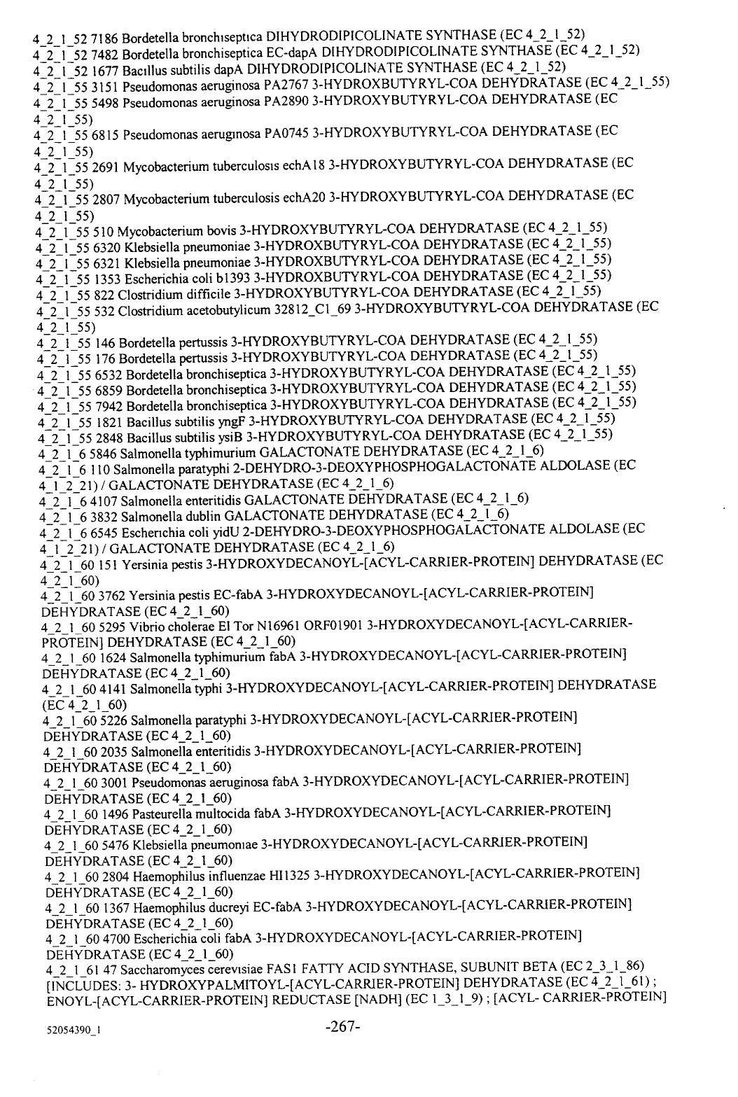

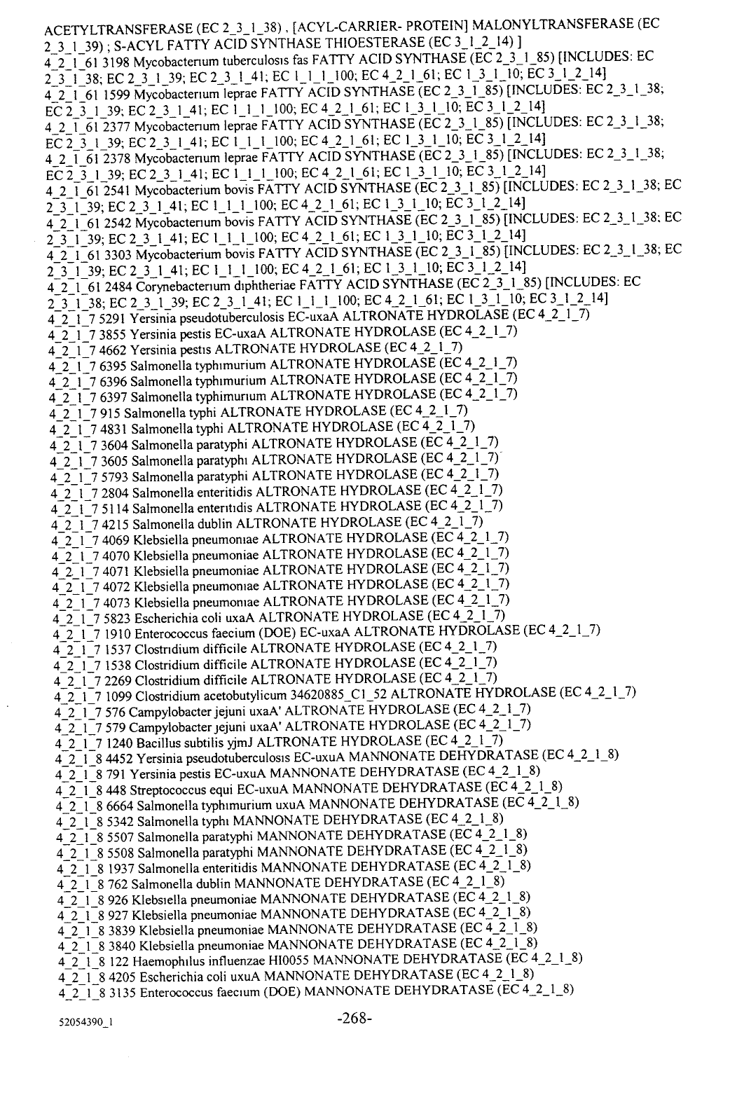

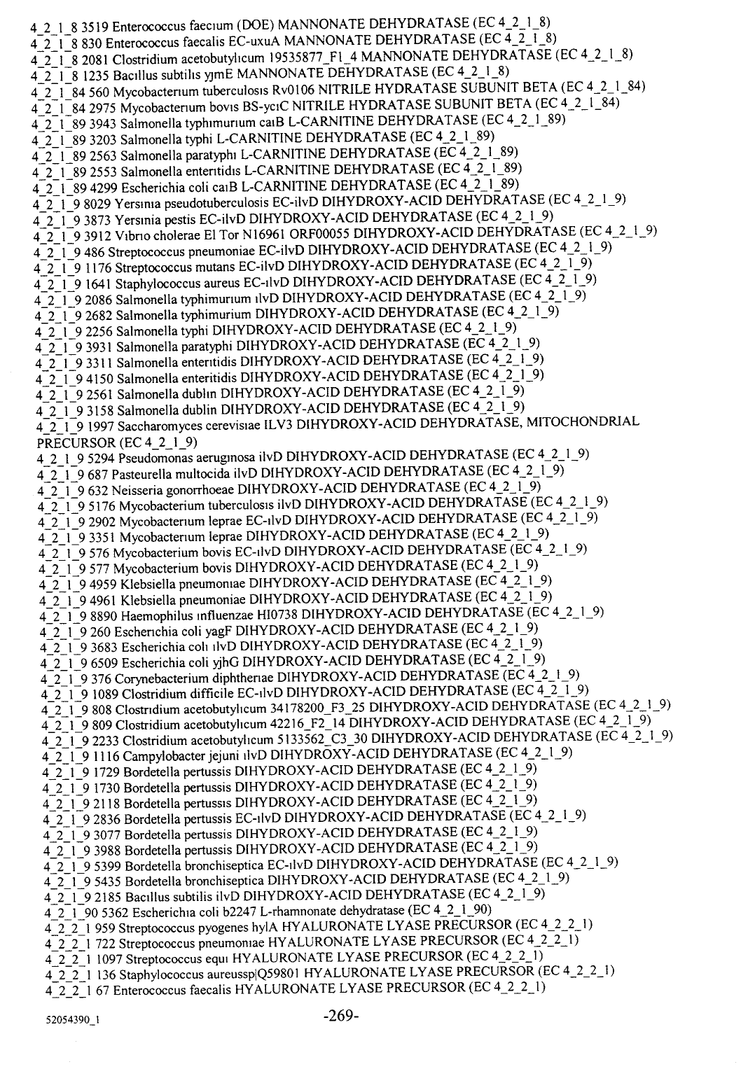

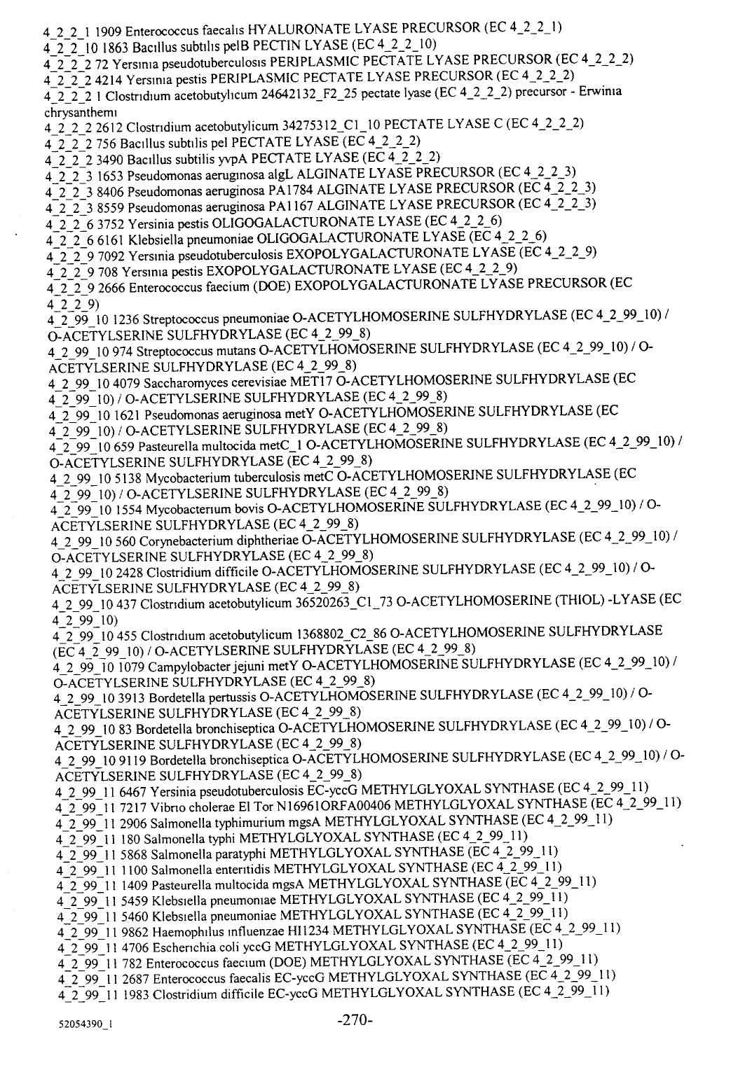

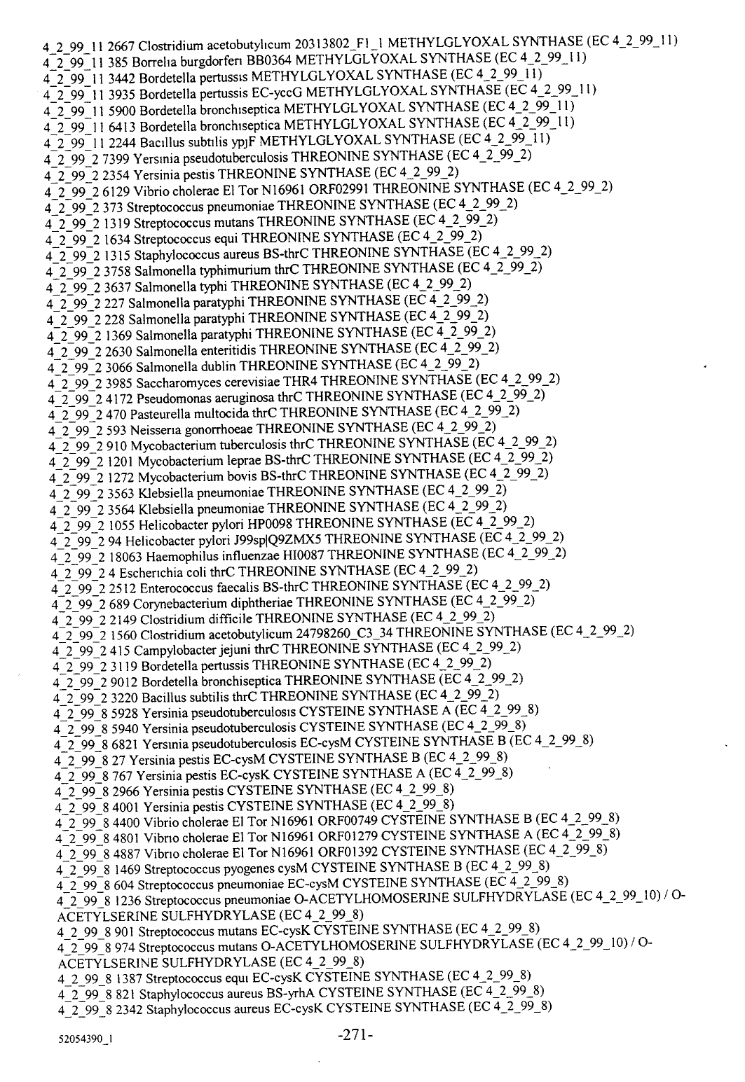

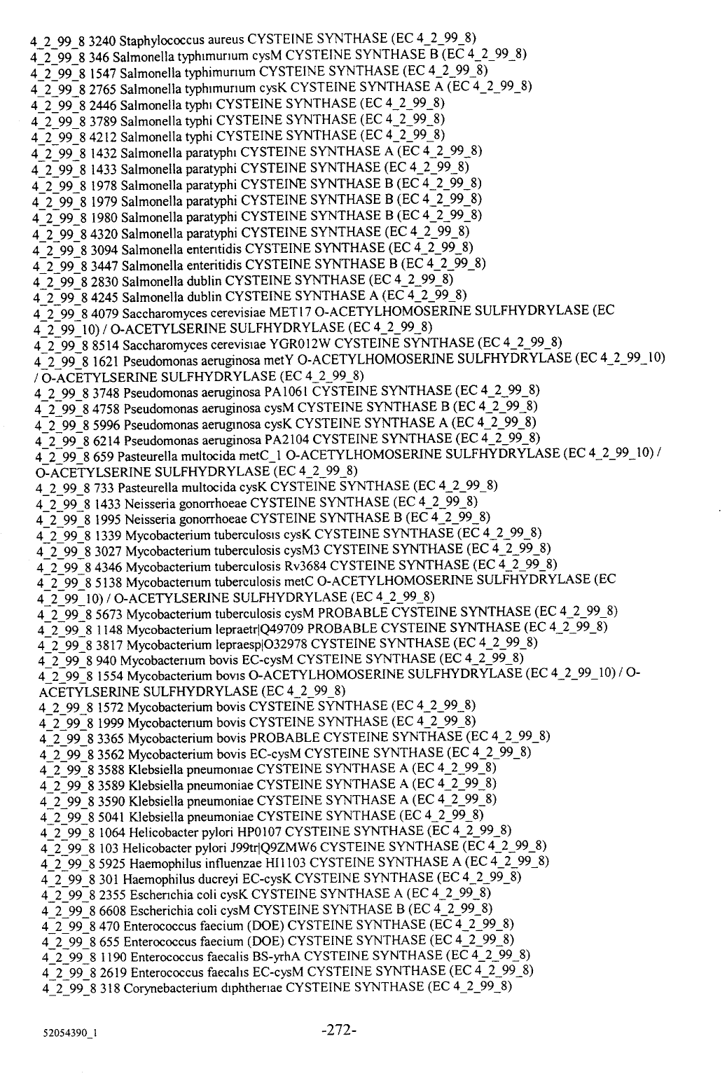

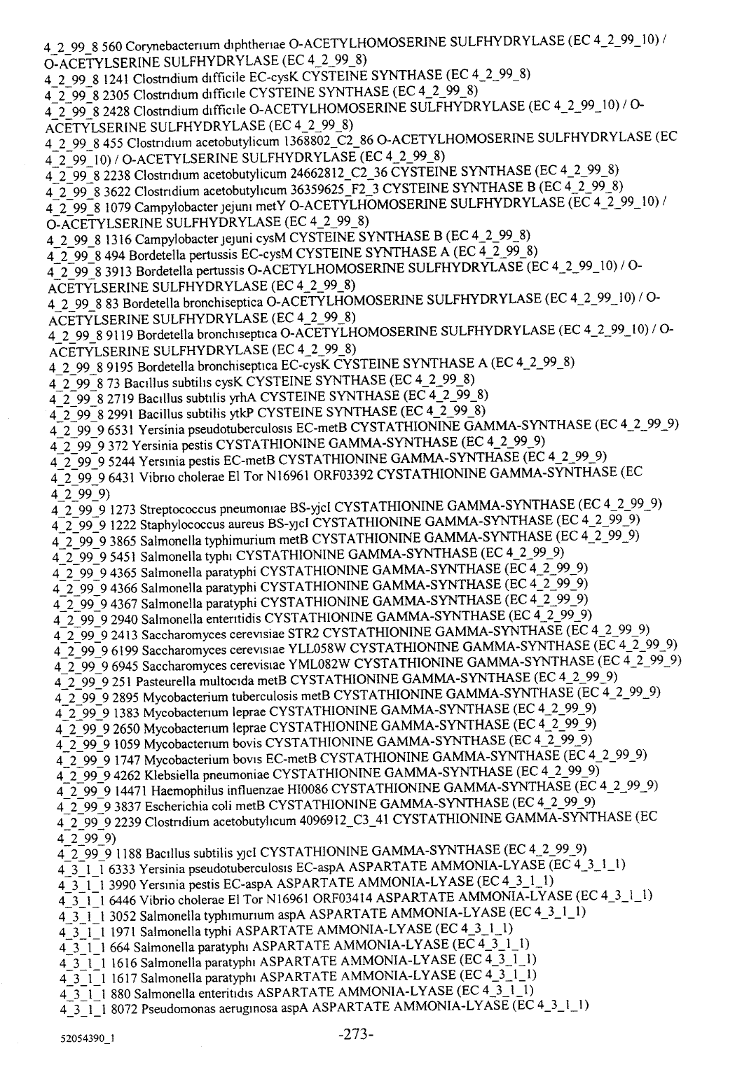

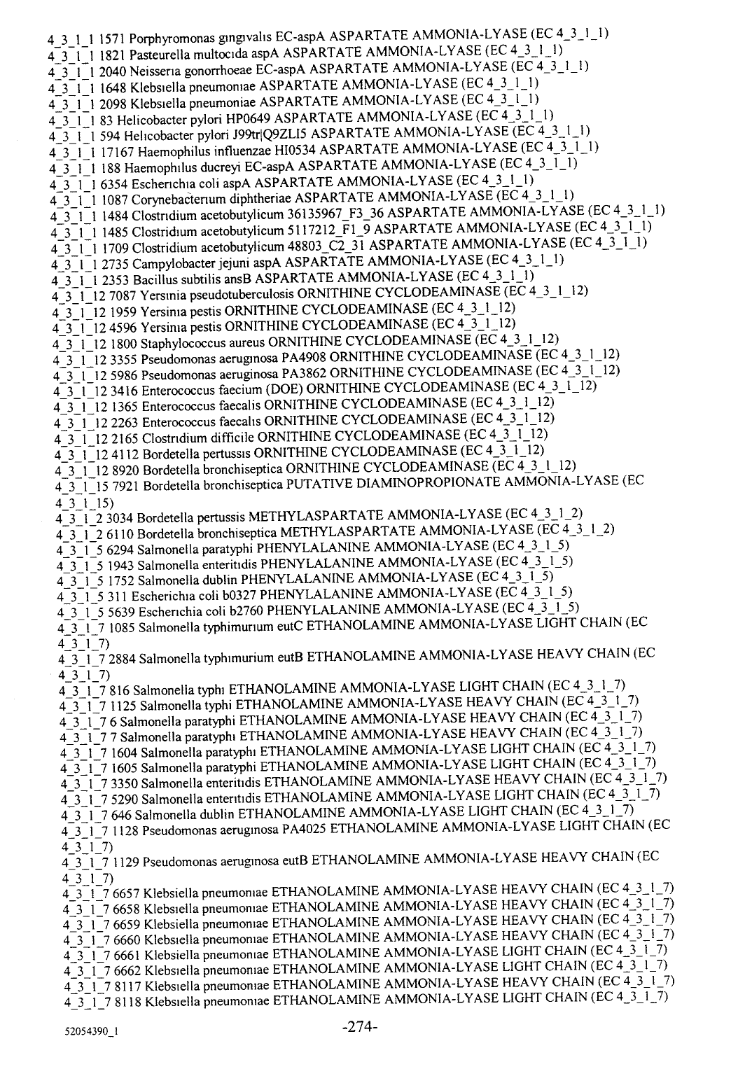

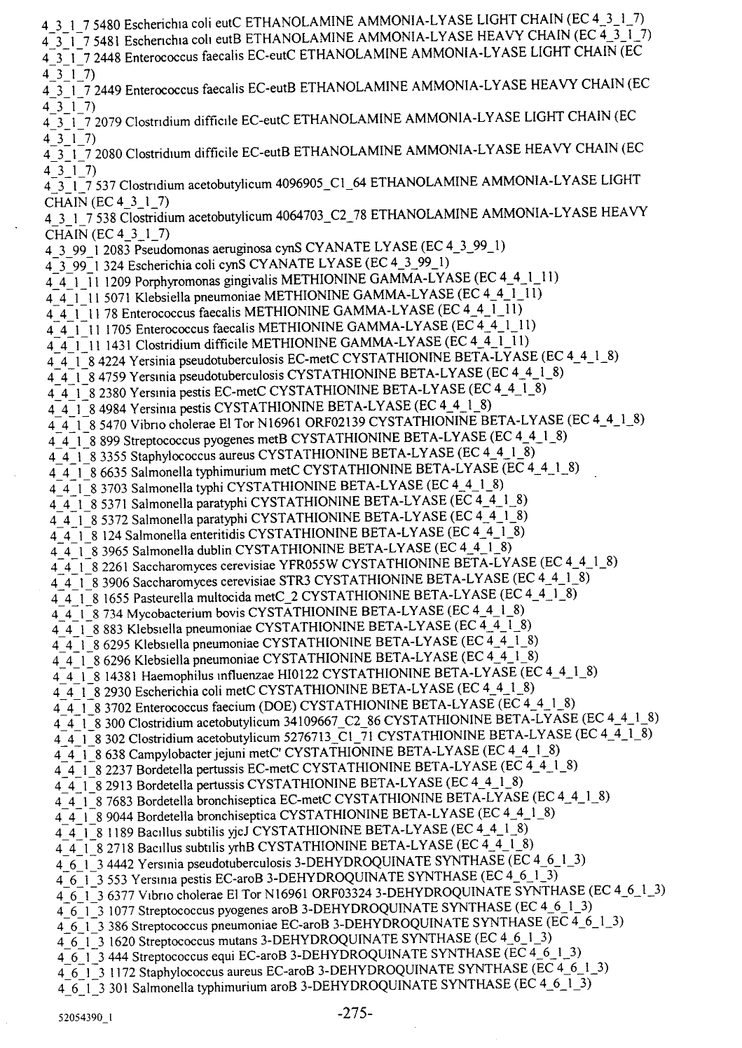

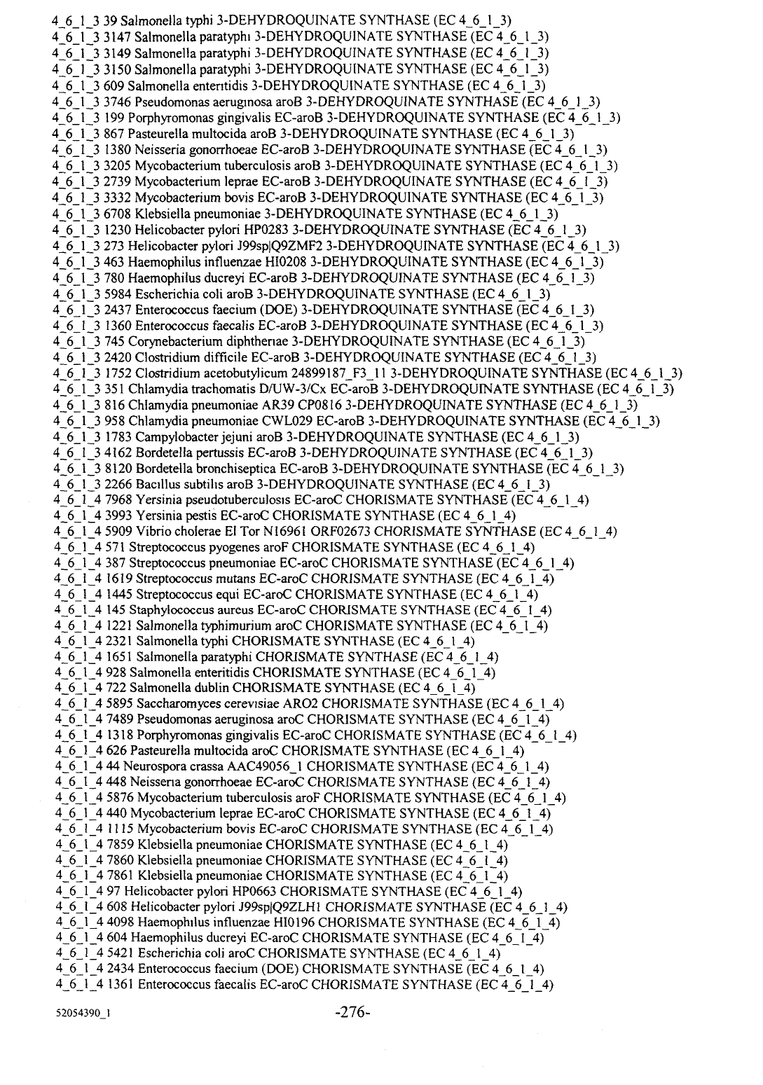

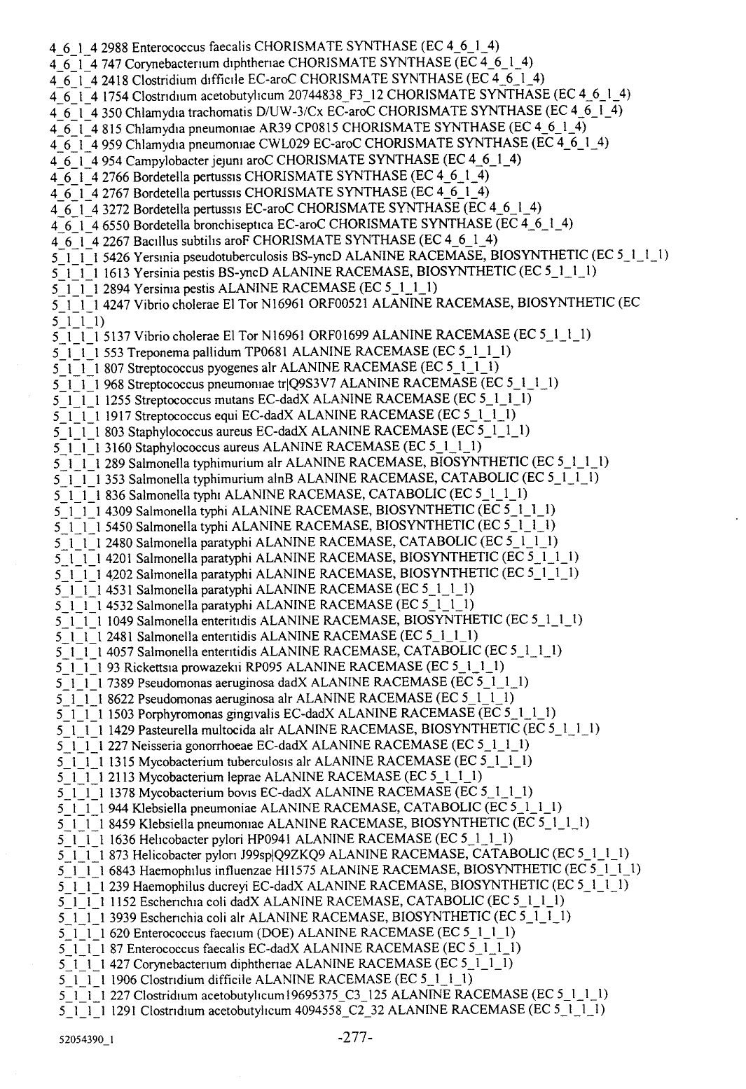

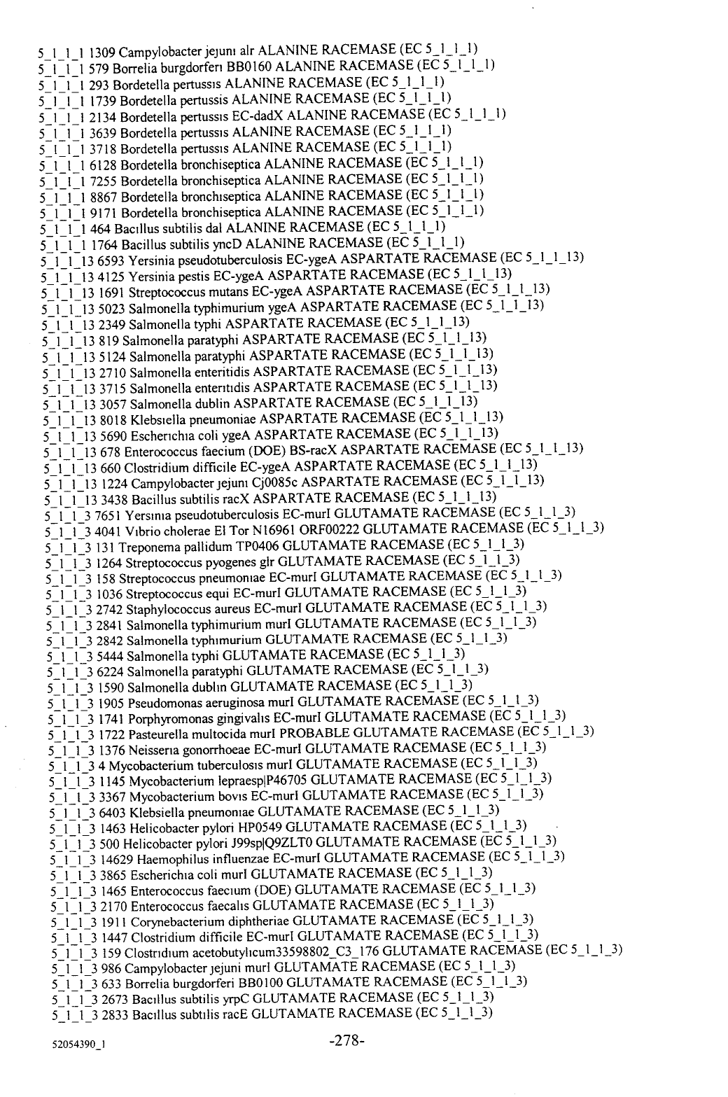

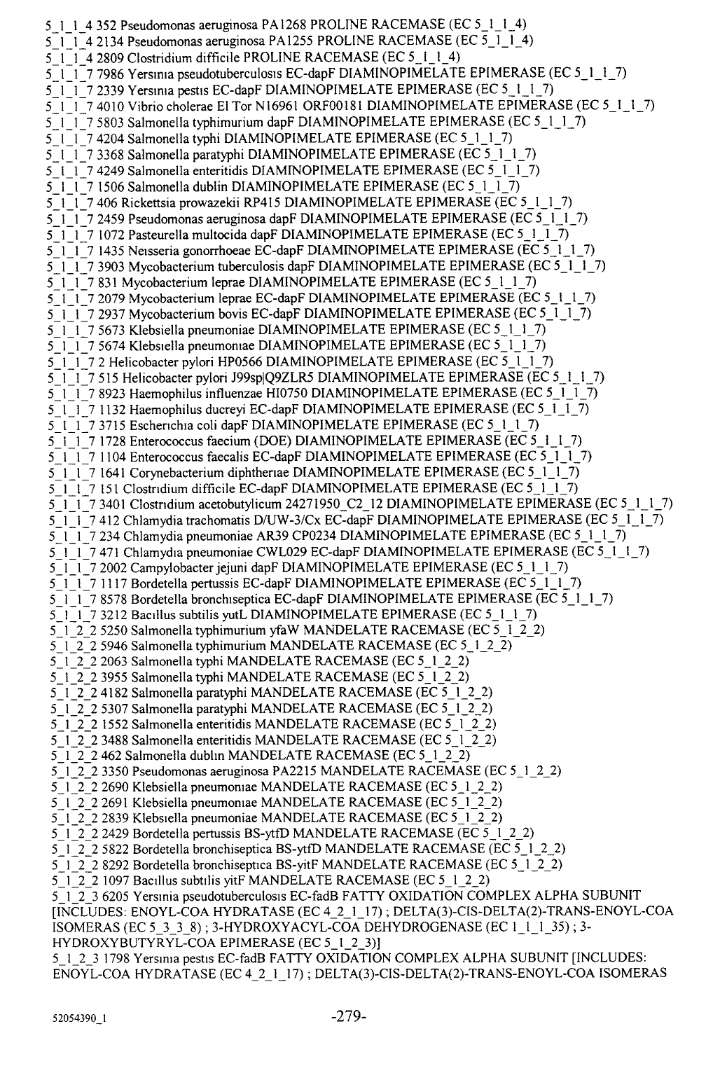

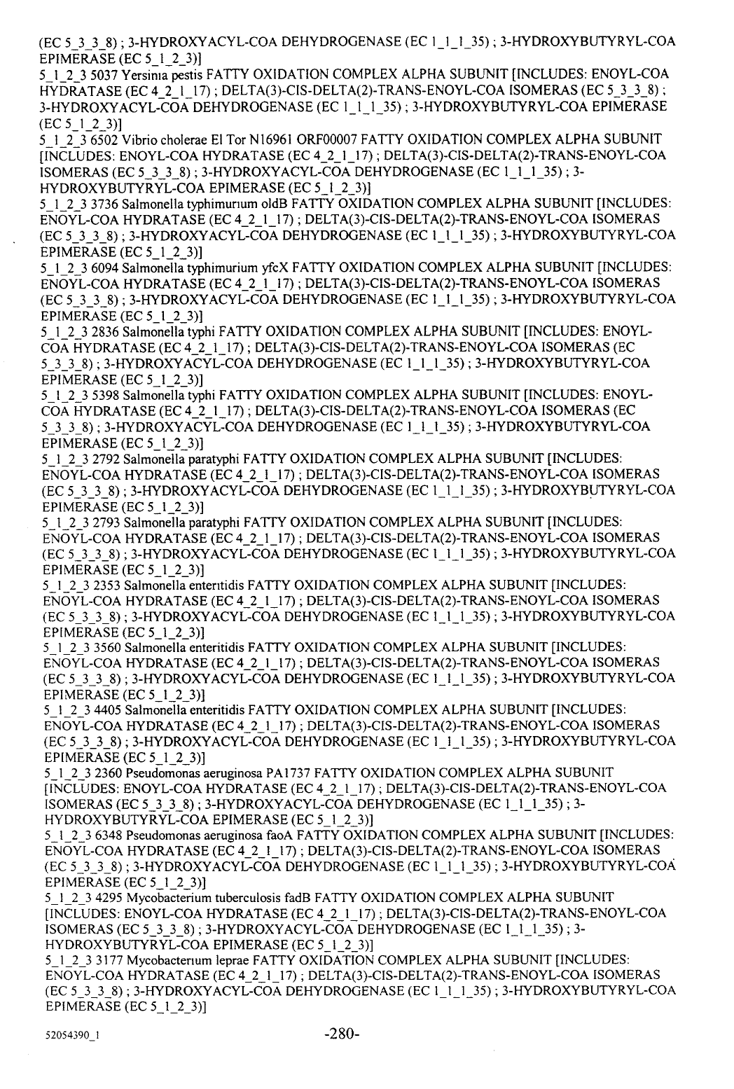

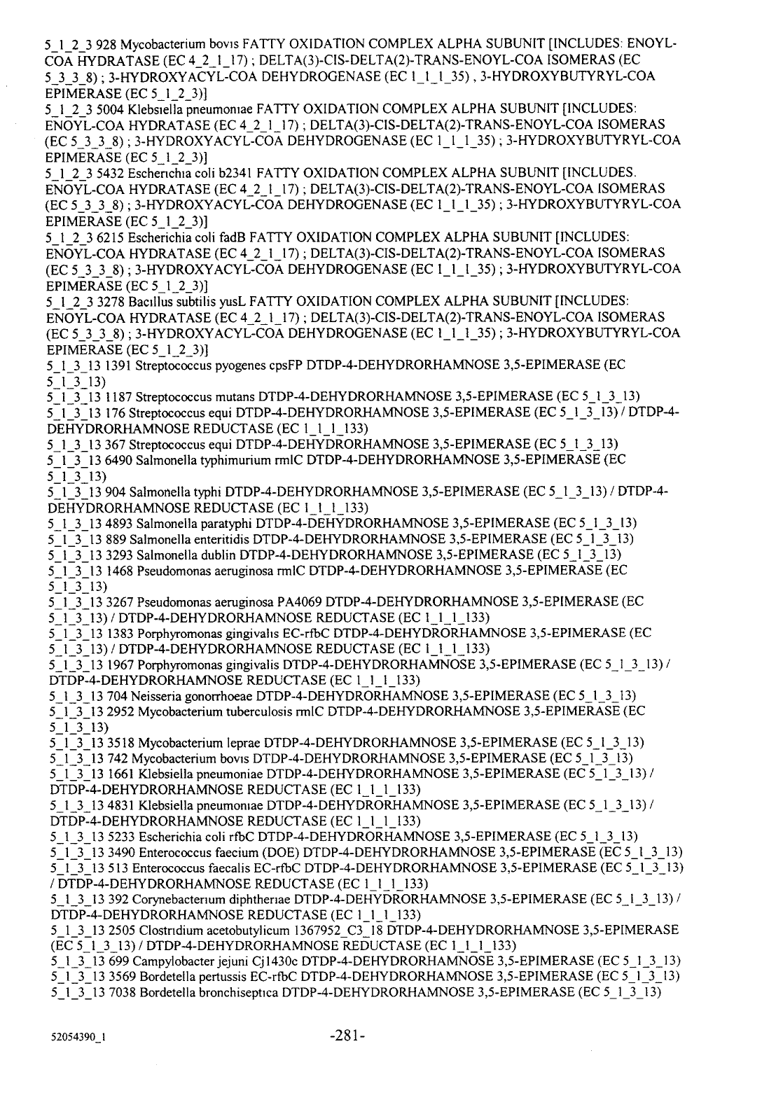

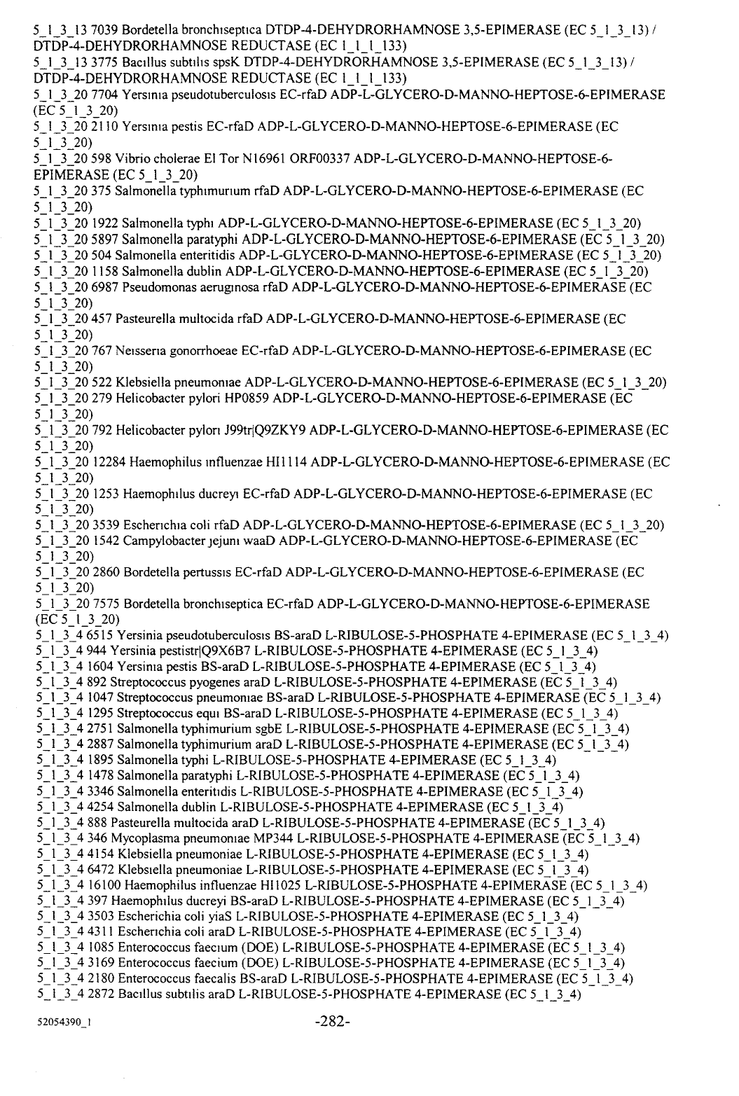

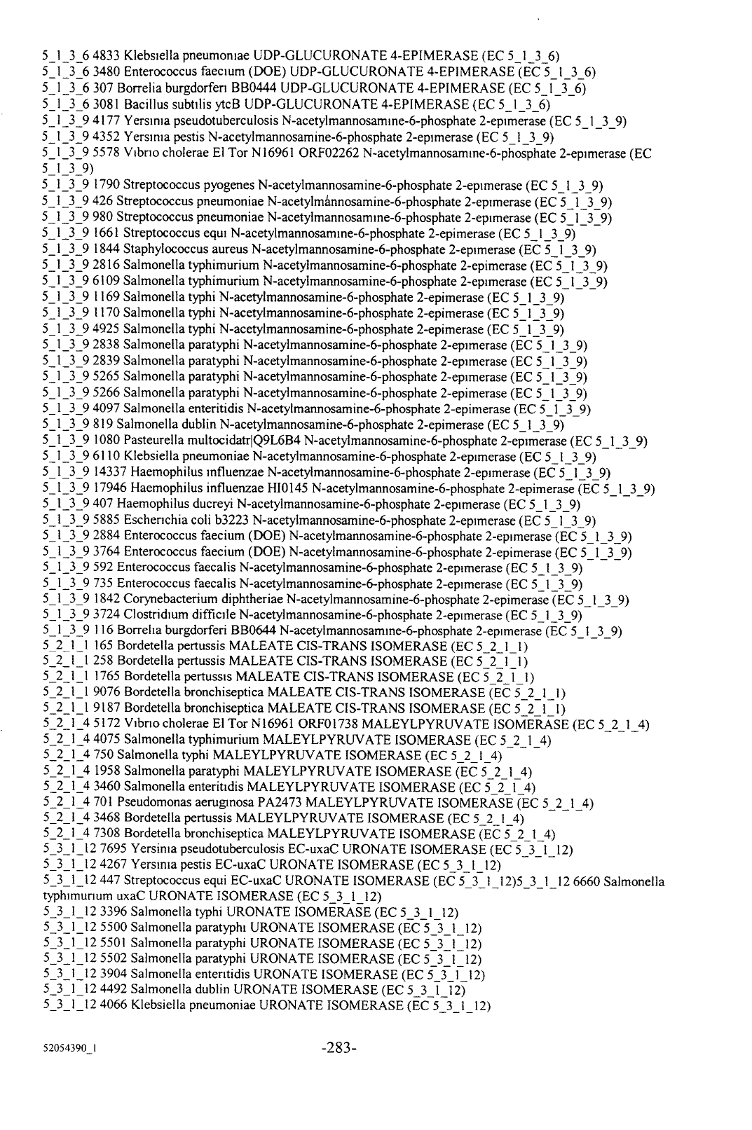

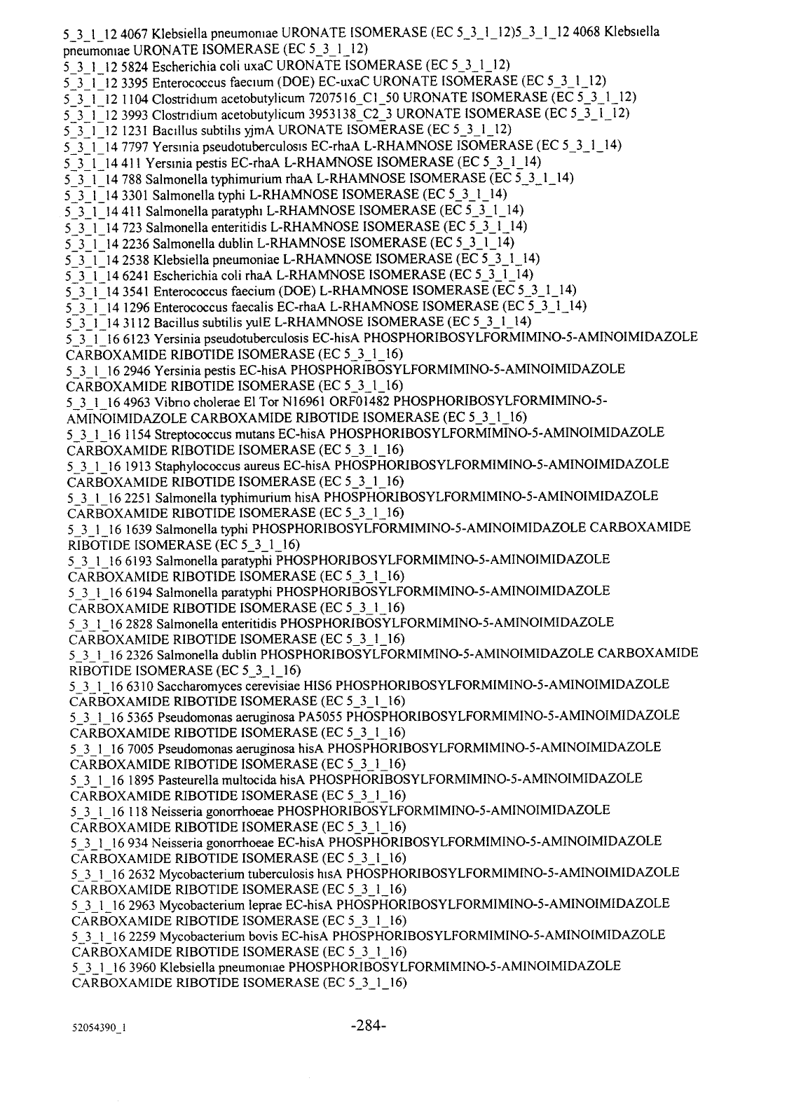

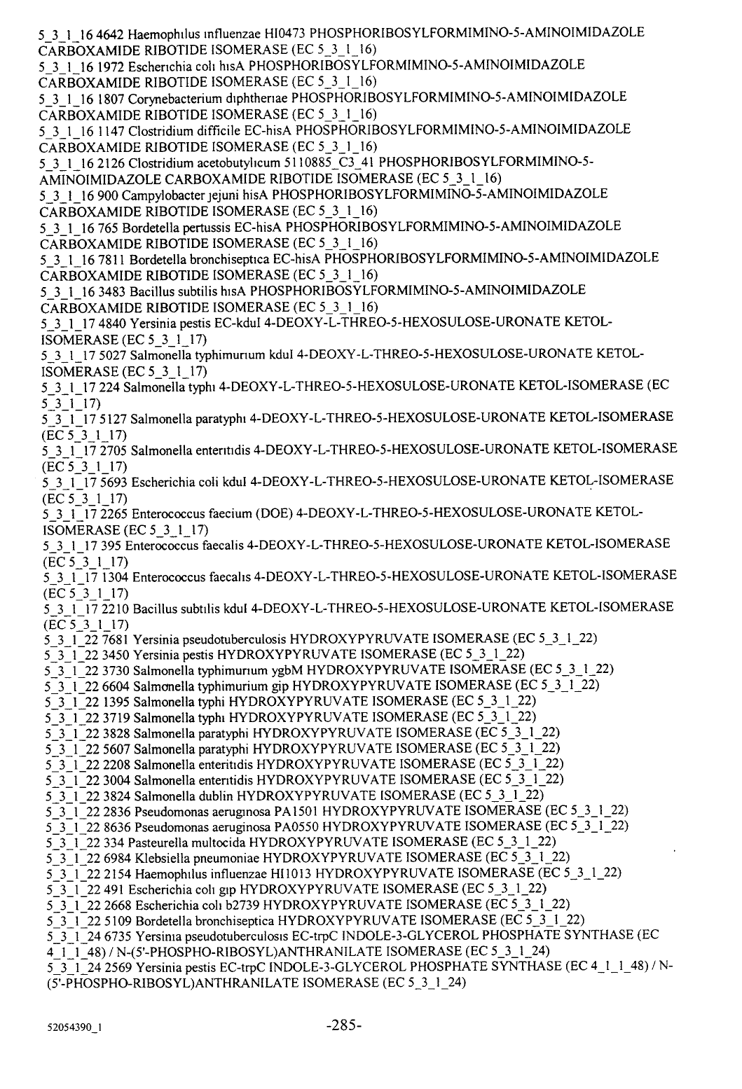

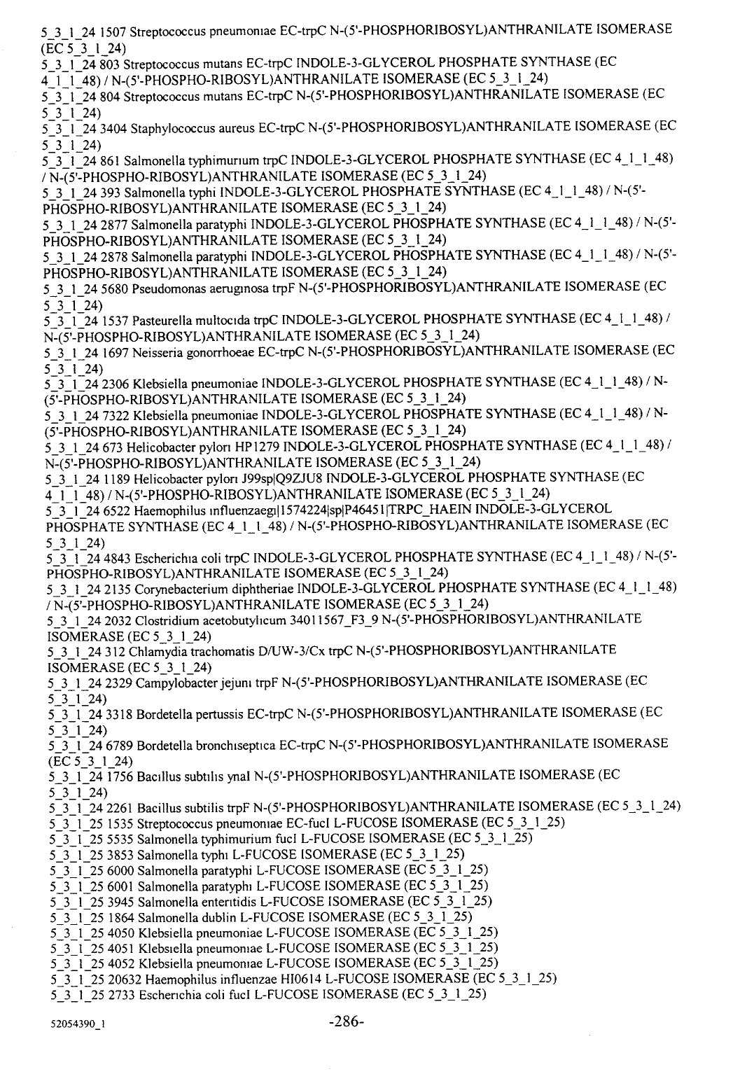

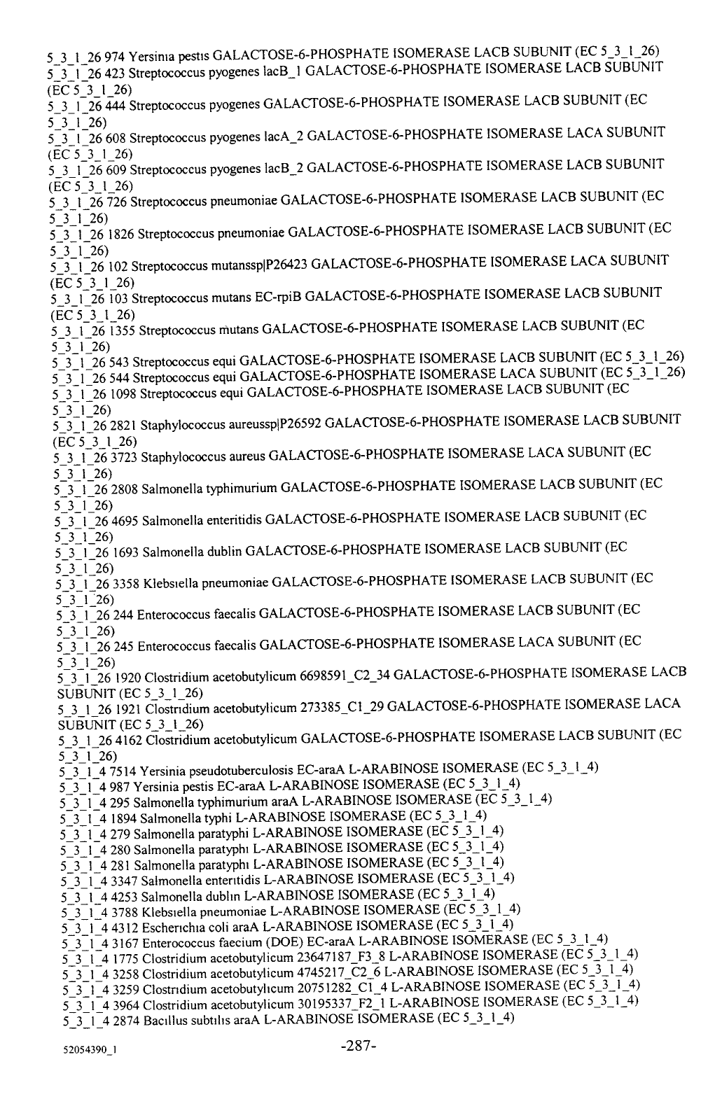

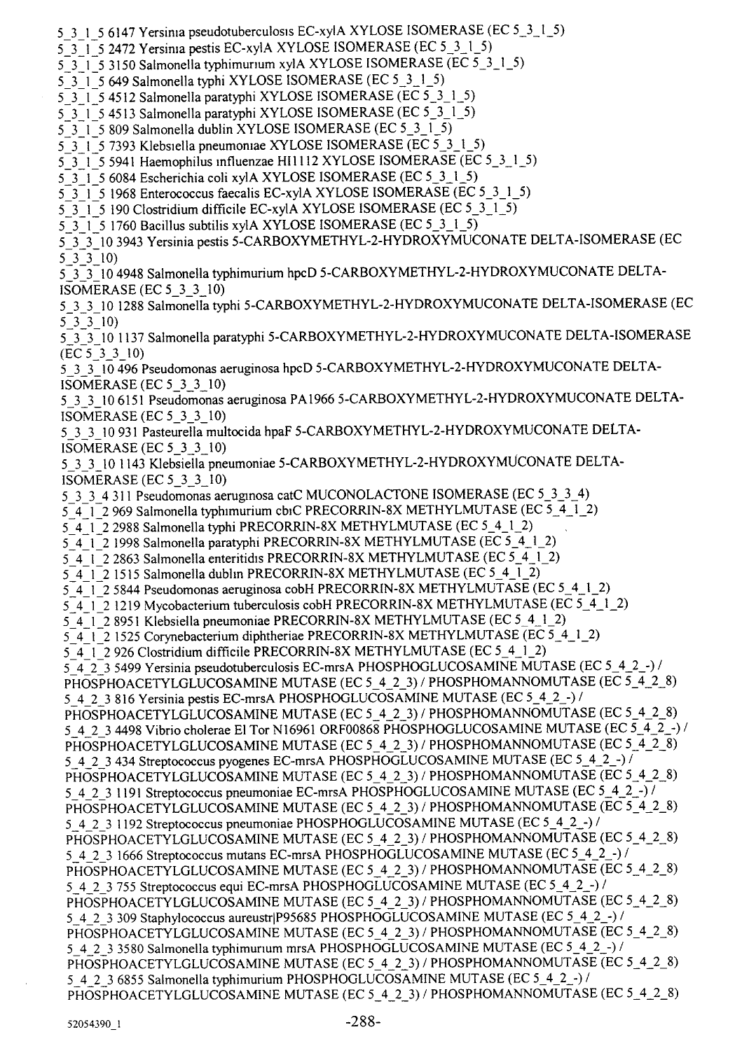

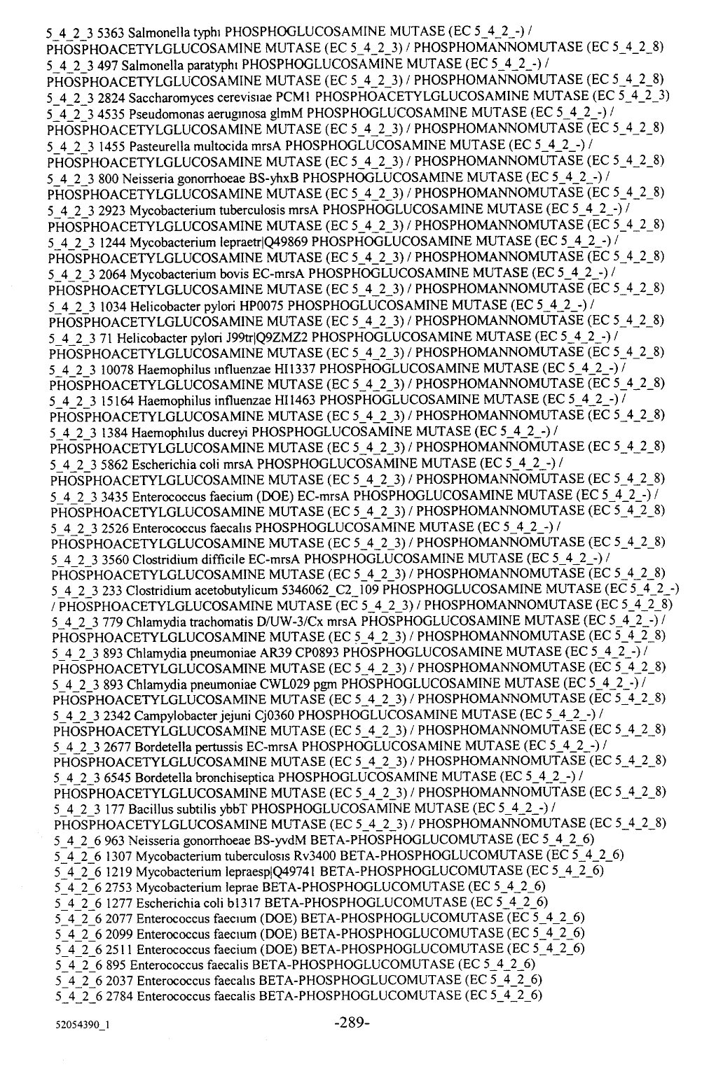

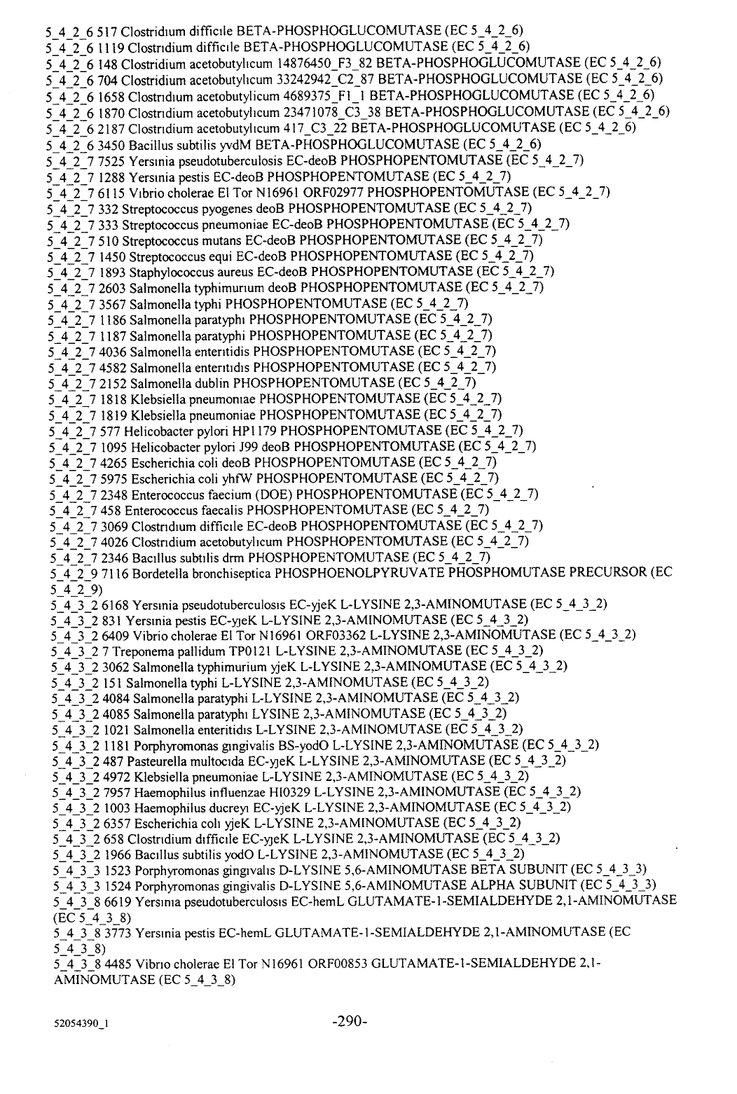

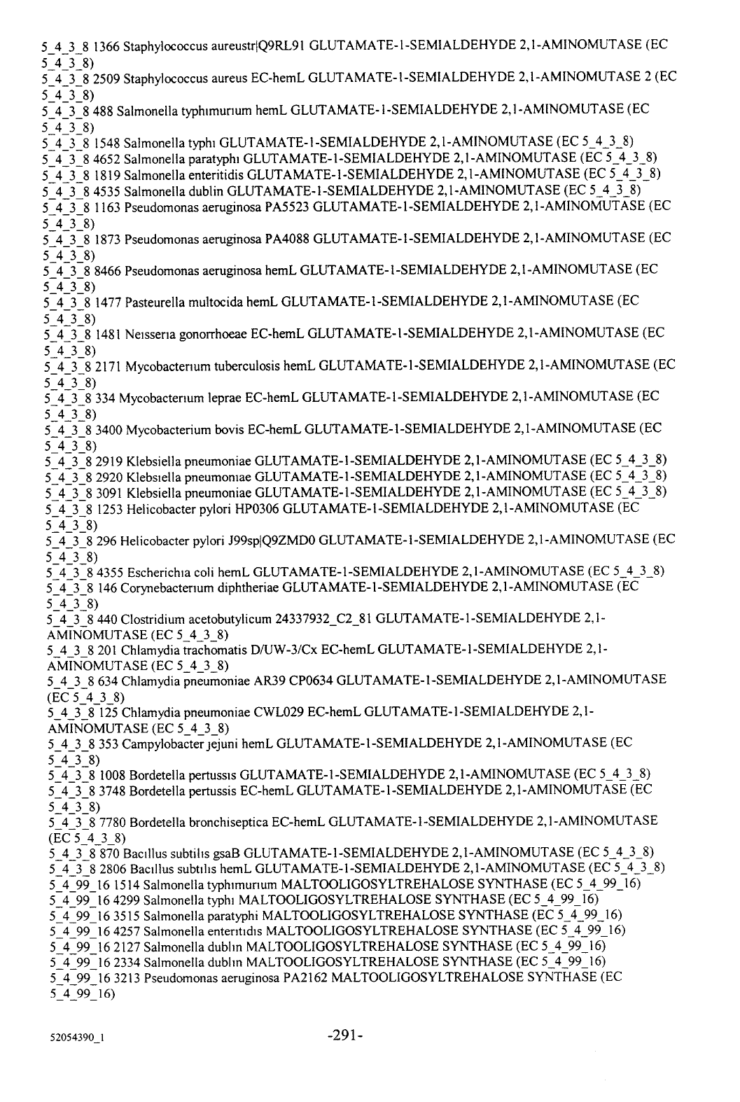

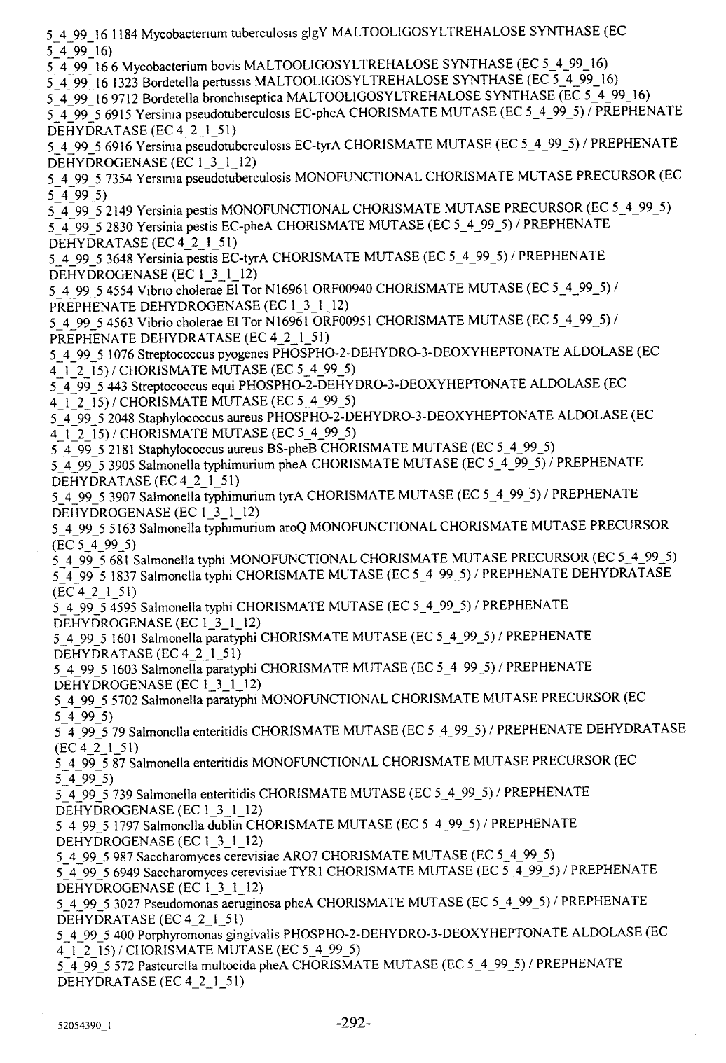

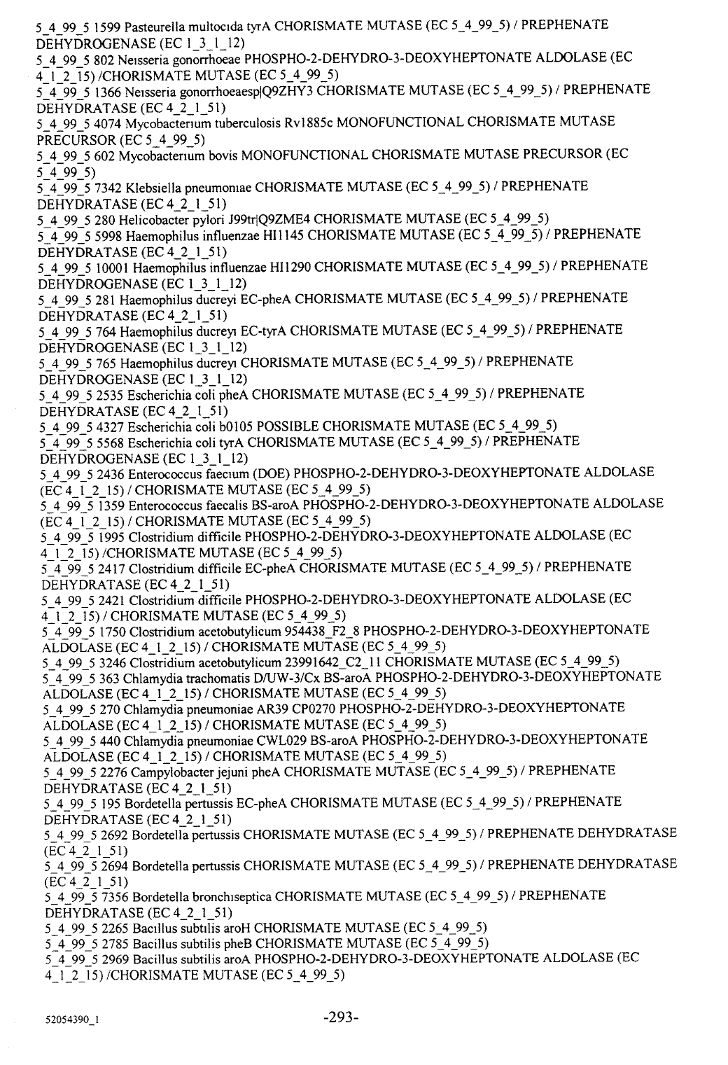

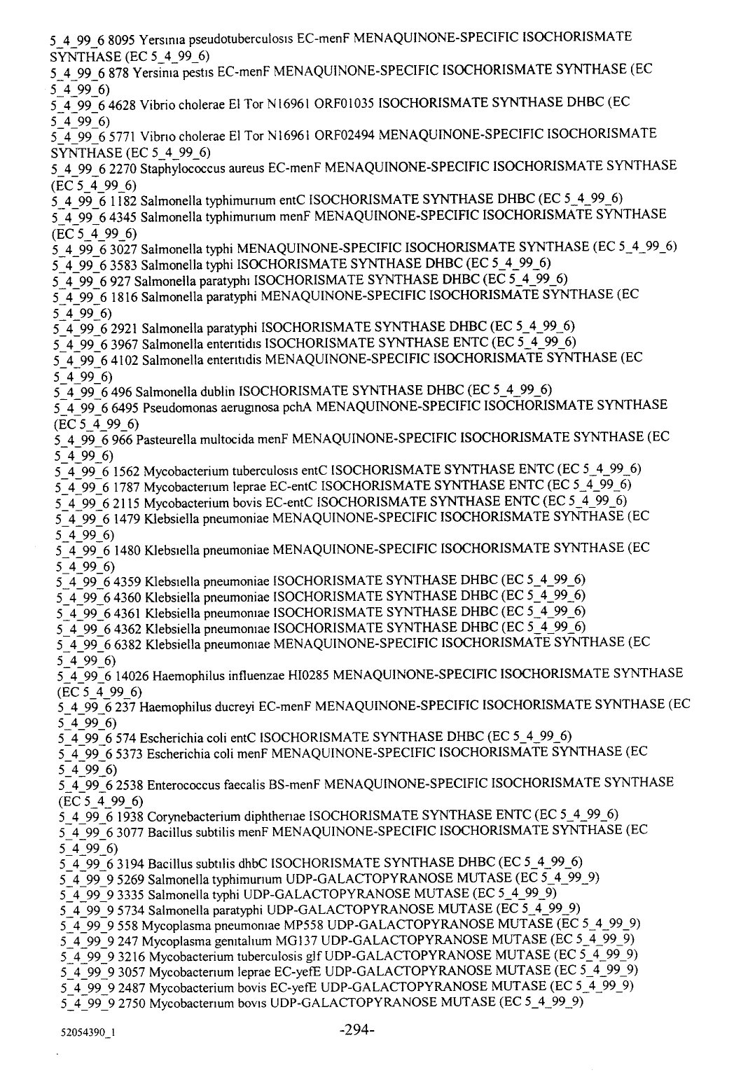

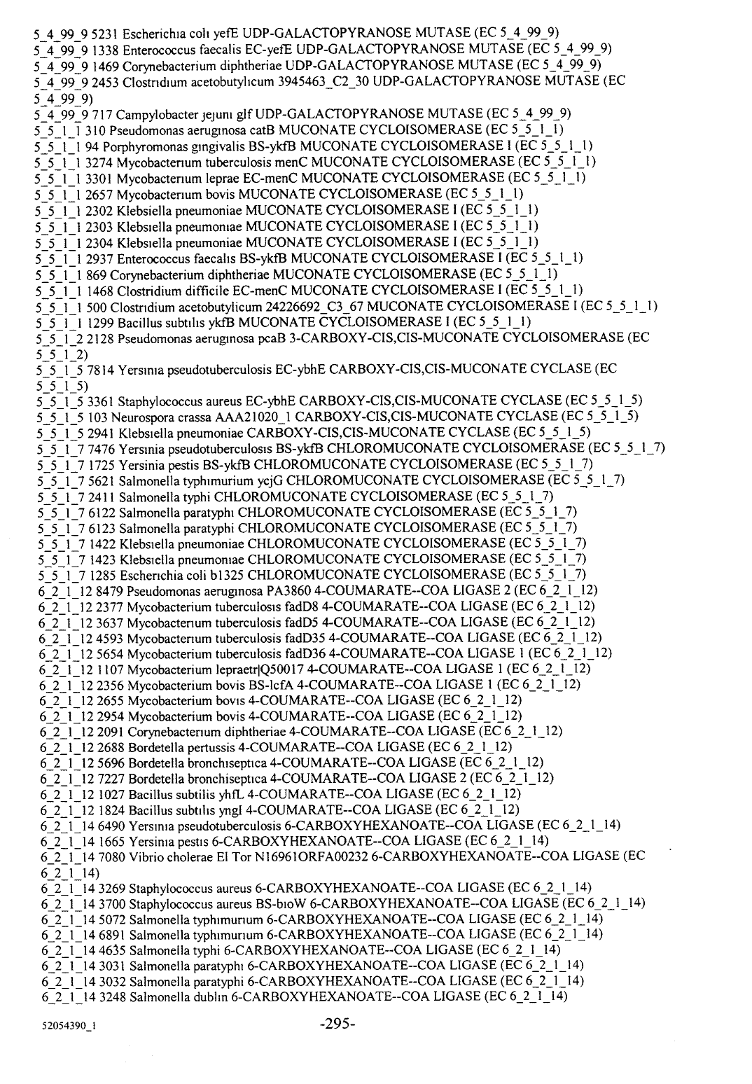

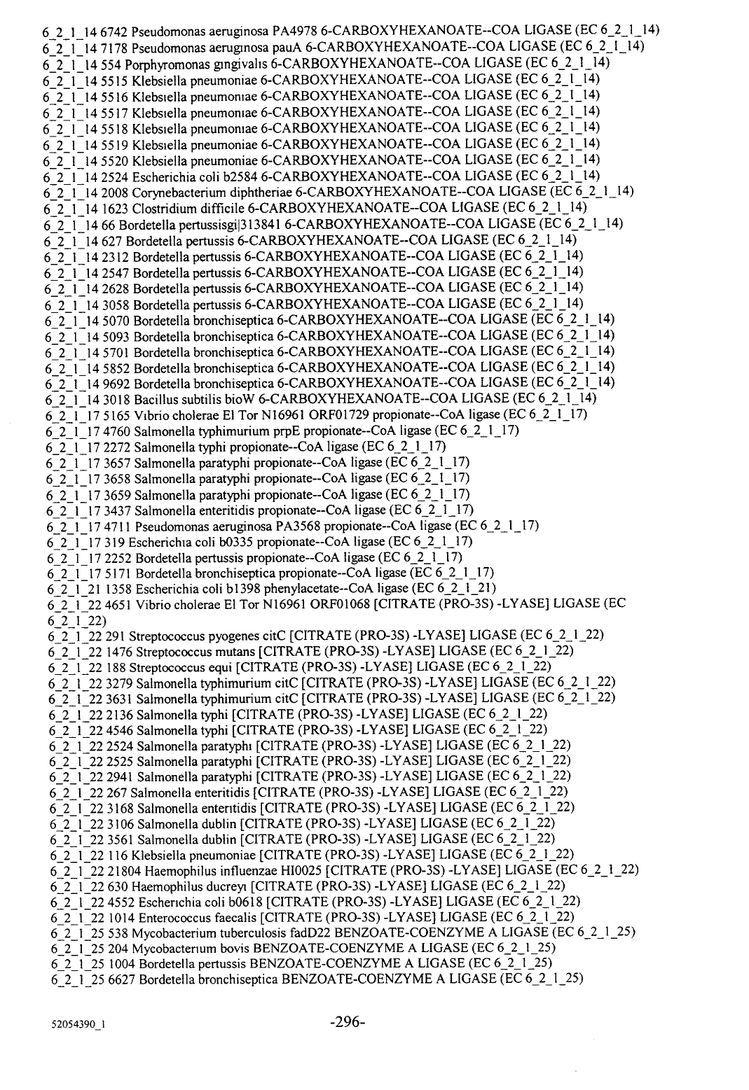

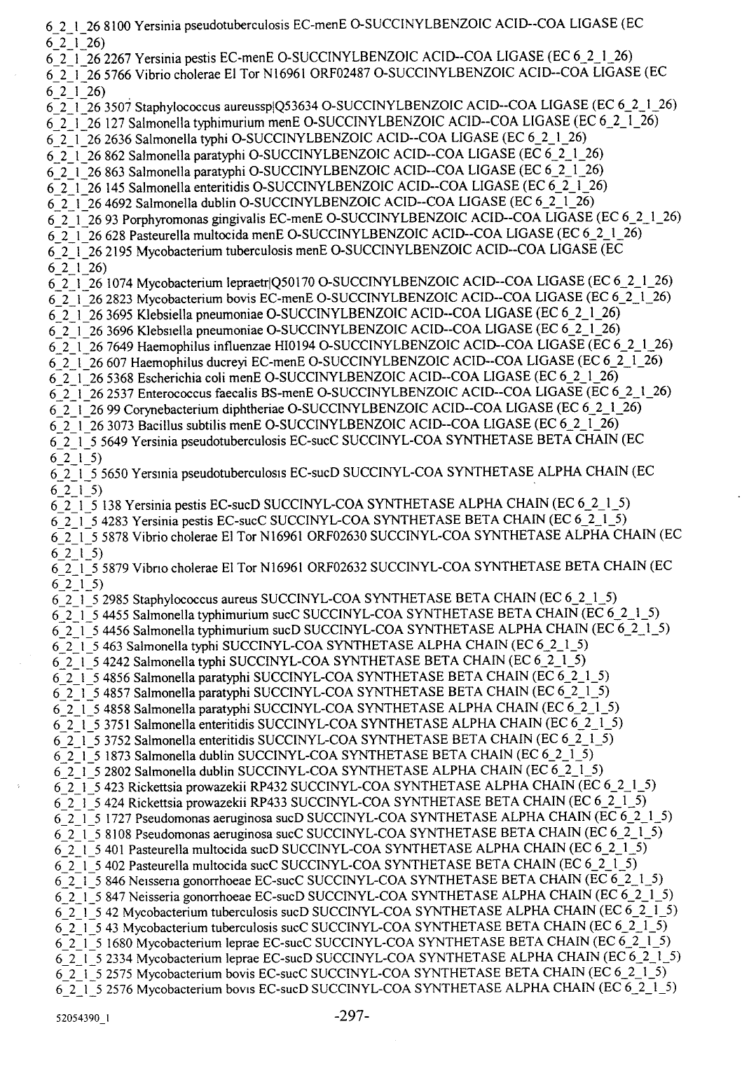

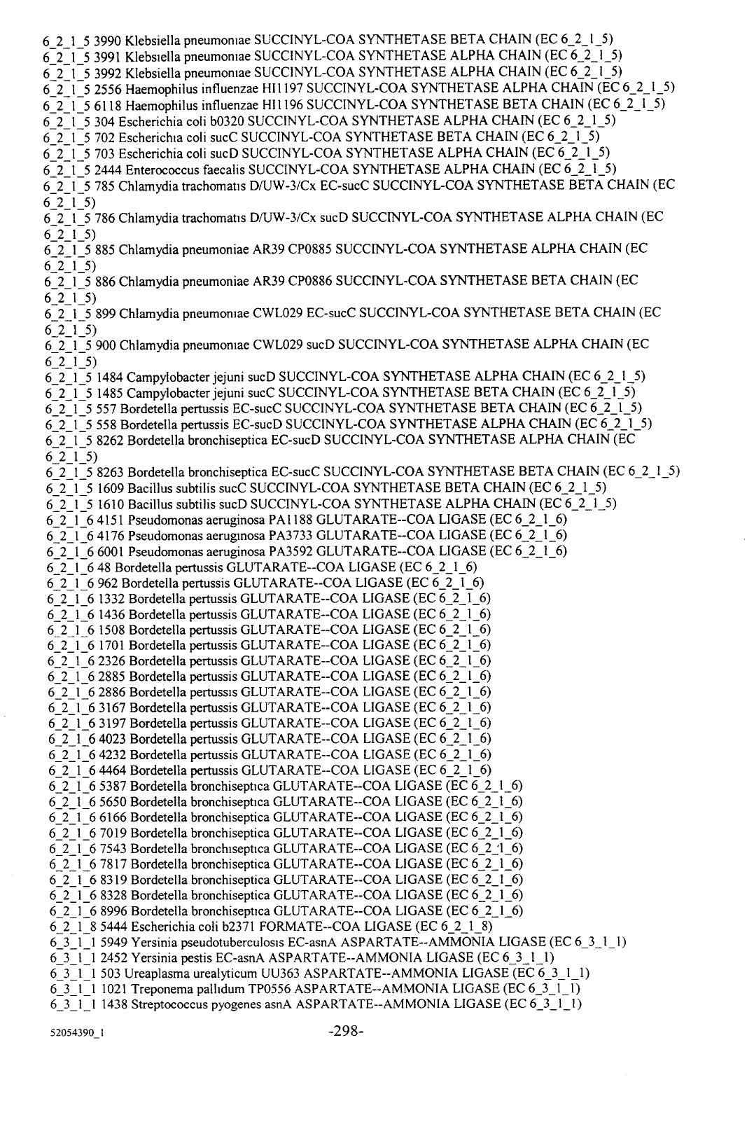

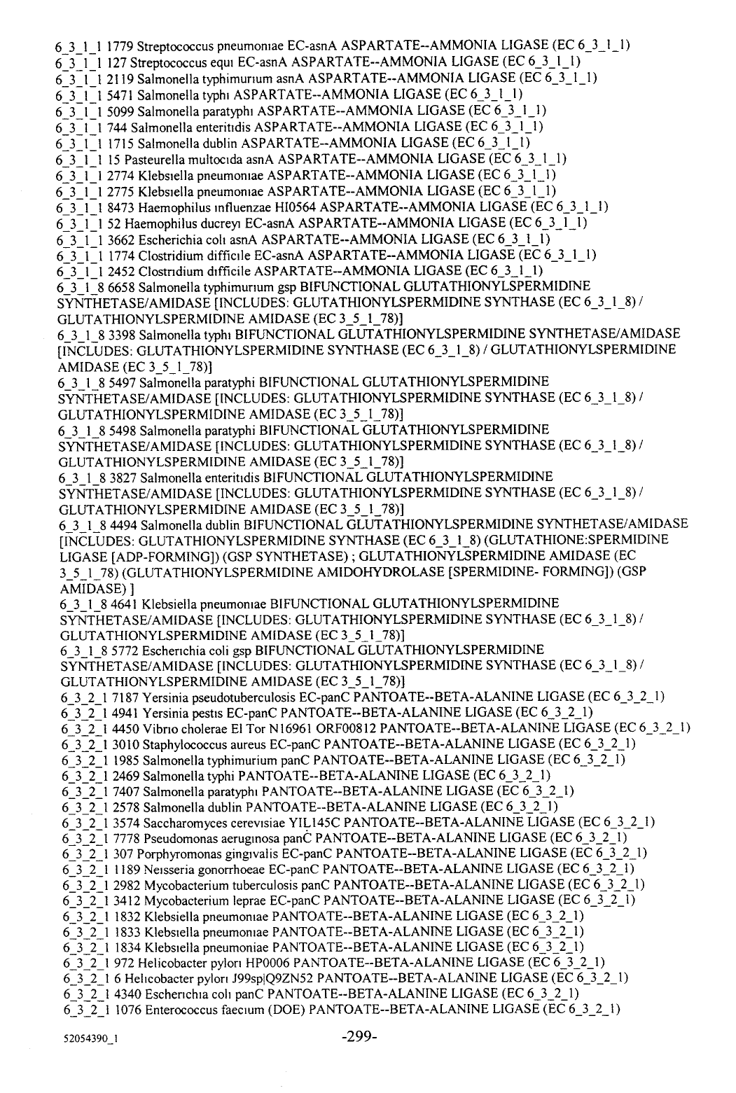

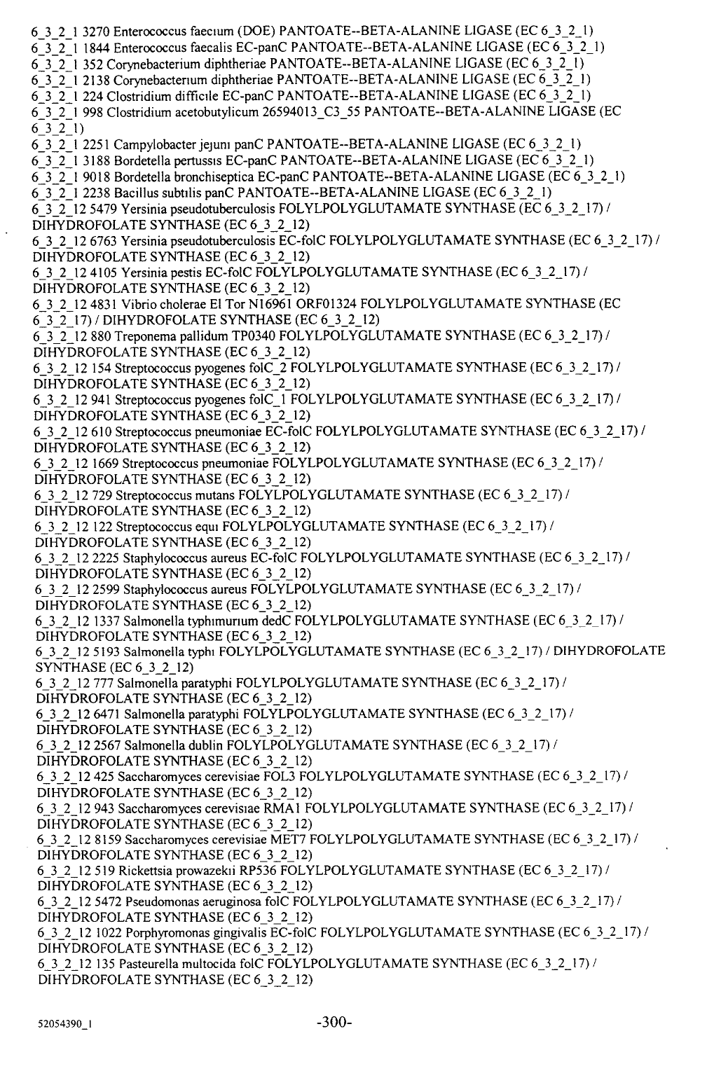

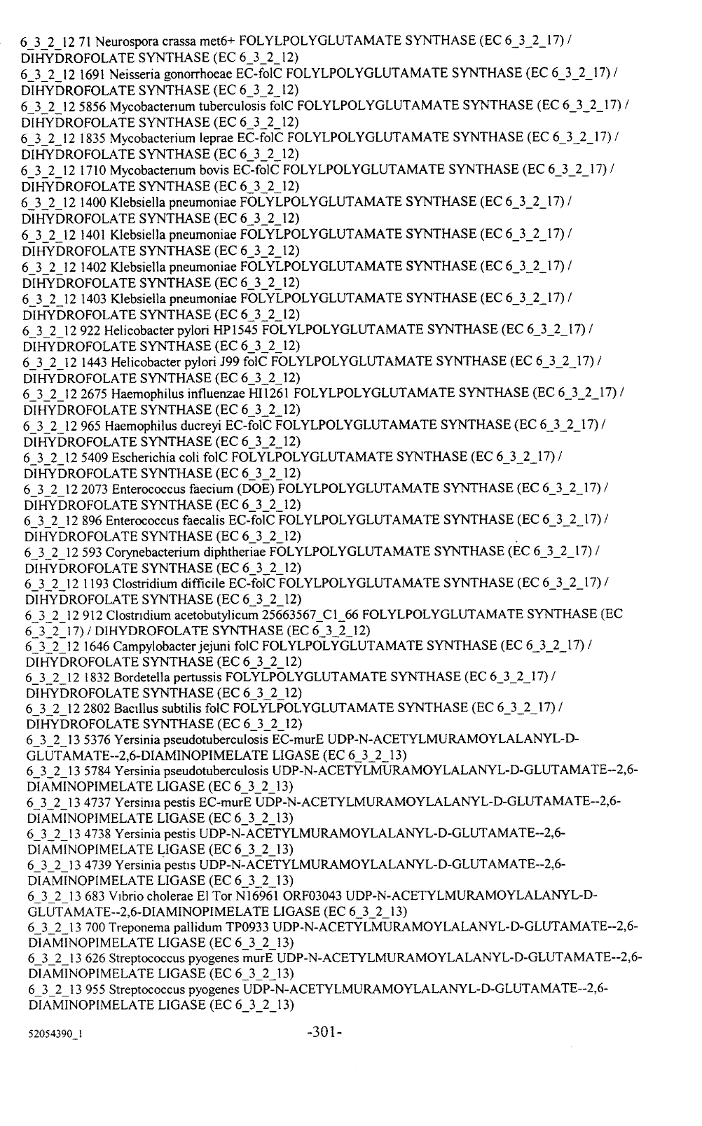

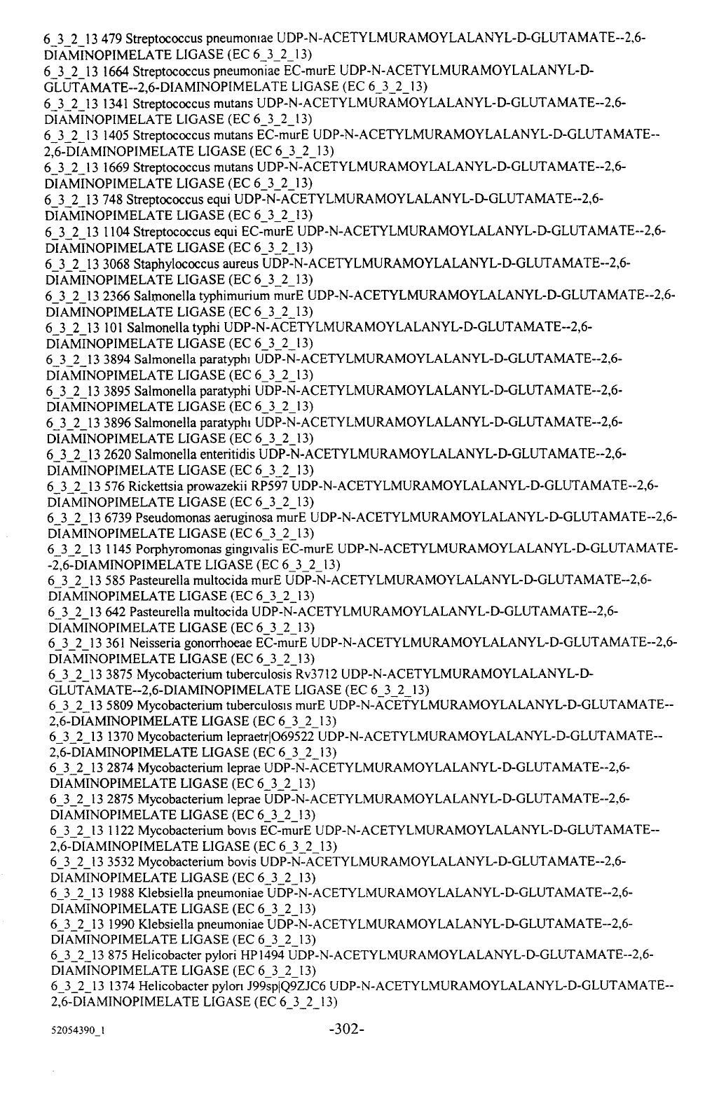

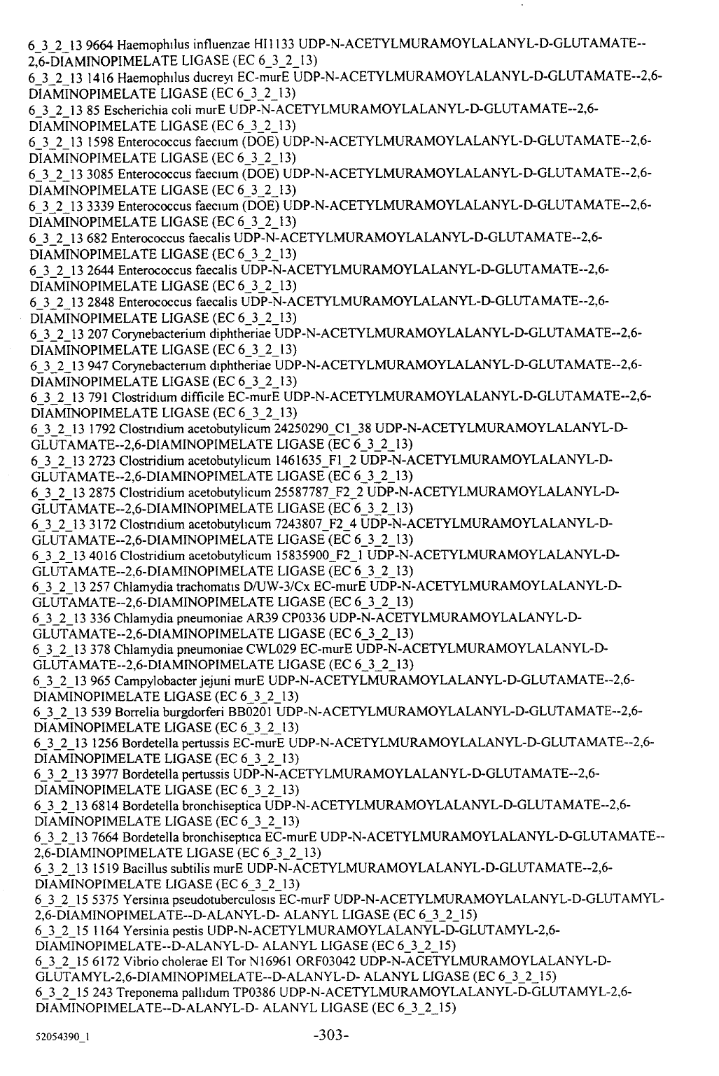

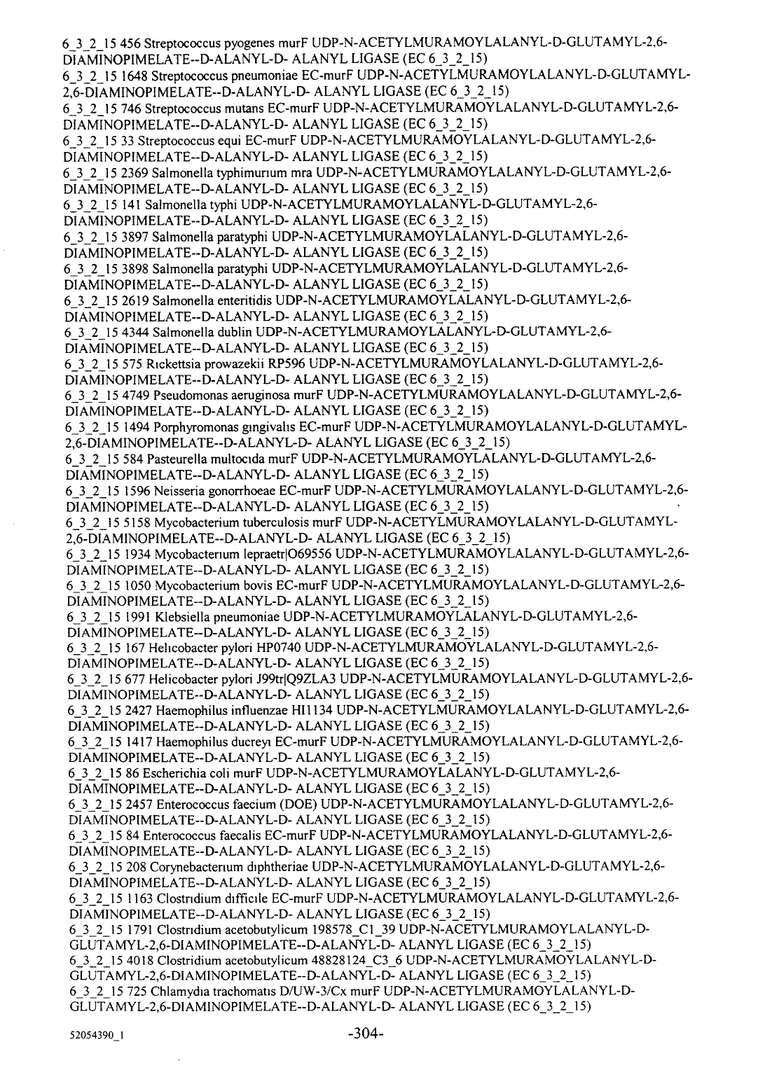

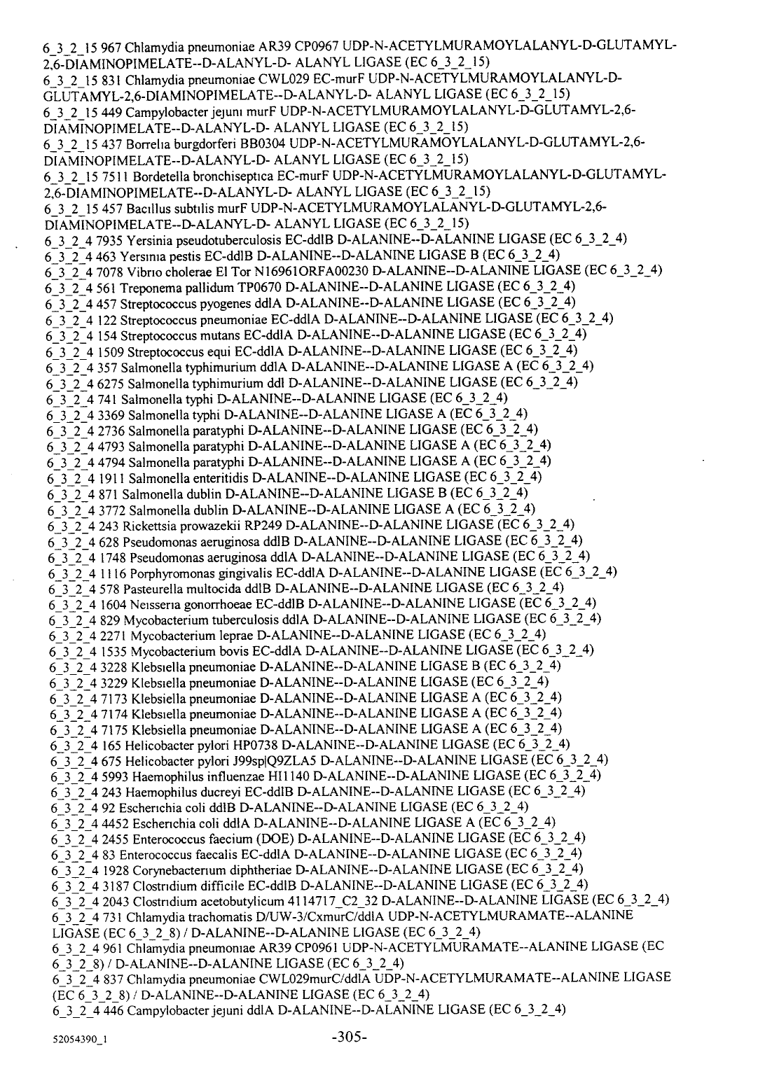

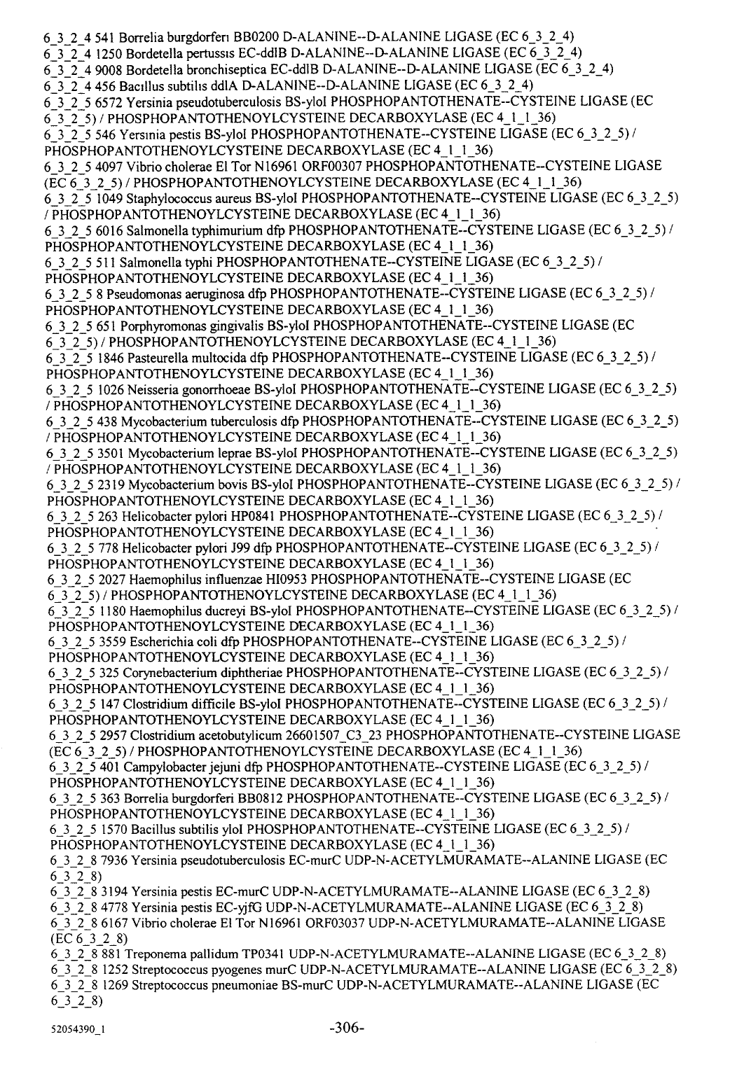

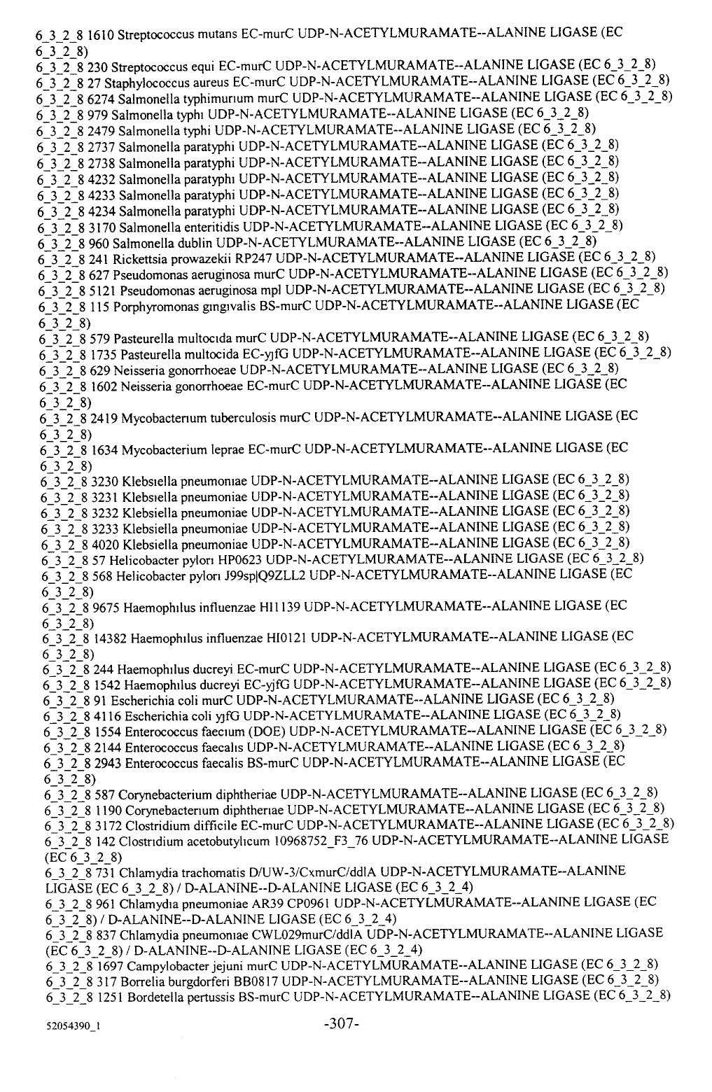

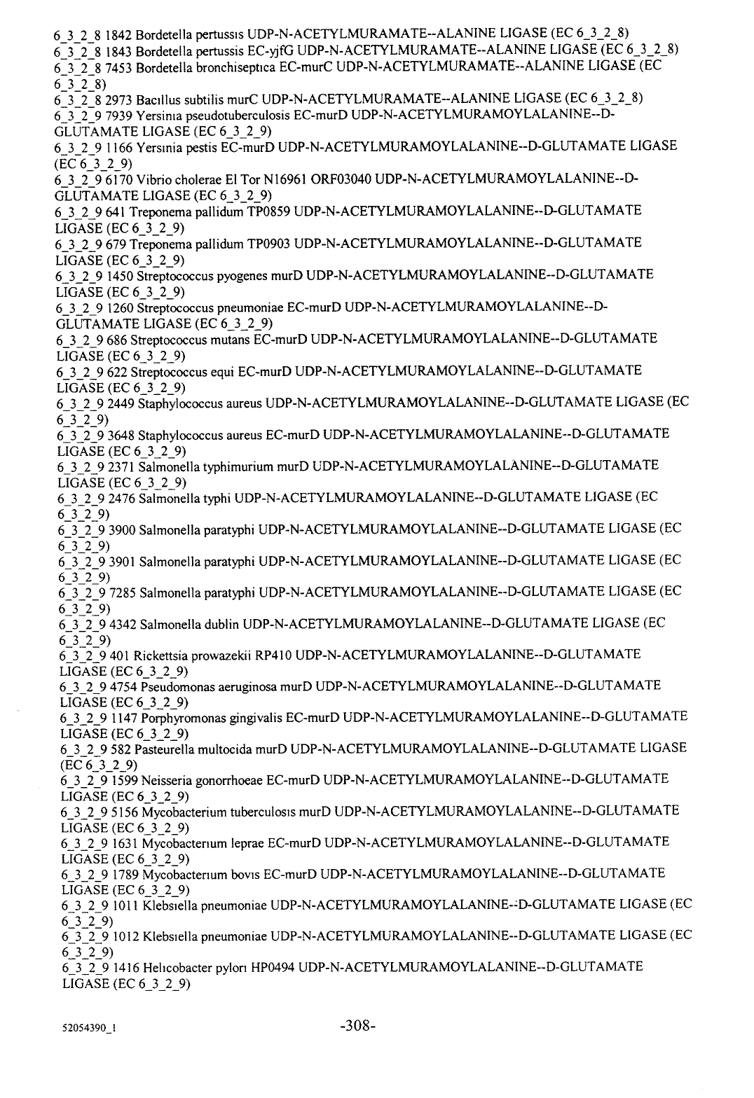

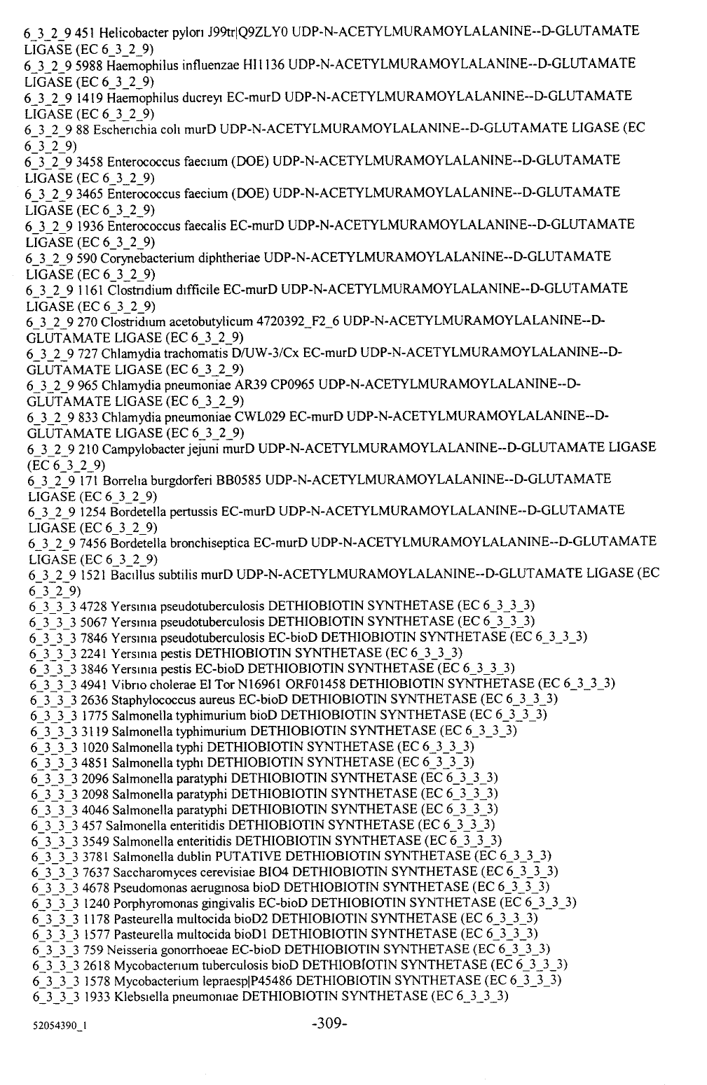

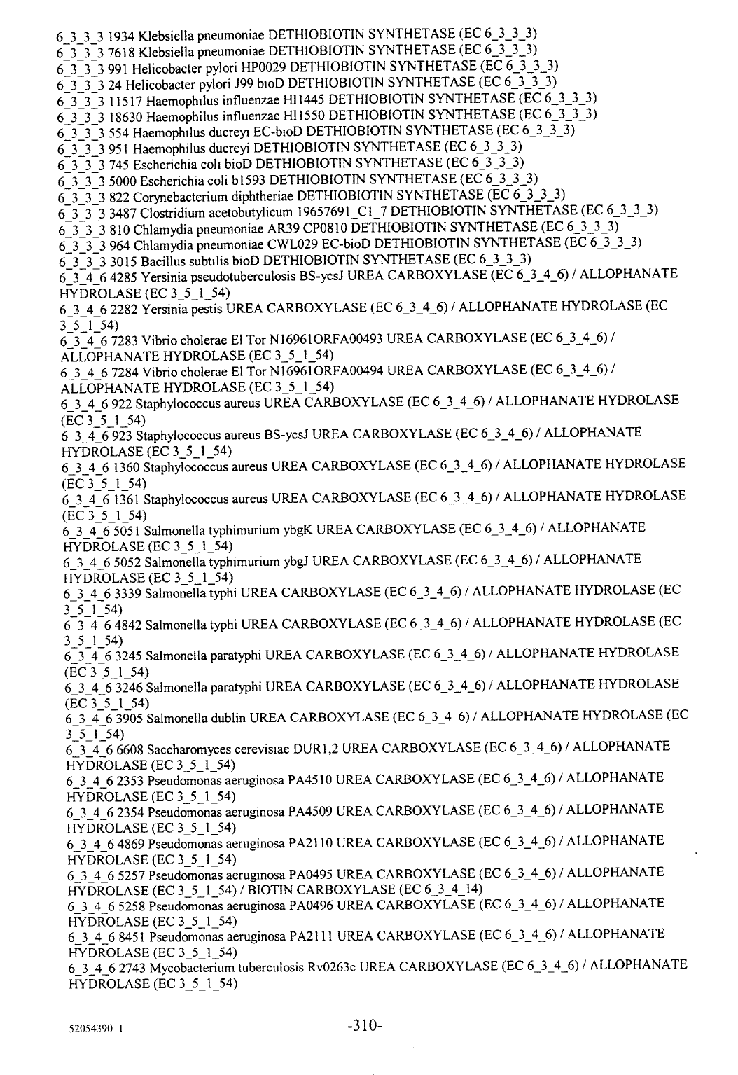

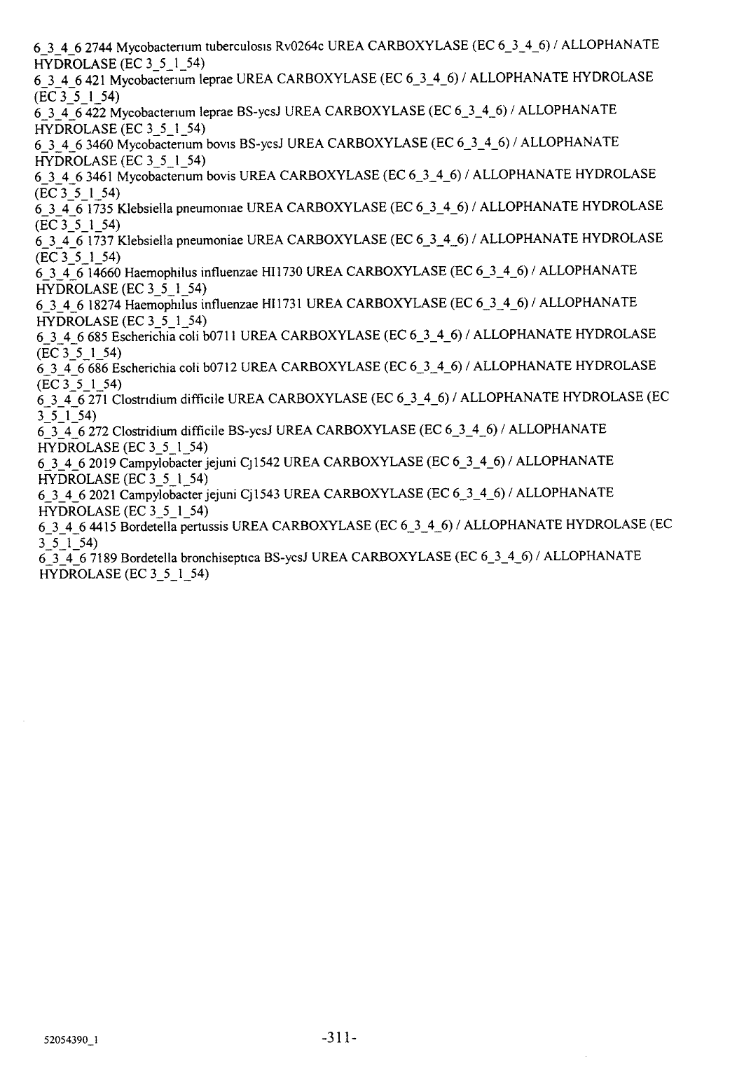

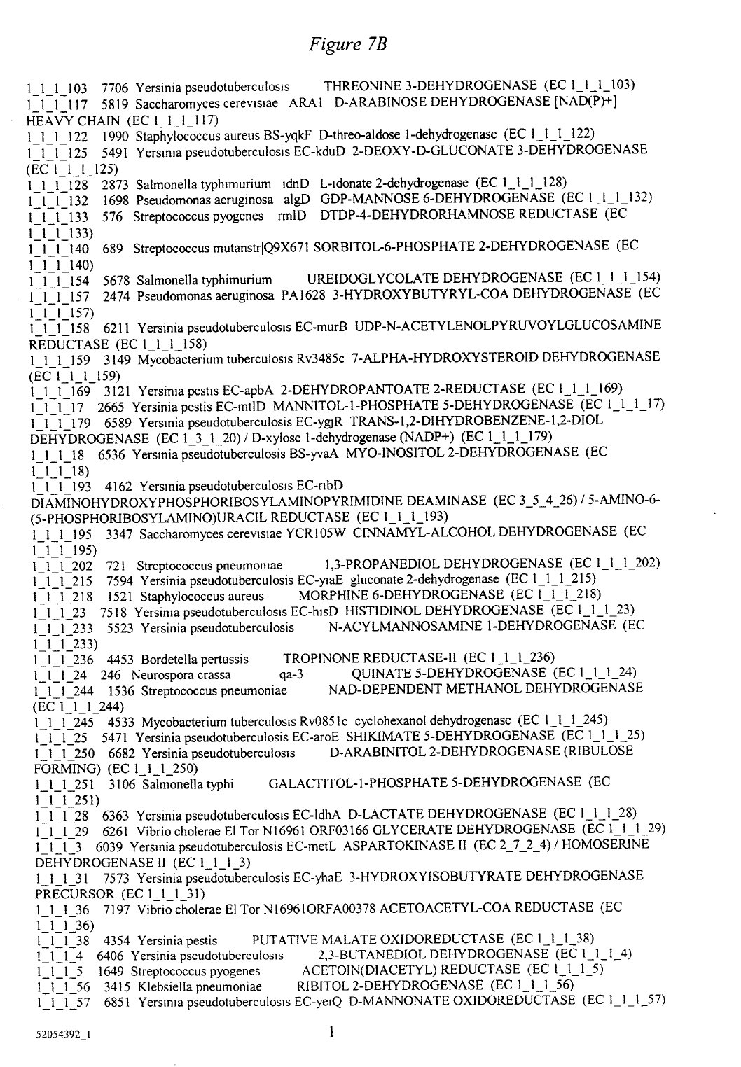

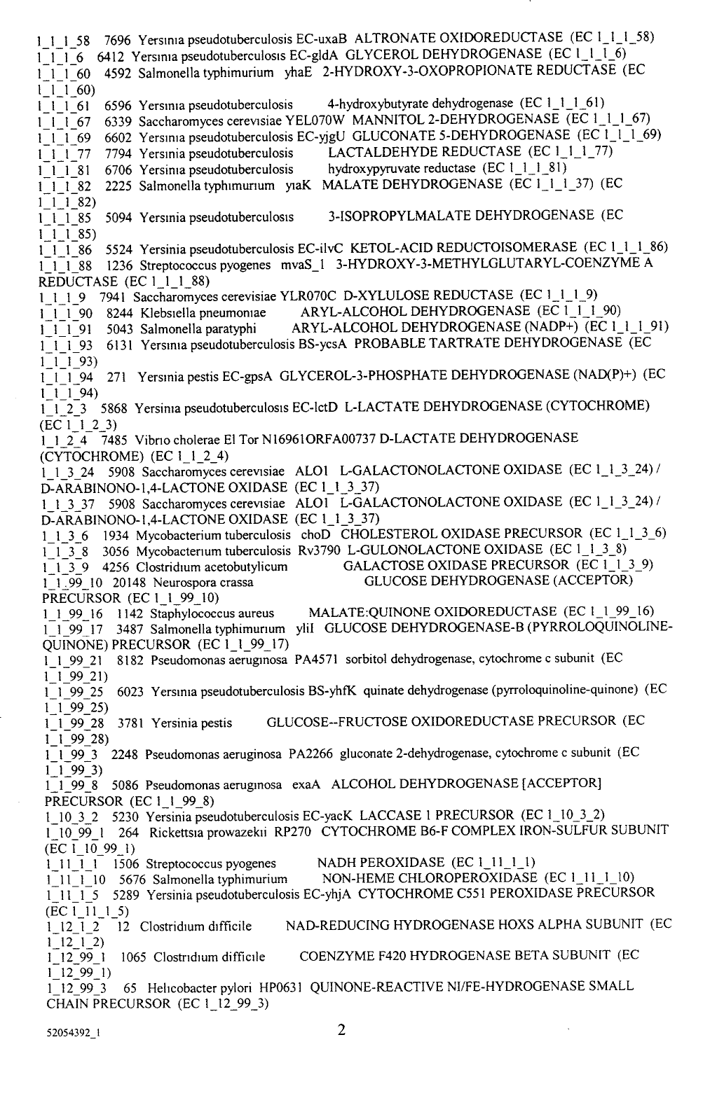

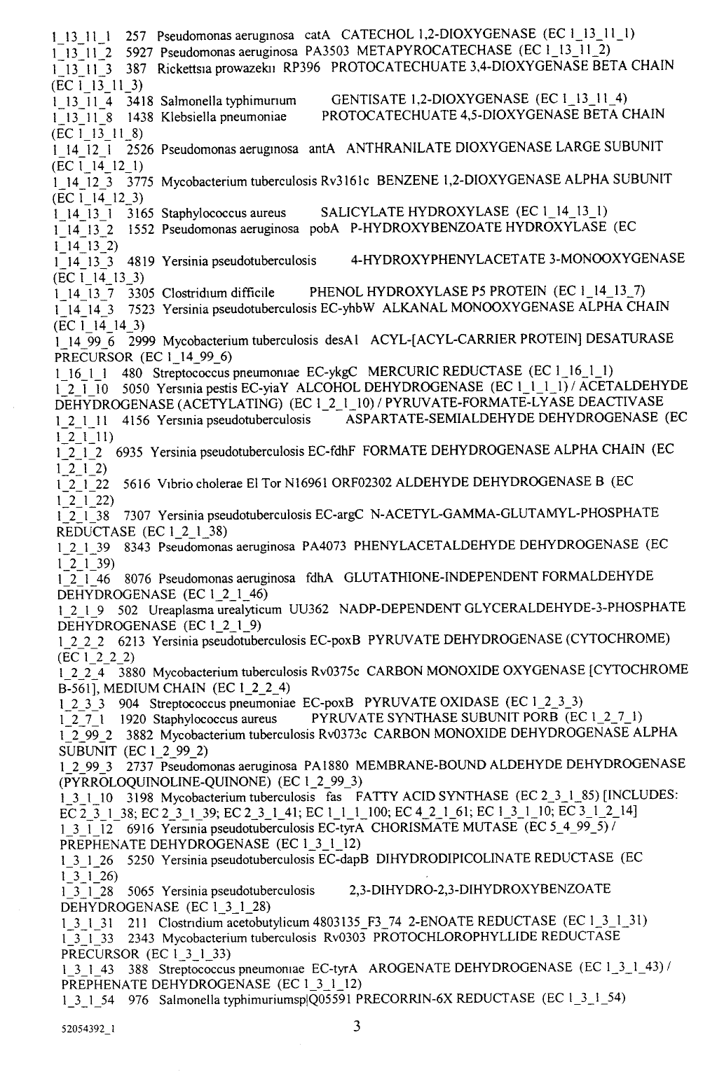

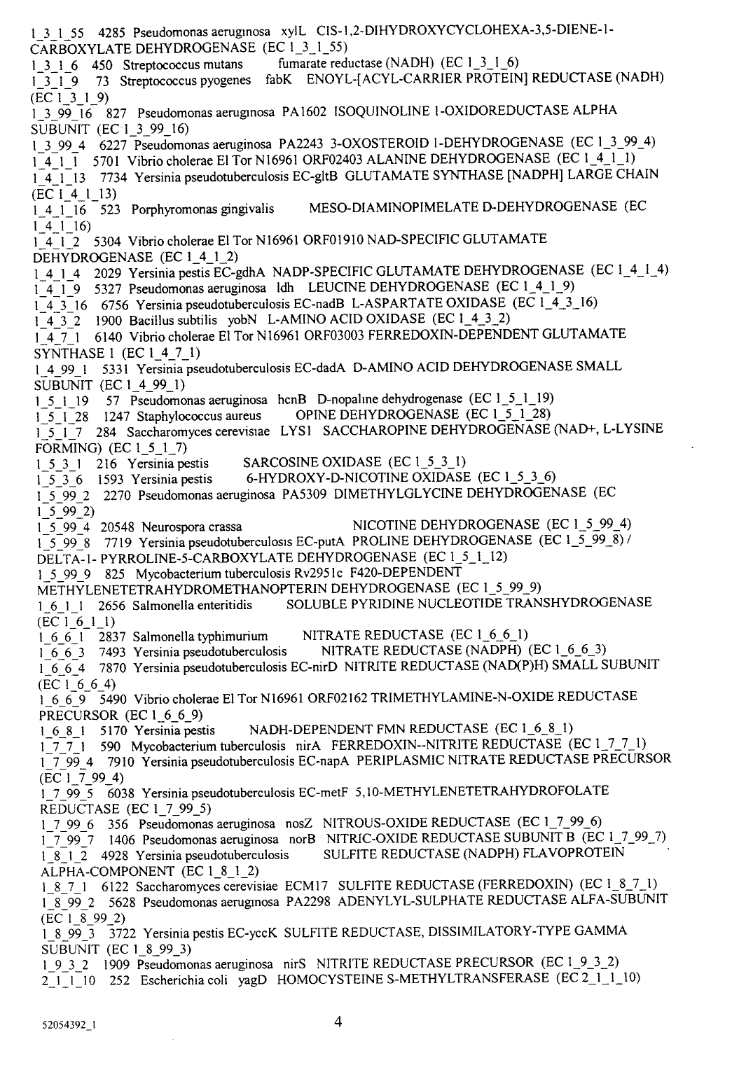

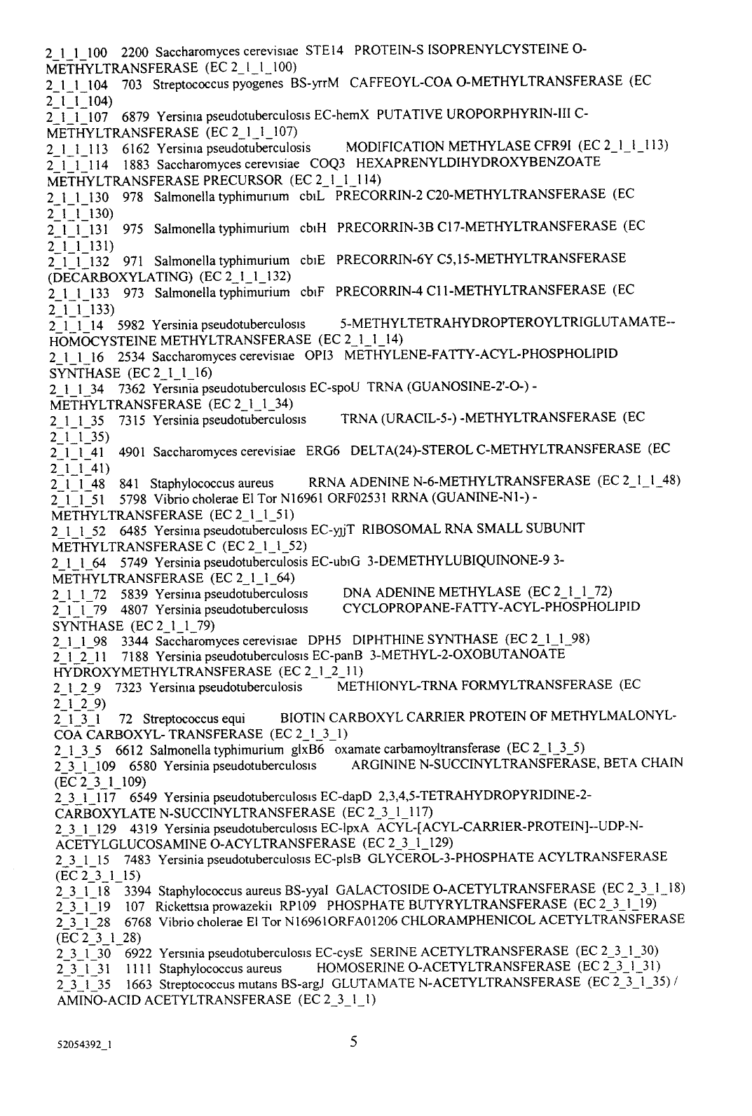

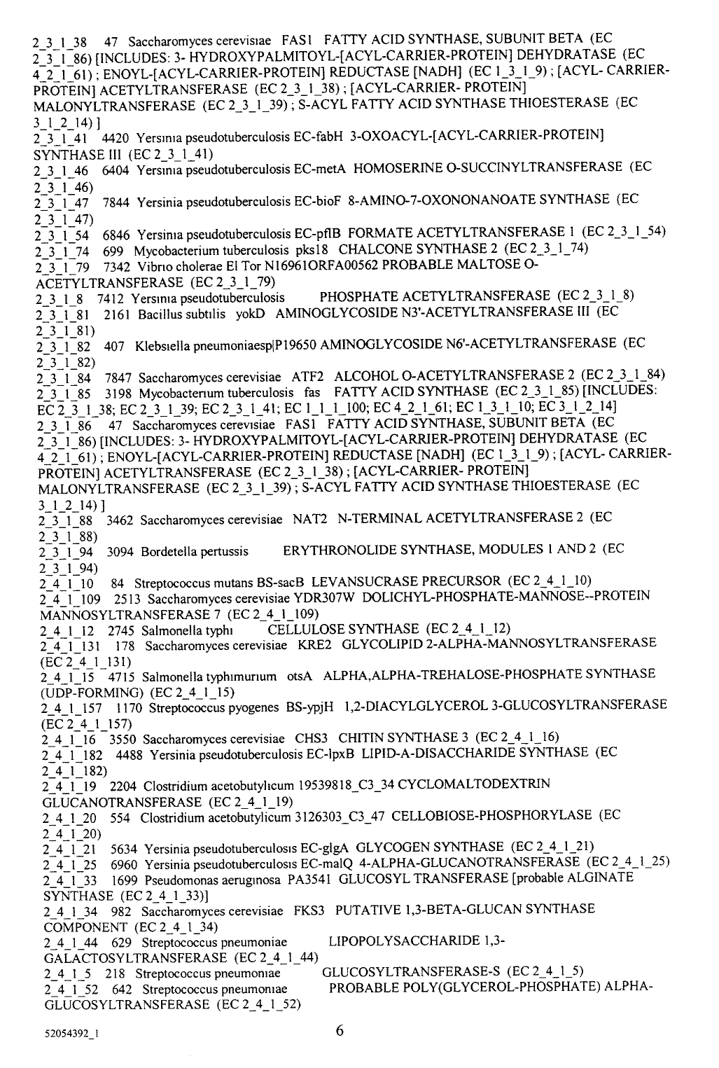

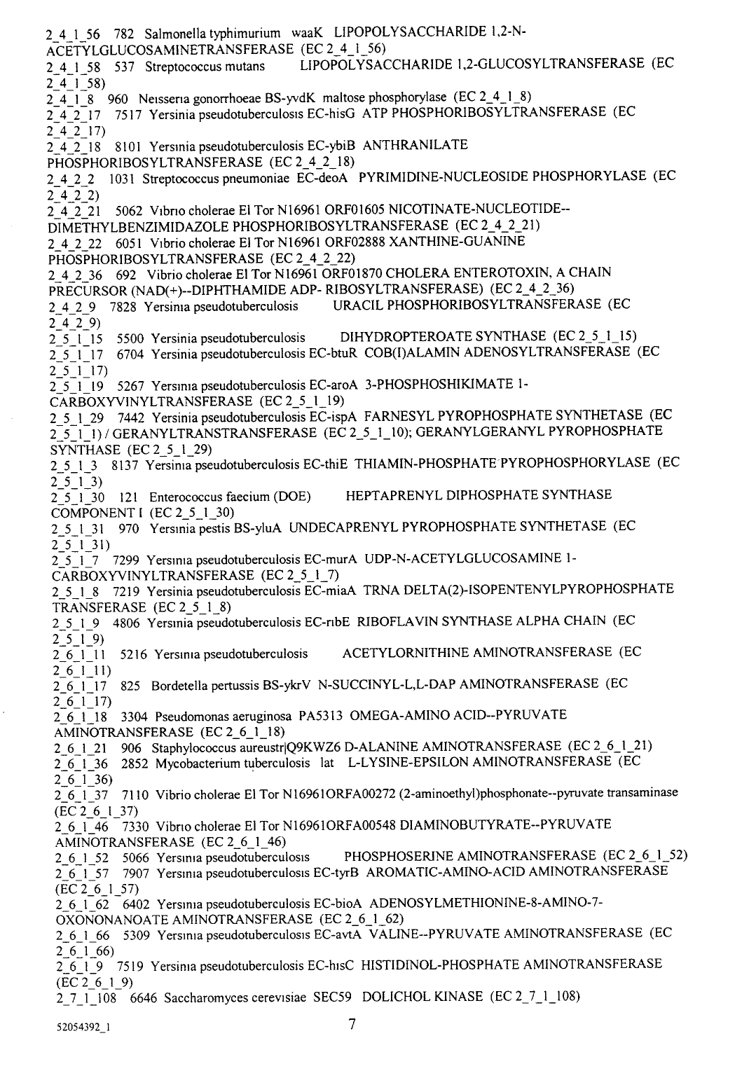

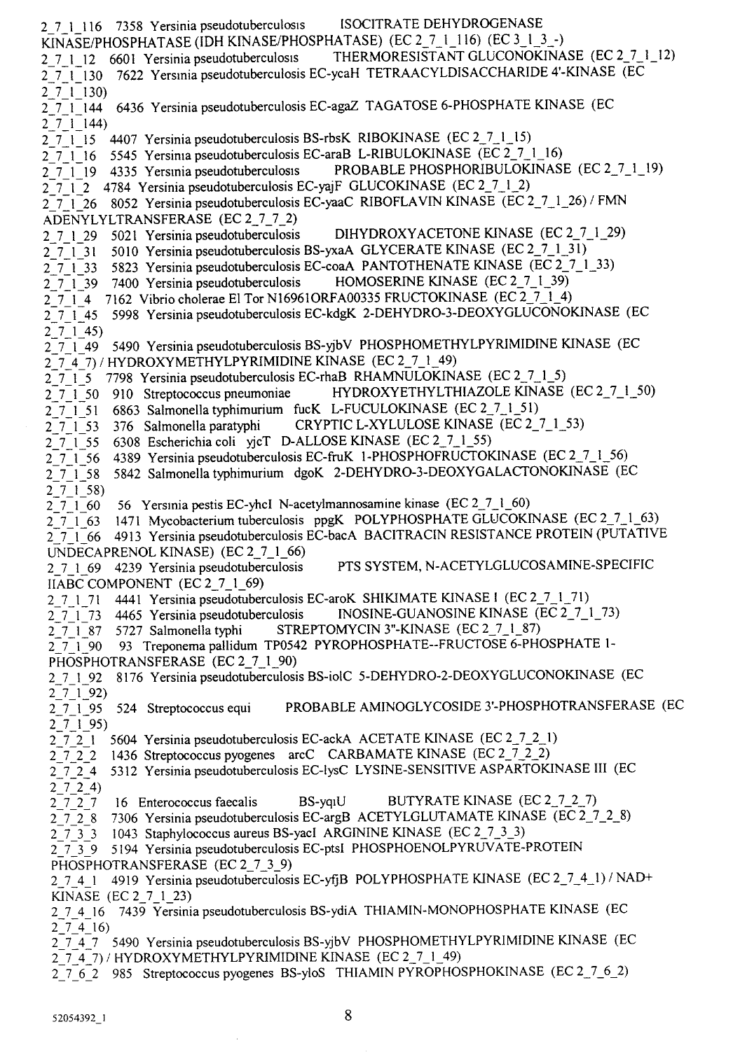

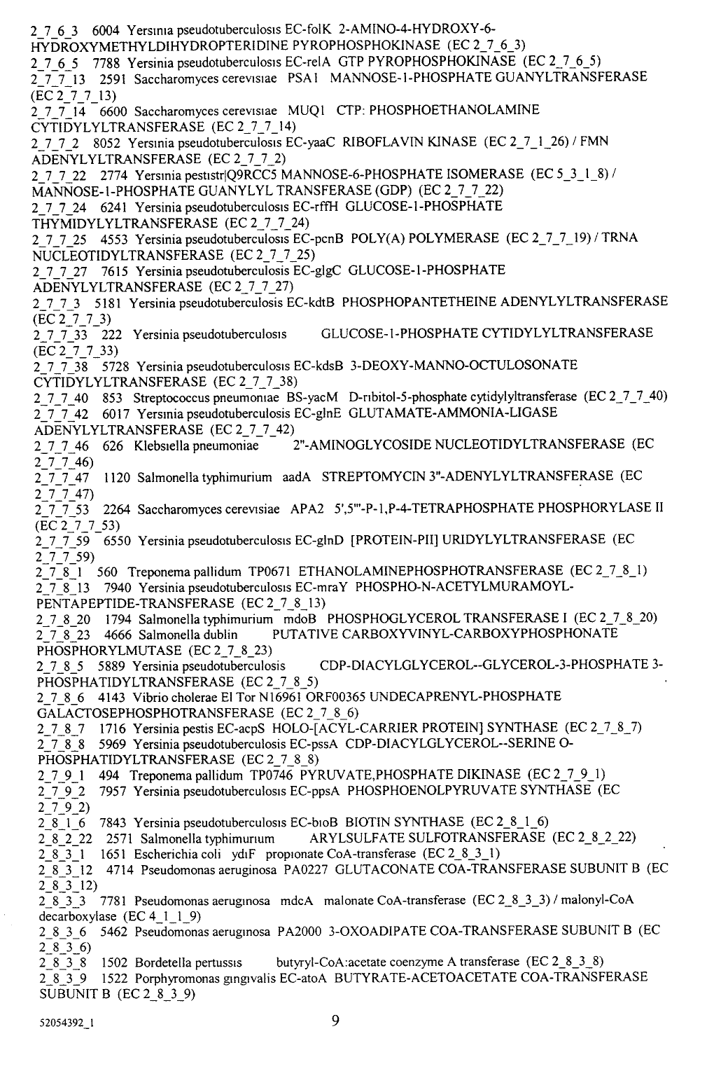

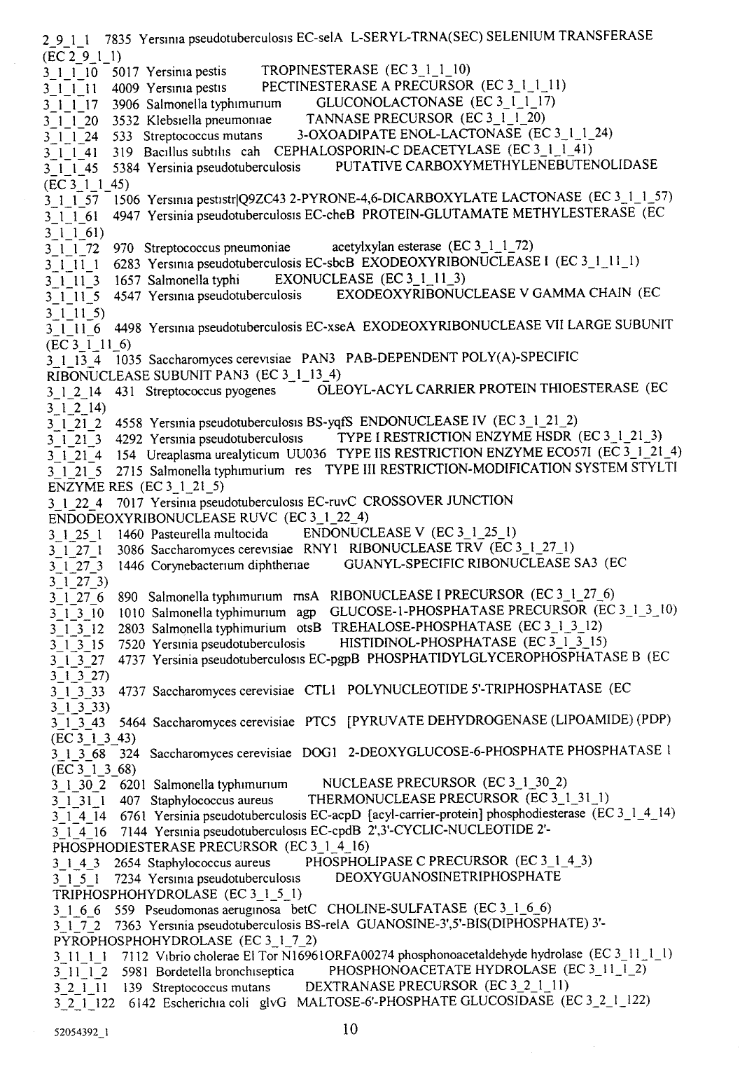

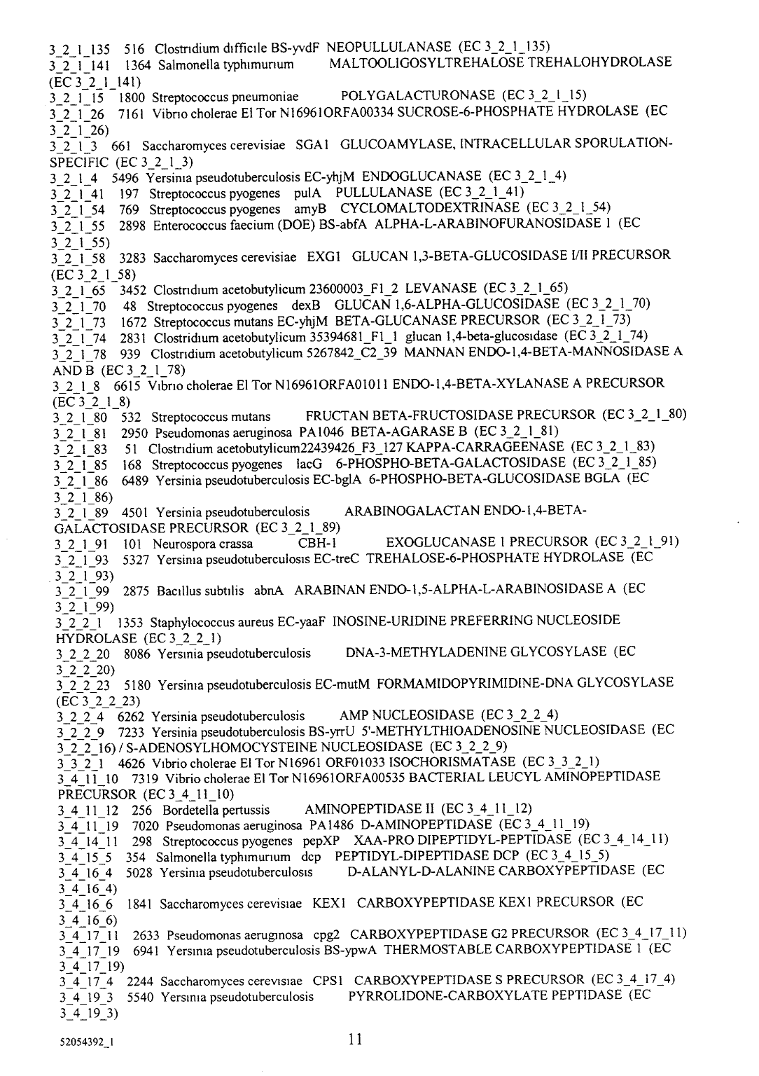

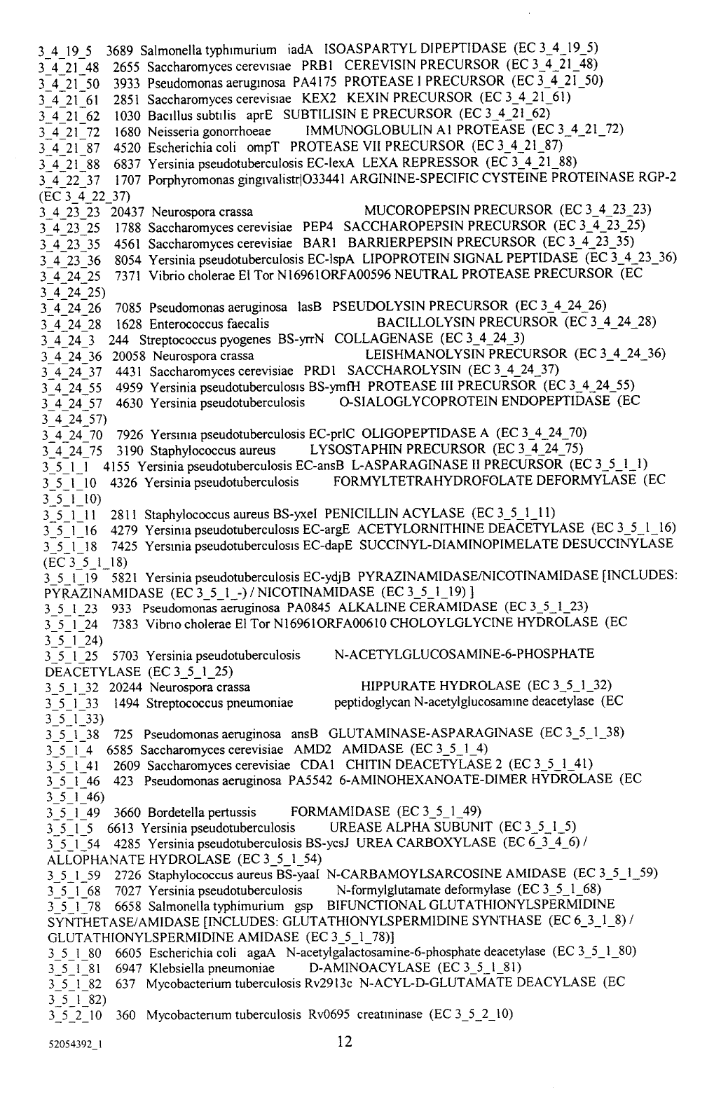

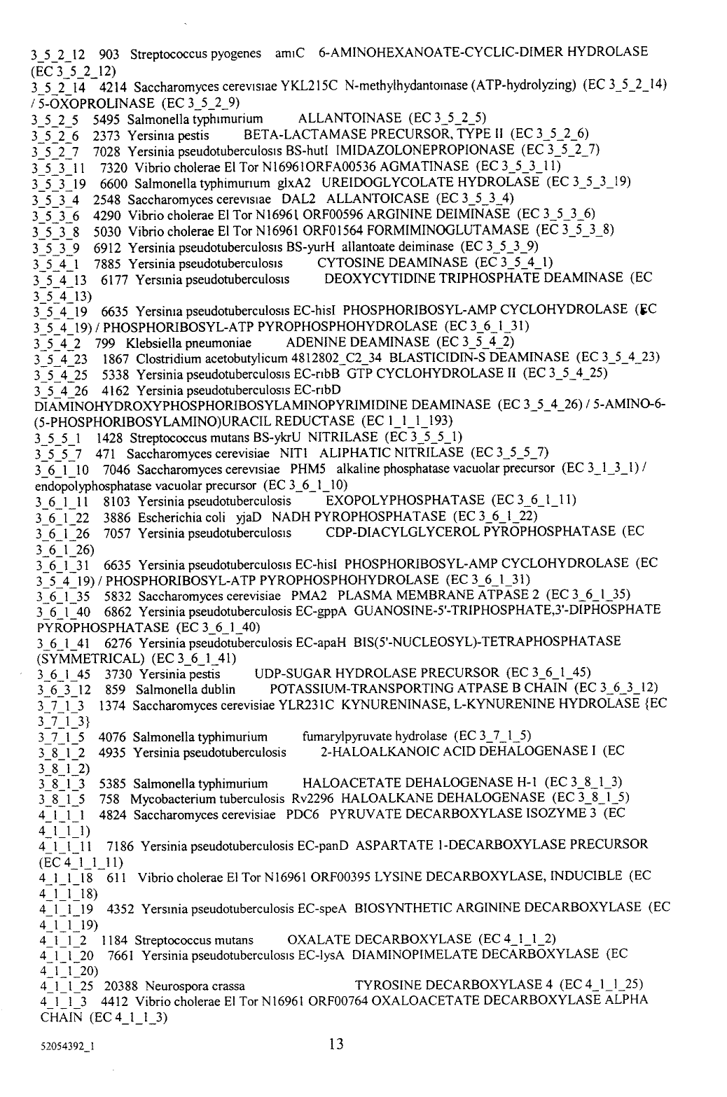

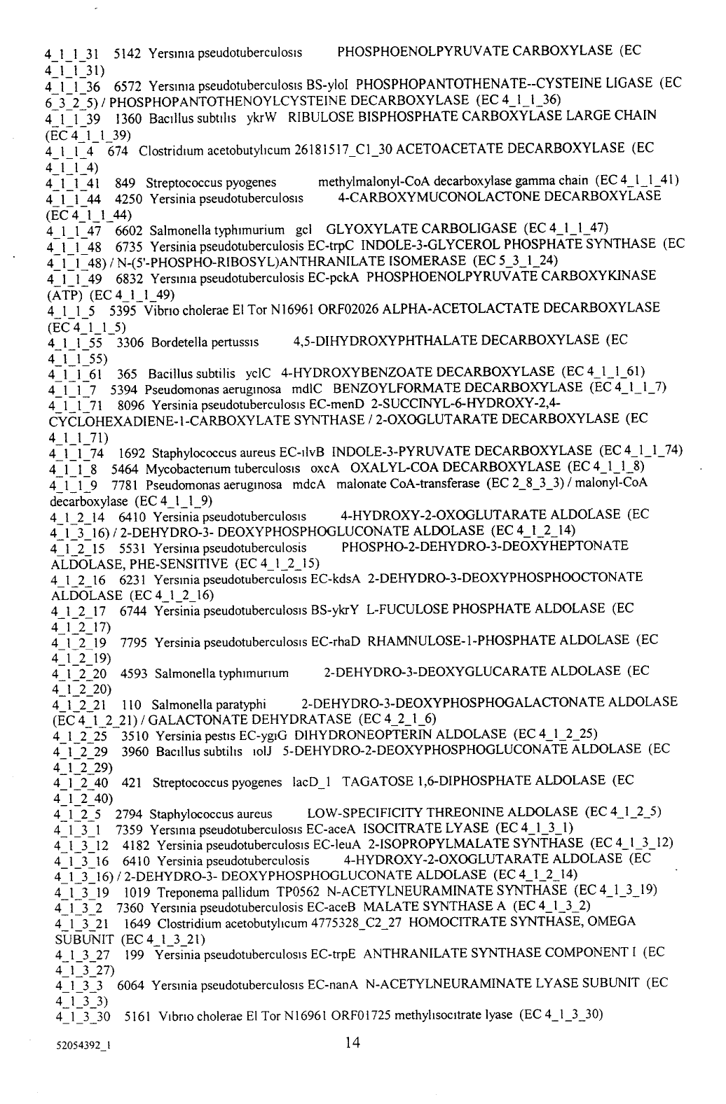

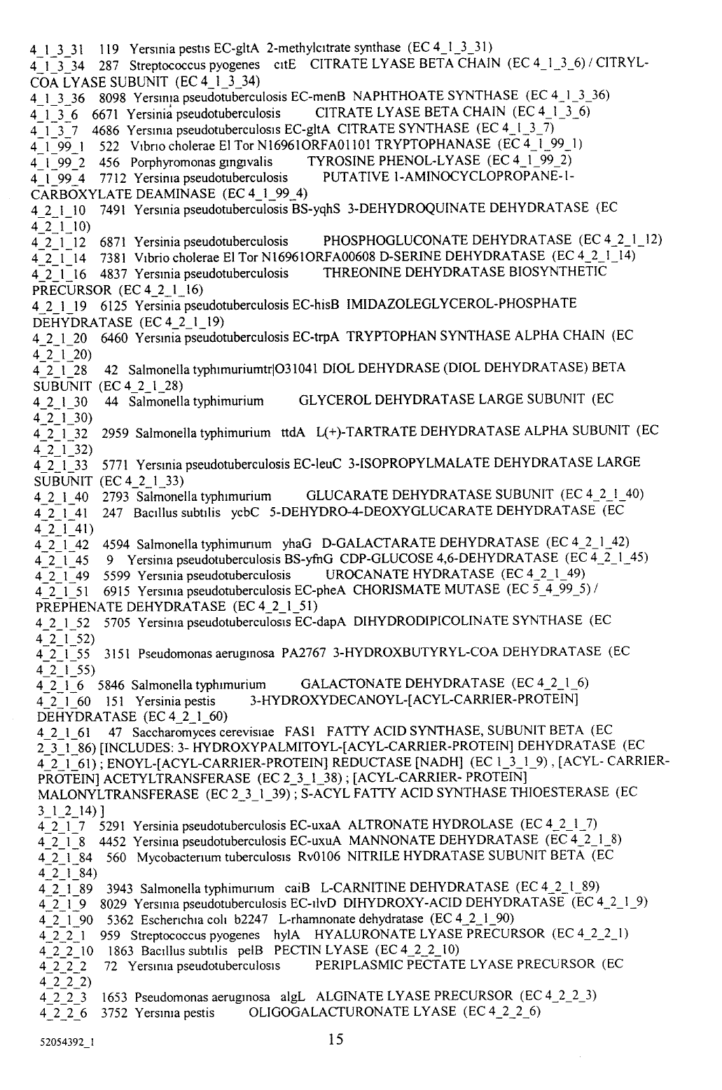

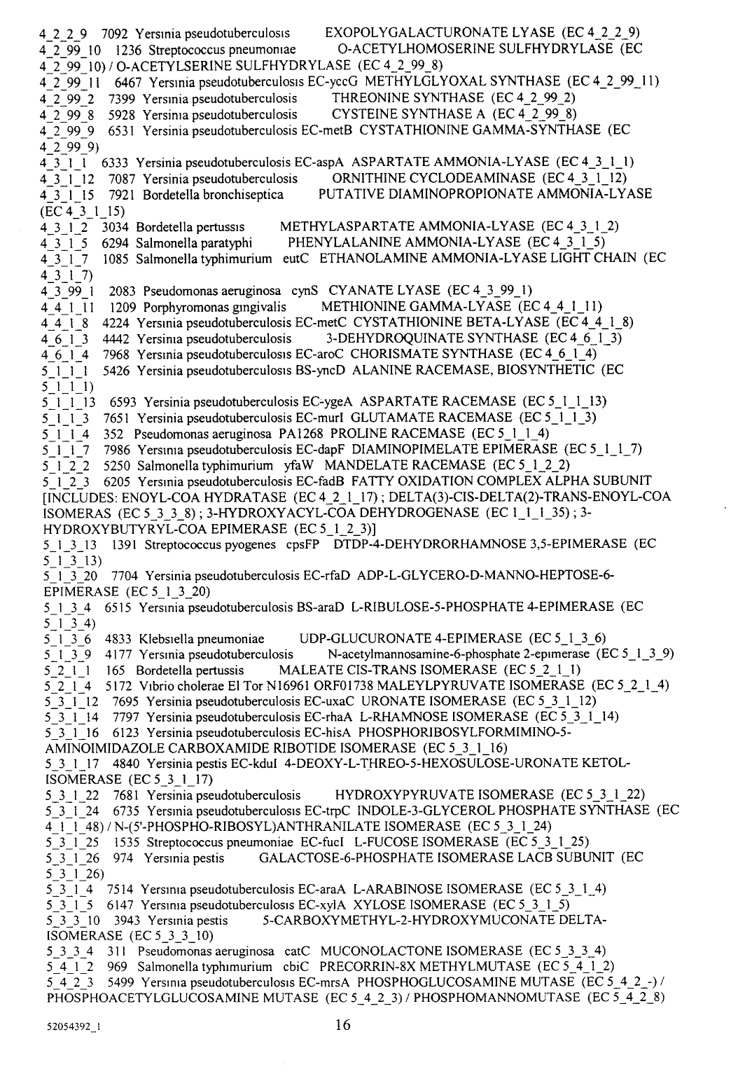

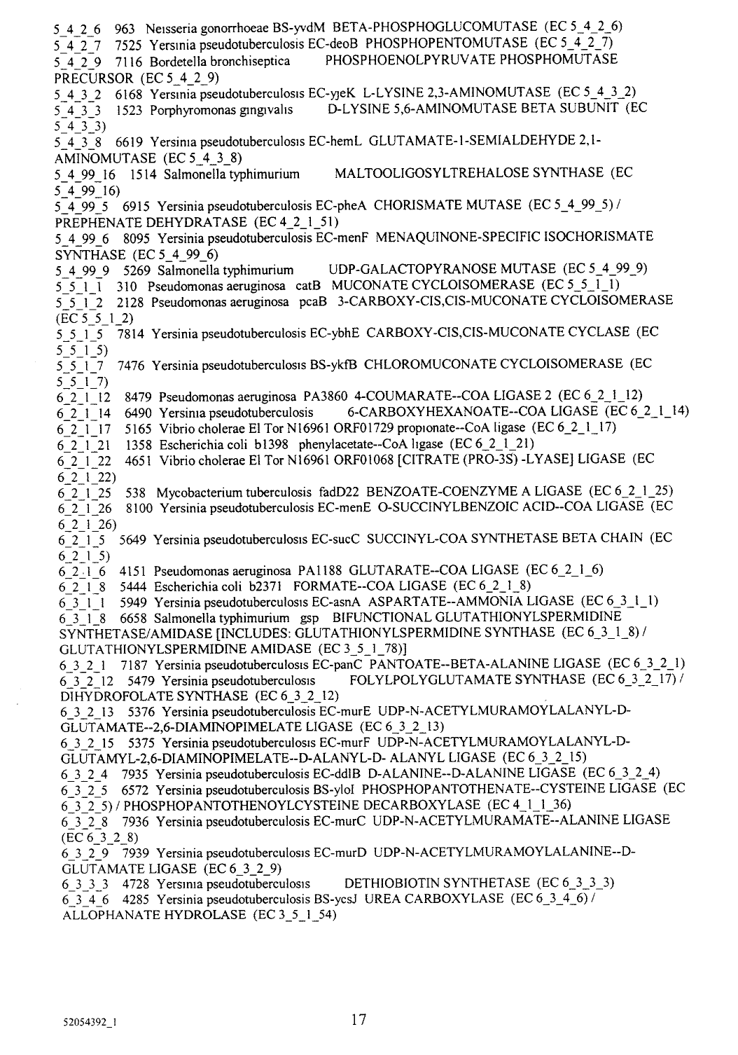

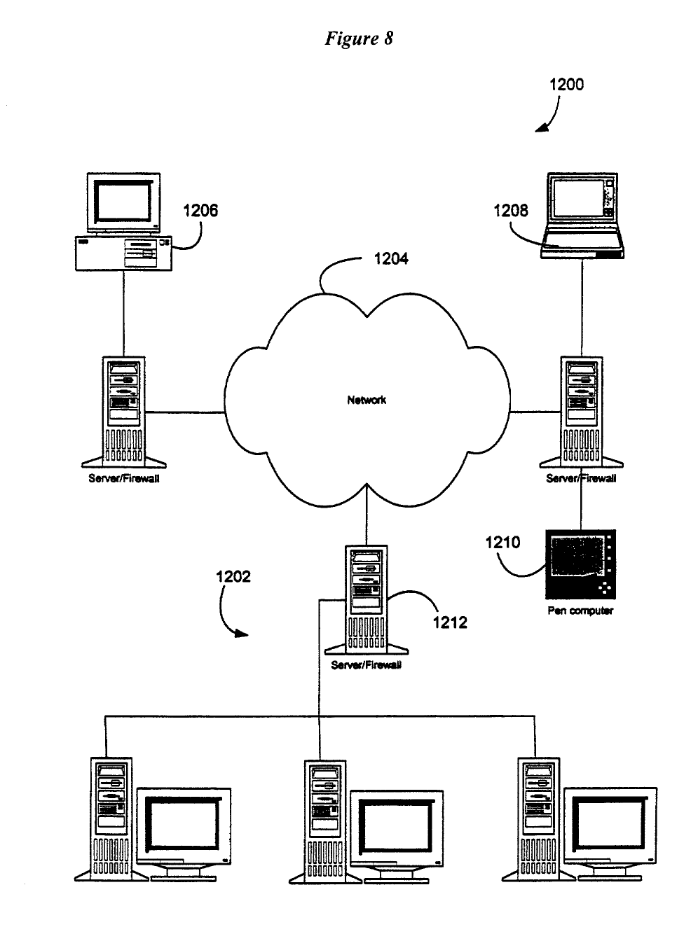

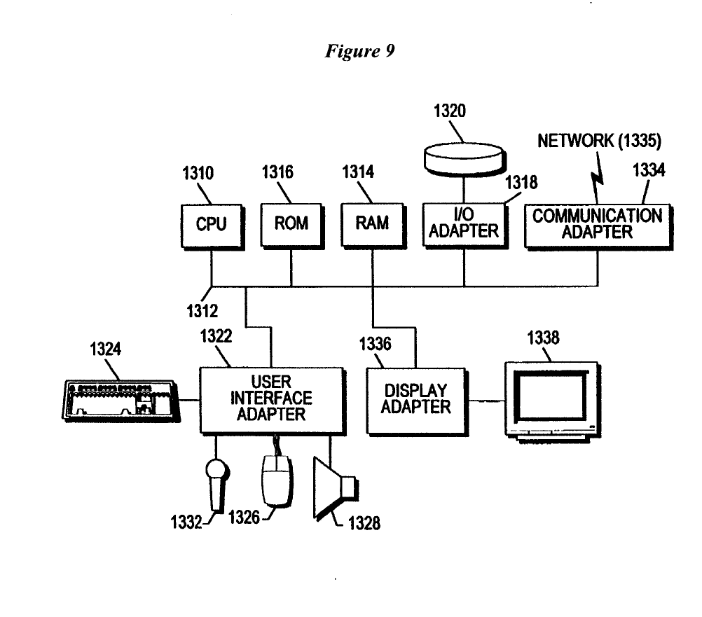

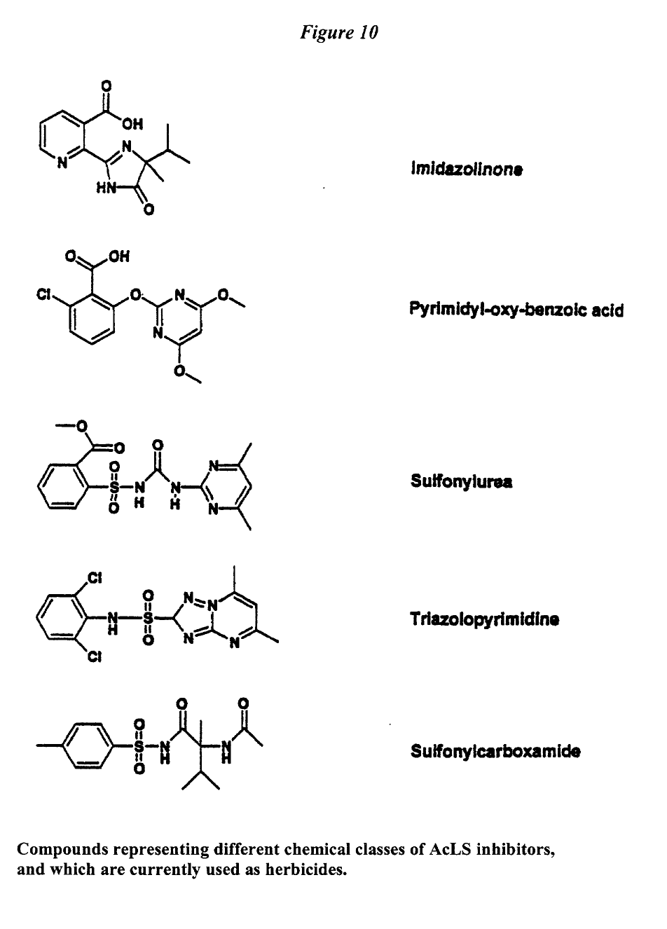

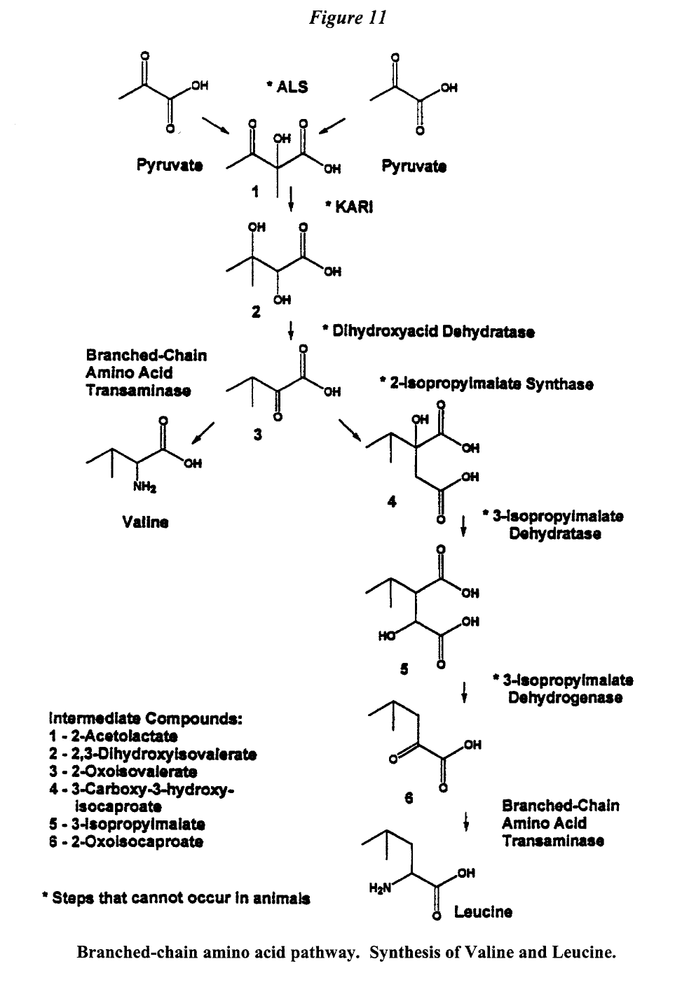

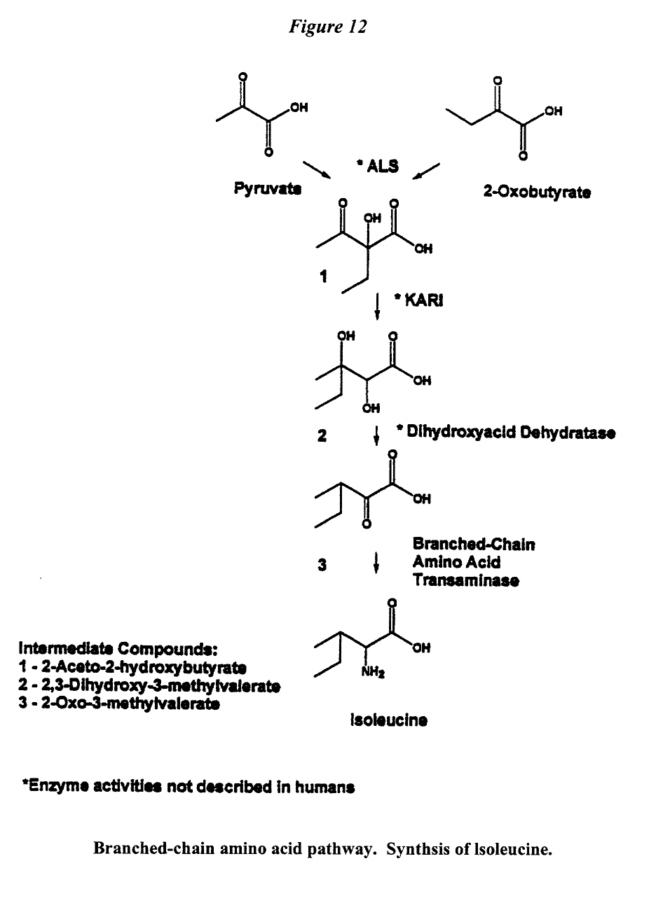

FIG. 7B is an abbreviated list consisting of all the EC number descriptions, but listing only one occurrence for each organism and consists of the 673 enzymes.

FIG. 8 illustrates an illustrative system with a plurality of components in accordance with one embodiment of the present invention.

FIG. 9 illustrates a representative hardware environment in accordance with one embodiment of the present invention.

FIG. 10 shows chemical structures representative of different chemical classes of AcLS inhibitors currently used as herbicides.

FIG. 11 shows the synthetic pathway for valine and leucine.

FIG. 12 shows the synthetic pathway for isoleucine.

### MODES FOR CARRYING OUT THE INVENTION

The practice of the present invention will employ, unless otherwise indicated, conventional techniques of modem biology and chemistry, including but not limited to molecular biology, biochemistry, microbiology, cell biology, enzymology, organic synthesis, medicinal chemistry, which are within the skill of the art. See, e.g., Sambrook, et al. Molecular Cloning: A Laboratory Manual, 2^{nd} edition (1989); Current Protocols In Molecular Biology (F. M. Ausubel, et al. eds., (1987)); the series Methods In Enzymology (Academic Press, Inc.): PCR 2: A Practical Approach (M. J. MacPherson, B. D. Hames and G. R. Taylor eds. (1995)); Animal Cell Culture (R. I. Freshney, ed. (1987)); and J. March, Advanced Organic Chemistry: Reactions, Mechanisms And Structure, 4^{th} edition (John Wiley & Sons, NY (1992)).

As used in the specification and claims, the singular form “a”, “an” and “the” include plural references unless the context clearly dictates otherwise. For example, the term “a cell” includes a plurality of cells, including mixtures thereof.

The term “comprising” is intended to mean that the compositions and methods include the recited elements, but do not exclude others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

An “infectious agent” is intended to be synonymous with “pathogen” and includes, but is not limited to bacteria, parasites, rickettesia, virus, and fungus.

Any of the terms “toxin”, “toxoid”, “prototoxophore”, “toxophore”, “Tox”, or “TOX” are synonymous and intend any molecule or functional group that is released or unmasked (revealed) upon the action of the enzyme resulting in toxicity to the pathogen, pathological cell or in an infected host cell. As apparent to those of skill in the art, the toxin or toxoid will vary with the target enzyme, the pathogen, the host cell and the subject being treated. Examples of toxins include, but are not limited to anthracyclins, vinca alkaloids, mitomycins, bleomycins, penicillins, cephalosporins, oxacillins, carbopenems, tetracyclins, chloramphenicols, macrolides, cycloserines, fluoroquinolones, glycopeptides, aminoglycosides, peptide antibiotics, oxazolidinones, quinolones, sulfonamides, cytotoxic nucleosides, pteridine family, nitrogen mustards, polyhalogenated biphenyls, diynenes, podophillotoxins, taxoids, alkylating agents. Some of the useful representatives of these classes include doxorubicin, carminomycin, daunorubicin, aminopterin, methotrexate, methopterin, dichloromethotrexate, mitomycin C, porfiromycin, 5-fluorouracil, 6-mercaptopurine, cytosine arabinoside, podophillotoxin, etoposide, etoposide phosphate, melphalan, vindesine, vinblastine, vincristine, leurosidine, leurosine, bis-(2-chloroethyl)amine, trichlorcarban, trichlorocarbanilide, tribromosalicylanilide, sulphamethoxazole, chloramphenicol, cycloserine, trimethoprim, chlorhexidine, hexachlorophene, fentichlor, 5-chloro-2-(2,4-dichlorophenoxy)phenol, 4-chloro-2-(2,4-dichlorophenoxy)phenol, 3-chloro-2-(2,4-dichlorophenoxy)phenol, 6-chloro-2-(2,4-dichlorophenoxy)phenol, 5-chloro-2-(3,4-dichlorophenoxy)phenol, 5-chloro-2-(2,5-dichlorophenoxy)phenol, 5-chloro-2-(3,5-dichlorophenoxy)phenol, 2,2′-dihydroxy biphenyl ether, halogeneted 2-hydroxybenzophenones, 2-mercaptopyridine-N-oxide, combretastatin, camptothesin, apoptolidene, cisplatin, epothilone, halichondrin, hemiasterlin, methioprim, thapsigargin, chloroquine, 4-hydroxycyclophosphamide, etoposide, colchicine, melphalan, quercetin, genistein, erbstatin, N-(4-aminobutyl)-5-chloro-2-naphtalen-sulfonamide, pyridinyloxazol-2-one, isoquinolyloxazolone-2-one, verapamil, quinine, quinidine, chloroquine, 2-halo ketones, nitrosoureas and reactive byproducts, epoxides, bromonium ions, aziridinium ions. Functional groups that are unmasked or revealed include the conversion of vinyl halides to allyl halides as in NB1011 (discussed infra).

A “prodrug” is s a precursor or derivative form of a pharmaceutically active agent or substance that is less cytotoxic to target or hyperproliferative cells as compared to the drug metabolite and is capable of being enzymatically activated or converted into the more active form (see Connors, T. A. (1986) and Connors, T. A. (1996)). The toxicity of the agent is directed to cells that are producing the converting enzyme in an amount effective to produce a therapeutic concentration of the cellular toxin in the diseased cell.

A “composition” is intended to mean a combination of active agent and another compound or composition, inert (for example, a detectable agent or label or a pharmaceutically acceptable carrier) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, Remington's Pharm. Sci., 15th Ed. (Mack Publ. Co., Easton (1975)).

An “effective amount” is an amount sufficient to effect beneficial or desired results. An effective amount can be administered in one or more administrations, applications or dosages. The term “effective amount” is to include therapeutically or prophylactically effective amounts. Thus, the term also refers to an amount effective in treating or preventing an infection in a patient or an infestation in a plant either as monotherapy or in combination with other agents.

The term “prophylactically effective amount” refers to an amount effective in preventing infection in a subject or plant infestation.

The term “linker” indicates a spacer or connector between two parts of a single molecule such that when a particular bond is severed between the two parts of the molecule separate.

“Inhibiting the growth” of a microorganism or infected cell means reducing by contact with an agent, the rate of proliferation of such a microorganism or infected cell, in comparison with a control microorganism of the same species not contacted with this agent or as compared to an uninfected cell.

The term “treating” refers to any of the following: the alleviation of symptoms of a particular disorder in a patient; the improvement of an ascertainable measurement associated with a particular disorder; or a reduction in microbial number. One of skill in the art can determine when a host has been “treated” by noting a reduction in microbial load or an alleviation in symptoms associated with infection.

A “subject,” “individual” or “patient” or “host” is used interchangeably herein and refers to plants, avians, fish and animals, e.g., a vertebrate, preferably a mammal, more preferably a human. Mammals include, but are not limited to, murines, simians, humans, farm animals, sport animals, and pets.

A “control” is an alternative subject or sample used in an experiment for comparison purpose. A control can be “positive” or “negative”. As known to those of skill in the art, a “suitable control” is variable and depends in part on one or more of the following criteria: the target pathogen, the target enzyme, expression level of the target enzyme, the host cell, the subject or host as well as the specific genotype or phenotype of each. For example, when the object of the method is to identify target enzymes in pathological cells such as cancer, a suitable control can be one or more of a normal counterpart cell, a counterpart cancer cell that has not undergone any therapy or been exposed to an inducing agent or a different therapy or a cell that has been treated in a different environment or microenvironment, e.g., in vitro versus in vivo. Alternatively, the control cell can be one that been or will be treated with a known therapeutic or therapeutic method. When the object of the invention is to identify intrinsic ECTA target enzymes expressed by pathogens or in pathogen-infected cells, the control counterpart can be one or more of a pathogen that has not been exposed to an inducing agent or one that is not infected with the pathogen.

An “inducing agent” includes any agent (chemical, physical or mechanical) which alters the genotype or phenotype of a pathological cell or infectious agent or infected cell. Examples include prior chemotherapy (in the case of cancer), prior treatment with one or more antibiotics (in the case of pathogens and pathogen-infected cells) or prior exposure to another organism resulting in the exchange of genetic material, e.g. plasmids that confer antibiotic resistance to a host cell. Additional examples include, but are not limited to exposure to radiation, chemicals, ultra-violet light, metals or genetic manipulation.

As used herein, “expressed at elevated levels” in the context of infectious disease, is intended to include any amount over the base line or control as compared with host cells (e.g., an uninfected or normal cell) taking into consideration the sensitivity of the detection system and statistical variation in the computation methods. In the context of cancer, “expressed at elevated levels” is intended to include any amount that is more than an amount over the base line or control (e.g., a normal counterpart cell) taking into consideration the sensitivity of the detection system and statistical variation in the computation methods. In some aspect, it is at least 2×, or more than 3× or preferably more than 4× than that expressed in a normal cell.

A “favorable reaction type” as used herein, refers to a chemical reaction catalyzed by an enzyme wherein an enzyme that catalyzes such a reaction has been shown to be effective at metabolizing ECTA substrates.

As used herein, the terms “pathological cells, “target cells”, “host cells” and “hyperproliferative cells” in the context of cancer, encompass cells characterized by the activation by genetic mutation or the endogenous overexpression of an intracellular enzyme which may confer resistance to the inhibitory or cytotoxic effects of chemotherapy. Overexpression of the enzyme can be related to loss of tumor suppressor gene product function drug resistance or the genetic instability associated with a pathological phenotype. In the context of infectious agents, the terms encompass cells infected with or containing an infectious agent as defined herein. In the context of cells and infectious agents showing resistance to antibiotics, the terms encompass cells overexpressing an enzyme which confers resistance to the cytotoxic effects of the antibiotic.

“Sequence comparison” is used to compare character strings representing proteins or fragments of DNA to gather evidence for common function or biological origin. Proteins which are thought to have a common ancestor are called homologous. The process of evolution introduces mutations in DNA which may take the form of: the substitution of one or more nucleic acids for another; the deletion of one or more nucleic acids; or the insertion of one or more nucleic acids.

These changes in the genetic material of organisms can lead to corresponding changes in the amino acid sequences for the corresponding proteins. Close “homologs” tend to share substantial portions of their amino acid sequences, and so sequence comparison algorithms are used as a tool to detect homologies. It is, however, by no means a foolproof tool: there are examples of proteins that have substantial sequence similarity but serve very different functions and exhibit different three-dimensional structures, and so are probably not homologs. On the other hand, there are proteins with very little sequence similarity but which nonetheless have similar functions and three-dimensional structures, and are considered homologs.

A biological sequence is a finite string of characters drawn from some alphabet. Typically these strings will represent amino acid sequences (proteins; alphabet size=20) or nucleic acid sequences (DNA; alphabet size−4). We write s[i] for the ith character of s, where i is between 1 and |s|, the length of s.

An “alignment” of two strings s and t on the same alphabet A is a pair of strings s′ and t′ on the alphabet A+{‘−’}=: A′, where ‘−’ is a special character not in A that represents a “gap” or “space”, such that

|s′|=|t′|;

removing all the ‘−’ characters from s′ leaves s, and similarly for t′ and t; and

gaps are never paired with gaps; that is, if s′[i]=‘−’ then we do not have t′[i]=‘−’, and vice-versa.

The goal of pairwise sequence alignment algorithms is to find a high-scoring alignment of a given pair of sequences (or subsequences of those sequences), according to some prescribed alignment scoring method.

“Classic dynamic programming algorithms” are thought of as exact, in the sense that they are guaranteed to compute the best possible alignment of the two strings under the supported alignment scoring system. The scoring systems usually prescribes a method of scoring character pairings, and the total score for a particular alignment is the sum of the character pair scores. A scoring function S: A′×A′→R is symmetric in its two arguments. Typically, character matches are awarded positive scores, and mismatches may be assigned different scores depending on the severity of the mismatch (for example, two amino acids that have similar chemical properties may be substituted for one another without greatly affecting the function of the resulting protein, and matching one with the other may be almost as good as a perfect match and be awarded a positive score. On the other hand, two amino acids may have very different chemical properties and their mismatch may be awarded a negative score). Gaps are usually awarded negative scores. In the simplest case, the penalty for a gap in the completed alignment is proportional to its length, and the scoring function may be represented as a symmetric matrix. However, there are biological reasons for penalizing small gaps more heavily than larger ones, and popular implementations usually use affine gap penalties of the form I+R*(k−1) for a gap of length k; this requires a minor change in representation for the scoring function.

These algorithms proceed by constructing a (|t|+1)*(|s|+1) matrix M of partial alignment scores, frequently called the “dynamic programming matrix”. M[i, j] is interpreted as the score of the best alignment of the subsequences t[l . . . i] and s[l . . . j] that ends by pairing t[i] with s[j]. The zeroth column and row represent leading gaps, and are assigned negative scores according to the gap scoring regimen. The fundamental notion in all these algorithms is that the value of M[i, j] must be the best (maximum) of

M[i, j−1]+S[‘−’,j] pair a gap in t′ with S[j]

M[i−1,j−1]+S[i, j] pair t[i] with s[j]

M[i−1,j]+S[i,‘−’] pair t[i] with a gap in s′

assuming that M[i-1,j], M[i−1,j−1] and M[i, j−1] are all previously computed.

A highest-scoring alignment can then be recovered from the matrix M by using a “traceback procedure”. Tracing back from element M[i, j] involves recomputing the scores for the extensions of prefix alignments as above, then selecting one that equals M[i, j], and then tracing back from the corresponding element M[i−1,j], M[i−1,j−1], or M[i, j−1] (Of course, it could be that two or all three of the prefix alignments lead to the same score; in this case, there is usually some policy on selecting one type of alignment over another. It is also possible to maintain a list of equivalent alignments and report all of the best-scoring alignments). The traceback starts at the maximum element in the last row together with the last column of M, and ends when the zeroth row or column is reached.

The algorithm described above is similar to the Needleman-Wunsch global alignment algorithm. The algorithm is called a “global” alignment algorithm because it tries to find the best alignment over the whole strings s and t. With a slight change, the same technique can be used to find the best local alignment between s and t, that is, the highest-scoring (global) alignment of substrings s[i₁ . . . i₂] and t[j₁ . . . j₂]. The changes required are:

the zeroth row and column are initialized with zeros;

in the dynamic programming computation, let M[i, j] be the maximum of the given quantities above together with zero.

in the traceback procedure, start at the maximum element for the entire dynamic programming matrix, and stop as soon as an M[i, j]=0 is encountered.

This is the Smith-Waterman local alignment algorithm. Of the classic dynamic programming methods, it is the most commonly used.

For biological reasons one may wish to not penalize gaps that occur at the beginning or end of an alignment. These variations are easily accommodated by changing the initialization of the zeroth row and column of M.

The classic algorithm as presented requires O(|s|*|t|) (quadratic) time and space. The matrix M is normally filled in row-by-row or column-by-column, and it is never necessary to have more than two rows or columns of the matrix in memory at once. In many applications, the actual alignment may not be necessary, and only the maximum score over all possible alignments may be required. In this case, we need not store M but instead store only those rows (columns) necessary for the computation and the maximum value. In this case the algorithm uses only linear (O(min(|s|, |t|))) space.

It is however possible to recover the optimal alignment using only linear space, at the expense of doubling the computation time. The fundamental idea is to use a divide-and-conquer approach and recompute parts of the dynamic programming matrix (actually, maximum values over rectangular subregions of it) as required.

The “FAST” algorithm is a heuristic approach that tries to approximate the best (local) alignment and score while reducing the computational expense of the Smith-Waterman algorithm. Strictly speaking, it is a database search algorithm: we have a query string q, and wish to compare it against every string in a database of strings. Typically, it is best to report the best n scores and corresponding alignments, where n is much smaller than the database size. The computation for each database string s is a local alignment with q, and proceeds in four stages:

1) the strings are rapidly scanned for exact substring matches of length ktup. ktup is usually quite small, only 1 or 2. A table of |q|+|s|−1 counters is maintained, one for each diagonal in a table, similar to the dynamic programming matrix M; for a match of a k-tuple with starting points q[i] and s[j], the i-jth counter is incremented. At the end of this stage, the table of counters gives the number of hits for each diagonal.

2) for each diagonal with more than one hit, hits are merged into regions. These regions may contain mismatches, but since everything is in the same diagonal, there are no gaps.

3) the five best regions are rescored using a protein substitution matrix (for example, PAM120, PAM250, or, more recently, a BLOSUM matrix). The best of these scores is reported for the sequence pair, and is called the initial score. The matches in the database are ranked according to their initial scores.

4) the pairs with the n best initial scores are then re-examined using a modified Smith-Waterman alignment algorithm that is restricted to a band 64 diagonals wide centered around the best diagonal. This new score is called the optimized score. In reporting the final results, both initial and optimized scores are listed; often very good matches have a dramatically better optimized score than the initial score.

The FAST package includes a program for testing the statistical significance of high-scoring matches. It works by scrambling one of the strings and running the Smith-Waterman algorithm on the new pair; this is repeated many times. If the score reported for the original pair is sufficiently far from the mean score for the alignments on the scrambled strings, the match is considered significant (unlikely to be due to chance). Recent versions of FASTA evaluate the statistical significance of scores using a theory based on extreme value distributions.

Like FAST, BLAST (Basic Local Alignment Search Tool) is another heuristic database search algorithm that tries to reduce the time required to find good pairwise alignments. Like FAST, BLAST attempts to find high-scoring subsequences with no gaps, but its approach is a little different. Given a query protein sequence q and a database sequence s, BLAST:

1. examines the query sequence to find high-scoring substrings or words for matches. There are two parameters that affect the search for these words: a word length w and a threshold score T. The algorithm constructs the set of all w-length contiguous subsequences (w-mers) of the query q, and then for each w-mer d finds all possible w-mers that score at least T when compared with d using a protein substitution matrix (usually PAM, or, more recently, BLOSUM). Not all w-mers from q need contribute to the word list: if a word d scores less than T when compared with itself, it will not contribute at all;

2. scans the database using a hashtable or specially-constructed DFA for exact matches to entries in the word list (hits); and

3. extends hits. A hit is extended by adding characters to the front and back of each of the two substrings until a maximal score (under the same substitution matrix as above) is reached: dropping or adding a pair of characters at either end lessens the score. In practice, the pair is discarded if the score falls a prescribed distance below the best score reported for the same-length extension so far.

The best extension scores (or maximal segment pair (MSP) scores) are used to rank the database strings. The process for DNA is similar, except that the scoring is simpler (there are no substitution matrices), and the values of the parameters are different.

BLAST attempts to estimate the statistical significance of the MSP scores based on a statistical theory of how MSP scores should be distributed for random strings.

Identification of ECTA Target Enzymes—Methods and Systems

A method is provided that identifies ECTA enzymes. A first suitable data structure is searched to obtain a first set of information relating to one or more enzymes associated with a target organism. The enzyme can be one that is expressed, overexpressed or selectively expressed. This search provides a first enzyme list. A search also is conducted on one or more other suitable data structures to obtain one or more additional sets of information relating to one or more expressed enzymes associated with one or more controls. The first set of information is compared to the one or more additional sets of information to identify enzymes in the first set of information that are not present (or absent) in the first search, but not the second. These identified enzymes are targets for ECTA compounds.

Examples of data structures include, but are not limited to databases of genetic or expressed genetic information relating to enzymes. The information may be in the form of DNA, RNA or protein and may include, where appropriate information relating to quantitative expression of the enzyme. The information may be organized in any manner. In one aspect, the information is restricted to the pathogen or host cell expressing it. In another aspect, the information is organized by tissue distribution, e.g., enzymes expressed in cancer cells, enzymes expressed in normal, non-cancerous cells, enzymes overexpressed as a result of prior therapy (e.g., antibiotic or chemotherapy), or enzymes expressed in a specified tissue type (e.g., breast versus liver). The organism is selected from the group consisting of an animal, a vertebrate, an avian, a mammal, a human patient, a pet, a farm animal, a plant, and a plant root. In a further aspect, the target enzyme is present in the pathogen or in the infected cell but normally absent in the host or in uninfected host cells.

Although the method can utilize privately generated databases, it also can be practiced using publicly available databases, as exemplified below. Examples of databases include, but are not limited to commercially available genomic and protein databases (e.g., LifeSeq® available from Incyte Genomics, Inc.). Examples of public domain databases containing information that can be processed according to the invention can be accessed at a number of internet locations or Web sites. One such database is located at a Web site called WIT (a world wide web based system to support the curation of functional assignments made to genes, now “ERGO”) maintained by the Argonne National Laboratory of the University of Chicago. Another such database is located at a web site called KEGG (Kyoto Encyclopedia of Genes and Genomes) currently maintained by the Institute for Chemical Research at Kyoto University, Japan. The actual URL (universal resource locator) used to access WIT can change, but has recently been used as http://wit.mcs.anl.gov/WIT2. Similarly, the KEGG site http://www.blast.genome.ad.jp/kegg/kegg2.html can be used.

In one embodiment, the databases are searched for enzymes using their respective Enzyme Commission Numbers (“EC”). ECs uniquely identify individual enzymes and are interpretable in terms of the reaction mechanism of each enzyme so named. Thus, these numbers can be useful for sorting through large numbers of candidate enzyme entries in a variety of databases.

In another embodiment of the invention, the method requires selecting from a database an enzyme that is expressed by an infectious agent or in an infected cell and comparing these results with a database of expressed enzymes in at least one different class of organisms. In one aspect, these results are further compared to a database comprising enzymes expressed by yet a different class of organisms to identify an enzyme that is expressed in at least one class of organisms but not expressed in another class of organisms. For example, the method is useful to identify target enzymes present in a pathological organism but absent in an uninfected subject host such as enzymes present in pathogenic bacteria but not in human cells.

In a further embodiment of the present invention, a list of the identified enzymes may also be outputted. In another embodiment of the present invention, the identified enzymes may further be organized into a first set of enzymes capable of being placed into metabolic pathways and a second set of enzymes not capable of being placed into metabolic pathways. The first and second sets of enzymes may then be displayed such that the first set of enzymes is distinguishable from the second set of enzymes. In such an embodiment, a third data structure may be queried to organize the identified enzymes.

The methods described herein selected for iECTA targets shown in FIGS. 7A and 7B. However, new entries are added everyday to the databases. Accordingly, the practice of this invention subsequent to the filing of the present application will identify iECTA enzymes not listed in FIG. 7A and 7B. In some embodiments, the newly identified enzymes are presently identified by percentage homology to an enzyme shown in FIGS. 7A and B. Also termed “biologically equivalent iECTA enzymes” are characterized by possessing at least 75%, or at least 80%, or at least 90% or at least 95% amino acid sequence homology as determined using a sequence alignment program under default parameters correcting for ambiguities in the sequence data, changes in nucleotide sequence that do not alter the amino acid sequence because of degeneracy of the genetic code, conservative amino acid substitutions and corresponding changes in nucleotide sequence, and variations in the lengths of the aligned sequences due to splicing variants or small deletions or insertions between sequences that do not affect function.

In a separate embodiment, a “biological equivalent” intends a protein sequence identified by BLAST search using our the iECTA sequence as input and that results in “hits” having E values indicating that the probability that the “hit” is due to chances is less than 1 in 1000, or 1 in 100, or 1 in 10. This identifies any protein that is related at all, even if the sequence similarity by alignment is less than 10%. Catalytically equivalent enzymes have been identified by BLAST search in this way, even when the % similarity is on the order of a few percent. Human telomerase is a good example of this, because it was identified by BLAST search using a protein sequence obtained from the corresponding enzyme of the ciliate Euplotes.

In general, iECTA enzymes having one or more of the following characteristics: 1) enzyme is expressed only by the pathogen of interest or in the cell infected with the pathogen; 2) enzyme is expressed by the pathogen of interest but not by the host organism; 3) enzyme is part of a critical biochemical pathway for the pathogen or cell infected by the pathogen; or 4) enzyme is or is analogous to an enzyme present in a “favorable reaction type” in the pathogen or in a cell infected by the pathogen.

Other examples of pathogen-specific enzymes include drug resistance enzymes expressed by those organisms. Examples include resistance plasmid-encoded drug-modifying enzymes (e.g., chloramphenicol acetyl transferase and other plasmid- or chromosomally-encoded enzymes like beta-lactamases, Table 1, Part C). Intrinsic ECTA targets differ from resistance ECTA targets only in that the intrinsic enzymes (e.g., viral encoded protease) are present or expressed in naive or untreated pathogens. Resistance enzymes are typically only expressed or expressed at elevated levels as a result of challenge by therapeutic agents such as enzyme inhibitors.

As noted above, the method of this invention, identifies enzymes that occur in one class of organisms, but NOT in another class. The “class” can be defined by the user. It is likely that, contained in the output list of enzymes, some enzymes will be more amenable than others to development for iECTA. The described technique allows for an examination of the original output list for enzymes with unique mechanisms of action (analogous to the enzymes described in Table 1, below).

TABLE 1
Examples of Enzyme Targets for ECTA Technology

| | | | | |
|---|---|---|---|---|
| | Example | | | |
| | Disease/ | Example | Mechanisms | Referenced |
| Enzyme | Pathogen | Inhibitors | of Resistance | (Examples) |

Part A. Examples of Endogenous Overexpressed Enzymes in Cancer

| | | | | |
|---|---|---|---|---|
| Thymidylate | Cancer | Fluoropyrimi- | Overexpression | Lonn et al. |
| synthase | | dines, | Mutations | (1996) |
| (TS) | | Tomudex, | Salvage | Kobayashi et |
| | | Multitargeted | Pathways | al. (1995) |
| | | Antifolates | | Jackman et al. |
| | | (MTA) | | (1995) |
| Dihydrofolate | Cancer | Methotrexate | Overexpression | Banerjee et al. |
| reductase | | | | (1995) |
| (DHFR) | | | | Bertino et al. |
| | | | | (1996) |
| Ornithine | Cancer | α-Difluorome- | Overexpression | Das et al. |
| decarboxylase | | thylornithine | | (2000) |
| (ODC) | | (DFMO) | | |
| Cyclin- | Cancer | Flavopiridol | Unknown | Ruas and |
| dependent | | | | Peters (1998) |
| Kinases 4 and | | | | Sausville et al. |
| 6 (cdk 4, 6) | | | | (1999) |

Part B. Virally Encoded Enzymes

| | | | | |
|---|---|---|---|---|
| Viral Protease | HIV, | Indinavir, | Mutations | Venturi et al. |
| | HCV | ritonavir | | (2000) |
| | | | | Blight et al. |
| | | | | (1998) |
| Reverse | HIV, other | AZT, other | Mutations | Shirasaka et. |
| Transcriptase | retrovirus | nucleoside or | | al. (1995) |
| | | Nonnucleoside | | Venturi et al |
| | | analogs | | (2000) |
| | | | | Casado et al. |
| | | | | (2000) |
| RNA- | HCV and | Peptide-based | Unknown | Blight et al. |
| dependent | other | Alpha- | | (1998) |
| RNA- | Flaviviruses | diketones | | Han et al. |
| polymerase | | | | (2000) |
| Neuraminidase | Influenza | Derivatives of | Mutations | Staschke et al. |
| (NA) | | 2-deoxy-2,3- | | (1995) |
| | | dehydro-N- | | Varghese et al. |
| | | acetylneuraminic | | (1998) |
| | | acid | | |
| | | (Neu5Ac2en) | | |
| DNA | Hepatitis | Lamivudine | Mutations | Malik et al. |
| polymerase | B | | | (2000) |
| (DNAse) | | | | |

Part C. Pathogen-Specific Enzyme

| | | | | |
|---|---|---|---|---|
| Acetolactate | Bacterial and | Herbicides | Overexpression | Whitcomb. |
| Synthase | Fungal | e.g., | Mutations | (1999) |
| (AcLS) | Infections | sulfonylurea | | Harms et al. |
| | | | | (1992) |
| Ketol-Acid | Bacterial and | N-Hydroxy-N- | Not described | Aulabaugh |
| Reductoisomerase | Fungal | isopropyl- | | and Schloss |
| (KARI) | Infections | oxamate | | (1990) |
| Beta-lactamase | Drug- | Clavulanic | Overexpression | Bonomo |
| (BL) | Resistant | acid | Mutations | et al (1999) |
| | Bacterial | Sulbactam | | |
| | Infections | | | |
| Dihydrofolate | Drug- | Trimethoprim | Mutations | Amyes et al, |
| reductase | Resistant | | | (1992) |
| (DHFR) | Bacterial | | | |
| | Infections | | | |
| Chloramphenical | Drug- | N/A | Overexpression | Kleanthous |
| Acetyl | Resistant | | | et al (1985) |
| Transferase | Bacterial | | | Shaw et al |
| (CAT) | Infections | | | (1988) |
| | | | | Shaw et al |
| | | | | (1991) |
| Peptidoglycan | Drug- | Methicillin | Mutations | Berger- |
| Glycosyltrans- | Resistant | Vancomycin | | Bachi et al |
| ferase (aka | Bacterial | | | (1989) |
| Penicillin | Infections | | | Hanaki et |
| Binding Protein | | | | al, (1998) |
| (PBP)) | | | | |
| Van A Peptide | Drug | Vancomycin | Mutations | Armstrong |
| Ligase | Resistant | LY333328 | | and Cohen |
| Van H Pyruvate | Bacterial | | | (1999) |
| D-Lactic Acid | Infections | | | Lessard |
| Convertase | | | | et al, (1999) |
| Van HD | | | | Arthur et al. |
| dehydrogenase | | | | (1999) |
| Van YD | | | | Casadewall |
| DD- | | | | et al. (1999) |
| carboxypeptidase | | | | |
| D-alanine | Mycobacteria | D-cycloserine | Overexpression | Caceres et |
| racemase | | | | al. (1997) |
| Mycolate | Tuburculosis | Thiolarto- | Not Known | Yuan et al |
| maturation | Mycobacteria | mycin | | (1998) |
| enzymes | | | | |
| Catalase | Tuburculosis | Isoniazid | Mutation | Meisel et al |
| Peroxide | Mycobacteria | | | (1998) |
| Kat G-encoded | Mycobacteria | Isoniazid | Overexpression | Mdluli et al. |
| catalase | | | and mutation | (1998) |
| InhA, NADH- | Mycobacteria | Isoniazid | Overexpression | Miesel et al. |
| dependent enoyl | | | and mutation | (1998) |
| acyl carder | | | | |
| protein reductase | | | | |
| Pyrazine | Mycobacteria | Pyrazinamide | Mutation | Raynaud et |
| amidase | | | | al. (1999) |
| CMA-1, related | Mycobacteria | Unknown | Unknown | Yuan et al. |
| to E. coli | | | | (1995) |
| cyclopropane | | | | |
| fatty acid | | | | |
| synthase | | | | |

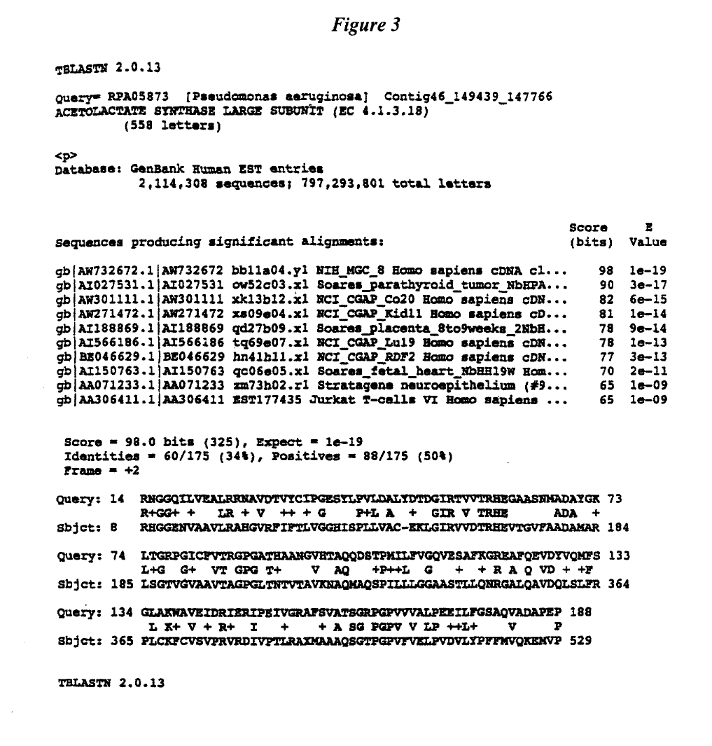

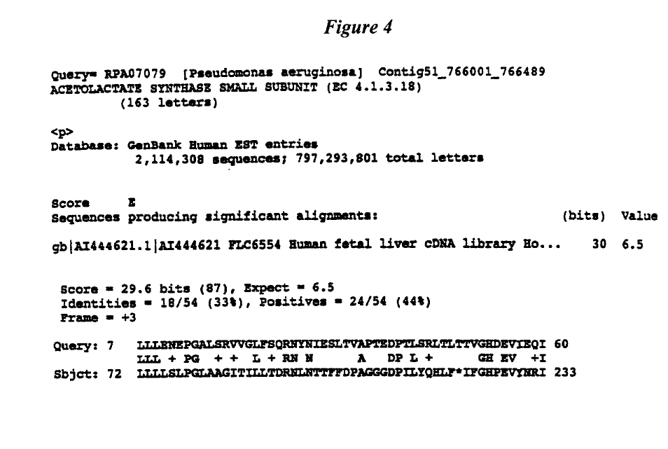

The methods can be practiced using local alignment search algorithms (i.e., BLAST, FASTA) or by directly searching various genome sequence databases, see for example, FIGS. 3 and 4. This method can be applied to any target organism for which DNA sequence information is available. These databases include microbial genome databases, human genome databases, and expressed sequence tag databases. This invention provides a way of querying databases using genome sequence information to identify potential iECTA enzymes. For example, an open reading frame (ORF) amino acid sequence is obtained for each target using a search program to determine which of these represents an enzyme (EC number) according to current annotation. Using a local alignment algorithm such as BLAST, the amino acid sequence of the candidate enzyme is compared with each sequence of a database consisting of human expressed sequence tags. The result obtained by these comparisons can be interpreted as a probability that an enzyme represented by sequence data is expressed in human cells. This would indicate that the target organism shared a common ancestor with humans and that the enzyme from humans and the target organism are related. If the enzymes are so related, they may share traits such as similar mechanism of action and similar substrate specificity and this might counter indicate the usefulness of related enzymes as iECTA targets.

As noted above, the methods of this invention identify enzymes and metabolic pathways present in the pathogenic organisms, but absent in the host, and as such, are a source of selectivity. For example, some pathways, as well as the enzymes involved, have only been found in bacteria, fungi and plants and not in mammalian cells. One example is the synthesis of “essential” amino acids—amino acids that animals cannot synthesize and must ingest with food (see Table 2 and Nelson and Cox (1972)).

TABLE 2
Amino Acid Biosynthetic Pathways Not Present in Humans

| | |
|---|---|
| Threonine | Leucine |
| Methionine | Histidine |
| Valine | Phenylalanine |
| Isoleucine | Tryptophan |
| Lysine | |

This invention also provides a means of uncovering potential enzyme targets in pathways that are common only in biochemical outcome but differ in route taken. For example, cysteine is not an essential amino acid, but many pathogenic microbes synthesize cysteine in a fashion different from humans and other higher organisms. The enzyme cysteine synthase (EC 4.2.99.8) is not found in humans, drosophila, or mus muscalus according to our search algorithm and is therefore a potential ECTA target.

These promising results suggest the utility of the iECTA approach in treating other diseases characterized by expression of pathogen specific enzymes (Table 1; Parts B and C). For example, HIV-1 protease (Table 1; Part B) is required for specific cleavage of virus-encoded gp160 to yield gp120, which is necessary for virus maturation (Markowitz and Ho (1996)). Protease inhibitors have been used for patient treatment, and inhibitor-resistant mutants of the enzyme have been described (Shirasaka, et al. (1995) and Venturi, et al. (2000)). A possible iECTA compound is based on the structure of a pharmacophore derived from the natural gp160 cleavage site (Kirkpatrick, et al. (1999); Bohocek and Martin (1997); and Ekins, et al. (1999)), such that reaction with protease leads to the formation of a toxin in virus infected cells. Because HIV protease is present only in virus-infected cells, only those cells will be affected following exposure to an HIV protease iECTA compound. Other examples of possible virally encoded iECTA targets include essential viral-specific replication enzymes like reverse transcriptase encoded by retroviruses (e.g., HIV), and RNA-dependent RNA polmerase encoded by flaviviruses (e.g., HCV). For iECTA applications it is critical that the target enzyme is required for viability or pathogenesis of the infectious agent. For this reason, dispensable viral enzymes are not preferred targets. An important example of a dispensable viral enzyme is the herpes virus-encoded thymidine kinase (Coen (1996) and Oram, et al. (2000)). For this reason, the herpes virus-encoded thymidine kinase is not included as a preferred target in Table 2, while essential enzymes like reverse transcriptase, RNA-dependent RNA polymerase and virally-encoded proteases are included. Pathogen specific enzymes are listed in Table 1 (Part C).

Hardware Implementation of the Methods and Systems

The methods of this invention operate on a typical computer system. The computer system can include various input devices such as a keyboard. The computer system also includes a processor such as CPU and internal memory. The processor may be a special purpose processor with database processing capabilities or it may be a general-purpose processor. The memory may comprise various types of memory, including RAM, ROM, and the like. The computer system also includes external storage that includes devices such as disks, CD ROMs, ASICs, external RAM, external ROM and the like.

The present invention can be implemented as part of the processor or as a program residing in memory and external storage and running on processor or as a combination of program and specialized hardware. When in memory and/or external storage the program can be in a RAM, a ROM, an internal or external disk, a CD ROM, an ASIC or the like. In general, when implemented as a program or in part as a program, the program can be encoded on any computer-readable medium or combination of computer-readable media, including but not limited to a RAM, a ROM, a disk, an ASIC, a PROM and the like. The computer system also includes a display and, optionally, an output device such as a printer.

The computer system can run any operating system and can be implemented in any computer programming language or combination of computer programming languages, although preferably it is implemented, at least in part, in a language which is suitable for database access and manipulation.

Thus, in another aspect, this invention provides a system for identifying enzymes for designing Enzyme Catalyzed Therapeutic Activation (ECTA) compounds, comprising logic for searching a first data structure to obtain a first set of information relating to one or more enzymes associated with a target organism that are expressed in a pathological cell or by a infectious agent or in an infected cell as compared to a suitable control and logic for searching one or more other data structures to obtain one or more additional sets of information relating to one or more expressed enzymes associated with one or more additional classes of organisms that are expressed respective class. The system also comprises logic for comparing the first set of information to the one or more additional sets of information to identify enzymes in the first set of information that are not present in the one or more additional sets of information, wherein the identified enzymes are capable of being used to design ECTA compounds. In one embodiment, the enzymes are overexpressed as compared to a suitable control. In yet a further aspect, the overexpressed enzyme is the result of prior treatment, e.g., antiobiotic or chemotherapy. In another aspect, the system further comprises logic for outputting a list of the identified enzymes. In yet a further aspect, the system comprises logic for organizing the identified enzymes into a first set of enzymes capable of being placed into metabolic pathways and a second set of enzymes not capable of being placed into metabolic pathways; and logic for displaying the first and second sets of enzymes such that the first set of enzymes are distinguishable from the second set of enzymes. In a further embodiment, a third data structure is queried to organize the identified enzymes.

The system can be part of a network that is utilized to search at least one of the first data structure and the second data structure. Examples of suitable networks include, but are not limited to, a network capable of communicating utilizing TCP/IP or IPX protocols.

In a further aspect, the information relating to the one or more enzymes of the organism includes information about Enzyme Commission (EC) numbers of the one or more enzymes. In yet a further aspect, the one or more additional sets of information relating to the one or more expressed enzymes associated with one or more classes of organisms includes information about Enzyme Commission (EC) numbers of the one or more expressed enzymes.

This invention further provides a computer program product for identifying enzymes for designing Enzyme Catalyzed Therapeutic Activation (ECTA) compounds, comprising computer code for searching a first data structure to obtain a first set of information relating to one or more enzymes associated with a pathological cell, by an infectious agent or in an infected cell and computer code for searching one or more other data structures to obtain one or more additional sets of information relating to one or more expressed enzymes associated with one or more additional classes of organisms that are express. The program product also contains computer code for comparing the first set of information to the one or more additional sets of information to identify enzymes in the first set of information that are not present in the one or more additional sets of information, wherein the identified enzymes are capable of being used to design ECTA compounds. Further additions include, but are not limited to computer code for outputting a list of the identified enzymes, computer code for organizing the identified enzymes into a first set of enzymes capable of being placed into metabolic pathways and a second set of enzymes not capable of being placed into metabolic pathways; and computer code for displaying the first and second sets of enzymes such that the first set of enzymes are distinguishable from the second set of enzymes. When a third data structure is queried or searched, a third code is supplied to query and optionally organize the information. Optionally, the information regarding enzyme expression can be organized according to ECC number. The system can work on a stand alone computer system or be a component of a network. In one aspect, the network is capable of communicating utilizing TCP/IP or IPX protocols.

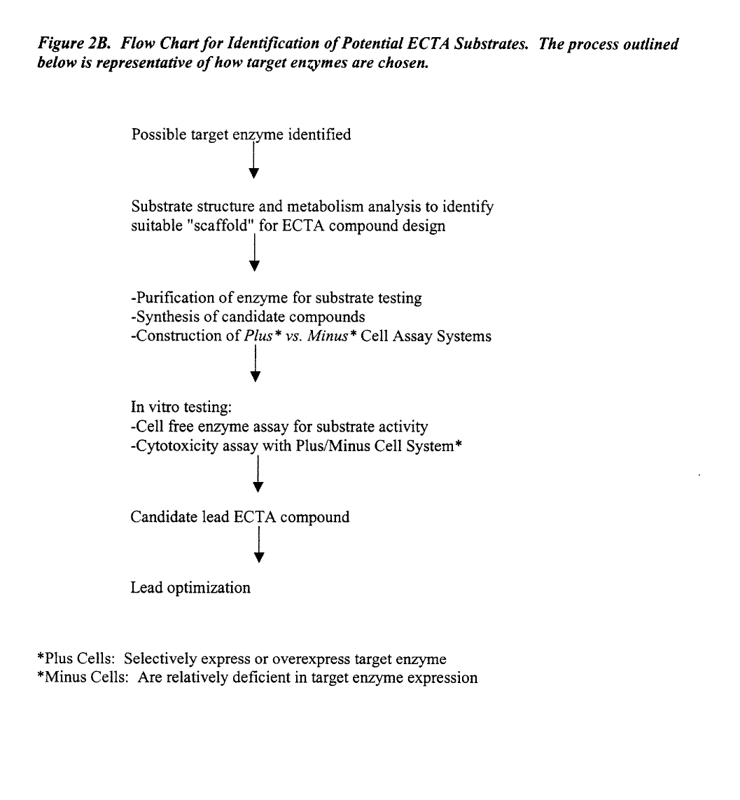

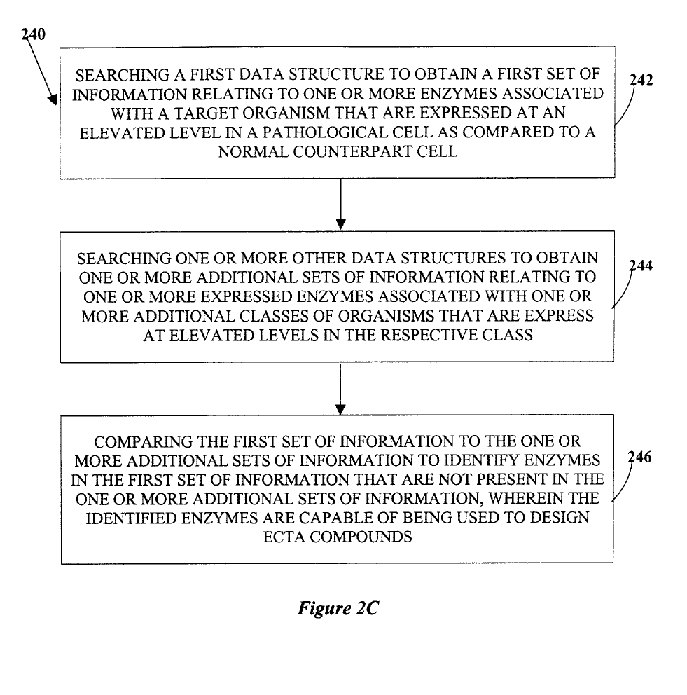

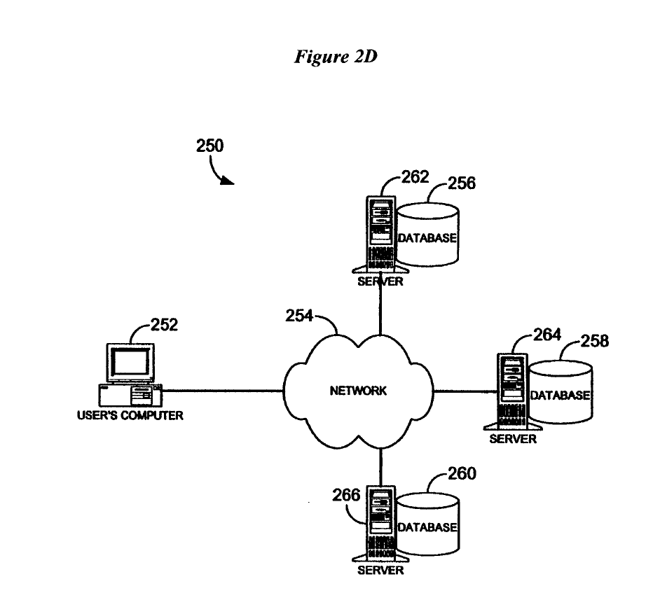

FIG. 2C is a flowchart for process 240 for identifying iECTA enzymes for designing iECTA compounds in accordance with an embodiment of the present invention. In operation 242, a first data structure is searched to obtain a first set of information relating to one or more enzymes associated with a target organism that are expressed at an elevated level in a pathological cell as compared to a normal counterpart cell or host cell. In operation 244, one or more other data structures are searched to obtain one or more additional sets of information relating to one or more expressed enzymes associated with one or more additional classes of organisms that are expressed at elevated levels in the respective class. The first set of information is compared to the one or more additional sets of information in operation 246 to identify enzymes in the first set of information that are not present in the one or more additional sets of information.

In one aspect of the present invention, a network may be utilized to search the first data structure and/or the second data structure. In such an aspect, the network may be capable of communicating utilizing TCP/IP and/or IPX protocols. In another aspect, the information relating to the one or more enzymes of the target organism may include information about Enzyme Commission (EC) numbers of the one or more enzymes. Similarly, the one or more additional sets of information relating to the one or more expressed enzymes associated with one or more classes of organisms may also include information about Enzyme Commission (EC) numbers of the one or more expressed enzymes. In a further aspect, the identified enzymes may be capable of being used to design iECTA compounds.

As an option, operations 244 and 246 may be executed sequentially for each additional database. In other words, operations 244 and 246 may be repeated for each additional database searched. For example, operations 244 and 246 may be executed for a first additional database (i.e., a second database) to obtain a first output which identifies enzymes in the first set of information that are not present in the information obtained from the first additional database. Operations 244 and 246 may then be executed utilizing the first output and a second additional database (i.e., a third database) to obtain a second output which identifies enzymes in the first set of information that are not present in the information obtained from the second additional database, and so on.

FIG. 2D is a schematic diagram of an illustrative system 250 capable of executing the process 240 for identifying enzymes for designing ECTA compounds set forth in FIG. 2C in accordance with an embodiment of the present invention. In particular, a user's computer 252 is connected via a network 254 (e.g., a LAN or a WAN such as the Internet) to a plurality of databases 256, 258, 260 (i.e., data structures). As illustrated in FIG. 2D, each database 256, 258, 260 may be hosted by a separate server 262, 264, 266 connected to the network. However, it should be understood that the databases may be hosted all on one server or on two servers, or even more than three servers.

One of the databases of the system 250 may contain information relating to one or more enzymes associated with a target organism that are expressed at an elevated level in a pathological cell as compared to a normal counterpart or host cell. A second of the databases may contain information relating to one or more expressed enzymes associated with another class of organisms that are express at elevated levels in the particular class. The user's computer 242 may be utilized to compare the information obtained from the databases.

FIG. 8 illustrates an exemplary system 1200 with a plurality of components 1202 in accordance with an embodiment of the present invention. As shown, such components include a network 1204 which take any form including, but not limited to a local area network, a wide area network such as the Internet, etc. Coupled to the network 1204 is a plurality of computers which may take the form of desktop computers 1206, laptop computers 1208, hand-held computers 1210, or any other type of computing hardware/software. As an option, the various computers may be connected to the network 1204 by way of a server 1212 which may be equipped with a firewall for security purposes. It should be noted that any other type of hardware or software may be included in the system and be considered a component thereof.

A representative hardware environment associated with the various components of FIG. 8 is depicted in FIG. 9. In the present description, the various sub-components of each of the components may also be considered components of the system. For example, particular software modules executed on any component of the system may also be considered components of the system. FIG. 9 illustrates a typical hardware configuration of a workstation in accordance with one embodiment having a central processing unit 1310, such as a microprocessor, and a number of other units interconnected via a system bus 1312.

The workstation shown in the figure includes a Random Access Memory (RAM) 1314, Read Only Memory (ROM) 1316, an I/O adapter 1318 for connecting peripheral devices such as disk storage units 1320 to the bus 1312, a user interface adapter 1322 for connecting a keyboard 1324, a mouse 1326, a speaker 1328, a microphone 1332, and/or other user interface devices such as a touch screen (not shown) to the bus 1312, communication adapter 1334 for connecting the workstation to a communication network 1335 (e.g., a data processing network) and a display adapter 1336 for connecting the bus 1312 to a display device 1338.

Software Implementation of the Methods and Systems

The workstation typically has resident thereon an operating system such as, for example: the Microsoft Windows NT or Windows 95/98/2000 Operating System (OS), the IBM OS/2 operating system, the MAC OS, or UNIX operating system. Those skilled in the art will appreciate that the present invention may also be implemented on platforms and operating systems other than those mentioned.

An embodiment may be written using JAVA. C. and the C++ language and utilizes object oriented programming methodology or any other means. Object oriented programming (OOP) has become increasingly used to develop complex applications. As OOP moves toward the mainstream of software design and development, various software solutions require adaptation to make use of the benefits of OOP. A need exists for these principles of OOP to be applied to a messaging interface of an electronic messaging system such that a set of OOP classes and objects for the messaging interface can be provided. OOP is a process of developing computer software using objects, including the steps of analyzing the problem, designing the system, and constructing the program. An object is a software package that contains both data and a collection of related structures and procedures. Since it contains both data and a collection of structures and procedures, it can be visualized as a self-sufficient component that does not require other additional structures, procedures or data to perform its specific task. OOP, therefore, views a computer program as a collection of largely autonomous components, called objects, each of which is responsible for a specific task. This concept of packaging data, structures, and procedures together in one component or module is called encapsulation.

In general, OOP components are reusable software modules which present an interface that conforms to an object model and which are accessed at run-time through a component integration architecture. A component integration architecture is a set of architecture mechanisms which allow software modules in different process spaces to utilize each others capabilities or finctions. This is generally done by assuming a common component object model on which to build the architecture. It is worthwhile to differentiate between an object and a class of objects at this point. An object is a single instance of the class of objects, which is often just called a class. A class of objects can be viewed as a blueprint, from which many objects can be formed.

OOP allows the programmer to create an object that is a part of another object. For example, the object representing a piston engine is to have a composition-relationship with the object representing a piston. In reality, a piston engine comprises a piston, valves and many other components; the fact that a piston is an element of a piston engine can be logically and semantically represented in OOP by two objects.

OOP also allows creation of an object that “depends from” another object. If there are two objects, one representing a piston engine and the other representing a piston engine wherein the piston is made of ceramic, then the relationship between the two objects is not that of composition. A ceramic piston engine does not make up a piston engine. Rather it is merely one kind of piston engine that has one more limitation than the piston engine; its piston is made of ceramic. In this case, the object representing the ceramic piston engine is called a derived object, and it inherits all of the aspects of the object representing the piston engine and adds further limitation or detail to it. The object representing the ceramic piston engine “depends from” the object representing the piston engine. The relationship between these objects is called inheritance.

When the object or class representing the ceramic piston engine inherits all of the aspects of the objects representing the piston engine, it inherits the thermal characteristics of a standard piston defined in the piston engine class. However, the ceramic piston engine object overrides these ceramic specific thermal characteristics, which are typically different from those associated with a metal piston. It skips over the original and uses new finctions related to ceramic pistons. Different kinds of piston engines have different characteristics, but may have the same underlying functions associated with it (e.g., how many pistons in the engine, ignition sequences, lubrication, etc.). To access each of these finctions in any piston engine object, a programmer would call the same functions with the same names, but each type of piston engine may have different/overriding implementations of functions behind the same name. This ability to hide different implementations of a function behind the same name is called polymorphism and it greatly simplifies communication among objects.

With the concepts of composition-relationship, encapsulation, inheritance and polymorphism, an object can represent just about anything in the real world. In fact, one's logical perception of the reality is the only limit on determining the kinds of things that can become objects in object-oriented software. Some typical categories are as follows:

Objects can represent physical objects, such as automobiles in a traffic-flow simulation, electrical components in a circuit-design program, countries in an economics model, or aircraft in an air-traffic-control system.

Objects can represent elements of the computer-user environment such as windows, menus or graphics objects.

An object can represent an inventory, such as a personnel file or a table of the latitudes and longitudes of cities.

An object can represent user-defined data types such as time, angles, and complex numbers, or points on the plane.

With this enormous capability of an object to represent just about any logically separable matters, OOP allows the software developer to design and implement a computer program that is a model of some aspects of reality, whether that reality is a physical entity, a process, a system, or a composition of matter. Since the object can represent anything, the software developer can create an object which can be used as a component in a larger software project in the future.

If 90% of a new OOP software program consists of proven, existing components made from preexisting reusable objects, then only the remaining 10% of the new software project has to be written and tested from scratch. Since 90% already came from an inventory of extensively tested reusable objects, the potential domain from which an error could originate is 10% of the program. As a result, OOP enables software developers to build objects out of other, previously built objects.

This process closely resembles complex machinery being built out of assemblies and sub-assemblies. OOP technology, therefore, makes software engineering more like hardware engineering in that software is built from existing components, which are available to the developer as objects. All this adds up to an improved quality of the software as well as an increased speed of its development.

Programming languages are beginning to fully support the OOP principles, such as encapsulation, inheritance, polymorphism, and composition-relationship. With the advent of the C++ language, many commercial software developers have embraced OOP. C++ is an OOP language that offers a fast, machine-executable code. Furthermore, C++ is suitable for both commercial-application and systems-programming projects. For now, C++ appears to be the most popular choice among many OOP programmers, but there is a host of other OOP languages, such as Smalltalk, Common Lisp Object System (CLOS), and Eiffel. Additionally, OOP capabilities are being added to more traditional popular computer programming languages such as Pascal.

The benefits of object classes can be summarized, as follows:

Objects and their corresponding classes break down complex programming problems into many smaller, simpler problems.

Encapsulation enforces data abstraction through the organization of data into small, independent objects that can communicate with each other. Encapsulation protects the data in an object from accidental damage, but allows other objects to interact with that data by calling the object's member flnctions and structures.

Subclassing and inheritance make it possible to extend and modify objects through deriving new kinds of objects from the standard classes available in the system. Thus, new capabilities are created without having to start from scratch.

Polymorphism and multiple inheritance make it possible for different programmers to mix and match characteristics of many different classes and create specialized objects that can still work with related objects in predictable ways.

Class hierarchies and containment hierarchies provide a flexible mechanism for modeling real-world objects and the relationships among them.

Libraries of reusable classes are useful in many situations, but they also have some limitations. For example:

Complexity. In a complex system, the class hierarchies for related classes can become extremely confusing, with many dozens or even hundreds of classes.

Flow of control. A program written with the aid of class libraries is still responsible for the flow of control (i.e., it must control the interactions among all the objects created from a particular library).

The programmer has to decide which functions to call at what times for which kinds of objects.

Duplication of effort. Although class libraries allow programmers to use and reuse many small pieces of code, each programmer puts those pieces together in a different way. Two different programmers can use the same set of class libraries to write two programs that do exactly the same thing but whose internal structure (i.e., design) may be quite different, depending on hundreds of small decisions each programmer makes along the way. Inevitably, similar pieces of code end up doing similar things in slightly different ways and do not work as well together as they should.

Class libraries are very flexible. As programs grow more complex, more programmers are forced to reinvent basic solutions to basic problems over and over again. A relatively new extension of the class library concept is to have a framework of class libraries. This framework is more complex and consists of significant collections of collaborating classes that capture both the small scale patterns and major mechanisms that implement the common requirements and design in a specific application domain. They were first developed to free application programmers from the chores involved in displaying menus, windows, dialog boxes, and other standard user interface elements for personal computers.

Frameworks also represent a change in the way programmers think about the interaction between the code they write and code written by others. In the early days of procedural programming, the programmer called libraries provided by the operating system to perform certain tasks, but basically the program executed down the page from start to finish, and the programmer was solely responsible for the flow of control. This was appropriate for printing out paychecks, calculating a mathematical table, or solving other problems with a program that executed in just one way.

The development of graphical user interfaces began to turn this procedural programming arrangement inside out. These interfaces allow the user, rather than program logic, to drive the program and decide when certain actions should be performed. Today, most personal computer software accomplishes this by means of an event loop which monitors the mouse, keyboard, and other sources of external events and calls the appropriate parts of the programmer's code according to actions that the user performs. The programmer no longer determines the order in which events occur. Instead, a program is divided into separate pieces that are called at unpredictable times and in an unpredictable order. By relinquishing control in this way to users, the developer creates a program that is much easier to use. Nevertheless, individual pieces of the program written by the developer still call libraries provided by the operating system to accomplish certain tasks, and the programmer must still determine the flow of control within each piece after it's called by the event loop. Application code still “sits on top of” the system.

Even event loop programs require programmers to write a lot of code that should not need to be written separately for every application. The concept of an application framework carries the event loop concept further. Instead of dealing with all the nuts and bolts of constructing basic menus, windows, and dialog boxes and then making these things all work together, programmers using application frameworks start with working application code and basic user interface elements in place. Subsequently, they build from there by replacing some of the generic capabilities of the framework with the specific capabilities of the intended application.

Application frameworks reduce the total amount of code that a programmer has to write from scratch. However, because the framework is really a generic application that displays windows, supports copy and paste, and so on, the programmer can also relinquish control to a greater degree than event loop programs permit. The framework code takes care of almost all event handling and flow of control, and the programmer's code is called only when the framework needs it (e.g., to create or manipulate a proprietary data structure).

A programmer writing a framework program not only relinquishes control to the user (as is also true for event loop programs), but also relinquishes the detailed flow of control within the program to the framework. This approach allows the creation of more complex systems that work together in interesting ways, as opposed to isolated programs, having custom code, being created over and over again for similar problems.

Thus, as is explained above, a framework basically is a collection of cooperating classes that make up a reusable design solution for a given problem domain. It typically includes objects that provide default behavior (e.g., for menus and windows), and programmers use it by inheriting some of that default behavior and overriding other behavior so that the framework calls application code at the appropriate times.

There are three main differences between frameworks and class libraries:

Behavior versus protocol. Class libraries are essentially collections of behaviors that you can call when you want those individual behaviors in your program. A framework, on the other hand, provides not only behavior but also the protocol or set of rules that govern the ways in which behaviors can be combined, including rules for what a programmer is supposed to provide versus what the framework provides.

Call versus override. With a class library, the programmer instantiates objects and calls their member functions. It is possible to instantiate and call objects in the same way with a framework (i.e., to treat the framework as a class library), but to take full advantage of a framework's reusable design, a programmer typically writes code that overrides and is called by the framework. The framework manages the flow of control among its objects. Writing a program involves dividing responsibilities among the various pieces of software that are called by the framework rather than specifying how the different pieces should work together.

Implementation versus design. With class libraries, programmers reuse only implementations, whereas with frameworks, they reuse design. A framework embodies the way a family of related programs or pieces of software work. It represents a generic design solution that can be adapted to a variety of specific problems in a given domain. For example, a single framework can embody the way a user interface works, even though two different user interfaces created with the same framework might solve quite different interface problems.

Thus, through the development of frameworks for solutions to various problems and programming tasks, significant reductions in the design and development effort for software can be achieved. A preferred embodiment of the invention utilizes HyperText Markup Language (HTML) to implement documents on the Internet together with a general-purpose secure communication protocol for a transport medium between the client and the Newco. HTTP or other protocols could be readily substituted for HTML without undue experimentation. Information on these products is available in T. Berners-Lee, D. Connoly, “RFC 1866: Hypertext Markup Language—2.0” (Nov. 1995); and R. Fielding, H, Frystyk, T. Berners-Lee, J. Gettys and J. C. Mogul, “Hypertext Transfer Protocol—HTTP/1.1: HTTP Working Group Internet Draft” (May 2, 1996). HTML is a simple data format used to create hypertext documents that are portable from one platform to another. HTML documents are SGML documents with generic semantics that are appropriate for representing information from a wide range of domains. HTML has been in use by the World-Wide Web global information initiative since 1990. HTML is an application of ISO Standard 8879; 1986 Information Processing Text and Office Systems; Standard Generalized Markup Language (SGML).

To date, Web development tools have been limited in their ability to create dynamic Web applications which span from client to server and interoperate with existing computing resources. Until recently, HTML has been the dominant technology used in development of Web-based solutions. However, HTML has proven to be inadequate in the following areas:

Poor performance;

Restricted user interface capabilities;

Can only produce static Web pages;

Lack of interoperability with existing applications and data; and

Inability to scale.

Sun Microsystems's Java language solves many of the client-side problems by:

Improving performance on the client side;

Enabling the creation of dynamic, real-time Web applications; and

Providing the ability to create a wide variety of user interface components.

With Java, developers can create robust User Interface (UI) components. Custom “widgets” (e.g., real-time stock tickers, animated icons, etc.) can be created, and client-side performance is improved. Unlike HTML, Java supports the notion of client-side validation, off loading appropriate processing onto the client for improved performance. Dynamic, real-time Web pages can be created. Using the above-mentioned custom UI components, dynamic Web pages can also be created.

Sun's Java language has emerged as an industry-recognized language for “programming the Internet.” Sun defines Java as: “a simple, object-oriented, distributed, interpreted, robust, secure, architecture-neutral, portable, high-performance, multithreaded, dynamic, buzzword-compliant, general-purpose programming language. Java supports programming for the Internet in the form of platform-independent Java applets.” Java applets are small, specialized applications that comply with Sun's Java Application Programming Interface (API) allowing developers to add “interactive content” to Web documents (e.g., simple animations, page adornments, basic games, etc.). Applets execute within a Java-compatible browser (e.g., Netscape Navigator) by copying code from the server to client. From a language standpoint, Java's core feature set is based on C++. Sun's Java literature states that Java is basically, “C++ with extensions from Objective C for more dynamic method resolution.”

Another technology that provides similar function to JAVA is provided by Microsoft and ActiveX Technologies, to give developers and Web designers wherewithal to build dynamic content for the Internet and personal computers. ActiveX includes tools for developing animation, 3-D virtual reality, video and other multimedia content. The tools use Internet standards, work on multiple platforms, and are being supported by over 100 companies. The group's building blocks are called ActiveX Controls, small, fast components that enable developers to embed parts of software in hypertext markup language (HTML) pages. ActiveX Controls work with a variety of programming languages including Microsoft Visual C++, Borland Delphi, Microsoft Visual Basic programming system and, in the future, Microsoft's development tool for Java, code named “Jakarta.” ActiveX Technologies also includes ActiveX Server Framework, allowing developers to create server applications. One of ordinary skill in the art readily recognizes that ActiveX could be substituted for JAVA without undue experimentation to practice the invention.

iECTA Targets for Bacterial and Fungal Infections

In this embodiment of this invention, the method requires comparing the results of a database search of enzymes expressed in an infected cell or by an infectious agent with a database search for enzymes expressed by a different class of organisms to identify an enzyme that is expressed in at least one class of organisms but not expressed in another class of organisms. In an alternative embodiment, additional organism can be searched. In a further embodiment, the enzyme is overexpressed in the first class of organism as compared to the second class of organism or vice versa.

By searching the WIT database (now ERGO) EC 3.5.2.7 was found to occur in the genomes of a number of pathogenic organisms, including Enterococcus faecalis, Helicobacterpylori, Pseudomonas aeruginosa, and Yersinia pestis. The amino acid sequence of the enzyme was also obtained from this database, and by using the tBLASTn algorithm to search a database with human gene sequences, it was found that the smallest sum probability 0.048 indicates that there is no human gene in the gene index that has a significant degree of similarity to the bacterial EC 3.5.2.7. This enzyme thus has no homolog in the human gene index, and is therefore a target ECTA enzyme.

Indeed, the practice of this method identified several hundred iECTA target enzymes from 51 pathogenic organisms (See FIG. 7A and 7B, which list all the enzymes associated with all the currently annotated pathogens). Natural and ECTA substrates were identified for related groups or sub-classes of enzymes with specific examples detailed in order to exemplify, but not limit, the invention.

Beta-lactamase is an enzyme expressed by bacteria and its expression renders them resistant to beta-lactam antibiotics (Schaechter et al., 1993). Applicant previously identified this enzyme as an ECTA enzyme based on its overexpression as result of prior antibiotic therapy, see PCT Application No. PCT/US98/27493. Thus, in one aspect, beta-lactamase and peptide deformylase are specifically excluded as an iECTA enzyme.

However, the method of the present invention also identified beta-lactamase as an iECTA target enzyme. Other examples of pathogen-specific, drug resistance enzymes include resistance plasmid-encoded drug-modifying enzymes (e.g. chloramphenicol acetyl transferase and other plasmid- or chromosomally-encoded enzymes (Table 1, Part C).

Table 1, Parts B and C provides examples of enzyme targets for ECTA technology which suggest the utility of the ECTA approach in treating other diseases characterized by expression of pathogen specific enzymes.

Additional pathogen specific enzymes are listed in Table 1 (Part C), e.g., the two first enzymes of the branched chain amino acids (BCAA) pathway (acetolactate synthase and ketol-acid reductoisomerase). These enzymes are only functional in bacteria, fungi, and plants, not in humans or most animals (Whitcomb 1999, Chipman et al., 1998). ECTA compounds targeting these enzymes will selectively attack infectious agents including bacteria and yeast with low toxicity to the host.

Selection of Favorable Reaction Type Using Enzyme Commission Number

In one aspect, the invention provides a method of selecting iECTA targets by identifying pathogen encoded enzymes that catalyze favorable reaction types. This was accomplished by first selecting a specific enzyme that has been shown to be effective at metabolizing an ECTA substrate and then using Enzyme Commission numbers to identify enzymes that catalyze a similar reaction in another microorganism. Comparison of the sequences of these pathogen encoded enzymes with a human gene index or by comparison of EC numbers with EC numbers (enzymes) found in humans or other higher organisms was then performed to select individual target enzymes that are not present in human cells.

The International Enzyme Commission has developed a classification scheme that assigns each enzyme a unique number that specifies which of approximately 4500 distinct reaction types is catalyzed by the enzyme. This method is based on dividing enzyme catalyzed reactions into six classes, then further subdividing each of these classes, and so on through four levels of classification.

It is desirable in identifying ECTA enzymes to search for specific reaction types or similar reaction types in annotated databases of microbial genomes, for example. The Enzyme Commission numbering system provides a way of automating these searches. For example, beta-lactamase (EC 3.5.2.6) catalyzes a reaction that has proven to be amenable to development of ECTA substrates. Using this as an example, similar enzymes can be identified in target organisms by selecting EC numbers with varying degrees of similarity to find enzymes catalyzing similar reactions, i.e., the hydrolysis of cyclic arnides.

Since the EC classification system organizes reaction types using four levels, from most general to most similar, enzymes that catalyze similar reactions can be identified by varying the EC number for the last EC class number (i.e., the fourth number listed). An example is shown below where a chemically similar but unique ECTA molecule can be created for an enzyme related to the known ECTA target beta-lactamase (EC 3.5.2.6)

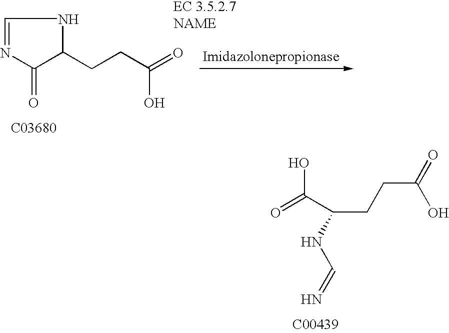

Design of iECTA Prodrugs

FIGS. 7A and 7B list iECTA target enzymes. The enzymes are organized according to EC number. Enzymes that share the first 3 numbers carry out chemical reactions in a very similar fashion, they just use different substrates. Substrate prodrugs have been designed based on the “natural” generic substrate.

The prodrugs were designed by evaluating the enzyme mechanism to determine chemically the best position to substitute the natural substrate with an ECTA prototoxophore. The prototoxophore is chosen based upon the enzyme active site and how the natural substrate binds this site. The prototoxophore can be a simple leaving group appended onto the natural substrate, but it does not necessarily resemble or mimic any or part of the natural substrate. The prototoxophore can be a reactive analogue of a natural fragmentation product that is released (unmasked) only after enzyme activation (for example see EC 4.1.3.27 anthranilate synthase). The prototoxophore can be a small chemical change to the natural substrate that takes advantage of the natural movement of electrons to create a highly reactive and toxic product that resembles the natural product. As used below, “unspecified” is intended to encompass all possible substituents, limited only by the laws of chemistry and physics and by what is tolerated by the ECTA enzyme target. The Enzyme Commission (EC) numbers define a specific enzyme reaction and therefore dictate the basic scaffold or substrate molecule to which substituents are added to create ECTA substrates or prodrugs. Although the toxin or toxoid is a specified substituent, Applicant intends that the toxin or toxoid be substituted at any appropriate atom on the compound, provided that the function of the compound is retained for its intended purpose.

EC 1 Oxidoreductases

All enzymes catalysing oxido-reductions belong to this class. The substrate oxidized is regarded as hydrogen or electron donor. The classification is based on ‘donor:acceptor oxidoreductase’. The recommended name is ‘dehydrogenase’, wherever this is possible; as an alternative, ‘acceptor reductase’ can be used. ‘Oxidase’ is used only where O2 is an acceptor. Classification is difficult in some cases, because of the lack of specificity towards the acceptor. A lack of specificity for the acceptor can be a major advantage when making unnatural (ECTA) substrates.

EC 1.1 Acting on the CH-OH group of donors

EC 1.1.1 With NAD or NADP as acceptor

EC 1.1.2 With a cytochrome as acceptor

EC 1.1.3 With oxygen as acceptor

EC 1.1.4 With a disulfide as acceptor

EC 1.1.5 With a quinone or similar compound as acceptor

EC 1.1.99 With other acceptors

EC 1.2 Acting on the aldehyde or oxo group of donors

EC 1.2.1 With NAD or NADP as acceptor

EC 1.2.2 With a cytochrome as acceptor

EC 1.2.3 With oxygen as acceptor

EC 1.2.4 With a disulfide as acceptor

EC 1.2.7 With an iron-sulfur protein as acceptor

EC 1.2.99 With other acceptors

EC 1.3 Acting on the CH-CH group of donors

EC 1.3.1 With NAD or NADP as acceptor

EC 1.3.2 With a cytochrome as acceptor

EC 1.3.3 With oxygen as acceptor

EC 1.3.5 With a quinone or related compound as acceptor

EC 1.3.7 With an iron-sulfur protein as acceptor

EC 1.3.99 With other acceptors

EC 1.4 Acting on the CH-NH2 group of donors

EC 1.4.1 With NAD or NADP as acceptor

EC 1.4.2 With a cytochrome as acceptor

EC 1.4.3 With oxygen as acceptor

EC 1.4.4 With a disulfide as acceptor

EC 1.4.7 With an iron-sulfur protein as acceptor

EC 1.4.99 With other acceptors

EC 1.5 Acting on the CH-NH group of donors

EC 1.5.1 With NAD or NADP as acceptor

EC 1.5.3 With oxygen as acceptor

EC 1.5.4 With a disulfide as acceptor

EC 1.5.5 With a quinone or similar compound as acceptor

EC 1.5.99 With other acceptors

EC 1.6 Acting on NADH or NADPH

EC 1.6.1 With NAD or NADP as acceptor

EC 1.6.2 With a heme protein as acceptor

EC 1.6.4 With a disulfide as acceptor

EC 1.6.5 With a quinone or similar compound as acceptor

EC 1.6.6 With a nitrogenous group as acceptor

EC 1.6.8 With a flavin as acceptor

EC 1.6.99 With other acceptors

EC 1.7 Acting on other nitrogenous compounds as donors

EC 1.7.2 With a cytochrome as acceptor

EC 1.7.3 With oxygen as acceptor

EC 1.7.7 With an iron-sulfur protein as acceptor

EC 1.7.99 With other acceptors

EC 1.8 Acting on a sulfur group of donors

EC 1.8.1 With NAD or NADP as acceptor

EC 1.8.2 With a cytochrome as acceptor

EC 1.8.3 With oxygen as acceptor

EC 1.8.4 With a disulfide as acceptor

EC 1.8.5 With a quinone as acceptor

EC 1.8.6 With nitrogenous group as acceptor

EC 1.8.7 With an iron-sulfur protein as acceptor

EC 1.8.99 With other acceptors

EC 1.9 Acting on a heme group of donors

EC 1.9.3 With oxygen as acceptor

EC 1.9.3.1 cytochrome-c oxidase

EC 1.9.3.2 Pseudomonas cytochrome oxidase

EC 1.10 Acting on diphenols and related substances as donors

EC 1.10.1 With NAD or NADP as acceptor

EC 1.10.2 With a cytochrome as acceptor

EC 1.10.3 With oxygen as acceptor

EC 1.10.99 With other acceptors

EC 1.11 Acting on a peroxide as acceptor

EC 1.11.1 Peroxidases

EC 1.11.1.1 NADH2 peroxidase

EC 1.11.1.2 NADPH2 peroxidase

EC 1.11.1.3 fatty-acid peroxidase

EC 1.11.1.4 now EC 1.13.11.11

EC 1.11.1.5 cytochrome-c peroxidase

EC 1.11.1.6 catalase

EC 1.11.1.7 peroxidase

EC 1.11.1.8 iodide peroxidase

EC 1.11.1.9 glutathione peroxidase

EC 1.11.1.10 chloride peroxidase

EC 1.11.1.11 L-ascorbate peroxidase

EC 1.11.1.12 phospholipid-hydroperoxide glutathione peroxidase

EC 1.11.1.13 manganese peroxidase

EC 1.11.1.14 diarylpropane peroxidase

EC 1.12 Acting on hydrogen as donor

EC 1.12.1 With NAD or NADP as acceptor

EC 1.12.2 With a cytochrome as acceptor

EC 1.12.7 With a iron-sulfur protein as acceptor

EC 1.12.99 With other acceptors

EC 1.13 Acting on single donors with incorporation of molecular oxygen (oxygenases)

EC 1.13.11 With incorporation of two atoms of oxygen

EC 1.13.12 With incorporation of one atom of oxygen (internal monooxygenases or internal mixed function oxidases)

EC 1.13.99 Miscellaneous

EC 1.14 Acting on paired donors, with incorporation or reduction of molecular oxygen

EC 1.14.11 With 2-oxoglutarate as one donor, and incorporation of one atom each of oxygen into both donors

EC 1.14.12 With NADH2 or NADPH2 as one donor, and incorporation of two atoms of oxygen into one donor

EC 1.14.13 With NAD or NADH as one donor, and incorporation of one atom of oxygen

EC 1.14.14 With reduced flavin or flavoprotein as one donor, and incorporation of one atom of oxygen

EC 1.14.15 With reduced iron-sulfur protein as one donor, and incorporation of one atom of oxygen

EC 1.14.16 With reduced pteridine as one donor, and incorporation of one atom of oxygen

EC 1.14.17 With reduced ascorbate as one donor, and incorporation of one atom of oxygen

EC 1.14.18 With another compound as one donor, and incorporation of one atom of oxygen

EC 1.14.99 Miscellaneous

EC 1.15 Acting on superoxide radicals as acceptor

EC 1.15.1

EC 1.15.1.1 Recommended name: superoxide dismutase

EC 1.16 Oxidising metal ions

EC 1.16.1 With NAD or NADP as acceptor

EC 1.16.3 With oxygen as acceptor

EC 1.17 Acting on CH2 groups

EC 1.17.1 With NAD or NADP as acceptor

EC 1.17.3 With oxygen as acceptor

EC 1.17.4 With disulfide as acceptor

EC 1.17.99 With other acceptors

EC 1.18 Acting on reduced ferredoxin as donor

EC 1.18.1 With NAD or NADP as acceptor

EC 1.18.6 With dinitrogen as acceptor

EC 1.18.99 With H+ as acceptor

EC 1.19 Acting on reduced flavodoxin as donor

EC 1.19.6 With dinitrogen as acceptor

EC 1.19.6.1 Recommended name: nitrogenase (flavodoxin)

EC 1.97 Other oxidoreductases

EC 1.97.1.1 chlorate reductase

EC 1.97.1.2 pyrogallol hydroxyltransferase

EC 1.97.1.3 sulfurreductase

EC 1.97.1.4 formate acetyltransferase activating enzyme

EC 1.97.1.5 arsenate reductase (glutaredoxin)

EC 1.97.1.6 arsenate reductase (donor)

EC 1.97.1.7 methylarsonate reductase

EC 2: Transferases—All enzymes that catalyzes a process involving reactions in which groups are transferred belong to this group.

EC 2.1 Transferring one-carbon groups

EC 2.1.1 methyltransferases

EC 2.1.2 hydroxymethyl-, fornyl- and related transferases

EC 2.1.3 carboxyl- and carbamoyltransferases

EC 2.1.4 amidinotransferases

EC 2.2 Transferring aldehyde or ketonic groups

EC 2.2.1 transketolases and transaldolases

EC 2.2.1.1 transketolase

EC 2.2.1.2 transaldolase

EC 2.2.1.3 formaldehyde transketolase

EC 2.2.1.4 acetoin-ribose-5-phosphate transaldolase

EC 2.3 Acyltransferases

EC 2.3.1 Transferring groups other than amino-acyl groups

EC 2.3.2 Aminoacyltransferases

EC 2.4 Glycosyltransferases

EC 2.4.1 Hexosyltransferases

EC 2.4.2 Pentosyltransferases

EC 2.4.99 Transferring Other Glycosyl Groups

EC 2.5 Transferring alkyl or aryl groups, other than methyl groups

EC 2.5.1 mixed examples

EC 2.6 Transferring nitrogenous groups

EC 2.6.1 Transaminases

EC 2.6.2 Amidinotransferases

EC 2.6.3 Oximinotransferases

EC 2.6.99 Transferring Other Nitrogenous Groups

EC 2.7 Transferring phosphorus-containing groups

EC 2.7.1 Phosphotransferases with an Alcohol Group as Acceptor

EC 2.7.2 Phosphotransferases with a carboxyl group as acceptor

EC 2.7.3 Phosphotransferases with a nitrogenous group as acceptor

EC 2.7.4 Phosphotransferases with a phosphate group as acceptor

EC 2.7.5 Phosphotransferases with regeneration of donors, apparently catalysing intramolecular transfers

EC 2.7.6 Diphosphotransferases

EC 2.7.7 Nucleotidyltransferases

EC 2.7.8 Transferases for other substituted phosphate groups

EC 2.7.9 Phosphotransferases with paired acceptors

EC 2.8 Transferring sulfur-containing groups

EC 2.8.1 Sulfurtransferases

EC 2.8.2 Sulfotransferases

EC 2.8.3 CoA-transferases

EC 2.9 Transferring selenium-containing groups

EC 2.9.1 Selenotransferases

EC 2.9.1.1 Recommended name: L-seryl-tRNASec selenium transferase

EC 3 Hydrolases This group includes any enzyme that catalyzes a process involving cleaving chemical groups with a molecule of water (excluding peptidases, see EC 3.4).

EC 3.1 Acting on ester bonds

EC 3.1.1 Carboxylic Ester Hydrolases

EC 3.1.2 Thiolester Hydrolases

EC 3.1.3 Phosphoric Monoester Hydrolases

EC 3.1.4 Phosphoric Diester Hydrolases

EC 3.1.5 Triphosphoric Monoester Hydrolases

EC 3.1.6 Sulfuric Ester Hydrolases

EC 3.1.7 Diphosphoric Monoester Hiydrolases

EC 3.1.8 Phosphoric Triester Hydrolases

EC 3.1.11 Exodeoxyribonucleases Producing 5′-Phosphomonoesters

EC 3.1.13 Exoribonucleases Producing 5′-Phosphomonoesters

EC 3.1.14 Exoribonucleases Producing 3′-Phosphomonoesters

EC 3.1.15 Exonucleases Active with either Ribo- or Deoxyribonucleic Acids and Producing 5′-Phosphomonoesters

EC 3.1.16 Exonucleases Active with either Ribo- or Deoxyribonucleic Acids and Producing 3′-Phosphomonoesters

EC 3.1.21 Endodeoxyribonucleases Producing 5′-Phosphomonoesters

EC 3.1.22 Endodeoxyribonucleases Producing other than 5′-Phosphomonoesters

EC 3.1.25 Site-Specific Endodeoxyribonucleases Specific for Altered Bases

EC 3.1.26 Endoribonucleases Producing 5′-Phosphomonoesters

EC 3.1.27 Endoribonucleases Producing other than 5′-Phosphomonoesters

EC 3.1.30 Endoribonucleases Active with either Ribo- or Deoxyribonucleic Acids and Producing 5′-Phosphomonoesters

EC 3.1.31 Endoribonucleases Active with either Ribo- or Deoxyribonucleic Acids and Producing 3′-Phosphomonoesters

EC 3.2 Glycosylases

EC 3.2.1 Glycosidases, i.e. enzymes hydrolysing 0- and S-glycosyl compounds

EC 3.2.2 Hydrolysing N-Glycosyl Compounds

EC 3.2.3 Hydrolysing S-Glycosyl Compounds (discontinued)

EC 3.3 Acting on ether bonds

EC 3.3.1 Trialkylsulfonium Hydrolases

EC 3.3.2 Ether Hydrolases

EC 3.4 Acting on peptide bonds (peptidases)

3.4.11 Aminopeptidases

3.4.13 Dipeptidases

3.4.14 Dipeptidyl-peptidases and tripeptidyl-peptidases

3.4.15 Peptidyl-dipeptidases

3.4.16 Serine-type carboxypeptidases

3.4.17 Metallocarboxypeptidases

3.4.18 Cysteine-type carboxypeptidases

3.4.19 Omega peptidases

3.4.21 Serine endopeptidases

3.4.22 Cysteine endopeptidases

3.4.23 Aspartic endopeptidases

3.4.24 Metalloendopeptidases

3.4.25 Threonine endopeptidases

3.4.99 Endopeptidases of unknown catalytic mechanism

EC 3.5 Acting on carbon-nitrogen bonds, other than peptide bonds

EC 3.5.1 In Linear Amides

EC 3.5.2 In Cyclic Amides

EC 3.5.3 In Linear Amidines

EC 3.5.4 In Cyclic Amidines

EC 3.5.5 In Nitriles

EC 3.5.99 In Other Compounds

EC 3.6 Acting on acid anhydrides

EC 3.6.1 In Phosphorus-Containing Anhydrides

EC 3.6.2 In Sulfonyl-Containing Anhydrides

EC 3.6.3 Acting on acid anhydrides; catalysing transmembrane movement of substances

EC 3.6.4 Acting on acid anhydrides; involved in cellular and subcellular movement.

EC 3.7 Acting on carbon-carbon bonds

EC 3.7.1 In Ketonic Substances

EC 3.7.1.1 oxaloacetase

EC 3.7.1.2 fumarylacetoacetase

EC 3.7.1.3 kynureninase

EC 3.7.1.4 phloretin hydrolase

EC 3.7.1.5 acylpyruvate hydrolase

EC 3.7.1.6 acetylpyruvate hydrolase

EC 3.7.1.7 b-diketone hydrolase

EC 3.7.1.8 2,6-dioxo-6-phenylhexa-3-enoate hydrolase

EC 3.7.1.9 2-hydroxymuconate-semialdehyde hydrolase

EC 3.7.1.10 cyclohexane-1,3-dione hydrolase

EC 3.8 Acting on halide bonds

EC 3.8.1 In C-Halide Compounds

EC 3.9 Acting on phosphorus-nitrogen bonds

EC 3.9.1.1 Recommended name: phosphoamidase

EC 3.10 Acting on sulfur-nitrogen bonds

EC 3.10.1.1 N-sulfoglucosamine sulfohydrolase

EC 3.10.1.2 cyclamate sulfohydrolase

EC 3.11 Acting on carbon-phosphorus bonds

EC 3.11.1.1 phosphonoacetaldehyde hydrolase

EC 3.11.1.2 phosphonoacetate hydrolase

EC 3.12 Acting on sulfur-sulfur bonds

EC 3.12.1.1 Recommended name: trithionate hydrolase

EC 4 Lyases

Lyases are enzymes cleaving C—C, C—O, C—N and other bonds by means other than by hydrolysis or oxidation. They differ from other enzymes in that two substrates are involved in one reaction direction, but only one in the other direction. When acting on the single substrate, a molecule is eliminated and this generates either a new double bond or a new ring. The systematic name is formed according to ‘substrate group-lyase’. In recommended names, expressions like decarboxylase, aldolase, etc. are used. ‘Dehydratase’ is used for those enzymes eliminating water. In cases where the reverse reaction is the more important, or the only one to be demonstrated, ‘synthase’ may be used in the name.

EC 4.1 Carbon-carbon lyases

EC 4.1.1 Carboxy-Lyases

EC 4.1.2 Aldehyde-Lyases

EC 4.1.3 Oxo-Acid-Lyases

EC 4.1.99 Other Carbon-Carbon Lyases

EC 4.2 Carbon-oxygen lyases

EC 4.2.1 Hydro-Lyases

EC 4.2.2 Acting on Polysaccharides

EC 4.2.99 Other Carbon-Oxygen Lyases

EC 4.3 Carbon-nitrogen lyases

EC 4.3.1 Ammonia-Lyases

EC 4.3.2 Amidine-Lyases

EC 4.3.3 Amine-Lyases

EC 4.3.99 Other Carbon-Nitrogen Lyases

EC 4.4 Carbon-sulfur lyases

EC 4.4 Carbon-Sulfur Lyases

EC 4.5 Carbon-Halide Lyases

EC 4.6 Phosphorus-Oxygen Lyases

EC 4.99 Other Lyases

EC 4.5 Carbon-halide lyases

EC 4.5.1.1 DDT-dehydrochlorinase

EC 4.5.1.2 3-chloro-D-alanine dehydrochlorinase

EC 4.5.1.3 dichloromethane dehalogenase

EC 4.5.1.4 L-2-amino-4-chloropent-4-enoate dehydrochlorinase

EC 4.5.1.5 S-carboxymethylcysteine synthase

EC 4.6 Phosphorus-oxygen lyases

EC 4.6.1.1 adenylate cyclase

EC 4.6.1.2 guanylate cyclase

EC 4.6.1.6 cytidylate cyclase

EC 4.99 Other lyases

EC 4.99.1.1 ferrochelatase

EC 4.99.1.2 alkylmercury lyase

EC 5 Isomerases

EC 5.1 Racemases and epimerases

EC 5.1.1 Acting on Amino Acids and Derivatives

EC 5.1.2 Acting on Hydroxy Acids and Derivatives

EC 5.1.3 Acting on Carbohydrates and Derivatives

EC 5.1.99 Acting on Other Compounds

EC 5.2 cis-trans-Isomerases

EC 5.2.1.1 maleate isomerase

EC 5.2.1.2 maleylacetoacetate isomerase

EC 5.2.1.3 retinal isomerase

EC 5.2.1.4 maleylpyruvate isomerase

EC 5.2.1.5 linoleate isomerase

EC 5.2.1.6 furylfuramide isomerase

EC 5.2.1.7 retinol isomerase

EC 5.2.1.8 peptidylprolyl isomerase

EC 5.2.1.9 famesol 2-isomerase

EC 5.2.1.10 2-chloro-4-carboxymethylenebut-2-en-1,4-olide isomerase

EC 5.2.1.11 4-hydroxyphenylacetaldehyde-oxime isomerase

EC 5.3 Intramolecular isomerases

EC 5.3.1 Interconverting Aldoses and Ketoses

EC 5.3.2 Interconverting Keto- and Enol-Groups

EC 5.3.3 Transposing C═C Bonds

EC 5.3.4 Transposing S—S Bonds

EC 5.3.99 Other Intramolecular Oxidoreductases

EC 5.4 Intramolecular transferases (mutases)

EC 5.4.1 Transferring Acyl Groups

EC 5.4.2 Phosphotransferases (Phosphomutases)

EC 5.4.3 Transferring Amino Groups

EC 5.4.99 Transferring Other Groups

EC 5.5 Intramolecular lyases

EC 5.5.1.1 muconate cycloisomerase

EC 5.5.1.2 3-carboxy-cis,cis-muconate cycloisomerase

EC 5.5.1.3 tetrahydroxypteridine cycloisomerase

EC 5.5.1.4 inositol-phosphate synthase

EC 5.5.1.5 carboxy-cis,cis-muconate cyclase

EC 5.5.1.6 chalcone isomerase

EC 5.5.1.7 chloromuconate cycloisomerase

EC 5.5.1.8 geranyl-diphosphate cyclase

EC 5.5.1.9 cycloeucalenol cycloisomerase

EC 5.5.1.10 a-pinene-oxide decyclase

EC 5.5.1.11 dichloromuconate cycloisomerase

EC 5.99 Other isomerases

EC 5.99.1.1 thiocyanate isomerase

EC 5.99.1.2 DNA topoisomerase

EC 5.99.1.3 DNA topoisomerase (ATP-hydrolysing)

EC 6 Ligases

EC 6.1 Forming carbon-oxygen bonds

EC 6.1.1 Ligases Forming Aminoacyl-tRNA and Related Compounds

EC 6.2 Forming carbon-sulfur bonds

EC 6.2.1 Acid-Thiol Ligases

EC 6.3 Forming carbon-nitrogen bonds

EC 6.3.1 Acid-Ammonia (or Amide) Ligases (Amide Synthases)

EC 6.3.2 Acid-D-Amino-Acid Ligases (Peptide Synthases)

EC 6.3.3 Cyclo-Ligases

EC 6.3.4 Other Carbon-Nitrogen Ligases

EC 6.3.5 Carbon-Nitrogen Ligases with Glutamine as Amido-N-Donor

EC 6.4 Forming carbon-carbon bonds

EC 6.4.1. 1 pyruvate carboxylase

EC 6.4.1.2 acetyl-CoA carboxylase

EC 6.4.1.3 propionyl-CoA carboxylase

EC 6.4.1.4 methylcrotonoyl-CoA carboxylase

EC 6.4.1.5 geranoyl-CoA carboxylase

EC 6.5 Forming phosphoric ester bonds

EC 6.5.1.1 DNA ligase (ATP)

EC 6.5.1.2 DNA ligase (NAD)

EC 6.5.1.3 RNA ligase (ATP)

EC 6.5.1.4 RNA-3′-phosphate cyclase

Biological Confirmation—Enzyme Assays

Also provided by this invention is a cell-free assay to confirm the efficacy of iECTA prodrugs by contacting the prodrug and enzyme in a cell-free system under conditions that favor activation of the prodrug by the enzyme. The enzymes and methods for expression of enzyme nucleic acids are known in the art, and therefore need not be reproduced herein. For example, all enzyme sequence information and reaction conditions are available online at one or more of the following sites: www./Brenda.bc.uni-koeln.de/ and www.expasy.ch/enzyme. As an example only, coding sequences for bacterial or fungal AcLS and KARI are cloned as described (Pang and Duggleby (1999); Poulsen and Stougaard (1989); and Hill et al. (1997)) and expressed in E. coli using an appropriate promoter system (Sambrook, et al. supra). Enzyme is purified using the “His-tag” system (Stratagene, La Jolla, Calif.). Enzyme assay for AcLS is done by methods described by Epelbaum, et al. (1998) and others (e.g., Hill, et al. (1997)). Cloning, expression and purification of KARI is as described by Hill and Duggleby (1999). Assays for KARI will be done similarly to those described by Epelbaum, et al. (1996) and Hill and Duggleby (1999).

This invention provides a method for confirming therapeutic potential for the treatment of infectious disease. The agent is considered a potential therapeutic agent if proliferation and/or replication of the infectious agent or the host cell are reduced relative to the cells in a control sample. Most preferably, the infectious agent is killed by the agent. Infected cells can be procaryotic (bacterial such as E. coli) or eucaryotic. The cells can be mammalian or non-mammalian cells, e.g., yeast cells, murine cells, rat cells, avian cells, human cells.

This invention also provides a quick and simple screening assay that will enable initial identification of compounds with at least some of the desired characteristics. In one aspect, the assay requires two cell types, the first being a control cell in which the target enzyme is not expressed or does not contain the infectious agent, or is expressed at a low level. The second cell type is the test cell, in which the target enzyme is expressed at a detectable level, e.g., a high level or a sample that contains the infectious agent. In a separate embodiment, a counterpart genetically modified to differentially express the target enzyme, or enzymes (containing the appropriate species of target enzyme) is used. More than one species of enzyme can be used to separately transfect separate host cells, so that the effect of the candidate drug on a target enzyme can be simultaneously compared to its effect on another enzyme or a corresponding enzyme from another species.

In another embodiment, a third target cell is used as a control because it receives an effective amount of a compound, such as, for example, the compounds shown below, which have been shown to be potent prodrugs. This embodiment is particularly useful to screen for new agents that are activated by iECTA enzymes.

In Vivo Testing for Preclinical Efficacy of iECTA Prodrugs

The in vitro assays are confirmed in animal or plant models infected with a pathogen expressing the target enzyme to determine in vivo efficacy. In vivo practice of the invention in an animal such as a rat or mouse provides a convenient animal model system that can be used prior to clinical testing of the therapeutic agent or prodrug. In this system, a potential prodrug will be successful if microbial load is reduced or the symptoms of the infection are ameliorated, each as compared to an untreated, infected animal. It also can be useful to have a separate negative control group of cells or animals which has not been infected, which provides a basis for comparison.

When practiced in vivo, the candidate prodrug is administered or delivered to the animal in effective amounts. As used herein, the term “administering” for in vivo and ex vivo purposes means providing the subject with an effective amount of the candidate prodrug effective to reduce microbial load. In these instances, the agent or prodrug may be administered with a pharmaceutically acceptable carrier. The agents, prodrugs and compositions of the present invention can be used in the manufacture of medicaments and for the treatment of humans and other animals by administration in accordance with conventional procedures, such as an active ingredient in pharmaceutical compositions.

Another aspect of this invention is a method for treating a subject or alleviating the symptoms of an infection by a pathogen in a subject, wherein the pathogen or a pathogen infected cell expresses an iECTA enzyme by delivering to the subject an effective amount of an iECTA prodrug compound that is converted to a toxin by the iECTA enzyme. Further provided is a method of treating a disease associated with an infection with a pathogen expressing an iECTA enzyme, or an infected host cell expressing an iECTA enzyme, by delivering to the subject an effective amount of an iECTA prodrug compound that is converted to a toxin by the iECTA enzyme. Examples of iECTA expressing pathogens and the corresponding diseases and symptoms caused by infection by these microorganisms, are provided in

Table 3, below. Yet further provided is a method for producing a medicament to treat a subject as indicated above, comprising combining an effective amount of a suitable iECTA prodrug and a pharmaceutically acceptable carrier.

TABLE 3

| iECTA | Disease or Symptom |
|---|---|
| Expressing Microorganism | Caused by Infection |
| Gram-Positive | |
| Staphylococcus aureus | major human pathogen, bacteremia, |
| | pneumonia |
| Staphylococcus epidermidis | urinary tract infections, osteomyelitis, |
| and other coagulase-negative | bacteremia |
| staphylococci | |
| Streptococcus pyogenes | bacteremia, lymphagitis, pneumonia |
| Streptococcus pneumoniae | pneumonia, otitis media, sinusitis |
| Streptococcus agalactiae | primary bacteremia, pneumonia, |
| | endocarditis, osteomyelitis |
| Enterococcus species | urinary tract infections, bacteremia, |
| | endocarditis, intra-abdominal and pelvic |
| | infections, neonatal sepsis |
| Gram-Negative | |
| Neisseria gonorrhoeae | genital infection, perihepatitis |
| Moraxella catarrhalis | otitis media, lower respiratory tract |
| | infections, pneumonia, bacteremia |
| Campylobacter jejuni | acute enteritis, acute colitis, |
| | bacteremia |
| Enterobacteriaceae (including | enteric infections, urinary tract |
| Escherichia, Salmonella, | infections, respiratory infections, |
| Klebsiella, Enterobacter) | bacteremia |
| Pseudomonas aeruginosa | endocarditis, respiratory infections, |
| | bacteremia, central nervous system |
| | infections |
| Acinetobacter species | respiratory tract infections, |
| | bacteremia, genitourinary |
| Haemophilus influenzae | meningitis, epiglottitis, pneumonia, |
| | bacteremia |

This invention also provides a method for treating or protecting plants from infection by applying an effective amount of the iECTA prodrug compound to the foliage, roots or the soil surrounding the plants or roots. These isolated compounds can be combined with known pesticides or insecticides.

Compounds within the present invention when used to treat or protect plants from infections, they can be formulated as wettable powders, granules and the like, or can be microencapsulated in a suitable medium and the like. Examples of other formulations include, but are not limited to soluble powders, wettable granules, dry flowables, aqueous flowables, wettable dispersible granules, emulsifiable concentrates and aqueous suspensions. Other suitable formulations will be known to those skilled in the art.

This invention further provides a method for administering the prodrug compound to fish in an amount effective to either prevent or treat an infection. The compound may be administered by incorporating the compound into the food supply for the fish. Alternatively, the compound may be added to the water in which fish live, or are contained within. Finally, the compound may be administered to the fish as a suitable pharmaceutical preparation. Other suitable formulations will be known to those skilled in the art.

When the iECTA prodrug compound is delivered to a subject such as a mouse, a rat or a human patient, the agent can be added to a pharmaceutically acceptable carrier and systemically or topically administered to the subject.

Animal models that can be used to test utility of candidate iECTA compounds set forth below have been described in the literature. Examples include animal models of infection by Staphyloccus aureus (Josefsson and Tartowski (1999) and Totsuka, et al. (1999)), Pneumocystis carinii (Tamburrini, et al. (1999)), enterococci (Zimbelman, et al. (1999)), multimicrobial peritonitis (Montravers, et al. (1999)), and fungal infections (Louie, et al. (1999)). In each case the candidate iECTA compound is compared with an antibiotic currently used to treat the disease. These experiments also provide the first test of therapeutic index. No toxicity of the candidate iECTA compound should be seen at doses necessary for eradication or control of disease. Preferably, doses that cause toxicity will be at least ten-fold higher than the doses needed for control or cure of the disease.

Administration in vivo can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents can be found below.

The agents and compositions of the present invention can be used in the manufacture of medicaments and for the treatment of humans and other animals by administration in accordance with conventional procedures, such as an active ingredient in pharmaceutical compositions.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to a compound of the present invention, the pharmaceutical compositions can also contain other pharmaceutically active compounds or a plurality of compounds of the invention.

More particularly, a compound of the formula of the present invention also referred to herein as the active ingredient, may be administered for therapy by any suitable route including oral, rectal, nasal, topical (including transdermal, aerosol, buccal and sublingual), vaginal, parenteral (including subcutaneous, intramuscular, intravenous and intradermal) and pulmonary. It will also be appreciated that the preferred route will vary with the condition and age of the recipient, and the disease being treated.

In general, a suitable dose for each of the above-named compounds, is in the range of about 1 to about 100 mg per kilogram body weight of the recipient per day, preferably in the range of about 1 to about 50 mg per kilogram body weight per day and most preferably in the range of about 1 to about 25 mg per kilogram body weight per day. Unless otherwise indicated, all weights of active ingredient are calculated as the parent compound of the formula of the present invention, for salts or esters thereof, the weights would be increased proportionately. The desired dose is preferably presented as two, three, four, five, six or more sub-doses administered at appropriate intervals throughout the day. These sub-doses may be administered in unit dosage forms, for example, containing about 1 to about 100 mg, preferably about 1 to above about 25 mg, and most preferably about 5 to above about 25 mg of active ingredient per unit dosage form. It will be appreciated that appropriate dosages of the compounds and compositions of the invention may depend on the type and severity and stage of the disease and can vary from patient to patient. Determining the optimal dosage will generally involve the balancing of the level of therapeutic benefit against any risk or deleterious side effects of the treatments of the present invention.

Ideally, the prodrug should be administered to achieve peak concentrations of the active compound at sites of disease. This may be achieved, for example, by the intravenous injection of the prodrug, optionally in saline, or orally administered, for example, as a tablet, capsule or syrup containing the active ingredient. Desirable blood levels of the prodrug may be maintained by a continuous infusion to provide a therapeutic amount of the active ingredient within disease tissue. The use of operative combinations is contemplated to provide therapeutic combinations requiring a lower total dosage of each component antiviral agent than may be required when each individual therapeutic compound or drug is used alone, thereby reducing adverse effects.

While it is possible for the prodrug ingredient to be administered alone, it is preferable to present it as a pharmaceutical formulation comprising at least one active ingredient, as defined above, together with one or more pharmaceutically acceptable carriers, therefore, and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then if necessary shaping the product.

Formulations of the present invention suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented a bolus, electuary or paste.

A tablet may be made by compression or molding, optionally with one or more accessory ingredients. Compressed tablets may be prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Pharmaceutical compositions for topical administration according to the present invention may be formulated as an ointment, cream, suspension, lotion, powder, solution, paste, gel, spray, aerosol or oil. Alternatively, a formulation may comprise a patch or a dressing such as a bandage or adhesive plaster impregnated with active ingredients and optionally one or more excipients or diluents.

For diseases of the eye or other external tissues, e.g., mouth and skin, the formulations are preferably applied as a topical ointment or cream containing the active ingredient in an amount of, for example, about 0.075 to about 20% w/w, preferably about 0.2 to about 25% w/w and most preferably about 0.5 to about 10% w/w. When formulated in an ointment, the prodrug may be employed with either a paraffinic or a water-miscible ointment base. Alternatively, the prodrug ingredients may be formulated in a cream with an oil-in-water cream base.

If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the prodrug ingredient through the skin or other affected areas. Examples of such dermal penetration enhancers include dimethylsulfoxide and related analogues.

The oily phase of the emulsions of this invention may be constituted from known ingredients in a known manner. While this phase may comprise merely an emulsifier (otherwise known as an emulgent), it desirably comprises a mixture of at lease one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulphate.

The choice of suitable oils or fats for the formulation is based on achieving the desired cosmetic properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the prodrug ingredient. The prodrug ingredient is preferably present in such formulation in a concentration of about 0.5 to about 20%, advantageously about 0.5 to about 10% particularly about 1.5% w/w.

Formulations for rectal administration may be presented as a suppository with a suitable base comprising, for example, cocoa butter or a salicylate.

Formulations suitable for vaginal administration may be presented as suppositories, tampons, creams, gels, pastes, foams or spray formulations containing in addition to the prodrug ingredient, such carriers as are known in the art to be appropriate.

Formulations suitable for nasal administration, wherein the carrier is a solid, include a coarse powder having a particle size, for example, in the range of about 20 to about 500 microns which is administered in the manner in which snuff is taken, i.e., by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the prodrug ingredient.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more tissues. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily subdose, as herein above-recited, or an appropriate fraction thereof, of a prodrug ingredient.

It should be understood that in addition to the ingredients particularly mentioned above, the formulations of this invention may include other agents conventional in the art having regard to the type of formulation in question, for example, those suitable of oral administration may include such further agents as sweeteners, thickeners and flavoring agents.

Prodrugs and compositions of the formula of the present invention may also be presented for the use in the form of veterinary formulations, which may be prepared, for example, by methods that are conventional in the art.

Agricultural Applications

Some embodiments are useful in agriculture. Accordingly, this invention also provides a composition comprising the compound of this invention and a carrier, such as a solvent or agriculturally suitable carrier. In a further embodiment, the composition includes at least one chemical or biological pesticide, or both, as is conventionally used in the art.

For ease of application to plants or plant roots, the formulations can be processed into a formulation selected from the group consisting of a wettable powder, an aqueous suspension, an emulsifiable concentrate and a microencapsulated formulation.

Thus, the compounds of this invention can be used in a method for protecting or treating a plant or plant root from pathogenic infestations by applying an effective amount of the compound to the plant or root. In one aspect, the method further comprises applying at least one chemical or biological pesticide.

The following examples are intended to illustrate, but not limit the invention.

Experiment #1—Identification of iECTA Enzyme Targets

Alternative #1

The query method involves:

1. Go to WIT (“What Is There”) site on the Internet: At the time of the filing of this application, the WIT site was at the URL, http://wit.mcs.anl.gov/WIT2/CGI/search.cgi?user=.

2. Select “General Search,” check “All Enzymes,” and select “Match the Exact String”“.” (i.e. consisting only of a full stop). This will output all EC numbers in the microbial database when the maximum output table length is specified to be greater than the then-current maximum EC number. For example, as of the filing of the first priority application on Jul. 20, 2000, there were 3,546 enzymes listed in the EC database. The entire list of EC numbers could be output, therefore, by specifying the maximum output table length as any number greater than or equal to 3,546 (e.g., 10,000).

3. Select and copy all EC numbers and enzyme names to a Microsoft Word (or similar) document, and sort the EC numbers in order to get a useful list of names.

4. Paste the EC numbers and enzyme names into the KEGG form at: http://www.blast.genome.ad.jp/kegg-bin/mk point html?ec. This will search enzymes in the pathway database by EC numbers.

5. Select “Homo sapiens” from the pull-down menu and choose to “Display EC number(s) NOT found in the search.” This will output a list of enzymes identified by EC numbers, as well as a list of enzyme names. This initial output indicates the input enzymes on the KEGG metabolic map outlined in red that are characterized as NOT being present in human cells. Input enzymes that are present in human cells are outlined in red with a green fill. Because the descriptions of all genomes are incomplete at present, this is a list of candidate iECTA targets present in non-human species.

Alternative #2 (as shown in FIG. 2C):

1. Go to Genomes Online Homepage, ERGO, on the internet:

2. http://wit.integratedgenomics.com/IGwit/CGI/examine.cgi?user=

3. Select general search, select a target organism or homo sapiens select ORFS (open reading frames), and select match EC“ ” (i.e., EC space). This will output all EC numbers in the microbial database corresponding to the target organism when the maximum output table length is selected to be 10,000. In the example, the list for individual pathogenic organisms have been combined to give a list consisting of 8,162 open reading frames that have been annotated as enzymes with defined EC numbers. This list can be copied and pasted into the KEGG form as described in step 4 of Alternative #1 in order to obtain a metabolic map with pathogen enzymes outlined in red, and human enzymes filled with green.

Alternative #3 (as shown in FIG. 2C):

1. Go to WIT (What Is There) site on the internet:

2. http://wit.mcsanl.gov/WIT2/CGI/search.cgi?user=

3. Select general search, select a target organism, select ORFS (open reading frames), and select match EC“ ” (i.e., EC space). This will output all EC numbers in the microbial database corresponding to the target organism when the maximum output table length is selected to be 10,000. For example, the list for individual pathogenic organisms have been combined to give a list consisting of 8,162 open reading frames that have been annotated as enzymes with defined EC numbers. Alternatively, a separate list of EC numbers can be compiled for each organism individually by selecting a single organism at a time.

4. Open the file as a Microsoft Word (or similar word processing program), and process the text until the EC numbers are listed in a column separated by linefeed characters. This can be done (for example), by using the Replace function of the word processor to replace “EC” with “\hat{ }1EC” to list all EC numbers at the left margin, and to replace all whitespace.

5. Next, set the indentation to place all EC numbers in a column. Select the column, copy and paste to a new document. Delete “(EC” using the Replace function of the word processor program, delete all spaces, and replace all instances of “\hat{ }1\hat{ }1” with“\hat{ }1”. Likewise, replace all instances of “\hat{ }p\hat{ }p” with “\hat{ }p”.

6. Copy the resulting column of numbers to a Simpletext or other text based word processor file that is recognized by a PERL or other computer language interpreter, and name the file “target_ec_num”.

7. Go to the SRS data integration page maintained by the European Bioinformatics Institute currently at http:/srs6.ebi.ac.uk/srs6bin/cgi-bin/wgetz?-page+top+-newId. Use the SRS interface to query a database representing enzymes expressed in humans. For example, the BRENDA database can be downloaded in this way by querying for [Organism] Human|homo sapiens AND [EC number]*. The resulting list of EC numbers is most conveniently saved as a text file, opened in Microsoft Word (or similar word processing program) and processed as in steps 3) through 6) above; save the final text file as human_ec_num.

8. A list of enzymes occurring in the target organism, but not in humans, or other species or combination of species can be obtained by running a computer program written in PERL or other computer language. The program can be re-run to delete enzymes present in any number of databases by re-applying step 8) using another database. For example, the SwissProt database human enzymes can be subtracted as well as the BRENDA database. Alternatively, the SwissProt and BRENDA lists can be combined, and the program run just once. The following illustrative PERL program was used to obtain the list of enzymes set forth in step 8:

To delete known human enzymes, as represented by enzyme commission (EC numbers) from lists of enzyme commission numbers comprising a number of pathogenic microorganisms. In this example, the lists of EC numbers for pathogenic organisms and Homo sapiens were downloaded from the Integrated Genomics website (http://wit.integratedgenomics.com/GOLD/), the website for the European Bioinformatics Institute (http://www.ebi.ac.uk/genomes/).

| #!/usr/bin/perl; | |
|---|---|
| open (HUMAN_EC_NUM, “human_ec_num”); | |
| # input human EC numbers | |
| while (<HUMAN_EC_NUM>) { | |
| chomp; | |
| push (@human_ec_list, $_); #store the numbers in a list | |
| } | |
| open (TARGET_EC_NUM, “target_ec_num”); | |
| #input target EC numbers | |
| while (<TARGET_EC_NUM>) { | |
| chomp; | |
| push (@target_ec_list,$_); #store target EC numbers in a list | |
| } | |
| while (@human_ec_list) {#compare each human EC number with | |
| $a = pop (@human_ec_list); #each target EC number | |
| foreach (@target_ec_list) { | |
| if ($_eq $a) { | #if the the target EC number matches |
| s/$a/ERGO/; | #replace with the string ERGO |

```
}
}
}
print “ECTA EC numbers are: \n”;  #print the list to the screen
open (OUT,“>target_ec_numbers”);  #save the list to a file
foreach (@target_ec_list) {
  print (OUT $_);
  print (OUT “\n”);
  print “$_\n”;
}
close (OUT) ∥die “can't close”;
```

The output of the program in step 8 lists all ECTA targets, whether or not they are part of a recognized metabolic pathway; enzymes present in BRENDA (Homo sapiens and other mammals, in this instance) and SwissProt (Homo sapiens) can be indicated.

A list of enzymes organized into metabolic pathways can be obtained from the resulting total target_ec_num_ list by pasting this list into the KEGG website http://www.blast.genome.ad.jp/kegg/kegg2.html, selecting the organism homo sapiens, selecting Display EC/Compound/Gene(s) NOT found in the search, and clicking execute. ECTA enzymes that cannot be placed in a metabolic pathway by KEGG will be listed apart from those organized into metabolic pathways.

Alternative #4

1. Download the list of all existing EC numbers defined by the International Union of Biochemistry and Molecular Biology. For example, the current list can be obtained by going to the nomenclature site of the IUBMC at http://www.chem.qmw.ac.uk/iubmb/, and saving a text file containing a list of each of the six enzme categories, concatenating these files, then removing all characters from the file except the EC numbers using a wordprocessing program such as Microsoft Word.

2. “Dehumanize” the list of EC numbers by subtracting ERGO human EC numbers, BRENDA human EC numbers, and SwissProt human EC numbers by running the PERL program listed in Alternative #2 to delete human EC numbers.

3. Identify microbial enzymes by using the “dehumanized” EC number list to select “hits” in a file created by concatenating the annotated EC numbers and enzyme descriptions for target organisms (alternatively, each target organism can be analyzed individually). In this example, a file consisting of the EC numbers and descriptions comprising 51 microbial genomes representing human microbial pathogens is first catenated with the following PERL script:

#!/usr/bin/perl-w

#catenates text files listed below:

@ARGV=(“Yersinia pseudotubercul”, “Yersinia pestis”, “Vibrio cholerae El Tor N16961”, “Ureaplasma urealyticum”, “Treponema pallidum”, “Streptomyces coelicolor”, “Streptomyces coelicolor”, “Streptococcus pyogenes”, “Streptococcus pneumonia”, “Streptococcus mutans”, “Streptococcus equi”, “Staphylococcus aureus”, “Salmonella typhimurium”, “Salmonella typhi”, “Salmonella paratyphi”, “Salmonella enteritidis”, “Salmonella dublin”, “Saccharomyces cerevisia”, “Rickettsia prowazekii”, “Pseudomonas aeruginosa”, “Porphyromonas gingivalis”, “Pasteurella multocida”, “Neurospora crassa”, “Neisseria meningitidis ser. B ”, “Neisseria meningitidis ser. A ”, “Neisseria gonorrhoeae”, “Mycoplasma pneumoniae”, “Mycoplasma genitalium”, “Mycobacterium tuberculosis”, “Mycobacterium leprae”, “Mycobacterium bovis”, “Klebsiella pneumoniae”, “Helicobacter pylori”, “Helicobacter pylori J99”, “Haemophilus influenzae”, “Haemophilus ducreyi”, “Escherichia coli”, “Enterococcus faecium (DOE)”, “Enterococcus faecalis”, “Corynebacterium diphthe”, “Clostridium difficile”, “Clostridium acetobutyli”, “Chlamydia trachomatis D”, “Chlamydia trachomatis M”, “Chlamydia pneumoniae AR39”, “Chlamydia pneumoniae CWL029”, “Campylobacter jejuni”, “Borrelia burgdorferi”, “Bordetella pertussis”, “Bordetella bronchiseptica”, “Bacillus subtilis”);

open (OUT, “>outfile”); #Outfile is the catenated file
while (<>) {

print $_;
print OUT $_;

}
Close (OUT)∥die “can't close”;

4. Once the files containing EC numbers and enzyme descriptions for target microorganisms are catenated, the enzymes not occurring in humans can be selected by running another PERL program using the “dehumanized” EC numbers as input (EC numbers are converted from 1.1.1.1 to 1 1 1 1 format by replacing “.” with “_” before running this program):

#!/usr/bin/perl -w

#This program outputs EC Enzyme Descriptions for microorganisms, as shown in 5a

#The EC numbers represent enzymes that do not occur in humans

open (TARGET_EC_NUM, “Dehuman EC nums clean”);
open (OUT,“>All pathogen ECTA LIST”);
while (<TARGET_EC_NUM>) {
chomp;

push (@target_ec_list, $_);

}
%seen = ( );
foreach $item (@target_ec_list) {

push (@uniq, $item) unless $seen{$item}++;

}
@target_ec_list = @uniq;
@target_ec_list = reverse(@target_ec_list);
while (@target_ec_list) {

$a = pop (@target_ec_list);

open (PATHOGEN_EC_LIST,“KITTY EC LIST TEXT copy”);
while (<PATHOGEN_EC_LIST>) {
if(Λb$a\b/) {

print “$a $_”;
print OUT “$a $_”;

}
}
}
close (OUT) ∥die “can't close”;

The output of this program is given in FIG. 7A, which lists the EC numbers and descriptions of ECTA enzymes for each target organism. An abbreviated list consisting of all the EC number descriptions, but listing only one occurrence for each organism is shown in FIG. 7B, and consists of the 673 enzymes indicated by the following PERL script:

#!/usr/bin/perl -w
open (TARGET_EC_NUM, “Dehuman EC nums clean”);
open (OUT,“>All pathogen ECTA LIST”);
while (<TARGET_EC_NUM>) {
chomp;

push (@target_ec_list, $_);

}
%seen = ( );
foreach $item (@target_ec_list) {

push (@uniq, $item) unless $seen{$item}++;

}
@target_ec_list = @uniq;
@target_ec_list = reverse(@target_ec_list);
while (@target_ec_list) {

$a = pop (@target_ec_list);
$b = $a;

open (PATHOGEN_EC_LIST,“KITTY EC LIST TEXT copy”);

while (<PATHOGEN_EC_LIST>) {

if($a eq $b){
if(Λb$a\b/) {

print “$a $_”;
print OUT “$a $_”;
b = $_;

}
}
}
}
close (OUT) ∥ die “can't close”;

The methods of this invention provide the following unexpected advantages over prior art methods:

1. By taking the intersection of data sets from two different sources, a new data set is generated with useful properties that may contain enzymes or enzyme types that are present in pathogenic or undesirable microorganisms, but not present in uninfected or host cells.

2. Although the method makes use of an existing data set that returns enzyme names and EC numbers in response to a search tool, the use of a computer algorithm to return all enzyme names which identify potential iECTA enzyme targets, by EC number, in pathogenic organisms is an innovation that can be broadly applied for identifying pathogen or species targets for therapeutics development, or other applications (e.g., discriminating between yeast and bacteria, and pathogenic vs. nonpathogenic bacteria, plant pests vs. food plants).

3. Because the data sets for total microbial enzymes are subject to change as new enzyme genes are discovered, the method described above can continue to identify “new” iECTA targets.

Although the method has been illustrated by examples applicable to iECTA, the method is not limited to iECTA. For example, databases of enzymes elevated or expressed only in human cancer cells as compared to normal cells can be identified in an analogous fashion. For example, target enzymes for ECTA in cancer are expressed at elevated levels in tumor tissue as compared to normal tissue. Examples of such enzymes are given in Table 1(A). The difference in target enzyme expression between normal and tumor tissue allows for a positive therapeutic index to be achieved with ECTA compounds. Using this approach, the ECTA compound NB1011 (See U.S. Pat. No. 6,245,750) targets the enzyme thymidylate synthase (TS) which is overexpressed in cancer cells. Cytotoxicity of NB1011 is proportional to TS protein levels in model cell-based systems. TS inhibitors such as 5-fluorouridine have the reverse cytotoxicity profile since they are more toxic to the cells which express low amounts of the enzyme (Copur et al., 1995). In vivo studies have demonstrated efficacy against colon and breast cancer in animal models with little or no toxicity to the host.

Experiment #2—Analysis of Metabolic Networks

In selecting ECTA target enzymes it is useful to analyze the metabolic pathways and the networks of pathways in which particular potential target enzymes occur. For example, imidazolone propionase is shown here to be on the metabolic map relating to histidine degradation, in which boxes colored green (present as shaded in black and white reproductions) represent enzymes known (according to current information represented in the Kyoto Encyclopedia of Genes and Genomes) to occur in humans, and the proposed target enzyme, 3.5.2.7.

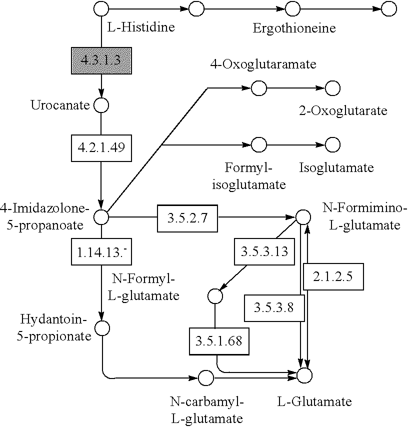

This map illustrates an aspect of ECTA enzyme selection, i.e. that it is desirable for the ECTA target enzyme to be connected to the network in such a way that there are no enzymes occurring in humans that are connected to the substrate (in this case, 4-imidazolone 5-propionate). This ensures that any ECTA substrate is unlikely to interact with a human enzyme. This condition is met in the above example, since 4.2.1.49 and 1.14.13 are both represented by unfilled boxes. EC numbers 4.3.1.3; 4.2.1.49; 1.14.13; 3.5.2.7; 3.5.1.68 and 3.5.3.8 have been identified with open reading frames in P. aeruginosa according to the WIT database, while only 4.3.1.3 was also found in humans.

Another example of selection of an intrinsic ECTA target by identification of an enzyme that catalyzes a favorable reaction type is methyl transferase. The methyl transferase enzyme thymidylate synthase has been shown to be amenable to development of ECTA substrates. A search of the WIT database for alternative related enzymes identified 2-demethylmenaquinone methyl transferase, EC 2.1.1.- as a potential intrinsic ECTA target.

In bacteria, the S-adenosylmethionine dependent 2-demethylmenaquinone methyl transferase catalyzes a step in the biosynthesis of menaquinone, or vitamin K₂. A number of pathogenic bacteria express this enzyme, including Escherichia coli, Enterococcusfaecalis, Haemophilus influenza, Mycobacterium leprae, Mycobacterium tuberculosis, Pseudomonasa aeruginosa, and Yersinia pestis. The reaction catalyzed by this enzyme involves the transfer of a methyl group, and is similar in this respect to thymidylate synthase, EC 2.1.1.45.

A tBLASTn search indicates that there is no human gene in the TIGR (The Institute for Genomic Research) human gene index that has a statistically significant degree of similarity to the S-adenosylmethionine dependent 2-demethylmenaquinone methyl transferase. This result is also consistent with the pathway data obtained from the Kyoto Encyclopedia of Genes and Genomes.

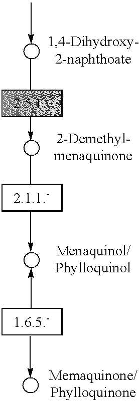

Enzyme EC 2.1.1.-is present in the target organism (Pseudomonas aeuruginosa), but not in humans. 2.5.1.-represents an enzyme that is present in humans. The pathway has no branches, thus the substrate 2-demethyl menaquinone is not expected to be a substrate for any human enzymes and is a useful target for development of ECTA compounds.

Experiment #3—Designing iECTA Compounds for Bacterial and Fungal Infections Using Enzymes in the Branched Chain Amino Acids Pathway

Using the above method, the enzymes acetolactate synthase (AcLS) or ketol-acid reductoisomerase (KARI) were identified as target enzymes. These two enzymes are preferred targets for iECTA because they are specific to the Branched Chain Amino Acid (“BCAA”) pathway which itself is specific to bacteria, fungi, and plants. Acetolactate synthase (AcLS) is the first enzyme in the pathway of branched chain amino acid (BCAA) synthesis. The active enzyme is present in bacteria, fungi, and plants, but not in mammals (Shaner and Singh (1997)). The absence of AcLS in animals allows effective use of AcLS inhibitors in herbicides, while avoiding toxicity to humans and animals (Shaner and Singh (1997) and Grandoni, et al. (1998)). Selectivity of enzyme function between disease causing organisms and animal or plant hosts can be used for designing iECTA compounds to fight bacterial and fungal infections. The product(s) may include toxins or antimetabolites that are preferentially generated by the bacteria or fungi.

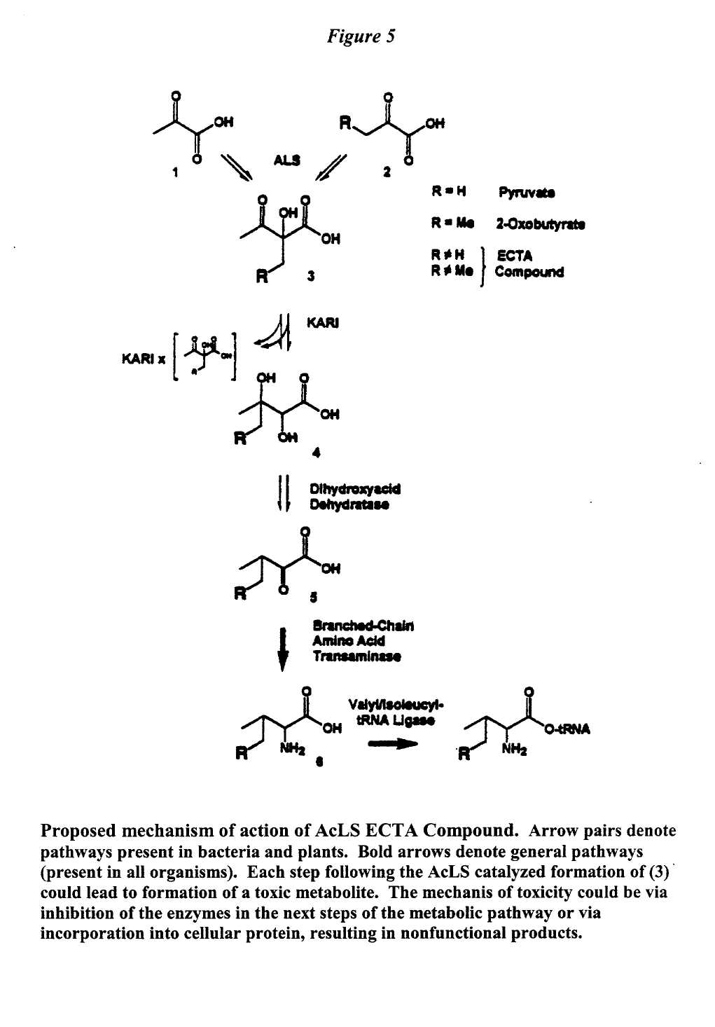

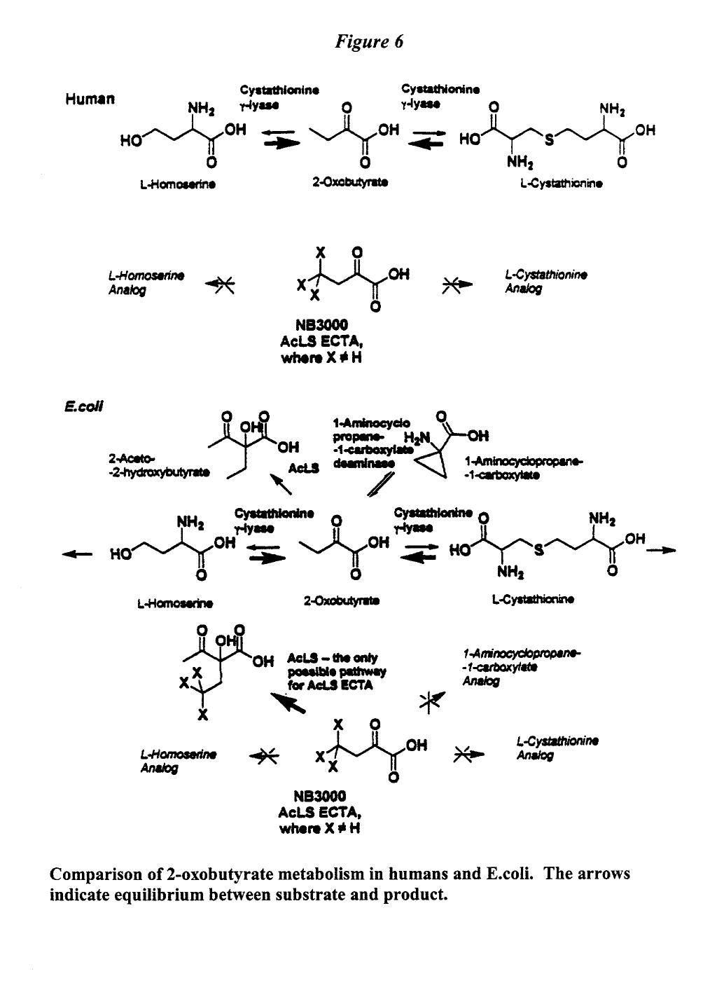

Acetolactate synthase (AcLS) is an α2β2 oligomer that consists of four subunits: two catalytic subunits with molecular weight of 60 kD and two regulatory subunits with molecular weight of 10-17 kDa (Pang and Duggleby (1999)). The enzyme catalyzes two similar reactions: the condensation of two pyruvate molecules to yield 2-acetolactate (FIG. 11), and the condensation of pyruvate with 2-oxobutyrate (2-OB, 2-ketobutyrate) to yield 2-aceto-2-hydroxybutyrate (FIG. 12). Thiamine pyrophosphate (TPP) is a cofactor in the reaction. Another cofactor, flavin adenine dinucleotide (FAD), is also necessary to maintain the catalytic structure of the enzyme (Shaner and Singh (1997)). The mechanism of AcLS reactivity with pyruvate and 2-OB is shown in FIG. 5. FIG. 6 is a comparison of 2-OB metabolism in E. coli and humans. AcLS is inhibited by BCAA feedback inhibition and a number of heterocyclic compounds, some of which are currently used as herbicides (FIG. 10) (Shaner and Singh (1997)). The crystal structure of AcLS is not yet available, but molecular modeling of the AcLS active site based on the structure of AcLS homologues, including pyruvate decarboxylase and pyruvate oxidase, has been completed (Ibdah, et al. (1996) and Chipman, et al. (1998)). Results show the existence of a deep substrate binding pocket with the cofactor binding at the bottom of the pocket. Site directed mutagenesis revealed that some herbicides (e.g., sulfonylurea; FIG. 10) bind close to the “entrance” of the pocket (Chipman, et al. (1998) and Chang and Duggleby (1998)). Branched chain amino acids inhibit the enzyme by an allosteric mechanism since they do not occupy the substrate binding site, but rather a distinct site between the two subunits (Shaner and Singh (1997)). AcLS inhibitors are effective as herbicides at low concentrations and have little toxicity to humans (Whitcomb (1999)).

KARI follows AcLS in the pathway of branched chain amino acid synthesis. It catalyzes isomerization of 2-acetolactate or 2-aceto-2-hyrdoxybutyrate with concomitant hydride transfer. The products of the reaction are 2,3-dihydroxy-isovalerate and 2,3-dihydroxy-3-methyl-valerate, respectively. The mechanism of the reaction is known in the art (Aulabaugh and Schloss (1990)). For catalysis, the enzyme requires Mg²⁺, which is involved in substrate binding, and NADPH, which is necessary to carry out the reductase function of KARI. KARI inhibitors include analogs of the transition state of the reaction (Halgand, et al. (1999)). Because the crystal structure of KARI is known (Halgand, et al. (1999)), this information can be used to aid the design of KARI iECTA compounds using simulated docking technology (Kirkpatrick, et al. (1999)).

Experiment #4—Designing iECTA Prodrugs to Target Enzymes

The following discussion is intended to illustrate, but not limit the invention. The natural substrates for AcLS are pyruvate or 2-oxobutyrate (2-OB). In designing possible iECTA molecules to target AcLS, the principal substrates and cofactors that are involved in the reaction were analyzed with respect to the criteria listed in FIGS. 2A and 2B and the metabolism of 2-OB in humans and E. coli were compared. Modifications of 2-oxobutyrate may be good candidates for AcLS ECTA compounds. 2-oxobutyrate (2-OB) is involved in few defined pathways. AcLS catalyzes condensation of 2-OB with the pyruvate in the first step in branched chain amino acid synthesis giving rise to Isoleucine. To enter this pathway a 2-OB derivative is required that is not significantly toxic by itself but can become toxic following reaction with AcLS. One possible fate of the product of the AcLS reaction with its ECTA molecule is that it can act as an inhibitor of ketol acid reductoisomerase (KARI), the next enzyme in the pathway of BCAA synthesis. Alternatively, ECTA compounds like derivatives of 2-OB may be incorporated into proteins by organisms with active AcLS. This would result in pathogen-specific metabolic poisoning of the pathogen (bacteria, yeast) and may also be effective for herbicidal activity. A summary of this analysis is given in Table 3, below.

TABLE 4
Possible Pyrimidyl Compounds to Be Used
as ECTA Compounds for AcLS

| | Potential | |
|---|---|---|
| | as an ECTA | |
| Reactant | Substrate | Rationale |
| Thiamine | Limited | Lack of specificity. TPP interacts with |
| pyrophosphate | | multiple enzymes, including enzymes |
| (TPP-cofactor) | | present in animals. Alternatives in TPP |
| | | could therefore impact host systems. |
| Branched chain | Limited | Natural product. More likely to inhibit |
| amino acids | | the reaction of AcLS. |
| AcLS | Limited | Not processed into product by AcLS. |
| inhibitors | | |
| (Herbicides): | | |
| Imidazolinones | | |
| Pyrimidylthio- | | |
| benzoates | | |
| Sulfonylamino- | | |
| carbonyl- | | |
| triazolinones | | |
| Sulfonylureas | | |
| Pyruvate | Limited | Good substrate since it undergoes |
| | | decarboxylation on the first step of |
| | | reaction. In the normal pathway, A) two |
| | | molecules of pyruvate are condensed in |
| | | the first step of the BCAA pathway |
| | | which gives rise to Valine; and B) one |
| | | molecule is combined with a molecule |
| | | of 2-oxobutyrate to give rise to |
| | | Isoleucine. However, it is a “common |
| | | substrate” in the cell, is involved in |
| | | many metabolic pathways (reacts with |
| | | at least 10 different enzymes including |
| | | enzymes present in mammals). |
| 2-oxobutyrate | A leading | Modifications of 2-oxobutyrate may be |
| (2-OB) | candidate as | good candidates for AcLS ECTA |
| | scaffold for | compounds. 2-oxobutyrate (2-OB) is |
| | AcLS ECTA | involved in few defined pathways. |
| | compound | AcLS catalyzes condensation of 2-OB |
| | | with the pyruvate in the first step in |
| | | branched chain amino acid synthesis |
| | | giving rise to Isoleucine. To enter this |
| | | pathway we need a 2-OB derivative |
| | | that is not significantly toxic by itself |
| | | but can become toxic following reaction |
| | | with AcLS. One possible fate of the |
| | | product of the AcLS reaction with its |
| | | ECTA molecule is that it can act as an |
| | | inhibitor of ketol acid reductoisomerase |
| | | (KARI), the next enzyme in the |
| | | pathway of BCAA synthesis. |
| | | Alternatively, ECTA compounds like |
| | | derivatives of 2-OB may be |
| | | incorporated into proteins by organisms |
| | | with active AcLS. This would result in |
| | | pathogen-specific metabolic poisoning |
| | | of the pathogen (bacteria, yeast) and |
| | | may also be effective for herbicidal |
| | | activity. |

Such iECTA compounds will have therapeutic antimicrobial (and possible herbicidal) properties. Proposed pathways of metabolism for AcLS iECTA compounds derived from 2-OB are compared for humans and bacteria as shown in FIG. 6. In humans and bacteria, cystathionine-2-lyase catalyzes 2-oxobutyrate conversion to L-homoserine and L-cystathionine. In bacteria, two additional enzymatic reactions can occur. These are reactions of 2-OB with 1-aminocyclopropane-1-carboxylate deaminase and acetolactate synthase. The structure of a potential AcLS iECTA compound is disclosed infra. The tri-substitution of carbon-4 of 2-OB, where carbon-4 is CX₃ and where X≠H, is one of the key features in this design because it channels the iECTA compound toward reaction with AcLS, and prevents its reaction with cystathionine-2-lyase or 1-aminocyclopropane-1-carboxylate deaminase.

Once the iECTA compound is processed by AcLS, the fate of the product of the reaction may be different from that of the natural substrate. The AcLS iECTA product may:

1. Bind to KARI and be converted to the rearranged and reduced product. This product could be toxic, or become transformed to a toxin following a subsequent reaction.

2. It may not bind to KARI, but rather accumulate as a “dead-end” product and eventually starve the cells of pyruvate.

3. Be incorporated into cellular polypeptides, thereby leading to the formation of dysfunctional proteins.

The design of candidate KARI iECTA compounds is based upon the same rationale as the design of AcLS iECTA compounds. In this case, the proposed scaffold is 2-aceto-2-hydroxybutyrate.

Either AcLS or KARI will utilize substrates and convert them to antimetabolites targeting multiple enzyme pathways.

Experiment #5—Synthesis of iECTA Compounds

This invention also provides compounds useful as AcLS and KARI iECTA compounds. In one aspect, the compounds have the structure:

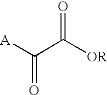

wherein A is a substituted or unsubstituted phenyl ring; or a substituted >C═C< more preferably —CH═CH—, substituents can include a substituted or unsubstituted aromatic or heteraromatic ring, more preferably a substituted or unsubstituted phenyl ring; or

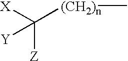

wherein n is 0 or an integer from 1 to 6, more preferably n is 0, 1, or 2, most preferably n is 0;

wherein X is H or halogen, more preferably halogen; Y is H or halogen, more preferably halogen; and Z is any of H; halogen; CF₃; aliphatic group; substituted or unsubstituted aromatic or heteraromatic ring, more preferably phenyl ring; substituted or unsubstituted aromatic carbonyl or heteraromatic carbonyl, more preferably substituted or unsubstituted benzoyl.

In all cases R═H, a pharmaceutically acceptable cation, or an aliphatic substituent, more preferably methyl or ethyl.

In another aspect, the compounds have the structure:

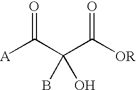

wherein A is a substituted or unsubstituted phenyl ring or a substituent having the structure:

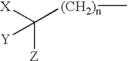

wherein n is 0 or an integer from 1 to 6, and more preferably 0, 1 or 2, and most preferably 0; wherein X is H or halogen, and more preferably halogen; wherein Y is H or halogen, and more preferably halogen; wherein Z is H; halogen; CF₃; aliphatic group; substituted or unsubstituted aromatic or heteraromatic ring, and more preferably phenyl ring; substituted or unsubstituted aromatic carbonyl or heteraromatic carbonyl, more preferably substituted or unsubstituted benzoyl.

B is a substituted or unsubstituted phenyl ring or a substituent having the structure:

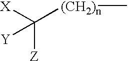

wherein n is 0 or an integer from 1 to 6, and more preferably 0, 1 or 2, and most preferably 0; wherein X is H or halogen, and more preferably halogen; wherein Y is H or halogen, and more preferably halogen; wherein Z is H; halogen; CF₃; aliphatic group; substituted or unsubstituted aromatic or heteraromatic ring, and more preferably phenyl ring; substituted or unsubstituted aromatic carbonyl or heteraromatic carbonyl, more preferably substituted or unsubstituted benzoyl.

In all cases R is H, a pharmaceutically acceptable cation, or an aliphatic substituent, more preferably methyl or ethyl.

Biological activity similar to iECTA compounds for AcLS and KARI has not been ascribed to any known drugs. However, the literature has provided a synthetic protocol for a possible candidate-compound. (Wakselman and Tordeuz (1982)). This paper describes synthesis of 3,3,3-trifluoropropriaonic and 4,4,4-trifluoro-2-ketobutyric acids. This synthetic protocol does not describe the synthesis of all compounds of the class identified above, but is easily adapted by those of skill in the art for this purpose.

EC 3.1.3.15 Histidinol Phosphatase

Histidinol phosphatase is found on the histidine biosynthetic pathway and is found in bacteria and yeast, but not in mammals. Mechanism is simply water hydrolysis of a phosphate group.

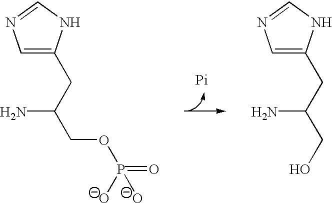

Histidinol Phosphatase iECTA Substrate

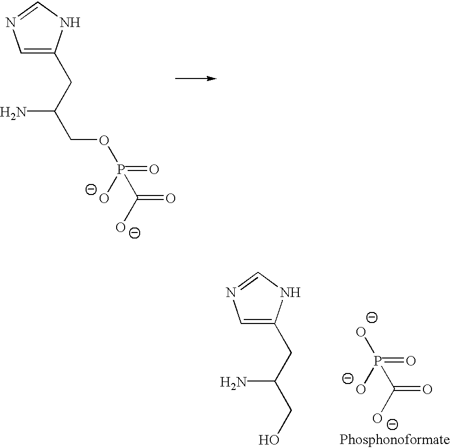

A toxophore can be substituted for the phosphate group in the substrate to create an ECTA substrate. In the example above, the DNA polymerase inhibitor phosphonoformate is used as an example of a toxophore.

Synthesis of Histidinol Phosphatase iECTA Substrate Histidinol phosphatase iECTA

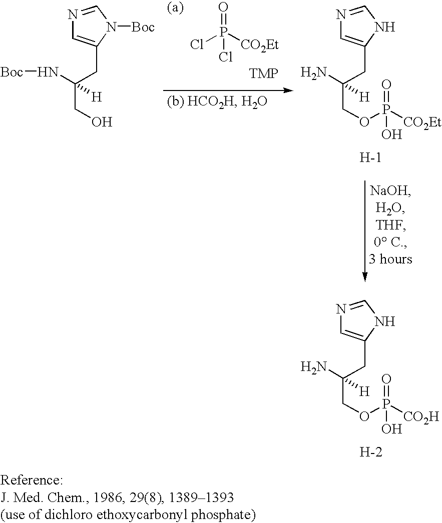

Compound H-1

To 1 gram (5.2 mmol) of ethoxycarbonyl phosphonic dichloride (J.Med Chem., 1986, 29(8), 1389-1393) dissolved in 10 mL of trimethylphosphate, and cooled to 0° C., is added 1 gram (2.9 mmol) of dry bis-N-tBoc-histidinol. The reaction is allowed to stir for 1 hour at 0° C., and then it is poured slowly into 25 mL of anhydrous diethyl ether with stirring. The product is separated by decantation and washed twice with 5 mL anhydrous diethyl ether. The crude product is dried under vacuum and dissolved in 5 mL of anhydrous formic acid at 0° C., allowed to warm up to room temperature and stirred for 2 hours, then heated gently at 50° C. for 30 minutes. Most of the formic acid is evaporated in vacuo at 30° C., then water is added, and the product evaporated to dryness under reduced pressure. The product is purified on a Dowex 2 (X8, 200-400 mesh, C1⁻ form) column by elution with water and elution with a gradient of 0.0-0.15 M LiCl. The water is evaporated and ethanol is added. The lithium salt of product H-1 is isolated after partial evaporation of the ethanol.

Compound H-2

Compound H-1 is treated with 1.5 eq. of NaOH in aqueous THF at 0° C. for 3 hours. After evaporation of the solvent under reduced pressure, the product is purified as above to furnish the lithium salt of Compound H-2.

EC 4.2.1.9 Dihydroxyacid Dehydratase

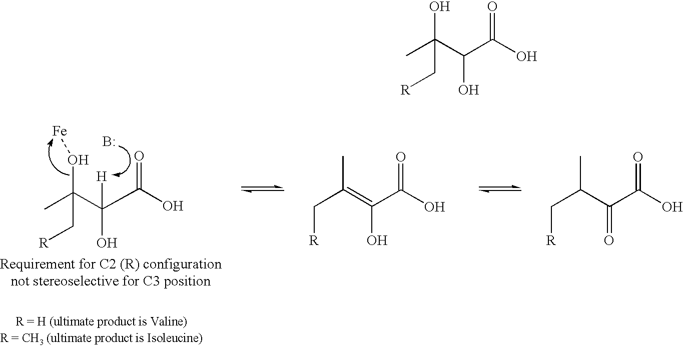

Dihydroxyacid dehydratase (DHAD) is an enzyme found on the branched chain amino acid biosynthetic pathway. The enzyme mechanism and substrate SAR has been well characterized by substitutions at C3 (See Pirrung et al. and Armstrong et al.).

Also, DHAD has been shown to be the target for the bacteriostatic effects of 4,7-dicyanobenzofarazan (See Takabatake et al.)

The DHAD ECTA substrate shown below is designed to generate a very reactive alkylating agent upon activation by the enzyme. DHAD iECTA Substrate

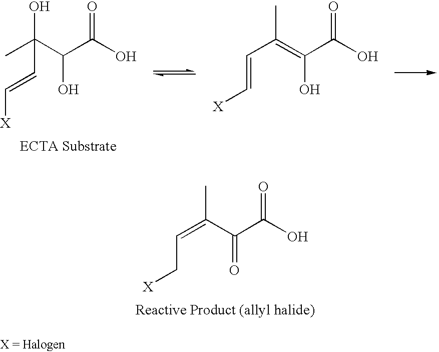

Synthesis of DHAD iECTA Substrate

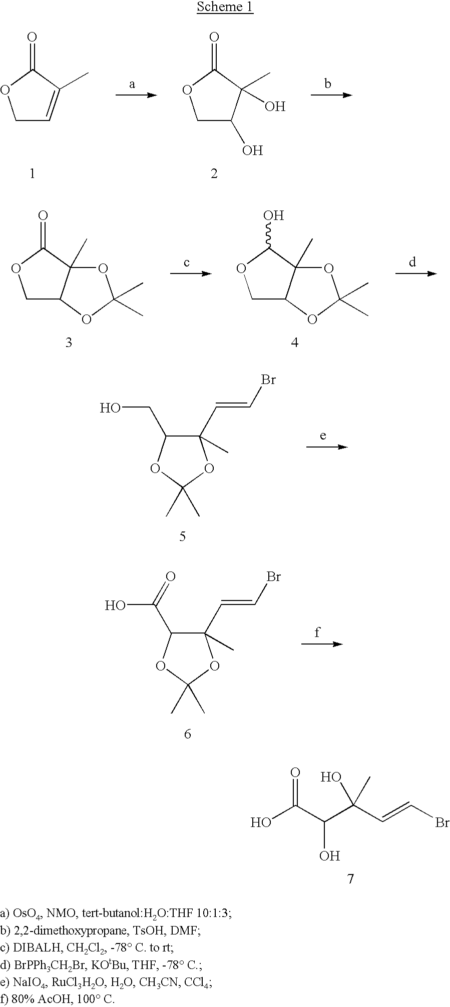

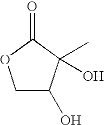

Dihydro-3,4-dihyroxy-3-methyl-2 (3H)-furanone (2)

To a mixture of N-methylmorpholine-N-oxide (36 mmol), tert-butanol (400 mL), H₂O (40 mL), THF (120 mL), and OsO₄ (0.39 mmol) is added 3-methyl-2(5H)-furanone (1) (34 mmol). The reaction is stirred overnight. A slurry of 6 g of sodium hydrosulfite, 8 g of Florisil® and 27 mL of water is added to the reaction mixture. The mixture is stirred and filtered. The filtrate is neutralized to pH 7 with 1 N H₂SO₄. The THF is evaporated in vacuo and the remaining mixture is acidified to pH 2 with 1 N H₂SO_{4.} The solution is extracted with 3×150 mL of ethyl acetate. The organic layers are combined, dried over Na₂SO₄, and filtered. The solvent is evaporated in vacuo. (See VanRheenen, et al.)

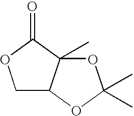

2-Methyl-2,3-O-isopropylidene-erythoronolactone (3)

2,2-Dimethoxypropane (84.7 mmol) and p-toluenesulfonic acid monohydrate (catalytic) is added to a solution of 2 (84.7 mmol) and dimethylformamide (21.2 mL). The reaction mixture is stirred overnight. The reaction is quenched with 30 mL of H₂O. The water layer is extracted with 3×50 mL of ethyl acetate. The combined organic layers are washed with 2×30 mL of water and with 5×30 mL of brine or until the organic layer is clear. The organic layer is dried over Na₂SO₄ and filtered. The solvent is evaporated in vacuo. (See Evans et al. and Lipshutz et al.)

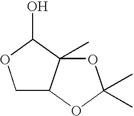

2-Methyl-2,3-0-isopropylidene erythrose (4)

All glassware is flame dried and the reaction is performed under argon gas. A flask is charged with 3 (10.0 mmol) and CH₂Cl₂ (31.3 mL) and cooled to −78° C. in an acetone-dry ice bath. A 1.0 M solution of DIBALH in THF (15.6 mmol) is added dropwise down the sides of the flask. The reaction mixture is stirred at −78° C. for 3 hours. The reaction mixture is warmed to room temperature overnight. The reaction mixture is cooled to 0° C. in an ice bath and 5 mL of methanol is added to quench the reaction mixture. To a mixture of 1: 1 water ethyl acetate (150 mL each) is added to the reaction mixture. The aqueous layer is acidified to pH 3 with 5% H₂SO₄. The phases are separated and the aqueous layer is extracted with 2×75 mL ethyl acetate. The combined organic layers are dried over Na₂SO₄, filtered, and the solvent is evaporated in vacuo. (See Gypser et al. and Cohen et al . . . )

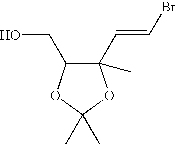

All glassware is flame dried and the reaction is performed under argon gas. A flask charged with (bromomethyl)triphenylphosphonium bromide (12.6 mmol) and THF (22 mL) is cooled to −78° C. in an acetone-dry ice bath and a 1 M solution of potassium tert-butoxide in THF (12.6 mmol) is added dropwise. A solution of 4 (4.2 mmol) in THF (2.2 mL) is added dropwise. The reaction mixture is stirred for 1 hour at −78° C. The cooling bath is removed and the reaction mixture is left overnight. The reaction mixture is quenched with 50 mL of water. The water layer is extracted with 3×50 mL of diethyl ether. The organic layers are combined and washed with 1×100 of brine. The organic layer is dried over MgSO₄, filtered, and the solvent is evaporated in vacuo. (See Gypser, et al. and Dötz, et al.)

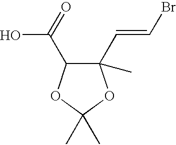

To a flask charged with water (3 mL), CCl₄ (2 mL), and acetonitrile (2 mL), is added NaIO₄ (4.7 mmol), ruthenium(III) chloride hydrate (2.2 mol percent), and 5 (1.1 mmol). The reaction mixture is stirred at room temperature for 1 hour. Diethyl ether (20 mL) is added to the reaction mixture which is then stirred for 10 minutes. The solution is filtered and the solids are washed with diethyl ether. The solvent is evaporated in vacuo. (See J. Org. Chem.)

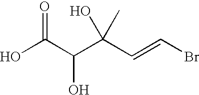

Isopropylidene 6 (1.0 mmol) is dissolved in 80% acetic acid (5 mL) and heated at 100° C. for 1.5 hours. The reaction mixture is cooled and the solvent is evaporated in vacuo. (See Lewbart, et al. and Hanessian, et al.).

EC 4.1.3.27 Anthranilate Synthase

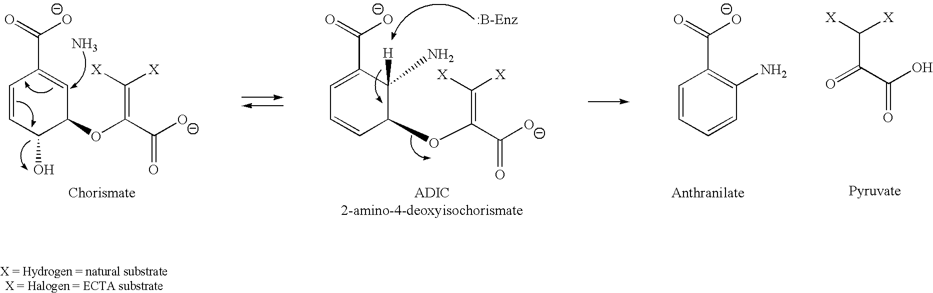

X Hydrogen=natural substrate

X=Halogen=ECTA substrate

Chorismate is the branch point for the biosynthesis of several natural products. The reaction shown above is the first step down the tryptophan biosynthetic pathway where chorismate is converted to anthranilate and pyruvate. By substituting halogens for one or both hydrogens as shown above, a very potent alkylating agent can be produced, di- or mono-halo-pyruvate. This can be the basis for several other ECTA substrates. The synthesis of a chorismate based ECTA substrate is shown below:

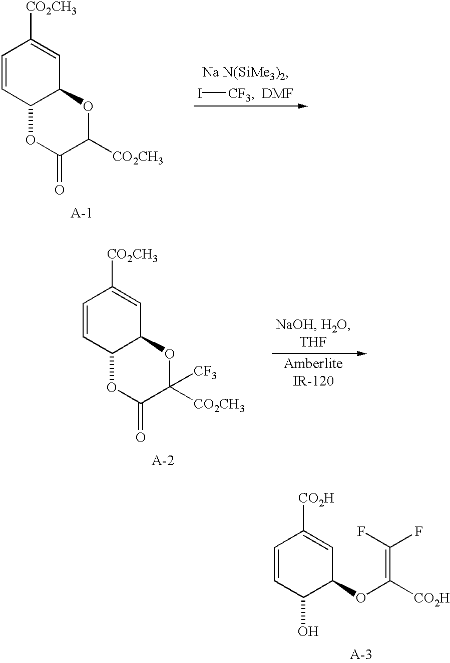

Synthesis of Anthranilate Synthase ECTA Substrate

Compound A-1

Hexamethyldisilazane (0.350 g, 2.16 mmol) is added to a suspension of oil free NaH (0.048 g, 2.00 mmol) (pre-washed with petroleum ether) in 5 mL of anhydrous DMF. When H₂ evolution ceases, a solution of lactone A-1 (See Ganem et al.) (0.536 g, 2.00 mmol) in 5 mL DMF is added. After stirring 15 minutes at room temperature, a solution of trifluoromethyl iodide (0.800 g, 4.10 mmol) in 5 mL DMF is added, and the reaction is allowed to proceed at room temperature for 3 hours. The reaction is then poured into saturated NaCl solution and extracted with ethyl acetate (2×25 mL). The combined organic layers are washed with saturated NaCl (2×25 mL), dried over anhydrous Na₂SO₄, and the solvent evaporated under reduced pressure to furnish crude A-2, which is processed as is in the next step.

Compound A-3

The crude product from the alkylation step is dissolved in 10 mL of ice-cold THF and 2.0 equivalents of aqueous 0.01 M NaOH are added. The reaction is allowed to proceed at 0° C. for 3 hours. After 3 hours, the reaction is stirred gently with Amberlite IR-120 resin (See Berchtold, et al.), filtered, and the solvent evaporated under reduced pressure. Compound A-3 is purified by recrystallization from ethyl acetate-hexane.

EC 4.1.3.12 Isopropylmalate Synthase

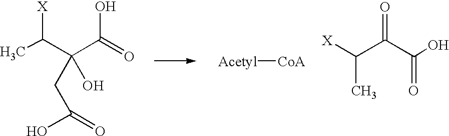

X=CH₃=natural substrate

X=Halogen=ECTA substrate

The reaction shown above is on the branched chain amino acid (BCAA) biosynthetic pathway. By substituting a halogen for one of the methyl groups as shown above, a very potent alkylating agent can be produced, 3-halo-2-oxobutanoate.

Synthesis of 2-Isopropylmalate Synthase iECTA

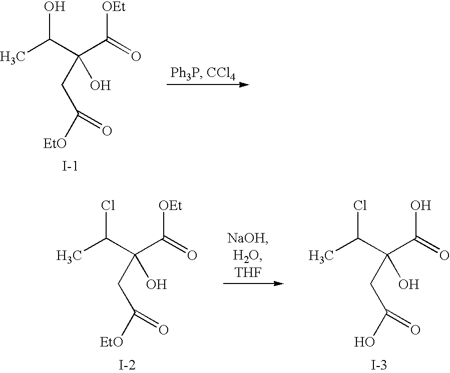

To a mixture of 1 gram of dihydroxy diester I-1 (See Zhdanov et al.) and 2.5 grams of anhydrous triphenyl phosphine is added 20 mL of anhydrous CCl₄, and the reaction is refluxed for 15-30 minutes (See Tetrahedron). The solvent is then evaporated to dryness under reduced pressure. The residue is extracted twice with 25 mL of diethyl ether, and the combined fractions are evaporated under reduced pressure. The resulting crude chloroester I-2 is hydrolyzed to the corresponding dicarboxylic acid by stirring with 2.0 equivalents of aqueous NaOH in THF at 0° C. for 3.5 hours. The reaction is then acidified with dilute HCl, and the product is obtained by extraction with two 25 mL portions of ethyl acetate. The ethyl acetate fractions are combined, dried with anhydrous Na₂SO₄, fitered and the solvent is removed under reduced pressure. The product I-3 is purified by chromatography on silica gel using EtOAc/Hexane/HOAc.

EC 5.3.3.4 Muconolactone Δ-Isomerase

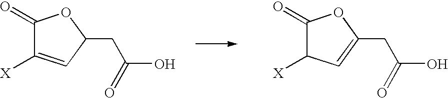

X=Hydrogen=natural substrate

X=Halogen=ECTA substrate

The reaction shown above is on the phenylalanine metabolism pathway. The enzyme converts a stable vinyl-halo ester into a very reactive (alkylator) allyl halide species.

Muconolactone Δ-Isomerase ECTA Substrate

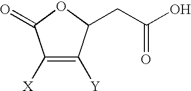

KNOWN COMPOUNDS

X=Cl, F; Y=H

X=Br, Cl; Y=OH, CH₃

See McKague, Vollmer, et al., Freer, et al., Patrick et al., Bloomer, et al., and Syendsen, et al.

EC 6.3.2.12 Dihydrofolate Synthase

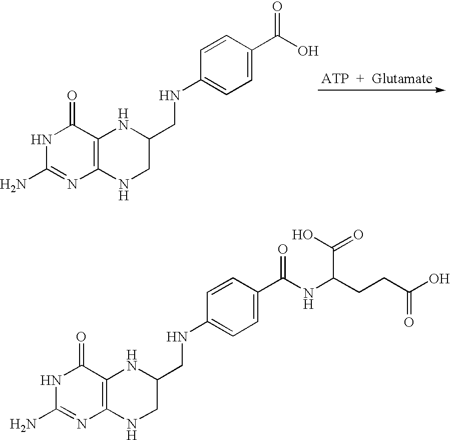

The reaction shown above is the final step in folic acid biosynthesis where a glutamate is conjugated to the dihydropteroic acid substrate. Most microorganisms produce their own folic acid, whereas it is an essential vitamin for humans because we lack this biosynthetic pathway right up through this step. Antifolates have been used for both cancer chemotherapy as well as for microbial infections, but they are only potent after glutamate conjugation (See Goodman and Gillman (1996) THE PHARMACOLOGICAL BASIS OF THERAPEUTICS, 9^{th} edition, McGraw-Hill). We can take advantage of the above reaction for ECTA by delivering a pteroic acid analogue of an antifolate and allowing the microbial dihydrofolate synthase to attach the first glutamate. The pteroic acid itself should not be toxic to the host.

iECTA Substrate for Dihydrofolate Synthase:

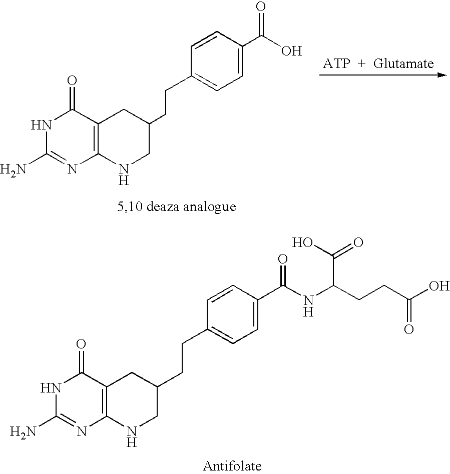

The 5,10-dideazapteroic acid is a known compound, and its synthesis has been published. The 5,10-dideazafolate is an experimental antifolate (Id.) (See also Degraw et al. and Taylor et al.).

Experiment #6—Biological Confirmation for Selecting a Candidate AcLS or KARI iECTA Prodrug

Salmonella typhimurium, Escherichia coli or other bacteria or fungi are used as test cells. Two phenotypes are employed. One strain is normal for acetolactate synthase (AcLS) and the other is deficient. Such strains have been previously described, e.g., Shaw, et al. (1980) and Weinstock, et al. (1992) and can be obtained from the American Type Culture Collection, the E. coli Genetic Stock Center (Yale University), the Salmonella Genetic Stock Center (University of Calgary, Canada), and other sources. The AcLS-negative strains are generally referred to as ilv⁻ because they are dependent upon added isoleucine and valine for growth. The ilv⁻ mutant strains will be compared to the normal parent strains (ilv⁻) for sensitivity to candidate compounds. Strains that express the active form of AcLS will be able to transform an AcLS iECTA compound into a cytotoxic moiety. For this reason, the normal strains will be more sensitive to a successful AcLS compound than will the mutant ilv⁻ strains. Similar assays can also be performed on mammalian cells to determine the degree of specificity for AcLS-producing bacteria or fungi.

Assays are performed on agar plates or in liquid media containing the appropriate nutrients (Miller (1972)). Inhibition of growth of ilv⁺ strains is measured by decreased colony formation on agar plates containing a potential AcLS activated prodrug, or decreased growth rate in liquid culture containing the candidate drug (Minimal Inhibitory Concentration, MIC.) Utility is further demonstrated by performing these assays comparing the candidate AcLS iECTA compounds with known antibiotics versus pathogens. Similar growth assays can be performed to test the utility of potential compounds on yeast and other potential pathogens using methods appropriate for these eukaryotic organisms, as described by Spector, et al. (1998). Additional tests are performed to demonstrate minimal toxicity vs. normal animal or human cells. These tests are done as described by Sugarman, et al. (1986). A satisfactory result will be at least 10-fold, and preferably one hundred or one thousand fold greater sensitivity of pathogen (such as bacteria or yeast) to AcLS iECTA compounds as compared to animal or human cells.

Experiment #7—Prodrugs Designed To Target iECTA Enzymes

Using the methods described above, the following iECTA prodrugs and enzyme prodrug systems are provided.

When the enzyme is a member of the subgroup 1.1, the compound has the structure: R—CHOH—X-Toxin, wherein X is selected from the group consisting of O, S, and NH.

When the enzyme is a member of the subgroup EC 1.2, the prodrug is a compound of the structure: R—(C═O)—X-Toxin, wherein X is O or S.

When the enzyme is a member of the subgroup EC 1.3, the prodrug is a compound of the structure: R1R2—CH—CHR3—CH2—X-Toxin, wherein R1, R2 and R3 are unspecified, and wherein X is O or S.

When the the enzyme is a member of the subgroup EC 1.4 or 1.5, then the prodrug is a compound of the structure:

R1—CH(—NH-Toxin)-R2

or

R1—C(═NH)—X-Toxin;

wherein X is selected from the group O, S and NH, with the proviso that when X is S or O, the amine (NH) is not an amide.

When the enzyme is a member of the subgroup EC 3.1.1.10, the prodrug is a compound having either structure:

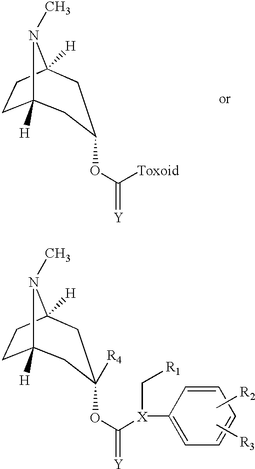

wherein Y is selected from the group consisting of O, S, Se, and NR;

wherein R is unspecified;

wherein X is selected from the group consisting of C and N; and

wherein each of R1, R2, R3 and R4 is independently the same or different and is a toxoid, or is unspecified. The “toxoid” is directly linked toxoid or connected through a linker (i.e., self-immolative).

When the enzyme is a member of the subgroup EC 3.1.1.11, the prodrug is a compound of the structure:

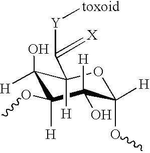

wherein X is selected from the group consisting of O, S, Se, and NR;

wherein R is unspecified; and

wherein Y is selected from the group consisting of O, S and NR, wherein R is unspecified. Toxoid is either toxoid or linker-toxoid.

When the enzyme is a member of the subgroup EC 3.1.1.17, the prodrug is a compound of the structure:

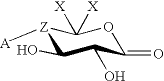

wherein A, X, and Y are independently the same or different and is selected from the group consisting of a toxoid, CH₂OH or CH₂OPO₃;

wherein Z is selected from the group consisting of CH2, N, NR, O, S and Se; and

wherein R is unspecified.

When the enzyme is a member of the subgroup EC 3.1.1.20, the prodrug is a compound of the structure:

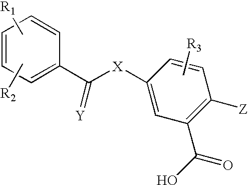

wherein R1, R2, R3 are independently the same or different and are unspecified; and

wherein Z is a linker and/or a toxoid.

When the enzyme is a member of the subgroup EC 3.1.1.24, the prodrug is a compound of the structure:

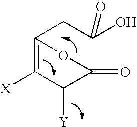

wherein X is a halide or hydrogen; and

wherein Y is a linker-toxoid or a toxoid.

When the enzyme is a member of the subgroup EC 3.1.1.29, the prodrug is a compound of the structure:

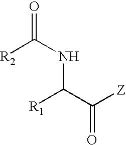

wherein each of R1 and R2 are independently the same or different and is selected from the group consisting of an amino acid, an amino acid side chain, a toxoid, a linker, or a peptide; and

wherein Z is selected from the group consisting of a toxoid which may have RNA like structure or be RNA or a nucleic acid analog.

When the enzyme is a member of the subgroup EC 3.1.1.32, the prodrug is a compound of the structure:

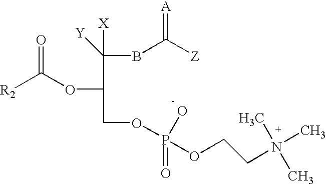

wherein R2 is a fatty acid;

wherein X, Y and Z are independently the same or different and is selected from the group consisting of a toxoid, toxoid linker, O, S, and NR, wherein R is unspecified; and

wherein A and B are independently the same or different and is selected from the group consisting of O, S, and NR wherein R is unspecified.

When the enzyme is a member of the subgroup 3.1.1.45, the prodrug is a compound of the structure:

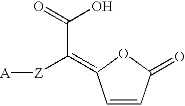

wherein A and Z are independently the same or different and is selected from the group consisting of toxoid, toxoid-linker, a halogen and a heteroatom.

When the enzyme is a member of the subgroup 3.1.1.57, the prodrug is a compound of the structure:

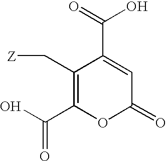

wherein Z is selected from the group consisting of toxoid and toxoid-linker.

When the enzyme is a member of the subgroup 3.1.1.61, the prodrug is a compound of the structure:

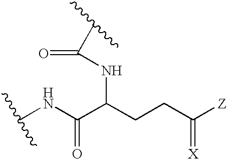

wherein Z is selected from the group consisting of toxoid and toxoid-linker; and

wherein X is selected from the group consisting of O, S, and NR, wherein R is unspecified.

When the enzyme is a member of the subgroup 3.1.2.1, the prodrug is a compound of the structure:

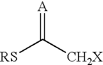

wherein RS is Coenzyme A (“CoAS”) or a variable thiol including smaller analogs of CoAS;

wherein A is selected from the group consisting of O, S, and NR, wherein R is unspecified; and

wherein X is Cl, Br, I and F.

When the enzyme is a member of the subgroup 3.1.2.12, the prodrug is a compound of the structure:

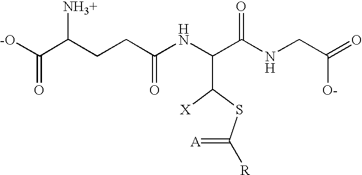

wherein X is H or a toxoid;

wherein A is selected from the group consisting of O, S, and NR;and

wherein R is selected from the group consisting of H, a halomethyl and a toxoid.

When the enzyme is a member of the subgroup 3.1.2.14, the prodrug is a compound of the structure:

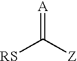

wherein R is selected from the group consisting a simple or complex thiol and ACP or acyl carrier protein;

wherein A is O, S, and NR, wherein R is unspecified; and

wherein Z is selected from the group consisting of a toxoid, a toxoid-linker, and a fatty acid analog having antibacterial/antifungal/antimicrobial properties.

When the enzyme is a member of the subgroup 3.1.3.10, the prodrug is a compound of the structure:

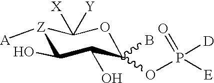

wherein A, B, X and Y are independently the same or different and each is selected from the group consisting of toxoid, CH2OH, CH2OPO3 and H;

wherein Z is selected from the group consisting of CH2, N, O, S, SE or NR, wherein R is unspecified; and

wherein D and E are independently the same or different and is selected from the group consisting of OH, NHCH₂CH₂Cl and SCH₂CH₂Cl.

When the enzyme is a member of the subgroup 3.1.3.12, the prodrug is a compound of the structure:

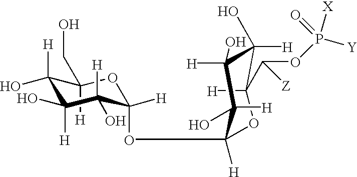

wherein Z is selected from the group consisting of a toxoid, H and a toxoid-linker; and

wherein X and Y are independently the same or different and is selected from the group consisting of OH, NHCH₂CH₂Cl and SCH₂CH₂Cl.

When the enzyme is a member of the subgroup 3.1.3.15, the prodrug is a compound of the structure:

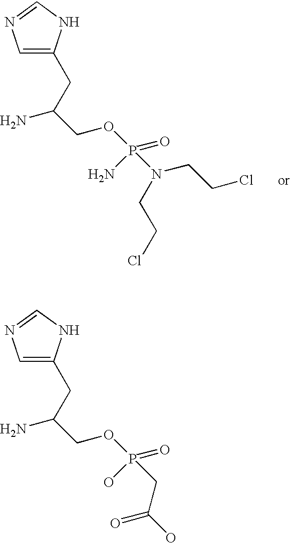

When the enzyme is a member of the subgroup 3.1.3.X, wherein X is 18 or 27, the prodrug is a compound of the structure:

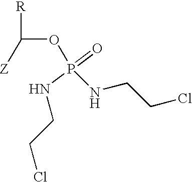

wherein Z is selected from the group consisting of H, a toxoid and toxoid-linker.

When the enzyme is a member of the subgroup 3.1.4.14, the prodrug is a compound of the structure:

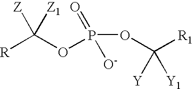

wherein Y, Y1, Z and Z1 is a toxoid.

When the enzyme is of the subgroup 3.1.4.16, the prodrug is a compound having the structure:

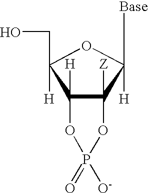

wherein the base is selected from the group consisting of adenine, tyrosine, guanine, cytosine and uracil; and

wherein Z is a toxoid.

When the enzyme is of the subgroup 3.1.5.1, the prodrug is a compound having the structure:

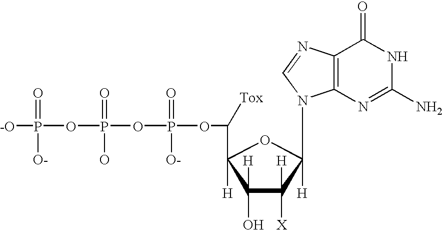

wherein X is H or OH; and

wherein Tox is a toxoid.

When the enzyme is of the subgroup 3.1.6.1, the prodrug is a compound having the structure:

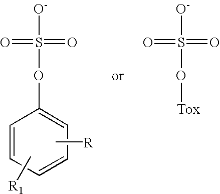

wherein R and R1 is a toxoid or linker-toxoid; and

wherein Tox is a toxoid.

When the enzyme is of the subgroup 3.1.7.2, the prodrug is a compound having the structure:

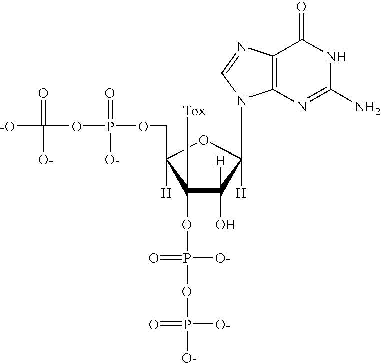

wherein Tox is a toxoid.

When the enzyme is of the subgroup 3.1.11.1, the prodrug is a compound having the structure:

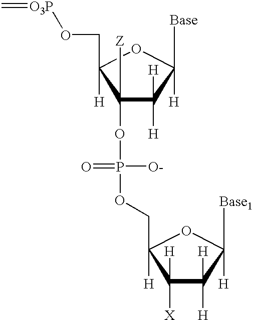

wherein the base is selected from the group consisting of adenine, tyrosine, guanine, cytosine and uracil;

wherein Base 1 is a toxoid;

wherein Z is selected from the group consisting of toxoid and toxoid-linker; and

wherein X is OH or a phosphate.

When the enzyme is of the subgroup 3.1.11.5, the prodrug is a compound having the structure:

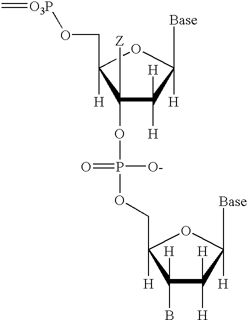

wherein Base is selected from the group consisting of adenine, tyrosine, guanine, cytosine and uracil;

wherein B is a phosphate or a DNA small oligonucleotide; and

wherein Z is a toxoid or a toxoid-linker.

When the enzyme is of the subgroup 3.1.11.6, the prodrug is a compound having the structure:

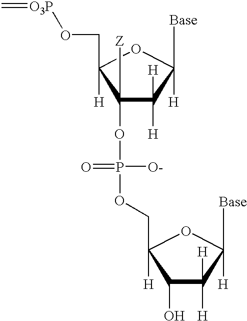

wherein Base is selected from the group consisting of adenine, tyrosine, guanine, cytosine and uracil; and

wherein Z is a toxoid or a toxoid-linker.

When the enzyme is of the subgroup 3.1.13.1, the prodrug is a compound having the structure:

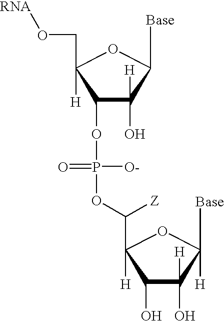

wherein Base is selected from the group consisting of adenine, tyrosine, guanine, cytosine and uracil; and

wherein Z is a toxoid or a toxoid-linker.

When the enzyme is of the subgroup 3.1.21.x, wherein x is selected from the group consisting of 2, 3, 4, and 5, then the prodrug is a compound having the structure:

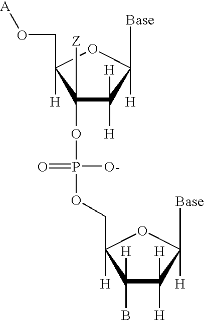

wherein Base is selected from the group consisting of adenine, tyrosine, guanine, cytosine and uracil;

wherein A and B are independently the same or different and are selected from the group consisting of a phosphate and a deoxyribonucleic acid small oligonuleotide; and

wherein Z is a toxoid or a toxoid-linker.

When the enzyme is of the subgroup 3.1.26.x, wherein x is selected from the group consisting of 3, 4, and 5, then the prodrug is a compound having the structure:

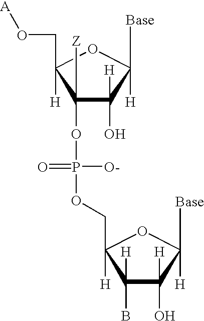

wherein Base is selected from the group consisting of adenine, tyrosine, guanine, cytosine and uracil;

wherein A and B are independently the same or different and are selected from the group consisting of a phosphate and a deoxyribonucleic acid small oligonuleotide; and

wherein Z is a toxoid or a toxoid-linker.

When the enzyme is selected from the subgroup 3.1.26.X, wherein X is selected from the group consisting of 3, 5, and 6, the prodrug is a compound having the structure:

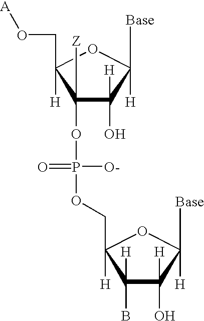

wherein the base is selected from the group consisting of adenine, tyrosine, guanine, cytosine and uracil;

wherein A and B are independently the same or different and is a phosphate or a ribonucleic acid small oligonuleotide; and

wherein Z is a toxoid or toxoid-linker.

When the enzyme is of the subgroup 3.1.27.6, the prodrug is a compound having the structure:

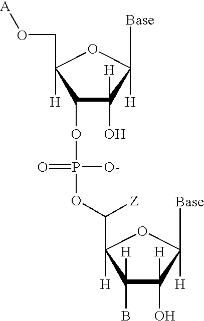

wherein Base is selected from the group consisting of adenine, tyrosine, guanine, cytosine and uracil;

wherein A and B are independently the same or different and are selected from the group consisting of a phosphate and a ribonucleic acid oligonuleotide; and

wherein Z is a toxoid or a toxoid-linker.

When the enzyme is of the subgroup 3.1.31.1, the prodrug is a compound having the structure:

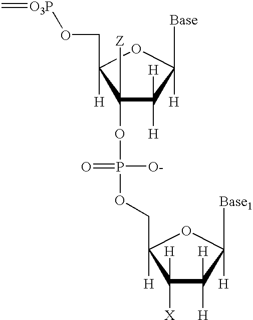

wherein Base is selected from the group consisting of adenine, tyrosine, guanine, cytosine and uracil;

wherein Basel is a toxoid;

wherein X is OH or a phosphate;

wherein Y is H or OH; and

wherein Z is a toxoid or a toxoid-linker.

When the enzyme is of the subgroup 3.2.1.3, the prodrug is a compound having the structure:

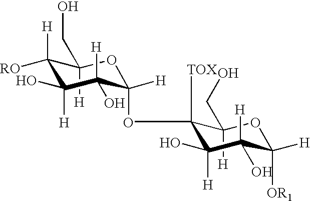

wherein R is H and R1 is a glucose polymer of the formula (glucose)n, wherein n is an integer from 1 to ______; and

wherein TOX is a toxoid.

When the enzyme is of the subgroup 3.2.1.4, the prodrug is a compound having the structure:

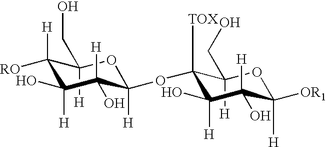

wherein R and R1 are the same or different and is repeating beta-(1,4)-glucose in cellulose; and

wherein TOX is a toxoid.

When the enzyme is of the subgroup 3.2.1.73, the prodrug is a compound having the structure:

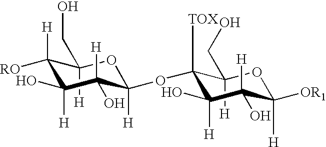

wherein R and R1 are the same or different and are repeating beta-D-glucans containing 1-3 or 1-4 linkages; and

wherein TOX is a toxoid.

When the enzyme is of the subgroup 3.2.1.8, the prodrug is a compound having the structure:

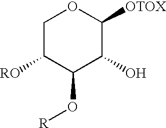

wherein R is H or an oligosaccharide; and

wherein TOX is a toxoid.

When the enzyme is of the subgroup 3.2.1.15, the prodrug is a compound having the structure:

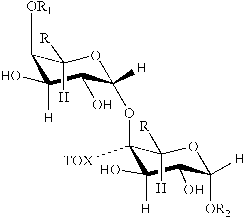

wherein R is CO₂H;

wherein R1 and R2 is polygalacturonic acids linked alpha 1-4; and

wherein TOX is a toxoid.

When the enzyme is of the subgroup 3.2.1.21, the prodrug is a compound having the structure:

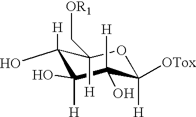

wherein R1 is H or beta-glucosidase; and

wherein TOX is a toxoid.

When the enzyme is of the subgroup 3.2.1.86, the prodrug is a compound having the structure:

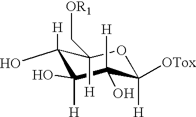

wherein R1 is phosphate or 6-phospho-beta-glucosidase; and

wherein TOX is a toxoid.

When the enzyme is of the subgroup 3.2.1.91, the prodrug is a compound having the structure:

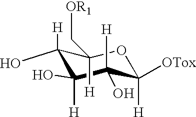

wherein R1 is unspecified; and

wherein TOX is a toxoid.

When the enzyme is of the subgroup 3.2.1.26, the prodrug is a compound having the structure:

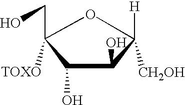

wherein TOX is a toxoid or a glucose derivative.

When the enzyme is of the subgroup 3.2.1.33, the prodrug is a compound having the structure:

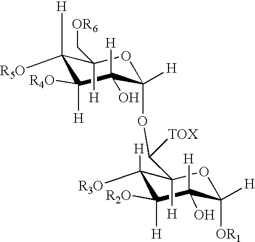

wherein each of R1, R2, R3, R4, R5 and R6 are independently the same or different and is selected from the group consisting of H or a saccharide; and

wherein TOX is a toxoid.

When the enzyme is of the subgroup 3.2.1.55, the prodrug is a compound having the structure:

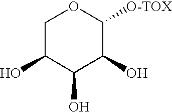

wherein TOX is a toxoid.

When the enzyme is of the subgroup 3.2.1.58, the prodrug is a compound having the structure:

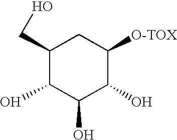

wherein TOX is a toxoid.

When the enzyme is of the subgroup 3.2.1.65, the prodrug is a compound having the structure:

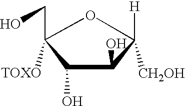

wherein TOX is a toxoid.

When the enzyme is of the subgroup 3.2.1.78, the prodrug is a compound having the structure:

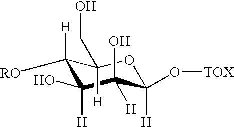

wherein R is unspecified; and

wherein TOX is a toxoid.

When the enzyme is of the subgroup 3.2.1.85, the prodrug is a compound having the structure:

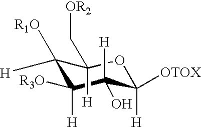

wherein R1 is H;

wherein R2 is PO₃;

wherein R₃ is H; and

wherein TOX is a toxoid.

When the enzyme is of the subgroup 3.2.1.81, the prodrug is a compound having the structure:

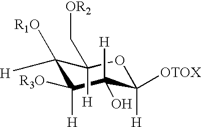

wherein R1 is agarose;

wherein R2 and R3 are each H; and

wherein TOX is a toxoid.

When the enzyme is of the subgroup 3.2.1.83, the prodrug is a compound having the structure:

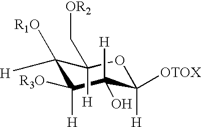

wherein R3 is a carrageen polymer;

wherein R1 is SO₃⁻;

wherein R2 is OH; and

wherein TOX is a toxoid.

When the enzyme is of the subgroup 3.2.1.89, the prodrug is a compound having the structure:

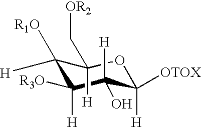

wherein R1 is an arabinogalactan polymer;

wherein R2 and R3 are independently the same or different and is a H or an arabinogalactan polymer; and

wherein TOX is a toxoid.

When the enzyme is of the subgroup 3.2.1.93, the prodrug is a compound having the structure:

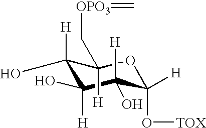

wherein TOX is a toxoid that may alternatively have a saccharide structure.

When the enzyme is of the subgroup 3.2.1.122, the prodrug is a compound having the structure:

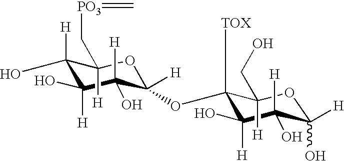

wherein TOX is a toxoid.

When the enzyme is of the subgroup 3.2.1.135, the prodrug is a compound having the structure:

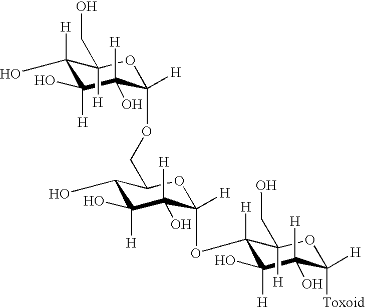

wherein TOX is a toxoid.

When the enzyme is of the subgroup 3.2.2.1, the prodrug is a compound having the structure:

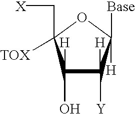

wherein Base is a purine;

wherein X and Y are independently the same or different and is OH or a purine nucleosidase; and

wherein TOX is a toxoid.

When the enzyme is of the subgroup 3.2.2.16, the prodrug is a compound having the structure:

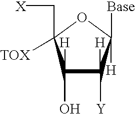

wherein Base is adenine;

wherein X is CH₃S;

wherein Y is OH or a methylthioadenosine nucleosidase; and

wherein TOX is a toxoid.

When the enzyme is of the subgroup 3.2.2.20, the prodrug is a compound having the structure:

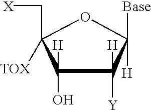

wherein Base is a 3-methylated adenine;

wherein X is deoxyribonucleic acid;

wherein Y is H or DNA-3-methyladenine glycosidase I; and

wherein TOX is a toxoid.

When the enzyme is of the subgroup 3.2.2.23, the prodrug is a compound having the structure:

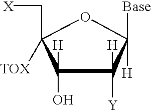

wherein Base is ring-opened N7-methylguanine;

wherein X is deoxyribonucleic acid;

wherein Y is H or formamidopyrimidine-DNA glycosidase; and

wherein TOX is a toxoid.

When the enzyme is of the subgroup 3.2.2.4, the prodrug is a compound having the structure:

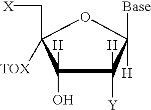

wherein Base is adenine;

wherein X is OPO³;

wherein Y is OH or AMP nucleosidase; and

wherein TOX is a toxoid.

When the enzyme is of the subgroup 3.2.2.9, the prodrug is a compound having the structure:

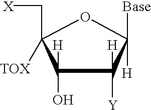

wherein Base is adenine;

wherein X is S-homocysteine;

wherein Y is OH or S-adenosylhomocysteine nucleosidase; and

wherein TOX is a toxoid.

When the enzyme is of the subgroup 3.3.2.1, the prodrug is a compound having the structure:

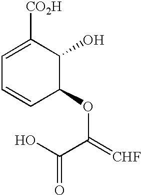

When the enzyme is of the subgroup 3.4.11.10, the prodrug is a compound having the structure:

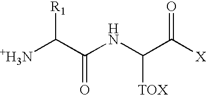

wherein R1 is a leucyl side chain;

wherein R2 is any amino acid;

wherein X is an oligopeptide or a leucyl aminopeptidase; and

wherein TOX is a toxoid.

When the enzyme is of the subgroup 3.4.11.5, the prodrug is a compound having the structure:

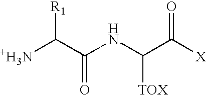

wherein R1 is a proline side chain;

wherein R₂ is any amino acid;

wherein X is an oligopeptide or a proline iminopeptidase; and

wherein TOX is a toxoid.

When the enzyme is of the subgroup 3.4.13.3, the prodrug is a compound having the structure:

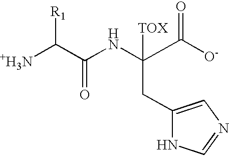

wherein R1 is unspecified; and

wherein TOX is a toxoid.

When the enzyme is of the subgroup 3.4.15.5, the prodrug is a compound having the structure:

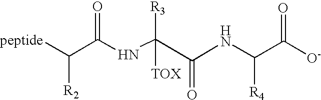

wherein R2, R3 and R4 are unspecified; and

wherein TOX is a toxoid.

When the enzyme is of the subgroup 3.4.16.4, the prodrug is a compound having the structure:

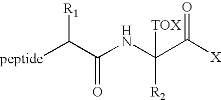

wherein R1 and R2 is a D-Ala amino acid side chain; and

wherein TOX is a toxoid.

When the enzyme is of the subgroup 3.4.17.11, the prodrug is a compound having the structure:

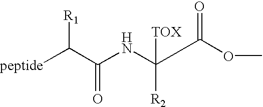

wherein R1 is unspecified;

wherein R2 is a glutamate side chain or glutamate carboxypeptidase; and

wherein TOX is a toxoid.

When the enzyme is of the subgroup 3.4.17.19, the prodrug is a compound having the structure:

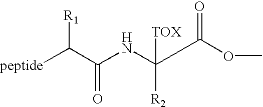

wherein R1 and R2 are unspecified; and

wherein TOX is a toxoid.

When the enzyme is of the subgroup 3.4.19.3, the prodrug is a compound having the structure:

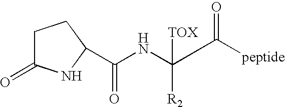

wherein R2 is unspecified; and

wherein TOX is a toxoid.

When the enzyme is of the subgroup 3.4.21.50, the prodrug is a compound having the structure:

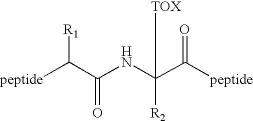

wherein R1 is a lysyl side chain;

wherein R2 is unspecified, and

wherein TOX is a toxoid.

When the enzyme is of the subgroup 3.4.21.53, the prodrug is a compound having the structure:

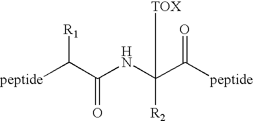

wherein R1 is unspecified or endopeptidase LA

wherein R2 is unspecified; and

wherein TOX is a toxoid.

When the enzyme is of the subgroup 3.4.21.72, the prodrug is a compound having the structure:

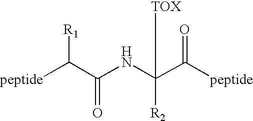

wherein R1 is pro in immunoglobulin A or IgA-specific serine endopeptidase;

wherein R2 is unspecified; and

wherein TOX is a toxoid.

When the enzyme is of the subgroup 3.4.21.88, the prodrug is a compound having the structure:

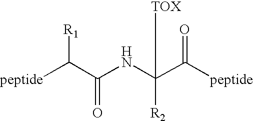

wherein R1 is Ala84 in reprecursor lex A or repressor lexa peptidase;

wherein R2 is unspecified; and

wherein TOX is a toxoid.

When the enzyme is of the subgroup 3.4.21.89, the prodrug is a compound having the structure:

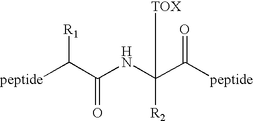

wherein R1 is an N-terminal leader sequence in a signal peptide or signal peptidase I;

wherein R2 is unspecified; and

wherein TOX is a toxoid.

When the enzyme is of the subgroup 3.4.23.23, and the prodrug is a compound having the structure:

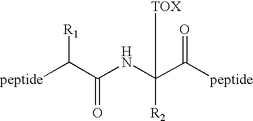

wherein R1 and R2 are independently the same or different and is a hydrophobic side chain or mucorpepsin; and

wherein TOX is a toxoid.

When the enzyme is of the subgroup 3.4.23.36, the prodrug is a compound having the structure:

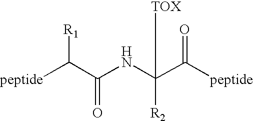

wherein R1 is unspecified;

wherein R2 is a cysteinyl side chain or signal peptidase II; and

wherein TOX is a toxoid.

When the enzyme is of the subgroup 3.4.24.X, wherein X is selected from the group consisting of 25, 26, 28, and 36, the prodrug is a compound having the structure:

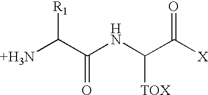

wherein R1 is unspecified; and

wherein TOX is a toxoid.

When the enzyme is of the subgroup 3.4.24.55, the prodrug is a compound having the structure:

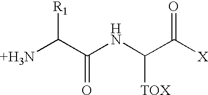

wherein R1 is tyrosine or phenylalanine; and

wherein TOX is a toxoid.

When the enzyme is of the subgroup 3.4.24.57, the prodrug is a compound having the structure:

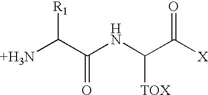

wherein R1 is arginine; and

wherein TOX is a toxoid.

When the enzyme is of the subgroup 3.4.24.70, the prodrug is a compound having the structure:

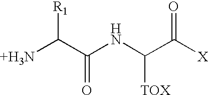

wherein R1 is glycine or alanine; and

wherein TOX is a toxoid.

When the enzyme is of the subgroup 3.5.1.X, wherein X is selected from the group consisting of 1, 10, 11, 16, 18, 19, 24, 25, 31, 32, 38, 14, 46, 15, 54, 6, 68, 78, and 81, the prodrug is a compound having the structure:

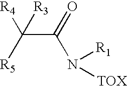

wherein R1, R3, R4 and R5 are unspecified; and

wherein TOX is a toxoid.

When the enzyme is of the subgroup 3.5.2.X, wherein X is selected from the group consisting of 5, 6, 7, and 10, the prodrug is a compound having the structure:

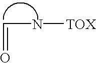

wherein TOX is a toxoid.

When the enzyme is of the subgroup 3.5.3.4, the prodrug is a compound having the structure:

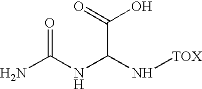

wherein TOX is a toxoid.

When the enzyme is of the subgroup 3.5.3.6, the prodrug is a compound having the structure:

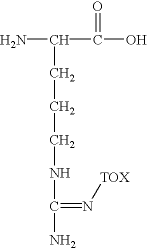

wherein TOX is a toxoid.

When the enzyme is of the subgroup 3.5.3.8, the prodrug is a compound having the structure:

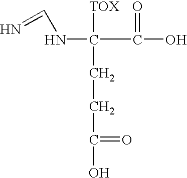

wherein TOX is a toxoid.

When the enzyme is of the subgroup 3.5.3.9, the prodrug is a compound having the structure:

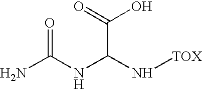

wherein TOX is a toxoid.

When the enzyme is of the subgroup 3.5.3.11, the prodrug is a compound having the structure:

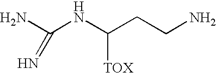

wherein TOX is a toxoid.

When the enzyme is of the subgroup 3.11.1.1, the prodrug is a compound having the structure:

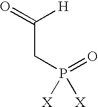

wherein X is NHCH2CH2Cl.

When the enzyme is of the subgroup 4.1.1.1, the prodrug is a compound having the structure:

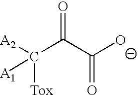

wherein A1 and A2 are independently the same or different and is unspecified; and

wherein TOX is a toxoid.

When the enzyme is of the subgroup 4.1.1.18, the prodrug is a compound having the structure:

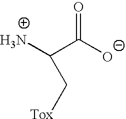

wherein TOX is a toxoid.

When the enzyme is of the subgroup 4.1.2.19, the prodrug is a compound having the structure:

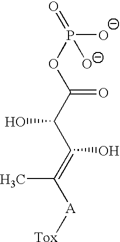

wherein TOX is a toxoid.

When the enzyme is of the subgroup 4.1.2.25, the prodrug is a compound having the structure:

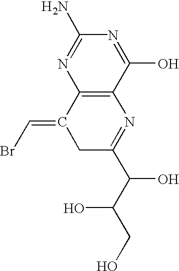

When the enzyme is of the subgroup 4.1.3.30, the prodrug is a compound having the structure:

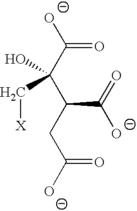

wherein X is selected from the group consisting of Cl, Br, I and F.

When the enzyme is of the subgroup 4.1.3.27, the prodrug is a compound having the structure:

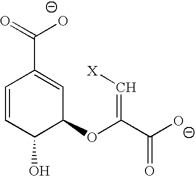

wherein X is selected from the group consisting of Cl, Br, I and F.

When the enzyme is of the subgroup 4.1.3.16, the prodrug is a compound having the structure:

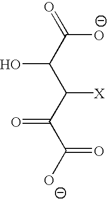

wherein X is selected from the group consisting of Cl, Br, I and F.

When the enzyme is of the subgroup 4.1.99.1, the prodrug is a compound having the structure:

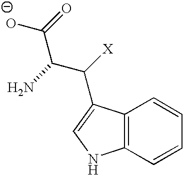

wherein X is selected from the group consisting of Cl, Br, I and F.

When the enzyme is of the subgroup 4.1.99.4, the prodrug is a compound having the structure:

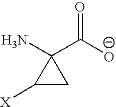

wherein X is selected from the group consisting of Cl, Br, I and F.

When the enzyme is of the subgroup 4.2.1.51, the prodrug is a compound having the structure:

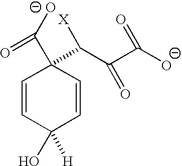

wherein X is selected from the group consisting of Cl, Br, I and F.

When the enzyme is of the subgroup 4.2.99.11, the prodrug is a compound having the structure:

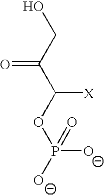

wherein X is selected from the group consisting of Cl, Br, F or I.

When the enzyme is of the subgroup 4.2.99.2, the prodrug is a compound having the structure:

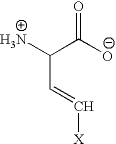

wherein X is selected from the group consisting of Cl, Br, F or I.

When the enzyme is of the subgroup 4.4.1.8, the prodrug is a compound having the structure:

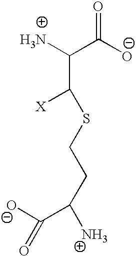

wherein X is selected from the group consisting of Cl, Br, F or I.

When the enzyme is of the subgroup 4.4.1.11, the prodrug is a compound having the structure:

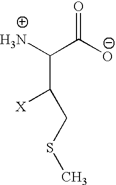

wherein X is selected from the group consisting of Cl, Br, F, and I.

While the invention has been described in detail and with reference to specific embodiments thereof, it will be apparent to one skilled in the art that various changes and modifications can be made therein without departing from the spirit and scope thereof.

References

Amyes, S. G. et al. (1992) J Med Microbiol. 36(1):1-3

Armstrong et al. (1985) J. Chem. Soc., Perkin Trans. 1:691

Armstrong, D. et al. (1999) INFECTIOUS DISEASES (edited by Mosby)

Arthur M., et al. (1999) Antimicrob Agents Chemother. 43(8):1875-80

Aulabaugh, A. et al. (1990) Biochemistry 29(11):2824-30

Banerjee, D. et al. (1995) Acta Biochem Pol. 42(4):457-64

Behrens, C. H. et al. J. Org. Chem. (1985) 50:5696

Berchtold, G. A. et al. (1982) J. Am. Chem. Soc. 104:1153-1154; 7036-7041

Berger-Bachi, B. et al. (1989) Mol. Gen. Genet. 219(1-2):263-9

Bertino, J. R. et al. (1996) Stem Cells 14(1):5-9

Blight, K. J. et al. (1998) Antivir. Ther. 3(Suppl 3):71-81

Bloomer, J. L. et al. (1976) J. Chem. Soc., Perkin Trans. 1, 14:1485-1491

Bohacek, R. S. et al. (1997) Curr. Opin. Chem. Biol. 1(2):157-61

Bonomo, R. A. et al. (1999) Front Biosci. 4:34-41

Carlson, et al. J. Org. Chem. (1981) 46:3936

Casado et al. (2000) AIDS. 14(2):F 1-7

Casadewell, B et al. (1999) J Bacteri. 181(12):3644-8

Caceres, N. E. et al. (1997) J. Bacteriol. 179(16):5046-55

Chang, A. K. et al. (1998) Biochem. J. 333 (Pt 3):765-77

Chipman, D. et al. (1998) Biochem. Biophys. Acta. 1385(2):401-19

Coen, D. M. Nucleosides and Foscarnet-Mechanisms in ANTIVIRAL DRUG RESISTANCE

Cohen, N. et al. (1983) J. Am. Chem. Soc. 105:3661-3672

Das, B. et al. (2000) Cancer Invest. 18(2):115-22

DeGraw, J. I. et al. (1986) J. Heterocycl. Chem 23:1-4

Taylor, E. C. et al. (1985) J. Med. Chem., 28:914-921

Dötz, K. H. et al. (1999) Organomet. Chem. 578:223-228

Ekins, S. et al. (1999) Pharmacogenetics 9(4):477-89

Ekins, S. et al. (1999) J. Pharmacol. Exp. Ther. 291(1):424-33

Evans, M. E. et al. (1967) Carbohydr. Res. 3:453

Freer, A. A. et al. (1996) J. Chem. Soc., Perkin Trans. 1, 17:2111-2116

Ganem, B. et al. (1982) J. Am. Chem. Soc. 104:6787-6788

Gypser, A. et al. (1994) Liebigs. Ann. Chem., 775-780

Gysper, A. et al. (1997) J. Chem. Soc., Perkin Trans. 1., 1013-1016

Halgand, F. et al. (1999) Biochemistry 38(19):6025-34

Hanaki, H. et al. (1998) Antimicrob Chemother. 42(2):199-209

Hanessian, S. et al. (1983) Can. J. Chem. 61:634

Harms, C. T. et al. (1992) Mol. Gen. Genet. 1(3):427-35

Heath, R. J. et al. (2000) J. Biol. Chem. 275(7):4654-9

Hill, C. M. et al. (1997) Biochem J. 327 (Pt 3):891-8

Ibdah, M. et al. (1996) Biochemistry 35(50):16282-91

Jackrnan A. L. et al. (1997) Clin. Cancer. Res. 3(6):911-21

Josefsson, E. et al. (1999) J. Periodontal Res. 34(7):387-92

Kirkpatrick, D. L. et al. (1999) Comb. Chem. High Throughput Screen 2(4):211-21

Kobayashi, H. et al. (1995) Jpn. J. Cancer Res. 86(11):1014-8

Kleanthous, C. et al. (1985) Biochemistry 24(20):5307-13

Lessard I. A. et al. (1999) Biochemistry 38(42):14006-22

Lewbart, M. L. et al. (1969) J. Org. Chem. 34:3505

Lipshutz, B. H. et al. (1988) J. Org. Chem. 53:4495.

Lonn, U. et al. (1996) Cancer 77(1):107-12

Louie, A. et al. (1999) Antimicrob. Agents Chemother. 43(12):2841-7

Malik, A. H. et al. (2000) Ann. Intern. Med. 132(9):723-31

Markowitz, M. et al. (1996) Antiviral Drug Resistance, Richman D. D. ed. John Wiley and Sons, LTD

McKague, A. B. (1999) Synth. Commun., 29(9):1463-1475

McVie J. G. (1999) Cancer Treat. Rev. 25(6):323-31

Mdluli, K. et al. (1998) Mol. Microbiol. 27(6):1223-33

Murray, B. (1997) Adv. Intern. Med. 42: 339-67

Miesel, L. et al. (1998) Novartis Found. Symp. 217:209-20;discussion 220-1

Montravers, P. et al. (1999) Infect. Immun. 67(4):1579-84

Mulligan, K. et al. (2000) J. Acquir. Immune Defic. Syndr. 23(l):35-43

Nelson, D. L. et al. Principles of Biochemistry. 2000, ed. Lehninger,

Neu, H. C. (1992) Science 257:1064-1073

Oram, R. J. et al. (2000) J. Infect. Dis. 181(4):1458-61

Pang, S. S. et al. (1999) Biochemistry 38(16):5222-31

Papamichael, D. (1999) Oncologist 4(6):478-87

Patrick, T. B. et al. (1994) J. Org. Chem. 59(5):1210-1212

Pirrung et al. (1991) J. Am. Chem. Soc. 111: 1020

Poulsen, C. et al. (1989) Eur. J. Biochem. 185(2):433-9

Ruas, M. et al. (1998) Biochem. Biophys. Acta. 378(2):F1 15-77

Raynaud, C. et al. (1999) Microbiology 145(Pt 6):1359-67

Sambrook et al., “Molecular Cloning: A Laboratory Manual”, 2^{nd} Edition.

Sausville, E. A. et al. (1999) Pharmacol Ther. 82(2-3):285-92

Schaechter, M. (1993) Mechanisms ofMicrobial Disease ed. Williams & Wilkins

Schmit, I. et al. (1999) J. Infect. Dis. 180(2):487-90

Schmitz, F. J. et al. (1999) In Infectious Diseases, ed. D. Armstrong and J. Cohen

Shaner, D. L. et al. Biochemistry and Molecular Biology Roe, R. M. et al., (Eds.) IOS Press, 1997

Shaw, K. J. et al. (1980) J. Bacteriol. 141(3):1258-63

Shaw, W. V. et al. (1988) Biochem Soc Trans. 16(6):939-42

Shaw, W. V. et al. (1991) Annu. Rev. Biophys. Biophys. Chem. 20:363-86

Shirasaka, T. et al. (1995) Proc Natl Acad Sci U S A. 92(6):2398-402

Simon, S. M. et al. (1994) Proc. Natl. Acad. Sci. U S A. 91(9):3497-504

Smith, K. A. et al. (1995) Philos. Trans. R. Soc. Lond B. Biol. Sci. 347(1319):49-56

Spector D. L. et al. (1998) Culture and Biochemical Analysis of Cells. A Laboratory Manual, Cold Spring Harbor Laboratory Press

Staschke et al. (1995) Virology 214(2):642-6

Stühlinger, M. et al. (1994) J. Steroid Biochem. Mol. Biol. 49(1):39-42

Sugarman, B. J. et al. (1985) Science 230:943-945

Svendsen, A. et al. (1975) J. Org. Chem. 40(13):1927-1932

Takabatake, T. et al. (June 1992) Chem. Pharm. Bull. 40(6):1644-6

Tamburrini, E, et al. (1999) Med. Microbiol Immunol. (Berl). 188(1):1-7 Tetrahedron (1967) 23:359

Totsuka, K. et al. J. (1999) Antimicrob. Chemother. 44(4):455-60

VanRheenen, V. et al. (1976) Tetrahedron Lett. 23:1973-1976

Varghese, J. N. et al. (1998) Structure 6(6):735-46

Venturi, G. et al. (2000) J. Infect. Dis. 181(2):740-5

Voet, D. et al. (1995) Biochemistry John Wiley & Sons, Inc.

Vollmer, M. D. et al. (1994) J. Bacteriol. 176(14):4366-4375

Vollmer, M. D. et al. (1995) J. Bacteriol. 177(10):2938-2941

Vollmer, M. D. et al. (1998) Appl. Environ. Microbiol. 64(9), 3290-3299

Wakselman, C. et al. (1982) Journal of Fluorine Chemistry 21(82):99-106

Weinstock, O. et al. (1992) J Bacteriol. 174(17):5560-6

Wettergren, Y. et al. (1994) Somat Cell Mol Genet. 20(4):267-85

Whitcomb, C. E. (1999) Toxicol Ind Health. 15(1-2):231-9

Yuan, Y. et al. (1995) Proc Natl Acad Sci USA 92(14):6630-4

Yen, Y. et al. (1994) Cancer Res. 54(14):3686-91

Zhdanov, Y. A. et al. (1971) Zh. Obshch. Khim 41(8):1844-1848

Zimbelman, J. et al. (1999) Pediatr. Infect. Dis. J. 18(12):1096-100

1. A method for inhibiting the proliferation of a pathogen or a cell infected with an pathogen, wherein the pathogen expresses an iECTA enzyme, comprising contacting the pathogen or the cell with an effective amount of an iECTA prodrug that is activated to a toxin by the pathogen or in the cell by the iECTA enzyme, thereby inhibiting the proliferation of the pathogen or the cell.

2. The method of claim 1, wherein the iECTA enzyme is selected from the group of enzymes listed in FIGS. 7A and B and their biological equivalents.

3. The method of claim 2, wherein the iECTA enzymes beta-lactamase and peptide deformylase are specifically excluded.

4. The method of claim 1, wherein the iECTA enzyme is a member selected from the group consisting of the enzymes designated EC1, EC2, EC3, EC4, EC5, and EC6.

5. The method of claim 1, wherein the pathogen is selected from the group consisting of bacteria, parasites, rickettesia, virus, and fungus.

6. The method of claim 1, wherein the contacting is in vitro or in vivo.

7. A method for screening for a therapeutic agent that selectively inhibits the growth of pathogen or a pathogen-infected cell, comprising contacting the pathogen or the cell with an effective amount of an iECTA prodrug that is activated to a toxin by the pathogen or in the cell by the iECTA enzyme and assaying for inhibition growth of the pathogen or the cell.

8. The method of claim 7, wherein the iECTA enzyme is selected from the group of enzymes listed in FIGS. 7A and 7B, and their biological equivalents.

9. The method of claim 7, wherein the enzyme is a member of an enzyme selected from the group consisting of the enzymes designated EC1, EC2, EC3, EC4, EC5, and EC6.

10. The method of claim 7, wherein the iECTA enzymes beta-lactamase and peptide deformylase are specifically excluded.

11. The method of claim 7, wherein the enzyme is a member of the group consisting of the enzymes designated EC1, EC2, EC3, EC4, EC5, and EC6.

12. The method of claim 7, wherein the pathogen is selected from the group consisting of bacteria, parasites, rickettesia, virus, and fungus.

13. The method of claim 7, wherein the contacting is in vitro or in vivo.

14. The method of claim 7, further comprising delivering to a normal, non-infected counterpart cell to the infected host cell, an effective amount of the iECTA prodrug and assaying the normal, non-infected cell for inhibition of cell growth or cytotoxicity.

15. The method of claim 7, wherein the host cell is infected with a pathogen that expresses or induces the expression of an enzyme that is selectively expressed by the pathogen.

16. The method of claim 14, wherein the normal, non-infected counterpart is a plant cell or an animal cell.

17. The method of claim 16, wherein the animal cell is a mammalian cell.

18. A method for identifying drug targets, comprising:
a. searching a first data structure to obtain a first set of information, wherein the first set of information comprises first enzymes associated with a target organism or in a pathogen-infected cell;
b. searching one or more data structures to obtain one or more sets of information, wherein the one or more sets of information comprises one or more expressed enzymes associated with one or more respective classes of organisms that is different that the target organism; and
c. comparing the first set of information to the one or more sets of information to create a first output, wherein the first output comprises target enzymes in the first set of information that are not present in the one or more sets of information, and wherein the target enzymes are drug targets.

19. The method of claim 18, wherein the enzymes are expressed by the target organism or in a pathogen infected cell, but absent in the classes of organism of step b.

20. The method of claim 18, wherein the target organism is selected from the group consisting of bacteria, parasites, rickettesia, virus, and fungus.

21. The method of claim 18, wherein the organism of step b is an animal or plant.

22. The method of claim 18, wherein the animal is a mammal.

23. The method of claim 18, wherein the comparison step utilizes an alignment search algorithm.

24. The method of claim 18, wherein the alignment search algorithm is a Needleman-Wunsch global alignment algorithm, a Smith-Waterman local alignment algorithm, a “FAST” algorithm, or a BLAST algorithm.

25. The method of claim 18, further comprising the step of outputting a list of the target enzymes.

26. The method of claim 18, further comprising the step of comparing the first output to a data structure of metabolic enzymes, wherein the metabolic data structure contains enzymes present in metabolic pathways, to obtain a set of metabolic target enzymes, wherein the metabolic target enzymes are enzymes present in both the first output and in the metabolic data structure, and a set of non-metabolic target enzymes, wherein the non-metabolic target enzymes are enzymes present in the first output but not in the metabolic data structure.

27. The method of claim 26, further comprising the step of displaying the metabolic enzymes and the non-metabolic enzymes in a manner such that the metabolic enzymes are distinguishable from the non-metabolic enzymes.

28. The method of claim 18, wherein the searching steps utilize a network.

29. The method of claim 18, wherein the network is capable of searching the one or more data structures, wherein the one or more data structures are stored on a plurality of servers connected to the network.

30. The method of claim 18, wherein the comparing step utilizes a user's computer.

31. The method of claim 18, wherein the first set of information comprises information about Enzyme Commission numbers relating to the first set of enzymes associated with the target organism.

32. The method of claim 18, wherein the one or more data structures comprise information about Enzyme Commission numbers relating to the one or more expressed enzymes.

33. The method of claim 18, wherein the one or more data structures comprises a public domain database.

34. The method of claim 18, further comprising the additional step of using target enzymes to design iECTA compounds.

35. A method for identifying drug targets, the method comprising:
a. searching the first data structure to obtain the first set of information, wherein the first set of information comprises first enzymes associated with a target organism;
b. searching a second data structure to obtain a second set of information, wherein the second set of information comprises second enzymes associated with a first class of organism;
c. comparing the first set of information to the second set of information to create the first output, wherein the first output comprises enzymes in the first set of information that are not present in the second set of information, and wherein the identified enzymes are drug targets;
d. searching a third data structure to obtain a third set of information relating to third expressed enzymes associated with a second class of organism, wherein the second class of organism is different from the first class of organism, wherein the third expressed enzymes are expressed at elevated levels in the second class of organism; and
e. comparing the first output with the third set of information to create a second output, wherein the second output identifies enzymes in the first output that are not present in the third set of information, and wherein the target enzymes are drug targets.

36. The method of claim 35, further comprising repeating steps (d)-(e) n times, wherein there are n data structures.

37. The method of claim 35, wherein the drug targets are Enzyme Catalyzed Therapeutic Activation (“ECTA”) targets.

38. The method of claim 35, wherein the comparison step utilizes an alignment search algorithm.

39. The method of claim 38, wherein the alignment search algorithm is a Needleman-Wunsch global alignment algorithm, a Smith-Waterman local alignment algorithm, a “FAST” algorithm, or a BLAST algorithm.

40. The method of claim 35, further comprising the step of outputting a list of the target enzymes.

41. The method of claim 35, further comprising the step of comparing the target enzymes to a data structure of metabolic enzymes, wherein the metabolic data structure contains enzymes present in metabolic pathways, to obtain a set of metabolic target enzymes, wherein the metabolic target enzymes are both target enzymes and present in the metabolic data structure, and a set of non-metabolic target enzymes, wherein the non-metabolic target enzymes are target enzymes but are not in the metabolic data structure.

42. The method of claim 41, further comprising the step of displaying the metabolic enzymes and the non-metabolic enzymes in a manner such that the metabolic enzymes are distinguishable from the non-metabolic enzymes.

43. The method of claim 35, wherein the searching steps utilize a network.

44. The method of claim 35, wherein the network is capable of searching the n data structures, wherein the n data structures are stored on a one or more servers connected to the network.

45. The method of claim 35, wherein the comparing steps utilize a user's computer.

46. The method of claim 35, wherein the first set of information comprises information about Enzyme Commission numbers relating to the first enzymes associated with the target organism.

47. The method of claim 35, wherein any of the n data structures in the group comprises information about Enzyme Commission numbers.

48. The method of claim 35, wherein any of the n data structures comprise a public domain database.

49. The method of claim 35, wherein the class of organism is an animal or a plant.

50. The method of claim 49, wherein the animal is a mammal.

51. The method of claim 35, further comprising the step of using the target enzymes to design ECTA compounds.

52. A system for identifying enzymes for designing Enzyme Catalyzed Therapeutic Activation (ECTA) compounds, comprising:
a. searching a first data structure to obtain a first set of information, wherein the first set of information comprises first enzymes associated with a target organism;
b. searching one or more data structures to obtain one or more sets of information, wherein the one or more sets of information comprises one or logic for searching a first data structure to obtain a first set of information relating to one or more enzymes associated with a target organism;
c. logic for searching one or more other data structures to obtain one or more additional sets of information relating to one or more expressed enzymes associated with one or more additional classes of organisms; and
d. logic for comparing the first set of information to the one or more additional sets of information to identify enzymes in the first set of information that are not present in the one or more additional sets of information, wherein the identified enzymes are capable of being used to design ECTA compounds.

53. The system of claim 52, further comprising logic for outputting a list of the identified enzymes.

54. The system of claim 52, further comprising: logic for organizing the identified enzymes into a first set of enzymes capable of being placed into metabolic pathways and a second set of enzymes not capable of being placed into metabolic pathways; and logic for displaying the first and second sets of enzymes such that the first set of enzymes are distinguishable from the second set of enzymes.

55. The system of claim 52, wherein a third data structure is queried to organize the identified enzymes.

56. The system of claim 52, wherein a network is utilized to search at least one of the first data structure and the second data structure.

57. The system of claim 56, wherein the network is capable of communicating utilizing TCP/IP or IPX protocols.

58. The system of claim 52, wherein the information relating to the one or more enzymes of the target organism includes information about Enzyme Commission (EC) numbers of the one or more enzymes.

59. The system of claim 52, wherein the one or more additional sets of information relating to the one or more expressed enzymes associated with one or more classes of organisms includes information about Enzyme Commission (EC) numbers of the one or more expressed enzymes.

60. A computer program product for identifying enzymes for designing Enzyme Catalyzed Therapeutic Activation (ECTA) compounds, comprising:
a. computer code for searching a first data structure to obtain a first set of information relating to one or more enzymes associated with a target organism;
b. computer code for searching one or more other data structures to obtain one or more additional sets of information relating to one or more expressed enzymes associated with one or more additional classes of organisms; and
c. computer code for comparing the first set of information to the one or more additional sets of information to identify enzymes in the first set of information that are not present in the one or more additional sets of information, wherein the identified enzymes are capable of being used to design ECTA compounds.

61. The computer program product of claim 60, further comprising computer code for outputting a list of the identified enzymes.

62. The computer program product of claim 60, further comprising:
computer code for organizing the identified enzymes into a first set of enzymes capable of being placed into metabolic pathways and a second set of enzymes not capable of being placed into metabolic pathways; and computer code for displaying the first and second sets of enzymes such that the first set of enzymes are distinguishable from the second set of enzymes.

63. The computer program product of claim 60, wherein a third data structure is queried to organize the identified enzymes.

64. The computer program product of claim 60, wherein a network is utilized to search at least one of the first data structure and the second data structure.

65. The computer program product of claim 64, wherein the network is capable of communicating utilizing TCP/IP or IPX protocols.

66. The computer program product of claim 60, wherein the information relating to the one or more enzymes of the target organism includes information about Enzyme Commission (EC) numbers of the one or more enzymes.

67. The computer program product of claim 60, wherein the one or more additional sets of information relating to the one or more expressed enzymes associated with one or more classes of organisms includes information about Enzyme Commission (EC) numbers of the one or more expressed enzymes.

68. A method for alleviating the symptoms of a disease related to an organism or host cell expressing an iECTA enzyme in a subject comprising administering to the subject an effective amount of an iECTA prodrug that is activated to a toxin in the organism or cell by the iECTA enzyme, thereby alleviating the symptoms.

69. The method of claim 68, wherein the iECTA enzyme is selected from the group of enzymes listed in FIGS. 7A and B and their biological equivalents.

70. The method of claim 69, wherein the iECTA enzymes beta-lactamase and peptide deformylase are specifically excluded.

71. The method of claim 69, wherein the iECTA enzyme is a member of an enzyme selected from the group consisting of the enzymes designated EC1, EC2, EC3, EC4, EC5, and EC6.

72. The method of claim 69, wherein the organism is selected from the group consisting of bacteria, parasites, rickettesia, virus, and fungus.

73. The method of claim 69, wherein the host is an animal or plant.

74. The method of claim 73, wherein the animal is a mammal.

75. A method for treating an infection caused by a pathogen expressing an iECTA enzyme or a host cell expressing an iECTA enzyme in a subject, comprising administering to the subject an effective amount of an iECTA prodrug that is activated to a toxin in the pathogen or cell by the iECTA enzyme, thereby alleviating the symptoms.

76. The method of claim 75, wherein the iECTA enzyme is selected from the group of enzymes listed in FIGS. 7A and B and their biological equivalents.

77. The method of claim 75, wherein the iECTA enzymes beta-lactamase and peptide deformylase are specifically excluded.

78. The method of claim 75, wherein the iECTA enzyme is a member of an enzyme selected from the group consisting of the enzymes designated EC1, EC2, EC3, EC4, EC5, and EC6.

79. The method of claim 75, wherein the organism is selected from the group consisting of bacteria, parasites, rickettesia, virus, and fungus.

80. The method of claim 75, wherein the host is an animal or plant.

81. The method of claim 80, wherein the animal is a mammal.
